(19) United States
(12) Patent Application Publication
(10) Pub. No.: US 2024/0382513 A1
(43) Pub. Date: Nov. 21, 2024

(54) MUSCLE TARGETING COMPLEXES AND USES THEREOF FOR MODULATION OF GENES ASSOCIATED WITH MUSCLE HEALTH

(71) Applicant: Dyne Therapeutics, Inc., Waltham, MA (US)

(72) Inventors: Romesh R. Subramanian, Framingham, MA (US); Mohammed T. Qatanani, Waltham, MA (US); Cody A. Desjardins, Waltham, MA (US); Duncan Brown, Berkeley, CA (US); Victor Kotelianski, Newton, MA (US); Timothy Weeden, Waltham, MA (US); Brendan Quinn, Boston, MA (US); John Najim, Waltham, MA (US)

(73) Assignee: Dyne Therapeutics, Inc., Waltham, MA (US)

(21) Appl. No.: 18/577,382

(22) PCT Filed: Jul. 1, 2022
(86) PCT No.: PCT/US2022/073362

§ 371 Date: Jan. 8, 2024

Related U.S. Application Data
(60) Provisional application No. 63/220,039, filed on Jul. 9, 2021.
(60) Provisional application No. 63/220,050, filed on Jul. 9, 2021.
(60) Provisional application No. 63/220,056, filed on Jul. 9, 2021.
(60) Provisional application No. 63/220,071, filed on Jul. 9, 2021.
(60) Provisional application No. 63/220,085, filed on Jul. 9, 2021.

(51) Int. Cl. A61K 31/713; A61K 47/68; C07K 16/28; C12N 15/113

(52) U.S. Cl. CPC A61K 31/713; A61K 47/6807; A61K 47/6849; C07K 16/2881; C12N 15/113; C07K 2317/55; C07K 2317/56; C07K 2317/92; C12N 2310/11; C12N 2310/14; C12N 2310/315; C12N 2310/33

(57) ABSTRACT

Aspects of the disclosure relate to molecular payloads that modulate the expression or activity of genes involved in muscle growth and maintenance (e.g., MSTN, INHBA, ACVR1B, MLCK1, ACVR1, FBXO32, TRIM63, MEF2D, KLF15, MED1, MED13, and/or PPP1R3A), and complexes comprising a muscle-targeting agent covalently linked to such molecular payloads. In some embodiments, the muscle-targeting agent specifically binds to an internalizing cell surface receptor on a muscle cell (e.g., a cardiac muscle cell, a smooth muscle cell, a skeletal muscle cell). In some embodiments, the molecular payload is an oligonucleotide, such as an antisense oligonucleotide or RNAi oligonucleotide.

### RELATED APPLICATIONS

This application claims priority under 35 U.S.C. § 119(e) to U.S. Provisional Application No. 63/220,050, entitled “MUSCLE TARGETING COMPLEXES AND USES THEREOF FOR MODULATION OF GENES ASSOCIATED WITH MUSCLE HEALTH”, filed on Jul. 9, 2021; U.S. Provisional Application No. 63/220,039, entitled “MUSCLE TARGETING COMPLEXES AND USES THEREOF FOR MODULATION OF MLCK1”, filed on Jul. 9, 2021; U.S. Provisional Application No. 63/220,056, entitled “MUSCLE TARGETING COMPLEXES AND USES THEREOF FOR MODULATION OF ACVR1”, filed on Jul. 9, 2021; U.S. Provisional Application No. 63/220,071, entitled “MUSCLE TARGETING COMPLEXES AND USES THEREOF FOR MODULATION OF GENES ASSOCIATED WITH MUSCLE ATROPHY”, filed on Jul. 9, 2021; and U.S. Provisional Application No. 63/220,085, entitled “MUSCLE TARGETING COMPLEXES AND USES THEREOF FOR MODULATION OF GENES ASSOCIATED WITH CARDIAC MUSCLE DISEASE”, filed on Jul. 9, 2021 the contents of each of which are incorporated herein by reference in their entirety.

### FIELD OF THE INVENTION

The present application relates to molecular payloads (e.g., oligonucleotides) that modulate the expression or activity of genes (e.g., MSTN, INHBA, ACVR1B, MLCK1, ACVR1, FBXO32, TRIM63, MEF2D, KLF15, MED1, MED13, or PPP1R3A) associated with muscle health (e.g., muscle growth and maintenance) and targeting complexes for delivering such molecular payloads (e.g., oligonucleotides) to cells (e.g., cardiac, smooth, and/or skeletal muscle cells) and uses thereof, particularly uses relating to treatment of disease.

### REFERENCE TO AN ELECTRONIC SEQUENCE LISTING

The contents of the electronic sequence listing (D082470057W000-SEQ-ZJG.xml; Size: 2,799,490 bytes; and Date of Creation: Jul. 1, 2022) is herein incorporated by reference in its entirety.

### BACKGROUND

The expression and/or activity of several genes, including myostatin (MSTN), inhibin beta A (INHBA), activin receptor type-1B (ACVR1B), myosin light chain kinase (MLCK1), activin A receptor type-1 (ACVR1), atrogin-1 (FBXO32), tripartite motif containing 63 (TRIM63), myocyte-specific enhancer factor 2D (MEF2D), Kruppel-like factor 15 (KLF15), Mediator complex subunit 1 (MED1), Mediator complex subunit 13 (MED13), and protein phosphatase 1 regulatory subunit 3A (PPP1R3A), have been implicated in various aspects of muscle health. Aberrant expression of one or more of these genes, or expression of a mutated form thereof, may be involved in various muscle disorders, including cardiac and skeletal muscle disorders such as cardiac fibrosis, cardiac muscle atrophy, and skeletal muscle atrophy, among others.

### SUMMARY

According to some aspects, the disclosure provides molecular payloads (e.g., oligonucleotides) that modulate the expression or activity of genes (e.g., MSTN, INHBA, ACVR1B, MLCK1, ACVR1, FBXO32, TRIM63, MEF2D, KLF15, MED1, MED13, or PPP1R3A) associated with muscle health (e.g., muscle growth and maintenance) and complexes that target muscle cells (e.g., cardiac and/or skeletal muscle cells) for the purposes of delivering molecular payloads to those cells. In some embodiments, complexes provided herein are designed to target cardiac muscle cells. In some embodiments, complexes provided herein are designed to target skeletal muscle cells. In some embodiments, complexes provided herein are particularly useful for delivering molecular payloads that modulate (e.g., reduce) the expression (e.g., protein and/or RNA level) or activity of genes involved in muscle health, such as muscle growth and maintenance. Such genes include, but are not limited to: MSTN, INHBA, ACVR1B, MLCK1, ACVR1, FBXO32, TRIM63, MEF2D, KLF15, MED1, MED13, and PPP1R3A. In some embodiments, the disclosure provides complexes that target muscle cells for the purposes of delivering molecular payloads that modulate the expression of one or more MSTN, INHBA, ACVR1B, MLCK1, ACVR1, FBXO32, TRIM63, MEF2D, KLF15, MED1, MED13, and PPP1R3A.

Some aspects of the present disclosure provide complexes comprising an anti-transferrin receptor 1 antibody covalently linked to a molecular payload that modulates the expression or activity of myostatin (MSTN), inhibin beta A (INHBA), activin receptor type-1B (ACVR1B), myosin light chain kinase (MLCK1), activin A receptor type-1 (ACVR1), atrogin-1 (FBXO32), tripartite motif containing 63 (TRIM63), myocyte-specific enhancer factor 2D (MEF2D), Kruppel-like factor 15 (KLF15), Mediator complex subunit 1 (MED1), Mediator complex subunit 13 (MED13), and/or protein phosphatase 1 regulatory subunit 3A (PPP1R3A) wherein the antibody comprises:
(i) a heavy chain variable region (VH) comprising an amino acid sequence at least 95% identical to SEQ ID NO: 76; and/or a light chain variable region (VL) comprising an amino acid sequence at least 95% identical to SEQ ID NO: 75;
(ii) a heavy chain variable region (VH) comprising an amino acid sequence at least 95% identical to SEQ ID NO: 71; and/or a light chain variable region (VL) comprising an amino acid sequence at least 95% identical to SEQ ID NO: 70;
(iii) a heavy chain variable region (VH) comprising an amino acid sequence at least 95% identical to SEQ ID NO: 72; and/or a light chain variable region (VL) comprising an amino acid sequence at least 95% identical to SEQ ID NO: 70;
(iv) a heavy chain variable region (VH) comprising an amino acid sequence at least 95% identical to SEQ ID NO: 73; and/or a light chain variable region (VL) comprising an amino acid sequence at least 95% identical to SEQ ID NO: 74;
(v) a heavy chain variable region (VH) comprising an amino acid sequence at least 95% identical to SEQ ID NO: 73; and/or a light chain variable region (VL) comprising an amino acid sequence at least 95% identical to SEQ ID NO: 75;
(vi) a heavy chain variable region (VH) comprising an amino acid sequence at least 95% identical to SEQ ID NO: 76; and/or a light chain variable region (VL) comprising an amino acid sequence at least 95% identical to SEQ ID NO: 74;
(vii) a heavy chain variable region (VH) comprising an amino acid sequence at least 95% identical to SEQ ID NO: 69; and/or a light chain variable region (VL) comprising an amino acid sequence at least 95% identical to SEQ ID NO: 70;
(viii) a heavy chain variable region (VH) comprising an amino acid sequence at least 95% identical to SEQ ID NO: 77; and/or a light chain variable region (VL) comprising an amino acid sequence at least 95% identical to SEQ ID NO: 78;
(ix) a heavy chain variable region (VH) comprising an amino acid sequence at least 95% identical to SEQ ID NO: 79; and/or a light chain variable region (VL) comprising an amino acid sequence at least 95% identical to SEQ ID NO: 80; or
(x) a heavy chain variable region (VH) comprising an amino acid sequence at least 95% identical to SEQ ID NO: 77; and/or a light chain variable region (VL) comprising an amino acid sequence at least 95% identical to SEQ ID NO: 80.

In some embodiments, the antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 71 and a VL comprising the amino acid sequence of SEQ ID NO: 70;
(iii) a VH comprising the amino acid sequence of SEQ ID NO: 72 and a VL comprising the amino acid sequence of SEQ ID NO: 70;
(iv) a VH comprising the amino acid sequence of SEQ ID NO: 73 and a VL comprising the amino acid sequence of SEQ ID NO: 74;
(v) a VH comprising the amino acid sequence of SEQ ID NO: 73 and a VL comprising the amino acid sequence of SEQ ID NO: 75;
(vi) a VH comprising the amino acid sequence of SEQ ID NO: 76 and a VL comprising the amino acid sequence of SEQ ID NO: 74;
(vii) a VH comprising the amino acid sequence of SEQ ID NO: 69 and a VL comprising the amino acid sequence of SEQ ID NO: 70;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.

In some embodiments, the antibody is selected from the group consisting of a full-length IgG, a Fab fragment, a Fab′ fragment, a F(ab′)2 fragment, a scFv, and a Fv. In some embodiments, the antibody is a full-length IgG. In some embodiments, the full-length IgG comprises a heavy chain constant region of the isotype IgG1, IgG2, IgG3, or IgG4.

In some embodiments, the antibody comprises:
(i) a heavy chain comprising an amino acid sequence at least 85% identical to SEQ ID NO: 91; and/or a light chain comprising an amino acid sequence at least 85% identical to SEQ ID NO: 90;
(ii) a heavy chain comprising an amino acid sequence at least 85% identical to SEQ ID NO: 86; and/or a light chain comprising an amino acid sequence at least 85% identical to SEQ ID NO: 85;
(iii) a heavy chain comprising an amino acid sequence at least 85% identical to SEQ ID NO: 87; and/or a light chain comprising an amino acid sequence at least 85% identical to SEQ ID NO: 85;
(iv) a heavy chain comprising an amino acid sequence at least 85% identical to SEQ ID NO: 88; and/or a light chain comprising an amino acid sequence at least 85% identical to SEQ ID NO: 89;
(v) a heavy chain comprising an amino acid sequence at least 85% identical to SEQ ID NO: 88; and/or a light chain comprising an amino acid sequence at least 85% identical to SEQ ID NO: 90;
(vi) a heavy chain comprising an amino acid sequence at least 85% identical to SEQ ID NO: 91; and/or a light chain comprising an amino acid sequence at least 85% identical to SEQ ID NO: 89;
(vii) a heavy chain comprising an amino acid sequence at least 85% identical to SEQ ID NO: 84; and/or a light chain comprising an amino acid sequence at least 85% identical to SEQ ID NO: 85;
(viii) a heavy chain comprising an amino acid sequence at least 85% identical to SEQ ID NO: 92; and/or a light chain comprising an amino acid sequence at least 85% identical to SEQ ID NO: 93;
(ix) a heavy chain comprising an amino acid sequence at least 85% identical to SEQ ID NO: 94; and/or a light chain comprising an amino acid sequence at least 85% identical to SEQ ID NO: 95; or
(x) a heavy chain comprising an amino acid sequence at least 85% identical to SEQ ID NO: 92; and/or a light chain comprising an amino acid sequence at least 85% identical to SEQ ID NO: 95.

In some embodiments, the antibody is a Fab fragment.

In some embodiments, the antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 98; and/or a light chain comprising an amino acid sequence at least 85% identical to SEQ ID NO: 85;
(iii) a heavy chain comprising an amino acid sequence at least 85% identical to SEQ ID NO: 99; and/or a light chain comprising an amino acid sequence at least 85% identical to SEQ ID NO: 85;
(iv) a heavy chain comprising an amino acid sequence at least 85% identical to SEQ ID NO: 100; and/or a light chain comprising an amino acid sequence at least 85% identical to SEQ ID NO: 89;
(v) a heavy chain comprising an amino acid sequence at least 85% identical to SEQ ID NO: 100; and/or a light chain comprising an amino acid sequence at least 85% identical to SEQ ID NO: 90;
(vi) a heavy chain comprising an amino acid sequence at least 85% identical to SEQ ID NO: 101; and/or a light chain comprising an amino acid sequence at least 85% identical to SEQ ID NO: 89;
(vii) a heavy chain comprising an amino acid sequence at least 85% identical to SEQ ID NO: 97; and/or a light chain comprising an amino acid sequence at least 85% identical to SEQ ID NO: 85;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.

In some embodiments, the antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 98; and a light chain comprising the amino acid sequence of SEQ ID NO: 85;
(iii) a heavy chain comprising the amino acid sequence of SEQ ID NO: 99; and a light chain comprising the amino acid sequence of SEQ ID NO: 85;
(iv) a heavy chain comprising the amino acid sequence of SEQ ID NO: 100; and a light chain comprising the amino acid sequence of SEQ ID NO: 89;
(v) a heavy chain comprising the amino acid sequence of SEQ ID NO: 100; and a light chain comprising the amino acid sequence of SEQ ID NO: 90;
(vi) a heavy chain comprising the amino acid sequence of SEQ ID NO: 101; and a light chain comprising the amino acid sequence of SEQ ID NO: 89;
(vii) a heavy chain comprising the amino acid sequence of SEQ ID NO: 97; and a light chain comprising the amino acid sequence of SEQ ID NO: 85;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.

In some embodiments, the equilibrium dissociation constant (K_{D}) of binding of the antibody to the transferrin receptor is in a range from 10⁻¹¹ M to 10⁻⁶ M.

In some embodiments, the antibody does not specifically bind to the transferrin binding site of the transferrin receptor and/or wherein the antibody does not inhibit binding of transferrin to the transferrin receptor.

In some embodiments, the antibody is cross-reactive with extracellular epitopes of two or more of a human, non-human primate and rodent transferrin receptor.

In some embodiments, the anti-TfR1 antibody has undergone pyroglutamate formation resulting from a post-translational modification.

In some embodiments, the complex is configured to promote transferrin receptor mediated internalization of the molecular payload into a muscle cell.

In some embodiments, the molecular payload is an oligonucleotide.

In some embodiments, the molecular payload is an oligonucleotide comprising an antisense strand comprising a region of complementarity to an MSTN target sequence. In some embodiments, the MSTN target sequence is an MSTN mRNA sequence as set forth in SEQ ID NOs: 146-148, or an MSTN target sequence as set forth in any one of SEQ ID NOs: 149-196. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 197-220, wherein each of the Us are optionally and independently Ts. In some embodiments, the antisense strand comprises the nucleotide sequence of any one of SEQ ID NOs: 197-220, wherein each of the Us are optionally and independently Ts.

In some embodiments, wherein the molecular payload is an oligonucleotide comprising an antisense strand comprising a region of complementarity to an INHBA target sequence. In some embodiments, the INHBA target sequence is an INHBA mRNA sequence as set forth in SEQ ID NO: 269 or SEQ ID NO: 270, or an INHBA target sequence as set forth in any one of SEQ ID NOs: 271-318. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 319-342, wherein each of the Us are optionally and independently Ts. In some embodiments, the antisense strand comprises the nucleotide sequence of any one of SEQ ID NOs: 319-342, wherein each of the Us are optionally and independently Ts.

In some embodiments, the molecular payload is an oligonucleotide comprising an antisense strand comprising a region of complementarity to an ACVR1B target sequence. In some embodiments, the ACVR1B target sequence is an ACVR1B mRNA sequence as set forth in any one of SEQ ID NOs: 367-370, or an ACVR1B target sequence as set forth in any one of SEQ ID NOs: 221-268. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 343-366, wherein each of the Us are optionally and independently Ts. In some embodiments, the antisense strand comprises the nucleotide sequence of any one of SEQ ID NOs: 343-366, wherein each of the Us are optionally and independently Ts.

In some embodiments, the molecular payload is an oligonucleotide comprising an antisense strand comprising a region of complementarity to a MLCK1 target sequence. In some embodiments, the MLCK1 target sequence is a MLCK1 mRNA as set forth in SEQ ID NO: 411. In some embodiments, the antisense strand is 18-25 nucleotides in length and/or the region of complementarity is at least 16 nucleosides in length.

In some embodiments, the molecular payload is an oligonucleotide comprising an antisense strand comprising a region of complementarity to a ACVR1 target sequence. In some embodiments, the ACVR1 target sequence is an ACVR1 mRNA sequence as set forth in SEQ ID NO: 429 or SEQ ID NO: 430, or an ACVR1 target sequence as set forth in any one of SEQ ID NOs: 431-478. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 479-502, wherein each of the Us are optionally and independently Ts. In some embodiments, the antisense strand comprises the nucleotide sequence of any one of SEQ ID NOs: 479-502, wherein each of the Us are optionally and independently Ts.

In some embodiments, the molecular payload is an oligonucleotide comprising an antisense strand comprising a region of complementarity to a FBXO32 target sequence. In some embodiments, the FBXO32 target sequence is an FBXO32 mRNA sequence as set forth in SEQ ID NO: 505 or SEQ ID NO: 506, or a FBXO32 target sequence as set forth in any one of SEQ ID NOs: 507-554. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 555-578, wherein each of the Us are optionally and independently Ts. In some embodiments, the antisense strand comprises the nucleotide sequence of any one of SEQ ID NOs: 555-578, wherein each of the Us are optionally and independently Ts.

In some embodiments, the molecular payload is an oligonucleotide comprising an antisense strand comprising a region of complementarity to TRIM63 target sequence. In some embodiments, the TRIM63 target sequence is a TRIM63 mRNA sequence as set forth in SEQ ID NO: 579 or SEQ ID NO: 580, or a TRIM63 target sequence as set forth in any one of SEQ ID NOs: 581-628. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 629-652, wherein each of the Us are optionally and independently Ts. In some embodiments, the antisense strand comprises the nucleotide sequence of any one of SEQ ID NOs: 629-652, wherein each of the Us are optionally and independently Ts.

In some embodiments, the molecular payload is an oligonucleotide comprising an antisense strand comprising a region of complementarity to a MEF2D target sequence. In some embodiments, the MEF2D target sequence is an MEF2D mRNA sequence as set forth in SEQ ID NO: 664 or SEQ ID NO: 665, or a MEF2D target sequence as set forth in any one of SEQ ID NOs: 668-715. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 716-223, wherein each of the Us are optionally and independently Ts. In some embodiments, the antisense strand comprises the nucleotide sequence of any one of SEQ ID NOs: 716-223, wherein each of the Us are optionally and independently Ts.

In some embodiments, the molecular payload is an oligonucleotide comprising an antisense strand comprising a region of complementarity to KLF15 target sequence. In some embodiments, the KLF15 target sequence is a KLF15 mRNA sequence as set forth in SEQ ID NO: 740 or SEQ ID NO: 741, or a KLF15 target sequence as set forth in any one of SEQ ID NOs: 742-789. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 790-813, wherein each of the Us are optionally and independently Ts. In some embodiments, the antisense strand comprises the nucleotide sequence of any one of SEQ ID NOs: 790-813, wherein each of the Us are optionally and independently Ts.

In some embodiments, the molecular payload is an oligonucleotide comprising an antisense strand comprising a region of complementarity to a MED1 target sequence. In some embodiments, the MED1 target sequence is a MED1 mRNA sequence as set forth in SEQ ID NO: 814 or SEQ ID NO: 815, or a MED1 target sequence as set forth in any one of SEQ ID NOs: 816-863. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 864-887, wherein each of the Us are optionally and independently Ts. In some embodiments, the antisense strand comprises the nucleotide sequence of any one of SEQ ID NOs: 864-887, wherein each of the Us are optionally and independently Ts.

In some embodiments, the molecular payload is an oligonucleotide comprising an antisense strand comprising a region of complementarity to a MED13 target sequence. In some embodiments, the MED13 target sequence is a MED13 mRNA sequence as set forth in SEQ ID NO: 888 or SEQ ID NO: 889, or a MED13 target sequence as set forth in any one of SEQ ID NOs: 890-937. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 938-961, wherein each of the Us are optionally and independently Ts. In some embodiments, the antisense strand comprises the nucleotide sequence of any one of SEQ ID NOs: 938-961, wherein each of the Us are optionally and independently Ts.

In some embodiments, the molecular payload is an oligonucleotide comprising an antisense strand comprising a region of complementarity to PPP1R3A target sequence. In some embodiments, the PPP1R3A target sequence is a PPP1R3A mRNA sequence as set forth in SEQ ID NO: 962 or SEQ ID NO: 963, or a PPP1R3A target sequence as set forth in any one of SEQ ID NOs: 964-1011. In some embodiments, the antisense strand is 18-25 nucleotides in length and/or the region of complementarity is at least 16 nucleosides in length. In some embodiments, the antisense strand comprises at least 16 consecutive nucleotides of a nucleotide sequence set forth in any one of SEQ ID NOs: 1012-1035, wherein each of the Us are optionally and independently Ts. In some embodiments, the antisense strand comprises the nucleotide sequence of any one of SEQ ID NOs: 1012-1035, wherein each of the Us are optionally and independently Ts.

In some embodiments, the oligonucleotide further comprises a sense strand that hybridizes to the antisense strand to form a double stranded siRNA.

In some embodiments, the oligonucleotide comprises one or more modified nucleosides. In some embodiments, each nucleoside in the oligonucleotide is a modified nucleoside. In some embodiments, the one or more modified nucleosides are 2′ modified nucleotides. In some embodiments, the one or more 2′ modified nucleosides are selected from: 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethyl-bridged nucleic acid (cEt). In some embodiments, the 2′ modified nucleotide is 2′-O-methyl or 2′-fluoro (2′-F). In some embodiments, the oligonucleotide comprises one or more phosphorothioate internucleoside linkages. In some embodiments, the one or more phosphorothioate internucleoside linkage are present on the antisense strand of the RNAi oligonucleotide. In some embodiments, the two internucleoside linkages at the 3′ end of the sense strands are phosphorothioate internucleoside linkages.

In some embodiments, the oligonucleotide is an siRNA listed in Table 10, Table 13, Table 16, Table 19, Table 22, Table 25, Table 28, Table 31, Table 34, Table 37, or Table 40.

In some embodiments, the antibody is covalently linked to the molecular payload via: (i) a cleavable linker; or (ii) a non-cleavable linker. In some embodiments, the cleavable linker comprises a valine-citrulline sequence. In some embodiments, the non-cleavable linker is an alkane linker.

Other aspects of the present disclosure provide methods of reducing MSTN, INHBA, ACVR1B, MLCK1, ACVR1, FBXO32, TRIM63, MEF2D, KLF15, MED1, MED13, and/or PPP1R3A expression in a muscle cell, the method comprising contacting the muscle cell with an effective amount of the complex described herein for promoting internalization of the molecular payload to the muscle cell.

Other aspects of the present disclosure provide methods of treating muscle atrophy the method comprising administering to a subject in need thereof an effective amount of the complex described herein, wherein the subject has elevated expression or activity of MSTN, INHBA, and/or ACVR1B, and the complex comprises a molecular payload that modulates the expression or activity of MSTN, INHBA, and/or ACVR1B. In some embodiments, the subject is a human. In some embodiments, the administration in intravenous.

Other aspects of the present disclosure provide methods of treating irritable bowel syndrome (IBS) or irritable bowel disease (IBD) the method comprising administering to a subject in need thereof an effective amount of the complex described herein, wherein the subject has elevated levels of MLCK1 protein and the complex comprises a molecular payload that modulates the expression or activity of MLCK1. In some embodiments, the subject is a human. In some embodiments, the administration in intravenous.

Other aspects of the present disclosure provide methods of treating a disease associated with an elevated level of ACVR1, the method comprising administering to a subject in need thereof an effective amount of the complex described herein, wherein the subject has elevated levels of ACVR1 protein and the complex comprises a molecular payload that modulates the expression or activity of ACVR1. In some embodiments, the disease associated with an elevated level of ACVR1 is muscle atrophy. In some embodiments, the muscle atrophy is sarcopenia or cachexia. In some embodiments, the subject is a human. In some embodiments, the administration in intravenous.

Other aspects of the present disclosure provide methods of treating muscle atrophy the method comprising administering to a subject in need thereof an effective amount of the complex described herein, wherein the subject has elevated expression or activity of FBXO32 or TRIM63, and the complex comprises a molecular payload that modulates the expression or activity of FBXO32 or TRIM63. In some embodiments, the subject is a human. In some embodiments, the administration in intravenous.

Other aspects of the present disclosure provide methods of treating a heart disease, the method comprising administering to a subject in need thereof an effective amount of the complex described herein, wherein the subject has elevated expression or activity of MEF2D, KLF15, MED1, MED13, and/or PPP1R3A, and the complex comprises a molecular payload that modulates the expression or activity of MEF2D, KLF15, MED1, MED13, and/or PPP1R3A. In some embodiments, the subject is a human. In some embodiments, the administration in intravenous.

In some embodiments, the complex reduces RNA level of MSTN, INHBA, ACVR1B, MLCK1, ACVR1, FBXO32, TRIM63, MEF2D, KLF15, MED1, MED13, and/or PPP1R3A. In some embodiments, the complex reduces protein level of MSTN, INHBA, ACVR1B, MLCK1, ACVR1, FBXO32, TRIM63, MEF2D, KLF15, MED1, MED13, and/or PPP1R3A.

### BRIEF DESCRIPTION OF THE DRAWINGS

FIG. 1 depicts a non-limiting schematic showing the effect of transfecting cells with an siRNA.

FIG. 2 depicts a non-limiting schematic showing the activity of a muscle targeting complex comprising an siRNA.

FIGS. 3A-3B depict non-limiting schematics showing the activity of a muscle targeting complex comprising an siRNA in mouse muscle tissue (gastrocnemius (FIG. 3A) and heart (FIG. 3B)) in vivo, relative to vehicle-treated control experiments. (N=4 C57BL/6 WT mice).

FIGS. 4A-4E depict non-limiting schematics showing the tissue selectivity of a muscle targeting complex (anti-TfR1 antibody-siHPRT) comprising an anti-TfR Fab (RI7 217) conjugated to HPRT-specific siRNA to reduce expression levels of HPRT genes. The data show gene expression in brain (FIG. 4A), liver (FIG. 4B), lung (FIG. 4C), kidney (FIG. 4D), and spleen (FIG. 4E), and demonstrate that muscle targeting complexes do not facilitate gene inhibition in non-muscle tissues.

FIG. 5 shows inhibition of MSTN gene expression by 24 siRNAs tested at 0.5 nM and 10 nM doses.

FIG. 6 shows dose response curves for inhibition of human MSTN by oligonucleotide candidate sequences over a range of concentrations from 100 nM to 10 fM.

FIG. 7 shows inhibition of INHBA gene expression by 24 siRNAs tested at 0.5 nM and 10 nM doses

FIG. 8 shows dose response curves for inhibition of human INHBA by oligonucleotide candidate sequences over a range of concentrations from 100 nM to 10 fM.

FIG. 9 shows inhibition of ACVR1B gene expression by 24 siRNAs tested at 0.1 nM and 10 nM doses.

FIG. 10 shows dose response curves for inhibition of human ACVR1B by oligonucleotide candidate sequences over a range of concentrations from 100 nM to 10 fM.

FIG. 11 shows dose response curves for inhibition of murine ACVR1B by oligonucleotide candidate sequences over a range of concentrations from 100 nM to 10 fM.

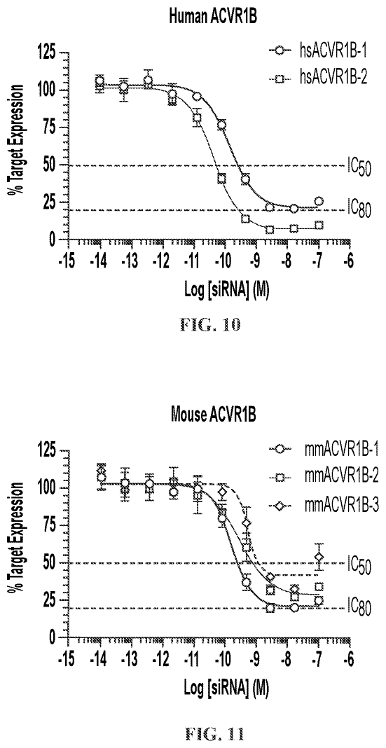

FIG. 12 shows percent knockdown of human ACVR1 expression by oligonucleotide candidate sequences in cell culture at concentrations of 10 nM and 0.5 nM.

FIG. 13 shows dose response curves for inhibition of human ACVR1 by different oligonucleotides over a range of concentrations from 100 nM to 10 fM.

FIG. 14 depicts the results of a dual-luciferase gene inhibition assay used to identify candidate oligonucleotides capable of inhibiting expression of FBXO32. Candidate oligonucleotides (siRNA molecules) were evaluated at a concentration of 0.1 nM and a concentration of 10 nM in a cross species, human/cyno, or rat/mouse system.

FIG. 15 depicts the results of a dual-luciferase gene inhibition assay used to identify candidate oligonucleotides capable of inhibiting expression of TRIM63. Candidate oligonucleotides (siRNA molecules) were evaluated at a concentration of 0.1 nM and a concentration of 10 nM in a cross species, human/cyno, or rat/mouse system.

FIGS. 16A-16B depict a dose response curve for inhibition of human (FIG. 16A) and murine (FIG. 16B) FBXO32 by candidate oligonucleotide sequences over a range of concentrations from 100 nM to 10 fM.

FIGS. 17A-17B depict a dose response curve for inhibition of human (FIG. 17A) and murine (FIG. 17B) TRIM63 by candidate oligonucleotide sequences over a range of concentrations from 100 nM to 10 fM.

FIG. 18 shows percent knockdown of human and murine MEF2D expression by oligonucleotide candidate sequences in cell culture at concentrations of 10 nM and 0.1 nM.

FIG. 19 shows percent knockdown of human and murine KLF15 expression by oligonucleotide candidate sequences in cell culture at concentrations of 10 nM and 0.1 nM.

FIG. 20 shows percent knockdown of human MED1 expression by oligonucleotide candidate sequences in cell culture at concentrations of 10 nM and 0.5 nM.

FIG. 21 shows percent knockdown of human MED13 expression by oligonucleotide candidate sequences in cell culture at concentrations of 10 nM and 0.1 nM.

FIG. 22 shows percent knockdown of human and murine PPP1R3A expression by oligonucleotide candidate sequences in cell culture at concentrations of 10 nM and 0.5 nM.

FIGS. 23A-23B show dose response curves for inhibition of human MEF2D (FIG. 23A) and murine MEF2D (FIG. 23B) by oligonucleotide candidate sequences over a range of concentrations from 100 nM to 10 fM.

FIGS. 24A-24B show dose response curves for inhibition of human KLF15 (FIG. 24A) and murine KLF15 (FIG. 24B) by oligonucleotide candidate sequences over a range of concentrations from 100 nM to 10 fM.

FIG. 25 shows dose response curves for inhibition of human MED1 by oligonucleotide candidate sequences over a range of concentrations from 100 nM to 10 fM.

FIG. 26 shows dose response curves for inhibition of human MED13 by oligonucleotide candidate sequences over a range of concentrations from 100 nM to 10 fM.

FIGS. 27A-27B show dose response curves for inhibition of human PPP1R3A (FIG. 27A) and murine PPP1R3A (FIG. 27B) by oligonucleotide candidate sequences over a range of concentrations from 100 nM to 10 fM.

FIGS. 28A-28H show that conjugates having an anti-TfR1 Fab conjugated to a DMPK-targeting oligonucleotide (AS0300) reduced mouse DMPK expression in various muscle tissues in a mouse model that expresses human TfR1. The DMPK-targeting oligonucleotide was conjugated to anti-TfR1 Fab 3M12-VH4/VK3. FIG. 28A shows that the conjugate reduced mouse wild-type Dmpk in Tibialis Anterior by 79%. FIG. 28B shows that the conjugate reduced mouse wild-type Dmpk in gastrocnemius by 76%. FIG. 28C shows that the conjugate reduced mouse wild-type Dmpk in the heart by 70%. FIG. 28D shows that the conjugate reduced mouse wild-type Dmpk and in diaphragm by 88%. FIGS. 28E-28H show oligonucleotide distributions in Tibialis Anterior (FIG. 28E), gastrocnemius (FIG. 28F), heart (FIG. 28G), and diaphragm (FIG. 28H).

### DETAILED DESCRIPTION

Some aspects of the present disclosure provide molecular payloads (e.g., oligonucleotides) that modulate the expression or activity of genes (e.g., MSTN, INHBA, or ACDR1B) associated with muscle health (e.g., muscle growth and maintenance). Other aspects of the disclosure relate to a recognition that while certain molecular payloads (e.g., oligonucleotides, peptides, small molecules) can have beneficial effects in muscle cells (e.g., cardiac muscle cells), it has proven challenging to effectively target such cells. Accordingly, further provided herein are complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having a rare muscle disease. In some embodiments, complexes provided herein are designed to target cardiac muscle cells or cardiac muscle tissues. In some embodiments, complexes provided herein are provided for treating subjects having muscle atrophy (e.g., sarcopenia or cachexia). For example, in some embodiments, complexes are provided for targeting MSTN expression to treat subjects having cardiac muscle wasting, cardiomyopathy, or cardiac cachexia, and/or skeletal muscle atrophy. In some embodiments, complexes are provided for targeting INHBA to treat subjects having muscle atrophy (e.g., cardiac muscle atrophy). In some embodiments, complexes are provided for targeting ACVR1B to treat subjects having cardiac fibrosis or cardiac hypertrophy.

Myostatin, also referred to as growth differentiation factor 8 (GDF8), is a secreted growth factor that negatively regulates muscle mass. In humans, myostatin is encoded by the MSTN gene. Loss-of-function mutations in the Myostatin gene (MSTN), leading to a hypermuscular phenotype, have been described in cattle, sheep, fish, dogs and humans. Myostatin is expressed in skeletal muscle, with lower levels of expression reported in adipose and cardiac tissues. Inhibition of Myostatin signaling leads to an increase in muscle size.

Myostatin may inhibit cardiomyocyte proliferation and differentiation by manipulating cell cycle progression, and has been shown to prevent cell cycle G1 to S phase transition by decreasing levels of cyclin-dependent kinase complex 2 (CDK2) and by increasing p21 levels. Physiologically, minimal amounts of cardiac myostatin are secreted from the myocardium into serum, having a limited effect on muscle growth. However, increases in cardiac myostatin can increase its serum concentration, which may cause skeletal muscle atrophy.

Pathological states that increase cardiac stress and promote heart failure can induce a rise in both cardiac myostatin mRNA and protein levels within the heart. In ischemic or dilated cardiomyopathy, increased levels of myostatin mRNA have been detected within the left ventricle. Furthermore, increases in myostatin levels during chronic heart failure have been shown to cause cardiac cachexia. It has been shown that systemic inhibition of cardiac myostatin maintains overall muscle weight in experimental models with pre-existing heart failure.

Inhibin beta A (INHBA) is a protein that can exist as an oligomer subunit of activin A and inhibin A. In some instances, INHBA can form a disulfide-linked homodimer (i.e., dimer between two INHBA molecules) to form activin A, which enhances follicle-stimulating hormone (FSH) biosynthesis and secretion, and is involved in several biological processes including cell proliferation and differentiation, immune response and wound repair, and endocrine function. In other instances, INHBA can dimerize with inhibin alpha to form inhibin A, which decreases FSH biosynthesis and secretion.

Activin A interacts with Activin type 1 receptors (e.g., ACVR1, ACVR1B, and ACVR1C) and Activin type 2 receptors (ACVR2A and ACVR2B). These protein-protein interactions lead to phosphorylation of SMAD2 and SMAD3, which can ultimately result in the changes in gene expression for a large variety of genes.

Activin A has been shown to negatively regulate muscle mass (e.g., in connection with myostatin) and thus has been implicated in several muscle disorders, including muscle atrophy (e.g., cardiac muscle atrophy), e.g., as described in Lee S J, et al., “Regulation of muscle mass by follistatin and activins”, Mol. Endocrinol. 2010 Oct.; 24(10):1998-2008; and Lach-Trifilieff et al., Mol Cell Biol. 2014 February; 34(4): 606-618. In some instances, muscle atrophy results in life threatening complications. Elevated Activin A level has also been associated with myocardial complications in type 2 diabetes patients (e.g., as described in Lin et al., Acta Cardiol Sin. 2016 July; 32(4): 420-427; and Kuo et al., Sci Rep 8, 9957 (2018)). These indications demonstrate that compositions and methods for targeting activin A and its subunit INHBA could provide therapeutic benefit. However, effective treatments that target the function and expression of INHBA (e.g., including dimerization to form activin A) are limited.

Activin receptor type-1B (ACVR1B), also known as ALK-4, is a transmembrane serine/threonine kinase activin type-1 receptor that interacts with activin receptor type-2 to form an activin receptor complex. The activin receptor complex functions to bind to activin and regulate a diverse array of cellular processes through signal transduction, including neuronal differentiation and survival, wound healing, extracellular matrix production, immunosuppression and carcinogenesis. Within the receptor complex, ACVR1B becomes phosphorylated by activin receptor type-2 proteins following activin binding. Phosphorylated ACVR1B can subsequently phosphorylate several of the SMAD proteins (e.g., SMAD2 and SMAD3) to propagate activin signaling. An interaction between ACVR1B and SMAD7 can alternatively function to inhibit activin signaling.

It has been established that activin, functioning through its signal transduction pathway through ACVR1B, is a key regulator of cardiac fibrosis (e.g., atrial fibrosis). This regulation is thought to be enhanced by presence of Angiotensin-II. Cardiac fibrosis, a condition involving excess production of extracellular matrix in the cardiac muscle, is commonly associated with structural remodeling associated with abnormal cardiac function, atrial fibrillation, and/or heart attacks. See, e.g., Wang, Q. et al. “The crucial role of activin A/ALK4 pathway in the pathogenesis of Ang-II-induced atrial fibrosis and vulnerability to atrial fibrillation.” Basic Res Cardiol. 2017 July; 112(4):47, the content of which is incorporated herein by reference. It has further been shown that targeting ACVR1B functions to counteract cardiac fibrosis and dysfunction in subjects having cardiac fibrosis. Additionally, inhibition of ACVR1B has an effect in subjects having cardiac hypertrophy. See, e.g., Chen Y. H. et al., “Haplodeficiency of activin receptor-like kinase 4 alleviates myocardial infarction-induced cardiac fibrosis and preserves cardiac function.” J Mol Cell Cardiol. 2017 April; 105:1-11.; and Wang, Q. et al., “Activin Receptor-Like Kinase 4 Haplodeficiency Mitigates Arrhythmogenic Atrial Remodeling and Vulnerability to Atrial Fibrillation in Cardiac Pathological Hypertrophy.” J Am Heart Assoc. 2018 Aug. 21; 7(16):e008842; the contents of each of which are incorporated herein by reference.

Some aspects of the present disclosure provide molecular payloads that modulate the expression or activity of MLCK1 (e.g., oligonucleotides targeting MLCK1 RNAs). Other aspects of the disclosure relate to a recognition that while certain molecular payloads (e.g., oligonucleotides, peptides, small molecules) can have beneficial effects in muscle cells, it has proven challenging to effectively target such cells. Accordingly, further provided herein are complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having a rare muscle disease. In some embodiments, complexes provided herein are designed to target smooth muscle cells or smooth muscle tissues. For example, in some embodiments, complexes are provided for targeting a MLCK1 to treat subjects having irritable bowel syndrome (IBS) or inflammatory bowel disease (IBD).

Myosin light chain kinase (“MLCK1” or “MYLK”), also known as kinase-related protein or telokin, is an enzyme that phosphorylates myosin regulatory light chains in order to facilitate myosin interaction with actin filaments in smooth muscle. MLCK1 is one of four isoforms of myosin light chain kinase and is expressed in smooth muscle. The other isoforms—MLCK2, MLCK3, and MLCK4—are expressed in skeletal, cardiac, and cancerous cells, respectively.

It has recently been shown that MLCK1 is a potential therapeutic target for irritable bowel syndrome (See, Graham, W. V. et al. “Intracellular MLCK1 diversion reverses barrier loss to restore mucosal homeostasis.” Nature Medicine, volume 25, 690-700, 2019). MLCK1 is a critical protein in regulating epithelial barrier dysfunction, which is associated with intestinal diseases (e.g., irritable bowel syndrome). Restoration of the epithelial barrier in smooth muscles tissues (e.g., through inhibition of MLCK1) can limit or reverse these intestinal diseases. Thus, development of novel MLCK1 inhibitors is desired.

Some aspects of the present disclosure provide molecular payloads that modulate the expression or activity of ACVR1 (e.g., oligonucleotides targeting ACVR1 RNAs). Other aspects of the disclosure relate to a recognition that while certain molecular payloads (e.g., oligonucleotides, peptides, small molecules) can have beneficial effects in muscle cells, it has proven challenging to effectively target such cells. Accordingly, provided herein are complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having a rare muscle disease. In some embodiments, complexes provided herein are designed to target cardiac muscle cells or cardiac muscle tissues. For example, in some embodiments, complexes are provided for targeting an ACVR1 to treat subjects having cardiac disease (e.g., cardiac hypertrophy) or muscle atrophy (e.g., sarcopenia or cachexia).

Activin A receptor, type 1 (ACVR1), a BMP type I receptor (also known as Activin receptor-like kinase-2 (ALK-2), ACTRIA, ACVRLK2), is a signaling receptor that binds to Activin A. These protein-protein interactions lead to phosphorylation of SMAD2 and SMAD3, which can ultimately result in the changes in gene expression for a large variety of genes.

ACVR1 has been associated with angiotensin II-induced cardiac hypertrophy and muscle atrophy (e.g., sarcopenia or cachexia). Specifically, deletion of ACVR1 in cardiomyocytes has been shown to reduce cardiac hypertrophy in diseased mice (Shahid, M. et al. “BMP type I receptor ALK2 is required for angiotensin II-induced cardiac hypertrophy” Am J Physiol Heart Circ Physiol. 2016 Apr. 15; 310(8):H984-94). Fibrodysplasia ossificans progressiva (FOP) is caused by heterozygous mutations in ACVR1 (e.g., ACVR1 R206H mutation). These indications demonstrate that compositions and methods for targeting ACVR1 could provide therapeutic benefit. However, effective treatments that target the function and expression of ACVR1 are limited.

Some aspects of the present disclosure provide molecular payloads (e.g., oligonucleotides) that modulate the expression or activity of genes associated with muscle atrophy (e.g., FBXO32 or TRIM63). Other aspects of the disclosure relate to a recognition that while certain molecular payloads (e.g., oligonucleotides, peptides, small molecules) can have beneficial effects in muscle cells, it has proven challenging to effectively target such cells. Accordingly, further provided herein are complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having a rare muscle disease. In some embodiments, complexes provided herein are designed to target cardiac muscle cells or cardiac muscle tissues. For example, in some embodiments, complexes are provided for targeting a FBXO32 to treat subjects having muscle atrophy. In some embodiments, complexes are provided for targeting a TRIM63 to treat subjects having muscle atrophy.

FBXO32, which is also referred to as atrogin-1 and Muscle atrophy F-box gene (MAFbx), is an E3 ubiquitin ligase and a member of the F-box protein family. F-box proteins have been shown to regulate ubiquitin-mediated protein degradation. Although FBXO32 lacks leucine-rich regions and WD40 repeats that are commonly found in F-box proteins, FBXO32 comprises a PDZ domain that is capable of binding other proteins. Serving as an adaptor, FBXO32 bridges proteins to be ubiquitinated with other components of the Skp, Cullin, F-box containing complex (or SCF complex). In humans, FBXO32 protein is encoded by the FBXO32 gene.

FBXO32 is predominantly expressed in striated muscle and has been implicated in regulating protein synthesis and degradation during muscle atrophy. For example, FBXO32 expression is significantly increased during muscle atrophy. See, e.g., Gomes et al., Proc Natl Acad Sci USA. 2001 Dec. 4; 98(25):14440-5. Notably, FBXO32 has been shown to be required for muscle atrophy that is induced by a variety of conditions. For example, in animal models, FBXO32 deficiency prevented muscle atrophy caused by denervation. Small hairpin RNAs (shRNAs) targeting FBXO32 blocked muscle loss induced by fasting in mice. Knockout of FBXO32 also prevented glucocorticoid treatment-induced muscle atrophy. Whereas wild-type mice treated with the synthetic glucocorticoid dexamethasone had decreased wet weight of the triceps surae and tibialis anterior muscles, FBXO32 knockout mice had no muscle sparing. FBXO32 is also a biomarker for cancer cachexia. Furthermore, knockout of FBXO32 prevented myostatin-induced growth inhibition in primary myoblasts. See, e.g., Bodine et al., Science. 2001 Nov. 23; 294(5547):1704-8; Cong et al., Hum Gene Ther. 2011 March; 22(3):313-24; Baehr et al., J Physiol. 2011 Oct. 1; 589(Pt 19):4759-76; and Lokireddy et al., Am J Physiol Cell Physiol 303: C512-C529, 2012; Sukari et al., Semin Cancer Biol. 2016 February; 36:95-104; Wang et al., Diabetes. 2010 August; 59(8):1879-89. Further, it has been shown that FBXO32 disrupts Akt-dependent pathways responsible for physiologic cardiac hypertrophy (see, e.g., Li et al., J Clin Invest. 2007 Nov. 1; 117(11): 3211-3223). Overexpression of FBXO32 in cardiac muscle may afford therapeutic values for cardiac hypertrophy.

TRIM63 is a member of the RING finger protein family and may be referred to as Muscle-specific RING finger protein 1 (MuRF1). Like FBXO32, TRIM63 is a E3 ubiquitin ligase that is predominantly expressed in muscle, including skeletal, cardiac, and smooth muscle, and the iris. For example, TRIM63 may be detected in the M-line and Z-line lattices of myofibrils.

Several studies have implicated TRIM63 in muscle atrophy. For example, TRIM63 has been shown to be required for skeletal muscle atrophy. Mice that were deficient in TRIM63 did not develop muscle atrophy. See, e.g., Bodine et al., Science. 2001 Nov. 23; 294(5547):1704-8. Whereas wild-type mice showed significant muscle atrophy when treated with a synthetic glucocorticoid (dexamethasone), TRIM63 null mice showed muscle sparing. Knockout of TRIM63 may maintain protein synthesis in mice, suggesting that TRIM63 is capable of regulating cellular protein levels in a proteasome-independent manner. See, e.g., Bodine et al., J Physiol. 2011 Oct. 1; 589(Pt 19):4759-76. TRIM63 has been shown to degrade myosin heavy chain protein under dexamethasone-induced atrophy conditions and mice with knockout of TRIM63 show less myosin heavy chain protein degradation than wild-type mice. See, e.g., Clarke et al., Cell Metab. 2007 November; 6(5):376-85. Similarly, muscles lose myosin-binding protein C (MyBP-C) and myosin light chains 1 and 2 (MyLC1 and MyLC2) from myofibrils when muscle atrophy is induced by denervation or fasting. Loss of MyBP-C, MyLC1, and MyLC2 occur in a TRIM63-dependent manner. See, e.g., Cohen et al., J Cell Biol. 2009 Jun. 15; 185(6):1083-95. miRNA-based short hairpin RNAs (shRNAs) targeting TRIM63 and genetic knockout of TRIM63 have also been used to determine the role of TRIM63 in acute lung injury-associated skeletal muscle atrophy. TRIM63 deficiency attenuated muscle wasting induced by acute lung injury. See, e.g., Files et al., Am J Respir Crit Care Med. 2012 Apr. 15; 185(8):825-34.

Some aspects of the present disclosure relate to a recognition that while certain molecular payloads (e.g., oligonucleotides, peptides, small molecules) can have beneficial effects in muscle cells, it has proven challenging to effectively target such cells. As described herein, the present disclosure provides complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having a rare muscle disease. In some embodiments, complexes provided herein are designed to target cardiac muscle cells or cardiac muscle tissues. For example, in some embodiments, complexes are provided for targeting a MEF2D, KLF15, MED1, MED13, or PPP1R3A gene to treat subjects having a muscular disease or a heart disease.

MEF2D is a member of the myocyte-specific enhancer factor 2 (MEF2) family of transcription factors. Alternative splicing MEF2D mRNA results in multiple transcript variants, a ubiquitous isoform and a tissue-specific isoform primarily detected in muscle tissue.

Kruppel-like factor 15 (KLF15) is a protein that belongs to the Kruppel family of transcription factors and can function as either a repressor or activator of gene transcription. Expression levels of KLF15 are increased by glucocorticoid signaling and blood levels of insulin. In muscle tissues, levels of KLF15 increase in response to exercise and control the ability of muscle tissue to burn fat and generate force. KLF15 specifically interacts with MEF2 and synergistically activates the GLUT4 promoter via an intact KLF15-binding site proximal to the MEF2A site. miR-133 targets KLF15 in cardiac and skeletal muscles to regulate the expression of GLUT4. KLF15 inhibits cardiac hypertrophy by repressing the activity of MEF2 and other cardiac transcription factors (e.g., GATA4 and myocardin). Expression levels of KLF15 are reduced in failing human hearts and in human aortic aneurysm tissues. Accordingly, KLF15 is involved in metabolic control in cardiomyocytes and skeletal muscle tissues and is a therapeutic target for cardiac diseases such as cardiac hypertrophy and cardiac failure (e.g., following a myocardial infarction; see, e.g., Zhao, Y. et al., “Multiple roles of KLF15 in the heart: Underlying mechanisms and therapeutic implications.” J Mol Cell Cardiol. 2019 Apr; 129:193-196; the contents of which are incorporated herein by reference in its entirety). KLF15 expression levels also impacts how potassium flows out of heart cells. It has been shown that elevated or reduced levels of KLF15 may result in heart arrhythmias.

The Mediator (MED) complex is regulator of eukaryotic gene transcription. Recent studies have further demonstrated that several subunits of the MED complex including MED1, MED13, MED14, MED15, MED23, MED25 and CDK8 play important regulatory roles in metabolism (e.g., glucose and lipid metabolism). In part due to their import in metabolism, some of these subunits (e.g., MED1 and MED13) have been linked to cardiovascular diseases (e.g., human congenital heart diseases). However, targeting MED subunits (e.g., MED1 and MED13) with small molecule inhibitors has proven challenging. New methods and compositions for targeting the Mediator complex (e.g., subunits such as MED1 and MED13), e.g., for treating cardiovascular diseases, are needed.

The glycogen-associated form of protein phosphatase-1 (PP1) derived from skeletal muscle is a heterodimer composed of a 37-kDa catalytic subunit (OMIM entry 176875) and a 124-kDa targeting and regulatory subunit, referred to as protein phosphatase 1 regulatory subunit 3A (PPP1R3A). PPP1R3A binds to muscle glycogen with high affinity and enhances dephosphorylation of glycogen-bound substrates for PP1 such as glycogen synthase and glycogen phosphorylase kinase. PPP1R3A is a central regulator in heart failure and is implicated in cardiomyocyte metabolic pathways.

Further aspects of the disclosure, including a description of defined terms, are provided below.

### I. Definitions

ACVR1: As used herein, the term “ACVR1”, “ALK2”, or “ALK-2” refers to a gene that encodes activin A receptor type 1, a protein receptor involved in the bone morphogenesis among other functions. In some embodiments, ACVR1 may be a human (Gene ID: 90) (e.g., SEQ ID NO: 429), non-human primate (e.g., Gene ID: 697935 (e.g., SEQ ID NO: 423), Gene ID: 470565 (e.g., SEQ ID NO: 424), Gene ID: 102134051 (e.g., SEQ ID NO: 425)), or rodent gene (e.g., Gene ID: 11477 (e.g., SEQ ID NO: 430), Gene ID: 79558 (e.g., SEQ ID NO: 426)). In humans, several genetic mutations in the gene that lead to alterations in the ACVR1 protein, e.g., L196P, R202I, R206H, Q207E, G328R, G328W, G328E, G356D, R375P, AP197-F198, are associated with FOP (e.g., as described in Haupt et al., Bone. 2018 April; 109: 232-240). In addition, multiple human transcript variants (e.g., as annotated under GenBank RefSeq Accession Numbers: NM_001105.5 (SEQ ID NO: 429), NM_001111067.4 (SEQ ID NO: 427), NM_001347663.1 (SEQ ID NO: 217), NM_001347664.1 (SEQ ID NO: 218), NM_001347665.1 (SEQ ID NO: 219), NM_001347666.1 (SEQ ID NO: 220), and NM_001347667.2 (SEQ ID NO: 221)) have been characterized that encode different protein isoforms. An exemplary ACVR1 protein, encoded by a human ACVR1 gene, is annotated under NCBI Reference Sequence: NP 001096.1, and has the following amino acid sequence:

(SEQ ID NO: 428)
MVDGVMILPVLIMIALPSPSMEDEKPKVNPKLYMCVCEGLSCGNEDHCE
GQQCFSSLSINDGFHVYQKGCFQVYEQGKMTCKTPPSPGQAVECCQGDW
CNRNITAQLPTKGKSFPGTQNFHLEVGLIILSVVFAVCLLACLLGVALR
KFKRRNQERLNPRDVEYGTIEGLITTNVGDSTLADLLDHSCTSGSGSGL
PFLVQRTVARQITLLECVGKGRYGEVWRGSWQGENVAVKIFSSRDEKSW
FRETELYNTVMLRHENILGFIASDMTSRHSSTQLWLITHYHEMGSLYDY
LQLTTLDTVSCLRIVLSIASGLAHLHIEIFGTQGKPAIAHRDLKSKNIL
VKKNGQCCIADLGLAVMHSQSTNQLDVGNNPRVGTKRYMAPEVLDETIQ
VDCFDSYKRVDIWAFGLVLWEVARRMVSNGIVEDYKPPFYDVVPNDPSF
EDMRKVVCVDQQRPNIPNRWFSDPTLTSLAKLMKECWYQNPSARLTALR
IKKTLTKIDNSLDKLKTDC

ACVR1B: As used herein, the term, “ACVR1B” or “ALK-4” refers to a gene that encodes activin A receptor type 1B. ACVR1B is a transmembrane serine/threonine kinase activin type-1 receptor that interacts with activin receptor type-2 to form an activin receptor complex to enable activin signaling. In some embodiments, ACVR1B may be a human (Gene ID: 91) (e.g., SEQ ID NOs: 367-368), non-human primate (e.g., Gene ID: 696587 (e.g., SEQ ID NO: 384), Gene ID: 101865702 (e.g., SEQ ID NO: 385)), or rodent gene (e.g., Gene ID: 11479 (e.g., SEQ ID NO: 369), Gene ID: 29381 (e.g., SEQ ID NO: 370)). In addition, multiple exemplary human transcripts (e.g., as annotated under GenBank RefSeq Accession Number: NM_004302.5 (SEQ ID NO: 367), NM_020327.3 (SEQ ID NO: 386), NM_020328.4 (SEQ ID NO: 387), XM_017020201.2 (SEQ ID NO: 388), XM_011538966.3 (SEQ ID NO: 389), and XM_011538967.3 (SEQ ID NO: 390)) have been characterized. Exemplary ACVR1B proteins, encoded by a human ACVR1B gene, are annotated under NCBI Reference Sequences: NP_004293.1 (SEQ ID NO: 142), NP_064732.3 (SEQ ID NO: 143), and NP_064733.3 (SEQ ID NO: 144), and have the following amino acid sequences:

NP_004293.1
(SEQ ID NO: 142)
MAESAGASSFFPLVVLLLAGSGGSGPRGVQALLCACTSCLQANYTCETD
GACMVSIFNLDGMEHHVRTCIPKVELVPAGKPFYCLSSEDLRNTHCCYT
DYCNRIDLRVPSGHLKEPEHPSMWGPVELVGIIAGPVFLLFLIIIIVFL
VINYHQRVYHNRQRLDMEDPSCEMCLSKDKTLQDLVYDLSTSGSGSGLP
LFVQRTVARTIVLQEIIGKGRFGEVWRGRWRGGDVAVKIFSSREERSWF
REAEIYQTVMLRHENILGFIAADNKDNGTWTQLWLVSDYHEHGSLFDYL
NRYTVTIEGMIKLALSAASGLAHLHMEIVGTQGKPGIAHRDLKSKNILV
KKNGMCAIADLGLAVRHDAVTDTIDIAPNQRVGTKRYMAPEVLDETINM
KHFDSFKCADIYALGLVYWEIARRCNSGGVHEEYQLPYYDLVPSDPSIE
EMRKVVCDQKLRPNIPNWWQSYEALRVMGKMMRECWYANGAARLTALRI
KKTLSQLSVQEDVKI
NP_064732.3
(SEQ ID NO: 143)
MVSIFNLDGMEHHVRTCIPKVELVPAGKPFYCLSSEDLRNTHCCYTDYC
NRIDLRVPSGHLKEPEHPSMWGPVELVGIIAGPVFLLFLIIIIVFLVIN
YHQRVYHNRQRLDMEDPSCEMCLSKDKTLQDLVYDLSTSGSGSGLPLFV
QRTVARTIVLQEIIGKGRFGEVWRGRWRGGDVAVKIFSSREERSWFREA
EIYQTVMLRHENILGFIAADNKDNGTWTQLWLVSDYHEHGSLFDYLNRY
TVTIEGMIKLALSAASGLAHLHMEIVGTQGKPGIAHRDLKSKNILVKKN
GMCAIADLGLAVRHDAVIDTIDIAPNQRVGTKRYMAPEVLDETINMKHF
DSFKCADIYALGLVYWEIARRCNSGGVHEEYQLPYYDLVPSDPSIEEMR
KVVCDQKLRPNIPNWWQSYEALRVMGKMMRECWYANGAARLTALRIKKT
LSQLSVQEDVKI
NP_064733.3
(SEQ ID NO: 144)
MAESAGASSFFPLVVLLLAGSGGSGPRGVQALLCACTSCLQANYTCETD
GACMVSIFNLDGMEHHVRTCIPKVELVPAGKPFYCLSSEDLRNTHCCYT
DYCNRIDLRVPSGHLKEPEHPSMWGPVELVGIIAGPVFLLFLIIIIVFL
VINYHQRVYHNRQRLDMEDPSCEMCLSKDKTLQDLVYDLSTSGSGSGLP
LFVQRTVARTIVLQEIIGKGRFGEVWRGRWRGGDVAVKIFSSREERSWF
REAEIYQTVMLRHENILGFIAADNKADCSFLTLPWEVVMVSAAPKLRSL
RLQYKGGRGRARFLFPLNNGTWTQLWLVSDYHEHGSLFDYLNRYTVTIE
GMIKLALSAASGLAHLHMEIVGTQGKPGIAHRDLKSKNILVKKNGMCAI
ADLGLAVRHDAVTDTIDIAPNQRVGTKRYMAPEVLDETINMKHFDSFKC
ADIYALGLVYWEIARRCNSGGVHEEYQLPYYDLVPSDPSIEEMRKVVCD
QKLRPNIPNWWQSYEALRVMGKMMRECWYANGAARLTALRIKKTLSQLS
VQEDVKI

Administering: As used herein, the terms “administering” or “administration” means to provide a complex to a subject in a manner that is physiologically and/or pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (A), epsilon (e), gamma (γ) or mu (p) heavy chain. In some embodiments, the heavy chain of an antibody described herein can comprise a human alpha (a), delta (A), epsilon (e), gamma (γ) or mu (p) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, A1-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Disease allele: As used herein, the term “disease allele” refers to any one of alternative forms (e.g., mutant forms) of a gene, such as, but not limited to, a MLCK1 gene, an ACVR1 gene, or a FBXO32 gene, for which the allele is correlated with and/or directly or indirectly contributes to, or causes, disease. A disease allele may comprise gene alterations including, but not limited to, insertions, deletions, missense mutations, nonsense mutations and splice-site mutations relative to a wild-type (non-disease) allele. In some embodiments, a disease allele has a loss-of-function mutation. In some embodiments, a disease allele has a gain-of-function mutation. In some embodiments, a disease allele encodes an activating mutation (e.g., encodes a protein that is constitutively active). In some embodiments, a disease allele is a recessive allele having a recessive phenotype. In some embodiments, a disease allele is a dominant allele having a dominant phenotype. In some embodiments, a disease allele has a loss-of-function mutation in a gene encoding MLCK1 (MYLK). In some embodiments, a loss-of-function mutation is as described in Halim D. et al. “Loss-of-Function Variants in MYLK Cause Recessive Megacystis Microcolon Intestinal Hypoperistalsis Syndrome.” Am J Hum Genet. 2017 Jul. 6; 101(1):123-129; Hannuksela M. et al. “A novel variant in MYLK causes thoracic aortic dissections: genotypic and phenotypic description.” BMC Med Genet. 2016 Sep. 1; 17(1):61; or Shalata, A. et al. “Fatal thoracic aortic aneurysm and dissection in a large family with a novel MYLK gene mutation: delineation of the clinical phenotype.” Orphanet J Rare Dis. 2018 Mar. 15; 13(1):41; the contents of each of which are incorporated herein by reference. In some embodiments, a disease allele has a gain-of-function mutation. In some embodiments, a disease allele encodes an activating mutation (e.g., encodes a protein that is constitutively active). In some embodiments, a disease allele is a recessive allele having a recessive phenotype. In some embodiments, a disease allele is a dominant allele having a dominant phenotype. In some embodiments, a disease allele comprises a duplication (e.g., a 7 base pair duplication (c.3838_3844dupGAAAGCG)), a splice-site variant (e.g., c.3985+5C>A), a deletion (e.g., a 2-bp deletion (c3272_3273del, p.Ser1091*)), or a missense mutation (e.g., a missense mutation at c.4471G>T (Ala1491Ser)). In some embodiments, a disease allele may comprise one or more deletions or substitutions that lead to alterations in the ACVR1 protein, e.g., L196P, R202I, R206H, Q207E, G328R, G328W, G328E, G356D, R375P, AP197-F198. In some embodiments, a subject may have Fibrodysplasia ossificans progressiva (FOP). In some embodiments, a subject having FOP may have one or two mutated ACVR1 alleles. In some embodiments, a subject having classic or typical FOP has an ACVR1 allele comprising a mutation that leads to R206H ACVR1 protein. In some embodiments, a subject having atypical FOP has an ACVR1 allele comprising at least one mutation that leads to mutated ACVR1 protein that does not comprise the R206H mutation. In some embodiments, a diseased allele of MEF2D is an isoform of MEF2D lacking the j-exon and is associated with muscle degeneration disorders such as myotonic dystrophy (e.g., as described in Lee et al., The Journal of Biological Chemistry, 285, 33779-33787, 2010, incorporated herein by reference). In some embodiments, a disease allele of MED13 comprises a missense mutation. In some embodiments a disease allele of MED13 encodes a T326I, P327S and/or P327Q mutation. In some embodiments, a disease allele of MED13 comprises an in-frame deletion (e.g., of nucleotides encoding T326). In some embodiments, the disease MED13 allele is as described in Snijders Blok L., et. al, “De novo mutations in MED13, a component of the Mediator complex, are associated with a novel neurodevelopmental disorder” Hum. Genet. 2018, 137:375-388.; the contents of which are incorporated herein by reference.

FBXO32: As used herein, the term, “FBXO32,” refers to a gene that encodes a F-box adaptor protein ad is a member of SKP1-cullin-F-box (SCF) ubiquitin protein ligase complex. FBXO32 can bind substrates for ubiquitination by the SCF complex. In some embodiments, FBXO32 may be a human (Gene ID: 114907 (e.g., SEQ ID NO: 505).), non-human primate (e.g., Gene ID: 102141240 (e.g., SEQ ID NO: 653)), or rodent gene (e.g., Gene ID: 67731 (e.g., SEQ ID NO: 506), Gene ID: 171043 (e.g., SEQ ID NO: 654)). In addition, exemplary human transcripts (e.g., as annotated under GenBank RefSeq Accession Number: NM_058229.4 (SEQ ID NO: 505), NM_001242463.2 (SEQ ID NO: 655), and NM_148177.2 (SEQ ID NO: 656)) have been characterized. An exemplary FBXO32 protein, encoded by a human FBXO32 gene, is annotated under NCBI Reference Sequence: NP_478136.1, and has the following amino acid sequence:

(SEQ ID NO: 503)
MPFLGQDWRSPGQNWVKTADGWKRFLDEKSGSFVSDLSSYCNKEVYNKE
NLFNSLNYDVAAKKRKKDMLNSKTKTQYFHQEKWIYVHKGSTKERHGYC
TLGEAFNRLDFSTAILDSRRFNYVVRLLELIAKSQLTSLSGIAQKNFMN
ILEKVVLKVLEDQQNIRLIRELLQTLYTSLCTLVQRVGKSVLVGNINMW
VYRMETILHWQQQLNNIQITRPAFKGLTFTDLPLCLQLNIMQRLSDGRD
LVSLGQAAPDLHVLSEDRLLWKKLCQYHFSERQIRKRLILSDKGQLDWK
KMYFKLVRCYPRKEQYGDTLQLCKHCHILSWKGTDHPCTANNPESCSVS
LSPQDFINLFKF.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-transferrin receptor 1 antibodies and antigen binding portions are provided. Such antibodies may be generated by obtaining murine anti-transferrin receptor 1 monoclonal antibodies using traditional hybridoma technology followed by humanization using in vitro genetic engineering, such as those disclosed in Kasaian et al PCT publication No. WO 2005/123126 A2.

INHBA: As used herein, the term, “INHBA” or “inhibin, beta A” refers to a gene that encodes inhibin, beta A (INHBA). In some embodiments, an INHBA gene may be a human INHBA gene (Gene ID: 3624 (e.g., SEQ ID NO: 269)), non-human primate INHBA gene (e.g., Gene ID: 102146142 (e.g., SEQ ID NO: 391), Gene ID: 702734 (e.g., SEQ ID NO: 392)), or rodent INHBA gene (e.g., Gene ID: 16323 (e.g., SEQ ID NO: 270), Gene ID: 29200 (e.g., SEQ ID NO: 393)). In addition, an exemplary human transcript (e.g., as annotated under GenBank RefSeq Accession Number: NM_002192.4 (SEQ ID NO: 269)) has been characterized. An exemplary INHBA protein, encoded by a human INHBA gene, is annotated under NCBI Reference Sequence: NP_002183.1, and has the following amino acid sequence:

(SEQ ID NO: 145)
MPLLWLRGFLLASCWIIVRSSPTPGSEGHSAAPDCPSCALAALPKDVPN
SQPEMVEAVKKHILNMLHLKKRPDVTQPVPKAALLNAIRKLHVGKVGEN
GYVEIEDDIGRRAEMNELMEQTSEIITFAESGTARKTLHFEISKEGSDL
SVVERAEVWLFLKVPKANRTRTKVTIRLFQQQKHPQGSLDTGEEAEEVG
LKGERSELLLSEKVVDARKSTWHVFPVSSSIQRLLDQGKSSLDVRIACE
QCQESGASLVLLGKKKKKEEEGEGKKKGGGEGGAGADEEKEQSHRPFLM
LQARQSEDHPHRRRRRGLECDGKVNICCKKQFFVSFKDIGWNDWIIAPS
GYHANYCEGECPSHIAGTSGSSLSFHSTVINHYRMRGHSPFANLKSCCV
PTKLRPMSMLYYDDGQNIIKKDIQNMIVEECGCS

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

KLF15: As used herein, the term, “KLF15,” refers to a gene that encodes Kruppel-like factor 15 protein, a transcription factor that, in cardiac and skeletal muscle cells, functions to inhibit the activity of MEF2 and other cardiac transcription factors (e.g., GATA4 and myocardin). In some embodiments, KLF15 refers to a human KLF15 (Gene ID: 28999 (e.g., SEQ ID NO: 1045)), a non-human primate KLF15 (e.g., Gene ID: 716386 (e.g., SEQ ID NO: 1046), Gene ID: 470911 (e.g., SEQ ID NO: 1047)), or rodent KLF15 (e.g., Gene ID: 66277 (e.g., SEQ ID NO: 1048), Gene ID: 85497 (e.g., SEQ ID NO: 1049)). In addition, multiple human KLF15 transcript variants (e.g., as annotated under GenBank RefSeq Accession Numbers: NM_014079.4 (SEQ ID NO: 740), XM_011512743.2 (SEQ ID NO: 1050), and XM_005247400.4 (SEQ ID NO: 1051)) have been characterized that encode different protein isoforms. An exemplary KLF15 protein isoform, encoded by a human KLF15 gene, is annotated under NCBI Reference Sequence: NP_054798.1, and has the following amino acid sequence:

(SEQ ID NO: 131)
MVDHLLPVDENFSSPKCPVGYLGDRLVGRRAYHMLPSPVSEDDSDASSP
CSCSSPDSQALCSCYGGGLGTESQDSILDFLLSQATLGSGGGSGSSIGA
SSGPVAWGPWRRAAAPVKGEHFCLPEFPLGDPDDVPRPFQPTLEEIEEF
LEENMEPGVKEVPEGNSKDLDACSQLSAGPHKSHLHPGSSGRERCSPPP
GGASAGGAQGPGGGPTPDGPIPVLLQIQPVPVKQESGTGPASPGQAPEN
VKVAQLLVNIQGQTFALVPQVVPSSNLNLPSKFVRIAPVPIAAKPVGSG
PLGPGPAGLLMGQKFPKNPAAELIKMHKCTFPGCSKMYTKSSHLKAHLR
RHTGEKPFACTWPGCGWRFSRSDELSRHRRSHSGVKPYQCPVCEKKFAR
SDHLSKHIKVHRFPRSSRSVRSVN

Mediator (MED) complex subunit: As used herein, the term “Mediator complex subunit” or “subunit of the Mediator complex” refers to an individual component of the Mediator complex. Subunits of the Mediator complex include MED1, MED13, MED14, MED15, MED23, MED25, CDK8, and others. Simple eukaryotes (e.g., Saccharomyces cerevisiae (yeast)) commonly have up to 21 MED complex subunits; while mammals typically have between 26 and 31 MED complex subunits.

MED1: As used herein, the term, “MED1,” generally refers to a gene that encodes Mediator complex subunit 1 (MED1). In some embodiments, MED1 may be a human (Gene ID: 5469 (e.g., SEQ ID NO: 1052)), non-human primate (e.g., Gene ID: 101925389 (e.g., SEQ ID NO: 1053), Gene ID: 697781 (e.g., SEQ ID NO: 1054)), or rodent gene (e.g., Gene ID: 19014 (e.g., SEQ ID NO: 1055), Gene ID: 497991 (e.g., SEQ ID NO: 1056)). In addition, an exemplary human transcript (e.g., as annotated under GenBank RefSeq Accession Number: NM_004774.4 (SEQ ID NO: 814)) has been characterized. An exemplary MED1 protein, encoded by a human MED1 gene, is annotated under NCBI Reference Sequence: NP_004765.2; and has the following amino acid sequence:

(SEQ ID NO: 138)
MKAQGETEESEKLSKMSSLLERLHAKFNQNRPWSETIKLVRQVMEKRVV
MSSGGHQHLVSCLETLQKALKVTSLPAMTDRLESIARQNGLGSHLSASG
TECYITSDMFYVEVQLDPAGQLCDVKVAHHGENPVSCPELVQQLREKNF
DEFSKHLKGLVNLYNLPGDNKLKTKMYLALQSLEQDLSKMAIMYWKATN
AGPLDKILHGSVGYLTPRSGGHLMNLKYYVSPSDLLDDKTASPIILHEN
NVSRSLGMNASVTIEGTSAVYKLPIAPLIMGSHPVDNKWTPSFSSITSA
NSVDLPACFFLKFPQPIPVSRAFVQKLQNCTGIPLFETQPTYAPLYELI
TQFELSKDPDPIPLNHNMRFYAALPGQQHCYFLNKDAPLPDGRSLQGTL
VSKITFQHPGRVPLILNLIRHQVAYNTLIGSCVKRTILKEDSPGLLQFE
VCPLSESRFSVSFQHPVNDSLVCVVMDVQDSTHVSCKLYKGLSDALICT
DDFIAKVVQRCMSIPVTMRAIRRKAETIQADTPALSLIAETVEDMVKKN
LPPASSPGYGMTTGNNPMSGTTTPTNTFPGGPITTLFNMSMSIKDRHES
VGHGEDFSKVSQNPILTSLLQITGNGGSTIGSSPTPPHHTPPPVSSMAG
NTKNHPMLMNLLKDNPAQDFSTLYGSSPLERQNSSSGSPRMEICSGSNK
TKKKKSSRLPPEKPKHQTEDDFQRELFSMDVDSQNPIFDVNMTADTLDT
PHITPAPSQCSTPPTTYPQPVPHPQPSIQRMVRLSSSDSIGPDVTDILS
DIAEEASKLPSTSDDCPAIGTPLRDSSSSGHSQSTLFDSDVFQTNNNEN
PYTDPADLIADAAGSPSSDSPTNHFFHDGVDFNPDLLNSQSQSGFGEEY
FDESSQSGDNDDFKGFASQALNTLGVPMLGGDNGETKFKGNNQADTVDF
SIISVAGKALAPADLMEHHSGSQGPLLTTGDLGKEKTQKRVKEGNGTSN
STLSGPGLDSKPGKRSRTPSNDGKSKDKPPKRKKADTEGKSPSHSSSNR
PFTPPTSTGGSKSPGSAGRSQTPPGVATPPIPKITIQIPKGTVMVGKPS
SHSQYTSSGSVSSSGSKSHHSHSSSSSSSASTSGKMKSSKSEGSSSSKL
SSSMYSSQGSSGSSQSKNSSQSGGKPGSSPITKHGLSSGSSSTKMKPQG
KPSSLMNPSLSKPNISPSHSRPPGGSDKLASPMKPVPGTPPSSKAKSPI
SSGSGGSHMSGTSSSSGMKSSSGLGSSGSLSQKTPPSSNSCTASSSSFS
SSGSSMSSSQNQHGSSKGKSPSRNKKPSLTAVIDKLKHGVVTSGPGGED
PLDGQMGVSTNSSSHPMSSKHNMSGGEFQGKREKSDKDKSKVSTSGSSV
DSSKKTSESKNVGSTGVAKIIISKHDGGSPSIKAKVTLQKPGESSGEGL
RPQMASSKNYGSPLISGSTPKHERGSPSHSKSPAYTPQNLDSESESGSS
IAEKSYQNSPSSDDGIRPLPEYSTEKHKKHKKEKKKVKDKDRDRDRDKD
RDKKKSHSIKPESWSKSPISSDQSLSMTSNTILSADRPSRLSPDFMIGE
EDDDLMDVALIGN

MED13: As used herein, the term, “MED13” or “PROSIT240” generally refers PGP-27 DNA to a gene that encodes Mediator complex subunit 13 (MED13). MED13 is one component of a four-subunit kinase module of the Mediator complex that further comprises cyclin C, cyclin-dependent kinase 8 (CDK8), and MED12. In some embodiments, MED13 may be a human (Gene ID: 9969 (e.g., SEQ ID NO: 1057)), non-human primate (e.g., Gene ID: 712277 (e.g., SEQ ID NO: 1058), Gene ID: 102120434 (e.g., SEQ ID NO: 1059)), or rodent gene (e.g., Gene ID: 327987 (e.g., SEQ ID NO: 1060), Gene ID: 303403 (e.g., SEQ ID NO: 1061)). In addition, an exemplary human transcript of MED13 (e.g., as annotated under GenBank RefSeq Accession Number: NM_005121.3 (SEQ ID NO: 888)) has been characterized. An exemplary MED13 protein, encoded by a human MED13 gene, is annotated under NCBI Reference Sequence: NP_005112.2; and has the following amino acid sequence:

(SEQ ID NO: 139)
MSASFVPNGASLEDCHCNLFCLADLTGIKWKKYVWQGPTSAPILFPVTE
EDPILSSFSRCLKADVLGVWRRDQRPGRRELWIFWWGEDPSFADLIHHD
LSEEEDGVWENGLSYECRTLLFKAVHNLLERCLMNRNFVRIGKWFVKPY
EKDEKPINKSEHLSCSFTFFLHGDSNVCTSVEINQHQPVYLLSEEHITL
AQQSNSPFQVILCPFGLNGTLTGQAFKMSDSATKKLIGEWKQFYPISCC
LKEMSEEKQEDMDWEDDSLAAVEVLVAGVRMIYPACFVLVPQSDIPTPS
PVGSTHCSSSCLGVHQVPASTRDPAMSSVTLTPPTSPEEVQTVDPQSVQ
KWVKFSSVSDGFNSDSTSHHGGKIPRKLANHVVDRVWQECNMNRAQNKR
KYSASSGGLCEEATAAKVASWDFVEATQRTNCSCLRHKNLKSRNAGQQG
QAPSLGQQQQILPKHKTNEKQEKSEKPQKRPLTPFHHRVSVSDDVGMDA
DSASQRLVISAPDSQVRFSNIRTNDVAKTPQMHGTEMANSPQPPPLSPH
PCDVVDEGVTKTPSTPQSQHFYQMPTPDPLVPSKPMEDRIDSLSQSFPP
QYQEAVEPTVYVGTAVNLEEDEANIAWKYYKFPKKKDVEFLPPQLPSDK
FKDDPVGPFGQESVTSVTELMVQCKKPLKVSDELVQQYQIKNQCLSAIA
SDAEQEPKIDPYAFVEGDEEFLFPDKKDRQNSEREAGKKHKVEDGTSSV
TVLSHEEDAMSLFSPSIKQDAPRPTSHARPPSTSLIYDSDLAVSYTDLD
NLFNSDEDELTPGSKKSANGSDDKASCKESKTGNLDPLSCISTADLHKM
YPTPPSLEQHIMGFSPMNMNNKEYGSMDTTPGGTVLEGNSSSIGAQFKI
EVDEGFCSPKPSEIKDFSYVYKPENCQILVGCSMFAPLKTLPSQYLPPI
KLPEECIYRQSWTVGKLELLSSGPSMPFIKEGDGSNMDQEYGTAYTPQT
HTSFGMPPSSAPPSNSGAGILPSPSTPRFPTPRTPRTPRTPRGAGGPAS
AQGSVKYENSDLYSPASTPSTCRPLNSVEPATVPSIPEAHSLYVNLILS
ESVMNLFKDCNFDSCCICVCNMNIKGADVGVYIPDPTQEAQYRCTCGFS
AVMNRKFGNNSGLFLEDELDIIGRNTDCGKEAEKRFEALRATSAEHVNG
GLKESEKLSDDLILLLQDQCTNLFSPFGAADQDPFPKSGVISNWVRVEE
RDCCNDCYLALEHGRQFMDNMSGGKVDEALVKSSCLHPWSKRNDVSMQC
SQDILRMLLSLQPVLQDAIQKKRTVRPWGVQGPLTWQQFHKMAGRGSYG
TDESPEPLPIPTFLLGYDYDYLVLSPFALPYWERLMLEPYGSQRDIAYV
VLCPENEALLNGAKSFFRDLTAIYESCRLGQHRPVSRLLTDGIMRVGST
ASKKLSEKLVAEWFSQAADGNNEAFSKLKLYAQVCRYDLGPYLASLPLD
SSLLSQPNLVAPTSQSLITPPQMTNTGNANTPSATLASAASSTMTVTSG
VAISTSVATANSTLTTASTSSSSSSNLNSGVSSNKLPSFPPFGSMNSNA
AGSMSTQANTVQSGQLGGQQTSALQTAGISGESSSLPTQPHPDVSESTM
DRDKVGIPTDGDSHAVTYPPAIVVYIIDPFTYENTDESTNSSSVWTLGL
LRCFLEMVQTLPPHIKSTVSVQIIPCQYLLQPVKHEDREIYPQHLKSLA
FSAFTQCRRPLPTSTNVKTLTGFGPGLAMETALRSPDRPECIRLYAPPF
ILAPVKDKQTELGETFGEAGQKYNVLFVGYCLSHDQRWILASCTDLYGE
LLETCIINIDVPNRARRKKSSARKFGLQKLWEWCLGLVQMSSLPWRVVI
GRLGRIGHGELKDWSCLLSRRNLQSLSKRLKDMCRMCGISAADSPSILS
ACLVAMEPQGSFVIMPDSVSTGSVFGRSTTLNMQTSQLNTPQDTSCTHI
LVFPTSASVQVASATYTTENLDLAFNPNNDGADGMGIFDLLDTGDDLDP
DIINILPASPTGSPVHSPGSHYPHGGDAGKGQSTDRLLSTEPHEEVPNI
LQQPLALGYFVSTAKAGPLPDWFWSACPQAQYQCPLFLKASLHLHVPSV
QSDELLHSKHSHPLDSNQTSDVLRFVLEQYNALSWLTCDPATQDRRSCL
PIHFVVLNQLYNFIMNML

MEF2D: As used herein, the term, “MEF2D,” refers to a gene that encodes myocyte enhancer factor 2D, a member of the myocyte-specific enhancer factor 2 (MEF2) family of transcription factors. MEF2D binds specifically to the MEF2 element, 5′-YTA[AT]4TAR-3′, found in numerous muscle-specific, growth factor and stress induced genes. MEF2D mediates cellular functions not only in skeletal and cardiac muscle development, but also in neuronal differentiation and survival. MEF2D also plays diverse roles in the control of cell growth, survival and apoptosis and in the regulation of neuronal apoptosis. MEF2D has been shown to be play important roles in heart development and in heart diseases (e.g., cardiac hypertrophy, cardiomyopathy), and in muscular diseases (e.g., muscle atrophy, myotonic dystrophy). See e.g., Chen et al., Oncotarget. 2017 Dec. 19; 8(67): 112152-112165, incorporated herein by reference. It has been shown that reducing MEF2D activity in the heart resulted in resistance to cardiac hypertrophy, fetal gene activation, and fibrosis in response to pressure overload and β-chronic adrenergic stimulation in mice, and that overexpression of MEF2D was sufficient to drive the fetal gene program and pathological remodeling of the heart (see, e.g., Kim et al., J Clin Invest. 2008 Jan. 2; 118(1): 124-132, incorporated herein by reference). Additionally, MEF2D is involved in neuromuscular diseases, such as Parkinson's disease (see, e.g., Yao et al., The Journal of Biological Chemistry, 287, 34246-34255, 2012, incorporated herein by reference) and amyotrophic lateral sclerosis (see, e.g., Arosio et al., Molecular and Cellular Neuroscience, Volume 74, July 2016, Pages 10-17, incorporated herein by reference). In some embodiments, MEF2D refers to a human (Gene ID: 4209 (e.g., SEQ ID NO: 664)), a non-human primate (e.g., Gene ID: 102143822 (e.g., SEQ ID NO: 1062), or rodent gene (e.g., Gene ID: 17261 (e.g., SEQ ID NO: 666), Gene ID: 81518 (e.g., SEQ ID NO: 1063)). In addition, multiple human MEF2D transcript variants (e.g., as annotated under GenBank RefSeq Accession Numbers: NM_001271629.2 (SEQ ID NO: 665), NM_005920.4 (SEQ ID NO: 664), XM_006711332.3 (SEQ ID NO: 1036), XM_006711334.3 (SEQ ID NO: 1037), XM_006711333.2 (SEQ ID NO: 1038), XM_005245169.4 (SEQ ID NO: 1039), XM_017001315.1 (SEQ ID NO: 1040), XM_006711330.3 (SEQ ID NO: 1041), XM_005245170.3 (SEQ ID NO: 1042), XM_011509569.3 (SEQ ID NO: 1043), and XM_017001314.1 (SEQ ID NO: 1044)) have been characterized that encode different protein isoforms. Exemplary MEF2D protein isoforms, encoded by a human MEF2D gene, are annotated under NCBI Reference Sequence: NP_001258558.1 and NP_005911.1, and has the following amino acid sequence, respectively:

(SEQ ID NO: 140)
MGRKKIQIQRITDERNRQVTFTKRKFGLMKKAYELSVLCDCEIALIIFN
HSNKLFQYASTDMDKVLLKYTEYNEPHESRTNADIIETLRKKGFNGCDS
PEPDGEDSLEQSPLLEDKYRRASEELDGLFRRYGSTVPAPNFAMPVTVP
VSNQSSLQFSNPSGSLVTPSLVTSSLTDPRLLSPQQPALQRNSVSPGLP
QRPASAGAMLGGDLNSANGACPSPVGNGYVSARASPGLLPVANGNSLNK
VIPAKSPPPPTHSTQLGAPSRKPDLRVITSQAGKGLMHHLNNAQRLGVS
QSTHSLTTPVVSVATPSLLSQGLPFSSMPTAYNTDYQLTSAELSSLPAF
SSPGGLSLGNVTAWQQPQQPQQPQQPQPPQQQPPQPQQPQPQQPQQPQQ
PPQQQSHLVPVSLSNLIPGSPLPHVGAALTVTTHPHISIKSEPVSPSRE
RSPAPPPPAVFPAARPEPGDGLSSPAGGSYETGDRDDGRGDFGPTLGLL
RPAPEPEAEGSAVKRMRLDTWTLK
(SEQ ID NO: 141)
MGRKKIQIQRITDERNRQVTFTKRKFGLMKKAYELSVLCDCEIALIIFN
HSNKLFQYASTDMDKVLLKYTEYNEPHESRTNADIIETLRKKGFNGCDS
PEPDGEDSLEQSPLLEDKYRRASEELDGLFRRYGSTVPAPNFAMPVTVP
VSNQSSLQFSNPSGSLVTPSLVTSSLTDPRLLSPQQPALQRNSVSPGLP
QRPASAGAMLGGDLNSANGACPSPVGNGYVSARASPGLLPVANGNSLNK
VIPAKSPPPPTHSTQLGAPSRKPDLRVITSQAGKGLMHHLTEDHLDLNN
AQRLGVSQSTHSLTTPVVSVATPSLLSQGLPFSSMPTAYNTDYQLTSAE
LSSLPAFSSPGGLSLGNVTAWQQPQQPQQPQQPQPPQQQPPQPQQPQPQ
QPQQPQQPPQQQSHLVPVSLSNLIPGSPLPHVGAALTVTTHPHISIKSE
PVSPSRERSPAPPPPAVFPAARPEPGDGLSSPAGGSYETGDRDDGRGDF
GPTLGLLRPAPEPEAEGSAVKRMRLDTWTLK

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

MLCK1: As used herein, the term, “MLCK1” or “MYLK1” refers to a gene that encodes myosin light chain kinase-1 protein, which is an enzyme that phosphorylates myosin regulatory light chains in order to facilitate myosin interaction with actin filaments in smooth muscle. In some embodiments, MLCK1 may be a human (Gene ID: 4638 (e.g., SEQ ID NO: 412)), non-human primate (e.g., Gene ID: 102130711 (e.g., SEQ ID NO: 413)), or rodent gene (e.g., Gene ID: 107589 (e.g., SEQ ID NO: 414), Gene ID: 288057 (e.g., SEQ ID NO: 415)). In addition, several exemplary human transcripts (e.g., as annotated under GenBank RefSeq Accession Number: NM_001321309.2 (SEQ ID NO: 416), NM_053025.4 (SEQ ID NO: 417), NM_053026.4 (SEQ ID NO: 418), NM_053027.4 (SEQ ID NO: 419), NM_053028.4 (SEQ ID NO: 420), NM_053031.4 (SEQ ID NO: 421), and NM_053032.4 (SEQ ID NO: 422)) has been characterized.

An exemplary MLCK1 protein, encoded by a human MLCK1 gene, is annotated under NCBI Reference Sequence: NP_444253.3, and has the following amino acid sequence:

(SEQ ID NO: 410)
MGDVKLVASSHISKTSLSVDPSRVDSMPLTEAPAFILPPRNLCIKEGAT
AKFEGRVRGYPEPQVTWHRNGQPITSGGRFLLDCGIRGTFSLVIHAVHE
EDRGKYTCEATNGSGARQVTVELTVEGSFAKQLGQPVVSKTLGDRFSAP
AVETRPSIWGECPPKFATKLGRVVVKEGQMGRFSCKITGRPQPQVTWLK
GNVPLQPSARVSVSEKNGMQVLEIHGVNQDDVGVYTCLVVNGSGKASMS
AELSIQGLDSANRSFVRETKATNSDVRKEVINVISKESKLDSLEAAAKS
KNCSSPQRGGSPPWAANSQPQPPRESKLESCKDSPRTAPQTPVLQKTSS
SITLQAARVQPEPRAPGLGVLSPSGEERKRPAPPRPATFPTRQPGLGSQ
DVVSKAANRRIPMEGQRDSAFPKFESKPQSQEVKENQTVKFRCEVSGIP
KPEVAWFLEGTPVRRQEGSIEVYEDAGSHYLCLLKARTRDSGTYSCTAS
NAQGQLSCSWTLQVERLAVMEVAPSFSSVLKDCAVIEGQDFVLQCSVRG
TPVPRITWLLNGQPIQYARSTCEAGVAELHIQDALPEDHGTYTCLAENA
LGQVSCSAWVTVHEKKSSRKSEYLLPVAPSKPTAPIFLQGLSDLKVMDG
SQVTMTVQVSGNPPPEVIWLHNGNEIQESEDFHFEQRGTQHSLCIQEVF
PEDTGTYTCEAWNSAGEVRTQAVLTVQEPHDGTQPWFISKPRSVTASLG
QSVLISCAIAGDPFPTVHWLRDGKALCKDTGHFEVLQNEDVFTLVLKKV
QPWHAGQYEILLKNRVGECSCQVSLMLQNSSARALPRGREPASCEDLCG
GGVGADGGGSDRYGSLRPGWPARGQGWLEEEDGEDVRGVLKRRVETRQH
TEEAIRQQEVEQLDFRDLLGKKVSTKTLSEDDLKEIPAEQMDFRANLQR
QVKPKTVSEEERKVHSPQQVDFRSVLAKKGTSKTPVPEKVPPPKPATPD
FRSVLGGKKKLPAENGSSSAETLNAKAVESSKPLSNAQPSGPLKPVGNA
KPAETLKPMGNAKPAETLKPMGNAKPDENLKSASKEELKKDVKNDVNCK
RGHAGTTDNEKRSESQGTAPAFKQKLQDVHVAEGKKLLLQCQVSSDPPA
TIIWTLNGKTLKTTKFIILSQEGSLCSVSIEKALPEDRGLYKCVAKNDA
GQAECSCQVTVDDAPASENTKAPEMKSRRPKSSLPPVLGTESDATVKKK
PAPKTPPKAAMPPQIIQFPEDQKVRAGESVELFGKVTGTQPITCTWMKF
RKQIQESEHMKVENSENGSKLTILAARQEHCGCYTLLVENKLGSRQAQV
NLTVVDKPDPPAGTPCASDIRSSSLTLSWYGSSYDGGSAVQSYSIEIWD
SANKTWKELATCRSTSFNVQDLLPDHEYKFRVRAINVYGTSEPSQESEL
TTVGEKPEEPKDEVEVSDDDEKEPEVDYRTVTINTEQKVSDFYDIEERL
GSGKFGQVFRLVEKKTRKVWAGKFFKAYSAKEKENIRQEISIMNCLHHP
KLVQCVDAFEEKANIVMVLEIVSGGELFERIIDEDFELTERECIKYMRQ
ISEGVEYIHKQGIVHLDLKPENIMCVNKTGTRIKLIDFGLARRLENAGS
LKVLFGTPEFVAPEVINYEPIGYATDMWSIGVICYILVSGLSPFMGDND
NETLANVTSATWDFDDEAFDEISDDAKDFISNLLKKDMKNRLDCTQCLQ
HPWLMKDTKNMEAKKLSKDRMKKYMARRKWQKTGNAVRAIGRLSSMAMI
SGLSGRKSSTGSPTSPLNAEKLESEEDVSQAFLEAVAEEKPHVKPYFSK
TIRDLEVVEGSAARFDCKIEGYPDPEVVWFKDDQSIRESRHFQIDYDED
GNCSLIISDVCGDDDAKYTCKAVNSLGEATCTAELIVETMEEGEGEGEE
EEE

MSTN: As used herein, the term, “MSTN,” refers to a gene that encodes myostatin a secreted growth factor that negatively regulates muscle mass. In some embodiments, MSTN may be a human (Gene ID: 2660 (e.g., SEQ ID NO: 147)), non-human primate (e.g., Gene ID: 710114 (e.g., SEQ ID NO: 394), Gene ID: 470605 (e.g., SEQ ID NO: 395)), or rodent gene (e.g., Gene ID: 29152 (e.g., SEQ ID NO: 396), Gene ID: 17700 (e.g., SEQ ID NO: 148)). In addition, an exemplary human transcript (e.g., as annotated under GenBank RefSeq Accession Number: NM_005259.3 (SEQ ID NO: 147)) has been characterized. An exemplary myostatin protein, encoded by a human MSTN gene, is annotated under NCBI Reference Sequence: NP_005250.1 and has the following amino acid sequence:

(SEQ ID NO: 146)
MQKLQLCVYIYLFMLIVAGPVDLNENSEQKENVEKEGLCNACTWRQNTK
SSRIEAIKIQILSKLRLETAPNISKDVIRQLLPKAPPLRELIDQYDVQR
DDSSDGSLEDDDYHATTETIITMPTESDFLMQVDGKPKCCFFKFSSKIQ
YNKVVKAQLWIYLRPVETPTTVFVQILRLIKPMKDGTRYTGIRSLKLDM
NPGTGIWQSIDVKTVLQNWLKQPESNLGIEIKALDENGHDLAVTFPGPG
EDGLNPFLEVKVTDTPKRSRRDFGLDCDEHSTESRCCRYPLTVDFEAFG
WDWIIAPKRYKANYCSGECEFVFLQKYPHTHLVHQANPRGSAGPCCTPT
KMSPINMLYFNGKEQIIYGKIPAMVVDRCGCS

Muscle atrophy: As used herein, the term, “muscle atrophy,” refers to a condition characterized by muscle wasting. In some embodiments, muscle atrophy is a highly regulated catabolic process which occurs during periods of disuse and/or in response to systemic inflammation (e.g., cachexia). In some embodiments, muscle atrophy is associated with diminishing muscle mass, reduction in muscle size, and/or reduction in the number of muscle cells in a subject. Conditions, including chronic illnesses (e.g., congestive heart failure, diabetes, cancer, AIDS, and renal disease), severe burns, critical care myopathy, limb denervation, stroke, limb fracture, anorexia, spinal cord injury or other conditions leading to muscle disuse may result in muscle atrophy. In some embodiments, muscle atrophy is caused by cancer cachexia, cardiac cachexia, fasting, diabetes, renal failure, denervation, or glucocorticoid-induced muscle atrophy.

Muscle-targeting agent: As used herein, the term, “muscle-targeting agent,” refers to a molecule that specifically binds to an antigen expressed on muscle cells (e.g., cardiac muscle cells). The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells (e.g., cardiac muscle cells). In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

PPP1R3A: As used herein, the term, “PPP1R3A,” refers to a gene that encodes the regulatory subunit of protein phosphatase-1 (PP1). In some embodiments, this regulatory subunit binds to muscle glycogen with high affinity and enhances dephosphorylation of glycogen-bound substrates for PP1 such as glycogen synthase and glycogen phosphorylase kinase. In some embodiments, PPP1R3A may be a human (Gene ID: 5506 (SEQ ID NO: 1064)), non-human primate (e.g., Gene ID: 703562 (e.g., SEQ ID NO: 1065) (Macaca mulatta)), or rodent gene (e.g., Gene ID: 140491 (e.g., SEQ ID NO: 963) (M. musculus), Gene ID: 500036 (e.g., SEQ ID NO: 1066) (R. norvegicus). In addition, an exemplary human transcript (e.g., as annotated under GenBank RefSeq Accession Number: NM_002711.4 (SEQ ID NO: 962)) has been characterized.

An exemplary PPP1R3A protein, encoded by a human PPP1R3A gene, is annotated under NCBI Reference Sequence: NP_002702.2, and has the following amino acid sequence:

(SEQ ID NO: 663)
MEPSEVPSQISKDNFLEVPNLSDSLCEDEEVTFQPGFSPQPSRRGSDSS
EDIYLDTPSSGTRRVSFADSFGFNLVSVKEFDCWELPSASTTFDLGTDI
FHTEEYVLAPLFDLPSSKEDLMQQLQIQKAILESTESLLGSTSIKGIIR
VLNVSFEKLVYVRMSLDDWQTHYDILAEYVPNSCDGETDQFSFKIVLVP
PYQKDGSKVEFCIRYETSVGTFWSNNNGTNYTFICQKKEQEPEPVKPWK
EVPNRQIKGCLKVKSSKEESSVTSEENNFENPKNTDTYIPTIICSHEDK
EDLEASNRNVKDVNREHDEHNEKELELMINQHLIRTRSTASRDERNTFS
TDPVNFPNKAEGLEKKQIHGEICTDLFQRSLSPSSSAESSVKGDFYCNE
KYSSGDDCTHQPSEETTSNMGEIKPSLGDTSSDELVQLHTGSKEVLDDN
ANPAHGNGTVQIPCPSSDQLMAGNLNKKHEGGAKNIEVKDLGCLRRDFH
SDTSACLKESTEEGSSKEDYYGNGKDDEEQRIYLGVNEKQRKNFQTILH
DQERKMGNPKISVAGIGASNRDLATLLSEHTAIPTRAITADVSHSPRTN
LSWEEAVLTPEHHHLTSEGSALGGITGQVCSSRTGNVLRNDYLFQVEEK
SGGINSEDQDNSPQHKQSWNVLESQGKSRENKTNITEHIKGQTDCEDVW
GKRDNTRSLKATTEELFTCQETVCCELSSLADHGITEKAEAGTAYIIKT
TSESTPESMSAREKAIIAKLPQETARSDRPIEVKETAFDPHEGRNDDSH
YTLCQRDTVGVIYDNDFEKESRLGICNVRVDEMEKEETMSMYNPRKTHD
REKCGTGNITSVEESSWVITEYQKATSKLDLQLGMLPTDKTVFSENRDL
RQVQELSKKTDSDAIVHSAFNSDTNRAPQNSSPFSKHHTEISVSTNEQA
IAVENAVTTMASQPISTKSENICNSTREIQGIEKHPYPESKPEEVSRSS
GIVTSGSRKERCIGQIFQTEEYSVEKSLGPMILINKPLENMEEARHENE
GLVSSGQSLYTSGEKESDSSASTSLPVEESQAQGNESLFSKYTNSKIPY
FLLFLIFLITVYHYDLMIGLTFYVLSLSWLSWEEGRQKESVKKK

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a patient having type 2 diabetes. In some embodiments, the subject is a patient having cancer. In some embodiments, the subject is a human patient who has or is suspected of having heart failure, muscle atrophy (e.g., skeletal and/or cardiac muscle atrophy), muscular dystrophies, cachexia (e.g., cardiac cachexia), muscle hypertrophy, cardiac muscle wasting, and/or cardiomyopathy. In some embodiments, a subject having muscle hypertrophy has at least one mutation in MSTN as in Schuelke, M. et al., “Myostatin Mutation Associated with Gross Muscle Hypertrophy in a Child” N Engl J Med 2004; 350:2682-2688, incorporated herein by reference. In some embodiments, the subject is a patient having type 2 diabetes who is suffering from myocardial complications (e.g., heart failure, cardiac muscle atrophy, cachexia, and/or cardiac muscle hypertrophy). In some embodiments, the subject is a cancer patient suffering from cachexia. In some embodiments, the subject is a human patient who has or is suspected of having cardiac fibrosis or cardiac hypertrophy. In some embodiments, the subject is a human patient who has or is suspected of having angiotensin-II-induced cardiac hypertrophy. In some embodiments, the subject has experienced a myocardial infarction (i.e., heart attack). In some embodiments, the subject is a human patient who has or is suspected of having irritable bowel syndrome (IBS). In some embodiments, the subject is a human patient who has or is suspected of having inflammatory bowel disease (IBD). In some embodiments, the subject is a human patient who has familial thoracic aortic aneurysms and dissections (FTAAD). In some embodiments, the subject is a human patient who has Berdon syndrome (also called “recessive megacystis microcolon intestinal hypoperistalsis syndrome”). In some embodiments, the subject has or is suspected of having cardiac hypertrophy. In some embodiments, the subject has or is suspected of having angiotensin II-induced cardiac hypertrophy. In some embodiments, the subject has muscle atrophy (e.g., cardiac muscle atrophy). In some embodiments, the subject is a human patient who has or is suspected of having typical FOP or atypical FOP. In some embodiments, the subject has at least one ACVR1 allele that comprises one or more deletions or substitutions that lead to alterations in the ACVR1 protein, e.g., L196P, R202I, R206H, Q207E, G328R, G328W, G328E, G356D, R375P, AP197-F198.

TRIM63: As used herein, the term, “TRIM63,” refers to a gene that encodes an E3 ubiquitin ligase that is a member of the RING zinc finger protein family. TRIM63 may also be referred to as IRF; SMRZ; MURF1; MURF2; RNF28; or tripartite motif containing 63. In some embodiments, TRIM63 may be a human (Gene ID: 84676 (e.g., SEQ ID NO: 579)), non-human primate (e.g., Gene ID: 102120812 (e.g., SEQ ID NO: 659)), or rodent gene (e.g., Gene ID: 433766 (e.g., SEQ ID NO: 580), Gene ID: 140939 (e.g., SEQ ID NO: 660)). In addition, an exemplary human transcript (e.g., as annotated under GenBank RefSeq Accession Number: NM_032588.3 (SEQ ID NO: 579)) has been characterized.

An exemplary TRIM63 protein, encoded by a human TRIM63 gene, is annotated under NCBI Reference Sequence: NP_115977.2, and has the following amino acid sequence:

(SEQ ID NO: 504)
MDYKSSLIQDGNPMENLEKQLICPICLEMFTKPVVILPCQHNLCRKCAN
DIFQAANPYWTSRGSSVSMSGGRFRCPTCRHEVIMDRHGVYGLQRNLLV
ENIIDIYKQECSSRPLQKGSHPMCKEHEDEKINIYCLTCEVPTCSMCKV
FGIHKACEVAPLQSVFQGQKTELNNCISMLVAGNDRVQTIITQLEDSRR
VTKENSHQVKEELSQKFDTLYAILDEKKSELLQRITQEQEKKLSFIEAL
IQQYQEQLDKSTKLVETAIQSLDEPGGATFLLTAKQLIKSIVEASKGCQ
LGKTEQGFENMDFFTLDLEHIADALRAIDFGTDEEEEEFIEEEDQEEEE
STEGKEEGHQ.

Transferrin receptor: As used herein, the term, “transferrin receptor (also known as CD71, p90, TFR. or TFR1)” refers to an internalizing cell surface receptor that binds transferrin to facilitate iron uptake by endocytosis. In some embodiments, a transferrin receptor may be of human (NCBI Gene ID 7037 (e.g., SEQ ID NO: 397)), non-human primate (e.g., NCBI Gene ID 711568 (e.g., SEQ ID NO: 398) or NCBI Gene ID 102136007 (e.g., SEQ ID NO: 399)), or rodent (e.g., NCBI Gene ID 22042 (e.g., SEQ ID NO: 400)) origin. In addition, multiple human transcript variants have been characterized that encoded different isoforms of the receptor (e.g., as annotated under GenBank RefSeq Accession Numbers: NP_001121620.1 (SEQ ID NO: 401), NP_003225.2 (SEQ ID NO: 105), NP_001300894.1 (SEQ ID NO: 402), and NP_001300895.1 (SEQ ID NO: 403)).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequence, relative to an unmodified oligonucleotide. Examples of structures of 2′-modified nucleosides are provided below:

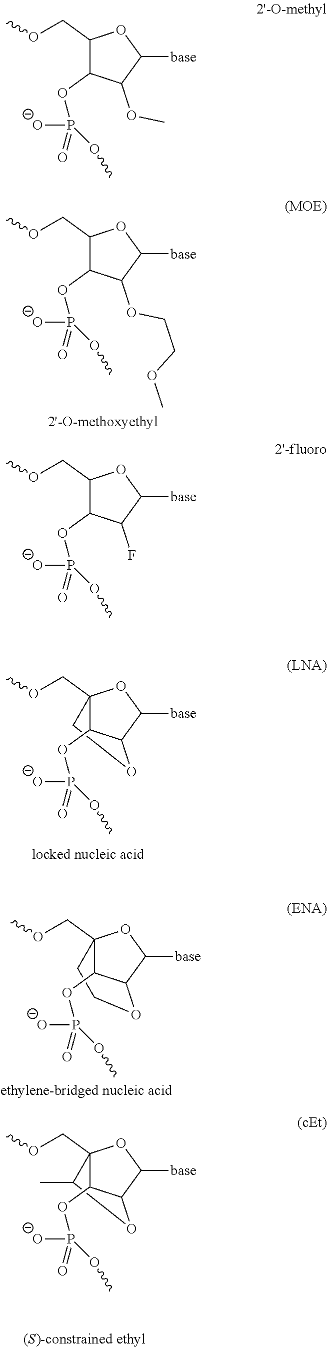

These examples are shown with phosphate groups, but any internucleoside linkages are contemplated between 2′-modified nucleosides.

### II. Complexes

Provided herein are complexes that comprise a targeting agent, e.g., an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or nucleic acid. In some embodiments, the molecular payload present with a complex is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell. In some embodiments, a molecular payload is an oligonucleotide that targets a MSTN gene in muscle cells (e.g., cardiac muscle cells). In some embodiments, a molecular payload is an oligonucleotide that targets INHBA or activin A in muscle cells (e.g., cardiac muscle cells). In some embodiments, a molecular payload is an oligonucleotide that targets ACVR1B in muscle cells (e.g., cardiac muscle cells). In some embodiments, a molecular payload is an oligonucleotide that targets a MLCK1 gene in muscle cells (e.g., smooth muscle cells). In some embodiments, a molecular payload is an oligonucleotide that targets ACVR1 in muscle cells (e.g., cardiac muscle cells). In some embodiments, a molecular payload is an oligonucleotide that targets FBXO32 in muscle cells (e.g., cardiac muscle cells). In some embodiments, a molecular payload is an oligonucleotide that targets TRIM63 in muscle cells (e.g., cardiac muscle cells). In some embodiments, a molecular payload is an oligonucleotide that targets MEF2D, KLF15, MED1, MED13, or PPP1R3A in muscle cells (e.g., cardiac muscle cells). In some embodiments, a molecular payload inhibits the function of MEF2D, KLF15, MED1, MED13, or PPP1R3A in muscle cells. In some embodiments, a molecular payload promotes or enhances the function of MEF2D, KLF15, MED1, MED13, or PPP1R3A in muscle cells (e.g., increases expression).

In some embodiments, a complex comprises a muscle-targeting agent, e.g., an anti-transferrin receptor 1 antibody, covalently linked to a molecular payload, e.g., an antisense oligonucleotide that targets MSTN gene, an antisense oligonucleotide that targets INHBA, or an antisense oligonucleotide that targets ACVR1B. In some embodiments, a complex comprises a muscle-targeting agent, e.g., an anti-transferrin receptor 1 antibody, covalently linked to a molecular payload, e.g., an siRNA oligonucleotide that targets MSTN gene, an antisense oligonucleotide that targets INHBA, or an antisense oligonucleotide that targets ACVR1B. In some embodiments, a complex comprises a muscle-targeting agent, e.g., an anti-transferrin receptor 1 antibody, covalently linked to a molecular payload, e.g., an antisense oligonucleotide or siRNA oligonucleotide that targets MLCK1, ACVR1, FBXO32, TRIM63, MEF2D, KLF15, MED1, MED13, or PPP1R3A.

### A. Muscle-Targeting Agents

Some aspects of the disclosure provide muscle-targeting agents, e.g., for delivering a molecular payload to a muscle cell (e.g., a cardiac muscle cell). In some embodiments, such muscle-targeting agents are capable of binding to a muscle cell, e.g., via specifically binding to an antigen on the muscle cell, and delivering an associated molecular payload to the muscle cell. In some embodiments, muscle-targeting agents are designed to target cardiac muscle cells or cardiac muscle tissues. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure, and that any muscle targets (e.g., muscle surface proteins) can be targeted by any type of muscle target agents described herein. For example, the muscle-targeting agent may comprise, or consist of, a small molecule, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a cardiac muscle cell, a skeletal muscle cell, and/or a smooth muscle cell. In some embodiments, any of the muscle-targeting agents provided herein bind to (e.g., specifically bind to) an antigen on a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-transferrin receptor 1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 Mar, 39(13):78309; the entire contents of each of which are incorporated herein by reference.

a. Anti-Transferrin Receptor 1 Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor 1 antibody, or an anti-TfR1 antibody. Antibodies that bind, e.g., specifically bind, to a transferrin receptor may be internalized into the cell, e.g., through receptor-mediated endocytosis, upon binding to a transferrin receptor.

It should be appreciated that anti-transferrin receptor 1 antibodies may be produced, synthesized, and/or (e.g., and) derivatized using several known methodologies, e.g., library design using phage display. Exemplary methodologies have been characterized in the art and are incorporated by reference (Diez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-transferrin receptor 1 antibody has been previously characterized or disclosed. Antibodies that specifically bind to transferrin receptor are known in the art (see, e.g., U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

Provided herein, in some aspects, are new anti-TfR1 antibodies for use as the muscle targeting agents (e.g., in muscle targeting complexes). In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor.

In some embodiments, the anti-TfR1 antibody described herein (e.g., 3M12 in Table 1 below and its humanized variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 1 below and its humanized variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 1 below and its humanized variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 1 below and its humanized variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 106)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEEENTD
NNTKANGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMESDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 108)
MMDQARSAFSNLFGGEPLSYTRFSLARQVDGDNSHVEMKLAADEEENAD
NNMKASVRKPKRFNGRLCFAAIALVIFFLIGFMSGYLGYCKRVEQKEEC
VKLAETEETDKSETMETEDVPTSSRLYWADLKTLLSEKLNSIEFADTIK
QLSQNTYTPREAGSQKDESLAYYIENQFHEFKFSKVWRDEHYVKIQVKS
SIGQNMVTIVQSNGNLDPVESPEGYVAFSKPTEVSGKLVHANFGTKKDF
EELSYSVNGSLVIVRAGEITFAEKVANAQSFNAIGVLIYMDKNKFPVVE
ADLALFGHAHLGTGDPYTPGFPSFNHTQFPPSQSSGLPNIPVQTISRAA
AEKLFGKMEGSCPARWNIDSSCKLELSQNQNVKLIVKNVLKERRILNIF
GVIKGYEEPDRYVVVGAQRDALGAGVAAKSSVGTGLLLKLAQVFSDMIS
KDGFRPSRSIIFASWTAGDFGAVGATEWLEGYLSSLHLKAFTYINLDKV
VLGTSNFKVSASPLLYTLMGKIMQDVKHPVDGKSLYRDSNWISKVEKLS
FDNAAYPFLAYSGIPAVSFCFCEDADYPYLGTRLDTYEALTQKVPQLNQ
MVRTAAEVAGQLIIKLTHDVELNLDYEMYNSKLLSFMKDLNQFKTDIRD
MGLSLQWLYSARGDYFRATSRLTTDFHNAEKTNRFVMREINDRIMKVEY
HFLSPYVSPRESPFRHIFWGSGSHTLSALVENLKLRQKNITAFNETLFR
NQLALATWTIQGVANALSGDIWNIDNEF

In some embodiments, an anti-transferrin receptor 1 antibody binds to an amino acid segment of the receptor as follows: FVKIQVKDSAQNSVIIVDKNGRLVYLVENPGGYVAYSKAATVTGKLVHANFGTKKDFE DLYTPVNGSIVIVRAGKITFAEKVANAESLNAIGVLIYMDQTKFPIVNAELSFFGHAHLG TGDPYTPGFPSFNHTQFPPSRSSGLPNIPVQTISRAAAEKLFGNMEGDCPSDWKTDSTCR MVTSESKNVKLTVSNVLKE (SEQ ID NO: 109) and does not inhibit the binding interactions between transferrin receptors and transferrin and/or (e.g., and) human hemochromatosis protein (also known as HFE). In some embodiments, the anti-transferrin receptor 1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g., ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g., Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g., a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g., specifically bind, to a transferrin receptor may be internalized into the cell, e.g., through receptor-mediated endocytosis, upon binding to a transferrin receptor.

Provided herein, in some aspects, are humanized antibodies that bind to transferrin receptor with high specificity and affinity. In some embodiments, the humanized anti-TfR1 antibody described herein specifically binds to any extracellular epitope of a transferrin receptor or an epitope that becomes exposed to an antibody. In some embodiments, the humanized anti-TfR1 antibodies provided herein bind specifically to transferrin receptor from human, non-human primates, mouse, rat, etc. In some embodiments, the humanized anti-TfR1 antibodies provided herein bind to human transferrin receptor. In some embodiments, the humanized anti-TfR1 antibody described herein binds to an amino acid segment of a human or non-human primate transferrin receptor, as provided in SEQ ID NOs: 105-108. In some embodiments, the humanized anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, an anti-TfR1 antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a KD of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibodies described herein does not complete with or inhibit transferrin binding to the TfR1. In some embodiments, binding of any one of the anti-TfR1 antibodies described herein does not complete with or inhibit HFE-beta-2-microglobulin binding to the TfR1.

Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY (SEQ ID NO: | DDYMY (SEQ ID NO: 7) | GFNIKDD (SEQ |
| | H1 | 1) | | ID NO: 12) |
| | CDR- | IDPENGDT (SEQ ID NO: | WIDPENGDTEYASKFQD | ENG (SEQ ID NO: |
| | H2 | 2 | (SEQ ID NO: 8) | 13) |
| | CDR- | TLWLRRGLDY (SEQ ID | WLRRGLDY (SEQ ID NO: 9) | LRRGLD (SEQ ID |
| | H3 | NO: 3) | | NO: 14) |
| | CDR- | KSLLHSNGYTY (SEQ ID | RSSKSLLHSNGYTYLF (SEQ | SKSLLHSNGYTY |
| | L1 | NO: 4) | ID NO: 10) | (SEQ ID NO: 15) |
| | CDR- | RMS (SEQ ID NO: 5) | RMSNLAS (SEQ ID NO: 11) | RMS (SEQ ID NO: |
| | L2 | | | 5) |
| | CDR- | MQHLEYPFT (SEQ ID | MQHLEYPFT (SEQ ID NO: 6) | HLEYPF (SEQ ID |
| | L3 | NO: 6) | | NO: 16) |
| | VH | EVQLQQSGAELVRPGASVKLSCTASGFNIKDDYMYWVKQRPEQGLEWIGWI | | |
| | | DPENGDTEYASKFQDKATVTADTSSNTAYLQLSSLTSEDTAVYYCTLWLRRG | | |
| | | LDYWGQGTSVTVSS (SEQ ID NO: 17) | | |
| | VL | DIVMTQAAPSVPVTPGESVSISCRSSKSLLHSNGYTYLFWFLQRPGQSPQLLIY | | |
| | | RMSNLASGVPDRFSGSGSGTAFTLRISRVEAEDVGVYYCMQHLEYPFTFGGG | | |
| | | TKLEIK (SEQ ID NO: 18) | | |
| 3-A4 | CDR- | GFNIKDDY (SEQ ID NO: | DDYMY (SEQ ID NO: 7) | GFNIKDD (SEQ |
| N54T* | H1 | 1) | | ID NO: 12) |
| | CDR- | IDPETGDT (SEQ ID NO: | WIDPETGDTEYASKFQD | ETG (SEQ ID NO: |
| | H2 | 19) | (SEQ ID NO: 20) | 21) |
| | CDR- | TLWLRRGLDY (SEQ ID | WLRRGLDY (SEQ ID NO: 9) | LRRGLD (SEQ ID |
| | H3 | NO: 3) | | NO: 14) |
| | CDR- | KSLLHSNGYTY (SEQ ID | RSSKSLLHSNGYTYLF (SEQ | SKSLLHSNGYTY |
| | L1 | NO: 4) | ID NO: 10) | (SEQ ID NO: 15) |
| | CDR- | RMS (SEQ ID NO: 5) | RMSNLAS (SEQ ID NO: 11) | RMS(SEQ ID NO: |
| | L2 | | | 5) |
| | CDR- | MQHLEYPFT (SEQ ID | MQHLEYPFT (SEQ ID NO: 6) | HLEYPF (SEQ ID |
| | L3 | NO: 6) | | NO: 16) |
| | VH | EVQLQQSGAELVRPGASVKLSCTASGFNIKDDYMYWVKQRPEQGLEWIGWI | | |
| | | DPETGDTEYASKFQDKATVTADTSSNTAYLQLSSLTSEDTAVYYCTLWLRRG | | |
| | | LDYWGQGTSVTVS S (SEQ ID NO: 22) | | |
| | VL | DIVMTQAAPSVPVTPGESVSISCRSSKSLLHSNGYTYLFWFLQRPGQSPQLLIY | | |
| | | RMSNLASGVPDRFSGSGSGTAFTLRISRVEAEDVGVYYCMQHLEYPFTFGGG | | |
| | | TKLEIK (SEQ ID NO: 18) | | |
| 3-A4 | CDR- | GFNIKDDY (SEQ ID NO: | DDYMY (SEQ ID NO: 7) | GFNIKDD (SEQ |
| N54S* | H1 | 1) | | ID NO: 12) |
| | CDR- | IDPESGDT (SEQ ID NO: | WIDPESGDTEYASKFQD | ESG (SEQ ID NO: |
| | H2 | 23) | (SEQ ID NO: 24) | 25) |
| | CDR- | TLWLRRGLDY (SEQ ID | WLRRGLDY (SEQ ID NO: 9) | LRRGLD (SEQ ID |
| | H3 | NO: 3) | | NO: 14) |
| | CDR- | KSLLHSNGYTY (SEQ ID | RSSKSLLHSNGYTYLF (SEQ | SKSLLHSNGYTY |
| | L1 | NO: 4) | ID NO: 10) | (SEQ ID NO: 15) |
| | CDR- | RMS (SEQ ID NO: 5) | RMSNLAS (SEQ ID NO: 11) | RMS (SEQ ID NO: |
| | L2 | | | 5) |
| | CDR- | MQHLEYPFT (SEQ ID | MQHLEYPFT (SEQ ID NO: 6) | HLEYPF (SEQ ID |
| | L3 | NO: 6) | | NO: 16) |
| | VH | EVQLQQSGAELVRPGASVKLSCTASGFNIKDDYMYWVKQRPEQGLEWIGWI | | |
| | | DPESGDTEYASKFQDKATVTADTSSNTAYLQLSSLTSEDTAVYYCTLWLRRG | | |
| | | LDYWGQGTSVTVSS (SEQ ID NO: 26) | | |
| | VL | DIVMTQAAPSVPVTPGESVSISCRSSKSLLHSNGYTYLFWFLQRPGQSPQLLIY | | |
| | | RMSNLASGVPDRFSGSGSGTAFTLRISRVEAEDVGVYYCMQHLEYPFTFGGG | | |
| | | TKLEIK (SEQ ID NO: 18) | | |
| 3-M12 | CDR- | GYSITSGYY (SEQ ID | SGYYWN (SEQ ID NO: 33) | GYSITSGY (SEQ |
| | H1 | NO: 27) | | ID NO: 38) |
| | CDR- | ITFDGAN (SEQ ID NO: | YITFDGANNYNPSLKN (SEQ | FDG (SEQ ID NO: |
| | H2 | 28) | ID NO: 34) | 39) |
| | CDR- | TRSSYDYDVLDY (SEQ | SSYDYDVLDY (SEQ ID NO: | SYDYDVLD (SEQ |
| | H3 | ID NO: 29) | 35) | ID NO: 40) |
| | CDR- | QDISNF (SEQ ID NO: 30) | RASQDISNFLN (SEQ ID NO: | SQDISNF (SEQ ID |
| | L1 | | 36) | NO: 41) |
| | CDR- | YTS (SEQ ID NO: 31) | YTSRLHS (SEQ ID NO: 37) | YTS (SEQ ID NO: |
| | L2 | | | 31) |
| | CDR- | QQGHTLPYT (SEQ ID | QQGHTLPYT (SEQ ID NO: 32) | GHTLPY (SEQ ID |
| | L3 | NO: 32) | | NO: 42) |
| | VH | DVQLQESGPGLVKPSQSLSLTCSVTGYSITSGYYWNWIRQFPGNKLEWMGYIT | | |
| | | FDGANNYNPSLKNRISITRDTSKNQFFLKLTSVTTEDTATYYCTRSSYDYDVL | | |
| | | DYWGQGTTLTVSS (SEQ ID NO: 43) | | |
| | VL | DIQMTQTTSSLSASLGDRVTISCRASQDISNFLNWYQQRPDGTVKLLIYYTSRL | | |
| | | HSGVPSRFSGSGSGTDFSLTVSNLEQEDIATYFCQQGHTLPYTFGGGTKLEIK | | |
| | | (SEQ ID NO: 44) | | |
| 5-H12 | CDR- | GYSFTDYC (SEQ ID NO: | DYCIN (SEQ ID NO: 51) | GYSFTDY (SEQ |
| | H1 | 45) | | ID NO: 56) |
| | CDR- | IYPGSGNT (SEQ ID NO: | WIYPGSGNTRYSERFKG | GSG (SEQ ID NO: |
| | H2 | 46) | (SEQ ID NO: 52) | 57) |
| | CDR- | AREDYYPYHGMDY | EDYYPYHGMDY (SEQ ID | DYYPYHGMD |
| | H3 | (SEQ ID NO: 47) | NO: 53) | (SEQ ID NO: 58) |
| | CDR- | ESVDGYDNSF (SEQ ID | RASESVDGYDNSFMH (SEQ | SESVDGYDNSF |
| | L1 | NO: 48) | ID NO: 54) | (SEQ ID NO: 59) |
| | CDR- | RAS (SEQ ID NO: 49) | RASNLES (SEQ ID NO: 55) | RAS (SEQ ID NO: |
| | L2 | | | 49) |
| | CDR | QQSSEDPWT (SEQ ID | QQSSEDPWT (SEQ ID NO: 50) | SSEDPW (SEQ ID |
| | L3 | NO: 50) | | NO: 60) |
| | VH | QIQLQQSGPELVRPGASVKISCKASGYSFTDYCINWVNQRPGQGLEWIGWIYP | | |
| | | GSGNTRYSERFKGKATLTVDTSSNTAYMQLSSLTSEDSAVYFCAREDYYPYH | | |
| | | GMDYWGQGTSVTVSS (SEQ ID NO: 61) | | |
| | VL | DIVLTQSPTSLAVSLGQRATISCRASESVDGYDNSFMHWYQQKPGQPPKLLIF | | |
| | | RASNLESGIPARFSGSGSRTDFTLTINPVEAADVATYYCQQSSEDPWTFGGGT | | |
| | | KLEIK (SEQ ID NO: 62) | | |
| 5-H12 | CDR- | GYSFTDYY (SEQ ID | DYYIN (SEQ ID NO: 64) | GYSFTDY (SEQ |
| C33Y* | H1 | NO: 63) | | ID NO: 56) |
| | CDR- | IYPGSGNT (SEQ ID NO: | WIYPGSGNTRYSERFKG | GSG (SEQ ID NO: |
| | H2 | 46) | (SEQ ID NO: 52) | 57) |
| | CDR- | AREDYYPYHGMDY | EDYYPYHGMDY (SEQ ID | DYYPYHGMD |
| | H3 | (SEQ ID NO: 47) | NO: 53) | (SEQ ID NO: 58) |
| | CDR- | ESVDGYDNSF (SEQ ID | RASESVDGYDNSFMH (SEQ | SESVDGYDNSF |
| | L1 | NO: 48) | ID NO: 54) | (SEQ ID NO: 59) |
| | CDR- | RAS (SEQ ID NO: 49) | RASNLES (SEQ ID NO: 55) | RAS (SEQ ID NO: |
| | L2 | | | 49) |
| | CDR- | QQSSEDPWT (SEQ ID | QQSSEDPWT (SEQ ID NO: 50) | SSEDPW (SEQ ID |
| | L3 | NO: 50) | | NO: 60) |
| | VH | QIQLQQSGPELVRPGASVKISCKASGYSFTDYYINWVNQRPGQGLEWIGWIYP | | |
| | | GSGNTRYSERFKGKATLTVDTSSNTAYMQLSSLTSEDSAVYFCAREDYYPYH | | |
| | | GMDYWGQGTSVTVSS (SEQ ID NO: 65) | | |
| | VL | DIVLTQSPTSLAVSLGQRATISCRASESVDGYDNSFMHWYQQKPGQPPKLLIF | | |
| | | RASNLESGIPARFSGSGSRTDFTLTINPVEAADVATYYCQQSSEDPWTFGGGT | | |
| | | KLEIK (SEQ ID NO: 62) | | |
| 5-H12 | CDR- | GYSFTDYD (SEQ ID | DYDIN (SEQ ID NO: 67) | GYSFTDY (SEQ |
| C33D* | H1 | NO: 66) | | ID NO: 56) |
| | CDR- | IYPGSGNT (SEQ ID NO: | WIYPGSGNTRYSERFKG | GSG (SEQ ID NO: |
| | H2 | 46) | (SEQ ID NO: 52) | 57) |
| | CDR- | AREDYYPYHGMDY | EDYYPYHGMDY (SEQ ID | DYYPYHGMD |
| | H3 | (SEQ ID NO: 47) | NO: 53) | (SEQ ID NO: 58) |
| | CDR- | ESVDGYDNSF (SEQ ID | RASESVDGYDNSFMH (SEQ | SESVDGYDNSF |
| | L1 | NO: 48) | ID NO: 54) | (SEQ ID NO: 59) |
| | CDR- | RAS (SEQ ID NO: 49) | RASNLES (SEQ ID NO: 55) | RAS (SEQ ID NO: |
| | L2 | | | 49) |
| | CDR- | QQSSEDPWT (SEQ ID | QQSSEDPWT (SEQ ID NO: 50) | SSEDPW (SEQ ID |
| | L3 | NO: 50) | | NO: 60) |

| | |
|---|---|
| VH | QIQLQQSGPELVRPGASVKISCKASGYSFTDYDINWVNQRPGQGLEWIGWIYPG |
| | SGNTRYSERFKGKATLTVDTSSNTAYMQLSSLTSEDSAVYFCAREDYYPYHGM |
| | DYWGQGTSVTVSS (SEQ ID NO: 68) |
| VL | DIVLTQSPTSLAVSLGQRATISCRASESVDGYDNSFMHWYQQKPGQPPKLLIF |
| | RASNLESGIPARFSGSGSRTDFTLTINPVEAADVATYYCQQSSEDPWTFGGGT |
| | KLEIK (SEQ ID NO: 62) |

*mutation positions are according to Kabat numbering of the respective VH sequences containing the mutations

In some embodiments, the anti-TfR1 antibody of the present disclosure is a humanized variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

Humanized antibodies and methods of making them are known, e.g., as described in Almagro et al., Front. Biosci. 13:1619-1633 (2008); Riechmann et al., Nature 332:323-329 (1988); Queen et al., Proc. Nat′l Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan et al., Mol. Immunol. 28:489-498 (1991); Dall′Acqua et al., Methods 36:43-60 (2005); Osbourn et al., Methods 36:61-68 (2005); and Klimka et al., Br. J. Cancer, 83:252-260 (2000), the contents of all of which are incorporated herein by reference. Human framework regions that may be used for humanization are described in e.g., Sims et al. J. Immunol. 151:2296 (1993); Carter et al., Proc. Natl. Acad. Sci. USA, 89:4285 (1992); Presta et al., J. Immunol., 151:2623 (1993); Almagro et al., Front. Biosci. 13:1619-1633 (2008)); Baca et al., J. Biol. Chem. 272:10678-10684 (1997); and Rosok et al., J Biol. Chem. 271:22611-22618 (1996), the contents of all of which are incorporated herein by reference.

In some embodiments, the humanized anti-TfR1 antibody of the present disclosure comprises a humanized VH comprising one or more amino acid variations (e.g., in the VH framework region) as compared with any one of the VHs listed in Table 2, and/or (e.g., and) a humanized VL comprising one or more amino acid variations (e.g., in the VL framework region) as compared with any one of the VLs listed in Table 2.

In some embodiments, the humanized anti-TfR1 antibody of the present disclosure comprises a humanized VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH of any of the anti-TfR1 antibodies listed in Table 2 (e.g., any one of SEQ ID NOs: 17, 22, 26, 43, 61, 65, and 68). Alternatively or in addition (e.g., in addition), the humanized anti-TfR1 antibody of the present disclosure comprises a humanized VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL of any one of the anti-TfR1 antibodies listed in Table 2 (e.g., any one of SEQ ID NOs: 18, 44, and 62).

In some embodiments, the humanized anti-TfR1 antibody of the present disclosure comprises a humanized VH comprising an amino acid sequence that is at least 75% (e.g., 75%, 80%, 85%, 90%, 95%, 98%, or 99%) identical in the framework regions to the VH of any of the anti-TfR1 antibodies listed in Table 2 (e.g., any one of SEQ ID NOs: 17, 22, 26, 43, 61, 65, and 68). Alternatively or in addition (e.g., in addition), In some embodiments, the humanized anti-TfR1 antibody of the present disclosure comprises a humanized VL comprising an amino acid sequence that is at least 75% (e.g., 75%, 80%, 85%, 90%, 95%, 98%, or 99%) identical in the framework regions to the VL of any of the anti-TfR1 antibodies listed in Table 2 (e.g., any one of SEQ ID NOs: 18, 44, and 62).

In some embodiments, the humanized anti-TfR1 antibody of the present disclosure comprises a humanized VH comprising a CDR-H1 having the amino acid sequence of SEQ ID NO: 1 (according to the IMGT definition system), a CDR-H2 having the amino acid sequence of SEQ ID NO: 2, SEQ ID NO: 19, or SEQ ID NO: 23 (according to the IMGT definition system), a CDR-H3 having the amino acid sequence of SEQ ID NO: 3 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 17, SEQ ID NO: 22, or SEQ ID NO: 26. Alternatively or in addition (e.g., in addition), the anti-TfR1 antibody of the present disclosure comprises a humanized VL comprising a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 18.

In some embodiments, the humanized anti-TfR1 antibody of the present disclosure comprises a humanized VH comprising a CDR-H1 having the amino acid sequence of SEQ ID NO: 1 (according to the IMGT definition system), a CDR-H2 having the amino acid sequence of SEQ ID NO: 2, SEQ ID NO: 19, or SEQ ID NO: 23 (according to the IMGT definition system), a CDR-H3 having the amino acid sequence of SEQ ID NO: 3 (according to the IMGT definition system), and is at least 75% (e.g., 75%, 80%, 85%, 90%, 95%, 98%, or 99%) identical in the framework regions to the VH as set forth in SEQ ID NO: 17, SEQ ID NO: 22, or SEQ ID NO: 26. Alternatively or in addition (e.g., in addition), the humanized anti-TfR1 antibody of the present disclosure comprises a humanized VL comprising a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system), and is at least 75% (e.g., 75%, 80%, 85%, 90%, 95%, 98%, or 99%) identical in the framework regions to the VL as set forth in any one of SEQ ID NO: 18.

In some embodiments, the humanized anti-TfR1 antibody of the present disclosure comprises a humanized VH comprising a CDR-H1 having the amino acid sequence of SEQ ID NO: 7 (according to the Kabat definition system), a CDR-H2 having the amino acid sequence of SEQ ID NO: 8, SEQ ID NO: 20, or SEQ ID NO: 24 (according to the Kabat definition system), a CDR-H3 having the amino acid sequence of SEQ ID NO: 9 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 17, SEQ ID NO: 22, or SEQ ID NO: 26. Alternatively or in addition (e.g., in addition), the humanized anti-TfR1 antibody of the present disclosure comprises a humanized VL comprising a CDR-L1 having the amino acid sequence of SEQ ID NO: 10 (according to the Kabat definition system), a CDR-L2 having the amino acid sequence of SEQ ID NO: 11 (according to the Kabat definition system), and a CDR-L3 having the amino acid sequence of SEQ ID NO: 6 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 18.

In some embodiments, the humanized anti-TfR1 antibody of the present disclosure comprises a humanized VH comprising a CDR-H1 having the amino acid sequence of SEQ ID NO: 7 (according to the Kabat definition system), a CDR-H2 having the amino acid sequence of SEQ ID NO: 8, SEQ ID NO: 20, or SEQ ID NO: 24 (according to the Kabat definition system), a CDR-H3 having the amino acid sequence of SEQ ID NO: 9 (according to the Kabat definition system), and is at least 75% (e.g., 75%, 80%, 85%, 90%, 95%, 98%, or 99%) identical in the framework regions to the VH as set forth in SEQ ID NO: 17, SEQ ID NO: 22, or SEQ ID NO: 26. Alternatively or in addition (e.g., in addition), the humanized anti-TfR1 antibody of the present disclosure comprises a humanized VL comprising a CDR-L1 having the amino acid sequence of SEQ ID NO: 10 (according to the Kabat definition system), a CDR-L2 having the amino acid sequence of SEQ ID NO: 11 (according to the Kabat definition system), and a CDR-L3 having the amino acid sequence of SEQ ID NO: 6 (according to the Kabat definition system), and is at least 75% (e.g., 75%, 80%, 85%, 90%, 95%, 98%, or 99%) identical in the framework regions to the VL as set forth in any one of SEQ ID NO: 18.

In some embodiments, the humanized anti-TfR1 antibody of the present disclosure comprises a humanized VH comprising a CDR-H1 having the amino acid sequence of SEQ ID NO: 12 (according to the Chothia definition system), a CDR-H2 having the amino acid sequence of SEQ ID NO: 13, SEQ ID NO: 21, or SEQ ID NO: 25 (according to the Chothia definition system), a CDR-H3 having the amino acid sequence of SEQ ID NO: 14 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 17, SEQ ID NO: 22 or SEQ ID NO: 26. Alternatively or in addition (e.g., in addition), the humanized anti-TfR1 antibody of the present disclosure comprises a humanized VL comprising a CDR-L1 having the amino acid sequence of SEQ ID NO: 15 (according to the Chothia definition system), a CDR-L2 having the amino acid sequence of SEQ ID NO: 5 (according to the Chothia definition system), and a CDR-L3 having the amino acid sequence of SEQ ID NO: 16 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 18.

In some embodiments, the humanized anti-TfR1 antibody of the present disclosure comprises a humanized VH comprising a CDR-H1 having the amino acid sequence of SEQ ID NO: 12 (according to the Chothia definition system), a CDR-H2 having the amino acid sequence of SEQ ID NO: 13, SEQ ID NO: 21, or SEQ ID NO: 25 (according to the Chothia definition system), a CDR-H3 having the amino acid sequence of SEQ ID NO: 14 (according to the Chothia definition system), and is at least 75% (e.g., 75%, 80%, 85%, 90%, 95%, 98%, or 99%) identical in the framework regions to the VH as set forth in SEQ ID NO: SEQ ID NO: 17, SEQ ID NO: 22 or SEQ ID NO: 26. Alternatively or in addition (e.g., in addition), the anti-TfR1 antibody of the present disclosure comprises a humanized VL comprising a CDR-L1 having the amino acid sequence of SEQ ID NO: 15 (according to the Chothia definition system), a CDR-L2 having the amino acid sequence of SEQ ID NO: 5 (according to the Chothia definition system), and a CDR-L3 having the amino acid sequence of SEQ ID NO: 16 (according to the Chothia definition system), and is at least 75% (e.g., 75%, 80%, 85%, 90%, 95%, 98%, or 99%) identical in the framework regions to the VL as set forth in any one of SEQ ID NO: 18.

In some embodiments, the humanized anti-TfR1 antibody of the present disclosure comprises a humanized VH comprising a CDR-H1 having the amino acid sequence of SEQ ID NO: 27 (according to the IMGT definition system), a CDR-H2 having the amino acid sequence of SEQ ID NO: 28 (according to the IMGT definition system), a CDR-H3 having the amino acid sequence of SEQ ID NO: 29 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 43. Alternatively or in addition (e.g., in addition), the humanized anti-TfR1 antibody of the present disclosure comprises a humanized VL comprising a CDR-L1 having the amino acid sequence of SEQ ID NO: 30 (according to the IMGT definition system), a CDR-L2 having the amino acid sequence of SEQ ID NO: 31 (according to the IMGT definition system), and a CDR-L3 having the amino acid sequence of SEQ ID NO: 32 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 44.

In some embodiments, the humanized anti-TfR1 antibody of the present disclosure comprises a humanized VH comprising a CDR-H1 having the amino acid sequence of SEQ ID NO: 27 (according to the IMGT definition system), a CDR-H2 having the amino acid sequence of SEQ ID NO: 28 (according to the IMGT definition system), a CDR-H3 having the amino acid sequence of SEQ ID NO: 29 (according to the IMGT definition system), and is at least 75% (e.g., 75%, 80%, 85%, 90%, 95%, 98%, or 99%) identical in the framework regions to the VH as set forth in SEQ ID NO: 43. Alternatively or in addition (e.g., in addition), the humanized anti-TfR1 antibody of the present disclosure comprises a humanized VL comprising a CDR-L1 having the amino acid sequence of SEQ ID NO: 30 (according to the IMGT definition system), a CDR-L2 having the amino acid sequence of SEQ ID NO: 31 (according to the IMGT definition system), and a CDR-L3 having the amino acid sequence of SEQ ID NO: 32 (according to the IMGT definition system), and is at least 75% (e.g., 75%, 80%, 85%, 90%, 95%, 98%, or 99%) identical in the framework regions to the VL as set forth in SEQ ID NO: 44.

In some embodiments, the humanized anti-TfR1 antibody of the present disclosure comprises a humanized VH comprising a CDR-H1 having the amino acid sequence of SEQ ID NO: 33 (according to the Kabat definition system), a CDR-H2 having the amino acid sequence of SEQ ID NO: 34 (according to the Kabat definition system), a CDR-H3 having the amino acid sequence of SEQ ID NO: 35 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 43. Alternatively or in addition (e.g., in addition), the humanized anti-TfR1 antibody of the present disclosure comprises a humanized VL comprising a CDR-L1 having the amino acid sequence of SEQ ID NO: 36 (according to the Kabat definition system), a CDR-L2 having the amino acid sequence of SEQ ID NO: 37 (according to the Kabat definition system), and a CDR-L3 having the amino acid sequence of SEQ ID NO: 32 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 44.

In some embodiments, the humanized anti-TfR1 antibody of the present disclosure comprises a humanized VH comprising a CDR-H1 having the amino acid sequence of SEQ ID NO: 33 (according to the Kabat definition system), a CDR-H2 having the amino acid sequence of SEQ ID NO: 34 (according to the Kabat definition system), a CDR-H3 having the amino acid sequence of SEQ ID NO: 35 (according to the Kabat definition system), and is at least 75% (e.g., 75%, 80%, 85%, 90%, 95%, 98%, or 99%) identical in the framework regions to the VH as set forth in SEQ ID NO: 43. Alternatively or in addition (e.g., in addition), the humanized anti-TfR1 antibody of the present disclosure comprises a humanized VL comprising a CDR-L1 having the amino acid sequence of SEQ ID NO: 36 (according to the Kabat definition system), a CDR-L2 having the amino acid sequence of SEQ ID NO: 37 (according to the Kabat definition system), and a CDR-L3 having the amino acid sequence of SEQ ID NO: 32 (according to the Kabat definition system), and is at least 75% (e.g., 75%, 80%, 85%, 90%, 95%, 98%, or 99%) identical in the framework regions to the VL as set forth in SEQ ID NO: 44.

In some embodiments, the humanized anti-TfR1 antibody of the present disclosure comprises a humanized VH comprising a CDR-H1 having the amino acid sequence of SEQ ID NO: 38 (according to the Chothia definition system), a CDR-H2 having the amino acid sequence of SEQ ID NO: 39 (according to the Chothia definition system), a CDR-H3 having the amino acid sequence of SEQ ID NO: 40 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 43. Alternatively or in addition (e.g., in addition), the humanized anti-TfR1 antibody of the present disclosure comprises a humanized VL comprising a CDR-L1 having the amino acid sequence of SEQ ID NO: 41 (according to the Chothia definition system), a CDR-L2 having the amino acid sequence of SEQ ID NO: 31 (according to the Chothia definition system), and a CDR-L3 having the amino acid sequence of SEQ ID NO: 42 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 44.

In some embodiments, the humanized anti-TfR1 antibody of the present disclosure comprises a humanized VH comprising a CDR-H1 having the amino acid sequence of SEQ ID NO: 38 (according to the Chothia definition system), a CDR-H2 having the amino acid sequence of SEQ ID NO: 39 (according to the Chothia definition system), a CDR-H3 having the amino acid sequence of SEQ ID NO: 40 (according to the Chothia definition system), and is at least 75% (e.g., 75%, 80%, 85%, 90%, 95%, 98%, or 99%) identical in the framework regions to the VH as set forth in SEQ ID NO: 43. Alternatively or in addition (e.g., in addition), the humanized anti-TfR1 antibody of the present disclosure comprises a humanized VL comprising a CDR-L1 having the amino acid sequence of SEQ ID NO: 41 (according to the Chothia definition system), a CDR-L2 having the amino acid sequence of SEQ ID NO: 31 (according to the Chothia definition system), and a CDR-L3 having the amino acid sequence of SEQ ID NO: 42 (according to the Chothia definition system), and is at least 75% (e.g., 75%, 80%, 85%, 90%, 95%, 98%, or 99%) identical in the framework regions to the VL as set forth in SEQ ID NO: 44.

In some embodiments, the humanized anti-TfR1 antibody of the present disclosure comprises a humanized VH comprising a CDR-H1 having the amino acid sequence of SEQ ID NO: 45, SEQ ID NO: 63, or SEQ ID NO: 66 (according to the IMGT definition system), a CDR-H2 having the amino acid sequence of SEQ ID NO: 46 (according to the IMGT definition system), a CDR-H3 having the amino acid sequence of SEQ ID NO: 47 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 61, SEQ ID NO: 65, or SEQ ID NO: 68. Alternatively or in addition (e.g., in addition), the humanized anti-TfR1 antibody of the present disclosure comprises a humanized VL comprising a CDR-L1 having the amino acid sequence of SEQ ID NO: 48 (according to the IMGT definition system), a CDR-L2 having the amino acid sequence of SEQ ID NO: 49 (according to the IMGT definition system), and a CDR-L3 having the amino acid sequence of SEQ ID NO: 50 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 62.

In some embodiments, the humanized anti-TfR1 antibody of the present disclosure comprises a humanized VH comprising a CDR-H1 having the amino acid sequence of SEQ ID NO: 45, SEQ ID NO: 63, or SEQ ID NO: 66 (according to the IMGT definition system), a CDR-H2 having the amino acid sequence of SEQ ID NO: 46 (according to the IMGT definition system), a CDR-H3 having the amino acid sequence of SEQ ID NO: 47 (according to the IMGT definition system), and is at least 75% (e.g., 75%, 80%, 85%, 90%, 95%, 98%, or 99%) identical in the framework regions to the VH as set forth in SEQ ID NO: 61, SEQ ID NO: 65, SEQ ID NO: 68. Alternatively or in addition (e.g., in addition), the humanized anti-TfR1 antibody of the present disclosure comprises a humanized VL comprising a CDR-L1 having the amino acid sequence of SEQ ID NO: 48 (according to the IMGT definition system), a CDR-L2 having the amino acid sequence of SEQ ID NO: 49 (according to the IMGT definition system), and a CDR-L3 having the amino acid sequence of SEQ ID NO: 50 (according to the IMGT definition system), and is at least 75% (e.g., 75%, 80%, 85%, 90%, 95%, 98%, or 99%) identical in the framework regions to the VL as set forth in SEQ ID NO: 62.

In some embodiments, the humanized anti-TfR1 antibody of the present disclosure comprises a humanized VH comprising a CDR-H1 having the amino acid sequence of SEQ ID NO: 51, SEQ ID NO: 64, or SEQ ID NO: 67 (according to the Kabat definition system), a CDR-H2 having the amino acid sequence of SEQ ID NO: 52 (according to the Kabat definition system), a CDR-H3 having the amino acid sequence of SEQ ID NO: 53 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 61, SEQ ID NO: 65, SEQ ID NO: 68. Alternatively or in addition (e.g., in addition), the humanized anti-TfR1 antibody of the present disclosure comprises a humanized VL comprising a CDR-L1 having the amino acid sequence of SEQ ID NO: 54 (according to the Kabat definition system), a CDR-L2 having the amino acid sequence of SEQ ID NO: 55 (according to the Kabat definition system), and a CDR-L3 having the amino acid sequence of SEQ ID NO: 50 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 62.

In some embodiments, the humanized anti-TfR1 antibody of the present disclosure comprises a humanized VH comprising a CDR-H1 having the amino acid sequence of SEQ ID NO: 51, SEQ ID NO: 64, or SEQ ID NO: 67 (according to the Kabat definition system), a CDR-H2 having the amino acid sequence of SEQ ID NO: 52 (according to the Kabat definition system), a CDR-H3 having the amino acid sequence of SEQ ID NO: 53 (according to the Kabat definition system), and is at least 75% (e.g., 75%, 80%, 85%, 90%, 95%, 98%, or 99%) identical in the framework regions to the VH as set forth in SEQ ID NO: 61, SEQ ID NO: 65, SEQ ID NO: 68. Alternatively or in addition (e.g., in addition), the humanized anti-TfR1 antibody of the present disclosure comprises a humanized VL comprising a CDR-L1 having the amino acid sequence of SEQ ID NO: 54 (according to the Kabat definition system), a CDR-L2 having the amino acid sequence of SEQ ID NO: 55 (according to the Kabat definition system), and a CDR-L3 having the amino acid sequence of SEQ ID NO: 50 (according to the Kabat definition system), and is at least 75% (e.g., 75%, 80%, 85%, 90%, 95%, 98%, or 99%) identical in the framework regions to the VL as set forth in SEQ ID NO: 62.

In some embodiments, the humanized anti-TfR1 antibody of the present disclosure comprises a humanized VH comprising a CDR-H1 having the amino acid sequence of SEQ ID NO: 56 (according to the Chothia definition system), a CDR-H2 having the amino acid sequence of SEQ ID NO: 57 (according to the Chothia definition system), a CDR-H3 having the amino acid sequence of SEQ ID NO: 58 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 61, SEQ ID NO: 65, SEQ ID NO: 68. Alternatively or in addition (e.g., in addition), the humanized anti-TfR1 antibody of the present disclosure comprises a humanized VL comprising a CDR-L1 having the amino acid sequence of SEQ ID NO: 59 (according to the Chothia definition system), a CDR-L2 having the amino acid sequence of SEQ ID NO: 49 (according to the Chothia definition system), and a CDR-L3 having the amino acid sequence of SEQ ID NO: 60 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 62.

In some embodiments, the humanized anti-TfR1 antibody of the present disclosure comprises a humanized VH comprising a CDR-H1 having the amino acid sequence of SEQ ID NO: 56 (according to the Chothia definition system), a CDR-H2 having the amino acid sequence of SEQ ID NO: 57 (according to the Chothia definition system), a CDR-H3 having the amino acid sequence of SEQ ID NO: 58 (according to the Chothia definition system), and is at least 75% (e.g., 75%, 80%, 85%, 90%, 95%, 98%, or 99%) identical in the framework regions to the VH as set forth in SEQ ID NO: 61, SEQ ID NO: 65, SEQ ID NO: 68. Alternatively or in addition (e.g., in addition), the humanized anti-TfR1 antibody of the present disclosure comprises a humanized VL comprising a CDR-L1 having the amino acid sequence of SEQ ID NO: 59 (according to the Chothia definition system), a CDR-L2 having the amino acid sequence of SEQ ID NO: 49 (according to the Chothia definition system), and a CDR-L3 having the amino acid sequence of SEQ ID NO: 60 (according to the Chothia definition system), and is at least 75% (e.g., 75%, 80%, 85%, 90%, 95%, 98%, or 99%) identical in the framework regions to the VL as set forth in SEQ ID NO: 62.

Examples of amino acid sequences of the humanized anti-TfR1 antibodies described herein are provided in Table 3.

TABLE 3
Variable Regions of Humanized Anti-TfR1 Antibodies

| Antibody | Variable Region Amino Acid Sequence** |
|---|---|
| 3A4 | VH: |
| VH3 (N54T*)/Vκ4 | EVQLVQSGSELKKPGASVKVSCTASGFNIKDDYMYWVRQPPGKGLEWIGWIDP |
| | ETGDTEYASKFQDRVTVTADTSTNTAYMELSSLRSEDTAVYYCTLWLRRGLD |
| | YWGQGTLVTVSS (SEQ ID NO: 69) |
| | VL: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3A4 | VH: |
| VH3 (N54S*)/Vκ4 | EVQLVQSGSELKKPGASVKVSCTASGFNIKDDYMYWVRQPPGKGLEWIGWIDP |
| | ESGDTEYASKFQDRVTVTADTSTNTAYMELSSLRSEDTAVYYCTLWLRRGLD |
| | YWGQGTLVTVSS (SEQ ID NO: 71) |
| | VL: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3A4 | VH: |
| VH3/Vκ4 | EVQLVQSGSELKKPGASVKVSCTASGFNIKDDYMYWVRQPPGKGLEWIGWIDP |
| | ENGDTEYASKFQDRVTVTADTSTNTAYMELSSLRSEDTAVYYCTLWLRRGLD |
| | YWGQGTLVTVSS (SEQ ID NO: 72) |
| | VL: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3M12 | VH: |
| VH3/Vκ2 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | VH: |
| VH3/Vκ3 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 3M12 | VH: |
| VH4/Vκ2 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | VH: |
| VH4/Vκ3 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 5H12 | VH: |
| VH5 (C33Y*)/Vκ3 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 77) |
| | VL: |
| | DIVLTQSPDSLAVSLGERATINCRASESVDGYDNSFMHWYQQKPGQPPKLLIFR |
| | ASNLESGVPDRFSGSGSRTDFTLTISSLQAEDVAVYYCQQSSEDPWTFGQGTKL |
| | EIK (SEQ ID NO: 78) |
| 5H12 | VH: |
| VH5 (C33D*)/Vκ4 | QVQLVQSGAEVKKPGASVKVSCKASGYSFTDYDINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 79) |
| | VL: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKL |
| | EIK (SEQ ID NO: 80) |
| 5H12 | VH: |
| VH5 (C33Y*)/Vκ4 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 77) |
| | VL: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKL |
| | EIK (SEQ ID NO: 80) |

*mutation positions are according to Kabat numbering of the respective VH sequences containing the mutations
**CDRs according to the Kabat numbering system are bolded

In some embodiments, the humanized anti-TfR1 antibody of the present disclosure comprises a humanized VH comprising the CDR-H1, CDR-H2, and CDR-H3 of any one of the anti-TfR1 antibodies provided in Table 2 and comprises one or more (e.g., 1, 2, 3, 4, 5, 6, 7, 8, 9, 10 or more) amino acid variations in the framework regions as compared with the respective humanized VH provided in Table 3. Alternatively or in addition (e.g., in addition), the humanized anti-TfR1 antibody of the present disclosure comprises a humanized VL comprising the CDR-L1, CDR-L2, and CDR-L3 of any one of the anti-TfR1 antibodies provided in Table 2 and comprises one or more (e.g., 1, 2, 3, 4, 5, 6, 7, 8, 9, 10 or more) amino acid variations in the framework regions as compared with the respective humanized VL provided in Table 3.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 69, and/or (e.g., and) a humanized VL comprising an amino acid sequence that is at least 80% identical (e.g., 80%, 85%, 90%, 95%, 98%, or 99%) to SEQ ID NO: 70. In some embodiments, the humanized anti-TfR1 antibody of the present disclosure comprises a humanized VH comprising the amino acid sequence of SEQ ID NO: 69 and a humanized VL comprising the amino acid sequence of SEQ ID NO: 70.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 71, and/or (e.g., and) a humanized VL comprising an amino acid sequence that is at least 80% identical (e.g., 80%, 85%, 90%, 95%, 98%, or 99%) to SEQ ID NO: 70. In some embodiments, the humanized anti-TfR1 antibody of the present disclosure comprises a humanized VH comprising the amino acid sequence of SEQ ID NO: 71 and a humanized VL comprising the amino acid sequence of SEQ ID NO: 70.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 72, and/or (e.g., and) a humanized VL comprising an amino acid sequence that is at least 80% identical (e.g., 80%, 85%, 90%, 95%, 98%, or 99%) to SEQ ID NO: 70. In some embodiments, the humanized anti-TfR1 antibody of the present disclosure comprises a humanized VH comprising the amino acid sequence of SEQ ID NO: 72 and a humanized VL comprising the amino acid sequence of SEQ ID NO: 70.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 73, and/or (e.g., and) a humanized VL comprising an amino acid sequence that is at least 80% identical (e.g., 80%, 85%, 90%, 95%, 98%, or 99%) to SEQ ID NO: 74. In some embodiments, the humanized anti-TfR1 antibody of the present disclosure comprises a humanized VH comprising the amino acid sequence of SEQ ID NO: 73 and a humanized VL comprising the amino acid sequence of SEQ ID NO: 74.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 73, and/or (e.g., and) a humanized VL comprising an amino acid sequence that is at least 80% identical (e.g., 80%, 85%, 90%, 95%, 98%, or 99%) to SEQ ID NO: 75. In some embodiments, the humanized anti-TfR1 antibody of the present disclosure comprises a humanized VH comprising the amino acid sequence of SEQ ID NO: 73 and a humanized VL comprising the amino acid sequence of SEQ ID NO: 75.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 76, and/or (e.g., and) a humanized VL comprising an amino acid sequence that is at least 80% identical (e.g., 80%, 85%, 90%, 95%, 98%, or 99%) to SEQ ID NO: 74. In some embodiments, the humanized anti-TfR1 antibody of the present disclosure comprises a humanized VH comprising the amino acid sequence of SEQ ID NO: 76 and a humanized VL comprising the amino acid sequence of SEQ ID NO: 74.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 76, and/or (e.g., and) a humanized VL comprising an amino acid sequence that is at least 80% identical (e.g., 80%, 85%, 90%, 95%, 98%, or 99%) to SEQ ID NO: 75. In some embodiments, the humanized anti-TfR1 antibody of the present disclosure comprises a humanized VH comprising the amino acid sequence of SEQ ID NO: 76 and a humanized VL comprising the amino acid sequence of SEQ ID NO: 75.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 77, and/or (e.g., and) a humanized VL comprising an amino acid sequence that is at least 80% identical (e.g., 80%, 85%, 90%, 95%, 98%, or 99%) to SEQ ID NO: 78. In some embodiments, the humanized anti-TfR1 antibody of the present disclosure comprises a humanized VH comprising the amino acid sequence of SEQ ID NO: 77 and a humanized VL comprising the amino acid sequence of SEQ ID NO: 78.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 79, and/or (e.g., and) a humanized VL comprising an amino acid sequence that is at least 80% identical (e.g., 80%, 85%, 90%, 95%, 98%, or 99%) to SEQ ID NO: 80. In some embodiments, the humanized anti-TfR1 antibody of the present disclosure comprises a humanized VH comprising the amino acid sequence of SEQ ID NO: 79 and a humanized VL comprising the amino acid sequence of SEQ ID NO: 80.

In some embodiments, the humanized anti-TfR1 antibody of the present disclosure comprises a humanized VH comprising an amino acid sequence that is at least 80% (e.g., 80%, 85%, 90%, 95%, 98%, or 99%) identical to SEQ ID NO: 77, and/or (e.g., and) a humanized VL comprising an amino acid sequence that is at least 80% identical (e.g., 80%, 85%, 90%, 95%, 98%, or 99%) to SEQ ID NO: 80. In some embodiments, the humanized anti-TfR1 antibody of the present disclosure comprises a humanized VH comprising the amino acid sequence of SEQ ID NO: 77 and a humanized VL comprising the amino acid sequence of SEQ ID NO: 80.

In some embodiments, the humanized anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other antibody heavy and light chain constant regions are well known in the art, e.g., those provided in the IMGT database (www.imgt.org) or at www.vbase2.org/vbstat.php., both of which are incorporated by reference herein.

In some embodiments, the humanized anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the humanized anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the humanized anti-TfR1 antibody described herein comprises a heavy chain comprising any one of the VH as listed in Table 3 or any variants thereof and a heavy chain constant region as set forth in SEQ ID NO: 81. In some embodiments, the humanized anti-TfR1 antibody described herein comprises heavy chain comprising any one of the VH as listed in Table 3 or any variants thereof and a heavy chain constant region as set forth in SEQ ID NO: 82.

In some embodiments, the humanized anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the humanized anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the humanized anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of examples of humanized anti-TfR1 IgGs

| Antibody | IgG Heavy Chain/Light Chain Sequences** |
|---|---|
| 3A4 | Heavy Chain (with wild type human IgG1 constant region) |
| VH3 (N54T*)/Vκ4 | EVQLVQSGSELKKPGASVKVSCTASGFNIKDDYMYWVRQPPGKGLEWIGW |
| | IDPETGDTEYASKFQDRVTVTADTSTNTAYMELSSLRSEDTAVYYCTLWL |
| | RRGLDYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 84) |
| | Light Chain (with kappa light chain constant region) |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTE |
| | GGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 85) |
| 3A4 | Heavy Chain (with wild type human IgG1 constant region) |
| VH3 (N54S*)/Vκ4 | EVQLVQSGSELKKPGASVKVSCTASGFNIKDDYMYWVRQPPGKGLEWIGW |
| | IDPESGDTEYASKFQDRVTVTADTSTNTAYMELSSLRSEDTAVYYCTLWL |
| | RRGLDYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 86) |
| | Light Chain (with kappa light chain constant region) |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTE |
| | GGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 85) |
| 3A4 | Heavy Chain (with wild type human IgG1 constant region) |
| VH3/Vκ4 | EVQLVQSGSELKKPGASVKVSCTASGFNIKDDYMYWVRQPPGKGLEWIGW |
| | IDPENGDTEYASKFQDRVTVTADTSTNTAYMELSSLRSEDTAVYYCTLWL |
| | RRGLDYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 87) |
| | Light Chain (with kappa light chain constant region) |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTF |
| | GGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 85) |
| 3M12 | Heavy Chain (with wild type human IgG1 constant region) |
| VH3/Vκ2 | QVQLQESGPGLVKPSQTLSLTCSVTGYSITSGYYWNWIRQPPGKGLEWMGY |
| | ITFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELT |
| | KNQVSLTCLVKGFYPSDIA VEWESNGQPENNYKTTPPVLDSDGSFFLYSKLT |
| | VDKSRWQQGNVFSCSVMHEALHNHYTQKSLSLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYFCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 89) |
| 3M12 | Heavy Chain (with wild type human IgG1 constant region) |
| VH3/Vκ3 | QVQLQESGPGLVKPSQTLSLTCSVTGYSITSGYYWNWIRQPPGKGLEWMGY |
| | ITFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLT |
| | VDKSRWQQGNVFSCSVMHEALHNHYTQKSLSLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYYCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 90) |
| 3M12 | Heavy Chain (with wild type human IgG1 constant region) |
| VH4/Vκ2 | QVQLQESGPGLVKPSQTLSLTCTVTGYSITSGYYWNWIRQPPGKGLEWIGYI |
| | TFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLT |
| | VDKSRWQQGNVFSCSVMHEALHNHYTQKSLSLSPGK (SEQ ID NO: 91) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYFCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 89) |
| 3M12 | Heavy Chain (with wild type human IgG1 constant region) |
| VH4/Vκ3 | QVQLQESGPGLVKPSQTLSLTCTVTGYSITSGYYWNWIRQPPGKGLEWIGYI |
| | TFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLT |
| | VDKSRWQQGNVFSCSVMHEALHNHYTQKSLSLSPGK (SEQ ID NO: 91) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYYCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 90) |
| 5H12 | Heavy Chain (with wild type human IgG1 constant region) |
| VH5 (C33Y*)/Vκ3 | QVQLVQSGAEVKKPGASVKVSCKASGYSFTDYYINWVRQAPGQGLEWMG |
| | WIYPGSGNTRYSERFKGRVTITRDTSASTAYMELSSLRSEDTAVYYCARED |
| | YYPYHGMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK (SEQ ID NO: |
| | 92) |
| | Light Chain (with kappa light chain constant region) |
| | DIVLTQSPDSLAVSLGERATINCRASESVDGYDNSFMHWYQQKPGQPPKLL |
| | IFRASNLESGVPDRFSGSGSRTDFTLTISSLQAEDVAVYYCQQSSEDPWTFG |
| | QGTKLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLS |
| | SPVTKSFNRGEC (SEQ ID NO: 93) |
| 5H12 | Heavy Chain (with wild type human IgG1 constant region) |
| VH5 (C33D*)/Vκ4 | QVQLVQSGAEVKKPGASVKVSCKASGYSFTDYDINWVRQAPGQGLEWMG |
| | WIYPGSGNTRYSERFKGRVTITRDTSASTAYMELSSLRSEDTAVYYCARED |
| | YYPYHGMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK (SEQ ID NO: |
| | 94) |
| | Light Chain (with kappa light chain constant region) |
| | DIVMTQSPDSLAVSLGERATINCRASESVDGYDNSFMHWYQQKPGQPPKL |
| | LIFRASNLESGVPDRFSGSGSGTDFTLTISSLQAEDVAVYYCQQSSEDPWTF |
| | GQGTKLEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 95) |
| 5H12 | Heavy Chain (with wild type human IgG1 constant region) |
| VH5 (C33Y*)/Vκ4 | QVQLVQSGAEVKKPGASVKVSCKASGYSFTDYYINWVRQAPGQGLEWMG |
| | WIYPGSGNTRYSERFKGRVTITRDTSASTAYMELSSLRSEDTAVYYCARED |
| | YYPYHGMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK (SEQ ID NO: |
| | 92) |
| | Light Chain (with kappa light chain constant region) |
| | DIVMTQSPDSLAVSLGERATINCRASESVDGYDNSFMHWYQQKPGQPPKL |
| | LIFRASNLESGVPDRFSGSGSGTDFTLTISSLQAEDVAVYYCQQSSEDPWTF |
| | GQGTKLEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 95) |

*mutation positions are according to Kabat numbering of the respective VH sequences containing the mutations
**CDRs according to the Kabat numbering system are bolded; VH/VL sequences underlined

In some embodiments, the humanized anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9,8, 7, 6,5,4, 3, 2, or 1 amino acid variation) as compared with the heavy chain as set forth in any one of SEQ ID NOs: 84, 86, 87, 88, 91, 92, and 94. Alternatively or in addition (e.g., in addition), the humanized anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, and 95.

In some embodiments, the humanized anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, and 94. Alternatively or in addition (e.g., in addition), the humanized anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, and 95. In some embodiments, the anti-TfR1 antibody described herein comprises a heavy chain comprising the amino acid sequence of any one of SEQ ID NOs: 84, 86, 87, 88, 91, 92, and 94. Alternatively or in addition (e.g., in addition), the anti-TfR1 antibody described herein comprises a light chain comprising the amino acid sequence of any one of SEQ ID NOs: 85, 89, 90, 93, and 95.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 84, and/or (e.g., and) a light chain comprising an amino acid sequence that is at least 80% identical (e.g., 80%, 85%, 90%, 95%, 98%, or 99%) to SEQ ID NO: 85. In some embodiments, the humanized anti-TfR1 antibody of the present disclosure comprises a heavy chain comprising the amino acid sequence of SEQ ID NO: 84 and a light chain comprising the amino acid sequence of SEQ ID NO: 85.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 86, and/or (e.g., and) a light chain comprising an amino acid sequence that is at least 80% identical (e.g., 80%, 85%, 90%, 95%, 98%, or 99%) to SEQ ID NO: 85. In some embodiments, the humanized anti-TfR1 antibody of the present disclosure comprises a heavy chain comprising the amino acid sequence of SEQ ID NO: 86 and a light chain comprising the amino acid sequence of SEQ ID NO: 85.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 87, and/or (e.g., and) a light chain comprising an amino acid sequence that is at least 80% identical (e.g., 80%, 85%, 90%, 95%, 98%, or 99%) to SEQ ID NO: 85. In some embodiments, the humanized anti-TfR1 antibody of the present disclosure comprises a heavy chain comprising the amino acid sequence of SEQ ID NO: 87 and a light chain comprising the amino acid sequence of SEQ ID NO: 85.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 88, and/or (e.g., and) a light chain comprising an amino acid sequence that is at least 80% identical (e.g., 80%, 85%, 90%, 95%, 98%, or 99%) to SEQ ID NO: 89. In some embodiments, the humanized anti-TfR1 antibody of the present disclosure comprises a heavy chain comprising the amino acid sequence of SEQ ID NO: 88 and a light chain comprising the amino acid sequence of SEQ ID NO: 89.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 88, and/or (e.g., and) a light chain comprising an amino acid sequence that is at least 80% identical (e.g., 80%, 85%, 90%, 95%, 98%, or 99%) to SEQ ID NO: 90. In some embodiments, the humanized anti-TfR1 antibody of the present disclosure comprises a heavy chain comprising the amino acid sequence of SEQ ID NO: 88 and a light chain comprising the amino acid sequence of SEQ ID NO: 90.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 91, and/or (e.g., and) a light chain comprising an amino acid sequence that is at least 80% identical (e.g., 80%, 85%, 90%, 95%, 98%, or 99%) to SEQ ID NO: 89. In some embodiments, the humanized anti-TfR1 antibody of the present disclosure comprises a heavy chain comprising the amino acid sequence of SEQ ID NO: 91 and a light chain comprising the amino acid sequence of SEQ ID NO: 89.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 91, and/or (e.g., and) a light chain comprising an amino acid sequence that is at least 80% identical (e.g., 80%, 85%, 90%, 95%, 98%, or 99%) to SEQ ID NO: 90. In some embodiments, the humanized anti-TfR1 antibody of the present disclosure comprises a heavy chain comprising the amino acid sequence of SEQ ID NO: 91 and a light chain comprising the amino acid sequence of SEQ ID NO: 90.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 92, and/or (e.g., and) a light chain comprising an amino acid sequence that is at least 80% identical (e.g., 80%, 85%, 90%, 95%, 98%, or 99%) to SEQ ID NO: 93. In some embodiments, the humanized anti-TfR1 antibody of the present disclosure comprises a heavy chain comprising the amino acid sequence of SEQ ID NO: 92 and a light chain comprising the amino acid sequence of SEQ ID NO: 93.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 94, and/or (e.g., and) a light chain comprising an amino acid sequence that is at least 80% identical (e.g., 80%, 85%, 90%, 95%, 98%, or 99%) to SEQ ID NO: 95. In some embodiments, the humanized anti-TfR1 antibody of the present disclosure comprises a heavy chain comprising the amino acid sequence of SEQ ID NO: 94 and a light chain comprising the amino acid sequence of SEQ ID NO: 95.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 92, and/or (e.g., and) a light chain comprising an amino acid sequence that is at least 80% identical (e.g., 80%, 85%, 90%, 95%, 98%, or 99%) to SEQ ID NO: 95. In some embodiments, the humanized anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody is a Fab fragment, Fab′ fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full-length IgG using an enzyme such as papain). For example, F(ab′)₂ fragments can be produced by pepsin or papain digestion of an antibody molecule, and Fab′ fragments that can be generated by reducing the disulfide bridges of F(ab′)₂ fragments. In some embodiments, a heavy chain constant region in a Fab fragment of the anti-TfR1 antibody described herein comprises the amino acid sequence of:

(SEQ ID NO: 96)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHT

In some embodiments, the humanized anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the humanized anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the humanized anti-TfR1 antibody described herein comprises a heavy chain comprising any one of the VH as listed in Table 3 or any variants thereof and a heavy chain constant region as set forth in SEQ ID NO: 96.

In some embodiments, the humanized anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the humanized anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the humanized anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of examples of humanized anti-TfR1 Fabs

| Antibody | Fab Heavy Chain/Light Chain Sequences** |
|---|---|
| 3A4 | Heavy Chain (with partial human IgG1 constant region) |
| VH3 (N54T*)/Vκ4 | EVQLVQSGSELKKPGASVKVSCTASGFNIKDDYMYWVRQPPGKGLEWIGW |
| | IDPETGDTEYASKFQDRVTVTADTSTNTAYMELSSLRSEDTAVYYCTLWL |
| | RRGLDYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHT (SEQ ID NO: 97) |
| | Light Chain (with kappa light chain constant region) |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTF |
| | GGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 85) |
| 3A4 | Heavy Chain (with partial human IgG1 constant region) |
| VH3 (N54S*)/Vκ4 | EVQLVQSGSELKKPGASVKVSCTASGFNIKDDYMYWVRQPPGKGLEWIGW |
| | IDPESGDTEYASKFQDRVTVTADTSTNTAYMELSSLRSEDTAVYYCTLWL |
| | RRGLDYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHT (SEQ ID NO: 98) |
| | Light Chain (with kappa light chain constant region) |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTF |
| | GGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 85) |
| 3A4 | Heavy Chain (with partial human IgG1 constant region) |
| VH3/VK4 | EVQLVQSGSELKKPGASVKVSCTASGFNIKDDYMYWVRQPPGKGLEWIGW |
| | IDPENGDTEYASKFQDRVTVTADTSTNTAYMELSSLRSEDTAVYYCTLWL |
| | RRGLDYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHT (SEQ ID NO: 99) |
| | Light Chain (with kappa light chain constant region) |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTF |
| | GGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 85) |
| 3M12 | Heavy Chain (with partial human IgG1 constant region) |
| VH3/Vκ2 | QVQLQESGPGLVKPSQTLSLTCSVTGYSITSGYYWNWIRQPPGKGLEWMGY |
| | ITFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHT (SEQ ID NO: 100) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYFCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 89) |
| 3M12 | Heavy Chain (with partial human IgG1 constant region) |
| VH3/Vκ3 | QVQLQESGPGLVKPSQTLSLTCSVTGYSITSGYYWNWIRQPPGKGLEWMGY |
| | ITFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHT (SEQ ID NO: 100) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYYCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 90) |
| 3M12 | Heavy Chain (with partial human IgG1 constant region) |
| VH4/Vκ2 | QVQLQESGPGLVKPSQTLSLTCTVTGYSITSGYYWNWIRQPPGKGLEWIGYI |
| | TFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHT (SEQ ID NO: 101) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYFCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 89) |
| 3M12 | Heavy Chain (with partial human IgG1 constant region) |
| VH4/Vκ3 | QVQLQESGPGLVKPSQTLSLTCTVTGYSITSGYYWNWIRQPPGKGLEWIGYI |
| | TFDGANNYNPSLKNRVSISRDTSKNQFSLKLSSVTAEDTATYYCTRSSYDY |
| | DVLDYWGQGTTVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHT (SEQ ID NO: 101) |
| | Light Chain (with kappa light chain constant region) |
| | DIQMTQSPSSLSASVGDRVTITCRASQDISNFLNWYQQKPGQPVKLLIYYTS |
| | RLHSGVPSRFSGSGSGTDFTLTISSLQPEDFATYYCQQGHTLPYTFGQGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 90) |
| 5H12 | Heavy Chain (with partial human IgG1 constant region) |
| VH5 (C33Y*)/Vκ3 | QVQLVQSGAEVKKPGASVKVSCKASGYSFTDYYINWVRQAPGQGLEWMG |
| | WIYPGSGNTRYSERFKGRVTITRDTSASTAYMELSSLRSEDTAVYYCARED |
| | YYPYHGMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYIC |
| | NVNHKPSNTKVDKKVEPKSCDKTHT (SEQ ID NO: 102) |
| | Light Chain (with kappa light chain constant region) |
| | DIVLTQSPDSLAVSLGERATINCRASESVDGYDNSFMHWYQQKPGQPPKLL |
| | IFRASNLESGVPDRFSGSGSRTDFTLTISSLQAEDVAVYYCQQSSEDPWTFG |
| | QGTKLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLS |
| | SPVTKSFNRGEC (SEQ ID NO: 93) |
| 5H12 | Heavy Chain (with partial human IgG1 constant region) |
| VH5 (C33D*)/Vκ4 | QVQLVQSGAEVKKPGASVKVSCKASGYSFTDYDINWVRQAPGQGLEWMG |
| | WIYPGSGNTRYSERFKGRVTITRDTSASTAYMELSSLRSEDTAVYYCARED |
| | YYPYHGMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYIC |
| | NVNHKPSNTKVDKKVEPKSCDKTHT (SEQ ID NO: 103) |
| | Light Chain (with kappa light chain constant region) |
| | DIVMTQSPDSLAVSLGERATINCRASESVDGYDNSFMHWYQQKPGQPPKL |
| | LIFRASNLESGVPDRFSGSGSGTDFTLTISSLQAEDVAVYYCQQSSEDPWTF |
| | GQGTKLEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 95) |
| 5H12 | Heavy Chain (with partial human IgG1 constant region) |
| VH5 (C33Y*)/Vκ4 | QVQLVQSGAEVKKPGASVKVSCKASGYSFTDYYINWVRQAPGQGLEWMG |
| | WIYPGSGNTRYSERFKGRVTITRDTSASTAYMELSSLRSEDTAVYYCARED |
| | YYPYHGMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYIC |
| | NVNHKPSNTKVDKKVEPKSCDKTHT (SEQ ID NO: 102) |
| | Light Chain (with kappa light chain constant region) |
| | DIVMTQSPDSLAVSLGERATINCRASESVDGYDNSFMHWYQQKPGQPPKL |
| | LIFRASNLESGVPDRFSGSGSGTDFTLTISSLQAEDVAVYYCQQSSEDPWTF |
| | GQGTKLEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGL |
| | SSPVTKSFNRGEC (SEQ ID NO: 95) |

*mutation positions are according to Kabat numbering of the respective VH sequences containing the mutations
**CDRs according to the Kabat numbering system are bolded; VH/VL sequences underlined

In some embodiments, the humanized anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9,8, 7, 6,5, 4, 3,2, or 1 amino acid variation) as compared with the heavy chain as set forth in any one of SEQ ID NOs: 97-103. Alternatively or in addition (e.g., in addition), the humanized anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, and 95.

In some embodiments, the humanized anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103. Alternatively or in addition (e.g., in addition), the humanized anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, and 95. In some embodiments, the anti-TfR1 antibody described herein comprises a heavy chain comprising the amino acid sequence of any one of SEQ ID NOs: 97-103. Alternatively or in addition (e.g., in addition), the anti-TfR1 antibody described herein comprises a light chain comprising the amino acid sequence of any one of SEQ ID NOs: 85, 89, 90, 93, and 95.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 97, and/or (e.g., and) a light chain comprising an amino acid sequence that is at least 80% identical (e.g., 80%, 85%, 90%, 95%, 98%, or 99%) to SEQ ID NO: 85. In some embodiments, the humanized anti-TfR1 antibody of the present disclosure comprises a heavy chain comprising the amino acid sequence of SEQ ID NO: 97 and a light chain comprising the amino acid sequence of SEQ ID NO: 85.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 98, and/or (e.g., and) a light chain comprising an amino acid sequence that is at least 80% identical (e.g., 80%, 85%, 90%, 95%, 98%, or 99%) to SEQ ID NO: 85. In some embodiments, the humanized anti-TfR1 antibody of the present disclosure comprises a heavy chain comprising the amino acid sequence of SEQ ID NO: 98 and a light chain comprising the amino acid sequence of SEQ ID NO: 85.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 99, and/or (e.g., and) a light chain comprising an amino acid sequence that is at least 80% identical (e.g., 80%, 85%, 90%, 95%, 98%, or 99%) to SEQ ID NO: 85. In some embodiments, the humanized anti-TfR1 antibody of the present disclosure comprises a heavy chain comprising the amino acid sequence of SEQ ID NO: 99 and a light chain comprising the amino acid sequence of SEQ ID NO: 85.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 100, and/or (e.g., and) a light chain comprising an amino acid sequence that is at least 80% identical (e.g., 80%, 85%, 90%, 95%, 98%, or 99%) to SEQ ID NO: 89. In some embodiments, the humanized anti-TfR1 antibody of the present disclosure comprises a heavy chain comprising the amino acid sequence of SEQ ID NO: 100 and a light chain comprising the amino acid sequence of SEQ ID NO: 89.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 100, and/or (e.g., and) a light chain comprising an amino acid sequence that is at least 80% identical (e.g., 80%, 85%, 90%, 95%, 98%, or 99%) to SEQ ID NO: 90. In some embodiments, the humanized anti-TfR1 antibody of the present disclosure comprises a heavy chain comprising the amino acid sequence of SEQ ID NO: 100 and a light chain comprising the amino acid sequence of SEQ ID NO: 90.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 101, and/or (e.g., and) a light chain comprising an amino acid sequence that is at least 80% identical (e.g., 80%, 85%, 90%, 95%, 98%, or 99%) to SEQ ID NO: 89. In some embodiments, the humanized anti-TfR1 antibody of the present disclosure comprises a heavy chain comprising the amino acid sequence of SEQ ID NO: 101 and a light chain comprising the amino acid sequence of SEQ ID NO: 89.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 101, and/or (e.g., and) a light chain comprising an amino acid sequence that is at least 80% identical (e.g., 80%, 85%, 90%, 95%, 98%, or 99%) to SEQ ID NO: 90. In some embodiments, the humanized anti-TfR1 antibody of the present disclosure comprises a heavy chain comprising the amino acid sequence of SEQ ID NO: 101 and a light chain comprising the amino acid sequence of SEQ ID NO: 90.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 102, and/or (e.g., and) a light chain comprising an amino acid sequence that is at least 80% identical (e.g., 80%, 85%, 90%, 95%, 98%, or 99%) to SEQ ID NO: 93. In some embodiments, the humanized anti-TfR1 antibody of the present disclosure comprises a heavy chain comprising the amino acid sequence of SEQ ID NO: 102 and a light chain comprising the amino acid sequence of SEQ ID NO: 93.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 103, and/or (e.g., and) a light chain comprising an amino acid sequence that is at least 80% identical (e.g., 80%, 85%, 90%, 95%, 98%, or 99%) to SEQ ID NO: 95. In some embodiments, the humanized anti-TfR1 antibody of the present disclosure comprises a heavy chain comprising the amino acid sequence of SEQ ID NO: 103 and a light chain comprising the amino acid sequence of SEQ ID NO: 95.

In some embodiments, the humanized anti-TfR1 antibody of the present disclosure comprises a heavy chain comprising an amino acid sequence that is at least 80% (e.g., 80%, 85%, 90%, 95%, 98%, or 99%) identical to SEQ ID NO: 102, and/or (e.g., and) a light chain comprising an amino acid sequence that is at least 80% identical (e.g., 80%, 85%, 90%, 95%, 98%, or 99%) to SEQ ID NO: 95. In some embodiments, the humanized anti-TfR1 antibody of the present disclosure comprises a heavy chain comprising the amino acid sequence of SEQ ID NO: 102 and a light chain comprising the amino acid sequence of SEQ ID NO: 95.

In some embodiments, the humanized anti-TfR1 receptor antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, humanized the anti-TfR1 antibody described herein is a scFv. In some embodiments, the humanized anti-TfR1 antibody described herein is a scFv-Fab (e.g., scFv fused to a portion of a constant region). In some embodiments, the anti-TfR1 receptor antibody described herein is a scFv fused to a constant region (e.g., human IgG1 constant region as set forth in SEQ ID NO: 81 or SEQ ID NO: 82, or a portion thereof such as the Fc portion) at either the N-terminus of C-terminus.

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall′Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g., a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the Fab heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

### Other Known Anti-Transferrin Receptor Antibodies

Any other appropriate anti-transferrin receptor 1 antibodies known in the art may be used as the muscle-targeting agent in the complexes disclosed herein. Examples of known anti-transferrin receptor 1 antibodies, including associated references and binding epitopes, are listed in Table 6. In some embodiments, the anti-transferrin receptor 1 antibody comprises the complementarity determining regions (CDR-H1, CDR-H2, CDR-H3, CDR-L1, CDR-L2, and CDR-L3) of any of the anti-transferrin receptor 1 antibodies provided herein, e.g., anti-transferrin receptor 1 antibodies listed in Table 6.

TABLE 6
List of anti-transferrin receptor 1 antibody clones, including
associated references and binding epitope information.

| Antibody | | |
|---|---|---|
| Clone Name | Reference(s) | Epitope/Notes |
| OKT9 | U.S. Pat. No. 4,364,934, filed Dec. 4, 1979, | Apical domain |
| | entitled “MONOCLONAL ANTIBODY | of TfR (residues |
| | TO A HUMAN EARLY THYMOCYTE | 305-366 of human |
| | ANTIGEN AND METHODS FOR | TfR sequence |
| | PREPARING SAME” | XM_052730.3 |
| | Schneider C. et al. “Structural features of | (SEQ ID NO: |
| | the cell surface receptor for transferrin that | 404), available |
| | is recognized by the monoclonal antibody | in GenBank) |
| | OKT9.” J Biol Chem. 1982, 257: 14, 8516- | |
| | 8522. | |
| (From JCR) | WO 2015/098989, filed Dec. 24, 2014, | Apical domain |
| Clone M11 | “Novel anti-Transferrin receptor antibody | (residues 230- |
| Clone M23 | that passes through blood-brain barrier” | 244 and 326-347 |
| Clone M27 | U.S. Pat. No. 9,994,641, filed Dec. 24, 2014, | of TfR) and |
| Clone B84 | “Novel anti-Transferrin receptor antibody | protease-like |
| | that passes through blood-brain barrier” | domain (residues |
| | | 461-473) |
| (From Genentech) | WO 2016/081643, filed May 26, 2016, | Apical domain |
| 7A4, 8A2, 15D2, | entitled “ANTI-TRANSFERRIN | and non-apical |
| 10D11, 7B10, | RECEPTOR ANTIBODIES AND | regions |
| 15G11, 16G5, | METHODS OF USE” | |
| 13C3, 16G4, | U.S. Pat. No. 9,708,406, filed May 20, | |
| 16F6, 7G7, | 2014, “Anti-transferrin receptor | |
| 4C2, 1B12, | antibodiesand methods of use” | |
| and 13D4 | | |
| (From Armagen) | Lee et al. “Targeting Rat Anti-Mouse | |
| 8D3 | Transferrin Receptor Monoclonal Antibodies | |
| | through Blood-Brain Barrier in Mouse” 2000, | |
| | J Pharmacol. Exp. Ther., 292: 1048-1052. | |
| | U.S. Pat. App. 2010/077498, filed Sep. 11, | |
| | 2008, entitled “COMPOSITIONS AND | |
| | METHODS FOR BLOOD-BRAIN | |
| | BARRIER DELIVERY IN THE MOUSE” | |
| OX26 | Haobam, B. et al. 2014. Rab17-mediated | |
| | recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of the | |
| | human transferrin receptor in plant cells: | |
| | effects of tyrphostin A23 and brefeldin A. | |
| | Plant J 48: 757-70 (2006). | |
| 1A1B2, 66IG10, | Commercially available anti-transferrin | Novus Biologicals |
| MEM-189, JF0956, | receptor antibodies. | 8100 Southpark |
| 29806, 1A1B2, | | Way, A-8 Littleton |
| TFRC/1818, | | CO 80120 |
| 1E6, 66Ig10, | | |
| TFRC/1059, | | |
| Q1/71, 23D10, | | |
| 13E4, TFRC/1149, | | |
| ER-MP21, | | |
| YTA74.4, BU54, | | |
| 2B6, RI7 217 | | |
| (From INSERM) | U.S. Pat. App. 2011/0311544A1, filed | Does not compete |
| | Jun. 15, 2005, entitled “ANTI-CD71 | with OKT9 |
| BA120g | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed Jun. 7, 2004, | “LUCA31 epitope” |
| | entitled “TRANSFERRIN RECEPTOR | |
| | ANTIBODIES” | |
| (Salk Institute) | Trowbridge, I.S. et al. “Anti-transferrin | |
| B3/25 | receptor monoclonal antibody and toxin- | |
| T58/30 | antibody conjugates affect growth of human | |
| | tumour cells.” | |
| | Nature, 1981, volume 294, pages 171-173 | |
| R17 217.1.3, | Commercially available anti-transferrin | BioXcell |
| 5E9C11, OKT9 | receptor antibodies. | 10 Technology |
| (BE0023 clone) | | Dr., Suite 2B |
| | | West Lebanon, |
| | | NH 03784-1671 |
| BK19.9, B3/25, | Gatter, K.C. et al. “Transferrin receptors | USA |
| T56/14 and | in human tissues: their distribution and | |
| T58/1 | possible clinical relevance.” J Clin Pathol. | |
| | 1983 May; 36(5): 539-45. | |

In some embodiments, anti-TfR1 antibodies of the present disclosure include one or more of the CDR-H (e.g., CDR-H1, CDR-H2, and CDR-H3) amino acid sequences from any one of the anti-TfR1 antibodies selected from Table 6. In some embodiments, anti-TfR1 antibodies include the CDR-H1, CDR-H2, and CDR-H3 as provided for any one of the anti-TfR1 antibodies selected from Table 6. In some embodiments, anti-TfR1 antibodies include the CDR-L1, CDR-L2, and CDR-L3 as provided for any one of the anti-TfR1antibodies selected from Table 6. In some embodiments, anti-TfR1antibodies include the CDR-H1, CDR-H2, CDR-H3, CDR-L1, CDR-L2, and CDR-L3 as provided for any one of the anti-TfR1 antibodies selected from Table 6. The disclosure also includes any nucleic acid sequence that encodes a molecule comprising a CDR-H1, CDR-H2, CDR-H3, CDR-L1, CDR-L2, or CDR-L3 as provided for any one of the anti-TfR1 antibodies selected from Table 6. In some embodiments, antibody heavy and light chain CDR3 domains may play a particularly important role in the binding specificity/affinity of an antibody for an antigen. Accordingly, anti-TfR1 antibodies of the disclosure may include at least the heavy and/or (e.g., and) light chain CDR3s of any one of the anti-TfR1 antibodies selected from Table 6.

In some examples, any of the anti-TfR1antibodies of the disclosure have one or more CDR (e.g., CDR-H or CDR-L) sequences substantially similar to any of the CDR-H1, CDR-H2, CDR-H3, CDR-L1, CDR-L2, and/or (e.g., and) CDR-L3 sequences from one of the anti-TfR1 antibodies selected from Table 6. In some embodiments, the position of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or (e.g., and)C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or (e.g., and) CDR-H3 described herein may be one, two, three, four, five or more amino acids shorter than one or more of the CDRs described herein (e.g., CDRs from any of the anti-transferrin receptor 1 antibodies selected from Table 6) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or (e.g., and) CDR-H3 described herein may be one, two, three, four, five or more amino acids longer than one or more of the CDRs described herein (e.g., CDRs from any of the anti-transferrin receptor 1 antibodies selected from Table 6) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRs from any of the anti-transferrin receptor 1 antibodies selected from Table 6) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRs from any of the anti-transferrin receptor 1 antibodies selected from Table 6) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRs from any of the anti-transferrin receptor 1 antibodies selected from Table 6) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRs from any of the anti-transferrin receptor 1 antibodies selected from Table 6) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-transferrin receptor 1 antibodies of the disclosure have one or more CDR (e.g., CDR-H or CDR-L) sequences substantially similar to any one of the anti-transferrin receptor 1 antibodies selected from Table 6. For example, the antibodies may include one or more CDR sequence(s) from any of the anti-transferrin receptor 1 antibodies selected from Table 6 containing up to 5, 4, 3, 2, or 1 amino acid residue variations as compared to the corresponding CDR region in any one of the CDRs provided herein (e.g., CDRs from any of the anti-transferrin receptor 1 antibodies selected from Table 6) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide transferrin receptor antibodies that comprise one or more of the heavy chain variable (VH) and/or (e.g., and) light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-transferrin receptor 1 antibodies selected from Table 6. In some embodiments, any of the VL domains provided herein include one or more of the CDR-L sequences (e.g., CDR-L1, CDR-L2, and CDR-L3) provided herein, for example, any of the CDR-L sequences provided in any one of the anti-transferrin receptor 1 antibodies selected from Table 6.

In some embodiments, anti-TfR1antibodies of the disclosure include any antibody that includes a heavy chain variable domain and/or (e.g., and) a light chain variable domain of any anti-transferrin receptor 1 antibody, such as any one of the anti-TfR1 antibodies selected from Table 6. In some embodiments, anti-TfR1 antibodies of the disclosure include any antibody that includes the heavy chain variable and light chain variable pairs of any anti-transferrin receptor 1 antibody, such as any one of the anti-TfR1antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1antibody, such as any one of the anti-TfR1antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1antibodies selected from Table 6.

In some embodiments, an anti-transferrin receptor 1 antibody, which specifically binds to transferrin receptor (e.g., human transferrin receptor), comprises a light chain variable VL domain comprising any of the CDR-L domains (CDR-L1, CDR-L2, and CDR-L3), or CDR-L domain variants provided herein, of any of the anti-transferrin receptor 1 antibodies selected from Table 6. In some embodiments, an anti-transferrin receptor 1 antibody, which specifically binds to transferrin receptor (e.g., human transferrin receptor), comprises a light chain variable VL domain comprising the CDR-L1, the CDR-L2, and the CDR-L3 of any anti-transferrin receptor 1 antibody, such as any one of the anti-transferrin receptor 1 antibodies selected from Table 6. In some embodiments, the anti-transferrin receptor 1 antibody comprises a light chain variable (VL) region sequence comprising one, two, three or four of the framework regions of the light chain variable region sequence of any anti-transferrin receptor 1 antibody, such as any one of the anti-transferrin receptor 1 antibodies selected from Table 6. In some embodiments, the anti-transferrin receptor 1 antibody comprises one, two, three or four of the framework regions of a light chain variable region sequence which is at least 75%, 80%, 85%, 90%, 95%, or 100% identical to one, two, three or four of the framework regions of the light chain variable region sequence of any anti-transferrin receptor 1 antibody, such as any one of the anti-transferrin receptor 1 antibodies selected from Table 6. In some embodiments, the light chain variable framework region that is derived from said amino acid sequence consists of said amino acid sequence but for the presence of up to 10 amino acid substitutions, deletions, and/or (e.g., and) insertions, preferably up to 10 amino acid substitutions. In some embodiments, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region.

In some embodiments, an anti-transferrin receptor 1 antibody that specifically binds to transferrin receptor comprises the CDR-L1, the CDR-L2, and the CDR-L3 of any anti-transferrin receptor 1 antibody, such as any one of the anti-transferrin receptor 1 antibodies selected from Table 6. In some embodiments, the antibody further comprises one, two, three or all four VL framework regions derived from the VL of a human or primate antibody. The primate or human light chain framework region of the antibody selected for use with the light chain CDR sequences described herein, can have, for example, at least 70% (e.g., at least 75%, 80%, 85%, 90%, 95%, 98%, or at least 99%) identity with a light chain framework region of a non-human parent antibody. The primate or human antibody selected can have the same or substantially the same number of amino acids in its light chain complementarity determining regions to that of the light chain complementarity determining regions of any of the antibodies provided herein, e.g., any of the anti-transferrin receptor 1 antibodies selected from Table 6. In some embodiments, the primate or human light chain framework region amino acid residues are from a natural primate or human antibody light chain framework region having at least 75% identity, at least 80% identity, at least 85% identity, at least 90% identity, at least 95% identity, at least 98% identity, at least 99% (or more) identity with the light chain framework regions of any anti-transferrin receptor 1 antibody, such as any one of the anti-transferrin receptor 1 antibodies selected from Table 6. In some embodiments, an anti-transferrin receptor 1 antibody further comprises one, two, three or all four VL framework regions derived from a human light chain variable kappa subfamily. In some embodiments, an anti-transferrin receptor 1 antibody further comprises one, two, three or all four VL framework regions derived from a human light chain variable lambda subfamily.

In some embodiments, any of the anti-transferrin receptor 1 antibodies provided herein comprise a light chain variable domain that further comprises a light chain constant region. In some embodiments, the light chain constant region is a kappa, or a lambda light chain constant region. In some embodiments, the kappa or lambda light chain constant region is from a mammal, e.g., from a human, monkey, rat, or mouse. In some embodiments, the light chain constant region is a human kappa light chain constant region. In some embodiments, the light chain constant region is a human lambda light chain constant region. It should be appreciated that any of the light chain constant regions provided herein may be variants of any of the light chain constant regions provided herein. In some embodiments, the light chain constant region comprises an amino acid sequence that is at least 75%, 80%, 85%, 90%, 95%, 98%, or 99% identical to any of the light chain constant regions of any anti-transferrin receptor 1 antibody, such as any one of the anti-transferrin receptor 1 antibodies selected from Table 6.

In some embodiments, the anti-transferrin receptor 1 antibody is any anti-transferrin receptor 1 antibody, such as any one of the anti-transferrin receptor 1 antibodies selected from Table 6.

In some embodiments, an anti-transferrin receptor 1 antibody comprises a VL domain comprising the amino acid sequence of any anti-transferrin receptor 1 antibody, such as any one of the anti-transferrin receptor 1 antibodies selected from Table 6, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In some embodiments, an anti-transferrin receptor 1 antibody comprises any of the VL domains, or VL domain variants, and any of the VH domains, or VH domain variants, wherein the VL and VH domains, or variants thereof, are from the same antibody clone, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the muscle-targeting agent is a transferrin receptor antibody (e.g., the antibody and variants thereof as described in International Application Publication WO 2016/081643, incorporated herein by reference).

The heavy chain and light chain CDRs of the antibody according to different definition systems are provided in Table 7. The different definition systems, e.g., the Kabat definition, the Chothia definition, and/or (e.g., and) the contact definition have been described. See, e.g., (e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, A1-lazikani et al (1997) J. Molec. Biol. 273:927-948; and Almagro, J. Mol. Recognit. 17:132-143 (2004). See also hgmp.mrc.ac.uk and bioinf.org.uk/abs).

An example of a transferrin receptor antibody that may be used in accordance with the present disclosure is described in International Application Publication WO 2016/081643, incorporated herein by reference. The CDR amino acid sequences of this antibody are provided in Table 7.

TABLE 7
Heavy chain and light chain CDRs of a mouse transferrin receptor antibody

| Sequence Type | Kabat | Chothia | Contact |
|---|---|---|---|
| CDR-H1 | SYWMH (SEQ ID | GYTFTSY (SEQ ID | TSYWMH (SEQ ID NO: |
| | NO: 110) | NO: 116) | 118) |
| CDR-H2 | EINPTNGRTNYIE | NPTNGR (SEQ ID | WIGEINPTNGRTN (SEQ |
| | KFKS (SEQ ID NO: | NO: 117) | ID NO: 119) |
| | 111) | | |
| CDR-H3 | GTRAYHY (SEQ | GTRAYHY (SEQ ID NO: | ARGTRA (SEQ ID NO: |
| | ID NO: 112) | 112) | 120) |
| CDR-L1 | RASDNLYSNLA | RASDNLYSNLA (SEQ ID | YSNLAWY (SEQ ID NO: |
| | (SEQ ID NO: 113) | NO: 113) | 121) |
| CDR-L2 | DATNLAD (SEQ | DATNLAD (SEQ ID NO: | LLVYDATNLA (SEQ ID |
| | ID NO: 114) | 114) | NO: 122) |
| CDR-L3 | QHFWGTPLT | QHFWGTPLT (SEQ ID | QHFWGTPL (SEQ ID |
| | (SEQ ID NO: 115) | NO: 115) | NO: 123) |

The heavy chain variable domain (VH) and light chain variable domain sequences are also provided:

VH
(SEQ ID NO: 124)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSS
VL
(SEQ ID NO: 125)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELK

In some embodiments, the anti-TfR1antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1, CDR-H2, and CDR-H3 as shown in Table 7. “Collectively” means that the total number of amino acid variations in all of the three heavy chain CDRs is within the defined range. Alternatively or in addition (e.g., in addition), the anti-TfR1antibody of the present disclosure may comprise a CDR-L1, a CDR-L2, and a CDR-L3, which collectively contains no more than 5 amino acid variations (e.g., no more than 5, 4, 3, 2 or 1 amino acid variation) as compared with the CDR-L1, CDR-L2, and CDR-L3 as shown in Table 7.

In some embodiments, the anti-TfR1antibody of the present disclosure comprises a CDR-H1, a CDR-H2, and a CDR-H3, at least one of which contains no more than 3 amino acid variations (e.g., no more than 3, 2, or 1 amino acid variation) as compared with the counterpart heavy chain CDR as shown in Table 7. Alternatively or in addition (e.g., in addition), the anti-TfR1antibody of the present disclosure may comprise CDR-L1, a CDR-L2, and a CDR-L3, at least one of which contains no more than 3 amino acid variations (e.g., no more than 3, 2, or 1 amino acid variation) as compared with the counterpart light chain CDR as shown in Table 7.

In some embodiments, the anti-TfR1antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1antibody of the present disclosure comprises a CDR-L3 containing one amino acid variation as compared with the CDR-L3 as shown in Table 7. In some embodiments, the anti-TfR1antibody of the present disclosure comprises a CDR-L3 of QHFAGTPLT (SEQ ID NO: 126) (according to the Kabat and Chothia definition system) or QHFAGTPL (SEQ ID NO: 127) (according to the Contact definition system). In some embodiments, the anti-TfR1antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) (according to the Kabat and Chothia definition system) or QHFAGTPL (SEQ ID NO: 127) (according to the Contact definition system).

In some embodiments, the anti-TfR1of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1antibody of the present disclosure comprises a VH comprising the amino acid sequence of SEQ ID NO: 124. Alternatively or in addition (e.g., in addition), the anti-TfR1antibody of the present disclosure comprises a VL comprising the amino acid sequence of SEQ ID NO: 125.

In some embodiments, the anti-TfR1antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 124. Alternatively or in addition (e.g., in addition), the transferrin receptor antibody of the present disclosure comprises a VL comprising an amino acid sequence that is at least 80% (e.g., 80%, 85%, 90%, 95%, or 98%) identical to the VL as set forth in SEQ ID NO: 125.

In some embodiments, the transferrin receptor antibody of the present disclosure is a humanized antibody (e.g., a humanized variant of an antibody). In some embodiments, the transferrin receptor antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 7, and comprises a humanized heavy chain variable region and/or (e.g., and) a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 7) into the IGKV1-NL1*01 and IGHV1-3*01 human variable domains. In some embodiments, the transferrin receptor antibody of the present disclosure is a humanized variant comprising one or more amino acid substitutions at positions 9, 13, 17, 18, 40, 45, and 70 as compared with the VL as set forth in SEQ ID NO: 125, and/or (e.g., and) one or more amino acid substitutions at positions 1, 5, 7, 11, 12, 20, 38, 40, 44, 66, 75, 81, 83, 87, and 108 as compared with the VH as set forth in SEQ ID NO: 124. In some embodiments, the transferrin receptor antibody of the present disclosure is a humanized variant comprising amino acid substitutions at all of positions 9, 13, 17, 18, 40, 45, and 70 as compared with the VL as set forth in SEQ ID NO: 125, and/or (e.g., and) amino acid substitutions at all of positions 1, 5, 7, 11, 12, 20, 38, 40, 44, 66, 75, 81, 83, 87, and 108 as compared with the VH as set forth in SEQ ID NO: 124.

In some embodiments, the transferrin receptor antibody of the present disclosure is a humanized antibody and contains the residues at positions 43 and 48 of the VL as set forth in SEQ ID NO: 125. Alternatively or in addition (e.g., in addition), the transferrin receptor antibody of the present disclosure is a humanized antibody and contains the residues at positions 48, 67, 69, 71, and 73 of the VH as set forth in SEQ ID NO: 124.

The VH and VL amino acid sequences of an example humanized antibody that may be used in accordance with the present disclosure are provided:

Humanized VH
(SEQ ID NO: 128)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSS
Humanized VL
(SEQ ID NO: 129)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIK

In some embodiments, the transferrin receptor antibody of the present disclosure comprises a VH comprising the amino acid sequence of SEQ ID NO: 128. Alternatively or in addition (e.g., in addition), the transferrin receptor antibody of the present disclosure comprise a VL comprising the amino acid sequence of SEQ ID NO: 129.

In some embodiments, the transferrin receptor antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 128. Alternatively or in addition (e.g., in addition), the transferrin receptor antibody of the present disclosure comprises a VL comprising an amino acid sequence that is at least 80% (e.g., 80%, 85%, 90%, 95%, or 98%) identical to the VL as set forth in SEQ ID NO: 129.

In some embodiments, the transferrin receptor antibody of the present disclosure is a humanized variant comprising amino acid substitutions at one or more of positions 43 and 48 as compared with the VL as set forth in SEQ ID NO: 125, and/or (e.g., and) amino acid substitutions at one or more of positions 48, 67, 69, 71, and 73 as compared with the VH as set forth in SEQ ID NO: 124. In some embodiments, the transferrin receptor antibody of the present disclosure is a humanized variant comprising a S43A and/or (e.g., and) a V48L mutation as compared with the VL as set forth in SEQ ID NO: 125, and/or (e.g., and) one or more of A67V, L69I, V71R, and K73T mutations as compared with the VH as set forth in SEQ ID NO: 124.

In some embodiments, the transferrin receptor antibody of the present disclosure is a humanized variant comprising amino acid substitutions at one or more of positions 9, 13, 17, 18, 40, 43, 48, 45, and 70 as compared with the VL as set forth in SEQ ID NO: 125, and/or (e.g., and) amino acid substitutions at one or more of positions 1, 5, 7, 11, 12, 20, 38, 40, 44, 48, 66, 67, 69, 71, 73, 75, 81, 83, 87, and 108 as compared with the VH as set forth in SEQ ID NO: 124.

In some embodiments, the transferrin receptor antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the transferrin receptor antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the heavy chain of any of the transferrin receptor antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 130)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the transferrin receptor antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other antibody heavy and light chain constant regions are well known in the art, e.g., those provided in the IMGT database (www.imgt.org) or at www.vbase2.org/vbstat.php., both of which are incorporated by reference herein.

Examples of heavy chain and light chain amino acid sequences of the transferrin receptor antibodies described are provided below:

Heavy Chain (VH + human IgG1 constant region)
(SEQ ID NO: 132)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light Chain (VL + kappa light chain)
(SEQ ID NO: 133)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Heavy Chain (humanized VH + human IgG1 constant
region)
(SEQ ID NO: 134)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light Chain (humanized VL + kappa light chain)
(SEQ ID NO: 135)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, the transferrin receptor antibody described herein comprises a heavy chain comprising an amino acid sequence that is at least 80% (e.g., 80%, 85%, 90%, 95%, or 98%) identical to SEQ ID NO: 132. Alternatively or in addition (e.g., in addition), the transferrin receptor antibody described herein comprises a light chain comprising an amino acid sequence that is at least 80% (e.g., 80%, 85%, 90%, 95%, or 98%) identical to SEQ ID NO: 133. In some embodiments, the transferrin receptor antibody described herein comprises a heavy chain comprising the amino acid sequence of SEQ ID NO: 132. Alternatively or in addition (e.g., in addition), the transferrin receptor antibody described herein comprises a light chain comprising the amino acid sequence of SEQ ID NO: 133.

In some embodiments, the transferrin receptor antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 132. Alternatively or in addition (e.g., in addition), the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 133.

In some embodiments, the transferrin receptor antibody described herein comprises a heavy chain comprising an amino acid sequence that is at least 80% (e.g., 80%, 85%, 90%, 95%, or 98%) identical to SEQ ID NO: 134. Alternatively or in addition (e.g., in addition), the transferrin receptor antibody described herein comprises a light chain comprising an amino acid sequence that is at least 80% (e.g., 80%, 85%, 90%, 95%, or 98%) identical to SEQ ID NO: 135. In some embodiments, the transferrin receptor antibody described herein comprises a heavy chain comprising the amino acid sequence of SEQ ID NO: 134. Alternatively or in addition (e.g., in addition), the transferrin receptor antibody described herein comprises a light chain comprising the amino acid sequence of SEQ ID NO: 135.

In some embodiments, the transferrin receptor antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain of humanized antibody as set forth in SEQ ID NO: 134. Alternatively or in addition (e.g., in addition), the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain of humanized antibody as set forth in SEQ ID NO: 135.

In some embodiments, the transferrin receptor antibody is an antigen binding fragment (Fab) of an intact antibody (full-length antibody). Antigen binding fragment of an intact antibody (full-length antibody) can be prepared via routine methods. For example, F(ab′)2 fragments can be produced by pepsin digestion of an antibody molecule, and Fab′ fragments that can be generated by reducing the disulfide bridges of F(ab′)2 fragments. Examples of Fab amino acid sequences of the transferrin receptor antibodies described herein are provided below:

Heavy Chain Fab (VH + a portion of human IgG1
constant region)
(SEQ ID NO: 136)
QVQLQQPGAELVKPGASVKLSCKASGYTFTSYWMHWVKQRPGQGLEWI
GEINPTNGRTNYIEKFKSKATLTVDKSSSTAYMQLSSLTSEDSAVYYCA
RGTRAYHYWGQGTSVTVSSASTKGPSVFPLAPSSKSTSGGTAALGCLVK
DYFPEPVTVSWNSGALTSGVHTFPAVLQSSGLYSLSSVVTVPSSSLGTQ
TYICNVNHKPSNTKVDKKVEPKSCDKTHTCP
Heavy Chain Fab (humanized VH + a portion of
human IgG1 constant region)
(SEQ ID NO: 137)
EVQLVQSGAEVKKPGASVKVSCKASGYTFTSYWMHWVRQAPGQRLEW
IGEINPTNGRTNYIEKFKSRATLTVDKSASTAYMELSSLRSEDTAVYYC
ARGTRAYHYWGQGTMVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCP

In some embodiments, the transferrin receptor antibody described herein comprises a heavy chain comprising the amino acid sequence of SEQ ID NO: 136. Alternatively or in addition (e.g., in addition), the transferrin receptor antibody described herein comprises a light chain comprising the amino acid sequence of SEQ ID NO: 133.

In some embodiments, the transferrin receptor antibody described herein comprises a heavy chain comprising the amino acid sequence of SEQ ID NO: 137. Alternatively or in addition (e.g., in addition), the transferrin receptor antibody described herein comprises a light chain comprising the amino acid sequence of SEQ ID NO: 135.

The transferrin receptor antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the transferrin receptor antibody described herein is a scFv. In some embodiments, the transferrin receptor antibody described herein is a scFv-Fab (e.g., scFv fused to a portion of a constant region). In some embodiments, the transferrin receptor antibody described herein is a scFv fused to a constant region (e.g., human IgG1 constant region as set forth in SEQ ID NO: 130).

In some embodiments, any one of the anti-TfR1 antibodies described herein is produced by recombinant DNA technology in Chinese hamster ovary (CHO) cell suspension culture, optionally in CHO-K1 cell (e.g., CHO-K1 cells derived from European Collection of Animal Cell Culture, Cat. No. 85051005) suspension culture.

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

b. Other Muscle-Targeting Antibodies

In some embodiments, the muscle-targeting antibody is an antibody that specifically binds hemojuvelin, caveolin-3, Duchenne muscular dystrophy peptide, or myosin Jib, or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxKl, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor 1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall′Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor 1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like C_{κ} or C_{λ}. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

In some embodiments, the anti-TfR1 antibody of the present disclosure is a humanized antibody comprising human framework regions with the CDRs of a murine antibody listed in Table 1 or Table 2 (e.g., 3A4, 3M12, or 5H12). In some embodiments, the anti-TfR1 antibody of the present disclosure is an IgG1 kappa that comprises human framework regions with the CDRs of a murine antibody listed in Table 1 or Table 2 (e.g., 3A4, 3M12, or 5H12). In some embodiments, the anti-TfR1 antibody of the present disclosure is a Fab fragment of an IgG1 kappa that comprises human framework regions with the CDRs of a murine antibody listed in Table 1 or Table 2 (e.g., 3A4, 3M12, or 5H12).

In some embodiments, any one of the anti-TfR1 antibodies described herein is produced by recombinant DNA technology in Chinese hamster ovary (CHO) cell suspension culture, optionally in CHO-K1 cell (e.g., CHO-K1 cells derived from European Collection of Animal Cell Culture, Cat. No. 85051005) suspension culture.

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. In some embodiments, pyroglutamate formation occurs in a heavy chain sequence. In some embodiments, pyroglutamate formation occurs in a light chain sequence.
ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g., a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 375) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 375). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 376) was identified and this muscle-targeting peptide showed improved binding to C2C12 cells relative to the ASSLNIA (SEQ ID NO: 375) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 377) appeared most frequently. Accordingly, in some embodiments, the muscle-targeting agent comprises the amino acid sequence TARGEHKEEELI (SEQ ID NO: 377).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g., valine, such that the peptide preferentially targets muscle cells (e.g., cardiac muscle cells). In some embodiments, a muscle-targeting peptide has not been previously characterized or disclosed. These peptides may be conceived of, produced, synthesized, and/or derivatized using any of several methodologies, e.g., phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g., Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 378), CSERSMNFC (SEQ ID NO: 379), CPKTRRVPC (SEQ ID NO: 380), WLSEAGPVVTVRALRGTGSW (SEQ ID NO: 381), ASSLNIA (SEQ ID NO: 376), CMQHSMRVC (SEQ ID NO: 382), and DDTRHWG (SEQ ID NO: 383). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally occurring amino acids, e.g., cysteine, alanine, or non-naturally occurring or modified amino acids. Non-naturally occurring amino acids include j-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g., bicyclic (see, e.g., Silvana, M. G. et al. Mol. Therapy, 2018, 26:1, 132-147.).

iii. Muscle-Targeting Receptor Ligands

A muscle-targeting agent may be a ligand, e.g., a ligand that binds to a receptor protein. A muscle-targeting ligand may be a protein, e.g., transferrin, which binds to an internalizing cell surface receptor expressed by a muscle cell (e.g., a cardiac muscle cell). Accordingly, in some embodiments, the muscle-targeting agent is transferrin, or a derivative thereof that binds to a transferrin receptor. A muscle-targeting ligand may alternatively be a small molecule, e.g., a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g., an RNA aptamer, which preferentially targets muscle cells relative to other cell types. In some embodiments, a muscle-targeting aptamer has not been previously characterized or disclosed. These aptamers may be conceived of, produced, synthesized, and/or derivatized using any of several methodologies, e.g., Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g., Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10-15 kDa, about 1-5 Da, about 1-3 kDa, or smaller.

v. Other Muscle-Targeting Agents

One strategy for targeting a muscle cell (e.g., a cardiac muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agents described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KTAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or lack a C-terminal anchoring domain. In some embodiments, hemojuvelin may be annotated under GenBank RefSeq Accession Numbers NM_001316767.1 (SEQ ID NO: 405), NM_145277.4 (SEQ ID NO: 406), NM_202004.3 (SEQ ID NO: 407), NM_213652.3 (SEQ ID NO: 408), or NM_213653.3 (SEQ ID NO: 409). It should be appreciated that a hemojuvelin may be of human, non-human primate, or rodent origin.

### B. Molecular Payloads

Some aspects of the disclosure provide molecular payloads, e.g., for modulating a biological outcome, e.g., the transcription of a DNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of molecular payloads may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a MSTN. In some embodiments, the molecular payload is an oligonucleotide that comprises a strand having a region of complementarity to an INHBA gene (e.g., INHBA DNA or INHBA RNA). In some embodiments, the molecular payload is an oligonucleotide that comprises a strand having a region of complementarity to ACVR1B. In some embodiments, the molecular payload is an oligonucleotide that comprises a strand having a region of complementarity to MLCK1. In some embodiments, the molecular payload is an oligonucleotide that comprises a strand having a region of complementarity to wild-type ACVR1. In some embodiments, the molecular payload is an oligonucleotide that comprises a strand having a region of complementarity to a mutant ACVR1 associated with FOP. In some embodiments, the molecular payload is an oligonucleotide that comprises a strand having a region of complementarity to FBXO32 (e.g., complementarity to NM_001242463.2 (SEQ ID NO: 655), NM_058229.4 (SEQ ID NO: 505), NM_148177.2 (SEQ ID NO: 656), XM_005564029.2 (SEQ ID NO: 657), NM_026346.3 (SEQ ID NO: 506), and/or NM_133521.1 (SEQ ID NO: 658)). In some embodiments, the molecular payload is an oligonucleotide that comprises a strand having a region of complementarity to TRIM63. In some embodiments, the molecular payload is an oligonucleotide that comprises a strand having a region of complementarity to a nucleic acid sequence encoding MEF2D, KLF15, MED1, MED13, or PPP1R3A (e.g., mRNA or DNA). In some embodiments, the molecular payload is an oligonucleotide that comprises a strand having a region of complementarity to the gene encoding MEF2D, KLF15, MED1, MED13, or PPP1R3A. In some embodiments, the molecular payload is an oligonucleotide that comprises a strand having a region of complementarity to a disease allele encoding MEF2D, KLF15, MED1, MED13, or PPP1R3A. In some embodiments, the molecular payload is a DNA decoy, e.g., of a MSTN, INHBA, ACVR1B, MLCK1, ACVR1, FBXO32, TRIM63, MEF2D, KLF15, MED1, MED13, or PPP1R3A nucleic acid. In some embodiments, two or more molecular payloads (e.g., targeting two or more genes) may be linked to a muscle targeting agent. As non-limiting examples, a complex may comprise molecular payloads targeting ACVR1B and MSTN; targeting ACVR1B and INHBA; targeting MSTN and INHBA; or targeting ACVR1B, MSTN and INHBA. Exemplary molecular payloads are described in further detail herein, however, it should be appreciated that the exemplary molecular payloads provided herein are not meant to be limiting.

i. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to promote or increase expression of a gene (e.g., MEF2D, KLF15, MED1, MED13, or PPP1R3A). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format. Oligonucleotides provided herein may be designed to modulate the expression or activity of target genes involved in muscle health, such as muscle growth and maintenance, including MSTN, INHBA and ACVR1B.

In some embodiments, the oligonucleotide is an antisense oligonucleotide (ASO). In some embodiments, the oligonucleotide is a siRNA. In some embodiments, the oligonucleotide is a short hairpin RNA. In some embodiments, the oligonucleotide is a miRNA-based shRNA. In some embodiments, the oligonucleotide is based on a shRNA based on any one of miR-92b-3p, miR-218, miR-18a, miR-1244, and miR-103, as described in Hu et al., Oncotarget. 2017 Nov. 3; 8(54): 92079-92089, and in Chen et al., Oncotarget. 2017 Dec. 19; 8(67): 112152-112165, incorporated herein by reference. In some embodiments, the oligonucleotide is based on a shRNA based on any one of miR-190a-5p, miR-223-3p, and miR-133.

In some embodiments, the oligonucleotide is a CRISPR guide RNA targeting MEF2D, KLF15, MED1, MED13, or PPP1R3A. In some embodiments, the oligonucleotide is a CRISPR guide RNA targeting KLF15 or a promoter region associated with KLF15 (e.g., to increase expression of KLF15).

a. MSTN Oligonucleotides

Examples of oligonucleotides useful for targeting MSTN are provided in Lu-Nguyen, N. et. al. “Functional muscle recovery following dystrophin and myostatin exon splice modulation in aged mdx mice” Human Molecular Genetics, Vol. 28, 18, 3091-3100 (2019); Liu, C. M. et. al. “Myostatin antisense RNA-mediated muscle growth in normal and cancer cachexia mice” Gene Therapy, Vol. 15, 155-160 (2008); Kang, J. K., “Antisense-induced myostatin exon skipping leads to muscle hypertrophy in mice following octa-guanidine morpholino oligomer treatment” Mol Ther. 2011 January; 19(1):159-64.; Kemaladewi, D. U. et. al. “Dual exon skipping in myostatin and dystrophin for Duchenne muscular dystrophy” BMC Med Genomics. 2011 Apr. 20; 4:36.; Tripathi, A. K. et. al. “Short hairpin RNA-induced myostatin gene silencing in caprine myoblast cells in vitro” Appl Biochem Biotechnol. 2013 January; 169(2):688-94.; Lu-Nguyen, N. et. al., “Systemic Antisense Therapeutics for Dystrophin and Myostatin Exon Splice Modulation Improve Muscle Pathology of Adult mdx Mice” Mol. Ther. Nucleic Acids. 2017 Mar. 17; 6:15-28.; U.S. Patent Application Publication 20050124566A1, published on Jun. 5, 2005, entitled “RNA interference mediated inhibition of myostatin gene expression using short interfering nucleic acid (siNA)”; U.S. Pat. No. 10,004,814, issued Jun. 26, 2018, entitled “Systemic delivery of myostatin short interfering nucleic acids (siNA) conjugated to a lipophilic moiety”; U.S. Patent Application Publication 20110166082A1, published on Jul. 7, 2011, entitled “Antisense composition and method for treating muscle atrophy”; U.S. Pat. No. 7,887,793, issued Feb. 15, 2011, entitled “Treatment of Duchenne muscular dystrophy with myoblasts expressing dystrophin and treated to block myostatin signaling”; and U.S. Patent Application Publication 20180355358A1, published on Dec. 13, 2018, entitled “Antisense-induced exon exclusion in myostatin”; the contents of each of which are incorporated herein in their entireties.

In some embodiments, an oligonucleotide that is useful for targeting MSTN is an oligonucleotide that promotes exon skipping of MSTN RNA sequences. In some embodiments, an oligonucleotide for targeting MSTN promotes exon skipping of exon 2. Skipping of exon 2 may lead to an improper out-of-phase splicing of exons 1 and 3. In some embodiments, an oligonucleotide for targeting MSTN targets a RNA splice junction, e.g., at intron 1/exon 2 or exon 2/intron 2.

Examples of oligonucleotides for promoting MSTN gene editing include Crispo, M. et. al. “Efficient Generation of Myostatin Knock-Out Sheep Using CRISPR/Cas9 Technology and Microinjection into Zygotes” PLoS One. 2015 Aug. 25; 10(8):e0136690; and Zhang, J. et. al. “Comparison of gene editing efficiencies of CRISPR/Cas9 and TALEN for generation of MSTN knock-out cashmere goats” Theriogenology. 2019 Jul. 1; 132:1-11.

In some embodiments, oligonucleotides may have a region of complementarity to a human MSTN gene sequence, for example, as provided below (Gene ID: 2660; NCBI Ref. No: NM_005259.3):

(SEQ ID NO. 147)
AGATTCACTGGTGTGGCAAGTTGTCTCTCAGACTGTACATGCATTAAAATTTTGCTTGGCATTA
CTCAAAAGCAAAAGAAAAGTAAAAGGAAGAAACAAGAACAAGAAAAAAGATTATATTGATTTTA
AAATCATGCAAAAACTGCAACTCTGTGTTTATATTTACCTGTTTATGCTGATTGTTGCTGGTCC
AGTGGATCTAAATGAGAACAGTGAGCAAAAAGAAAATGTGGAAAAAGAGGGGCTGTGTAATGCA
TGTACTTGGAGACAAAACACTAAATCTTCAAGAATAGAAGCCATTAAGATACAAATCCTCAGTA
AACTTCGTCTGGAAACAGCTCCTAACATCAGCAAAGATGTTATAAGACAACTTTTACCCAAAGC
TCCTCCACTCCGGGAACTGATTGATCAGTATGATGTCCAGAGGGATGACAGCAGCGATGGCTCT
TTGGAAGATGACGATTATCACGCTACAACGGAAACAATCATTACCATGCCTACAGAGTCTGATT
TTCTAATGCAAGTGGATGGAAAACCCAAATGTTGCTTCTTTAAATTTAGCTCTAAAATACAATA
CAATAAAGTAGTAAAGGCCCAACTATGGATATATTTGAGACCCGTCGAGACTCCTACAACAGTG
TTTGTGCAAATCCTGAGACTCATCAAACCTATGAAAGACGGTACAAGGTATACTGGAATCCGAT
CTCTGAAACTTGACATGAACCCAGGCACTGGTATTTGGCAGAGCATTGATGTGAAGACAGTGTT
GCAAAATTGGCTCAAACAACCTGAATCCAACTTAGGCATTGAAATAAAAGCTTTAGATGAGAAT
GGTCATGATCTTGCTGTAACCTTCCCAGGACCAGGAGAAGATGGGCTGAATCCGTTTTTAGAGG
TCAAGGTAACAGACACACCAAAAAGATCCAGAAGGGATTTTGGTCTTGACTGTGATGAGCACTC
AACAGAATCACGATGCTGTCGTTACCCTCTAACTGTGGATTTTGAAGCTTTTGGATGGGATTGG
ATTATCGCTCCTAAAAGATATAAGGCCAATTACTGCTCTGGAGAGTGTGAATTTGTATTTTTAC
AAAAATATCCTCATACTCATCTGGTACACCAAGCAAACCCCAGAGGTTCAGCAGGCCCTTGCTG
TACTCCCACAAAGATGTCTCCAATTAATATGCTATATTTTAATGGCAAAGAACAAATAATATAT
GGGAAAATTCCAGCGATGGTAGTAGACCGCTGTGGGTGCTCATGAGATTTATATTAAGCGTTCA
TAACTTCCTAAAACATGGAAGGTTTTCCCCTCAACAATTTTGAAGCTGTGAAATTAAGTACCAC
AGGCTATAGGCCTAGAGTATGCTACAGTCACTTAAGCATAAGCTACAGTATGTAAACTAAAAGG
GGGAATATATGCAATGGTTGGCATTTAACCATCCAAACAAATCATACAAGAAAGTTTTATGATT
TCCAGAGTTTTTGAGCTAGAAGGAGATCAAATTACATTTATGTTCCTATATATTACAACATCGG
CGAGGAAATGAAAGCGATTCTCCTTGAGTTCTGATGAATTAAAGGAGTATGCTTTAAAGTCTAT
TTCTTTAAAGTTTTGTTTAATATTTACAGAAAAATCCACATACAGTATTGGTAAAATGCAGGAT
TGTTATATACCATCATTCGAATCATCCTTAAACACTTGAATTTATATTGTATGGTAGTATACTT
GGTAAGATAAAATTCCACAAAAATAGGGATGGTGCAGCATATGCAATTTCCATTCCTATTATAA
TTGACACAGTACATTAACAATCCATGCCAACGGTGCTAATACGATAGGCTGAATGTCTGAGGCT
ACCAGGTTTATCACATAAAAAACATTCAGTAAAATAGTAAGTTTCTCTTTTCTTCAGGTGCATT
TTCCTACACCTCCAAATGAGGAATGGATTTTCTTTAATGTAAGAAGAATCATTTTTCTAGAGGT
TGGCTTTCAATTCTGTAGCATACTTGGAGAAACTGCATTATCTTAAAAGGCAGTCAAATGGTGT
TTGTTTTTATCAAAATGTCAAAATAACATACTTGGAGAAGTATGTAATTTTGTCTTTGGAAAAT
TACAACACTGCCTTTGCAACACTGCAGTTTTTATGGTAAAATAATAGAAATGATCGACTCTATC
AATATTGTATAAAAAGACTGAAACAATGCATTTATATAATATGTATACAATATTGTTTTGTAAA
TAAGTGTCTCCTTTTTTATTTACTTTGGTATATTTTTACACTAAGGACATTTCAAATTAAGTAC
TAAGGCACAAAGACATGTCATGCATCACAGAAAAGCAACTACTTATATTTCAGAGCAAATTAGC
AGATTAAATAGTGGTCTTAAAACTCCATATGTTAATGATTAGATGGTTATATTACAATCATTTT
ATATTTTTTTACATGATTAACATTCACTTATGGATTCATGATGGCTGTATAAAGTGAATTTGAA
ATTTCAATGGTTTACTGTCATTGTGTTTAAATCTCAACGTTCCATTATTTTAATACTTGCAAAA
ACATTACTAAGTATACCAAAATAATTGACTCTATTATCTGAAATGAAGAATAAACTGATGCTAT
CTCAACAATAACTGTTACTTTTATTTTATAATTTGATAATGAATATATTTCTGCATTTATTTAC
TTCTGTTTTGTAAATTGGGATTTTGTTAATCAAATTTATTGTACTATGACTAAATGAAATTATT
TCTTACATCTAATTTGTAGAAACAGTATAAGTTATATTAAAGTGTTTTCACATTTTTTTGAAAG
ACA

In some embodiments, oligonucleotides may have a region of complementarity to a mouse MSTN gene sequence, for example, as provided below (Gene ID: 17700; NCBI Ref. No: NM_010834.3):

(SEQ ID NO: 148)
AGGACTCCCTGGCGTGGCAGGTTGTCTCTCGGACGGTACATGCACTAATATTTCACTTGGCATT
ACTCAAAAGCAAAAAGAAGAAATAAGAACAAGGGAAAAAAAAAGATTGTGCTGATTTTTAAAAT
GATGCAAAAACTGCAAATGTATGTTTATATTTACCTGTTCATGCTGATTGCTGCTGGCCCAGTG
GATCTAAATGAGGGCAGTGAGAGAGAAGAAAATGTGGAAAAAGAGGGGCTGTGTAATGCATGTG
CGTGGAGACAAAACACGAGGTACTCCAGAATAGAAGCCATAAAAATTCAAATCCTCAGTAAGCT
GCGCCTGGAAACAGCTCCTAACATCAGCAAAGATGCTATAAGACAACTTCTGCCAAGAGCGCCT
CCACTCCGGGAACTGATCGATCAGTACGACGTCCAGAGGGATGACAGCAGTGATGGCTCTTTGG
AAGATGACGATTATCACGCTACCACGGAAACAATCATTACCATGCCTACAGAGTCTGACTTTCT
AATGCAAGCGGATGGCAAGCCCAAATGTTGCTTTTTTAAATTTAGCTCTAAAATACAGTACAAC
AAAGTAGTAAAAGCCCAACTGTGGATATATCTCAGACCCGTCAAGACTCCTACAACAGTGTTTG
TGCAAATCCTGAGACTCATCAAACCCATGAAAGACGGTACAAGGTATACTGGAATCCGATCTCT
GAAACTTGACATGAGCCCAGGCACTGGTATTTGGCAGAGTATTGATGTGAAGACAGTGTTGCAA
AATTGGCTCAAACAGCCTGAATCCAACTTAGGCATTGAAATCAAAGCTTTGGATGAGAATGGCC
ATGATCTTGCTGTAACCTTCCCAGGACCAGGAGAAGATGGGCTGAATCCCTTTTTAGAAGTCAA
GGTGACAGACACACCCAAGAGGTCCCGGAGAGACTTTGGGCTTGACTGCGATGAGCACTCCACG
GAATCCCGGTGCTGCCGCTACCCCCTCACGGTCGATTTTGAAGCCTTTGGATGGGACTGGATTA
TCGCACCCAAAAGATATAAGGCCAATTACTGCTCAGGAGAGTGTGAATTTGTGTTTTTACAAAA
ATATCCGCATACTCATCTTGTGCACCAAGCAAACCCCAGAGGCTCAGCAGGCCCTTGCTGCACT
CCGACAAAAATGTCTCCCATTAATATGCTATATTTTAATGGCAAAGAACAAATAATATATGGGA
AAATTCCAGCCATGGTAGTAGACCGCTGTGGGTGCTCATGAGCTTTGCATTAGGTTAGAAATTT
CCCAAGTCATGGAAGGTCTTCCCCTCAATTTCGAAACTGTGAATTCAAGCACCACAGGCTGTAG
GCCTTGAGTATGCTCTAGTAACGTAAGCACAAGCTACAGTGTATGAACTAAAAGAGAGAATAGA
TGCAATGGTTGGCATTCAACCACCAAAATAAACCATACTATAGGATGTTGTATGATTTCCAGAG
TTTTTGAAATAGATGGAGATCAAATTACATTTATGTCCATATATGTATATTACAACTACAATCT
AGGCAAGGAAGTGAGAGCACATCTTGTGGTCTGCTGAGTTAGGAGGGTATGATTAAAAGGTAAA
GTCTTATTTCCTAACAGTTTCACTTAATATTTACGGAAGAATCTATATGTAGCCTTTGTAAAGT
GTAGGATTGTTATCATTTAAAAACATCATGTACACTTATATTTGTATTGTATACTTGGTAAGAT
AAAATTCCACAAAGTAGGAATGGGGCCTTACATACACATTGCCATTCCTATTATAATTGGACAA
TCCACCACGGTGCTAATGCAGTGCTGAATGGCTCCTACTGGACCTCTCGATAGAACACTCTACA
AAGTACGAGTCTCTCTCTCCCTTCCAGGTGCATCTCCACACACACAGCACTAAGTGTTCAATGC
ATTTTCTTTAAGGAAAGAAGAATCTTTTTTTCTAGAGGTCAACTTTCAGTCAACTCTAGCACAG
CGGGAGTGACTGCTGCATCTTAAAAGGCAGCCAAACAGTATTCATTTTTTAATCTAAATTTCAA
AATCACTGTCTGCCTTTATCACATGGCAATTTTGTGGTAAAATAATGGAAATGACTGGTTCTAT
CAATATTGTATAAAAGACTCTGAAACAATTACATTTATATAATATGTATACAATATTGTTTTGT
AAATAAGTGTCTCCTTTTATATTTACTTTGGTATATTTTTACACTAATGAAATTTCAAATCATT
AAAGTACAAAGACATGTCATGTATCACAAAAAAGGTGACTGCTTCTATTTCAGAGTGAATTAGC
AGATTCAATAGTGGTCTTAAAACTCTGTATGTTAAGATTAGAAGGTTATATTACAATCAATTTA
TGTATTTTTTACATTATCAACATTCACTTATGGTTTCATGGTGGCTGTATCTATGAATGTGGCT
CCCAGTCAAATTTCAATGCCCCACCATTTTAAAAATTACAAGCATTACTAAACATACCAACATG
TATCTAAAGAAATACAAATATGGTATCTCAATAACAGCTACTTTTTTATTTTATAATTTGACAA
TGAATACATTTCTTTTATTTACTTCAGTTTTATAAATTGGAACTTTGTTTATCAAATGTATTGT
ACTCATAGCTAAATGAAATTATTTCTTACATAAAAATGTGTAGAAACTATAAATTAAAGTGTTT
TCACATTTTTGAAAGGC

In some embodiments, the oligonucleotide may have region of complementarity to a mutant form of MSTN, for example as reported in as in Schuelke, M. et al., “Myostatin Mutation Associated with Gross Muscle Hypertrophy in a Child” N Engl J Med 2004; 350:2682-2688, the contents of which are incorporated herein by reference in its entirety.

In some embodiments, an oligonucleotide comprises a region of complementarity to an MSTN sequence as set forth in SEQ ID NO: 147 or SEQ ID NO: 148. In some embodiments, the oligonucleotide comprises a region of complementarity that is at least 80%, 85%, 90%, 95%, 96%, 97%, 98%, 99%, or 100% complementary to an MSTN sequence as set forth in SEQ ID NO: 147 or SEQ ID NO: 148. In some embodiments, the oligonucleotide comprises a sequence that has at least 10, 11, 12, 13, 14, 15, 16, 17, 18, or 19 consecutive nucleotides that are perfectly complementary to an MSTN sequence as set forth in SEQ ID NO: 147 or SEQ ID NO: 148. In some embodiments, an oligonucleotide may comprise a sequence that targets (e.g., is complementary to) an RNA version (i.e., wherein the T's are replaced with U's) of an MSTN sequence as set forth in SEQ ID NO: 147 or SEQ ID NO: 148. In some embodiments, the oligonucleotide comprises a sequence that is complementary (e.g., at least 80%, 85%, 90%, 95%, 96%, 97%, 98%, 99%, or 100% complementary) to an RNA version of an MSTN sequence as set forth in SEQ ID NO: 147 or SEQ ID NO: 148. In some embodiments, the oligonucleotide comprises a sequence that has at least 10, 11, 12, 13, 14, 15, 16, 17, 18, or 19 consecutive nucleotides that are perfectly complementary to an RNA version of an MSTN sequence as set forth in SEQ ID NO: 147 or SEQ ID NO: 148.

In some embodiments, an MSTN-targeting oligonucleotide comprises an antisense strand that comprises at least 10, 11, 12, 13, 14, 15, 16, 17, 18, or 19 consecutive nucleotides of a sequence comprising any one of SEQ ID NOs: 197-220. In some embodiments, an MSTN-targeting oligonucleotide comprises an antisense strand that comprises any one of SEQ ID NO: 197-220. In some embodiments, an oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 197-220.

In some embodiments, an MSTN-targeting oligonucleotide comprises an antisense strand that targets an MSTN sequence comprising any one of SEQ ID NO: 149-196. In some embodiments, an oligonucleotide comprises an antisense strand comprising at least 10, 11, 12, 13, 14, 15, 16, 17, 18, or 19 nucleotides (e.g., consecutive nucleotides) that are complementary to an MSTN sequence comprising any one of SEQ ID NO: 149-196. In some embodiments, an MSTN-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NO: 149-196.

In some embodiments, an MSTN-targeting oligonucleotide comprises an antisense strand that comprises a region of complementarity to a target sequence as set forth in any one of SEQ ID NOs: 149-196. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, an MSTN-targeting oligonucleotide further comprises a sense strand that hybridizes to the antisense strand to form a double stranded siRNA. In some embodiments, the MSTN-targeting oligonucleotide comprises an antisense strand that comprises the nucleotide sequence of any one of SEQ ID NOs: 197-220. In some embodiments, the MSTN-targeting oligonucleotide further comprises a sense strand that comprises the nucleotide sequence of any one of SEQ ID NOs: 173-196.

In some embodiments, the MSTN-targeting oligonucleotide is a double stranded oligonucleotide (e.g., an siRNA) comprising an antisense strand that comprises the nucleotide sequence of any one of SEQ ID NOs: 197-220 and a sense strand that hybridizes to the antisense strand and comprises the nucleotide sequence of any one of SEQ ID NOs: 173-196, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the MSTN-targeting oligonucleotide is a double stranded oligonucleotide (e.g., an siRNA) comprising an antisense strand that comprises the nucleotide sequence of any one of SEQ ID NOs: 197-220 and a sense strand that hybridizes to the antisense strand and comprises the nucleotide sequence of any one of SEQ ID NOs: 173-196, wherein each nucleoside in the antisense strand and/or (e.g., and) each nucleoside in the sense strand is a 2′-modified nucleoside selected from 2′-O-Me and 2′-F modified nucleosides.

In some embodiments, the MSTN-targeting oligonucleotide is a double stranded oligonucleotide (e.g., an siRNA) comprising an antisense strand that comprises the nucleotide sequence of any one of SEQ ID NOs: 197-220 and a sense strand that hybridizes to the antisense strand and comprises the nucleotide sequence of any one of SEQ ID NOs: 173-196, wherein each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the MSTN-targeting oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates a phosphorothioate internucleoside linkage; and the absence of “*” between two nucleosides indicates a phosphodiester internucleoside linkage.

In some embodiments, the sense strand of the MSTN-targeting oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicates a phosphodiester internucleoside linkage.

In some embodiments, the antisense strand of the MSTN-targeting oligonucleotide is selected from the modified version of SEQ ID NOs: 197-220 listed in Table 10. In some embodiments, the sense strand of the MSTN-targeting oligonucleotide is selected from the modified version of SEQ ID NOs: 173-196 listed in Table 10. In some embodiments, the MSTN-targeting oligonucleotide is an siRNA selected from the siRNAs listed in Table 10.

TABLE 8
MSTN Target Sequences

| Corresponding | | |
|---|---|---|
| nucleotides in | MSTN | |
| NM_005259.3 | Target Sequence | SEQ |
| (SEQ ID NO: 300) | (5′ to 3′) | ID NO: |
| 448-466 | TTTGGAAGATGACGATTAT | 149 |
| 450-468 | TGGAAGATGACGATTATCA | 150 |
| 454-472 | AGATGACGATTATCACGCT | 151 |
| 482-500 | ACAATCATTACCATGCCTA | 152 |
| 630-644 | CTACAACAGTGTTTGTGCA | 153 |
| 632-650 | ACAACAGTGTTTGTGCAAA | 154 |
| 671-679 | ATGAAAGACGGTACAAGGT | 155 |
| 697-715 | AATCCGATCTCTGAAACTT | 156 |
| 699-717 | TCCGATCTCTGAAACTTGA | 157 |
| 754-772 | TGTGAAGACAGTGTTGCAA | 158 |
| 760-788 | GACAGTGTTGCAAAATTGG | 159 |
| 762-780 | CAGTGTTGCAAAATTGGCT | 160 |
| 766-784 | GTTGCAAAATTGGCTCAAA | 161 |
| 788-806 | CCTGAATCCAACTTAGGCA | 162 |
| 789-807 | CTGAATCCAACTTAGGCAT | 163 |
| 792-810 | AATCCAACTTAGGCATTGA | 164 |
| 793-811 | ATCCAACTTAGGCATTGAA | 165 |
| 846-864 | CTGTAACCTTCCCAGGACC | 166 |
| 865-883 | AGGAGAAGATGGGCTGAAT | 167 |
| 1181-1199 | ATGCTATATTTTAATGGCA | 168 |
| 1185-1203 | TATATTTTAATGGCAAAGA | 169 |
| 1201-1219 | AGAACAAATAATATATGGG | 170 |
| 1202-1220 | GAACAAATAATATATGGGA | 171 |
| 1203-1221 | AACAAATAATATATGGGAA | 172 |

*The target sequences contain Ts, but binding to RNA and/or DNA is contemplated.

In some embodiments, an oligonucleotide may comprise or consist of any sequence as provided in Table 9.

TABLE 9
Oligonucleotide sequences for targeting MSTN

| Passenger Strand/ | | Guide Strand/ | |
|---|---|---|---|
| Sense Strand | SEQ ID | Antisense Strand | SEQ |
| (RNA) (5′ to 3′) | NO: | (RNA) (5′ to 3′) | ID NO: |
| UCUUUGGAAGAUGACGAUUAU | 173 | AUAAUCGUCAUCUUCCAAAGAGC | 197 |
| UUUGGAAGAUGACGAUUAUCA | 174 | UGAUAAUCGUCAUCUUCCAAAGA | 198 |
| GAAGAUGACGAUUAUCACGCU | 175 | AGCGUGAUAAUCGUCAUCUUCCA | 199 |
| AAACAAUCAUUACCAUGCCUA | 176 | UAGGCAUGGUAAUGAUUGUUUCC | 200 |
| UCCUACAACAGUGUUUGUGCA | 177 | UGCACAAACACUGUUGUAGGAGU | 201 |
| CUACAACAGUGUUUGUGCAAA | 178 | UUUGCACAAACACUGUUGUAGGA | 202 |
| CUAUGAAAGACGGUACAAGGU | 179 | ACCUUGUACCGUCUUUCAUAGGU | 203 |
| GGAAUCCGAUCUCUGAAACUU | 180 | AAGUUUCAGAGAUCGGAUUCCAG | 204 |
| AAUCCGAUCUCUGAAACUUGA | 181 | UCAAGUUUCAGAGAUCGGAUUCC | 205 |
| GAUGUGAAGACAGUGUUGCAA | 182 | UUGCAACACUGUCUUCACAUCAA | 206 |
| AAGACAGUGUUGCAAAAUUGG | 183 | CCAAUUUUGCAACACUGUCUUCA | 207 |
| GACAGUGUUGCAAAAUUGGCU | 184 | AGCCAAUUUUGCAACACUGUCUU | 208 |
| GUGUUGCAAAAUUGGCUCAAA | 185 | UUUGAGCCAAUUUUGCAACACUG | 209 |
| AACCUGAAUCCAACUUAGGCA | 186 | UGCCUAAGUUGGAUUCAGGUUGU | 210 |
| ACCUGAAUCCAACUUAGGCAU | 187 | AUGCCUAAGUUGGAUUCAGGUUG | 211 |
| UGAAUCCAACUUAGGCAUUGA | 188 | UCAAUGCCUAAGUUGGAUUCAGG | 212 |
| GAAUCCAACUUAGGCAUUGAA | 189 | UUCAAUGCCUAAGUUGGAUUCAG | 213 |
| UGCUGUAACCUUCCCAGGACC | 190 | GGUCCUGGGAAGGUUACAGCAAG | 214 |
| CCAGGAGAAGAUGGGCUGAAU | 191 | AUUCAGCCCAUCUUCUCCUGGUC | 215 |
| AUAUGCUAUAUUUUAAUGGCA | 192 | UGCCAUUAAAAUAUAGCAUAUUA | 216 |
| GCUAUAUUUUAAUGGCAAAGA | 193 | UCUUUGCCAUUAAAAUAUAGCAU | 217 |
| AAAGAACAAAUAAUAUAUGGG | 194 | CCCAUAUAUUAUUUGUUCUUUGC | 218 |
| AAGAACAAAUAAUAUAUGGGA | 195 | UCCCAUAUAUUAUUUGUUCUUUG | 219 |
| AGAACAAAUAAUAUAUGGGAA | 196 | UUCCCAUAUAUUAUUUGUUCUUU | 220 |

In some embodiments, an oligonucleotide is a modified oligonucleotide as provided in Table 10, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 10
Modified Oligonucleotides for targeting MSTN

| | Modified Passenger | | Modified Guide | |
|---|---|---|---|---|
| | Strand/Sense Strand | | Strand/Antisense | |
| | (RNA) | SEQ ID | Strand (RNA) | SEQ ID |
| siRNA # | (5′ to 3′) | NO: | (5′ to 3′) | NO: |
| hsMSTN-1 | mUmCfUmUfUmGfGmAfA | 173 | fAfUmAfAmUfCmGfUmCfAmUfCm | 197 |
| | mGfAmUfGmAfCmGfAmU | | UfUmCfCmAfAmAfGmA*fG*mC | |
| | fUmAfU | | | |
| hsMSTN-5 | mUmUfUmGfGmAfAmGfA | 174 | fUfGmAfUmAfAmUfCmGfUmCfAm | 198 |
| | mUfGmAfCmGfAmUfUmA | | UfCmUfUmCfCmAfAmA*fG*mA | |
| | fUmCfA | | | |
| hsMSTN-2 | mGmAfAmGfAmUfGmAfC | 175 | fAfGmCfGmUfGmAfUmAfAmUfCm | 199 |
| | mGfAmUfUmAfUmCfAmCf | | GfUmCfAmUfCmUfUmC*fC*mA | |
| | GmCfU | | | |
| hsMSTN-6 | mAmAfAmCfAmAfUmCfA | 176 | fUfAmGfGmCfAmUfGmGfUmAfAm | 200 |
| | mUfUmAfCmCfAmUfGmCf | | UfGmAfUmUfGmUfUmU*fC*mC | |
| | CmUfA | | | |
| hsMSTN-7 | mUmCfCmUfAmCfAmAfC | 177 | fUfGmCfAmCfAmAfAmCfAmCfUm | 201 |
| | mAfGmUfGmUfUmUfGmU | | GfUmUfGmUfAmGfGmA*fG*mU | |
| | fGmCfA | | | |
| hsMSTN-8 | mCmUfAmCfAmAfCmAfG | 178 | fUfUmUfGmCfAmCfAmAfAmCfAm | 202 |
| | mUfGmUfUmUfGmUfGmC | | CfUmGfUmUfGmUfAmG*fG*mA | |
| | fAmAfA | | | |
| hsMSTN-9 | mCmUfAmUfGmAfAmAfG | 179 | fAfCmCfUmUfGmUfAmCfCmGfUm | 203 |
| | mAfCmGfGmUfAmCfAmAf | | CfUmUfUmCfAmUfAmG*fG*mU | |
| | GmGfU | | | |
| hsMSTN-10 | mGmGfAmAfUmCfCmGfA | 180 | fAfAmGfUmUfUmCfAmGfAmGfAm | 204 |
| | mUfCmUfCmUfGmAfAmAf | | UfCmGfGmAfUmUfCmC*fA*mG | |
| | CmUfU | | | |
| hsMSTN-11 | mAmAfUmCfCmGfAmUfC | 181 | fUfCmAfAmGfUmUfUmCfAmGfAm | 205 |
| | mUfCmUfGmAfAmAfCmUf | | GfAmUfCmGfGmAfUmU*fC*mC | |
| | UmGfA | | | |
| hsMSTN-12 | mGmAfUmGfUmGfAmAfG | 182 | fUfUmGfCmAfAmCfAmCfUmGfUm | 206 |
| | mAfCmAfGmUfGmUfUmG | | CfUmUfCmAfCmAfUmC*fA*mA | |
| | fCmAfA | | | |
| hsMSTN-13 | mAmAfGmAfCmAfGmUfG | 183 | fCfCmAfAmUfUmUfUmGfCmAfAm | 207 |
| | mUfUmGfCmAfAmAfAmU | | CfAmCfUmGfUmCfUmU*fC*mA | |
| | fUmGfG | | | |
| hsMSTN-3 | mGmAfCmAfGmUfGmUfU | 184 | fAfGmCfCmAfAmUfUmUfUmGfCm | 208 |
| | mGfCmAfAmAfAmUfUmG | | AfAmCfAmCfUmGfUmC*fU*mU | |
| | fGmCfU | | | |
| hsMSTN-4 | mGmUfGmUfUmGfCmAfA | 185 | fUfUmUfGmAfGmCfCmAfAmUfUm | 209 |
| | mAfAmUfUmGfGmCfUmCf | | UfUmGfCmAfAmCfAmC*fU*mG | |
| | AmAfA | | | |
| hsMSTN-14 | mAmAfCmCfUmGfAmAfU | 186 | fUfGmCfCmUfAmAfGmUfUmGfGm | 210 |
| | mCfCmAfAmCfUmUfAmGf | | AfUmUfCmAfGmGfUmU*fG*mU | |
| | GmCfA | | | |
| hsMSTN-15 | mAmCfCmUfGmAfAmUfC | 187 | fAfUmGfCmCfUmAfAmGfUmUfGm | 211 |
| | mCfAmAfCmUfUmAfGmGf | | GfAmUfUmCfAmGfGmU*fU*mG | |
| | CmAfU | | | |
| hsMSTN-16 | mUmGfAmAfUmCfCmAfA | 188 | fUfCmAfAmUfGmCfCmUfAmAfGm | 212 |
| | mCfUmUfAmGfGmCfAmUf | | UfUmGfGmAfUmUfCmA*fG*mG | |
| | UmGfA | | | |
| hsMSTN-17 | mGmAfAmUfCmCfAmAfC | 189 | fUfUmCfAmAfUmGfCmCfUmAfAm | 213 |
| | mUfUmAfGmGfCmAfUmU | | GfUmUfGmGfAmUfUmC*fA*mG | |
| | fGmAfA | | | |
| hsMSTN-18 | mUmGfCmUfGmUfAmAfC | 190 | fGfGmUfCmCfUmGfGmGfAmAfGm | 214 |
| | mCfUmUfCmCfCmAfGmGf | | GfUmUfAmCfAmGfCmA*fA*mG | |
| | AmCfC | | | |
| hsMSTN-19 | mCmCfAmGfGmAfGmAfA | 191 | fAfUmUfCmAfGmCfCmCfAmUfCm | 215 |
| | mGfAmUfGmGfGmCfUmG | | UfUmCfUmCfCmUfGmG*fU*mC | |
| | fAmAfU | | | |
| hsMSTN-20 | mAmUfAmUfGmCfUmAfU | 192 | fUfGmCfCmAfUmUfAmAfAmAfUm | 216 |
| | mAfUmUfUmUfAmAfUmG | | AfUmAfGmCfAmUfAmU*fU*mA | |
| | fGmCfA | | | |
| hsMSTN-21 | mGmCfUmAfUmAfUmUfU | 193 | fUfCmUfUmUfGmCfCmAfUmUfAm | 217 |
| | mUfAmAfUmGfGmCfAmA | | AfAmAfUmAfUmAfGmC*fA*mU | |
| | fAmGfA | | | |
| hsMSTN-22 | mAmAfAmGfAmAfCmAfA | 194 | fCfCmCfAmUfAmUfAmUfUmAfUm | 218 |
| | mAfUmAfAmUfAmUfAmU | | UfUmGfUmUfCmUfUmU*fG*mC | |
| | fGmGfG | | | |
| hsMSTN-23 | mAmAfGmAfAmCfAmAfA | 195 | fUfCmCfCmAfUmAfUmAfUmUfAm | 219 |
| | mUfAmAfUmAfUmAfUmG | | UfUmUfGmUfUmCfUmU*fU*mG | |
| | fGmGfA | | | |
| hsMSTN-24 | mAmGfAmAfCmAfAmAfU | 196 | fUfUmCfCmCfAmUfAmUfAmUfUm | 220 |
| | mAfAmUfAmUfAmUfGmG | | AfUmUfUmGfUmUfCmU*fU*mU | |
| | fGmAfA | | | |

b. INHBA Oligonucleosides

Examples of oligonucleotides useful for targeting INHBA are provided in Tada et. al., “Differential expression and cellular localization of activin and inhibin mRNA in the rainbow trout ovary and testis” Gen Comp Endocrinol. 2002 January; 125(1):142-9.; U.S. Pat. No. 10,260,068, issued on Apr. 16, 2019, and entitled “Prophylactic agent and therapeutic agent for fibrodysplasia ossificans progressiva”; Carlton, A L et. al. “Small molecule inhibition of the CBFβ/RUNX interaction decreases ovarian cancer growth and migration through alterations in genes related to epithelial-to-mesenchymal transition” Gynecol Oncol. 2018 May; 149(2):350-360.; and Takabe, K. et al. “Interruption of activin A autocrine regulation by antisense oligodeoxynucleotides accelerates liver tumor cell proliferation” Endocrinology. 1999 Jul; 140(7):3125-32.; the contents of each of which are incorporated herein in their entireties.

In some embodiments, oligonucleotides may have a region of complementarity to a human INHBA sequence, for example, as provided below (Gene ID: 3624; NCBI Ref. No: NM_002192.4):

(SEQ ID NO: 269)
ACAGTGCCAATACCATGAAGAGGAGCTCAGACAGCTCTTACCACATGATACAAGAGCCGGCTGG
TGGAAGAGTGGGGACCAGAAAGAGAATTTGCTGAAGAGGAGAAGGAAAAAAAAAACACCAAAAA
AAAAAATAAAAAAATCCACACACACAAAAAAACCTGCGCGTGAGGGGGGAGGAAAAGCAGGGCC
TTTTAAAAAGGCAATCACAACAACTTTTGCTGCCAGGATGCCCTTGCTTTGGCTGAGAGGATTT
CTGTTGGCAAGTTGCTGGATTATAGTGAGGAGTTCCCCCACCCCAGGATCCGAGGGGCACAGCG
CGGCCCCCGACTGTCCGTCCTGTGCGCTGGCCGCCCTCCCAAAGGATGTACCCAACTCTCAGCC
AGAGATGGTGGAGGCCGTCAAGAAGCACATTTTAAACATGCTGCACTTGAAGAAGAGACCCGAT
GTCACCCAGCCGGTACCCAAGGCGGCGCTTCTGAACGCGATCAGAAAGCTTCATGTGGGCAAAG
TCGGGGAGAACGGGTATGTGGAGATAGAGGATGACATTGGAAGGAGGGCAGAAATGAATGAACT
TATGGAGCAGACCTCGGAGATCATCACGTTTGCCGAGTCAGGAACAGCCAGGAAGACGCTGCAC
TTCGAGATTTCCAAGGAAGGCAGTGACCTGTCAGTGGTGGAGCGTGCAGAAGTCTGGCTCTTCC
TAAAAGTCCCCAAGGCCAACAGGACCAGGACCAAAGTCACCATCCGCCTCTTCCAGCAGCAGAA
GCACCCGCAGGGCAGCTTGGACACAGGGGAAGAGGCCGAGGAAGTGGGCTTAAAGGGGGAGAGG
AGTGAACTGTTGCTCTCTGAAAAAGTAGTAGACGCTCGGAAGAGCACCTGGCATGTCTTCCCTG
TCTCCAGCAGCATCCAGCGGTTGCTGGACCAGGGCAAGAGCTCCCTGGACGTTCGGATTGCCTG
TGAGCAGTGCCAGGAGAGTGGCGCCAGCTTGGTTCTCCTGGGCAAGAAGAAGAAGAAAGAAGAG
GAGGGGGAAGGGAAAAAGAAGGGCGGAGGTGAAGGTGGGGCAGGAGCAGATGAGGAAAAGGAGC
AGTCGCACAGACCTTTCCTCATGCTGCAGGCCCGGCAGTCTGAAGACCACCCTCATCGCCGGCG
TCGGCGGGGCTTGGAGTGTGATGGCAAGGTCAACATCTGCTGTAAGAAACAGTTCTTTGTCAGT
TTCAAGGACATCGGCTGGAATGACTGGATCATTGCTCCCTCTGGCTATCATGCCAACTACTGCG
AGGGTGAGTGCCCGAGCCATATAGCAGGCACGTCCGGGTCCTCACTGTCCTTCCACTCAACAGT
CATCAACCACTACCGCATGCGGGGCCATAGCCCCTTTGCCAACCTCAAATCGTGCTGTGTGCCC
ACCAAGCTGAGACCCATGTCCATGTTGTACTATGATGATGGTCAAAACATCATCAAAAAGGACA
TTCAGAACATGATCGTGGAGGAGTGTGGGTGCTCATAGAGTTGCCCAGCCCAGGGGGAAAGGGA
GCAAGAGTTGTCCAGAGAAGACAGTGGCAAAATGAAGAAATTTTTAAGGTTTCTGAGTTAACCA
GAAAAATAGAAATTAAAAACAAAACAAAAAAAAAAACAAAAAAAAACAAAAGTAAATTAAAAAC
AAAACCTGATGAACAGATGAAGGAAGATGTGGAAAAAATCCTTAGCCAGGGCTCAGAGATGAAG
CAGTGAAAGAGACAGGAATTGGGAGGGAAAGGGAGAATGGTGTACCCTTTATTTCTTCTGAAAT
CACACTGATGACATCAGTTGTTTAAACGGGGTATTGTCCTTTCCCCCCTTGAGGTTCCCTTGTG
AGCCTTGAATCAACCAATCTAGTCTGCAGTAGTGTGGACTAGAACAACCCAAATAGCATCTAGA
AAGCCATGAGTTTGAAAGGGCCCATCACAGGCACTTTCCTACCCAATTACCCAGGTCATAAGGT
ATGTCTGTGTGACACTTATCTCTGTGTATATCAGCATACACACACACACACACACACACACACA
CACACACAGGCATTTCCACACATTACATATATACACATACTGGTAAAAGAACAATCGTGTGCAG
GTGGTCACACTTCCTTTTTCTGTACCACTTTTGCAACAAAACAAAACAAACAACATTAAAAAAT
TGAGAACAAGTATGGAAAGAATGAAAGATCAAGGAAAAAAGAATACCAAGTTACATTTCGTTAA
GGTGCTTATGATCTTAGAACTATGCAACCTAATAGGTTTGAAACTGTTTACCTGAGAGAGAACA
AAAAGAGAGACTTTTTTGTATTGGAAGTAATCTGATTAATTTTTATTTTCTTCAAGGAGAGATA
CTTGAAAGGAATATGTTTGTCCATCTGTTGGATCCAAACATTTCTATATTTTGTAAATGTTGTT
GTTGTTTTTTTTTTAATCGTTTACTATTTGCACTACAATGGTGTTTGACCTGTCTAATCCTTAT
TTAACAAGTATTTTCTTTGGTTGGGGGTGGGGGTGGGGTTTAAGAGCTGCACTTAATGTGAGCT
ATAAAAGAACTGCTACAGCACACAAAATAGCTATTTTTATTATTATAATTATAATTATTATTAT
TATTTTGTACCTTAAAAAATAGACACATACACCAAAGACATTTGTGTGAGCCTTTAAACAGTCT
GTCTGTGGTTGGTATCATTCACCATCAATGAGTCAGGGGTTGGGATTCAAGGTTGAGTAGTGTG
GATTGTGTTCAGGCTTAAAAGACCTGAGAAGTTTGGTTTTTGACTCCTTTTACATCCATGAAAC
AGGACATTTCATACTGGATGTACAGTAGTTGTACACTGTTGGATATCAAGTTCAATCAAATTCA
TGGAACTACATGCTTGTATGTGTATATATACATTGCTTGTGCATATGCATATCTGTATGTATAT
ATACATGTATTGTACCATGTCCATACACATTTTAAGCACTTCAGGCTGTCATTTTTTAATGTTC
TTAAAGCAATGAATGTTTGTGTGCAAAACACAGTATTTTTAAGAAGGATAGGCTATAGTTTTTG
CTTTTACTCTGAACTAGGTGGGCGCATTTCAAAAATTCGGATGGGAAAAAGCCTGGAAATTCCA
GTGAATATTCAGCAAGGCCCTCTTTCATTGTACAGGGATCAAATTTCCTCCTCTTTTTTGTGCC
CCCTCCCACTTCTACAAGTTATCCCCTGTGGGGAAAACAGGATGATAATCAAAACTCTGGGCTG
ATGTTTTTCCAACTTAGTGTCTATTGGAATCAATCTTAAATCAGAAGCTTTTTCAGAAAAATAA
TATTTAGGCCAGAATTAGAGTTGAGTGTATTTTTTAAAAATGATTAAGGCTTGGTTGTGAGAAA
TATTACCTGTACCAGCTGGGAAAAATAATGTCATCACTAACTAAAAGATAATTAATTTGAGAGA
AAGTGTTAAGAGAGGGAGAGTAAGGAAGAGAACAGTTAAGAGGAGGCAGAGGTGAGGGCAGTAG
TAAAAATCTCTAAAATTTTAATTTACAGCCAAAATTCTTCATGTGTAAATTTGTATTGATTCAG
ATGCAGAAATGAAAAAAAAACACCTTTGTTTTATAAATATCAAAGTACATGCTTAAAGCCAAGT
TTTTATCTAGTTTATTCTAGTACTTAGCTTGCCTGGAATAGCTAATAAATTATTCATGTATGTG
CTTTTGAAAATCCAGAGCCCTATTTTTACACACTTGTGTGAAGTTGGCAAACATTTTGAAAAAT
GGAAAAAAGTTTCTAATAATTGGGAACAATTACATTAATTAATATTTTGTAAAATATTGAAGCT
TTTAGCCCTATGTCAATTTGTAGATTAAAATAAATTAATTATAGGAAAGGAAGATAACAGTGAG
AAACCAAACATTACAAAAGGTGGTTTAGCTCTCCTTGAAAAATATACTAAGTTGGTATACTATA
ACACTTGGCTATATGTAGGCAATGTCACTACTGGGCAAATACACTTACTGTGTTCTAGAGGCAG
CCCTTTCTTATGCAGAAAATACAATACGCACTGCATGAGAAGCTTGAGAGTGGATTCTAATCCA
GGTCTGTCGACCTTGGATATCATGCATGTGGGAAGGTGGGTGTGGTGAGAAAAGTTTTAAGGCA
AGAGTAGATGGCCATGTTCAACTTTACAAAATTTCTTGGAAAACTGGCAGTATTTTGAACTGCA
TCTTCTTTGGTACCGGAACCTGCAGAAACAGTGTGAGAAATTAAGTCCTGGTTCACTGCGCAGT
AGCAAAGATGGTCAAGGCCATGGAAAAAGCAGAAATTTACCAAGAAAGCTGATACCCATGTATA
GTTCCCACTCATCTCAAATACATCTGCTATCTTTTTAAGCTAAGTCCTAGACATATCGGGGATA
ACATGGGGGTTGATTAGTGACCACAGTTATCAGAAGCAGAGAAATGTAATTCCATATTTTATTT
GAAACTTATTCCATATTTTAATTGGATATTGAGTGATTGGGTTATCAAACACCCACAAACTTTA
ATTTTGTTAAATTTATATGGCTTTGAAATAGAAGTATAAGTTGCTACCATTTTTTGATAACATT
GAAAGATAGTATTTTACCATCTTTAATCATCTTGGAAAATACAAGTCCTGTGAACAACCACTCT
TTCACCTAGCAGCATGAGGCCAAAAGTAAAGGCTTTAAATTATAACATATGGGATTCTTAGTAG
TATGTTTTTTTCTTGAAACTCAGTGGCTCTATCTAACCTTACTATCTCCTCACTCTTTCTCTAA
GACTAAACTCTAGGCTCTTAAAAATCTGCCCACACCAATCTTAGAAGCTCTGAAAAGAATTTGT
CTTTAAATATCTTTTAATAGTAACATGTATTTTATGGACCAAATTGACATTTTCGACTATTTTT
TCCAAAAAAGTCAGGTGAATTTCAGCACACTGAGTTGGGAATTTCTTATCCCAGAAGACCAACC
AATTTCATATTTATTTAAGATTGATTCCATACTCCGTTTTCAAGGAGAATCCCTGCAGTCTCCT
TAAAGGTAGAACAAATACTTTCTATTTTTTTTTCACCATTGTGGGATTGGACTTTAAGAGGTGA
CTCTAAAAAAACAGAGAACAAATATGTCTCAGTTGTATTAAGCACGGACCCATATTATCATATT
CACTTAAAAAAATGATTTCCTGTGCACCTTTTGGCAACTTCTCTTTTCAATGTAGGGAAAAACT
TAGTCACCCTGAAAACCCACAAAATAAATAAAACTTGTAGATGTGGGCAGAAGGTTTGGGGGTG
GACATTGTATGTGTTTAAATTAAACCCTGTATCACTGAGAAGCTGTTGTATGGGTCAGAGAAAA
TGAATGCTTAGAAGCTGTTCACATCTTCAAGAGCAGAAGCAAACCACATGTCTCAGCTATATTA
TTATTTATTTTTTATGCATAAAGTGAATCATTTCTTCTGTATTAATTTCCAAAGGGTTTTACCC
TCTATTTAAATGCTTTGAAAAACAGTGCATTGACAATGGGTTGATATTTTTCTTTAAAAGAAAA
ATATAATTATGAAAGCCAAGATAATCTGAAGCCTGTTTTATTTTAAAACTTTTTATGTTCTGTG
GTTGATGTTGTTTGTTTGTTTGTTTCTATTTTGTTGGTTTTTTACTTTGTTTTTTGTTTTGTTT
TGTTTTGTTTTGCATACTACATGCAGTTCTTTAACCAATGTCTGTTTGGCTAATGTAATTAAAG
TTGTTAATTTATATGAGTGCATTTCAACTATGTCAATGGTTTCTTAATATTTATTGTGTAGAAG
TACTGGTAATTTTTTTATTTACAATATGTTTAAAGAGATAACAGTTTGATATGTTTTCATGTGT
TTATAGCAGAAGTTATTTATTTCTATGGCATTCCAGCGGATATTTTGGTGTTTGCGAGGCATGC
AGTCAATATTTTGTACAGTTAGTGGACAGTATTCAGCAACGCCTGATAGCTTCTTTGGCCTTAT
GTTAAATAAAAAGACCTGTTTGGGATGTA.

In some embodiments, oligonucleotides may have a region of complementarity to a mouse INHBA sequence, for example, as provided by Gene ID: 16323; NCBI Ref. No: NM 008380.2:

(SEQ ID NO: 270)
GGGGTTCGCTAGTGGCTGCTCCTCCAGGCAGCACCGGGCCAGCGTGGAGTTGGAGCTTTGTGAA
GTAGCCAGTAAATCAGAACGCCTCCGCTAGGTGCAGAGCGCGGTGGCAGCGGGCCACTCTGCCA
GTGCGGTAGTCGGTGGGACCGAACTCTACACTCGGGAAGGGGCAGTCTGCGGGTGCGGGGCCTG
AGCTGCCGCTCGCCTCCGTTGGCCAGGAGACCGGCAGCCCCACTGCAGCTGCCAAAAGGGGGGG
AAAAATCAAGAGCTGCGCTTTTAAACGAAGTTGCCCTTGCTGGTGTTCAGGGTAAAAATAGAGG
CGGCCGCTTGGACCAGCTTGGCCCCTGAGTCCAGGCGTCCCGCGAGCCGGGCTGGAGCTGCGCA
TTCGGGAGTGATCCCTGGAAACTGCCAGCAGGTGCTGCTCAAGTGCCAATACCATGAAGAGGAA
TTCAGACAGCTCTGACCTCATGAGACAAGAGCCGGCTGACAAAACAGAAGGGACCCGAAAGAGA
ATTTGCTGAAGAGGAGAAGGAAAAAAAAAGTCCAAAAAAACCTGTGCGTGAGGGGTGGGGAGGA
AAAGCAGGGCCTTTAAAGAAGGCAACCACACGACTTTTGCTGCCAGGATGCCCTTGCTTTGGCT
GAGAGGATTTCTGTTGGCAAGTTGCTGGATTATAGTGAGGAGTTCCCCCACCCCAGGATCCGAG
GGGCACGGCTCAGCCCCGGACTGCCCGTCCTGTGCGCTGGCCACCCTTCCGAAGGATGGACCTA
ACTCTCAGCCAGAGATGGTAGAGGCTGTCAAGAAGCACATCTTAAACATGCTGCACTTGAAGAA
GAGACCCGATGTCACCCAGCCGGTGCCCAAGGCGGCGCTTCTCAACGCGATCAGAAAGCTTCAT
GTGGGTAAAGTGGGGGAGAACGGGTATGTGGAGATAGAGGACGACATTGGCAGGAGGGCCGAAA
TGAATGAACTCATGGAGCAGACCTCGGAGATCATCACCTTTGCCGAGTCAGGCACAGCCAGGAA
GACACTGCACTTTGAGATTTCCAAGGAAGGCAGTGACCTGTCAGTAGTGGAGCGTGCAGAAGTG
TGGCTCTTCCTGAAAGTCCCCAAGGCTAACAGAACCAGGACCAAAGTCACCATCCGTCTATTTC
AGCAGCAGAAGCACCCACAGGGCAGCTTGGACACGGGGGATGAGGCCGAGGAAATGGGCTTAAA
GGGGGAGAGGAGTGAACTGTTGCTATCAGAGAAAGTAGTTGATGCTCGGAAGAGTACCTGGCAC
ATCTTTCCAGTGTCCAGCAGCATCCAGCGCCTGCTGGACCAGGGAAAGAGTTCCCTGGACGTGC
GGATTGCTTGTGAGCAGTGCCAGGAGAGTGGTGCCAGTCTAGTGCTTCTGGGCAAGAAGAAGAA
GAAAGAGGTGGATGGAGATGGGAAGAAGAAAGATGGGAGTGACGGAGGGCTGGAAGAGGAAAAG
GAACAGTCACATAGACCTTTCCTCATGCTGCAGGCTAGGCAGTCCGAAGACCACCCTCATCGCA
GGCGTAGGCGGGGCTTGGAGTGCGACGGCAAGGTCAACATTTGCTGTAAGAAACAGTTCTTTGT
CAGCTTCAAGGACATTGGCTGGAATGACTGGATCATTGCTCCCTCTGGCTATCACGCCAATTAT
TGTGAGGGGGAGTGCCCAAGCCACATAGCAGGCACCTCTGGGTCCTCGCTCTCCTTCCACTCAA
CAGTCATTAACCACTACCGCATGAGGGGTCACAGCCCCTTTGCCAACCTTAAGTCATGCTGTGT
GCCCACCAAGCTGAGACCCATGTCCATGCTGTATTACGATGATGGTCAAAACATCATCAAAAAG
GACATTCAAAACATGATTGTGGAGGAGTGTGGCTGCTCCTAGAGTCGCCAGGTCCCAGAGAAAA
TGGATCTAGAGAGTCCAGAGAAGACAGTGGCAAAATGAAGAAAAAAATATAAGATTTATGAACT
AAACAAAACAACCAGAAAAATAGAAATAATAATAATAAAAAACCCACAAAAAAAAAACAAAAAC
AAAAATCAAAAACTAAACTGAAAACAAGACCTAATGAAACAGATGAAGGAAGATGTGGAAAAAT
ATCCTAAGGCAGGGCTCAGAGATGAAGCAGTAAAGGAGACAGGGATTGGGGGGGGGGAGGGGGG
AGAAGAGAGAATGGTGTACCTTCATTTCTTCCAAAACCAAACTGATTGCATCAGTTTTATCCAA
ACTGGGTATTGTCCTCTCTCCTGCCTCTTGCGGTTCCCTTGCGAGCCTGGAAGTCTACTTGTCT
ATTCTGCAGTAATGTGGGTTAGCACAACCCAAATAATAATGTCTAGAAAGCCATGAGTTTTAAA
GGGCCAGTCCCACCCACTTACCCAGGTTATAAGTATGTCTATGTGACACTATCTCTGTGTATTT
CAACACACACACACACACACACACACTCACACACACACACACACACACACACACACACACACAC
GCCCCCCCACACACACACACACTCACACACACACACACACACACACACACACACACACACACAC
TCACACACACACACACACACACACACGCCCACACACACAAACACAGAGGTGTTTCCACACACCA
CATGCATACACATACTGGTAAAAGAACAATTCTGTGCAGGTGGTCACATTTTCTTTTCTGTACC
ACTTTTGCCACCAGACAAAACCAACATAAAACATTGAGAACAAGAGTGGAAAGAATGAAAGACC
AAGGGAAGAAGAATACCAAGTTACATTTCGTTAAGGTGCTTTTGATCCTAGAACTATGCAACCT
AATAGGTTTGAAACTGTTTACCTGAAAGAGGACAAAAAGAGAGACTTTTTTGTATTGGAAGTAA
CCTGATTAATTTTTATTTTCTTCAAGGAGAGATACTTGAAAGGAATATGTTTGTCCATCTGTTG
GATTCAAACATTTCTATATTTTGTAAATGTTGTTTTTTTTATCGTTTACTATTTGCACTACGAT
GGTGTTTGACCTGTCTAATCCTTATTTAACAAGTATTTTCTTTGGGTGGGGGTGGGGGTGGGGT
TTAAGAGCTGCACTTCATGTGAGCTATAAAAGAACTGCTACAGCACACAAAATAGCTATTTTTA
TTATTATAATTATAATTATTATTATTATTTTGTACCTTAAAAATAGACACATACACCAAAGACA
TTTGTGTGAGCCTTTAAACAGTCTGTCTGTGGTTGGTATTGTTCACCATCAATGAGTCAGGGGT
ACAGATTTAAGGTTGAGTTAGGTAGATTGTGTTCAGGCTTAAAAGACCTGAGAGGTTTGGGTTT
TGACTCTTTTACATCCATGAAACAGGACATTTCATACTGGATGTACAGTAGTGTACACTGTTGG
ATTATCAAGTTCAAATTCATGAGACTACATGCTTGTATGTGTATATATACATTGCTTGTGCATA
TGCATATCTGTATGTATATATACATGTATTGTACCATGTCCATACACATTTTAAGCACTTCAGG
CTGTCATTTTAAAAATGTTCTTAAAACAATGAATGTTTGTGTGCAAAACACAGTATTTTTAAGA
AGGATAAGTGATAGATTTTTTTTTTTCTTGCTTTTACTCTGTAGTACGTGGGTACATTTCAAAT
GTTAGGATGGGGAAAGACTGAAAATCCCAGTGAGTATCCAGCCAGGCCCTCTTTAAATGTACAG
GATGAAATCCCCTCTTTCATATCCCCCCTGCTCCCTACAAGTTATCCCCTGTGGGGAAAAATGG
GATGTTACTTTAAAAACAAAATGGGCTGATTTTTTCAACTTATATTTATTATTTATTGGAATCA
ATCTTAAATCAGAAACATTTTTGGAAAAAATCTTTAGGCTAGAATAATTTTTTGAATAGTGTTA
TTACTACTTAAATAATAAAATAAGCAGGAAAGTATTTAAGACAGTGAGAGTTAAGGGAGAGAGC
ACTCAGGAGCCAGGGAGTTGTACAAATCTCTAATATTCTATTTTGCAGCCAAAAAACTTGCTGT
GTATGTTTGTACTTTTTCAGAGGCAAAACTGAAAAGATTGTCTTACGAATATCAAAATACACAC
TTAACCCAAGTTCCTAATTTAACCCAGTGTTGGCTTGTCTAAAACAGCTAATCAGTTATTCATT
TACATATTTAAAATATAGAGCCTTATTTTTACGGACTTGTTTGAAGTTTGAAAAACTTTATAAA
AGTGAAAAACTCTAATTGAAAAAAAATCTATATTCCTCAGTATTTTGTAAAATATTGAAGCTTT
TAGCATTAAGTCAGTCCATAGATTAAAGTCATTGTAGGAAAATGAAATACAAAGGAGAAATTAA
ATCTTAAAAAAGCTGGTTTAACTCTTAAAAAAATAAACTAACTCAATATGTATTAAATATACCG
TCAATATACCTTATCACATTAGGCTGTGTGTAGGCAAACTACTTTAGTCTTGTTACTGGGCAAA
CATATTTACTGTGTTCCAGGGGCCCTCCCTGTCTTATGCAGGAAATGCATGTACTGCATAAGAA
ATGAATTATAATTAAGGTCTAATGACCTTGAAGATCTCGCATGCGAGGACAGATGGCATGTTGA
GGACACAGAGGTGAGGTGGATGTCCAGGTTCAGCTTTGCCAATTTTTGTGAAAAACCTTTAGCG
CCCTCTGAACTGTTTCTTTAGTATTGGAGCTAATGCCGAGGCCTAGAGAAATAGTGGGCAAGAG
ATCTAACTGTGCCATATCAGAGATGATCTAGACCATGGGAAGAGCAGGATTTATGTAACTACTA
ATTATAGTTCTCATTCATCTGAGATGAATCTGCAATCTTCTTAAGCCCTTTAAATTCTAGATGT
TTTGAGGGTAAGCTTGGGTTTAATTAGTAGCCATAGTAATTAAATCTAGAAAGAAATGAAATTC
CACAGGACAGTGTATTTACTGGAGACCAAGTGACTTGGTTGTCACATAAACCTCATCAGAACTC
ATCAAATTTGTATGGCCTTGCAATAGAATTTAAATTGCTAATATTTTAACAATATTAGATATTG
TTAACAATTTAGAAAACATGAAGTCTTGTGAACTGGTCTTTCTACATAAGTGCTTAATCCAAAA
TTTGAAAAGCCTTTAATGCTTAAGATCTTAGTTTCTTCATGGTGTGCTTTCCCTAGTGTTAAAG
TGGCTCTGTCTGGTCTCCCCACTTTCTCTAGGATAATTCTTAAATACCTGCCCACACAAGTTCT
AGATGCTCTGAAGAGCATTTGTAGTTAGTATCTCTTTAATACTTGTAAGCTTCATTGACACTTT
TCCTTCCCAAAATAAGTCAAATTTCAGCACAGCAATGGGGATTTTCTTATCTTAGAAGACCAGC
CAATTCTATGTTCATTTAAGATTGATTCCACACTCCATTTTCAAGGAGAGGCCTTGTGTTTTCT
TAAAAGGCAGAATAAGTAAAATTGGGAGCTATGCCAGACTGAACGCAAGACGTGACTTTGTGAT
TCCAGAACAAACATGCCTCAGTTATAGTAACATGCATTCAAATGATTGTGTCACTTGAAAAATA
TGATTTCCTGTGGGCCTTTTGGCAACTTCTCTTTTTAGTATCGAGAAAAATGTAATCACCCCAA
AACCCGCATAAGTGTGACTTGTAGATGTGGGCAGGAGGTTGGGGGATGGACATTGTATGTGTTT
AAATTAAACCCTGTATCACTGAGAAGCTATTGGAGGGGTCAGAGATAATGAATGCATAGATGCT
GTTCACATCTTCAAGAGCAAAAGCAAATCACGTGTCTCAGCTATATTATTATTTATTTTTATGC
ATAAAGTGAATCATTTCTTCTGTATTAATTTTCAGTGGGGTTTGCCCTCTATTTAAATGCTTTG
AAAAACAGTGCATTGACAATGGTTGATATTTTTCTTTAAAAGAAAAATATAATTATGAAAGCCA
AGATAATCTGAAATCTGTTTTGATCTAAAACTTTTTATGTTATGTGGTTGATGTTGTTTGTTTG
TTTTTTATTTTTATTTTGTGAGTTCCTTTGCATACTACATGCAATTCTTTAACCAATGTCTGGC
TAATGTAATTAAAGTTGTTAATTTATATGAGTGCATTTCAACTATGTCAATGGTTTCTTAATAT
TTATTTTGTAGAAGTGCTGGTAATTTTTTATTTACGATATGTTTAAAGAGATAACGGTTGGATA
TGTTTTCATGTGTTTATAGCAGAAGTTATTTATTTCTATTCCATTCCAGCGGATATTCTGATGT
TTGCGAGGCATGCAGTCAATACTTTGTACAGTTAGTAGGCAGTATTCAGCAATGCCCGATAGCT
TCTTTGGCCTTATGTTAAATAAAAAGACCTGTTTGGGATGTAAAAAAAAAAAAAAAAAAAAAAA
AAA.

In some embodiments, an oligonucleotide comprises a region of complementarity to an INHBA sequence as set forth in SEQ ID NO: 269 or SEQ ID NO: 270. In some embodiments, the oligonucleotide comprises a region of complementarity that is at least 80%, 85%, 90%, 95%, 96%, 97%, 98%, 99%, or 100% complementary to an INHBA sequence as set forth in SEQ ID NO: 269 or SEQ ID NO: 270. In some embodiments, the oligonucleotide comprises a sequence that has at least 10, 11, 12, 13, 14, 15, 16, 17, 18, or 19 consecutive nucleotides that are perfectly complementary to an INHBA sequence as set forth in SEQ ID NO: 269 or SEQ ID NO: 270. In some embodiments, an oligonucleotide may comprise a sequence that targets (e.g., is complementary to) an RNA version (i.e., wherein the T's are replaced with U's) of an INHBA sequence as set forth in SEQ ID NO: 269 or SEQ ID NO: 270. In some embodiments, the oligonucleotide comprises a sequence that is complementary (e.g., at least 80%, 85%, 90%, 95%, 96%, 97%, 98%, 99%, or 100% complementary) to an RNA version of an INHBA sequence as set forth in SEQ ID NO: 269 or SEQ ID NO: 270. In some embodiments, the oligonucleotide comprises a sequence that has at least 10, 11, 12, 13, 14, 15, 16, 17, 18, or 19 consecutive nucleotides that are perfectly complementary to an RNA version of an INHBA sequence as set forth in SEQ ID NO: 269 or SEQ ID NO: 270.

In some embodiments, an INHBA-targeting oligonucleotide comprises an antisense strand that comprises at least 10, 11, 12, 13, 14, 15, 16, 17, 18, or 19 consecutive nucleotides of a sequence comprising any one of SEQ ID NOs: 319-342. In some embodiments, an INHBA-targeting oligonucleotide comprises an antisense strand that comprises any one of SEQ ID NO: 319-342. In some embodiments, an oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 319-342.

In some embodiments, an INHBA-targeting oligonucleotide comprises an antisense strand that targets an INHBA sequence comprising any one of SEQ ID NO: 271-318. In some embodiments, an oligonucleotide comprises an antisense strand comprising at least 10, 11, 12, 13, 14, 15, 16, 17, 18, or 19 nucleotides (e.g., consecutive nucleotides) that are complementary to an INHBA sequence comprising any one of SEQ ID NO: 271-318. In some embodiments, an INHBA-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NO: 271-318.

In some embodiments, an INHBA-targeting oligonucleotide comprises an antisense strand that comprises a region of complementarity to a target sequence as set forth in any one of SEQ ID NOs: 271-318. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, an INHBA-targeting oligonucleotide further comprises a sense strand that hybridizes to the antisense strand to form a double stranded siRNA. In some embodiments, the INHBA-targeting oligonucleotide comprises an antisense strand that comprises the nucleotide sequence of any one of SEQ ID NOs: 319-342. In some embodiments, the INHBA-targeting oligonucleotide further comprises a sense strand that comprises the nucleotide sequence of any one of SEQ ID NOs: 295-318.

In some embodiments, the INHBA-targeting oligonucleotide is a double stranded oligonucleotide (e.g., an siRNA) comprising an antisense strand that comprises the nucleotide sequence of any one of SEQ ID NOs: 319-342 and a sense strand that hybridizes to the antisense strand and comprises the nucleotide sequence of any one of SEQ ID NOs: 295-318, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the INHBA-targeting oligonucleotide is a double stranded oligonucleotide (e.g., an siRNA) comprising an antisense strand that comprises the nucleotide sequence of any one of SEQ ID NOs: 319-342 and a sense strand that hybridizes to the antisense strand and comprises the nucleotide sequence of any one of SEQ ID NOs: 295-318, wherein each nucleoside in the antisense strand and/or (e.g., and) each nucleoside in the sense strand is a 2′-modified nucleoside selected from 2′-O-Me and 2′-F modified nucleosides.

In some embodiments, the INHBA-targeting oligonucleotide is a double stranded oligonucleotide (e.g., an siRNA) comprising an antisense strand that comprises the nucleotide sequence of any one of SEQ ID NOs: 319-342 and a sense strand that hybridizes to the antisense strand and comprises the nucleotide sequence of any one of SEQ ID NOs: 295-318, wherein each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the INHBA-targeting oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates a phosphorothioate internucleoside linkage; and the absence of “*” between two nucleosides indicates a phosphodiester internucleoside linkage.

In some embodiments, the sense strand of the INHBA-targeting oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicates a phosphodiester internucleoside linkage.

In some embodiments, the antisense strand of the INHBA-targeting oligonucleotide is selected from the modified version of SEQ ID NOs: 319-342 listed in Table 13. In some embodiments, the sense strand of the INHBA-targeting oligonucleotide is selected from the modified version of SEQ ID NOs: 295-318 listed in Table 13. In some embodiments, the INHBA-targeting oligonucleotide is an siRNA selected from the siRNAs listed in Table 13.

TABLE 11
INHBA Target Sequences

| Corresponding | | |
|---|---|---|
| nucleotides of | INHBA | |
| Sequence NM_002192.4 | Target Sequence | SEQ |
| (SEQ ID NO: 422) | (5′ to 3′) | ID NO: |
| 227-245 | AGGATGCCCTTGCTTTGGC | 271 |
| 228-246 | GGATGCCCTTGCTTTGGCT | 272 |
| 237-255 | TGCTTTGGCTGAGAGGATT | 273 |
| 246-264 | TGAGAGGATTTCTGTTGGC | 274 |
| 250-268 | AGGATTTCTGTTGGCAAGT | 275 |
| 252-270 | GATTTCTGTTGGCAAGTTG | 276 |
| 260-278 | TTGGCAAGTTGCTGGATTA | 277 |
| 261-279 | TGGCAAGTTGCTGGATTAT | 278 |
| 262-280 | GGCAAGTTGCTGGATTATA | 279 |
| 264-282 | CAAGTTGCTGGATTATAGT | 280 |
| 267-285 | GTTGCTGGATTATAGTGAG | 281 |
| 272-290 | TGGATTATAGTGAGGAGTT | 282 |
| 273-291 | GGATTATAGTGAGGAGTTC | 283 |
| 489-507 | TCAGAAAGCTTCATGTGGG | 284 |
| 521-539 | AACGGGTATGTGGAGATAG | 285 |
| 522-540 | ACGGGTATGTGGAGATAGA | 286 |
| 523-541 | CGGGTATGTGGAGATAGAG | 287 |
| 525-543 | GGTATGTGGAGATAGAGGA | 288 |
| 582-600 | AGCAGACCTCGGAGATCAT | 289 |
| 728-746 | ACCAGGACCAAAGTCACCA | 290 |
| 1191-1209 | GCTGTAAGAAACAGTTCTT | 291 |
| 1231-1249 | CTGGAATGACTGGATCATT | 292 |
| 1328-1346 | TCCTTCCACTCAACAGTCA | 293 |
| 1407-1425 | CCACCAAGCTGAGACCCAT | 294 |

*The target sequences contain Ts, but binding to RNA and/or DNA is contemplated.

In some embodiments, an oligonucleotide may comprise or consist of any sequence as provided in Table 12.

TABLE 12
Oligonucleotide sequences for targeting INHBA

| Passenger Strand/ | | Guide Strand/ | |
|---|---|---|---|
| Sense Strand | SEQ ID | Antisense Strand | SEQ ID |
| (RNA) (5′ to 3′) | NO: | (RNA) (5′ to 3′) | NO: |
| CCAGGAUGCCCUUGCUUUGGC | 295 | GCCAAAGCAAGGGCAUCCUGGCA | 319 |
| CAGGAUGCCCUUGCUUUGGCU | 296 | AGCCAAAGCAAGGGCAUCCUGGC | 320 |
| CUUGCUUUGGCUGAGAGGAUU | 297 | AAUCCUCUCAGCCAAAGCAAGGG | 321 |
| GCUGAGAGGAUUUCUGUUGGC | 298 | GCCAACAGAAAUCCUCUCAGCCA | 322 |
| AGAGGAUUUCUGUUGGCAAGU | 299 | ACUUGCCAACAGAAAUCCUCUCA | 323 |
| AGGAUUUCUGUUGGCAAGUUG | 300 | CAACUUGCCAACAGAAAUCCUCU | 324 |
| UGUUGGCAAGUUGCUGGAUUA | 301 | UAAUCCAGCAACUUGCCAACAGA | 325 |
| GUUGGCAAGUUGCUGGAUUAU | 302 | AUAAUCCAGCAACUUGCCAACAG | 326 |
| UUGGCAAGUUGCUGGAUUAUA | 303 | UAUAAUCCAGCAACUUGCCAACA | 327 |
| GGCAAGUUGCUGGAUUAUAGU | 304 | ACUAUAAUCCAGCAACUUGCCAA | 328 |
| AAGUUGCUGGAUUAUAGUGAG | 305 | CUCACUAUAAUCCAGCAACUUGC | 329 |
| GCUGGAUUAUAGUGAGGAGUU | 306 | AACUCCUCACUAUAAUCCAGCAA | 330 |
| CUGGAUUAUAGUGAGGAGUUC | 307 | GAACUCCUCACUAUAAUCCAGCA | 331 |
| GAUCAGAAAGCUUCAUGUGGG | 308 | CCCACAUGAAGCUUUCUGAUCGC | 332 |
| AGAACGGGUAUGUGGAGAUAG | 309 | CUAUCUCCACAUACCCGUUCUCC | 333 |
| GAACGGGUAUGUGGAGAUAGA | 310 | UCUAUCUCCACAUACCCGUUCUC | 334 |
| AACGGGUAUGUGGAGAUAGAG | 311 | CUCUAUCUCCACAUACCCGUUCU | 335 |
| CGGGUAUGUGGAGAUAGAGGA | 312 | UCCUCUAUCUCCACAUACCCGUU | 336 |
| GGAGCAGACCUCGGAGAUCAU | 313 | AUGAUCUCCGAGGUCUGCUCCAU | 337 |
| GGACCAGGACCAAAGUCACCA | 314 | UGGUGACUUUGGUCCUGGUCCUG | 338 |
| CUGCUGUAAGAAACAGUUCUU | 315 | AAGAACUGUUUCUUACAGCAGAU | 339 |
| GGCUGGAAUGACUGGAUCAUU | 316 | AAUGAUCCAGUCAUUCCAGCCGA | 340 |
| UGUCCUUCCACUCAACAGUCA | 317 | UGACUGUUGAGUGGAAGGACAGU | 341 |
| GCCCACCAAGCUGAGACCCAU | 318 | AUGGGUCUCAGCUUGGUGGGCAC | 342 |

In some embodiments, an oligonucleotide is a modified oligonucleotide as provided in Table 13, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 13
Modified Oligonucleotides for targeting INHBA

| | Modified Passenger | | Modified Guide | |
|---|---|---|---|---|
| | Strand/Sense Strand | SEQ ID | Strand/Antisense Strand | SEQ ID |
| siRNA # | (RNA) (5′ to 3′) | NO: | (RNA) (5′ to 3′) | NO: |
| hsINHBA-4 | mCmCfAmGfGmAfUmGfC | 295 | fGfCmCfAmAfAmGfCmAfAmGf | 319 |
| | mCfCmUfUmGfCmUfUmUf | | GmGfCmAfUmCfCmUfGmG*fC | |
| | GmGfC | | *mA | |
| hsINHBA-5 | mCmAfGmGfAmUfGmCfC | 296 | fAfGmCfCmAfAmAfGmCfAmAf | 320 |
| | mCfUmUfGmCfUmUfUmGf | | GmGfGmCfAmUfCmCfUmG*fG | |
| | GmCfU | | *mC | |
| hsINHBA-6 | mCmUfUmGfCmUfUmUfG | 297 | fAfAmUfCmCfUmCfUmCfAmGf | 321 |
| | mGfCmUfGmAfGmAfGmG | | CmCfAmAfAmGfCmAfAmG*fG | |
| | fAmUfU | | *mG | |
| hsINHBA-7 | mGmCfUmGfAmGfAmGfG | 298 | fGfCmCfAmAfCmAfGmAfAmAf | 322 |
| | mAfUmUfUmCfUmGfUmU | | UmCfCmUfCmUfCmAfGmC*fC* | |
| | fGmGfC | | mA | |
| hsINHBA-8 | mAmGfAmGfGmAfUmUfU | 299 | fAfCmUfUmGfCmCfAmAfCmAf | 323 |
| | mCfUmGfUmUfGmGfCmAf | | GmAfAmAfUmCfCmUfCmU*fC | |
| | AmGfU | | *mA | |
| hsINHBA-9 | mAmGfGmAfUmUfUmCfU | 300 | fCfAmAfCmUfUmGfCmCfAmAf | 324 |
| | mGfUmUfGmGfCmAfAmG | | CmAfGmAfAmAfUmCfCmU*fC | |
| | fUmUfG | | *mU | |
| hsINHBA-10 | mUmGfUmUfGmGfCmAfA | 301 | fUfAmAfUmCfCmAfGmCfAmAf | 325 |
| | mGfUmUfGmCfUmGfGmA | | CmUfUmGfCmCfAmAfCmA*fG | |
| | fUmUfA | | *mA | |
| hsINHBA-11 | mGmUfUmGfGmCfAmAfG | 302 | fAfUmAfAmUfCmCfAmGfCmAf | 326 |
| | mUfUmGfCmUfGmGfAmU | | AmCfUmUfGmCfCmAfAmC*fA | |
| | fUmAfU | | *mG | |
| hsINHBA-12 | mUmUfGmGfCmAfAmGfU | 303 | fUfAmUfAmAfUmCfCmAfGmCf | 327 |
| | mUfGmCfUmGfGmAfUmU | | AmAfCmUfUmGfCmCfAmA*fC | |
| | fAmUfA | | *mA | |
| hsINHBA-1 | mGmGfCmAfAmGfUmUfG | 304 | fAfCmUfAmUfAmAfUmCfCmAf | 328 |
| | mCfUmGfGmAfUmUfAmU | | GmCfAmAfCmUfUmGfCmC*fA | |
| | fAmGfU | | *mA | |
| hsINHBA-2 | mAmAfGmUfUmGfCmUfG | 305 | fCfUmCfAmCfUmAfUmAfAmUf | 329 |
| | mGfAmUfUmAfUmAfGmU | | CmCfAmGfCmAfAmCfUmU*fG | |
| | fGmAfG | | *mC | |
| hsINHBA-13 | mGmCfUmGfGmAfUmUfA | 306 | fAfAmCfUmCfCmUfCmAfCmUf | 330 |
| | mUfAmGfUmGfAmGfGmA | | AmUfAmAfUmCfCmAfGmC*fA | |
| | fGmUfU | | *mA | |
| hsINHBA-14 | mCmUfGmGfAmUfUmAfU | 307 | fGfAmAfCmUfCmCfUmCfAmCf | 331 |
| | mAfGmUfGmAfGmGfAmG | | UmAfUmAfAmUfCmCfAmG*fC | |
| | fUmUfC | | *mA | |
| hsINHBA-15 | mGmAfUmCfAmGfAmAfA | 308 | fCfCmCfAmCfAmUfGmAfAmGf | 332 |
| | mGfCmUfUmCfAmUfGmUf | | CmUfUmUfCmUfGmAfUmC*fG | |
| | GmGfG | | kmC | |
| hsINHBA-16 | mAmGfAmAfCmGfGmGfU | 309 | fCfUmAfUmCfUmCfCmAfCmAf | 333 |
| | mAfUmGfUmGfGmAfGmA | | UmAfCmCfCmGfUmUfCmU*fC | |
| | fUmAfG | | *mC | |
| hsINHBA-17 | mGmAfAmCfGmGfGmUfA | 310 | fUfCmUfAmUfCmUfCmCfAmCf | 334 |
| | mUfGmUfGmGfAmGfAmU | | AmUfAmCfCmCfGmUfUmC*fU | |
| | fAmGfA | | *mC | |
| hsINHBA-18 | mAmAfCmGfGmGfUmAfU | 311 | fCfUmCfUmAfUmCfUmCfCmAf | 335 |
| | mGfUmGfGmAfGmAfUmA | | CmAfUmAfCmCfCmGfUmU*fC | |
| | fGmAfG | | *mU | |
| hsINHBA-19 | mCmGfGmGfUmAfUmGfU | 312 | fUfCmCfUmCfUmAfUmCfUmCf | 336 |
| | mGfGmAfGmAfUmAfGmA | | CmAfCmAfUmAfCmCfCmG*fU | |
| | fGmGfA | | *mU | |
| hsINHBA-20 | mGmGfAmGfCmAfGmAfC | 313 | fAfUmGfAmUfCmUfCmCfGmAf | 337 |
| | mCfUmCfGmGfAmGfAmUf | | GmGfUmCfUmGfCmUfCmC*fA | |
| | CmAfU | | *mU | |
| hsINHBA-21 | mGmGfAmCfCmAfGmGfA | 314 | fUfGmGfUmGfAmCfUmUfUmGf | 338 |
| | mCfCmAfAmAfGmUfCmAf | | GmUfCmCfUmGfGmUfCmC*fU | |
| | CmCfA | | *mG | |
| hsINHBA-3 | mCmUfGmCfUmGfUmAfA | 315 | fAfAmGfAmAfCmUfGmUfUmUf | 339 |
| | mGfAmAfAmCfAmGfUmU | | CmUfUmAfCmAfGmCfAmG*fA | |
| | fCmUfU | | *mU | |
| hsINHBA-22 | mGmGfCmUfGmGfAmAfU | 316 | fAfAmUfGmAfUmCfCmAfGmUf | 340 |
| | mGfAmCfUmGfGmAfUmCf | | CmAfUmUfCmCfAmGfCmC*fG | |
| | AmUfU | | *mA | |
| hsINHBA-23 | mUmGfUmCfCmUfUmCfC | 317 | fUfGmAfCmUfGmUfUmGfAmGf | 341 |
| | mAfCmUfCmAfAmCfAmGf | | UmGfGmAfAmGfGmAfCmA*fG | |
| | UmCfA | | *mU | |
| hsINHBA-24 | mGmCfCmCfAmCfCmAfA | 318 | fAfUmGfGmGfUmCfUmCfAmGf | 342 |
| | mGfCmUfGmAfGmAfCmCf | | CmUfUmGfGmUfGmGfGmC*fA | |
| | CmAfU | | *mC | |

c. ACVR1B Oligonucleotides

In some embodiments, the oligonucleotide is an antisense oligonucleotide (ASO). In some embodiments, the oligonucleotide is a siRNA. In some embodiments, the oligonucleotide is a short hairpin RNA. In some embodiments, the oligonucleotide is a miRNA-based shRNA (e.g., a shRNA based on miR-24, miR-210, miR-199a-5p). In some embodiments, the oligonucleotide is a CRISPR guide RNA targeting ACVR1B. Examples of oligonucleotides useful for targeting ACVR1B are provided in Katoh M., “Cardio-miRNAs and onco-miRNAs: circulating miRNA-based diagnostics for non-cancerous and cancerous diseases.” Front Cell Dev Biol. 2014 Oct. 16; 2:61.; Mizuno, Y. et al. “miR-210 promotes osteoblastic differentiation through inhibition of AcvR1b.” FEBS Lett. 2009 Jul. 7; 583(13):2263-8.; Lin, H. S. et al., “miR-199a-5p inhibits monocyte/macrophage differentiation by targeting the activin A type 1B receptor gene and finally reducing C/EBPα expression.” J Leukoc Biol. 2014 Dec; 96(6):1023-35.; International Patent Application Publication WO 2016/161477, entitled “A method of treating neoplasias”, filed on Mar. 23, 2016; and U.S. Patent Application Publication US 2014/0088174, entitled “Compounds and methods for altering activin receptor-like kinase signaling”, published on Mar. 27, 2014; the contents of each of which are incorporated herein in their entireties.

In some embodiments, oligonucleotides may have a region of complementarity to a human ACVR1B sequence, for example, as provided below (Gene ID: 91; NCBI Ref. No: NM_004302.5):

(SEQ ID NO: 367)
GGGCGCTGCTGGGCTGCGGCGGCGGCGGCGGCGGCGGTGGTTACTATGGCGGAGTCGGCCGGAG
CCTCCTCCTTCTTCCCCCTTGTTGTCCTCCTGCTCGCCGGCAGCGGCGGGTCCGGGCCCCGGGG
GGTCCAGGCTCTGCTGTGTGCGTGCACCAGCTGCCTCCAGGCCAACTACACGTGTGAGACAGAT
GGGGCCTGCATGGTTTCCATTTTCAATCTGGATGGGATGGAGCACCATGTGCGCACCTGCATCC
CCAAAGTGGAGCTGGTCCCTGCCGGGAAGCCCTTCTACTGCCTGAGCTCGGAGGACCTGCGCAA
CACCCACTGCTGCTACACTGACTACTGCAACAGGATCGACTTGAGGGTGCCCAGTGGTCACCTC
AAGGAGCCTGAGCACCCGTCCATGTGGGGCCCGGTGGAGCTGGTAGGCATCATCGCCGGCCCGG
TGTTCCTCCTGTTCCTCATCATCATCATTGTTTTCCTTGTCATTAACTATCATCAGCGTGTCTA
TCACAACCGCCAGAGACTGGACATGGAAGATCCCTCATGTGAGATGTGTCTCTCCAAAGACAAG
ACGCTCCAGGATCTTGTCTACGATCTCTCCACCTCAGGGTCTGGCTCAGGGTTACCCCTCTTTG
TCCAGCGCACAGTGGCCCGAACCATCGTTTTACAAGAGATTATTGGCAAGGGTCGGTTTGGGGA
AGTATGGCGGGGCCGCTGGAGGGGTGGTGATGTGGCTGTGAAAATATTCTCTTCTCGTGAAGAA
CGGTCTTGGTTCAGGGAAGCAGAGATATACCAGACGGTCATGCTGCGCCATGAAAACATCCTTG
GATTTATTGCTGCTGACAATAAAGATAATGGCACCTGGACACAGCTGTGGCTTGTTTCTGACTA
TCATGAGCACGGGTCCCTGTTTGATTATCTGAACCGGTACACAGTGACAATTGAGGGGATGATT
AAGCTGGCCTTGTCTGCTGCTAGTGGGCTGGCACACCTGCACATGGAGATCGTGGGCACCCAAG
GGAAGCCTGGAATTGCTCATCGAGACTTAAAGTCAAAGAACATTCTGGTGAAGAAAAATGGCAT
GTGTGCCATAGCAGACCTGGGCCTGGCTGTCCGTCATGATGCAGTCACTGACACCATTGACATT
GCCCCGAATCAGAGGGTGGGGACCAAACGATACATGGCCCCTGAAGTACTTGATGAAACCATTA
ATATGAAACACTTTGACTCCTTTAAATGTGCTGATATTTATGCCCTCGGGCTTGTATATTGGGA
GATTGCTCGAAGATGCAATTCTGGAGGAGTCCATGAAGAATATCAGCTGCCATATTACGACTTA
GTGCCCTCTGACCCTTCCATTGAGGAAATGCGAAAGGTTGTATGTGATCAGAAGCTGCGTCCCA
ACATCCCCAACTGGTGGCAGAGTTATGAGGCACTGCGGGTGATGGGGAAGATGATGCGAGAGTG
TTGGTATGCCAACGGCGCAGCCCGCCTGACGGCCCTGCGCATCAAGAAGACCCTCTCCCAGCTC
AGCGTGCAGGAAGACGTGAAGATCTAACTGCTCCCTCTCTCCACACGGAGCTCCTGGCAGCGAG
AACTACGCACAGCTGCCGCGTTGAGCGTACGATGGAGGCCTACCTCTCGTTTCTGCCCAGCCCT
CTGTGGCCAGGAGCCCTGGCCCGCAAGAGGGACAGAGCCCGGGAGAGACTCGCTCACTCCCATG
TTGGGTTTGAGACAGACACCTTTTCTATTTACCTCCTAATGGCATGGAGACTCTGAGAGCGAAT
TGTGTGGAGAACTCAGTGCCACACCTCGAACTGGTTGTAGTGGGAAGTCCCGCGAAACCCGGTG
CATCTGGCACGTGGCCAGGAGCCATGACAGGGGCGCTTGGGAGGGGCCGGAGGAACCGAGGTGT
TGCCAGTGCTAAGCTGCCCTGAGGGTTTCCTTCGGGGACCAGCCCACAGCACACCAAGGTGGCC
CGGAAGAACCAGAAGTGCAGCCCCTCTCACAGGCAGCTCTGAGCCGCGCTTTCCCCTCCTCCCT
GGGATGGACGCTGCCGGGAGACTGCCAGTGGAGACGGAATCTGCCGCTTTGTCTGTCCAGCCGT
GTGTGCATGTGCCGAGGTGCGTCCCCCGTTGTGCCTGGTTCGTGCCATGCCCTTACACGTGCGT
GTGAGTGTGTGTGTGTGTCTGTAGGTGCGCACTTACCTGCTTGAGCTTTCTGTGCATGTGCAGG
TCGGGGGTGTGGTCGTCATGCTGTCCGTGCTTGCTGGTGCCTCTTTTCAGTAGTGAGCAGCATC
TAGTTTCCCTGGTGCCCTTCCCTGGAGGTCTCTCCCTCCCCCAGAGCCCCTCATGCCACAGTGG
TACTCTGTGTCTGGCAGGCTACTCTGCCCACCCCAGCATCAGCACAGCTCTCCTCCTCCATCTC
AGACTGTGGAACCAAAGCTGGCCCAGTTGTCCATGACAAAAGAGGCTTTTGGGCCAAAATGTGA
GGGTGGTGGGTGGGATGGGCAGGGAAGGAATCCTGGTGGAAGTCTTGGGTGTTAGTGTCAGCCA
TGGGAAATGAGCCAGCCCAAGGGCATCATCCTCAGCAGCATCGAGGAAGGGCCGAGGAATGTGA
AGCCAGATCTCGGGACTCAGATTGGAATGTTACATCTGTCTTTCATCTCCCAGATCCTGGAAAC
AGCAGTGTATATTTTTGGTGGTGGTGGGTTTGGGGTGGGGAAGGGAAGGGCGGGCAAGGAGTGG
GGAGGGAGTCTGGGGTGGGAGGGAGGCATCTGCATGGGTCTTCTTTTACTGGACTGTCTGATCA
GGGTGGAGGGAAGGTGAGAGGTTTGCATCCACTTCAGGAGCCCTACTGAAGGGAACAGCCTGAG
CCGAACATGTTATTTAACCTGAGTATAGTATTTAACGAAGCCTAGAAGCACGGCTGTGGGTGGT
GATTTGGTCAGCATATCTTAGGTATATAATAACTTTGAAGCCATAACTTTTAACTGGAGTGGTT
TGATTTCTTTTTTTAATTTTATTGGGAGGGTTTGGATTTTAACTTTTTTTAATGTTGTTAAATA
TTAAGTTTTTGTAAAAGGAAAACCATCTCTGTGATTACCTCTCAATCTATTTGTTTTTAAAGAA
ATCCCTAAAAAAAAAAATTATCCAATTGAACGCACATAGCTCAATCACACTGGAAATGTTTGTC
CTTGCACCTGAGCCTGTTCCCACTCAGCAGTGAGAGTTCCTCTTTGCCCTGAGGCTCAGTCTCT
CTCGTATTTTGTCCCCACCCCCAATTCCTTGAGTGGTTTTTGCTCTAGGGCCCTTTCTTGCACT
GTCCAGCTGGTTGTACCCTCTCCAGGCATTTATTCAACAAATGTGGGTGAAGTGCCTGCTGGGT
GCCAGGTGCTGGGAATACATCTGTGGACAAGACATGCTTGGGTCCTACTCCTGGAGCACTGTAA
AAAGAGCTGATTCAAGTAAGTAGATGCCTGTTTTGAGACCAGAAGGTTTCATAATTGGTTCTAC
GACCCTTTTGAGCCTAGAATTATTGTTCTTATATAAGATCACTGAAGAAAGAGGAACCCCCACA
ACCCCCTCCACAAAGAGACCAGGGGGGGGTGATGAGACCTGGGGTTTAGAACCCCAGGTGAGAC
CTCAAATCACTGCATTCATTCTGAGCCCCCTTCCTGTCCCCAGGGGAGGTGTATTGTGTATGTA
GCCTTAGAGCATCTCTGCCTCCAACCCAGCAGTTCTCTGCCAAAGCTTGTGGAGGAGGGAGAGC
CCTGTCCCTGCCCTCAGGCTCCCCAGTGCTCCTGGCCCTTCTATTTATTTGACTGATTATTGCT
TCTTTCCTTGCATTAAAGGAGATCTTCCCCTAACCTTTGGGCCAATTTACTGGCCACTAATTTC
GTTTAAATACCATTGTGTCATTGGGGGGACCGTCTTTACCCCTGCTGACCTCCCACCTATCCGC
CCTGCAGCAGAACCTTGGCGGTTTATAGGTAATGATGGAACTTAGACTCCTCTTCCCAGAGTCA
CAAGTAGCCTCTGGGATCTGCCAACACACGTCCACTCCCAAGCCACTAGCCCACTCCCCAGTTG
GCCCTTCTGCCCTTACCCCACACACAGTCCAACTCTTCCACCTCTGGGGAAGATGGAGCAGGTC
TTTGGGAAGCTCCCACACCCACCTCTGCCACTCTTAACACTAAGTGAGAGTTGGGGAGAAACTG
AAGCCGTGTTTTTGGCCCCCCGAGGCTAACCCTGATCCATAGTGCTACCTGCACCTCTGGATTC
TGGATTCACAGACCAAGTCCAAGCCCGTTCTTACGTCGCCATAAAGGCCCCCGAACGGCATTCT
CGGTACTTCTGTTTGTTTTTGTACATTTTATTAGAAAGGACTGTAAAATAGCCACTTAGACACT
TTACCTCTTCAGTATGCAAATGTAAATAAATTGTAATATAGGAAATCTTTTGTTTTAATATAAG
AATGAGCCTGTCCAATTTCTGCTGTACATTATTAAAAGTTTTATTCACAGA.

In some embodiments, oligonucleotides may have a region of complementarity to a human ACVR1B sequence, for example, as provided below (Gene ID: 91; NCBI Ref. No: NM_020328.4):

(SEQ ID NO: 368)
GGGCGCTGCTGGGCTGCGGCGGCGGCGGCGGCGGCGGTGGTTACTATGGC
GGAGTCGGCCGGAGCCTCCTCCTTCTTCCCCCTTGTTGTCCTCCTGCTCG
CCGGCAGCGGCGGGTCCGGGCCCCGGGGGGTCCAGGCTCTGCTGTGTGCG
TGCACCAGCTGCCTCCAGGCCAACTACACGTGTGAGACAGATGGGGCCTG
CATGGTTTCCATTTTCAATCTGGATGGGATGGAGCACCATGTGCGCACCT
GCATCCCCAAAGTGGAGCTGGTCCCTGCCGGGAAGCCCTTCTACTGCCTG
AGCTCGGAGGACCTGCGCAACACCCACTGCTGCTACACTGACTACTGCAA
CAGGATCGACTTGAGGGTGCCCAGTGGTCACCTCAAGGAGCCTGAGCACC
CGTCCATGTGGGGCCCGGTGGAGCTGGTAGGCATCATCGCCGGCCCGGTG
TTCCTCCTGTTCCTCATCATCATCATTGTTTTCCTTGTCATTAACTATCA
TCAGCGTGTCTATCACAACCGCCAGAGACTGGACATGGAAGATCCCTCAT
GTGAGATGTGTCTCTCCAAAGACAAGACGCTCCAGGATCTTGTCTACGAT
CTCTCCACCTCAGGGTCTGGCTCAGGGTTACCCCTCTTTGTCCAGCGCAC
AGTGGCCCGAACCATCGTTTTACAAGAGATTATTGGCAAGGGTCGGTTTG
GGGAAGTATGGCGGGGCCGCTGGAGGGGTGGTGATGTGGCTGTGAAAATA
TTCTCTTCTCGTGAAGAACGGTCTTGGTTCAGGGAAGCAGAGATATACCA
GACGGTCATGCTGCGCCATGAAAACATCCTTGGATTTATTGCTGCTGACA
ATAAAGCAGACTGCTCATTCCTCACATTGCCATGGGAAGTTGTAATGGTC
TCTGCTGCCCCCAAGCTGAGGAGCCTTAGACTCCAATACAAGGGAGGAAG
GGGAAGAGCAAGATTTTTATTCCCACTGAATAATGGCACCTGGACACAGC
TGTGGCTTGTTTCTGACTATCATGAGCACGGGTCCCTGTTTGATTATCTG
AACCGGTACACAGTGACAATTGAGGGGATGATTAAGCTGGCCTTGTCTGC
TGCTAGTGGGCTGGCACACCTGCACATGGAGATCGTGGGCACCCAAGGGA
AGCCTGGAATTGCTCATCGAGACTTAAAGTCAAAGAACATTCTGGTGAAG
AAAAATGGCATGTGTGCCATAGCAGACCTGGGCCTGGCTGTCCGTCATGA
TGCAGTCACTGACACCATTGACATTGCCCCGAATCAGAGGGTGGGGACCA
AACGATACATGGCCCCTGAAGTACTTGATGAAACCATTAATATGAAACAC
TTTGACTCCTTTAAATGTGCTGATATTTATGCCCTCGGGCTTGTATATTG
GGAGATTGCTCGAAGATGCAATTCTGGAGGAGTCCATGAAGAATATCAGC
TGCCATATTACGACTTAGTGCCCTCTGACCCTTCCATTGAGGAAATGCGA
AAGGTTGTATGTGATCAGAAGCTGCGTCCCAACATCCCCAACTGGTGGCA
GAGTTATGAGGCACTGCGGGTGATGGGGAAGATGATGCGAGAGTGTTGGT
ATGCCAACGGCGCAGCCCGCCTGACGGCCCTGCGCATCAAGAAGACCCTC
TCCCAGCTCAGCGTGCAGGAAGACGTGAAGATCTAACTGCTCCCTCTCTC
CACACGGAGCTCCTGGCAGCGAGAACTACGCACAGCTGCCGCGTTGAGCG
TACGATGGAGGCCTACCTCTCGTTTCTGCCCAGCCCTCTGTGGCCAGGAG
CCCTGGCCCGCAAGAGGGACAGAGCCCGGGAGAGACTCGCTCACTCCCAT
GTTGGGTTTGAGACAGACACCTTTTCTATTTACCTCCTAATGGCATGGAG
ACTCTGAGAGCGAATTGTGTGGAGAACTCAGTGCCACACCTCGAACTGGT
TGTAGTGGGAAGTCCCGCGAAACCCGGTGCATCTGGCACGTGGCCAGGAG
CCATGACAGGGGCGCTTGGGAGGGGCCGGAGGAACCGAGGTGTTGCCAGT
GCTAAGCTGCCCTGAGGGTTTCCTTCGGGGACCAGCCCACAGCACACCAA
GGTGGCCCGGAAGAACCAGAAGTGCAGCCCCTCTCACAGGCAGCTCTGAG
CCGCGCTTTCCCCTCCTCCCTGGGATGGACGCTGCCGGGAGACTGCCAGT
GGAGACGGAATCTGCCGCTTTGTCTGTCCAGCCGTGTGTGCATGTGCCGA
GGTGCGTCCCCCGTTGTGCCTGGTTCGTGCCATGCCCTTACACGTGCGTG
TGAGTGTGTGTGTGTGTCTGTAGGTGCGCACTTACCTGCTTGAGCTTTCT
GTGCATGTGCAGGTCGGGGGTGTGGTCGTCATGCTGTCCGTGCTTGCTGG
TGCCTCTTTTCAGTAGTGAGCAGCATCTAGTTTCCCTGGTGCCCTTCCCT
GGAGGTCTCTCCCTCCCCCAGAGCCCCTCATGCCACAGTGGTACTCTGTG
TCTGGCAGGCTACTCTGCCCACCCCAGCATCAGCACAGCTCTCCTCCTCC
ATCTCAGACTGTGGAACCAAAGCTGGCCCAGTTGTCCATGACAAAAGAGG
CTTTTGGGCCAAAATGTGAGGGTGGTGGGTGGGATGGGCAGGGAAGGAAT
CCTGGTGGAAGTCTTGGGTGTTAGTGTCAGCCATGGGAAATGAGCCAGCC
CAAGGGCATCATCCTCAGCAGCATCGAGGAAGGGCCGAGGAATGTGAAGC
CAGATCTCGGGACTCAGATTGGAATGTTACATCTGTCTTTCATCTCCCAG
ATCCTGGAAACAGCAGTGTATATTTTTGGTGGTGGTGGGTTTGGGGTGGG
GAAGGGAAGGGCGGGCAAGGAGTGGGGAGGGAGTCTGGGGTGGGAGGGAG
GCATCTGCATGGGTCTTCTTTTACTGGACTGTCTGATCAGGGTGGAGGGA
AGGTGAGAGGTTTGCATCCACTTCAGGAGCCCTACTGAAGGGAACAGCCT
GAGCCGAACATGTTATTTAACCTGAGTATAGTATTTAACGAAGCCTAGAA
GCACGGCTGTGGGTGGTGATTTGGTCAGCATATCTTAGGTATATAATAAC
TTTGAAGCCATAACTTTTAACTGGAGTGGTTTGATTTCTTTTTTTAATTT
TATTGGGAGGGTTTGGATTTTAACTTTTTTTAATGTTGTTAAATATTAAG
TTTTTGTAAAAGGAAAACCATCTCTGTGATTACCTCTCAATCTATTTGTT
TTTAAAGAAATCCCTAAAAAAAAAAATTATCCAATTGAACGCACATAGCT
CAATCACACTGGAAATGTTTGTCCTTGCACCTGAGCCTGTTCCCACTCAG
CAGTGAGAGTTCCTCTTTGCCCTGAGGCTCAGTCTCTCTCGTATTTTGTC
CCCACCCCCAATTCCTTGAGTGGTTTTTGCTCTAGGGCCCTTTCTTGCAC
TGTCCAGCTGGTTGTACCCTCTCCAGGCATTTATTCAACAAATGTGGGTG
AAGTGCCTGCTGGGTGCCAGGTGCTGGGAATACATCTGTGGACAAGACAT
GCTTGGGTCCTACTCCTGGAGCACTGTAAAAAGAGCTGATTCAAGTAAGT
AGATGCCTGTTTTGAGACCAGAAGGTTTCATAATTGGTTCTACGACCCTT
TTGAGCCTAGAATTATTGTTCTTATATAAGATCACTGAAGAAAGAGGAAC
CCCCACAACCCCCTCCACAAAGAGACCAGGGGGGGGTGATGAGACCTGGG
GTTTAGAACCCCAGGTGAGACCTCAAATCACTGCATTCATTCTGAGCCCC
CTTCCTGTCCCCAGGGGAGGTGTATTGTGTATGTAGCCTTAGAGCATCTC
TGCCTCCAACCCAGCAGTTCTCTGCCAAAGCTTGTGGAGGAGGGAGAGCC
CTGTCCCTGCCCTCAGGCTCCCCAGTGCTCCTGGCCCTTCTATTTATTTG
ACTGATTATTGCTTCTTTCCTTGCATTAAAGGAGATCTTCCCCTAACCTT
TGGGCCAATTTACTGGCCACTAATTTCGTTTAAATACCATTGTGTCATTG
GGGGGACCGTCTTTACCCCTGCTGACCTCCCACCTATCCGCCCTGCAGCA
GAACCTTGGCGGTTTATAGGTAATGATGGAACTTAGACTCCTCTTCCCAG
AGTCACAAGTAGCCTCTGGGATCTGCCAACACACGTCCACTCCCAAGCCA
CTAGCCCACTCCCCAGTTGGCCCTTCTGCCCTTACCCCACACACAGTCCA
ACTCTTCCACCTCTGGGGAAGATGGAGCAGGTCTTTGGGAAGCTCCCACA
CCCACCTCTGCCACTCTTAACACTAAGTGAGAGTTGGGGAGAAACTGAAG
CCGTGTTTTTGGCCCCCCGAGGCTAACCCTGATCCATAGTGCTACCTGCA
CCTCTGGATTCTGGATTCACAGACCAAGTCCAAGCCCGTTCTTACGTCGC
CATAAAGGCCCCCGAACGGCATTCTCGGTACTTCTGTTTGTTTTTGTACA
TTTTATTAGAAAGGACTGTAAAATAGCCACTTAGACACTTTACCTCTTCA
GTATGCAAATGTAAATAAATTGTAATATAGGAAATCTTTTGTTTTAATAT
AAGAATGAGCCTGTCCAATTTCTGCTGTACATTATTAAAAGTTTTATTCA
CAGA.

In some embodiments, oligonucleotides may have a region of complementarity to a mouse ACVR1 sequence, for example, as provided below (Gene ID: 11479; NCBI Ref. No: NM_007395.4)

(SEQ ID NO: 369)
GAGGGAGGGAGGGAGAGAGGCGCCGGGGGCGCGCGCGCGCGCTGGGCGCT
GCTGGGCTGCGGCGGCGGTTACTATGGCGGAGTCGGCCGGAGCCTCCTCC
TTCTTCCCCCTTGTTGTCCTCCTGCTCGCCGGCAGCGGCGGGTCCGGGCC
CCGGGGGATCCAGGCTCTGCTGTGTGCGTGCACCAGCTGCCTACAGACCA
ACTACACCTGTGAGACAGATGGGGCTTGCATGGTCTCCATCTTTAACCTG
GATGGCGTGGAGCACCATGTACGTACCTGCATCCCCAAGGTGGAGCTGGT
TCCTGCTGGAAAGCCCTTCTACTGCCTGAGTTCAGAGGATCTGCGCAACA
CACACTGCTGCTATATTGACTTCTGCAACAAGATTGACCTCAGGGTCCCC
AGCGGACACCTCAAGGAGCCTGCGCACCCCTCCATGTGGGGCCCTGTGGA
GCTGGTCGGCATCATCGCCGGCCCCGTCTTCCTCCTCTTCCTTATCATTA
TCATCGTCTTCCTGGTCATCAACTATCACCAGCGTGTCTACCATAACCGC
CAGAGGTTGGACATGGAGGACCCCTCTTGCGAGATGTGTCTCTCCAAAGA
CAAGACGCTCCAGGATCTCGTCTACGACCTCTCCACGTCAGGGTCTGGCT
CAGGGTTACCCCTTTTTGTCCAGCGCACAGTGGCCCGAACCATTGTTTTA
CAAGAGATTATCGGCAAGGGCCGGTTCGGGGAAGTATGGCGTGGTCGCTG
GAGGGGTGGTGACGTGGCTGTGAAAATCTTCTCTTCTCGTGAAGAACGGT
CTTGGTTCCGTGAAGCAGAGATCTACCAGACCGTCATGCTGCGCCATGAA
AACATCCTTGGCTTTATTGCTGCTGACAATAAAGATAATGGCACCTGGAC
CCAGCTGTGGCTTGTCTCTGACTATCACGAGCATGGCTCACTGTTTGATT
ATCTGAACCGCTACACAGTGACCATTGAGGGAATGATTAAGCTAGCCTTG
TCTGCAGCCAGTGGTTTGGCACACCTGCATATGGAGATTGTGGGCACTCA
AGGGAAGCCGGGAATTGCTCATCGAGACTTGAAGTCAAAGAACATCCTGG
TGAAAAAAAATGGCATGTGTGCCATTGCAGACCTGGGCCTGGCTGTCCGT
CATGATGCGGTCACTGACACCATAGACATTGCTCCAAATCAGAGGGTGGG
GACCAAACGATACATGGCTCCTGAAGTCCTTGACGAGACAATCAACATGA
AGCACTTTGACTCCTTCAAATGTGCCGACATCTATGCCCTCGGGCTTGTC
TACTGGGAGATTGCACGAAGATGCAATTCTGGAGGAGTCCATGAAGACTA
TCAACTGCCGTATTACGACTTAGTGCCCTCCGACCCTTCCATTGAGGAGA
TGCGAAAGGTTGTATGTGACCAGAAGCTACGGCCCAATGTCCCCAACTGG
TGGCAGAGTTATGAGGCCTTGCGAGTGATGGGAAAGATGATGCGGGAGTG
CTGGTACGCCAATGGTGCTGCCCGTCTGACAGCTCTGCGCATCAAGAAGA
CTCTGTCCCAGCTAAGCGTGCAGGAAGATGTGAAGATTTAAGCTGTTCCT
CTGCCTACACAAAGAACCTGGGCAGTGAGGATGACTGCAGCCACCGTGCA
AGCGTCGTGGAGGCCTACCTCTTGTTTCTGCCCGGCCCTCTGGCCAGAGC
CCTGGCCTGCAAGAGGGACAGAGCCTGGGAGACGCGCACTCCCGTTGGGT
TTGAGACAGACACTTTTTATATTTACCTCCTGATGGCATGGAGACTCTGA
GCAAATCATGTAGACAACTCAATGCCACAACTCAAACTGCTTGCAGTGGG
AAGTACAGAGAGCCCAGTGCATCTGGCGTGTTGCCAGGAGCGGTGAAGGG
TGCTGGGCTCGCCCAGGAGCGGCCCCCATACCTTGTGGTCCACTGGGCTG
CAGGTTTTCCTCCAGGGACCAGTCAACTGGCATCAAGATATTGAGAGGAA
CCGGAAGTTTCCTCCCTCCTTCCCGTAGGCAGTCCTGAGCCACACCATCC
CTTCTCATGGACATCCGGAGGGACTGCCCCTAGAGACACAACCTGCTGCC
TGTCTGTCCAGCCAAGTGCGCATGTGCCGAGGTGTGTCCCACATTGTGCC
TGGTCTGTGCCACGCCCGTGTGTGTGTGTGTGTGTGTGAGTGAGTGTGTG
TGTGTACACTTAACCTGCTTGAGCTTTCTGTGCATGTGTAGGCCAGGGTG
TGGTGGCCATACTGTCTCTGAGTGCTGCTGCTTCTCAGTGAGCAGCATGT
AGTTAACCTGGTGCCCTCCTAGGTGTCTCCTGTCCCCAGACCCCATCAGT
CAGGGAGGTTCTGTCTTCTCAGCAGGCTGCTTGCCCACCCTGTGTCACAG
GCCCTCCTCTTCCATTTCAGACCAGAACCAAAGCTGGCCCACTTGTCCAT
GGTAGGAGAAGCTTTTGGGTCAAAATGAGGGGGACTTGATGAGCAGAGAG
AGAATGTAGGTGGAAGTCTTGGGTGCTGTGTTTCAGCATCAGCCATGGGA
AATGAGCCAGCCCAAGGGCATCTTCCTTGACAGCTGTGAGGAAGGGCCGA
GGAATCCGAAGCCAGAGCTTGGGACTCAGATTGGAATGTAACATCTGTTT
ATGTCCCACCCCAGATTCTGCAAACTGCAGTGTATATTTTTGGTGGTGGG
TTTGGGGTGGGAAGGGATGGGTTGCAGGGCGTGGGGAGGGAGGCTGGGGT
GGGAGGGAGGCATCTGCATGGGCTTCTTGTACTGGATTCTCTGATCAGGG
TAGAGAAGAGGCAAGGCTTGCATCCACTTCAGGGTCCCTACTGAGGAGAG
TGAGCGGTCCGAGCTGAATATGGTGTTTAACCTAAGTTTAGTATTTAATG
AATTCTAGAAGCCTGGCTGTGGGTGGTGATTTGGTCAGCATATCTTAGGT
ATATAATAACTTTGAAGCCATAACTTTTTAACTGGAGTGGTTTATTTTAA
TTCAGTTTATTTTATTTTATTTTGGGGGGAGGGTCAGGATTTTAACTTTA
ATATTGTTAAGTTTTGTAAAAGGAGAACCATCTCTGTGACAATTACCTCT
TAGTCTGTTTGTTTTTAAAGAAATCCCTAAAACAAACAAAACACAAAAAT
TCTCCAGACTCAAACGCACATAGTTCAGTCACTGGAAACGCTTGTCTTTG
CACCTGAGCCTGATCCCGCTGAGCAGTGAGGGCTGCTTTTCCCCATGGGG
GCTTGCTGTCTCGTACTCCCTGCACCCTCGGCCCCATCCCGTGAGCACCT
CGGCCCTCTGCACATTGCCCGGCTGGTTGGACCCTTTCCAGATACTTGCT
CAGCAAATGTGGGCTGCGAGCCTGCTGAGCGCTGGCCCGGGAGGATCTCC
TCAGGGTGGGGCAGGCTTGGGCGCTGCTCTGCTCCTCTACCACTGGAGGG
AATGGAATCATGCGATGGGCGAGCACCTGCTGTGGAGACCAGAAGTGCTC
ATGGCTGGTCCTGAGAGCCTTGATGAGCTAGGATCACTGTTCTTAAAGAC
CACTGAAACTGGAAGGGGGACCTGTATCCCCTTGGGAAGAGAAGCCCCTG
GCAAGCAGTGGGTCCTGGAGACTGGGTTCATTGTGAGCCTTTCCTGCCAG
GGGAGGCATGAGTCTTTGCAGGGAAACTGTCTCCTCCAGCTTCTCCTGCC
TTGGTCTCCCCATATTCTTAGCCTTTCTATTTATTTCCTGGTGTATAACT
TTCCTTGCTTTAAAGGGATCTTCCTTTAATTCCTTGGGCCAATTTACTGG
CCATTGAACAGTGTCCCTTGAGTCCCAACTGTGTCTCTGGGGAACCTCCT
TACCCACCCCTGCTGACCTCCCACTTCCCACCCTGCAGCTGAGTATCCGT
GATTACAGGCGATTGAACTGTAGAGTCCTCTCTGCCTCTGTACCTGCCAG
CAGCAGCCTCACAGTGACCCCCACGCCACTGGACAACTCCCAGGAGACCT
GTGCGCTCCGTGCAGCTCAGCTCAGCCGCCTCTCAGGAAGCCTGGAGCAG
GTCTGGGGGACCCCCCCCCCATCACTCTTTACATTAAGCTGAGAGTTGGG
AGAAGCTGTGCTTTGGCTCCCTGAGGCCACCCTGATCCACGGGGCACCCG
CACCTCTGCGTTCTGGATTCACAGACCAAGTCTAAAGCCCGTTCGTTCCT
GAGTTGCCGTCAAGGCCCCTGAACGGTACTCTCGGTACTTCTGTTTGTTT
TTTGTACAATTTATTAGAAAGGACTGTAAAATAGCCACTTAGACACTTTA
CCTCTCCAGTATGCAAATGTAAATATATTGTAATATAGGAAATTTTTGTT
TTAATATAAGAATGAGCCTGTCCAGTTTCTGCTGTACATTATTAAAGTTT
TATTCACAGAACTAAAAAAAAAAAAAAAAAAAAAAAA.

In some embodiments, oligonucleotides may have a region of complementarity to a rat ACVR1 sequence, for example, as provided below (Gene ID: 29381; NCBI Ref. No: NM_199230.1)

(SEQ ID NO: 370)
GGCGGCGGTTACTATGGCGGAGTCGGCCGGAGCCTCCTCCTTCTTCCCCC
TTGTTGTCCTCCTGCTCGCCGGCAGTGGCGGGTCCGGGCCCCGGGGGATC
CAGGCTCTGCTGTGTGCATGCACCAGCTGCCTACAGACCAACTACACCTG
CGAAACAGATGGGGCCTGCATGGTCTCCATCTTTAACCTGGATGGCATGG
AGCACCACGTACGCACCTGCATCCCCAAGGTGGAGCTTGTGCCTGCTGGG
AAGCCCTTCTACTGCCTGAGTTCAGAGGACCTGCGCAACACGCACTGCTG
CTATATTGACTTCTGCAACAAGATTGACCTGAGGGTGCCCAGTGGACACC
TCAAGGAGCCTGAGCACCCCTCCATGTGGGGCCCTGTGGAGCTGGTCGGC
ATCATTGCCGGTCCTGTCTTCCTCCTCTTCCTCATCATCATCATCGTCTT
CCTGGTCATCAACTATCATCAGCGTGTCTACCACAACCGCCAAAGACTGG
ACATGGAGGACCCCTCATGTGAGATGTGTCTCTCCAAAGACAAGACGCTC
CAGGATCTCGTCTACGATCTCTCCACTTCAGGATCGGGCTCAGGGTTACC
CCTTTTTGTCCAGCGCACAGTGGCCCGAACCATTGTTTTACAAGAGATTA
TCGGCAAGGGCCGGTTTGGGGAAGTATGGCGTGGCCGCTGGAGGGGTGGT
GATGTGGCTGTGAAAATCTTCTCTTCCCGTGAAGAGCGGTCGTGGTTCCG
GGAGGCAGAGATCTACCAGACTGTCATGCTGCGCCATGAAAACATCCTTG
GGTTTATTGCTGCTGACAATAAAGACAATGGCACCTGGACCCAGCTGTGG
CTTGTCTCTGACTATCACGAGCACGGCTCACTGTTCGATTATCTGAACCG
CTACACAGTGACCATTGAGGGGATGATTAAACTGGCCCTGTCTGCAGCCA
GTGGTTTGGCACACCTGCATATGGAGATTGTGGGCACTCAGGGGAAGCCT
GGAATTGCTCATCGAGACTTGAAGTCAAAGAACATTCTGGTGAAGAAGAA
TGGCATGTGTGCCATTGCAGACCTGGGCCTAGCTGTCCGTCACGATGCTG
TCACTGACACCATAGACATTGCTCCAAATCAGAGGGTGGGAACCAAACGA
TACATGGCTCCTGAAGTACTTGACGAGACCATCAACATGAAGCACTTTGA
CTCCTTCAAGTGTGCCGATATCTACGCCCTCGGGCTTGTCTATTGGGAGA
TTGCTCGGAGGTGCAATTCTGGAGGAGTCCATGAAGAGTATCAACTGCCA
TATTATGATTTAGTGCCCTCTGACCCTTCCATTGAGGAAATGCGAAAGGT
CGTCTGTGACCAGAAGCTACGGCCCAATGTCCCCAACTGGTGGCAGAGTT
ATGAGGCCTTGCGAGTGATGGGGAAGATGATGCGGGAGTGCTGGTACGCC
AATGGTGCTGCCCGCCTGACAGCGCTGCGCATCAAGAAGACTTTGTCCCA
GCTAAGCGTGCAGGAAGACGTGAAGATTTAAGCTGTTCCTCTGCCTACGC
AAAGAACCTGGGCAGTGAGGATGCCTGCAGCCACCGTGCAAGCGTGGAGG
CCTACCTCTTGTTTCTGCCCAGCCCTCTGGCCAGAGCCCTGGTCTGCAAG
AGGGACAGAGCCTGGGAGACGCACACTCCCTACTGGGTTTGAGACAGACA
CTTTTTATATTTACCTCCTGATGGCATGGAGACTCTGAGAGCAAATCATG
TAGATGACTCGATGCCACAACTCGCACTGCGTGCAGTGGGAAGGACAGAA
AGCCCAGTGCATCTGGCATGTTGCCAGGAGTGGTGATGGGTGCTGGGCTC
GCCTGGGAGCAGCCCCCATACCGTGTTGTCCACTGGACTGCAGGTTTCCT
CCAGGGACCAGTCAACTGGCAGATACTGAGAGGAACCGGAAGTGTCCTCC
CTTTTACCTGTGGGCAGTCCTGAGCCACGCCATCCCCTTCTCATCTGGAG
GACCGCCCCTAGAGACACAACCTGCTGCCTGTCTGTCCAGCCAAGTGCGC
ATGTGCCGAGGTGTGTCTCACATTGTGCCTGGTCCGTGCCTCGCCCGTGT
GTGTGTGTGTGTGTGTGTGTATGTGTGTGTGTAGGTGTGTGTGAGTGTGT
GTGTTAGTGTAGGTGTGTGAGAGTGTGTGTGTAGGTGTGTGAGTGTGGGT
GTGTGAGAGTGTGTGTAGGTGTATGTGAGTGTGTAAGTGTGTGTAGGTGT
GTGAGTGTGTAGGTGTGTGAGTGTG

In some embodiments, the oligonucleotide may have a region of complementarity to a mutant form of ACVR1B, for example as reported in Su, G. H. et al. Proc Nal Acad Sci USA. 2001 Mar. 13; 98(6): 3254-3257., the contents of which are incorporated herein by reference in their entirety.

In some embodiments, an oligonucleotide comprises a region of complementarity to an ACVR1B sequence as set forth in SEQ ID NO: 367, SEQ ID NO: 368, SEQ ID NO: 369, or SEQ ID NO: 370. In some embodiments, the oligonucleotide comprises a region of complementarity that is at least 80%, 85%, 90%, 95%, 96%, 97%, 98%, 99%, or 100% complementary to an ACVR1B sequence as set forth in SEQ ID NO: 367, SEQ ID NO: 368, SEQ ID NO: 369, or SEQ ID NO: 370. In some embodiments, the oligonucleotide comprises a sequence that has at least 10, 11, 12, 13, 14, 15, 16, 17, 18, or 19 consecutive nucleotides that are perfectly complementary to an ACVR1B sequence as set forth in SEQ ID NO: 367, SEQ ID NO: 368, SEQ ID NO: 369, or SEQ ID NO: 370. In some embodiments, an oligonucleotide may comprise a sequence that targets (e.g., is complementary to) an RNA version (i.e., wherein the T's are replaced with U's) of an ACVR1B sequence as set forth in SEQ ID NO: 367, SEQ ID NO: 368, SEQ ID NO: 369, or SEQ ID NO: 370. In some embodiments, the oligonucleotide comprises a sequence that is complementary (e.g., at least 80%, 85%, 90%, 95%, 96%, 97%, 98%, 99%, or 100% complementary) to an RNA version of an ACVR1B sequence as set forth in SEQ ID NO: 367, SEQ ID NO: 368, SEQ ID NO: 369, or SEQ ID NO: 370. In some embodiments, the oligonucleotide comprises a sequence that has at least 10, 11, 12, 13, 14, 15, 16, 17, 18, or 19 consecutive nucleotides that are perfectly complementary to an RNA version of an ACVR1B sequence as set forth in SEQ ID NO: 367, SEQ ID NO: 368, SEQ ID NO: 369, or SEQ ID NO: 370.

In some embodiments, an ACVR1B-targeting oligonucleotide comprises an antisense strand that comprises at least 10, 11, 12, 13, 14, 15, 16, 17, 18, or 19 consecutive nucleotides of a sequence comprising any one of SEQ ID NOs: 343-366. In some embodiments, an ACVR1B-targeting oligonucleotide comprises an antisense strand that comprises any one of SEQ ID NO: 343-366. In some embodiments, an oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 343-366.

In some embodiments, an ACVR1B-targeting oligonucleotide comprises an antisense strand that targets an ACVR1B sequence comprising any one of SEQ ID NO:. In some embodiments, an oligonucleotide comprises an antisense strand comprising at least 10, 11, 12, 13, 14, 15, 16, 17, 18, or 19 nucleotides (e.g., consecutive nucleotides) that are complementary to an ACVR1B sequence comprising any one of SEQ ID NO: 221-268. In some embodiments, an ACVR1B-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NO: 221-268.

In some embodiments, an ACVR1B-targeting oligonucleotide comprises an antisense strand that comprises a region of complementarity to a target sequence as set forth in any one of SEQ ID NOs: 221-268. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, an ACVR1B-targeting oligonucleotide further comprises a sense strand that hybridizes to the antisense strand to form a double stranded siRNA. In some embodiments, the ACVR1B-targeting oligonucleotide comprises an antisense strand that comprises the nucleotide sequence of any one of SEQ ID NOs: 343-366. In some embodiments, the ACVR1B-targeting oligonucleotide further comprises a sense strand that comprises the nucleotide sequence of any one of SEQ ID NOs: 245-268.

In some embodiments, the ACVR1B-targeting oligonucleotide is a double stranded oligonucleotide (e.g., an siRNA) comprising an antisense strand that comprises the nucleotide sequence of any one of SEQ ID NOs: 343-366 and a sense strand that hybridizes to the antisense strand and comprises the nucleotide sequence of any one of SEQ ID NOs: 245-268, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the ACVR1B-targeting oligonucleotide is a double stranded oligonucleotide (e.g., an siRNA) comprising an antisense strand that comprises the nucleotide sequence of any one of SEQ ID NOs: 343-366 and a sense strand that hybridizes to the antisense strand and comprises the nucleotide sequence of any one of SEQ ID NOs: 245-268, wherein each nucleoside in the antisense strand and/or (e.g., and) each nucleoside in the sense strand is a 2′-modified nucleoside selected from 2′-O-Me and 2′-F modified nucleosides.

In some embodiments, the ACVR1B-targeting oligonucleotide is a double stranded oligonucleotide (e.g., an siRNA) comprising an antisense strand that comprises the nucleotide sequence of any one of SEQ ID NOs: 343-366 and a sense strand that hybridizes to the antisense strand and comprises the nucleotide sequence of any one of SEQ ID NOs: 245-268, wherein each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the ACVR1B-targeting oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates a phosphorothioate internucleoside linkage; and the absence of “*” between two nucleosides indicates a phosphodiester internucleoside linkage.

In some embodiments, the sense strand of the ACVR1B-targeting oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicates a phosphodiester internucleoside linkage.

In some embodiments, the antisense strand of the ACVR1B-targeting oligonucleotide is selected from the modified version of SEQ ID NOs: 343-366 listed in Table 16. In some embodiments, the sense strand of the ACVR1B-targeting oligonucleotide is selected from the modified version of SEQ ID NOs: 245-268 listed in Table 16. In some embodiments, the ACVR1B-targeting oligonucleotide is an siRNA selected from the siRNAs listed in Table 16.

TABLE 14
ACVRIB Target Sequences

| | Corresponding | | |
|---|---|---|---|
| | nucleotides | ACVR1B Target | SEQ |
| Reference | of Reference | Sequence | ID |
| sequence | Sequence | (5′ to 3′) | NO: |
| NM_004302.5 | 572-591 | ACAAGACGCTCCA | 221 |
| (SEQ ID NO: 367) | | GGATCT | |
| NM_004302.5 | 1034-1053 | GAATTGCTCATCG | 222 |
| (SEQ ID NO: 367) | | AGACTT | |
| NM_004302.5 | 1418-1437 | ACTGGTGGCAGAG | 223 |
| (SEQ ID NO: 367) | | TTATGA | |
| NM_004302.5 | 1294-1313 | TGCAATTCTGGAG | 224 |
| (SEQ ID NO: 367) | | GAGTCC | |
| NM_004302.5 | 565-584 | TCCAAAGACAAGA | 225 |
| (SEQ ID NO: 367) | | CGCTCC | |
| NM_004302.5 | 2970-2989 | ATAATAACTTTGA | 226 |
| (SEQ ID NO: 367) | | AGCCAT | |
| NM_004302.5 | 2984-3003 | GCCATAACTTTTA | 227 |
| (SEQ ID NO: 367) | | ACTGGA | |
| NM_004302.5 | 4463-4482 | CTTTTGTTTTAAT | 228 |
| (SEQ ID NO: 367) | | ATAAGA | |
| NM_004302.5 | 582-601 | CCAGGATCTTGTC | 229 |
| (SEQ ID NO: 367) | | TACGAT | |
| NM_004302.5 | 905-924 | ACGGGTCCCTGTT | 230 |
| (SEQ ID NO: 367) | | TGATTA | |
| NM_004302.5 | 212-231 | TTTTCAATCTGGA | 231 |
| (SEQ ID NO: 367) | | TGGGAT | |
| NM_004302.5 | 4435-4454 | AATGTAAATAAAT | 232 |
| (SEQ ID NO: 367) | | TGTAAT | |
| NM_004302.5 | 473-491 | TCATTGTTTTCCT | 233 |
| (SEQ ID NO: 367) | | TGTCAT | |
| NM_004302.5 | 1535-1554 | TCAGCGTGCAGGA | 234 |
| (SEQ ID NO: 367) | | AGACGT | |
| NM_004302.5 | 1784-1803 | AGAGCGAATTGTG | 235 |
| (SEQ ID NO: 367) | | TGGAGA | |
| NM_199230.1 | 1401-1420 | ATGAGGCCTTGCG | 236 |
| (SEQ ID NO: 370) | | AGTGAT | |
| NM_199230.1 | 964-983 | CCTGCATATGGAG | 237 |
| (SEQ ID NO: 370) | | ATTGTG | |
| NM_199230.1 | 2046-2065 | TGCGCATGTGCCG | 238 |
| (SEQ ID NO: 370) | | AGGTGT | |
| NM_199230.1 | 1104-1123 | CTGACACCATAGA | 239 |
| (SEQ ID NO: 370) | | CATTGC | |
| NM_199230.1 | 293-312 | CACTGCTGCTATA | 240 |
| (SEQ ID NO: 370) | | TTGACT | |
| NM_199230.1 | 174-193 | TCTCCATCTTTAA | 241 |
| (SEQ ID NO: 370) | | CCTGGA | |
| NM_199230.1 | 1654-1673 | GACAGAGCCTGGG | 242 |
| (SEQ ID NO: 370) | | AGACGC | |
| NM_199230.1 | 970-989 | TATGGAGATTGTG | 243 |
| (SEQ ID NO: 370) | | GGCACT | |
| NM_199230.1 | 642-661 | AAGAGATTATCGG | 244 |
| (SEQ ID NO: 370) | | CAAGGG | |

* The target sequences contain Ts, but binding to RNA and/or DNA is contemplated.

In some embodiments, an oligonucleotide may comprise or consist of any sequence as provided in Table 15.

TABLE 15
Oligonucleotide sequences for targeting ACVRIB

| Passenger Strand/ | SEQ | Guide Strand/ | SEQ |
|---|---|---|---|
| Sense Strand | ID | Antisense Strand | ID |
| (RNA) (5′ to 3′) | NO: | (RNA) (5′ to 3′) | NO: |
| AGACAAGACGCUCCAGGAUCU | 245 | AGAUCCUGGAGCGUCUUGUCUUU | 343 |
| UGGAAUUGCUCAUCGAGACUU | 246 | AAGUCUCGAUGAGCAAUUCCAGG | 344 |
| CAACUGGUGGCAGAGUUAUGA | 247 | UCAUAACUCUGCCACCAGUUGGG | 345 |
| GAUGCAAUUCUGGAGGAGUCC | 248 | GGACUCCUCCAGAAUUGCAUCUU | 346 |
| UCUCCAAAGACAAGACGCUCC | 249 | GGAGCGUCUUGUCUUUGGAGAGA | 347 |
| AUAUAAUAACUUUGAAGCCAU | 250 | AUGGCUUCAAAGUUAUUAUAUAC | 348 |
| AAGCCAUAACUUUUAACUGGA | 251 | UCCAGUUAAAAGUUAUGGCUUCA | 349 |
| AUCUUUUGUUUUAAUAUAAGA | 252 | UCUUAUAUUAAAACAAAAGAUUU | 350 |
| CUCCAGGAUCUUGUCUACGAU | 253 | AUCGUAGACAAGAUCCUGGAGCG | 351 |
| GCACGGGUCCCUGUUUGAUUA | 254 | UAAUCAAACAGGGACCCGUGCUC | 352 |
| CAUUUUCAAUCUGGAUGGGAU | 255 | AUCCCAUCCAGAUUGAAAAUGGA | 353 |
| CAAAUGUAAAUAAAUUGUAAU | 256 | AUUACAAUUUAUUUACAUUUGCA | 354 |
| CAUCAUUGUUUUCCUUGUCAU | 257 | AUGACAAGGAAAACAAUGAUGAU | 355 |
| GCUCAGCGUGCAGGAAGACGU | 258 | ACGUCUUCCUGCACGCUGAGCUG | 356 |
| UGAGAGCGAAUUGUGUGGAGA | 259 | UCUCCACACAAUUCGCUCUCAGA | 357 |
| UUAUGAGGCCUUGCGAGUGAU | 260 | AUCACUCGCAAGGCCUCAUAACU | 358 |
| CACCUGCAUAUGGAGAUUGUG | 261 | CACAAUCUCCAUAUGCAGGUGUG | 359 |
| AGUGCGCAUGUGCCGAGGUGU | 262 | ACACCUCGGCACAUGCGCACUUG | 360 |
| CACUGACACCAUAGACAUUGC | 263 | GCAAUGUCUAUGGUGUCAGUGAC | 361 |
| CACACUGCUGCUAUAUUGACU | 264 | AGUCAAUAUAGCAGCAGUGUGUG | 362 |
| GGUCUCCAUCUUUAACCUGGA | 265 | UCCAGGUUAAAGAUGGAGACCAU | 363 |
| GGGACAGAGCCUGGGAGACGC | 266 | GCGUCUCCCAGGCUCUGUCCCUC | 364 |
| CAUAUGGAGAUUGUGGGCACU | 267 | AGUGCCCACAAUCUCCAUAUGCA | 365 |
| ACAAGAGAUUAUCGGCAAGGG | 268 | CCCUUGCCGAUAAUCUCUUGUAA | 366 |

In some embodiments, an oligonucleotide is a modified oligonucleotide as provided in Table 16, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 16
Modified Oligonucleotides for targeting ACVRIB

| | Modified | | Modified | |
|---|---|---|---|---|
| | Passenger Strand/ | | Guide Strand/ | |
| | Sense Strand | SEQ ID | Antisense Strand | SEQ ID |
| siRNA # | (RNA) (5′ to 3′) | NO: | (RNA) (5′ to 3′) | NO: |
| hsACVR1B-3 | mAmGfAmCfAmAfGmAfCm | 245 | fAfGmAfUmCfCmUfGmGf | 343 |
| | GfCmUfCmCfAmGfGmAfU | | AmGfCmGfUmCfUmUfGm | |
| | mCfU | | UfCmU*fU*mU | |
| hsACVR1B-4 | mUmGfGmAfAmUfUmGfCm | 246 | fAfAmGfUmCfUmCfGmAf | 344 |
| | UfCmAfUmCfGmAfGmAfC | | UmGfAmGfCmAfAmUfUm | |
| | mUfU | | CfCmA*fG*mG | |
| hsACVR1B-5 | mCmAfAmCfUmGfGmUfGm | 247 | fUfCmAfUmAfAmCfUmCf | 345 |
| | GfCmAfGmAfGmUfUmAfU | | UmGfCmCfAmCfCmAfGm | |
| | mGfA | | UfUmG*fG*mG | |
| hsACVR1B-6 | mGmAfUmGfCmAfAmUfUm | 248 | fGfGmAfCmUfCmCfUmCf | 346 |
| | CfUmGfGmAfGmGfAmGfU | | CmAfGmAfAmUfUmGfCm | |
| | mCfC | | AfUmC*fU*mU | |
| hsACVR1B-7 | mUmCfUmCfCmAfAmAfGm | 249 | fGfGmAfGmCfGmUfCmUf | 347 |
| | AfCmAfAmGfAmCfGmCfU | | UmGfUmCfUmUfUmGfGm | |
| | mCfC | | AfGmA*fG*mA | |
| hsACVR1B-8 | mAmUfAmUfAmAfUmAfAm | 250 | fAfUmGfGmCfUmUfCmAf | 348 |
| | CfUmUfUmGfAmAfGmCfC | | AmAfGmUfUmAfUmUfA | |
| | mAfU | | mUfAmU*fA*mC | |
| hsACVR1B-9 | mAmAfGmCfCmAfUmAfAm | 251 | fUfCmCfAmGfUmUfAmAf | 349 |
| | CfUmUfUmUfAmAfCmUfG | | AmAfGmUfUmAfUmGfG | |
| | mGfA | | mCfUmU*fC*mA | |
| hsACVR1B-10 | mAmUfCmUfUmUfUmGfUm | 252 | fUfCmUfUmAfUmAfUmUf | 350 |
| | UfUmUfAmAfUmAfUmAfA | | AmAfAmAfCmAfAmAfAm | |
| | mGfA | | GfAmU*fU*mU | |
| hsACVR1B-1 | mCmUfCmCfAmGfGmAfUm | 253 | fAfUmCfGmUfAmGfAmCf | 351 |
| | CfUmUfGmUfCmUfAmCfG | | AmAfGmAfUmCfCmUfGm | |
| | mAfU | | GfAmG*fC*mG | |
| hsACVR1B-11 | mGmCfAmCfGmGfGmUfCm | 254 | fUfAmAfUmCfAmAfAmCf | 352 |
| | CfCmUfGmUfUmUfGmAfU | | AmGfGmGfAmCfCmCfGm | |
| | mUfA | | UfGmC*fU*mC | |
| hsACVR1B-12 | mCmAfUmUfUmUfCmAfAm | 255 | fAfUmCfCmCfAmUfCmCf | 353 |
| | UfCmUfGmGfAmUfGmGfG | | AmGfAmUfUmGfAmAfA | |
| | mAfU | | mAfUmG*fG*mA | |
| hsACVR1B-13 | mCmAfAmAfUmGfUmAfAm | 256 | fAfUmUfAmCfAmAfUmUf | 354 |
| | AfUmAfAmAfUmUfGmUfA | | UmAfUmUfUmAfCmAfUm | |
| | mAfU | | UfUmG*fC*mA | |
| hsACVR1B-2 | mCmAfUmCfAmUfUmGfUm | 257 | fAfUmGfAmCfAmAfGmGf | 355 |
| | UfUmUfCmCfUmUfGmUfC | | AmAfAmAfCmAfAmUfGm | |
| | mAfU | | AfUmG*fA*mU | |
| hsACVR1B-14 | mGmCfUmCfAmGfCmGfUm | 258 | fAfCmGfUmCfUmUfCmCf | 356 |
| | GfCmAfGmGfAmAfGmAfC | | UmGfCmAfCmGfCmUfGm | |
| | mGfU | | AfGmC*fU*mG | |
| hsACVR1B-15 | mUmGfAmGfAmGfCmGfAm | 259 | fUfCmUfCmCfAmCfAmCf | 357 |
| | AfUmUfGmUfGmUfGmGfA | | AmAfUmUfCmGfCmUfCm | |
| | mGfA | | UfCmA*fG*mA | |
| mmACVR1B-4 | mUmUfAmUfGmAfGmGfCm | 260 | fAfUmCfAmCfUmCfGmCf | 358 |
| | CfUmUfGmCfGmAfGmUfG | | AmAfGmGfCmCfUmCfAm | |
| | mAfU | | UfAmA*fC*mU | |
| mmACVR1B-5 | mCmAfCmCfUmGfCmAfUm | 261 | fCfAmCfAmAfUmCfUmCf | 359 |
| | AfUmGfGmAfGmAfUmUfG | | CmAfUmAfUmGfCmAfGm | |
| | mUfG | | GfUmG*fU*mG | |
| mmACVR1B-6 | mAmGfUmGfCmGfCmAfUm | 262 | fAfCmAfCmCfUmCfGmGf | 360 |
| | GfUmGfCmCfGmAfGmGfU | | CmAfCmAfUmGfCmGfCm | |
| | mGfU | | AfCmU*fU*mG | |
| mmACVR1B-7 | mCmAfCmUfGmAfCmAfCm | 263 | fGfCmAfAmUfGmUfCmUf | 361 |
| | CfAmUfAmGfAmCfAmUfU | | AmUfGmGfUmGfUmCfAm | |
| | mGfC | | GfUmG*fA*mC | |
| mmACVR1B-1 | mCmAfCmAfCmUfGmCfUm | 264 | fAfGmUfCmAfAmUfAmUf | 362 |
| | GfCmUfAmUfAmUfUmGfA | | AmGfCmAfGmCfAmGfUm | |
| | mCfU | | GfUmG*fU*mG | |
| mmACVR1B-2 | mGmGfUmCfUmCfCmAfUm | 265 | fUfCmCfAmGfGmUfUmAf | 363 |
| | CfUmUfUmAfAmCfCmUfG | | AmAfGmAfUmGfGmAfG | |
| | mGfA | | mAfCmC*fA*mU | |
| mmACVR1B-8 | mGmGfGmAfCmAfGmAfGm | 266 | fGfCmGfUmCfUmCfCmCf | 364 |
| | CfCmUfGmGfGmAfGmAfC | | AmGfGmCfUmCfUmGfUm | |
| | mGfC | | CfCmC*fU*mC | |
| mmACVR1B-9 | mCmAfUmAfUmGfGmAfGm | 267 | fAfGmUfGmCfCmCfAmCf | 365 |
| | AfUmUfGmUfGmGfGmCfA | | AmAfUmCfUmCfCmAfUm | |
| | mCfU | | AfUmG*fC*mA | |
| mmACVR1B-3 | mAmCfAmAfGmAfGmAfUm | 268 | fCfCmCfUmUfGmCfCmGf | 366 |
| | UfAmUfCmGfGmCfAmAfG | | AmUfAmAfUmCfUmCfUm | |
| | mGfG | | UfGmU*fA*mA | |

d. MLCK1 Oligonucleotides

In some embodiments, the oligonucleotide is an antisense oligonucleotide (ASO). In some embodiments, the oligonucleotide is a siRNA. In some embodiments, the oligonucleotide is a short hairpin RNA. In some embodiments, the oligonucleotide is a miRNA-based shRNA (e.g., based on miR-155 or miR-200c). In some embodiments, the oligonucleotide is a CRISPR guide RNA targeting MLCK1. In some embodiments, the oligonucleotide inhibits the expression or function of MLCK1.

Examples of oligonucleotides useful for targeting MLCK1 are provided in Weber M. et al. “MiRNA-155 targets myosin light chain kinase and modulates actin cytoskeleton organization in endothelial cells.” Am J Physiol Heart Circ Physiol. 2014 Apr. 15; 306(8):H1192-203.; Thatcher S. E. et al. “Myosin light chain kinase/actin interaction in phorbol dibutyrate-stimulated smooth muscle cells.” J Pharmacol Sci. 2011; 116(1):116-27.; Kohama K. and Nakamura A. “Targeting of myosin light chain kinase in vascular smooth muscle cells, and its implication for drug discovery.” Nihon Yakurigaku Zasshi. 2001 October; 118(4):269-76.; and U.S. Patent Application Publication 2010/0093830, entitled Modulation of MLCK-L expression and uses thereof,” published on Apr. 15, 2010; the contents of each of which are incorporated herein in their entireties.

In some embodiments, oligonucleotides may have a region of complementarity to a human MLCK1 sequence, for example, as provided below (Gene ID: 4638; NCBI Ref. No: NM_053025.4):

(SEQ ID NO: 411)
GGGCTCCCCGCGCCGCCCGGTCGGCAGCAGGGCGCTGAGCGAGCTCGGAGCCCGCGCTGTGCGC
CTGCGGCCGGGGCGCCCCGCCGAGCGCCGGTGCCCCGGCTCCCGGGCCGCCTTCGCCGCGCGGG
AAGGATTCTTCAAAATTAACAGAAACCAATTCGGGCCAGCTGAAGAGAAAAAATAAAGGTGGCT
CCCGGCTGCCTCTGCTGCAGTTCAGAGCAACTTCAGGAGCTTCCCAGCCGAGAGCTTCAGGACG
CCTTTCCTGTCCCACTGGCCCAGTTGCCACAACAAACAACAGAGAAGACGGTGACCATGGGGGA
TGTGAAGCTGGTTGCCTCGTCACACATTTCCAAAACCTCCCTCAGTGTGGATCCCTCAAGAGTT
GACTCCATGCCCCTGACAGAGGCCCCTGCTTTCATTTTGCCCCCTCGGAACCTCTGCATCAAAG
AAGGAGCCACCGCCAAGTTCGAAGGGCGGGTCCGGGGTTACCCAGAGCCCCAGGTGACATGGCA
CAGAAACGGGCAACCCATCACCAGCGGGGGCCGCTTCCTGCTGGATTGCGGCATCCGGGGGACT
TTCAGCCTTGTGATTCATGCTGTCCATGAGGAGGACAGGGGAAAGTATACCTGTGAAGCCACCA
ATGGCAGTGGTGCTCGCCAGGTGACAGTGGAGTTGACAGTAGAAGGAAGTTTTGCGAAGCAGCT
TGGTCAGCCTGTTGTTTCCAAAACCTTAGGGGATAGATTTTCAGCTCCAGCAGTGGAGACCCGT
CCTAGCATCTGGGGGGAGTGCCCACCAAAGTTTGCTACCAAGCTGGGCCGAGTTGTGGTCAAAG
AAGGACAGATGGGACGATTCTCCTGCAAGATCACTGGCCGGCCCCAACCGCAGGTCACCTGGCT
CAAGGGAAATGTTCCACTGCAGCCGAGTGCCCGTGTGTCTGTGTCTGAGAAGAACGGCATGCAG
GTTCTGGAAATCCATGGAGTCAACCAAGATGACGTGGGAGTGTACACGTGCCTGGTGGTGAACG
GGTCGGGGAAGGCCTCGATGTCAGCTGAACTTTCCATCCAAGGTTTGGACAGTGCCAATAGGTC
ATTTGTGAGAGAAACAAAAGCCACCAATTCAGATGTCAGGAAAGAGGTGACCAATGTAATCTCA
AAGGAGTCGAAGCTGGACAGTCTGGAGGCTGCAGCCAAAAGCAAGAACTGCTCCAGCCCCCAGA
GAGGTGGCTCCCCACCCTGGGCTGCAAACAGCCAGCCTCAGCCCCCAAGGGAGTCCAAGCTGGA
GTCATGCAAGGACTCGCCCAGAACGGCCCCGCAGACCCCGGTCCTTCAGAAGACTTCCAGCTCC
ATCACCCTGCAGGCCGCAAGAGTTCAGCCGGAACCAAGAGCACCAGGCCTGGGGGTCCTATCAC
CTTCTGGAGAAGAGAGGAAGAGGCCAGCTCCTCCCCGTCCAGCCACCTTCCCCACCAGGCAGCC
TGGCCTGGGGAGCCAAGATGTTGTGAGCAAGGCTGCTAACAGGAGAATCCCCATGGAGGGCCAG
AGGGATTCAGCATTCCCCAAATTTGAGAGCAAGCCCCAAAGCCAGGAGGTCAAGGAAAATCAAA
CTGTCAAGTTCAGATGTGAAGTTTCCGGGATTCCAAAGCCTGAAGTGGCCTGGTTCCTGGAAGG
CACCCCCGTGAGGAGACAGGAAGGCAGCATTGAGGTTTATGAAGATGCTGGCTCCCATTACCTC
TGCCTGCTGAAAGCCCGGACCAGGGACAGTGGGACATACAGCTGCACTGCTTCCAACGCCCAAG
GCCAGCTGTCCTGTAGCTGGACCCTCCAAGTGGAAAGGCTTGCCGTGATGGAGGTGGCCCCCTC
CTTCTCCAGTGTCCTGAAGGACTGCGCTGTTATTGAGGGCCAGGATTTTGTGCTGCAGTGCTCC
GTACGGGGGACCCCAGTGCCCCGGATCACTTGGCTGCTGAATGGGCAGCCCATCCAGTACGCTC
GCTCCACCTGCGAGGCCGGCGTGGCTGAGCTCCACATCCAGGATGCCCTGCCGGAGGACCATGG
CACCTACACCTGCCTAGCTGAGAATGCCTTGGGGCAGGTGTCCTGCAGCGCCTGGGTCACCGTC
CATGAAAAGAAGAGTAGCAGGAAGAGTGAGTACCTTCTGCCTGTGGCTCCCAGCAAGCCCACTG
CACCCATCTTCCTGCAGGGCCTCTCTGATCTCAAAGTCATGGATGGAAGCCAGGTCACTATGAC
TGTCCAAGTGTCAGGGAATCCACCCCCTGAAGTCATCTGGCTGCACAATGGGAATGAGATCCAA
GAGTCAGAGGACTTCCACTTTGAACAGAGAGGAACTCAGCACAGCCTTTGTATCCAGGAAGTGT
TCCCGGAGGACACGGGCACGTACACCTGCGAGGCCTGGAACAGCGCTGGAGAGGTCCGCACCCA
GGCCGTGCTCACGGTACAAGAGCCTCACGATGGCACCCAGCCCTGGTTCATCAGTAAGCCTCGC
TCAGTGACAGCCTCCCTGGGCCAGAGTGTCCTCATCTCCTGCGCCATAGCTGGTGACCCCTTTC
CTACCGTGCACTGGCTCAGAGATGGCAAAGCCCTCTGCAAAGACACTGGCCACTTCGAGGTGCT
TCAGAATGAGGACGTGTTCACCCTGGTTCTAAAGAAGGTGCAGCCCTGGCATGCCGGCCAGTAT
GAGATCCTGCTCAAGAACCGGGTTGGCGAATGCAGTTGCCAGGTGTCACTGATGCTACAGAACA
GCTCTGCCAGAGCCCTTCCACGGGGGAGGGAGCCTGCCAGCTGCGAGGACCTCTGTGGTGGAGG
AGTTGGTGCTGATGGTGGTGGTAGTGACCGCTATGGGTCCCTGAGGCCTGGCTGGCCAGCAAGA
GGGCAGGGTTGGCTAGAGGAGGAAGACGGCGAGGACGTGCGAGGGGTGCTGAAGAGGCGCGTGG
AGACGAGGCAGCACACTGAGGAGGCGATCCGCCAGCAGGAGGTGGAGCAGCTGGACTTCCGAGA
CCTCCTGGGGAAGAAGGTGAGTACAAAGACCCTATCGGAAGACGACCTGAAGGAGATCCCAGCC
GAGCAGATGGATTTCCGTGCCAACCTGCAGCGGCAAGTGAAGCCAAAGACTGTGTCTGAGGAAG
AGAGGAAGGTGCACAGCCCCCAGCAGGTCGATTTTCGCTCTGTCCTGGCCAAGAAGGGGACTTC
CAAGACCCCCGTGCCTGAGAAGGTGCCACCGCCAAAACCTGCCACCCCGGATTTTCGCTCAGTG
CTGGGTGGCAAGAAGAAATTACCAGCAGAGAATGGCAGCAGCAGTGCCGAGACCCTGAATGCCA
AGGCAGTGGAGAGTTCCAAGCCCCTGAGCAATGCACAGCCTTCAGGGCCCTTGAAACCCGTGGG
CAACGCCAAGCCTGCTGAGACCCTGAAGCCAATGGGCAACGCCAAGCCTGCCGAGACCCTGAAG
CCCATGGGCAATGCCAAGCCTGATGAGAACCTGAAATCCGCTAGCAAAGAAGAACTCAAGAAAG
ACGTTAAGAATGATGTGAACTGCAAGAGAGGCCATGCAGGGACCACAGATAATGAAAAGAGATC
AGAGAGCCAGGGGACAGCCCCAGCCTTCAAGCAGAAGCTGCAAGATGTTCATGTGGCAGAGGGC
AAGAAGCTGCTGCTCCAGTGCCAGGTGTCTTCTGACCCCCCAGCCACCATCATCTGGACGCTGA
ACGGAAAGACCCTCAAGACCACCAAGTTCATCATCCTCTCCCAGGAAGGCTCACTCTGCTCCGT
CTCCATCGAGAAGGCACTGCCTGAGGACAGAGGCTTATACAAGTGTGTAGCCAAGAATGACGCT
GGCCAGGCGGAGTGCTCCTGCCAAGTCACCGTGGATGATGCTCCAGCCAGTGAGAACACCAAGG
CCCCAGAGATGAAATCCCGGAGGCCCAAGAGCTCTCTTCCTCCCGTGCTAGGAACTGAGAGTGA
TGCGACTGTGAAAAAGAAACCTGCCCCCAAGACACCTCCGAAGGCAGCAATGCCCCCTCAGATC
ATCCAGTTCCCTGAGGACCAGAAGGTACGCGCAGGAGAGTCAGTGGAGCTGTTTGGCAAAGTGA
CAGGCACTCAGCCCATCACCTGTACCTGGATGAAGTTCCGAAAGCAGATCCAGGAAAGCGAGCA
CATGAAGGTGGAGAACAGCGAGAATGGCAGCAAGCTCACCATCCTGGCCGCGCGCCAGGAGCAC
TGCGGCTGCTACACACTGCTGGTGGAGAACAAGCTGGGCAGCAGGCAGGCCCAGGTCAACCTCA
CTGTCGTGGATAAGCCAGACCCCCCAGCTGGCACACCTTGTGCCTCTGACATTCGGAGCTCCTC
ACTGACCCTGTCCTGGTATGGCTCCTCATATGATGGGGGCAGTGCTGTACAGTCCTACAGCATC
GAGATCTGGGACTCAGCCAACAAGACGTGGAAGGAACTAGCCACATGCCGCAGCACCTCTTTCA
ACGTCCAGGACCTGCTGCCTGACCACGAATATAAGTTCCGTGTACGTGCAATCAACGTGTATGG
AACCAGTGAGCCAAGCCAGGAGTCTGAACTCACAACGGTAGGAGAGAAACCTGAAGAGCCGAAG
GATGAAGTGGAGGTGTCAGATGATGATGAGAAGGAGCCCGAGGTTGATTACCGGACAGTGACAA
TCAATACTGAACAAAAAGTATCTGACTTCTACGACATTGAGGAGAGATTAGGATCTGGGAAATT
TGGACAGGTCTTTCGACTTGTAGAAAAGAAAACTCGAAAAGTCTGGGCAGGGAAGTTCTTCAAG
GCATATTCAGCAAAAGAGAAAGAGAATATCCGGCAGGAGATTAGCATCATGAACTGCCTCCACC
ACCCTAAGCTGGTCCAGTGTGTGGATGCCTTTGAAGAAAAGGCCAACATCGTCATGGTCCTGGA
GATCGTGTCAGGAGGGGAGCTGTTTGAGCGCATCATTGACGAGGACTTTGAGCTGACGGAGCGT
GAGTGCATCAAGTACATGCGGCAGATCTCGGAGGGAGTGGAGTACATCCACAAGCAGGGCATCG
TGCACCTGGACCTCAAGCCGGAGAACATCATGTGTGTCAACAAGACGGGCACCAGGATCAAGCT
CATCGACTTTGGTCTGGCCAGGAGGCTGGAGAATGCGGGGTCTCTGAAGGTCCTCTTTGGCACC
CCAGAATTTGTGGCTCCTGAAGTGATCAACTATGAGCCCATCGGCTACGCCACAGACATGTGGA
GCATCGGGGTCATCTGCTACATCCTAGTCAGTGGCCTTTCCCCCTTCATGGGAGACAACGATAA
CGAAACCTTGGCCAACGTTACCTCAGCCACCTGGGACTTCGACGACGAGGCATTCGATGAGATC
TCCGACGATGCCAAGGATTTCATCAGCAATCTGCTGAAGAAAGATATGAAAAACCGCCTGGACT
GCACGCAGTGCCTTCAGCATCCATGGCTAATGAAAGATACCAAGAACATGGAGGCCAAGAAACT
CTCCAAGGACCGGATGAAGAAGTACATGGCAAGAAGGAAATGGCAGAAAACGGGCAATGCTGTG
AGAGCCATTGGAAGACTGTCCTCTATGGCAATGATCTCAGGGCTCAGTGGCAGGAAATCCTCAA
CAGGGTCACCAACCAGCCCGCTCAATGCAGAAAAACTAGAATCTGAAGAAGATGTGTCCCAAGC
TTTCCTTGAGGCTGTTGCTGAGGAAAAGCCTCATGTAAAACCCTATTTCTCTAAGACCATTCGC
GATTTAGAAGTIGTGGAGGGAAGTGCTGCTAGATTTGACTGCAAGATTGAAGGATACCCAGACC
CCGAGGTTGTCTGGTTCAAAGATGACCAGTCAATCAGGGAGTCCCGCCACTTCCAGATAGACTA
CGATGAGGACGGGAACTGCTCTTTAATTATTAGTGATGTTTGCGGGGATGACGATGCCAAGTAC
ACCTGCAAGGCTGTCAACAGTCTTGGAGAAGCCACCTGCACAGCAGAGCTCATTGTGGAAACGA
TGGAGGAAGGTGAAGGGGAAGGGGAAGAGGAAGAAGAGTGAAACAAAGCCAGAGAAAAGCAGTT
TCTAAGTCATATTAAAAGGACTATTTCTCTAAAACTCAAAAAAAAAAAAAAAACTCAAGATAGT
AAAAGCACCTAGTGTGATAGATTATCGGTTAGGTCATTTGTGGGTTGATTCTTCAGAAACAGCA
GTTGATACCTAGCAGCGTTATTGATGGGCATTAATCTATGTTAGTTGGCACCTTAAGATACTAG
TGCAGCTAGATTTCATTTAGGGAAATCACCAGTAACTTGACTGACCAATTGATTTTAGAGAGAA
AGTAACCAAACCAAATATTTATCTGGGCAAAGTCATAAATTCTCCACTTGAATGCGCTCATGAA
AAATAAGGCCAAAACAAGAGTTCTGGGCCACAGCTCAGCCCAGAGGGTTCCTGGGGATGGGAGG
CCTCTCTCTCCCCACCCCCTGACTCTAGAGAACTGGGTTTTCTCCCAGTACTCCAGCAATTCAT
TTCTGAAAGCAGTTGAGCCACTTTATTCCAAAGTACACTGCAGATGTTCAAACTCTCCATTTCT
CTTTCCCCTTCCACCTGCCAGTTTTGCTGACTCTCAACTTGTCATGAGTGTAAGCATTAAGGAC
ATTATGCTTCTTCGATTCTGAAGACAGGTCCCTGCTCATGGATGACTCTGGCTTCCTTAGGAAA
ATATTTTTCTTCCAAAATCAGTAGGAAATCTAAACTTATCCCCTCTTTGCAGATGTCTAGCAGC
TTCAGACATTTGGTTAAGAACCCATGGGAAAAAAAAAATCCTTGCTAATGTGGTTTCCTTTGTA
AACCAGGATTCTTATTTGTGCTGTTATAGAATATCAGCTCTGAACGTGTGGTAAAGATTTTTGT
GTTTGAATATAGGAGAAATCAGTTTGCTGAAAAGTTAGTCTTAATTATCTATTGGCCACGATGA
AACAGATTTCAACTGATAAAGAGCTGGAGAACTCCATGTACTTTGGAATCTCCTCCAAGATAGC
CAGAGTTTAATACATCTTCATTCTCAACACTCTCCAAAGAACTTGACCTACCTTATGGGTTCCA
TATTTTTCTTCTTAAATGTGCATCAATCATGCCTTGCCCCCAACCTTTAAATATATTCTTAGAC
CTGGTAAATGCACTCAGACTTGCGTCTTTAGGAATTTTTAACTTTCTTTCACTACATTGGCACT
TAAATTTTTTCTTTATAAAGCTTTTTGAAGGTCATAAACAAAGACCATAATTGATGATAGACCT
AATACATTTCCTCTGTGTGTGTGTGTAACATTCCAAATACTTTTTTTTTCTTTTCCACTGTTTG
TAAGGTGCAACAATTTAATATTTTTAAGGGACTTTTTAAGAGTTCCTTAAGAACCAATTTAAAA
TTACTTCAGTGCAATCCTACACAGTATCAACATTAGAATTTTGATATTAGTCTTATGTTATCTT
CCATTCTATTTTTATCTGCTTTTTGCTGCTAGTTTCAAACTGCCAGTATTTTTCCTTTTGCTTT
TAAAATAGTTACAATATTTTTCATGATAGCCACAGTATTGCCACAGTTTATTATAATAAAGGGT
TTTTATTTGATTTAGCGCATTCAAAGCTTTTTTCTATCACTTTTGTGTTCAGAATATAACCTTT
GTGTGCGTGTATGTTGTGTGTGTGCATGTGTGGCGTATATGTGTGTTACAGGTTAATGCCTTCT
TGGAATTGTGTTAATGTTCTCTTGGTTTATTATGCCATCAGAATGGTAAATGAGAACACTACAA
CTGTAGTCAGCTCACAATTTTTAAATAAAGGATACCACAGTGCATGCTGTTTGTTCAATCTTTG
CAGACTTCTCTTTCTTTCCATGCTACCAGTTGTAAAGGACACAGCTATATCCTGGAAATGAAAA
ACAAACACTGTGGTGCCTAGATGTGAAGAACTGGCTTATGTGGTTGTGTTTTGCTATGGAACAG
AATGATTTAGGAAGTTCTTGTTTATATAGGTAGCCGAATTTACACATTTAGTTCAAAATTTCTC
TTGAGCATCAGCTTAGTACTATATCAATCATTCTAGAAGGATATCTTATAGAGCAGGTGTCCCC
AACCCCCGGTAAGTAGCCTGTTAGGAACCAGGTCACACATAAGGAGGTTAGCAGTGGGCAAGTG
AGCAAAGCATCATCTATATTTACAGCCGATCCCCATTGCTCGCATTACCGCCTGAGCTCTGCCT
CCTGTCAGATCAGCGGCAGCATCAGGTTCTCTCAGGAACGAACTCTATTGTGAACTGCTCATGT
GAGGGATCTAGGTTTCACGCTCCTTATGAGAATCTAACACCTGATGATCTGTCACTGTCTCCCA
TCACCCCTAGATGGGACAGTCTAGTTGCAGGAAAACAAGCTCAGGGCTTCCACTGATTCTACAT
TATGATGAGTTGTATACTTATTTTATATTATAATAAAATATATTATTACAATGTAATAATAATA
GAAATAAAATGCACAATACATGTAATGTGCTTGAATAATCCCAAAACCACCCCCTCTCCTGGTC
CATGGAAAAACTGTCTTCCATGAAAGTGGTCCCTGGTGCCAAAAAGGTTAGGGACCACTGTTAC
AGAGTATCAGGTCCTCAAGATGCTAAAATCTATATGACATTTTTAACATGTGACATTATCATCA
TCATCATCATCATCATCACTGATGATACTATTTACCAGGGCATGGTTTGAATTGGTGACTTTGG
TGCAGTTCATTATTGGCAGCCAAATGCTTTATCCATACCTTCATATTGAAGAATTTGTTATCAG
GAAACTACCAGTCCTGCTTTACAGGAAGTCTGTTATCAGATATCAGATGGCAAGTTCCCCATGT
CTTCAGATGTTCAAACAATATTGTGGATGGTCTAGAAAGAGTTTAAGACATGCTGTTAAATGTA
GGGCTAGATAATTCTCTGATTCTTTGATGTAGTCTGGAAAGAAACAATCCATTGTCCAGTTAAT
AAATATTTAGTGTTTTCATTTTTAAGACACTCACAATCCACAAATGTCCCTAACAATTTATTAT
TTTTAAAGAAAATGACTTTTTATTCCTTGCTAGTGAAAAATGTACAATTTATATGCTGCACTGA
GAAAAATAACAGATATACTTTCTTCCATTCATTTTCATCCCAAACATATAAAAAATAATCCATT
GATTGTTCCTTGCATTGCATATCTTATTAAAAGATATTTCCTACATGCAACTAATAAGACATGC
TGACTGTTGTCAGCTCTAAATTTATGTAAAGATTTTTTATTTTTGTTAAAATGTTTGAAATTTG
CTTTTTGCTCCACACCTCACCTGTTTTTGATAAATCTGTGCTAATAAGTACATAGGAGGTAATA
AATAATTGAGTTGTGAGAAACCCAATTCTCCATTTTTCAAGAAAACACAAGTAGCACTACTCTC
TTTATCCCTTGGGACTCCTTCCTGTAATTTGAAAGGGGGGGCTAATGTTGGTGGCAGTTGTTGC
TAAGGATCTCTGCACAGAGTTAGGGCTTCACTCTCGGCTTCCTGGTTAGATGGGGCCATGTGAC
TAGTCCTAGTCAGTGAGCTGTGACCAGAAGTGATGTATGTACCCTCGGGACCAGAGCATTTATG
TGCTGGTACAAGCCTCTCCATAATGACGGGCAACACATGAGATGGTGGCTGCTGCAGCCATCTG
GGTCTCTAGCCAACCCAAAATGGGGACATAACACAAGCAAGAAATAAACATTTGTTCTTATAAA
TCACCAAGATTTTGGGGTTCTCAGTGGCTGCAACATAACCTAGACTATCCTGATACAGGCTTAT
CACCATGTTCTGGATGTGTGGTGACCAGGATGGTAGCCACTACTCACAAGTGGCTATTTAAATT
TAAGTTACTTTAAAAAATAAAATAAAAAATTCAGTTCCTCAGTTGTATGAGTTACATTTCAAAT
GCTCGACAGTCACATGTACATCACTTTTCAAATTATGTGAAATTAAAACTGAAAAGCTTGTGTG
AAAGTCTAGAGAGTATGGTCTATAGTTTCATCAATTGTATTGGATAATATAGTCTCGATACTCA
A

In some embodiments, the oligonucleotide may have region of complementarity to a mutant form of MLCK1, for example as reported in Halim D. et al. “Loss-of-Function Variants in MYLK Cause Recessive Megacystis Microcolon Intestinal Hypoperistalsis Syndrome.” Am J Hum Genet. 2017 Jul. 6; 101(1):123-129; Hannuksela M. et al. “A novel variant in MYLK causes thoracic aortic dissections: genotypic and phenotypic description.” BMC Med Genet. 2016 Sep. 1; 17(1):61; or Shalata, A. et al. “Fatal thoracic aortic aneurysm and dissection in a large family with a novel MYLK gene mutation: delineation of the clinical phenotype.” Orphanet J Rare Dis. 2018 Mar. 15; 13(1):41; the contents of each of which are incorporated herein by reference in their entireties.

In some embodiments, the oligonucleotide comprises a region of complementarity to an MCLK1 mRNA sequence as set forth in SEQ ID NO: 411. In some embodiments, the region of complementarity is at least 8, at least 9, at least 10, at least 11, at least 12, at least 13, at least 14, at least 15, at least 16, at least 17, at least 19 or at least 20 nucleotides in length. In some embodiments, the region of complementarity is 8, 9, 10, 11, 12, 13, 14, 15, 16, 17, 18, 19, or 20 nucleotides in length. In some embodiments, the region of complementarity is in the range of 8 to 20, 10 to 20 or 15 to 20 nucleotides in length. In some embodiments, the region of complementarity is fully complementarity with all or a portion of its target sequence. In some embodiments, the region of complementarity includes 1, 2, 3 or more mismatches. In some embodiments, the oligonucleotide comprises a region of complementarity that is complementary (e.g., at least 85% at least 90%, at least 95%, or 100%) to an MLCK1 mRNA target sequence as set forth in SEQ ID NO: 411.

e. ACVR1 Oligonucleotides

Examples of oligonucleotides useful for targeting ACVR1 are provided in Star, G. P. et al., “ALK2 and BMPR2 knockdown and endothelin-1 production by pulmonary microvascular endothelial cells”, Microvasc Res. 2013 January; 85:46-53.; Karbiener M. et al., “MicroRNA-30c promotes human adipocyte differentiation and co-represses PAI-1 and ALK2”, RNA Biol. 2011 Sep-Oct; 8(5):850-60.; US Patent Application 2018/0087110, published Mar. 29, 2018, “Compositions and methods for xi chromosome reactivation”; US Patent Application 2009/0253132, published Oct. 8, 2009, “Mutated ACVR1for diagnosis and treatment of fibrodyplasia ossificans progressiva (FOP)”; WO 2015/152183, published Oct. 8, 2015, “Prophylactic agent and therapeutic agent for fibrodysplasia ossificans progressive”; Lowery, J. W. et al, “Allele-specific RNA Interference in FOP-Silencing the FOP gene”, GENE THERAPY, vol. 19, 2012, pages 701-702; Takahashi, M. et al. “Disease-causing allele-specific silencing against the ALK2 mutants, R206H and G356D, in fibrodysplasia ossificans progressiva” Gene Therapy (2012) 19, 781-785; Shi, S. et al. “Antisense-Oligonucleotide Mediated Exon Skipping in Activin-Receptor-Like Kinase 2: Inhibiting the Receptor That Is Overactive in Fibrodysplasia Ossificans Progressiva” Plos One, July 2013, Vol 8:7, e69096.; US Patent Application 2017/0159056, published Jun. 8, 2017, “Antisense oligonucleotides and methods of use thereof”; U.S. Pat. No. 8,859,752, issued Oct. 4, 2014, “SIRNA-based therapy of Fibrodyplasia Ossificans Progressiva (FOP)”; WO 2004/094636, published Nov. 4, 2004, entitled “Effective sirna knock-down constructs”; and Maruyama, R. and T. Yokota, “Morpholino-Mediated Exon Skipping Targeting Human ACVR1/ALK2 for Fibrodysplasia Ossificans Progressiva” Methods Mol Biol. 2018; 1828:497-502.; the contents of each of which are incorporated herein in their entireties.

In some embodiments, oligonucleotides may have a region of complementarity to a human ACVR1 sequence, for example, as provided below (Gene ID: 90; NCBI Ref. No: NM_001105.5):

(SEQ ID NO: 429)
CCTTTCCCCTGGAGATTTGAACGCTGCTTGCATGGGAGAAAAGCTACTTA
GAGAAGAAAACGTTCCACTTAGTAACAGAAGAAAAGTCTTGGTTAAAAAG
TTGTCATGAATTTGGCTTTTGGAGAGAGGCAGCAAGCCTGGAGCATTGGT
AAGCGTCACACTGCCAAAGTGAGAGCTGCTGGAGAACTCATAATCCCAGG
AACGCCTCTTCTACTCTCCGAGTACCCCAGTGACCAGAGTGAGAGAAGCT
CTGAACGAGGGCACGCGGCTTGAAGGACTGTGGGCAGATGTGACCAAGAG
CCTGCATTAAGTTGTACAATGGTAGATGGAGTGATGATTCTTCCTGTGCT
TATCATGATTGCTCTCCCCTCCCCTAGTATGGAAGATGAGAAGCCCAAGG
TCAACCCCAAACTCTACATGTGTGTGTGTGAAGGTCTCTCCTGCGGTAAT
GAGGACCACTGTGAAGGCCAGCAGTGCTTTTCCTCACTGAGCATCAACGA
TGGCTTCCACGTCTACCAGAAAGGCTGCTTCCAGGTTTATGAGCAGGGAA
AGATGACCTGTAAGACCCCGCCGTCCCCTGGCCAAGCCGTGGAGTGCTGC
CAAGGGGACTGGTGTAACAGGAACATCACGGCCCAGCTGCCCACTAAAGG
AAAATCCTTCCCTGGAACACAGAATTTCCACTTGGAGGTTGGCCTCATTA
TTCTCTCTGTAGTGTTCGCAGTATGTCTTTTAGCCTGCCTGCTGGGAGTT
GCTCTCCGAAAATTTAAAAGGCGCAACCAAGAACGCCTCAATCCCCGAGA
CGTGGAGTATGGCACTATCGAAGGGCTCATCACCACCAATGTTGGAGACA
GCACTTTAGCAGATTTATTGGATCATTCGTGTACATCAGGAAGTGGCTCT
GGTCTTCCTTTTCTGGTACAAAGAACAGTGGCTCGCCAGATTACACTGTT
GGAGTGTGTCGGGAAAGGCAGGTATGGTGAGGTGTGGAGGGGCAGCTGGC
AAGGGGAGAATGTTGCCGTGAAGATCTTCTCCTCCCGTGATGAGAAGTCA
TGGTTCAGGGAAACGGAATTGTACAACACTGTGATGCTGAGGCATGAAAA
TATCTTAGGTTTCATTGCTTCAGACATGACATCAAGACACTCCAGTACCC
AGCTGTGGTTAATTACACATTATCATGAAATGGGATCGTTGTACGACTAT
CTTCAGCTTACTACTCTGGATACAGTTAGCTGCCTTCGAATAGTGCTGTC
CATAGCTAGTGGTCTTGCACATTTGCACATAGAGATATTTGGGACCCAAG
GGAAACCAGCCATTGCCCATCGAGATTTAAAGAGCAAAAATATTCTGGTT
AAGAAGAATGGACAGTGTTGCATAGCAGATTTGGGCCTGGCAGTCATGCA
TTCCCAGAGCACCAATCAGCTTGATGTGGGGAACAATCCCCGTGTGGGCA
CCAAGCGCTACATGGCCCCCGAAGTTCTAGATGAAACCATCCAGGTGGAT
TGTTTCGATTCTTATAAAAGGGTCGATATTTGGGCCTTTGGACTTGTTTT
GTGGGAAGTGGCCAGGCGGATGGTGAGCAATGGTATAGTGGAGGATTACA
AGCCACCGTTCTACGATGTGGTTCCCAATGACCCAAGTTTTGAAGATATG
AGGAAGGTAGTCTGTGTGGATCAACAAAGGCCAAACATACCCAACAGATG
GTTCTCAGACCCGACATTAACCTCTCTGGCCAAGCTAATGAAAGAATGCT
GGTATCAAAATCCATCCGCAAGACTCACAGCACTGCGTATCAAAAAGACT
TTGACCAAAATTGATAATTCCCTCGACAAATTGAAAACTGACTGTTGACA
TTTTCATAGTGTCAAGAAGGAAGATTTGACGTTGTTGTCATTGTCCAGCT
GGGACCTAATGCTGGCCTGACTGGTTGTCAGAATGGAATCCATCTGTCTC
CCTCCCCAAATGGCTGCTTTGACAAGGCAGACGTCGTACCCAGCCATGTG
TTGGGGAGACATCAAAACCACCCTAACCTCGCTCGATGACTGTGAACTGG
GCATTTCACGAACTGTTCACACTGCAGAGACTAATGTTGGACAGACACTG
TTGCAAAGGTAGGGACTGGAGGAACACAGAGAAATCCTAAAAGAGATCTG
GGCATTAAGTCAGTGGCTTTGCATAGCTTTCACAAGTCTCCTAGACACTC
CCCACGGGAAACTCAAGGAGGTGGTGAATTTTTAATCAGCAATATTGCCT
GTGCTTCTCTTCTTTATTGCACTAGGAATTCTTTGCATTCCTTACTTGCA
CTGTTACTCTTAATTTTAAAGACCCAACTTGCCAAAATGTTGGCTGCGTA
CTCCACTGGTCTGTCTTTGGATAATAGGAATTCAATTTGGCAAAACAAAA
TGTAATGTCAGACTTTGCTGCATTTTACACATGTGCTGATGTTTACAATG
ATGCCGAACATTAGGAATTGTTTATACACAACTTTGCAAATTATTTATTA
CTTGTGCACTTAGTAGTTTTTACAAAACTGCTTTGTGCATATGTTAAAGC
TTATTTTTATGTGGTCTTATGATTTTATTACAGAAATGTTTTTAACACTA
TACTCTAAAATGGACATTTTCTTTTATTATCAGTTAAAATCACATTTTAA
GTGCTTCACATTTGTATGTGTGTAGACTGTAACTTTTTTTCAGTTCATAT
GCAGAACGTATTTAGCCATTACCCACGTGACACCACCGAATATATTACTG
ATTTAGAAGCAAAGATTTCAGTAGAATTTTAGTCCTGAACGCTACGGGGA
AAATGCATTTTCTTCAGAATTATCCATTACGTGCATTTAAACTCTGCCAG
AAAAAAATAACTATTTTGTTTTAATCTACTTTTTGTATTTAGTAGTTATT
TGTATAAATTAAATAAACTGTTTTCAAGTCAAA.

In some embodiments, oligonucleotides may have a region of complementarity to a sequence set forth as follows, which is an example mouse ACVR1 gene sequence (Gene ID 11477; NM_001110204.1)

(SEQ ID NO: 430)
CCGCCTCCCCGGGTTCAGCACCCGACCGCCGCTGGACCAGAGGAACAAAG
GAGCTGCCCCCGTGTCACCCAGCCCTTCAGTGGAAGTCTGGAAAAGGAAC
AGAGGTGATATTGCAGTGGATGAGCAGAGAGAAGCCGGCCTCTGGTGCTC
TTGAGCTGGTCTGCCCATAGGGAGCCTGCTGGGAGAAGGTACAGCTTCCG
GAAGACTCCTCCCGGAGCGCCTCTCCCATCCTCCTCTCCCTTGGAGCAGT
CAGTACCTCTCTGCTGGAGGATCTGGGCTGGGTGTGCCGGGAGCTGGCTT
TAACTGTAGCCCTGTCAGGCTTTCCCCGGACCTCGCGGAAGAGCGTCACC
AGCCCCCACGGCTTTCCAACACATCACCTCTTTTCATGCCGTTTGGCACA
GATCGAATCTACAGGGATGAATGGATCCAGGGTCTGGTTTTAAGTTCTAT
GGTAGTCGTCCAAGGAGCCATTGGTATTCATCTAACGCAAACGATCAAGT
TACATTCTGAAAGTAACATCCCACCAGAAACCCTCCAGCAGCAGTCACGT
CTGTGTAAAGCCAAGCCCTGGCATGCGCACTGCCAGGTCAGAGTGTGGTG
GTACACGTGTTTAACAGGTCATTTGTCAACTGAAGGAAAGACCCCGGCTT
GACTTACCTGTTATACAATGGTCGATGGAGTAATGATCCTTCCTGTGCTA
ATGATGATGGCTTTCCCTTCCCCGAGTGTGGAAGATGAGAAGCCCAAGGT
CAACCAGAAACTTTACATGTGTGTGTGTGAGGGCCTCTCCTGCGGGAACG
AGGACCACTGTGAAGGCCAGCAGTGTTTTTCTTCTCTGAGCATCAACGAT
GGCTTCCACGTCTACCAGAAGGGCTGCTTTCAGGTTTATGAGCAGGGGAA
GATGACGTGTAAGACCCCGCCGTCACCTGGCCAGGCTGTGGAGTGCTGCC
AAGGGGACTGGTGTAACAGGAACATCACGGCCCAGCTGCCCACTAAAGGG
AAGTCCTTCCCCGGAACACAGAATTTCCACCTGGAAGTTGGCCTTATCAT
CCTCTCGGTGGTGTTTGCAGTATGTCTTTTAGCTTGCATCCTTGGAGTTG
CTCTCAGGAAGTTTAAGAGACGCAATCAAGAGCGCCTGAACCCCAGAGAC
GTGGAGTATGGTACCATTGAAGGGCTCATCACCACCAATGTGGGAGACAG
CACTCTAGCGGAACTACTAGATCACTCGTGTACATCAGGAAGTGGCTCCG
GTCTTCCTTTCCTGGTACAGAGAACGGTGGCTCGCCAGATAACCCTGTTG
GAGTGTGTCGGGAAGGGCCGGTATGGAGAAGTATGGAGGGGCAGCTGGCA
AGGCGAAAATGTCGCTGTGAAGATCTTCTCCTCCCGAGACGAGAAGTCAT
GGTTCAGGGAGACGGAATTGTACAACACTGTGATGTTGAGGCATGAAAAT
ATCTTAGGTTTCATCGCTTCAGACATGACCTCCAGACACTCCAGTACCCA
GCTGTGGCTCATCACACATTACCATGAAATGGGATCGTTGTATGACTACC
TTCAGCTCACTACTCTGGATACGGTTAGCTGCCTTCGGATTGTACTGTCC
ATAGCCAGCGGCCTTGCCCATTTGCACATAGAGATATTTGGGACCCAAGG
GAAGTCCGCCATTGCCCATCGAGATCTGAAGAGCAAAAACATCCTGGTGA
AGAAGAATGGACAGTGCTGCATAGCAGATTTGGGCCTGGCAGTCATGCAT
TCCCAGAGCACAAACCAGCTTGATGTGGGAAACAACCCCCGTGTGGGGAC
CAAGCGCTACATGGCTCCGGAAGTGCTCGATGAAACCATCCAAGTGGATT
GCTTTGATTCTTATAAGAGGGTCGATATTTGGGCCTTTGGCCTTGTTCTG
TGGGAAGTGGCCAGGCGAATGGTGAGCAATGGTATAGTGGAAGATTACAA
GCCACCATTCTATGATGTGGTTCCCAATGACCCAAGTTTTGAAGATATGA
GGAAAGTTGTCTGTGTGGATCAACAGAGGCCAAACATACCTAACAGATGG
TTCTCAGACCCGACATTAACTTCTCTGGCGAAGCTGATGAAAGAGTGCTG
GTATCAGAACCCATCCGCAAGACTCACAGCTCTACGTATCAAAAAGACTT
TGACCAAAATCGATAATTCCCTAGACAAATTAAAAACTGACTGTTGACCT
TGTCACCGGTGTCAAGAAGGAGAGTCAATGCTGTCCTTGTCCAGCTGGGA
CCTAATGCTGGCCTGACTGGTTGTCAGAACAGAATCCATCTGACCCCCTT
CCCGAAGTGGCTGCTTTGACGGAAGCAGATGTCTCTTCCCAGCCATGTTC
CAGGGGGAGACACCAAAACCACCCTAACCTCGCTCAAAAACTGTGACTCG
AGCCCTCGATGAACTGTTCACACCACAAAGACTTAACGGTGGGCAGGTCT
GGTGGCAAGGGGGAGGGAAGTGGAGGAACCCGGAAAGATCCTGCAGGCGA
TCTGGGCATTAAGACAGTGGCTCTCTGCGTATCTTTCGCGGGTCTCCTAG
ACACTCCCCACGGGAAGCTCAAGGAGGCGGTGAATTCGTAATCAGCAATA
TCGGCTGCATCTACTCTTCGTTGCACTAGGAATTCTGTGCATTCCTTACT
TGCACTGTGGCCCTTAATCTTAAAGACCCAACTTGCCAAAACATTGGCTG
CGTACTCCACTGGCCTGTCTCTGGATAATAGGAATTCAATCTGGCAACAC
AAAAATGTACCGTTGGACTCTGCTGCATTTTACACACGTGCTGATGTTTA
CAAGGATGCGAACATTAGGAATTGTTTAGACACAACTTTGCAAATTATTT
ATTACTGGTGCACTTAGCGGTTTGTTTGAAACCGCCTCGTGCATATGTTA
AAGCTTATTTTTATGTGGTCTTATGATTTTATTACCGAAATGTTTTTAAC
ACCCAACTCTGAAACGGACATTTTCTTTTATTATCAGTTAAATTCACATT
TAAGTGCTTCACATTTTTTTTTTTAAATGTGTGTAGACTGTAACTTTCTT
TTCAGTTCGTATGCAGAACATATTTAGCCATTACCCATGCAACACCACCC
GATATATTACTGATTTAGAAGCAAAGATTTCAGTAGAATTTTAGTCCCAA
ACGCTGTGGGGGGAAATGCATCTTCTTCGGAATTATCCATTACGTGCATT
TAAACTCTGCCAGAAAAAAAAATAACTATTTTGTTTTAATCTACTTTTTG
TATTTAGTAGTTATTTGTATAAATTAAATAAACTGTTTTCAAGTCAAAAA
AAAAAAAAAAAA

In some embodiments, an oligonucleotide comprises a region of complementarity to an ACVR1 sequence as set forth in SEQ ID NO: 429 or SEQ ID NO: 430. In some embodiments, the oligonucleotide comprises a region of complementarity that is at least 80%, 85%, 90%, 95%, 96%, 97%, 98%, 99%, or 100% complementary to an ACVR1 sequence as set forth in SEQ ID NO: 429 or SEQ ID NO: 430. In some embodiments, the oligonucleotide comprises a sequence that has at least 10, 11, 12, 13, 14, 15, 16, 17, 18, or 19 consecutive nucleotides that are perfectly complementary to an ACVR1 sequence as set forth in SEQ ID NO: 429 or SEQ ID NO: 430. In some embodiments, an oligonucleotide may comprise a sequence that targets (e.g., is complementary to) an RNA version (i.e., wherein the T's are replaced with U's) of an ACVR1 sequence as set forth in SEQ ID NO: 429 or SEQ ID NO: 430. In some embodiments, the oligonucleotide comprises a sequence that is complementary (e.g., at least 80%, 85%, 90%, 95%, 96%, 97%, 98%, 99%, or 100% complementary) to an RNA version of an ACVR1 sequence as set forth in SEQ ID NO: 429 or SEQ ID NO: 430. In some embodiments, the oligonucleotide comprises a sequence that has at least 10, 11, 12, 13, 14, 15, 16, 17, 18, or 19 consecutive nucleotides that are perfectly complementary to an RNA version of an ACVR1 sequence as set forth in SEQ ID NO: 429 or SEQ ID NO: 430.

In some embodiments, an ACVR1-targeting oligonucleotide comprises an antisense strand that comprises at least 10, 11, 12, 13, 14, 15, 16, 17, 18, or 19 consecutive nucleotides of a sequence comprising any one of SEQ ID NOs: 479-502. In some embodiments, an ACVR1-targeting oligonucleotide comprises an antisense strand that comprises any one of SEQ ID NOs: 479-502. In some embodiments, an ACVR1-targeting oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 479-502.

In some embodiments, an ACVR1-targeting oligonucleotide comprises an antisense strand that targets an ACVR1 sequence comprising any one of SEQ ID NOs: 431-478. In some embodiments, an oligonucleotide comprises an antisense strand comprising at least 10, 11, 12, 13, 14, 15, 16, 17, 18, or 19 nucleotides (e.g., consecutive nucleotides) that are complementary to an ACVR1 sequence comprising any one of SEQ ID NOs: 431-478. In some embodiments, an ACVR1-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NOs: 431-478.

In some embodiments, an ACVR1-targeting oligonucleotide comprises an antisense strand comprises a region of complementarity to a target sequence as set forth in any one of SEQ ID NOs: 431-478. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, an ACVR1-targeting oligonucleotide further comprises a sense strand that hybridizes to the antisense strand to form a double stranded siRNA. In some embodiments, the ACVR1-targeting oligonucleotide comprises an antisense strand that comprises the nucleotide sequence of any one of SEQ ID NOs: 479-502. In some embodiments, the ACVR1-targeting oligonucleotide further comprises a sense strand that comprises the nucleotide sequence of any one of SEQ ID NOs: 455-478.

In some embodiments, the ACVR1-targeing oligonucleotide is a double stranded oligonucleotide (e.g., an siRNA) comprising an antisense strand that comprises the nucleotide sequence of any one of SEQ ID NOs: 479-502 and a sense strand that hybridizes to the antisense strand and comprises the nucleotide sequence of any one of SEQ ID NOs: 455-478, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the ACVR1-targeing oligonucleotide is a double stranded oligonucleotide (e.g., an siRNA) comprising an antisense strand that comprises the nucleotide sequence of any one of SEQ ID NOs: 479-502 and a sense strand that hybridizes to the antisense strand and comprises the nucleotide sequence of any one of SEQ ID NOs: 455-478, wherein the each nucleoside in the antisense strand and/or (e.g., and) each nucleoside in the sense strand is a 2′-modified nucleoside selected from 2′-O-Me and 2′-F modified nucleosides.

In some embodiments, the ACVR1-targeing oligonucleotide is a double stranded oligonucleotide (e.g., an siRNA) comprising an antisense strand that comprises the nucleotide sequence of any one of SEQ ID NOs: 479-502 and a sense strand that hybridizes to the antisense strand and comprises the nucleotide sequence of any one of SEQ ID NOs: 455-478, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the ACVR1-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the ACVR1-targeing oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the ACVR1-targeing oligonucleotide is selected from the modified version of SEQ ID NOs: 479-502 listed in Table 19. In some embodiments, the sense strand of the ACVR1-targeing oligonucleotide is selected from the modified version of SEQ ID NOs: 455-478 listed in Table 19. In some embodiments, the ACVR1-targeing oligonucleotide is a siRNA selected from the siRNAs listed in Table 19.

TABLE 17
ACVR1 Target Sequences

| Corresponding | | |
|---|---|---|
| nucleotides | | |
| of Sequence | | |
| NM_001105.5 | | SEQ |
| (SEQ ID | ACVR1 Target | ID |
| NO: 429) | Sequence | NO: |
| 877-895 | TCGTGTACATCAGGAAGTG | 431 |
| 878-896 | CGTGTACATCAGGAAGTGG | 432 |
| 1380-1398 | TTTGGGCCTGGCAGTCATG | 433 |
| 1573-1591 | GTGAGCAATGGTATAGTGG | 434 |
| 1486-1704 | TGAGCAATGGTATAGTGGA | 435 |
| 1574-1592 | CCCAAGTTTTGAAGATATG | 436 |
| 1633-1651 | CCAAGTTTTGAAGATATGA | 437 |
| 1693-1711 | AACAGATGGTTCTCAGACC | 438 |
| 1696-1714 | AGATGGTTCTCAGACCCGA | 439 |
| 1904-1922 | ACCTAATGCTGGCCTGACT | 440 |
| 2484-2502 | TTGCAAATTATTTATTACT | 441 |
| 2617-2635 | TTTTCTTTTATTATCAGTT | 442 |
| 2667-2685 | TGTGTGTAGACTGTAACTT | 443 |
| 2742-2760 | ATATTACTGATTTAGAAGC | 444 |
| 2752-2770 | TTTAGAAGCAAAGATTTCA | 445 |
| 2757-2775 | AAGCAAAGATTTCAGTAGA | 446 |
| 2758-2776 | AGCAAAGATTTCAGTAGAA | 447 |
| 2759-2777 | GCAAAGATTTCAGTAGAAT | 448 |
| 2760-2778 | CAAAGATTTCAGTAGAATT | 449 |
| 2834-2852 | CATTTAAACTCTGCCAGAA | 450 |
| 2859-2877 | AACTATTTTGTTTTAATCT | 451 |
| 2882-2900 | TTTGTATTTAGTAGTTATT | 452 |
| 2912-2930 | AATAAACTGTTTTCAAGTC | 453 |
| 2913-2931 | ATAAACTGTTTTCAAGTCA | 454 |

* The target sequences contain Ts, but binding to RNA and/or DNA is contemplated.

In some embodiments, an oligonucleotide may comprise or consist of any sequence as provided in Table 18.

TABLE 18
Oligonucleotide sequences for targeting ACVR1

| Passenger | SEQ | | SEQ |
|---|---|---|---|
| strand (RNA)/ | ID | Guide strand (RNA)/ | ID |
| Sense strand | NO: | Antisense strand | NO: |
| AUUCGUGUACAUCAGGAAGUG | 455 | CACUUCCUGAUGUACACGAAUGA | 479 |
| UUCGUGUACAUCAGGAAGUGG | 456 | CCACUUCCUGAUGUACACGAAUG | 480 |
| GAUUUGGGCCUGGCAGUCAUG | 457 | CAUGACUGCCAGGCCCAAAUCUG | 481 |
| UGGUGAGCAAUGGUAUAGUGG | 458 | CCACUAUACCAUUGCUCACCAUC | 482 |
| GGUGAGCAAUGGUAUAGUGGA | 459 | UCCACUAUACCAUUGCUCACCAU | 483 |
| GACCCAAGUUUUGAAGAUAUG | 460 | CAUAUCUUCAAAACUUGGGUCAU | 484 |
| ACCCAAGUUUUGAAGAUAUGA | 461 | UCAUAUCUUCAAAACUUGGGUCA | 485 |
| CCAACAGAUGGUUCUCAGACC | 462 | GGUCUGAGAACCAUCUGUUGGGU | 486 |
| ACAGAUGGUUCUCAGACCCGA | 463 | UCGGGUCUGAGAACCAUCUGUUG | 487 |
| GGACCUAAUGCUGGCCUGACU | 464 | AGUCAGGCCAGCAUUAGGUCCCA | 488 |
| CUUUGCAAAUUAUUUAUUACU | 465 | AGUAAUAAAUAAUUUGCAAAGUU | 489 |
| CAUUUUCUUUUAUUAUCAGUU | 466 | AACUGAUAAUAAAAGAAAAUGUC | 490 |
| UAUGUGUGUAGACUGUAACUU | 467 | AAGUUACAGUCUACACACAUACA | 491 |
| AUAUAUUACUGAUUUAGAAGC | 468 | GCUUCUAAAUCAGUAAUAUAUUC | 492 |
| GAUUUAGAAGCAAAGAUUUCA | 469 | UGAAAUCUUUGCUUCUAAAUCAG | 493 |
| AGAAGCAAAGAUUUCAGUAGA | 470 | UCUACUGAAAUCUUUGCUUCUAA | 494 |
| GAAGCAAAGAUUUCAGUAGAA | 471 | UUCUACUGAAAUCUUUGCUUCUA | 495 |
| AAGCAAAGAUUUCAGUAGAAU | 472 | AUUCUACUGAAAUCUUUGCUUCU | 496 |
| AGCAAAGAUUUCAGUAGAAUU | 473 | AAUUCUACUGAAAUCUUUGCUUC | 497 |
| UGCAUUUAAACUCUGCCAGAA | 474 | UUCUGGCAGAGUUUAAAUGCACG | 498 |
| AUAACUAUUUUGUUUUAAUCU | 475 | AGAUUAAAACAAAAUAGUUAUUU | 499 |
| UUUUUGUAUUUAGUAGUUAUU | 476 | AAUAACUACUAAAUACAAAAAGU | 500 |
| UAAAUAAACUGUUUUCAAGUC | 477 | GACUUGAAAACAGUUUAUUUAAU | 501 |
| AAAUAAACUGUUUUCAAGUCA | 478 | UGACUUGAAAACAGUUUAUUUAA | 502 |

In some embodiments, an oligonucleotide is a modified oligonucleotide as provided in Table 19, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 19
Modified Oligonucleotides for targeting ACVR1

| | Modified | | Modified | |
|---|---|---|---|---|
| | Passenger strand/ | SEQ ID | Guide strand/ | SEQ ID |
| siRNA # | Sense strand | NO: | Antisense strand | NO: |
| hsACVR1-24 | mAmUfUmCfGmUfGmUfAmCf | 455 | fCfAmCfUmUfCmCfUmGfAm | 479 |
| | AmUfCmAfGmGfAmAfGmUfG | | UfGmUfAmCfAmCfGmAfAm | |
| | | | U*fG*mA | |
| hsACVR1-14 | mUmUfCmGfUmGfUmAfCmAf | 456 | fCfCmAfCmUfUmCfCmUfGm | 480 |
| | UmCfAmGfGmAfAmGfUmGfG | | AfUmGfUmAfCmAfCmGfAm | |
| | | | A*fU*mG | |
| hsACVR1-22 | mGmAfUmUfUmGfGmGfCmCf | 457 | fCfAmUfGmAfCmUfGmCfCm | 481 |
| | UmGfGmCfAmGfUmCfAmUfG | | AfGmGfCmCfCmAfAmAfUmC | |
| | | | *fU*mG | |
| hsACVR1-23 | mUmGfGmUfGmAfGmCfAmAf | 458 | fCfCmAfCmUfAmUfAmCfCm | 482 |
| | UmGfGmUfAmUfAmGfUmGfG | | AfUmUfGmCfUmCfAmCfCmA | |
| | | | *fU*mC | |
| hsACVR1-18 | mGmGfUmGfAmGfCmAfAmUf | 459 | fUfCmCfAmCfUmAfUmAfCm | 483 |
| | GmGfUmAfUmAfGmUfGmGfA | | CfAmUfUmGfCmUfCmAfCmC | |
| | | | *fA*mU | |
| hsACVR1-13 | mGmAfCmCfCmAfAmGfUmUf | 460 | fCfAmUfAmUfCmUfUmCfAm | 484 |
| | UmUfGmAfAmGfAmUfAmUfG | | AfAmAfCmUfUmGfGmGfUm | |
| | | | C*fA*mU | |
| hsACVR1-7 | mAmCfCmCfAmAfGmUfUmUf | 461 | fUfCmAfUmAfUmCfUmUfCm | 485 |
| | UmGfAmAfGmAfUmAfUmGfA | | AfAmAfAmCfUmUfGmGfGm | |
| | | | U*fC*mA | |
| hsACVR1-20 | mCmCfAmAfCmAfGmAfUmGf | 462 | fGfGmUfCmUfGmAfGmAfAm | 486 |
| | GmUfUmCfUmCfAmGfAmCfC | | CfCmAfUmCfUmGfUmUfGm | |
| | | | G*fG*mU | |
| hsACVR1-11 | mAmCfAmGfAmUfGmGfUmUf | 463 | fUfCmGfGmGfUmCfUmGfAm | 487 |
| | CmUfCmAfGmAfCmCfCmGfA | | GfAmAfCmCfAmUfCmUfGm | |
| | | | U*fU*mG | |
| hsACVR1-16 | mGmGfAmCfCmUfAmAfUmGf | 464 | fAfGmUfCmAfGmGfCmCfAm | 488 |
| | CmUfGmGfCmCfUmGfAmCfU | | GfCmAfUmUfAmGfGmUfCm | |
| | | | C+fC*mA | |
| hsACVR1-6 | mCmUfUmUfGmCfAmAfAmUf | 465 | fAfGmUfAmAfUmAfAmAfUm | 489 |
| | UmAfUmUfUmAfUmUfAmCfU | | AfAmUfUmUfGmCfAmAfAm | |
| | | | G*fU*mU | |
| hsACVR1-8 | mCmAfUmUfUmUfCmUfUmUf | 466 | fAfAmCfUmGfAmUfAmAfUm | 490 |
| | UmAfUmUfAmUfCmAfGmUfU | | AfAmAfAmGfAmAfAmAfUm | |
| | | | G*fU*mC | |
| hsACVR1-15 | mUmAfUmGfUmGfUmGfUmAf | 467 | fAfAmGfUmUfAmCfAmGfUm | 491 |
| | GmAfCmUfGmUfAmAfCmUfU | | CfUmAfCmAfCmAfCmAfUmA | |
| | | | *fC*mA | |
| hsACVR1-21 | mAmUfAmUfAmUfUmAfCmUf | 468 | fGfCmUfUmCfUmAfAmAfUm | 492 |
| | GmAfUmUfUmAfGmAfAmGfC | | CfAmGfUmAfAmUfAmUfAm | |
| | | | U*fU*mC | |
| hsACVR1-5 | mGmAfUmUfUmAfGmAfAmGf | 469 | fUfGmAfAmAfUmCfUmUfUm | 493 |
| | CmAfAmAfGmAfUmUfUmCfA | | GfCmUfUmCfUmAfAmAfUm | |
| | | | C*fA*mG | |
| hsACVR1-10 | mAmGfAmAfGmCfAmAfAmGf | 470 | fUfCmUfAmCfUmGfAmAfAm | 494 |
| | AmUfUmUfCmAfGmUfAmGfA | | UfCmUfUmUfGmCfUmUfCm | |
| | | | U*fA*mA | |
| hsACVR1-4 | mGmAfAmGfCmAfAmAfGmAf | 471 | fUfUmCfUmAfCmUfGmAfAm | 495 |
| | UmUfUmCfAmGfUmAfGmAfA | | AfUmCfUmUfUmGfCmUfUm | |
| | | | C*fU*mA | |
| hsACVR1-3 | mAmAfGmCfAmAfAmGfAmUf | 472 | fAfUmUfCmUfAmCfUmGfAm | 496 |
| | UmUfCmAfGmUfAmGfAmAfU | | AfAmUfCmUfUmUfGmCfUm | |
| | | | U*fC*mU | |
| hsACVR1-1 | mAmGfCmAfAmAfGmAfUmUf | 473 | fAfAmUfUmCfUmAfCmUfGm | 497 |
| | UmCfAmGfUmAfGmAfAmUfU | | AfAmAfUmCfUmUfUmGfCm | |
| | | | U*fU*mC | |
| hsACVR1-17 | mUmGfCmAfUmUfUmAfAmAf | 474 | fUfUmCfUmGfGmCfAmGfAm | 498 |
| | CmUfCmUfGmCfCmAfGmAfA | | GfUmUfUmAfAmAfUmGfCm | |
| | | | A*fC*mG | |
| hsACVR1-12 | mAmUfAmAfCmUfAmUfUmUf | 475 | fAfGmAfUmUfAmAfAmAfCm | 499 |
| | UmGfUmUfUmUfAmAfUmCfU | | AfAmAfAmUfAmGfUmUfAm | |
| | | | U*fU*mU | |
| hsACVR1-2 | mUmUfUmUfUmGfUmAfUmUf | 476 | fAfAmUfAmAfCmUfAmCfUm | 500 |
| | UmAfGmUfAmGfUmUfAmUfU | | AfAmAfUmAfCmAfAmAfAm | |
| | | | A*fG*mU | |
| hsACVR1-19 | mUmAfAmAfUmAfAmAfCmUf | 477 | fGfAmCfUmUfGmAfAmAfAm | 501 |
| | GmUfUmUfUmCfAmAfGmUfC | | CfAmGfUmUfUmAfUmUfUm | |
| | | | A*fA*mU | |
| hsACVR1-9 | mAmAfAmUfAmAfAmCfUmGf | 478 | fUfGmAfCmUfUmGfAmAfAm | 502 |
| | UmUfUmUfCmAfAmGfUmCfA | | AfCmAfGmUfUmUfAmUfUm | |
| | | | U*fA*mA | |

f. FBXO32 Oligonucleotides

Examples of oligonucleotides useful for targeting FBXO32 are provided in Cong et al., Hum Gene Ther. 2011 March; 22(3):313-24; Castillero et al., Metabolism. 2013; Oct; 62(10):1495-502; Wada et al., Nature Precedings 2008; Lagirand-Cantaloube et al., PLoS One. 2009; 4(3):e4973; U.S. Pat. No. 8,097,596, entitled, “COMPOSITIONS AND METHODS FOR THE TREATMENT OF MUSCLE WASTING,” ISSUED ON Jan. 17, 2012; WO2019139351, entitled, “PHARMACEUTICAL COMPOSITION FOR PREVENTING OR TREATING MUSCULAR DISEASE OR CACHEXIA COMPRISING, AS ACTIVE INGREDIENT, MIRNA LOCATED IN DLK1-DIO3 CLUSTER OR VARIANT THEREOF,” which was published on Jul. 18, 2019; WO2008156561, entitled, “METHODS AND COMPOSITIONS FOR THE TREATMENT AND DIAGNOSIS OF STATIN-INDUCED MYOPATHY,” which was published on Dec. 24, 2008; and WO2019113393, entitled, “COMPOSITIONS AND METHODS OF TREATING MUSCLE ATROPHY AND MYOTONIC DYSTROPHY,” which was published on Jun. 13, 2019, the contents of each of which are incorporated herein in their entireties. In some embodiments, the oligonucleotide is a CRISPR guide RNA targeting FBXO32.

In some embodiments, oligonucleotides may have a region of complementarity to a human FBXO32 sequence, for example, as provided below (Gene ID: 114907; NCBI Ref. No: NM_058229.4):

(SEQ ID NO: 505)
AGCACTCCCGGAGCCTGCAACGCTTGAGATCCTCTCCGCGCCCGCCACCCCGCAGGGTGCCCC
GCGCCGTTCCCGCCGCCCCGCCGCCCCCGTCGCGGGCCCCTGCACCCCGAGCATCCGCCCCGG
GTGGCACGTCCCCGAGCCCACCAGGCCGGCCCCGTCTCCCCATCCGTCTAGTCCGCTCGCGGT
GCCATGCCATTCCTCGGGCAGGACTGGCGGTCCCCCGGGCAGAACTGGGTGAAGACGGCCGAC
GGCTGGAAGCGCTTCCTGGATGAGAAGAGCGGCAGTTTCGTGAGCGACCTCAGCAGTTACTGC
AACAAGGAGGTATACAATAAGGAGAATCTTTTCAACAGCCTGAACTATGATGTTGCAGCCAAG
AAGAGAAAGAAGGACATGCTGAATAGCAAAACCAAAACTCAGTATTTCCACCAAGAAAAATGG
ATCTATGTTCACAAAGGAAGTACTAAAGAGCGCCATGGATATTGCACCCTGGGGGAAGCTTTC
AACAGACTGGACTTCTCAACTGCCATTCTGGATTCCAGAAGATTTAACTACGTGGTCCGGCTG
TTGGAGCTGATAGCAAAGTCACAGCTCACATCCCTGAGTGGCATCGCCCAAAAGAACTTCATG
AATATTTTGGAAAAAGTGGTACTGAAAGTCCTTGAAGACCAGCAAAACATTAGACTAATAAGG
GAACTACTCCAGACCCTCTACACATCCTTATGTACACTGGTCCAAAGAGTCGGCAAGTCTGTG
CTGGTCGGGAACATTAACATGTGGGTGTATCGGATGGAGACGATTCTCCACTGGCAGCAGCAG
CTGAACAACATTCAGATCACCAGGCCTGCCTTCAAAGGCCTCACCTTCACTGACCTGCCTTTG
TGCCTACAACTGAACATCATGCAGAGGCTGAGCGACGGGCGGGACCTGGTCAGCCTGGGCCAG
GCTGCCCCCGACCTGCACGTGCTCAGCGAAGACCGGCTGCTGTGGAAGAAACTCTGCCAGTAC
CACTTCTCCGAGCGGCAGATCCGCAAACGATTAATTCTGTCAGACAAAGGGCAGCTGGATTGG
AAGAAGATGTATTTCAAACTTGTCCGATGTTACCCAAGGAAAGAGCAGTATGGAGATACCCTT
CAGCTCTGCAAACACTGTCACATCCTTTCCTGGAAGGGCACTGACCATCCGTGCACTGCCAAT
AACCCAGAGAGCTGCTCCGTTTCACTTTCACCCCAGGACTTTATCAACTTGTTCAAGTTCTGA
ATCCCAGCACATGACAACACTTCAGAAGGGTCCCCCTGCTGACTGGAGAGCTGGGAATATGGC
ATTTGGACACTTCATTTGTAAATAGTGTACATTTTAAACATTGGCTCGAAACTTCAGAGATAA
GTCATGGAGAGGACATTGGAGGGGAGAAATGCAGTTGCTGACTGGGAATTTAAGAATGTGAAC
TTCTCACTAGAATTGGTATGGAAAAGCAAAATACTGTAAATAAACTTTTTTTCTAACAATTTG
CCAGCAAGACTATAAGGGCAATAATTCTATTTCAGCGGTGAAAATGGAGTCCTCTTAATGGTC
ACAGAAACTCTCTTATAGTTCCCTAGGAAGAAAAAGGCAAAACTCAAATACAAAATAGGACGC
TTTGTTTACAATGTGAAAATTTGTTTAGAAAAGAAAAAATGAAGAAGGAAAACTATATAGGAG
AAATTCCTGGGCTTTGGGGTTTGATCTGGTGTTTGTTTTGAGAAGGCGAAGGAAGCGCCACCC
ATCCTAAACCTGCATGGGCACAGAGCCTTACCCTACAGAGATACCACACAATGGTCTACCTCT
AAAAGCGTAGAGTGCCCTCTCTGACAACGCATGTGATCCAGTAGGCAGTGTCTGTTTTTCTCG
TAAGTGTTTTCATCACTATAAAAAGTGATTTAAAATGGAAAGGGATATTGGAGGCAATCCTCT
GGTTTCACACTTCCTTTTCAGGCAAACAGGGCCCTCCTTAGAGTTATCTGTTCACCAGAACAA
CCCAAAAGCACATGTTCCTTTTTTCCTGTCATGTTGTAGCTCATTTGGGGGAAAAGAAGGCCA
ACTTAAAACAGTCCCAGGGGAGGATTGCTTGAGCCCAGGAGTCTGAGGTTTGCAGTGAGCTAT
GATTGTGCCACTGCACTCCAGCCTGTCTCCAAAACAAAACAAAACAAAACAAAAGCCGTCCCA
GAGGAAGTCCTGCTGCAGAAAAGATGATAGAATTGGTCACCCAATCAATAGCTGTGAAGACTT
CCTTATTCCTGCTGAACTTCCTTCCTTGGATATCTTCATGGATAAGCCTGTGTCTGTGGACAG
AGTGGGGCCCAAGTGCTGAGGCTCGAAAGACAGGTTTTACAATCAGCCAATTAAACATCTTAC
CAGTTGCTGTACCCCTTGCCAAGGGGATGAGCTGAGGAGCTTCTTTTCTTTAGGTCCAACATC
AAAATGAGTCCATCTGAGCTAGGGCTTTTTGCAAAGGTGATTAAAAATCAAGGCTGGAGTTCC
AGCCAAATAGGGAGGAGAGGTACCACAAGTTCATCTTAAACTTGCTTCCGGGCTGGGTAGTTA
AAACAGGAAGATCCCAAGGGGATCTTACGAGGCAAATATCAGCAGGTAACTGTGGAAGAAAGG
AAATCTCAGGACCTAATGAGTTCGTTGTAAAATATTCCTGGGAATAGATGGGGGATCTACTCC
CAGTTTTTTACTTTTTACAGAATATTGTTGCTTTCCTACAACAATGTACATATATTTGCAGTT
GTATATGCTTTTTTTTTTTCCAAATAAACTTGTCACCCTGCATGCCCTTGGCAAATAAGTGAA
GCAGAAATAGGAACACAGTCCACATTCAAGTTGAGGAACAGTGTATCTTTAAGAGCTGAGCTT
TGGGTGACCTGGAAAGGGGGAAAGATGGCTAAGCATGGAGAGAAACGAGGCAAGAGACAAGCT
ATGATACAACACCGCTTCAGCCCCTGCCCTCAATAGCACACAACCCACATATCAGCTTTCTGA
AGAGAAGGAACCTACTGTTTAGTGCTCCTCACTTTGCAATGTTTGTGCTACGCCAGAATTTCT
CCAGTTTTTTTCATTATCATCCCCCTGAGAAAAAAATTACATTGAATTTAAATTTTCCCTAAT
AAGAGAAATTAAATATGAAAGAATAGGATTTTGTTGGGTAAGATTGAGCTTTGGAAGGTCACG
AACCATTATTCTATCTAAGGTGTGTGTTTTGTTTTGTTTTTTTTTGTTTTTTTTTTGCAATCC
CTCTCCCCCTGAACCAATTTCACATTGGGAATGCAGGACCTAGACTGCTGAATAAAAAGCTAC
TTCTTCTATAATTGTCAGGTTCTCTCCAATTTCTCAGCTTCCTCAAAGACATGGGGTGGAGTT
GGGTGTGCCATCACTGAGAGAGCTCTGGAACTTTTGACTCTTAGTGACATTTTTAGATTTAGG
GGTTCATGGCCTTCCACATGTTGCCACCAACTGGTGATCTCTGCCCCTTCATGCTGATCAAGA
AAGTAGAAACTCCTCGTCGCCTTCAGGTTTGCAGTCGCAGAAACATTGCCTGCTGTGGACTGT
CAGCACAAAACTGGGACACTGGTGTCATTTAGACTGTCAGCAGTGCACATGATTGTACGATAG
ACTCCAGGCAACCATGTGCATCTGTGCAAGATGACCTCTGCCCCAAGAGAAGGGTTACGGTCT
AATTAAATGTTTACCAAGATGGTACCAGTGGGCTCTCCCCGTGTCCTTTGGTGTTATTGGAGC
TGGTGTATGACAGACTCAAGGAAATTTTTTAAGGAAAATGAAGAAGAAATCAACCTTTATGGT
TCTCTTTCATTGGAAGAGGAGAATAAGGAAAGAAATGGCAGGTAGAGAGGGAGGGGGAAAGGA
ATAGAAATGGCATGTCTTTGATGCTGTGGCTTGTGTGGGGACAATGGGAAGAGCACAGCAGGC
ACAACATATCTGTGTTAGTGCCACGTGGTATCTGTTAAGTATGGCCAGAGCCTCACATATAAG
TGAAGAGAGTTAAGACAATTCTTGCTCTTTGAACAATAATAGTCTATAGAATTTCTATTGGCA
AACCATCCCAGACAACCTGACCTATCAAACAAAAGCAAATAATCCCTGTCTCAGAACTGCTGG
TCTAAAAGCTAGAAGGGGCATGATAATGGAAATTGCTAGAAAAGAGAGAATGCTCTACTCTCT
TTCCTGTGCCTACCTACCCCCATCCTAAACCCTGTAAAACAGAATTTCAAAATAGATGTCAAA
TATGAAGTAATTCAGACTTCCAAAGAAGGAAAGAGTTCTGCCCAGGGCAGTATGAGCAAATCC
ACAGGGATGTTAAGATTTGGTCCAACTCAAAGGTTTATGGGCAGTGAGCCTAGAGTCTCTTGA
GAGTAAACCCTTGCATTTGGGACAAGGAGAATATGTGAAGTTCAGGAGTGCTCACACTAGAGC
AAGATCCAGAAAAAAAAAAATCCAATGGCATTTTAAACTAGATTGCATTATCACTCAATGCTG
TTACTTTGAGCAGACAAATCAGTTGAATGGGAGGGCAAATGGCAGAAATGAACAAAAGCTATT
AGGTGAAAGCATCCCCAATGTATCAGTTGTGAGATGATTTTTGTTTAATGATGATCAGGTTTA
CATTGAAGTGGCTTGGAAGACGTATTTCAGAGGGACTGGGTTTGTACTGCAAAACTCTGAACA
CTAGAGCAGGTTCTACTAGCACTTGGGCAGTAAGGTAGCGGGTGTGTATTATGCTATTGCCAT
AGTTCTCGTCTTTGTGGATTCACATACCCTTTCCCATGAGGAGCTTGCTACTAACAGCCTCCT
GTTTCTGTTGTTTTTATATGGGAGAAGAGAAAGAGCTTGGAATTTCAATTGTCTAAACAATTG
GTATGATTTACAAGAAGGCAAACCATTCAGGAGATGTTCAACAGTGTATCATTCTTAGCATTC
AATACAATGTTTATTATAAAATATACACCTGAGTTTATGTTTTTCTGCCAGGCTGAAGGGCAA
TAATGTCTTGCTATGCAAACACTCTATTTTATGTGTGAATTTTTTTAACTGTAATTTTATGTG
TGAATTTTTTTAACTAAACTCTATCATCATTTTCTATGTTGACATCTTTTTTTTTTTTTTAAT
CTGTTTCCAACTCTGAGTCTGTGAACTATCTCTTCTGACTGGATGCTGGCCTAAAATCCTATT
AGTGCTTAAACAGACCTCAAAACACTCTGAACCTGTAGGCCAATACAGAGATGTTGTTCTTTG
ATATATCTTGGGTCCTTGATGGCTTCAACAAACAAGGTCATAATTTGTTTAAATTCAGTGTTT
TCTGTAGATACCTTCTTCTCAGTTTCATTAAGAAGAGTAACCATCTTTGGTTTTCCAAAGAAT
GGAAACTCCTACCCTAGACTTTAGACTTAGAGCGTCTGCCTTTCACGATGAGTAAGAGATCCA
GAATGTTTAGGGGATGTGCGTCAGCCCACCTGGTACAGTGGCAAAGCTGAATGCGAACTTGAG
GCCTCCTGAGGCCTGACCTTCTATGCCCCCAGCCCTCCACCCCAAGCACCTTTTGGTGTGGCT
GGAAACACATGACATTGGATGTGATTTTTCTCAAGCCCTTCACTGTGGAAGGCATGGGAGACT
GCCCAGCATTGGGCATGTGGCTGTTAACGTTTCCATTTCAAGTCCCTGATTCTTACTGGAGAA
GTTAAGGAGCCACTTAATGTTTTCACAGACCTCTGATTCTGTTGTGAATGTGGCATTCCAGTG
AGTACAACCTGCTTGCTATAAAGAAAGCAGCATATTTTGACAATTTTATTTCTTCCTTGGGTA
CTTACATTTTTATTACAAAAATGGCCGTTATAAAAAAAGACAGAAGGGCTGGGCAGTGGCTTG
CTCCCTAGAAACTGAGATTCCGAAGCAGGTGTTTCCTCTCCCCTAGACTCAGAGGTACATTTA
ATCCATCTTCTCCATTTCCTCCTTCAGGACCAGCTATGAGATTCAGTGCATTTCTAGCCCAGC
GGATGTTCATTCTCCACTAAACTTCATCTTTTACTAAGCAAAGGGGGATTATTCCATGAGGCA
GCCAGGAGCAAGGGGCCATGATATTCATGACTTTGTCTGCTGGGCATTTTTCAAAGTGTCCTT
GAATTCACTCAGCAAACAAAGCTTTCTGGAGAATCTCTCAAAACTTAGGCCCTGCTCCATTTG
GCCAAAAATGATGGCTGCTCCAAAACTCTGAACTTCTTAAAACTCCATCTGCTACATTATTTC
TGGAGTTTAAACATGACTTTTTCTGTCTTTGAGTATAGATGTGTTTGTTTAATTAACGAAGCA
CAAGTCTGTTAAGCAGAAGGCTCCAAGCTGTATTCTATACTTGGGAATCCTTGGTGCCATCTT
TATTCTACCAAGTGCCAATCACCATGGCTAAAGTGGGCGTATATTACAGCCTGTGTCCTAAGC
TTAGAAGCTTTAATGTACTTTTTTAAATGAAAAGTATTAGAGGGGGTTGAACATTGTAACTAA
AGCATAAAGTTAGACCAATTACATGCAGAGATGTTTATTTAATATTGTGTGAGCTGAGTCCTT
CTGTATAAATTATTTGCACACTTTTCTTGCATGATGAACTGATTTTTTATAGTTGTTTGTACC
AGACGGTGGCATATTTTTGTAAAAAACTTTTGACACTGAATTGCAATAAATGTTTTTCCAACA
ACA.

In some embodiments, oligonucleotides may have a region of complementarity to a mouse FBXO32 sequence, for example, as provided below (Gene ID: 67731; NCBI Ref. No: NM_026346.3

(SEQ ID NO: 506)
GACGGGGCAGCGGCCCGGGATAAATACTGCGCTCGGGCAGCCGCTCAGCATTCCCA
GAGTCAGGAGGCGACCTTCCCCAACGCCTGCGCCCCTGTGAGTGCAAGGATCCCCG
CGCCCACCCAGGATCCGCAGCCCTCCACACTAGTTGACCCACTCTTGTCCCGGTCGC
CGCCTGCGTCGTTCCCCAGCATCTTCCCAACGCGCCGCATACCCTGGGCAAGCCAGG
CCGGTTCCTGGCTGTCAATCCGTCCCGTCCGTCGGTCGCGTCCGCGCTCTGTACCAT
GCCGTTCCTTGGGCAGGACTGGCGGTCCCCGGGCCAGAGCTGGGTGAAGACGGCGG
ACGGCTGGAAGCGCTTCTTGGATGAGAAAAGCGGCAGCTTCGTGAGCGACCTCAGC
AGTTACTGCAACAAGGAGGTATACAGTAAGGAGAATCTGTTCAGCAGCCTGAACTA
CGACGTCGCAGCCAAGAAGAGAAAGAAAGACATTCAGAACAGCAAAACCAAAACT
CAGTACTTCCATCAAGAAAAGTGGATCTATGTTCACAAAGGAAGTACGAAGGAGCG
CCATGGATACTGTACTTTGGGGGAAGCTTTCAACAGACTGGACTTCTCGACTGCCAT
CCTGGATTCCAGAAGATTCAACTACGTAGTAAGGCTGTTGGAGCTGATAGCAAAGT
CACAGCTCACATCCCTGAGTGGCATCGCCCAAAAGAACTTCATGAACATTTTGGAA
AAAGTGGTACTGAAAGTTCTTGAAGACCAGCAAAACATAAGACTTATACGGGAACT
TCTCCAGACTCTCTACACATCCTTATGCACACTGGTGCAGAGAGTCGGCAAGTCTGT
GCTGGTGGGCAACATTAACATGTGGGTGTATCGGATGGAGACCATTCTACACTGGC
AGCAGCAGCTGAATAGCATCCAGATCAGCAGGCCTGCCTTCAAAGGCCTCACGATC
ACCGACCTGCCTGTGTGCTTACAACTGAACATCATGCAGAGGCTGAGTGACGGGCG
GGACCTGGTCAGCCTGGGCCAGGCAGCCCCAGACCTGCATGTGCTCAGTGAGGACC
GGCTACTGTGGAAGAGACTCTGCCAGTACCACTTCTCAGAGAGGCAGATTCGCAAG
CGTTTGATCTTGTCTGACAAAGGGCAGCTGGATTGGAAGAAGATGTATTTTAAGCTT
GTACGATGTTACCCAAGAAGAGAGCAGTATGGGGTCACCCTGCAGCTTTGCAAACA
CTGCCACATTCTCTCCTGGAAGGGCACTGACCATCCGTGCACGGCCAACAACCCAG
AGAGCTGCTCCGTCTCACTTTCCCCTCAAGACTTTATCAATTTGTTCAAGTTCTGAAT
AATCCCAGCACACGACAACACTTCAGAAGGCTTCTAATTGGATGGCTGGGAGTCGG
GACACTTCATTTGTAAATAGTGTACATTTTAAGCATTGGCTTGAAACTGCGGGGGAT
ACGTCATTGAGGAGACGTTGGCGGGGAAGAGATGCAGTTGCCGATGGAAATTTACA
AATGTGAATTCCACATGAGAACTGGTACAGAAAAGCAGAAATACTGTAAATAGACT
TTTTATTTTCCCTAACGATTTGCAAGCAAGACTATAAAGGCAAGAACTCTATGTCAG
CCATGGAAACGGAGTCCTCTTGAGTTCCCTAGGAAGAAAAAGGCAAAAAGCTCAAA
AACAAGATGGAACACTCTGTTTACAATGTGAAAATGTTGTTAAGACAAAAATAAGG
AAGAAGGAAGATGAACGCTGTCATTGAGAAACCCTTGGGCTTTGGGTTTGGATTCG
GGGTTTGTTTTCAGCAGGCCAAGAAGTATATCCACCTGAAATCTGCACGGGCTTAA
GTCCTTATCCTATGAAGATGCCACACAATGGTCTACCTCTAAAAGCATAGCGTGTTC
TCTGGCAACATACTTTATCTGGGAGGCAATGTCTGTGTTTCATGTAAGTTCTATACT
CTGTGAAGTGATCTAAGATGGGAAGGCTGTTAGGAAAAAAAAAAAAAGCCCTCTCT
TGGTTCTGACTTCCTGTCCCCCCAGTCCCTCTCAGTCCCTGCACCTCTTCTCCTTTCA
TGTTGTCACTCCTGTAGGGAACCGGGAGGCCAGCTAAAGAACAGCAGAAGTCATGA
AGAGAGAATTAGGCACCTGTGCATGCTCAGAACTTTCCATTTAACTCTTCCTTCTTC
TAGCCTGACTTCTAGGCTTGTCCGGGCTTCCTTGAGTGTCTTCATGGATAAGTGGAT
GTCTGGGACCTTAGGAGAGCCCAAGAACTGAGGCTTGAGAGACACAGTTTCTCACA
CTAGCCAGTTAAACATTCTGCCAGCTGCTGTTTCCCCTCGGCAGGCCAATGAGCTGA
GGCACTGTCTGTTCTGTAGGCCCAGCATCCAACTCAAGTCACCCTGAACCGGGACTT
TTTCACAAGCATGATTTCAAAGAAAGCCTGATAGATGTGTTCGTCTTAAACTTGTTT
CAGGACTGAGTTAGAGCAAAGATAAAGAGTCCCGGGGGTTCTTGTGAGACAGAGA
CATGAACACTGTGGAAGAAAGGAGAAGGAGATCTCAGGGCTTAAGGAGTTAATTCC
TGGGGTGGGGGAAATGTTTTTATATTTTACTTTTTACAGATTGTTGCTTTTCTACAAT
GTACATATATTTGCAATTGTATTTGCTGTTTTTTTTTTAAATCTCCAAATAAAATTGC
CACTATGCATGCTCTTGGCAAACAAGCAGAAATAGGAATTTGTTCCATGTAAATGTT
AAGGTGTGGTGTGACCTGAAGCATTGATGCGTGGGGTAGTCCTAGGAGGAGAGGAA
AGATGGCTGAGCGTGAACAAGAACAAGGCCGGGGCAGTGAACACTTCTTCCTTCAG
TCGTAGAGCACCCTCGGGTTGGTACTGTACAGGCGGATGGAGTCTGTGGGGCTCCC
TGATTTGCAGGGTCTTACATCTGTGCTTACACCAGAAGTTCTCCATGTTTTCTTTATC
AGCACCATCTCCTGGTGGCCAAATTAAGTTGAATTTAAAGGTTCGTAATGAGAAAA
GGGATGTGTAAAAGAACAAGATTTCTTTTACATGGAAAAGTTAAAAGTCTAAAGCT
GAACCTTGGAGATTACAAACATTGTAACAGAGAAGAGTTTCCTGTTTGCTTTGGGAT
TTTTGAAATCTTCTCCCCAGAAACAATTTTACATTAAGAATGTAGGGCCTTGGGGCT
GGAGAGGTGGCTCAGCAGTTAAGAGCACTGACTGCTCTTCTGAAGGTCCTGAGTTC
AATTCCTAGCAACCACATGGTGGCTCATAACCATCCATAATGAGATCTGATGGCCTC
TTCTGGTGAGTCTGAAGACAGCTACAGTGTACTTACATATAATAAATAAATAAATA
AATAAATAAATAAATAAATAAATAAATAAATAAATCTATCTTTAAAAGAAAAAGAA
TGTAGGACCTAGGTGGAAGGAAGGAAGGAAGGAAGGAAGGAAGCAAGGAAGGAA
GGAAAGCAGGCAGGCCTGCCGCCACAGTGATTCTGAGTGACAGTGTTGAGGTTAGG
GGATCGTGGCCTTTCACATGTCCTCAGCCTGCTCTGTGGTGCTTTTCAGAGATGGGA
ACTTGTAGGGACCTTGGGGTTTACAGGTCCGTCAACTTTGCCAGCTTTGAACTGCCA
GCGCAAAACTAGTTAGCCAGTCTTACAGATAGGCAGCAGTACCCAAAGTCATATAG
CATCCATACACCCCAGGCAACAATGTAAAATGGCCCTTACCCCAAGCAGCAAGGGC
ACAGCTTCTTGAAATATTTAGTAGGTGCTTGCAGGTTCTCCATGACCCTTGGTGTCA
TTGGATATGGTATGTGACACAACCGAAGAATCGTTTGACGGATGAAGAAGAGGAAA
GGAGCATGTATTGGTCGCTGTCACTGGCTGGTAGAAAACAGGAAGGATGTGGAAGG
TGGAGAGGGAGTGGGAGAGGTGTAGAAATGATGTATCTTTGATGCTGTTTAGGGTG
GGAGCAACAGAGAGAGGAGGGCAAGCAAGATACCCATATTAGGTCATGGCCAGAG
CCTCATATTCAAGTACTGGGAGTTAAAAAGAAAAGCTCTTAGGACATTAATAGTCT
ATAGAATTCTTGGCAAACCCATGCAGGACTCCCAGACTTAAAACACAAAACCTAAG
AATTCCTGACCCAGAACTGCTAGTACAAAAGCAAGAGGGGTGTGGCTATAGAAGTT
GCTGAGAGAATTGGGACTGTGTATCTCTCTACTGGACCTGACTCCCCTCCTTTGCAT
GTTGAAATGCAGAACTAGAGTTATACCCACTTTGAAGTGATTCAGAATCCAGAGAA
GGAAAAGAGTTCTGTCCAAGCAAACCAAGGGAGATGGTCACTGGTGACTTTACTTT
GGAGCAATCAGCTGAGAGTCTCTGAATGAGAACCATCGTGTGTGTGTGTGTGTGTG
TGTGTGTGTGTGTGTGTGATACAACTGAAGTTTAGGAATGCTCCTTTTATAGCCAGA
ATCAGTAAAAGAAAATCTCACTGTCACTGTCAGCTAGACCATGTCGCTACTACCATT
GCTTCAAGTGGGTATCTCAGTTGAACGGGAGGGAAAAGGTGACAGACAGAAAGGA
ACAGACATTACACAGTGGGCTCTCCAGCCCACCAGCTGAGAGGGGCTTTCTGGTTA
GTGATGATCAACTTCAGGTGGCACTGGTTTAGAGGAGACATTTTTAAGGGACAAAG
TTTCCACCACAAAAGCTTTCAACGGAAGAGCCGATCTAATTGTGACCATGAAGCCA
GCTGATGGGTGACCACACAAACCCTTCCCCGGGAGGCATTTGCTGTTGACACAGGC
TCCTTATTTCTGCCGCTTCATACGGGAGAAAAAAAAAGAGTGGGACTTTGATTTGTC
TAAGCAGTTGGTATGATTCACAGAGAGATAAATCATCACAGGAGATGGTTAGCAGT
GTATGGTTCTCGGCTTCAACGCATTGTTTATTATAAAACATGCACCTGGGTTTATATT
TTTCTGCCTGGTTATAAAGCAATAACATCTTGCTATGCAAACACTAAATTTCATGTG
TGAATTTTCAACTGTGATTTTATGTGTGCATTTTTTAAACTAAACTCTATCATTTTTC
TATGTTGACATCTGTTGTTGGTTTGGTTTTGTTTTGTTTTTAATCTGTTTCCAACTATC
TGAGTCTGTGAACCATCTCTCCTGACTGGATGCTGGCTTGAAACCTGTTAGTGCTTA
AACAGACTCAGAAACAACCCTGGACCTCTGGGGCAAACGCAGGGGTGTTACTCTTT
GATATATCATGGGCCCTCTGTGGCTTCAACAAACAAAGTCATAATTCCTCTAAATTC
TGTGTTTTCTGTAAATATTTTCTTCTTCTCAGCTCTGCTAAAATGGGGGGGGGGGG
GGGGGGGAGAGAGAGAAACTCCTATCCCAGACTTTAGACCAAAAGGGGTTGTTAG
AGAGTATGGACTCTGGCATTTCTGCATTTGAAGATGAGGAAGCCTTTCCAGAATTGG
GGGTACAGAGGCTTTCTCTGTGCTTCCTGGGAGGTCAGCAAGAGGGTTGAACAGAA
ACCTGAAGCTTCCTGATGCCAGAGCTCTTGAGCCCACCTCTCCCCCAGCACCTTGTG
GACGTGTGTAAATTCATGACACCCACTGTAATGCCTCCCAAGATCTTCACTGTGGGA
GGCATGGGAGGTGATCCCATGATGGGCTCCTGCCACAAAATCTTCCAAACCTTTTAA
GTCTGATTCCTGCTGGAGAAGTTAGAGTCCCTCTTTCATTTTCATACCCTCTTACTCT
GCTGTGAGTGCCACATTCAGCTGAGCACCACCTGCTTCCTAAGACGTAACCAGTGTG
CTGGGGAGATGGCTCAGTTGATAAAGTGCCTGCAGTGTAAGTGTGAGGACCTGAAT
TCAGGTTCCAGCATCTGTGTAAGAGCCAGGCAGGAACTTAAGTGCAAGGCGGGGCA
GGGGTGGGGATAGGTTCATTGACCAACCAGTCTTAAGAATGAAGTGGTACCCAGGC
TCATTGAGAGATCCTGTCTCAAAAAATAAAAATGGACAGAGATCTAGGGATGCACC
CAACATCTGCCGCAACCTCCCAAGTCATATGCGTGTGCACAGAAATCCCCAGATCTT
TCTTCTCCCCTGAAATGCAAAGGTACATTTAAACTATCCATCCTCTTTCTTGCCCGTC
TTTTGGCAGTGAGCCCTGCCATAACATAAGATCCAGTGAGGAGCAGTTCAGCCAGG
TTGGACAGTGATCCATTCTGTTCATCCTTGTTTGTAATAAGCAGAATTTATCCTGTAA
GGCAAGGGGTGGTGATAGCCATAATTTTTGTCGGCTGGGCTTTTTTCAAAGTGTCCT
TGAATTCAGCAAGCAAACCTTCTTTGAGAAAGCTCTCTAAACTTAGTCTCTCTCCAT
TTGGCTGGAAATAATGGATGGCCACATCCAAACTCCAAATTTCTGGATGTGTCATAT
GCCTCAGCACTACAGAATCTGACTCTCTCTTTGAGTATAGACATGCGTGTTAACTAT
GCACAGGGCTGTTTAACAGATGGCTCCAATCCCTGCTCCATACTTGGGAGCCTTTGG
TTCCATCTTTATACCAAATGCCAATCACACTGGCTACAGAGAGTGTATTATAGTCTG
TGTCTAAGCTTAGAAGCTTTAAATGTATGTTTTAAAGAAAAAATACTAGAGTGGGTT
GAACACTTTAACTAAACTTGGAAGGTTGGACAGATTATGTGCAAAGATGATTGTTT
GTTTAATAATGAATAATCTGAATCCTGTATAACTTATTTGCACACCTTTCTTGCATGA
TGAACTGACATTTTTATAGTTGTTTGTATAAGACAGTGGCATATTTTTGTAAACATTT
TTTTGACACTGAACTTCAATAAATGTTTTTCCTACAACACA.

In some embodiments, an oligonucleotide comprises a region of complementarity to a FBXO32 sequence as set forth in SEQ ID NO: 505 or SEQ ID NO: 506. In some embodiments, the oligonucleotide comprises a region of complementarity that is at least 80%, 85%, 90%, 95%, 96%, 97%, 98%, 99%, or 100% complementary to a FBXO32 sequence as set forth in SEQ ID NO: 505 or SEQ ID NO: 506. In some embodiments, the oligonucleotide comprises a sequence that has at least 10, 11, 12, 13, 14, 15, 16, 17, 18, or 19 consecutive nucleotides that are perfectly complementary to a FBXO32 sequence as set forth in SEQ ID NO: 505 or SEQ ID NO: 506. In some embodiments, an oligonucleotide may comprise a sequence that targets (e.g., is complementary to) an RNA version (i.e., wherein the T's are replaced with U's) of a FBXO32 sequence as set forth in SEQ ID NO: 505 or SEQ ID NO: 506. In some embodiments, the oligonucleotide comprises a sequence that is complementary (e.g., at least 80%, 85%, 90%, 95%, 96%, 97%, 98%, 99%, or 100% complementary) to an RNA version of a FBXO32 sequence as set forth in SEQ ID NO: 505 or SEQ ID NO: 506. In some embodiments, the oligonucleotide comprises a sequence that has at least 10, 11, 12, 13, 14, 15, 16, 17, 18, or 19 consecutive nucleotides that are perfectly complementary to an RNA version of a FBXO32 sequence as set forth in SEQ ID NO: 505 or SEQ ID NO: 506.

In some embodiments, a FBXO32-targeting oligonucleotide comprises an antisense strand that comprises at least 10, 11, 12, 13, 14, 15, 16, 17, 18, or 19 consecutive nucleotides of a sequence comprising any one of SEQ ID NOs: 555-578. In some embodiments, a FBXO32-targeting oligonucleotide comprises an antisense strand that comprises any one of SEQ ID NO: 555-578. In some embodiments, an oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 555-578.

In some embodiments, a FBXO32-targeting oligonucleotide comprises an antisense strand that targets a FBXO32 sequence comprising any one of SEQ ID NO: 507-554. In some embodiments, an oligonucleotide comprises an antisense strand comprising at least 10, 11, 12, 13, 14, 15, 16, 17, 18, or 19 nucleotides (e.g., consecutive nucleotides) that are complementary to a FBXO32 sequence comprising any one of SEQ ID NO: 507-554. In some embodiments, a FBXO32-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NO: 507-554.

In some embodiments, a FBXO32-targeting oligonucleotide comprises an antisense strand comprises a region of complementarity to a target sequence as set forth in any one of SEQ ID NOs: 507-554. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a FBXO32-targeting oligonucleotide further comprises a sense strand that hybridizes to the antisense strand to form a double stranded siRNA. In some embodiments, the FBXO32-targeting oligonucleotide comprises an antisense strand that comprises the nucleotide sequence of any one of SEQ ID NOs: 555-578. In some embodiments, the FBXO32 targeting oligonucleotide further comprises a sense strand that comprises the nucleotide sequence of any one of SEQ ID NOs: 531-554.

In some embodiments, the FBXO32-targeing oligonucleotide is a double stranded oligonucleotide (e.g., an siRNA) comprising an antisense strand that comprises the nucleotide sequence of any one of SEQ ID NOs: 555-578 and a sense strand that hybridizes to the antisense strand and comprises the nucleotide sequence of any one of SEQ ID NOs: 531-554, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the FBXO32-targeing oligonucleotide is a double stranded oligonucleotide (e.g., an siRNA) comprising an antisense strand that comprises the nucleotide sequence of any one of SEQ ID NOs: 555-578 and a sense strand that hybridizes to the antisense strand and comprises the nucleotide sequence of any one of SEQ ID NOs: 531-554, wherein the each nucleoside in the antisense strand and/or (e.g., and) each nucleoside in the sense strand is a 2′-modified nucleoside selected from 2′-O-Me and 2′-F modified nucleosides.

In some embodiments, the FBXO32-targeing oligonucleotide is a double stranded oligonucleotide (e.g., an siRNA) comprising an antisense strand that comprises the nucleotide sequence of any one of SEQ ID NOs: 555-578 and a sense strand that hybridizes to the antisense strand and comprises the nucleotide sequence of any one of SEQ ID NOs: 531-554, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the FBXO32-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the FBXO32-targeing oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the FBXO32-targeing oligonucleotide is selected from the modified version of SEQ ID NOs: 555-578 listed in Table 22. In some embodiments, the sense strand of the FBX032-targeing oligonucleotide is selected from the modified version of SEQ ID NOs: 531-554 listed in Table 22. In some embodiments, the FBX032-targeing oligonucleotide is a siRNA selected from the siRNAs listed in Table 22.

TABLE 20
FBXO32 Target Sequences

| | Corresponding | | |
|---|---|---|---|
| | nucleotides of | FBXO32 | |
| Reference | the Reference | Target Sequence | SEQ ID |
| Sequence | Sequence | (5′ to 3′) | NO: |
| NM_058229.4 | 1063-1081 | CTGGATTGGAAGAAGATGT | 507 |
| (SEQ ID NO: | | | |
| 297) | | | |
| NM_058229.4 | 1055-1073 | AAGGGCAGCTGGATTGGAA | 508 |
| (SEQ ID NO: | | | |
| 297) | | | |
| NM_058229.4 | 1064-1082 | TGGATTGGAAGAAGATGTA | 509 |
| (SEQ ID NO: | | | |
| 297) | | | |
| NM_058229.4 | 1056-1074 | AGGGCAGCTGGATTGGAAG | 510 |
| (SEQ ID NO: | | | |
| 297) | | | |
| NM_058229.4 | 1052-1070 | ACAAAGGGCAGCTGGATTG | 511 |
| (SEQ ID NO: | | | |
| 297) | | | |
| NM_058229.4 | 5127-5145 | TCATCATTTTCTATGTTGA | 512 |
| (SEQ ID NO: | | | |
| 297) | | | |
| NM_058229.4 | 2295-2313 | TGGATATCTTCATGGATAA | 513 |
| (SEQ ID NO: | | | |
| 297) | | | |
| NM_058229.4 | 5217-5235 | CTAAAATCCTATTAGTGCT | 514 |
| (SEQ ID NO: | | | |
| 297) | | | |
| NM_058229.4 | 842-860 | GGCCTGCCTTCAAAGGCCT | 515 |
| (SEQ ID NO: | | | |
| 297) | | | |
| NM_058229.4 | 1516-1534 | GCAAGACTATAAGGGCAAT | 516 |
| (SEQ ID NO: | | | |
| 297) | | | |
| NM_058229.4 | 1022-1040 | GGCAGATCCGCAAACGATT | 517 |
| (SEQ ID NO: | | | |
| 297) | | | |
| NM_058229.4 | 1448-1466 | ACTTCTCACTAGAATTGGT | 518 |
| (SEQ ID NO: | | | |
| 297) | | | |
| NM_058229.4 | 2756-2774 | TACATATATTTGCAGTTGT | 519 |
| (SEQ ID NO: | | | |
| 297) | | | |
| NM_058229.4 | 5299-5317 | CTTGGGTCCTTGATGGCTT | 520 |
| (SEQ ID NO: | | | |
| 297) | | | |
| NM_058229.4 | 4887-4905 | CTTGGAATTTCAATTGTCT | 521 |
| (SEQ ID NO: | | | |
| 297) | | | |
| NM_026346.3 | 742-760 | TGAAAGTTCTTGAAGACCA | 522 |
| (SEQ ID NO: | | | |
| 298) | | | |
| NM_026346.3 | 966-984 | CCTGTGTGCTTACAACTGA | 523 |
| (SEQ ID NO: | | | |
| 298) | | | |
| NM_026346.3 | 655-673 | TGTTGGAGCTGATAGCAAA | 524 |
| (SEQ ID NO: | | | |
| 298) | | | |
| NM_026346.3 | 621-639 | CTGGATTCCAGAAGATTCA | 525 |
| (SEQ ID NO: | | | |
| 298) | | | |
| NM_026346.3 | 628-646 | CCAGAAGATTCAACTACGT | 526 |
| (SEQ ID NO: | | | |
| 298) | | | |
| NM_026346.3 | 697-715 | GCATCGCCCAAAAGAACTT | 527 |
| (SEQ ID NO: | | | |
| 298) | | | |
| NM_026346.3 | 620-638 | CCTGGATTCCAGAAGATTC | 528 |
| (SEQ ID NO: | | | |
| 298) | | | |
| NM_026346.3 | 740-758 | ACTGAAAGTTCTTGAAGAC | 529 |
| (SEQ ID NO: | | | |
| 298) | | | |
| NM_026346.3 | 1080-1098 | AAGAGACTCTGCCAGTACC | 530 |
| (SEQ ID NO: | | | |
| 298) | | | |

*The target sequences contain Ts, but binding to RNA and/or DNA is contemplated.

In some embodiments, an oligonucleotide may comprise or consist of any sequence as provided in Table 21.

TABLE 21
Oligonucleotide sequences for targeting FBXO32

| Passenger Strand/ | | Guide Strand/ | |
|---|---|---|---|
| Sense Strand (RNA) | SEQ ID | Antisense Strand (RNA) | SEQ ID |
| (5′ to 3′) | NO: | (5′ to 3′) | NO: |
| AGCUGGAUUGGAAGAAGAUGU | 531 | ACAUCUUCUUCCAAUCCAGCUGC | 555 |
| CAAAGGGCAGCUGGAUUGGAA | 532 | UUCCAAUCCAGCUGCCCUUUGUC | 556 |
| GCUGGAUUGGAAGAAGAUGUA | 533 | UACAUCUUCUUCCAAUCCAGCUG | 557 |
| AAAGGGCAGCUGGAUUGGAAG | 534 | CUUCCAAUCCAGCUGCCCUUUGU | 558 |
| AGACAAAGGGCAGCUGGAUUG | 535 | CAAUCCAGCUGCCCUUUGUCUGA | 559 |
| UAUCAUCAUUUUCUAUGUUGA | 536 | UCAACAUAGAAAAUGAUGAUAGA | 560 |
| CUUGGAUAUCUUCAUGGAUAA | 537 | UUAUCCAUGAAGAUAUCCAAGGA | 561 |
| GCCUAAAAUCCUAUUAGUGCU | 538 | AGCACUAAUAGGAUUUUAGGCCA | 562 |
| CAGGCCUGCCUUCAAAGGCCU | 539 | AGGCCUUUGAAGGCAGGCCUGGU | 563 |
| CAGCAAGACUAUAAGGGCAAU | 540 | AUUGCCCUUAUAGUCUUGCUGGC | 564 |
| GCGGCAGAUCCGCAAACGAUU | 541 | AAUCGUUUGCGGAUCUGCCGCUC | 565 |
| GAACUUCUCACUAGAAUUGGU | 542 | ACCAAUUCUAGUGAGAAGUUCAC | 566 |
| UGUACAUAUAUUUGCAGUUGU | 543 | ACAACUGCAAAUAUAUGUACAUU | 567 |
| AUCUUGGGUCCUUGAUGGCUU | 544 | AAGCCAUCAAGGACCCAAGAUAU | 568 |
| AGCUUGGAAUUUCAAUUGUCU | 545 | AGACAAUUGAAAUUCCAAGCUCU | 569 |
| ACUGAAAGUUCUUGAAGACCA | 546 | UGGUCUUCAAGAACUUUCAGUAC | 570 |
| UGCCUGUGUGCUUACAACUGA | 547 | UCAGUUGUAAGCACACAGGCAGG | 571 |
| GCUGUUGGAGCUGAUAGCAAA | 548 | UUUGCUAUCAGCUCCAACAGCCU | 572 |
| UCCUGGAUUCCAGAAGAUUCA | 549 | UGAAUCUUCUGGAAUCCAGGAUG | 573 |
| UUCCAGAAGAUUCAACUACGU | 550 | ACGUAGUUGAAUCUUCUGGAAUC | 574 |
| UGGCAUCGCCCAAAAGAACUU | 551 | AAGUUCUUUUGGGCGAUGCCACU | 575 |
| AUCCUGGAUUCCAGAAGAUUC | 552 | GAAUCUUCUGGAAUCCAGGAUGG | 576 |
| GUACUGAAAGUUCUUGAAGAC | 553 | GUCUUCAAGAACUUUCAGUACCA | 577 |
| GGAAGAGACUCUGCCAGUACC | 554 | GGUACUGGCAGAGUCUCUUCCAC | 578 |

In some embodiments, an oligonucleotide is a modified oligonucleotide as provided in Table 22, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 22
Modified Oligonucleotides for targeting FBXO32

| | Modified Passenger | | Modified Guide | |
|---|---|---|---|---|
| | Strand/Sense Strand | SEQ ID | Strand/Antisense Strand | SEQ ID |
| siRNA # | (RNA) (5′ to 3′) | NO: | (RNA) (5′ to 3′) | NO: |
| hsFBXO32-1 | mAmGfCmUfGmGfAmUfUmG | 531 | fAfCmAfUmCfUmUfCmUfUmC | 555 |
| | fGmAfAmGfAmAfGmAfUmGf | | fCmAfAmUfCmCfAmGfCmU*f | |
| | U | | G*mC | |
| hsFBXO32-2 | mCmAfAmAfGmGfGmCfAmG | 532 | fUfUmCfCmAfAmUfCmCfAmG | 556 |
| | fCmUfGmGfAmUfUmGfGmAf | | fCmUfGmCfCmCfUmUfUmG*f | |
| | A | | U*mC | |
| hsFBXO32-3 | mGmCfUmGfGmAfUmUfGmG | 533 | fUfAmCfAmUfCmUfUmCfUmU | 557 |
| | fAmAfGmAfAmGfAmUfGmUf | | fCmCfAmAfUmCfCmAfGmC*f | |
| | A | | U*mG | |
| hsFBXO32-4 | mAmAfAmGfGmGfCmAfGmC | 534 | fCfUmUfCmCfAmAfUmCfCmA | 558 |
| | fUmGfGmAfUmUfGmGfAmAf | | fGmCfUmGfCmCfCmUfUmU*f | |
| | G | | G*mU | |
| hsFBXO32-5 | mAmGfAmCfAmAfAmGfGmG | 535 | fCfAmAfUmCfCmAfGmCfUmG | 559 |
| | fCmAfGmCfUmGfGmAfUmUf | | fCmCfCmUfUmUfGmUfCmU*f | |
| | G | | G*mA | |
| hsFBXO32-6 | mUmAfUmCfAmUfCmAfUmU | 536 | fUfCmAfAmCfAmUfAmGfAmA | 560 |
| | fUmUfCmUfAmUfGmUfUmGf | | fAmAfUmGfAmUfGmAfUmA*f | |
| | A | | G*mA | |
| hsFBXO32-7 | mCmUfUmGfGmAfUmAfUmC | 537 | fUfUmAfUmCfCmAfUmGfAmA | 561 |
| | fUmUfCmAfUmGfGmAfUmAf | | fGmAfUmAfUmCfCmAfAmG*f | |
| | A | | G*mA | |
| hsFBXO32-8 | mGmCfCmUfAmAfAmAfUmC | 538 | fAfGmCfAmCfUmAfAmUfAmG | 562 |
| | fCmUfAmUfUmAfGmUfGmCf | | fGmAfUmUfUmUfAmGfGmC*f | |
| | U | | C*mA | |
| hsFBXO32-9 | mCmAfGmGfCmCfUmGfCmCf | 539 | fAfGmGfCmCfUmUfUmGfAmA | 563 |
| | UmUfCmAfAmAfGmGfCmCf | | fGmGfCmAfGmGfCmCfUmG*f | |
| | U | | G*mU | |
| hsFBXO32- | mCmAfGmCfAmAfGmAfCmU | 540 | fAfUmUfGmCfCmCfUmUfAmU | 564 |
| 10 | fAmUfAmAfGmGfGmCfAmAf | | fAmGfUmCfUmUfGmCfUmG*f | |
| | U | | G*mC | |
| hsFBXO32- | mGmCfGmGfCmAfGmAfUmC | 541 | fAfAmUfCmGfUmUfUmGfCmG | 565 |
| 11 | fCmGfCmAfAmAfCmGfAmUf | | fGmAfUmCfUmGfCmCfGmC*f | |
| | U | | U*mC | |
| hsFBXO32- | mGmAfAmCfUmUfCmUfCmA | 542 | fAfCmCfAmAfUmUfCmUfAmG | 566 |
| 12 | fCmUfAmGfAmAfUmUfGmGf | | fUmGfAmGfAmAfGmUfUmC*f | |
| | U | | A*mC | |
| hsFBXO32- | mUmGfUmAfCmAfUmAfUmA | 543 | fAfCmAfAmCfUmGfCmAfAmA | 567 |
| 13 | fUmUfUmGfCmAfGmUfUmGf | | fUmAfUmAfUmGfUmAfCmA*f | |
| | U | | U*mU | |
| hsFBXO32- | mAmUfCmUfUmGfGmGfUmC | 544 | fAfAmGfCmCfAmUfCmAfAmG | 568 |
| 14 | fCmUfUmGfAmUfGmGfCmUf | | fGmAfCmCfCmAfAmGfAmU*f | |
| | U | | A*mU | |
| hsFBXO32- | mAmGfCmUfUmGfGmAfAmU | 545 | fAfGmAfCmAfAmUfUmGfAm | 569 |
| 15 | fUmUfCmAfAmUfUmGfUmCf | | AfAmUfUmCfCmAfAmGfCmU | |
| | U | | *fC*mU | |
| mmFBXO32- | mAmCfUmGfAmAfAmGfUmU | 546 | fUfGmGfUmCfUmUfCmAfAmG | 570 |
| 1 | fCmUfUmGfAmAfGmAfCmCf | | fAmAfCmUfUmUfCmAfGmU*f | |
| | A | | A*mC | |
| mmFBXO32- | mUmGfCmCfUmGfUmGfUmG | 547 | fUfCmAfGmUfUmGfUmAfAm | 571 |
| 2 | fCmUfUmAfCmAfAmCfUmGf | | GfCmAfCmAfCmAfGmGfCmA | |
| | A | | *fG*mG | |
| mmFBXO32- | mGmCfUmGfUmUfGmGfAmG | 548 | fUfUmUfGmCfUmAfUmCfAmG | 572 |
| 3 | fCmUfGmAfUmAfGmCfAmAf | | fCmUfCmCfAmAfCmAfGmC*f | |
| | A | | C*mU | |
| mmFBXO32- | mUmCfCmUfGmGfAmUfUmC | 549 | fUfGmAfAmUfCmUfUmCfUmG | 573 |
| 4 | fCmAfGmAfAmGfAmUfUmCf | | fGmAfAmUfCmCfAmGfGmA*f | |
| | A | | U*mG | |
| mmFBXO32- | mUmUfCmCfAmGfAmAfGmA | 550 | fAfCmGfUmAfGmUfUmGfAm | 574 |
| 5 | fUmUfCmAfAmCfUmAfCmGf | | AfUmCfUmUfCmUfGmGfAmA | |
| | U | | *fU*mC | |
| mmFBXO32- | mUmGfGmCfAmUfCmGfCmC | 551 | fAfAmGfUmUfCmUfUmUfUm | 575 |
| 6 | fCmAfAmAfAmGfAmAfCmUf | | GfGmGfCmGfAmUfGmCfCmA | |
| | U | | *fC*mU | |
| mmFBXO32- | mAmUfCmCfUmGfGmAfUmU | 552 | fGfAmAfUmCfUmUfCmUfGmG | 576 |
| 7 | fCmCfAmGfAmAfGmAfUmUf | | fAmAfUmCfCmAfGmGfAmU*f | |
| | C | | G*mG | |
| mmFBXO32- | mGmUfAmCfUmGfAmAfAmG | 553 | fGfUmCfUmUfCmAfAmGfAmA | 577 |
| 8 | fUmUfCmUfUmGfAmAfGmAf | | fCmUfUmUfCmAfGmUfAmC*f | |
| | C | | C*mA | |
| mmFBXO32- | mGmGfAmAfGmAfGmAfCmU | 554 | fGfGmUfAmCfUmGfGmCfAmG | 578 |
| 9 | fCmUfGmCfCmAfGmUfAmCf | | fAmGfUmCfUmCfUmUfCmC*f | |
| | C | | A*mC | |

g. TRIM63 Oligonucleotides

Examples of oligonucleotides useful for targeting TRJM63 are provided in Rodriguez et at., Mol Cell Endocrinol. 2015 Sep. 15; 413:36-48; Castillero et at., Metabolism. 2013 Oct; 62(10):1495-502; Clarke et al., Cell Metab. 2007 November; 6(5):376-85; Wada et al., J Biol Chem. 2011 Nov. 4; 286(44):38456-65; and Files et at., Am J Respir Crit Care Med. 2012 Apr. 15; 185(8):825-34, the contents of each of which are incorporated herein in their entireties. In some embodiments, the oligonucleotide is a CRISPR guide RNA targeting TRJM63. In some embodiments, the oligonucleotide is miR-23a, which has been shown to suppress TRJM63 expression.

In some embodiments, oligonucleotides may have a region of complementarity to a human TRJM63 sequence, for example, as provided below (Gene ID: 84676; NCBI Ref. No: NM_032588.3):

(SEQ ID NO: 579)
AGACAGAATTCGGGCACCAGGAGAAGGAAGCCAACAGGATCCGACCCGG
TGTTTTGTGACAAAGGCAAGACCCCCAGGTCTACTTAGAGCAAAGTTAG
TAGAGGAGGCAGCTAGGCGTGGCTCTCATTCCTTCCCACAGAATGGATT
ATAAGTCGAGCCTGATCCAGGATGGGAATCCCATGGAGAACTTGGAGAA
GCAGCTGATCTGCCCTATCTGCCTGGAGATGTTTACCAAGCCAGTGGTC
ATCTTGCCGTGCCAGCACAACCTGTGCCGGAAGTGTGCCAATGACATCT
TCCAGGCTGCAAATCCCTACTGGACCAGCCGGGGCAGCTCAGTGTCCAT
GTCTGGAGGCCGTTTCCGCTGCCCCACCTGCCGCCACGAGGTGATCATG
GATCGTCACGGAGTGTACGGCCTGCAGAGGAACCTGCTGGTGGAGAACA
TCATCGACATCTACAAACAGGAGTGCTCCAGTCGGCCGCTGCAGAAGGG
CAGTCACCCCATGTGCAAGGAGCACGAAGATGAGAAAATCAACATCTAC
TGTCTCACGTGTGAGGTGCCCACCTGCTCCATGTGCAAGGTGTTTGGGA
TCCACAAGGCCTGCGAGGTGGCCCCATTGCAGAGTGTCTTCCAGGGACA
AAAGACTGAACTGAATAACTGTATCTCCATGCTGGTGGCGGGGAATGAC
CGTGTGCAGACCATCATCACTCAGCTGGAGGATTCCCGTCGAGTGACCA
AGGAGAACAGTCACCAGGTAAAGGAAGAGCTGAGCCAGAAGTTTGACAC
GTTGTATGCCATCCTGGATGAGAAGAAAAGTGAGTTGCTGCAGCGGATC
ACGCAGGAGCAGGAGAAAAAGCTTAGCTTCATCGAGGCCCTCATCCAGC
AGTACCAGGAGCAGCTGGACAAGTCCACAAAGCTGGTGGAAACTGCCAT
CCAGTCCCTGGACGAGCCTGGGGGAGCCACCTTCCTCTTGACTGCCAAG
CAACTCATCAAAAGCATTGTGGAAGCTTCCAAGGGCTGCCAGCTGGGGA
AGACAGAGCAGGGCTTTGAGAACATGGACTTCTTTACTTTGGATTTAGA
GCACATAGCAGACGCCCTGAGAGCCATTGACTTTGGGACAGATGAGGAA
GAGGAAGAATTCATTGAAGAAGAAGATCAGGAAGAGGAAGAGTCCACAG
AAGGGAAGGAAGAAGGACACCAGTAAGGAGCTGGATGAATGAGAGGCCC
CCAGATGCAGAGAGACTGGAGAGGGTGGGGAGGGGCCCAGCGGCCTTGG
TGACAGGCCCAGGGTGGGAGGGGTCGGGGCCCCTGGAGGGGCAATGGGG
AGGTGATGTCTTCTCTCTGCTCAGAGAGCAGGGACTAGGGTAGGACCCT
CACCGCTGCGTCCAGCAGACACTGAACCAGAATTGGAAACGTGCTTGAA
ACAATCACACAGGACACTTTTCTACATTGGTGCAAAATGGAATATTTTG
TACATTTTTAAAATGTGATTTTTGTATATACTTGTATATGTATGCCAAT
TTGGTGCTTTTTGTAAAGGAACTTTTGTATAATAATGCCTGGTCGTTGG
GTGACCTGCGATTGTCAGAAAGAGGGGAAGGAAGCCAGGTTGATACAGC
TGCCCACTTCCTTTCCTGAGCAGGAGGATGGGGTAGCACTCACAGGGAC
GATGTGCTGTATTTCAGTGCCTATCCCAGACATACGGGGTGGTAACTGA
GTTTGTGTTATATGTTGTTTTAATAAATGCACAATGCTCTCTTCCTGTT
CTTCAAA

In some embodiments, oligonucleotides may have a region of complementarity to a mouse TRIM63 sequence, for example, as provided below (Gene ID: 433766; NCBI Ref. No: NM_001039048.2)

(SEQ ID NO: 580)
AGAAGTCGGGGGTCAGGGGACGAAGACAAAGAGGATCCGAGTGGGTTTG
GAGACAAAGACTTGGTGTGACGCAGGTGGGCGAGACAGTCGCATTTCAA
AGCAATATGGATTATAAATCTAGCCTGATTCCTGATGGAAACGCTATGG
AGAACCTGGAGAAGCAGCTGATCTGCCCCATCTGCCTGGAGATGTTTAC
CAAGCCTGTGGTCATCCTGCCCTGCCAACACAACCTCTGCCGGAAGTGT
GCCAACGACATCTTCCAGGCTGCGAATCCCTACTGGACCAACCGCGGTG
GCTCAGTGTCCATGTCTGGAGGTCGTTTCCGTTGCCCCTCGTGCCGCCA
TGAAGTGATCATGGACCGGCACGGGGTGTACGGCCTGCAGAGGAACCTG
CTGGTGGAAAACATCATTGACATCTACAAGCAGGAGTGCTCCAGTCGGC
CCCTGCAGAAAGGCAGCCACCCGATGTGCAAGGAACACGAAGACGAGAA
AATCAACATCTACTGTCTCACGTGTGAGGTGCCTACTTGCTCCTTGTGC
AAGGTGTTTGGGGCTCACCAGGCCTGTGAGGTTGCCCCTTTGCAAAGCA
TCTTCCAAGGACAGAAGACTGAGCTGAGTAACTGCATCTCCATGCTGGT
GGCGGGCAACGACCGAGTGCAGACGATCATCTCTCAGCTGGAGGACTCC
TGCAGAGTGACCAAGGAGAATAGCCACCAGGTGAAGGAGGAGCTGAGTC
AGAAGTTTGACACCCTCTACGCCATCCTGGACGAGAAGAAGAGCGAGCT
GCTGCAGCGGATCACGCAGGAGCAGGAGGAGAAGCTGGGCTTCATCGAG
GCTCTGATCCTCCAGTACAGGGAGCAGCTGGAAAAGTCCACCAAACTTG
TGGAGACCGCCATCCAGTCCCTGGATGAGCCCGGAGGGGCTACCTTCCT
CTCAAGTGCCAAGCAGCTCATCAAGAGCATTGTAGAAGCCTCCAAGGGC
TGCCAGCTGGGGAAGACAGAGCAAGGCTTTGAGAACATGGACTACTTTA
CTCTGGACTTAGAACACATAGCAGAGGCCTTGAGGGCCATTGACTTTGG
GACAGATGAGGAGGAGGAGGAGGAGGAGTTTACAGAAGAGGAGGCTGAT
GAGGAAGAGGGCGTGACCACAGAGGGTAAAGAAGAACACCAATGAAGAA
GGAGATGAGTGAGACACGCTCTGGACGCAGAGACGGGGGAGGTGGGGGC
AGGCCCATCTCGGGAGGGGGTTAGGGCTCCTTGGGGGGTACAATAGGGA
AGTGTGTCTTCTCTCTGCTCAGAGAGCAGGGACTAGCATAGGGCTCCCC
ACCACTGTGTCCAGCAGCTGCTGAAACACAATTGGAAATGTATCCAAAA
CGTCACAGGACACTTTTCTACGTTGGTGCGAAATGAAATATTTTGTATG
TTTTTAAAATGTGATTTTTGTATATACTTGTATATGTATGCCAATTTGG
TGCTTTTTGTACGAGAACTTTTGTATGATCACGCCTGGTCATTGTGTGA
CTGGCGATTGTCACAAAGTGGGAAGGAAGCCAAGACAATAGAGATGCCT
ACTTCCTTTCCTTGGTGGGAGGGCTGGGTCTCACTCGGTGGCCTAGAGA
GGGGCAATGTACTGTACAGTGCCCATCCCCAAACATGGGGATAGGACTG
AATTTGTGTTATATGTTGTTTTGCACAAGGGGCTGGGCCTTGGGGCACA
CACCCTTTGATCCCAGCACTCTGGAGGCAGAGGCAGTTGGATCTCTATG
AGTTCAAGGCCAGCCTGGTCTACATGGAGAGTTACAGGCCAGCCAGAGC
TACATAGAGAGACTCTGCCTCAAAAATAAAAATGAAAAAGAATAAAAAA
TAAACTCACAATGCTCTTTTCCTG

In some embodiments, an oligonucleotide comprises a region of complementarity to a TRIM63 sequence as set forth in SEQ ID NO: 579 or SEQ ID NO: 580. In some embodiments, the oligonucleotide comprises a region of complementarity that is at least 80%, 85%, 90%, 95%, 96%, 97%, 98%, 99%, or 100% complementary to a TRIM63 sequence as set forth in SEQ ID NO: 579 or SEQ ID NO: 580. In some embodiments, the oligonucleotide comprises a sequence that has at least 10, 11, 12, 13, 14, 15, 16, 17, 18, or 19 consecutive nucleotides that are perfectly complementary to a TRIM63 sequence as set forth in SEQ ID NO: 579 or SEQ ID NO: 580. In some embodiments, an oligonucleotide may comprise a sequence that targets (e.g., is complementary to) an RNA version (i.e., wherein the T's are replaced with U's) of a TRIM63 sequence as set forth in SEQ ID NO: 579 or SEQ ID NO: 580. In some embodiments, the oligonucleotide comprises a sequence that is complementary (e.g., at least 80%, 85%, 90%, 95%, 96%, 97%, 98%, 99%, or 100% complementary) to an RNA version of a TRIM63 sequence as set forth in SEQ ID NO: 579 or SEQ ID NO: 580. In some embodiments, the oligonucleotide comprises a sequence that has at least 10, 11, 12, 13, 14, 15, 16, 17, 18, or 19 consecutive nucleotides that are perfectly complementary to an RNA version of a TRIM63 sequence as set forth in SEQ ID NO: 579 or SEQ ID NO: 580.

In some embodiments, a TRIM63-targeting oligonucleotide comprises an antisense strand that comprises at least 10, 11, 12, 13, 14, 15, 16, 17, 18, or 19 consecutive nucleotides of a sequence comprising any one of SEQ ID NOs: 629-652. In some embodiments, a TRIM63-targeting oligonucleotide comprises an antisense strand that comprises any one of SEQ ID NO: 629-652. In some embodiments, an oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 629-652.

In some embodiments, a TRIM63-targeting oligonucleotide comprises an antisense strand that targets a TRIM63 sequence comprising any one of SEQ ID NO: 581-628. In some embodiments, an oligonucleotide comprises an antisense strand comprising at least 10, 11, 12, 13, 14, 15, 16, 17, 18, or 19 nucleotides (e.g., consecutive nucleotides) that are complementary to a TRIM63 sequence comprising any one of SEQ ID NO: 581-628. In some embodiments, a TRIM63-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NO: 581-628.

In some embodiments, a TRIM63-targeting oligonucleotide comprises an antisense strand comprises a region of complementarity to a target sequence as set forth in any one of SEQ ID NOs: 581-628. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a TRIM63-targeting oligonucleotide further comprises a sense strand that hybridizes to the antisense strand to form a double stranded siRNA. In some embodiments, the TRIM63-targeting oligonucleotide comprises an antisense strand that comprises the nucleotide sequence of any one of SEQ ID NOs: 629-652. In some embodiments, the TRIM63 targeting oligonucleotide further comprises a sense strand that comprises the nucleotide sequence of any one of SEQ ID NOs: 605-628.

In some embodiments, the TRIM63-targeing oligonucleotide is a double stranded oligonucleotide (e.g., an siRNA) comprising an antisense strand that comprises the nucleotide sequence of any one of SEQ ID NOs: 629-652 and a sense strand that hybridizes to the antisense strand and comprises the nucleotide sequence of any one of SEQ ID NOs: 605-628, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the TRIM63-targeing oligonucleotide is a double stranded oligonucleotide (e.g., an siRNA) comprising an antisense strand that comprises the nucleotide sequence of any one of SEQ ID NOs: 629-652 and a sense strand that hybridizes to the antisense strand and comprises the nucleotide sequence of any one of SEQ ID NOs: 605-628, wherein the each nucleoside in the antisense strand and/or (e.g., and) each nucleoside in the sense strand is a 2′-modified nucleoside selected from 2′-O-Me and 2′-F modified nucleosides.

In some embodiments, the TRIM63-targeing oligonucleotide is a double stranded oligonucleotide (e.g., an siRNA) comprising an antisense strand that comprises the nucleotide sequence of any one of SEQ ID NOs: 629-652 and a sense strand that hybridizes to the antisense strand and comprises the nucleotide sequence of any one of SEQ ID NOs: 605-628, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the TRIM63-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the TRIM63-targeing oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the TRIM63-targeing oligonucleotide is selected from the modified version of SEQ ID NOs: 629-652 listed in Table 25. In some embodiments, the sense strand of the TRIM63-targeing oligonucleotide is selected from the modified version of SEQ ID NOs: 605-628 listed in Table 25. In some embodiments, the TRIM63-targeing oligonucleotide is a siRNA selected from the siRNAs listed in Table 25.

TABLE 23
TRIM63 Target Sequences

| | Corresponding | | |
|---|---|---|---|
| | nucleotides of | TRIM63 | |
| | the Reference | Target Sequence | SEQ ID |
| Reference Sequence | Sequence | (5′ to 3′) | NO: |
| NM_032588.3 | 1507-1525 | TATGTATGCCAATTTGGTG | 581 |
| (SEQ ID NO: 661) | | | |
| NM_032588.3 | 531-549 | AACATCTACTGTCTCACGT | 582 |
| (SEQ ID NO: 661) | | | |
| NM_032588.3 | 1505-1523 | TATATGTATGCCAATTTGG | 583 |
| (SEQ ID NO: 661) | | | |
| NM_032588.3 | 1715-1733 | AGTTTGTGTTATATGTTGT | 584 |
| (SEQ ID NO: 661) | | | |
| NM_032588.3 | 1534-1552 | AAAGGAACTTTTGTATAAT | 585 |
| (SEQ ID NO: 661) | | | |
| NM_032588.3 | 1737-1755 | AATAAATGCACAATGCTCT | 586 |
| (SEQ ID NO: 661) | | | |
| NM_032588.3 | 138-156 | AGAATGGATTATAAGTCGA | 587 |
| (SEQ ID NO: 661) | | | |
| NM_032588.3 | 157-175 | GCCTGATCCAGGATGGGAA | 588 |
| (SEQ ID NO: 661) | | | |
| NM_032588.3 | 87-105 | GAGCAAAGTTAGTAGAGGA | 589 |
| (SEQ ID NO: 661) | | | |
| NM_032588.3 | 574-592 | GCAAGGTGTTTGGGATCCA | 590 |
| (SEQ ID NO: 661) | | | |
| NM_032588.3 | 748-766 | ACCAGGTAAAGGAAGAGCT | 591 |
| (SEQ ID NO: 661) | | | |
| NM_032588.3 | 1386-1404 | AGCAGACACTGAACCAGAA | 592 |
| (SEQ ID NO: 661) | | | |
| NM_032588.3 | 565-583 | GCTCCATGTGCAAGGTGTT | 593 |
| (SEQ ID NO: 661) | | | |
| NM_001039048.2 | 1667-1685 | AATTTGTGTTATATGTTGT | 594 |
| (SEQ ID NO: 662) | | | |
| NM_001039048.2 | 1662-1680 | GACTGAATTTGTGTTATAT | 595 |
| (SEQ ID NO: 662) | | | |
| NM_001039048.2 | 522-540 | CCTACTTGCTCCTTGTGCA | 596 |
| (SEQ ID NO: 662) | | | |
| NM_001039048.2 | 469-487 | GCAAGGAACACGAAGACGA | 597 |
| (SEQ ID NO: 662) | | | |
| NM_001039048.2 | 1009-1027 | TTGAGAACATGGACTACTT | 598 |
| (SEQ ID NO: 662) | | | |
| NM_001039048.2 | 1660-1678 | AGGACTGAATTTGTGTTAT | 599 |
| (SEQ ID NO: 662) | | | |
| NM_001039048.2 | 526-544 | CTTGCTCCTTGTGCAAGGT | 600 |
| (SEQ ID NO: 662) | | | |
| NM_001039048.2 | 237-255 | CGGAAGTGTGCCAACGACA | 601 |
| (SEQ ID NO: 662) | | | |
| NM_001039048.2 | 493-511 | TCAACATCTACTGTCTCAC | 602 |
| (SEQ ID NO: 662) | | | |
| NM_001039048.2 | 952-970 | TCAAGAGCATTGTAGAAGC | 603 |
| (SEQ ID NO: 662) | | | |
| NM_001039048.2 | 1447-1465 | ACTTGTATATGTATGCCAA | 604 |
| (SEQ ID NO: 662) | | | |

*The target sequences contain Ts, but binding to RNA and/or DNA is contemplated.

In some embodiments, an oligonucleotide may comprise or consist of any sequence as provided in Table 24.

TABLE 24
Oligonucleotide sequences for targeting TRIM63

| Passenger Strand/ | | Guide Strand/ | |
|---|---|---|---|
| Sense Strand | SEQ ID | Antisense Strand | SEQ ID |
| (RNA) (5′ to 3′) | NO: | (RNA) (5′ to 3′) | NO: |
| UAUAUGUAUGCCAAUUUGGUG | 605 | CACCAAAUUGGCAUACAUAUACA | 629 |
| UCAACAUCUACUGUCUCACGU | 606 | ACGUGAGACAGUAGAUGUUGAU | 630 |
| | | U | |
| UGUAUAUGUAUGCCAAUUUGG | 607 | CCAAAUUGGCAUACAUAUACAAG | 631 |
| UGAGUUUGUGUUAUAUGUUGU | 608 | ACAACAUAUAACACAAACUCAGU | 632 |
| GUAAAGGAACUUUUGUAUAAU | 609 | AUUAUACAAAAGUUCCUUUACAA | 633 |
| UUAAUAAAUGCACAAUGCUCU | 610 | AGAGCAUUGUGCAUUUAUUAAA | 634 |
| | | A | |
| ACAGAAUGGAUUAUAAGUCGA | 611 | UCGACUUAUAAUCCAUUCUGUGG | 635 |
| GAGCCUGAUCCAGGAUGGGAA | 612 | UUCCCAUCCUGGAUCAGGCUCGA | 636 |
| UAGAGCAAAGUUAGUAGAGGA | 613 | UCCUCUACUAACUUUGCUCUAAG | 637 |
| GUGCAAGGUGUUUGGGAUCCA | 614 | UGGAUCCCAAACACCUUGCACAU | 638 |
| UCACCAGGUAAAGGAAGAGCU | 615 | AGCUCUUCCUUUACCUGGUGACU | 639 |
| CCAGCAGACACUGAACCAGAA | 616 | UUCUGGUUCAGUGUCUGCUGGAC | 640 |
| CUGCUCCAUGUGCAAGGUGUU | 617 | AACACCUUGCACAUGGAGCAGGU | 641 |
| UGAAUUUGUGUUAUAUGUUGU | 618 | ACAACAUAUAACACAAAUUCAGU | 642 |
| AGGACUGAAUUUGUGUUAUAU | 619 | AUAUAACACAAAUUCAGUCCUAU | 643 |
| UGCCUACUUGCUCCUUGUGCA | 620 | UGCACAAGGAGCAAGUAGGCACC | 644 |
| GUGCAAGGAACACGAAGACGA | 621 | UCGUCUUCGUGUUCCUUGCACAU | 645 |
| CUUUGAGAACAUGGACUACUU | 622 | AAGUAGUCCAUGUUCUCAAAGCC | 646 |
| AUAGGACUGAAUUUGUGUUAU | 623 | AUAACACAAAUUCAGUCCUAUCC | 647 |
| UACUUGCUCCUUGUGCAAGGU | 624 | ACCUUGCACAAGGAGCAAGUAGG | 648 |
| GCCGGAAGUGUGCCAACGACA | 625 | UGUCGUUGGCACACUUCCGGCAG | 649 |
| AAUCAACAUCUACUGUCUCAC | 626 | GUGAGACAGUAGAUGUUGAUUU | 650 |
| | | U | |
| CAUCAAGAGCAUUGUAGAAGC | 627 | GCUUCUACAAUGCUCUUGAUGAG | 651 |
| AUACUUGUAUAUGUAUGCCAA | 628 | UUGGCAUACAUAUACAAGUAUAU | 652 |

In some embodiments, an oligonucleotide is a modified oligonucleotide as provided in Table 25, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 25
Modified Oligonucleotides for targeting TRIM63

| | Modified Passenger | | Modified Guide | |
|---|---|---|---|---|
| | Strand/Sense Strand | SEQ ID | Strand/Antisense Strand | SEQ ID |
| siRNA # | (RNA) | NO: | (RNA) | NO: |
| hsTRIM63-1 | mUmAfUmAfUmGfUmAfU | 605 | fCfAmCfCmAfAmAfUmUfGmGf | 629 |
| | mGfCmCfAmAfUmUfUmGf | | CmAfUmAfCmAfUmAfUmA*fC | |
| | GmUfG | | *mA | |
| hsTRIM63-2 | mUmCfAmAfCmAfUmCfUm | 606 | fAfCmGfUmGfAmGfAmCfAmGf | 630 |
| | AfCmUfGmUfCmUfCmAfC | | UmAfGmAfUmGfUmUfGmA*fU | |
| | mGfU | | *mU | |
| hsTRIM63-3 | mUmGfUmAfUmAfUmGfU | 607 | fCfCmAfAmAfUmUfGmGfCmAf | 631 |
| | mAfUmGfCmCfAmAfUmUf | | UmAfCmAfUmAfUmAfCmA*fA | |
| | UmGfG | | *mG | |
| hsTRIM63-4 | mUmGfAmGfUmUfUmGfU | 608 | fAfCmAfAmCfAmUfAmUfAmAf | 632 |
| | mGfUmUfAmUfAmUfGmUf | | CmAfCmAfAmAfCmUfCmA*fG | |
| | UmGfU | | *mU | |
| hsTRIM63-5 | mGmUfAmAfAmGfGmAfA | 609 | fAfUmUfAmUfAmCfAmAfAmAf | 633 |
| | mCfUmUfUmUfGmUfAmUf | | GmUfUmCfCmUfUmUfAmC*fA | |
| | AmAfU | | *mA | |
| hsTRIM63-6 | mUmUfAmAfUmAfAmAfU | 610 | fAfGmAfGmCfAmUfUmGfUmGf | 634 |
| | mGfCmAfCmAfAmUfGmCf | | CmAfUmUfUmAfUmUfAmA*fA | |
| | UmCfU | | *mA | |
| hsTRIM63-7 | mAmCfAmGfAmAfUmGfG | 611 | fUfCmGfAmCfUmUfAmUfAmAf | 635 |
| | mAfUmUfAmUfAmAfGmUf | | UmCfCmAfUmUfCmUfGmU*fG | |
| | CmGfA | | *mG | |
| hsTRIM63-8 | mGmAfGmCfCmUfGmAfUm | 612 | fUfUmCfCmCfAmUfCmCfUmGf | 636 |
| | CfCmAfGmGfAmUfGmGfG | | GmAfUmCfAmGfGmCfUmC*fG | |
| | mAfA | | *mA | |
| hsTRIM63-9 | mUmAfGmAfGmCfAmAfA | 613 | fUfCmCfUmCfUmAfCmUfAmAf | 637 |
| | mGfUmUfAmGfUmAfGmAf | | CmUfUmUfGmCfUmCfUmA*fA | |
| | GmGfA | | *mG | |
| hsTRIM63-10 | mGmUfGmCfAmAfGmGfU | 614 | fUfGmGfAmUfCmCfCmAfAmAf | 638 |
| | mGfUmUfUmGfGmGfAmUf | | CmAfCmCfUmUfGmCfAmC*fA | |
| | CmCfA | | *mU | |
| hsTRIM63-11 | mUmCfAmCfCmAfGmGfUm | 615 | fAfGmCfUmCfUmUfCmCfUmUf | 639 |
| | AfAmAfGmGfAmAfGmAfG | | UmAfCmCfUmGfGmUfGmA*fC | |
| | mCfU | | *mU | |
| hsTRIM63-12 | mCmCfAmGfCmAfGmAfCm | 616 | fUfUmCfUmGfGmUfUmCfAmGf | 640 |
| | AfCmUfGmAfAmCfCmAfG | | UmGfUmCfUmGfCmUfGmG*fA | |
| | mAfA | | *mC | |
| hsTRIM63-13 | mCmUfGmCfUmCfCmAfUm | 617 | fAfAmCfAmCfCmUfUmGfCmAf | 641 |
| | GfUmGfCmAfAmGfGmUfG | | CmAfUmGfGmAfGmCfAmG*fG | |
| | mUfU | | *mU | |
| mmTRIM63- | mUmGfAmAfUmUfUmGfU | 618 | fAfCmAfAmCfAmUfAmUfAmAf | 642 |
| 1 | mGfUmUfAmUfAmUfGmUf | | CmAfCmAfAmAfUmUfCmA*fG | |
| | UmGfU | | *mU | |
| mmTRIM63- | mAmGfGmAfCmUfGmAfA | 619 | fAfUmAfUmAfAmCfAmCfAmAf | 643 |
| 2 | mUfUmUfGmUfGmUfUmAf | | AmUfUmCfAmGfUmCfCmU*fA | |
| | UmAfU | | *mU | |
| mmTRIM63- | mUmGfCmCfUmAfCmUfUm | 620 | fUfGmCfAmCfAmAfGmGfAmGf | 644 |
| 3 | GfCmUfCmCfUmUfGmUfG | | CmAfAmGfUmAfGmGfCmA*fC | |
| | mCfA | | *mC | |
| mmTRIM63- | mGmUfGmCfAmAfGmGfA | 621 | fUfCmGfUmCfUmUfCmGfUmGf | 645 |
| 4 | mAfCmAfCmGfAmAfGmAf | | UmUfCmCfUmUfGmCfAmC*fA | |
| | CmGfA | | *mU | |
| mmTRIM63- | mCmUfUmUfGmAfGmAfA | 622 | fAfAmGfUmAfGmUfCmCfAmUf | 646 |
| 5 | mCfAmUfGmGfAmCfUmAf | | GmUfUmCfUmCfAmAfAmG*fC | |
| | CmUfU | | *mC | |
| mmTRIM63- | mAmUfAmGfGmAfCmUfG | 623 | fAfUmAfAmCfAmCfAmAfAmUf | 647 |
| 6 | mAfAmUfUmUfGmUfGmUf | | UmCfAmGfUmCfCmUfAmU*fC | |
| | UmAfU | | *mC | |
| mmTRIM63- | mUmAfCmUfUmGfCmUfCm | 624 | fAfCmCfUmUfGmCfAmCfAmAf | 648 |
| 7 | CfUmUfGmUfGmCfAmAfG | | GmGfAmGfCmAfAmGfUmA*fG | |
| | mGfU | | *mG | |
| mmTRIM63- | mGmCfCmGfGmAfAmGfUm | 625 | fUfGmUfCmGfUmUfGmGfCmAf | 649 |
| 8 | GfUmGfCmCfAmAfCmGfA | | CmAfCmUfUmCfCmGfGmC*fA | |
| | mCfA | | *mG | |
| mmTRIM63- | mAmAfUmCfAmAfCmAfUm | 626 | fGfUmGfAmGfAmCfAmGfUmAf | 650 |
| 9 | CfUmAfCmUfGmUfCmUfC | | GmAfUmGfUmUfGmAfUmU*fU | |
| | mAfC | | *mU | |
| mmTRIM63- | mCmAfUmCfAmAfGmAfGm | 627 | fGfCmUfUmCfUmAfCmAfAmUf | 651 |
| 10 | CfAmUfUmGfUmAfGmAfA | | GmCfUmCfUmUfGmAfUmG*fA | |
| | mGfC | | *mG | |
| mmTRIM63- | mAmUfAmCfUmUfGmUfA | 628 | fUfUmGfGmCfAmUfAmCfAmUf | 652 |
| 11 | mUfAmUfGmUfAmUfGmCf | | AmUfAmCfAmAfGmUfAmU*fA | |
| | CmAfA | | *mU | |

h. MEF2D Oligonucleotides

Examples of oligonucleotides useful for targeting MEF2D are provided in Li et al., Am J Cancer Res. 2019; 9(5): 887-905; Hu et al., Oncotarget. 2017 Nov. 3; 8(54): 92079-92089; Martis et al., BMC Cancer volume 18, Article number: 1217 (2018); Estrella et al., The Journal of Biological Chemistry, 290, 24367-24380, 2015; Ma et al., Cancer Res; 74(5) Mar. 1, 2014; and Sacilotto et al., Genes & Dev. Oct. 15, 2016 vol. 30 no. 20 2297-2309, the contents of each of which are incorporated herein in their entirety.

In some embodiments, oligonucleotides may have a region of complementarity to a human MEF2D sequence, for example, as provided below (Gene ID: 4209; NCBI Ref. No: NM_005920.4):

(SEQ ID NO: 664)
GAGTTACCTTTGGAGCCCGAAAGGAGGAAGGAAGCAAAATATCAACAACAGCCGAGGCGGCTCAGG
CGCTCGGCCCCGGTTCCCCGCTTGCCTGCCGCCCGCCTGCTGGCCCCCGCGCCCACGACGGGGGCCCA
GGCCTCACGGCGCCGCCCAGGGCCCGCGCGGACGCCGGCCTCATTTATTATTCTCCCCGCCCGGAGCT
GCGGCTTCCCGGTGTTGAAGATCCCCCGGACCAGGGGCGAGGGCTACCCGCTCTTTGCCGTGACAACA
CCGTTCCCCCAGCCGGGCTGGAGGCTGTGCAGAAGGTATCCTGCAGACCATGAACTGAGCACTGTTCC
CAGACCGTTCATGAGCACAGTGTAAGGTGTGCCGAGACCCACCACCCAGCGAGCCCCTCCCCTCCGTA
GCACTGAGGACCCCCGGAGAAGATGGGGAGGAAAAAGATTCAGATCCAGCGAATCACCGACGAGCG
GAACCGACAGGTGACTTTCACCAAGCGGAAGTTTGGCCTGATGAAGAAGGCGTATGAGCTGAGCGTG
CTATGTGACTGCGAGATCGCACTCATCATCTTCAACCACTCCAACAAGCTGTTCCAGTACGCCAGCAC
CGACATGGACAAGGTGCTGCTCAAGTACACGGAGTACAATGAGCCACACGAGAGCCGCACCAACGCC
GACATCATCGAGACCCTGAGGAAGAAGGGCTTCAACGGCTGCGACAGCCCCGAGCCCGACGGGGAG
GACTCGCTGGAACAGAGCCCCCTGCTGGAGGACAAGTACCGACGCGCCAGCGAGGAGCTCGACGGGC
TCTTCCGGCGCTATGGGTCAACTGTCCCGGCCCCCAACTTTGCCATGCCTGTCACGGTGCCCGTGTCCA
ATCAGAGCTCACTGCAGTTCAGCAATCCCAGCGGCTCCCTGGTCACCCCTTCCCTGGTGACATCATCC
CTCACGGACCCGCGGCTCCTGTCCCCCCAGCAGCCAGCACTACAGAGGAACAGTGTGTCTCCTGGCCT
GCCCCAGCGGCCAGCTAGTGCGGGGGCCATGCTGGGGGGTGACCTGAACAGTGCTAACGGAGCCTGC
CCCAGCCCTGTTGGGAATGGCTACGTCAGTGCTCGGGCTTCCCCTGGCCTCCTCCCTGTGGCCAATGG
CAACAGCCTAAACAAGGTCATCCCTGCCAAGTCTCCACCCCCACCTACCCACAGCACCCAGCTTGGAG
CCCCCAGCCGCAAGCCCGACCTGCGAGTCATCACTTCCCAGGCAGGAAAGGGGTTAATGCATCACTTG
ACTGAGGACCATTTAGATCTGAACAATGCCCAGCGCCTTGGGGTCTCCCAGTCTACTCATTCGCTCAC
CACCCCAGTGGTTTCTGTGGCAACGCCGAGTTTACTCAGCCAGGGCCTCCCCTTCTCTTCCATGCCCAC
TGCCTACAACACAGATTACCAGTTGACCAGTGCAGAGCTCTCCTCCTTACCAGCCTTTAGTTCACCTGG
GGGGCTGTCGCTAGGCAATGTCACTGCCTGGCAACAGCCACAGCAGCCCCAGCAGCCGCAGCAGCCA
CAGCCTCCACAGCAGCAGCCACCGCAGCCACAGCAGCCACAGCCACAGCAGCCTCAGCAGCCGCAAC
AGCCACCTCAGCAACAGTCCCACCTGGTCCCTGTATCTCTCAGCAACCTCATCCCGGGCAGCCCCCTG
CCCCACGTGGGTGCTGCCCTCACAGTCACCACCCACCCCCACATCAGCATCAAGTCAGAACCGGTGTC
CCCAAGCCGTGAGCGCAGCCCTGCGCCTCCCCCTCCAGCTGTGTTCCCAGCTGCCCGCCCTGAGCCTG
GCGATGGTCTCAGCAGCCCAGCCGGGGGATCCTATGAGACGGGAGACCGGGATGACGGACGGGGGG
ACTTCGGGCCCACACTGGGCCTGCTGCGCCCAGCCCCAGAGCCTGAGGCTGAGGGCTCAGCTGTGAA
GAGGATGCGGCTTGATACCTGGACATTAAAGTGACGATTCCCACTCCCCTCCTCTCAGCCTCCCTGAT
GAAGAGTTGACAATCTCACCGCCCGCCCTTCCCTGCCCCGGGCTCCTCCCGCTCGACCCCCACTTCCTT
TCTTGTGCTTCGTGTCCTGTTGACGGTTACATTTGTGTATAATTATTATATTATTATTATTATTATTATA
TTTTTTTTAATTTGGATTCTCGCTTTGGAGAGGGGGATGCTCTCATCCCCTCTTTCTGTACCCCCCACCA
TTTTCACTGGCTGGGGGGCTCTCTTTTTCGCGGGAAGGGGGGACACTTTGCACGTTGTACACATATGCT
GCAGGAAGGGGGTGGGGGGCCCAATAAGGCCTTTGGGAAAGGACAGGTGCCGAGCCCTGCATGTGG
AGCCCTCCCACCCCACCCCCAGATAGAGGGAAATAACCAAAAAACTACCAAACAACAGAAACCCACA
CTCTAGACTGAAACCCCAAAGTGGGCTTGATGGGTGGGTTTGTGTTTCAAGGGGAAAGTGAGGCAGA
GGTTCTGAAAAGGGTCTCTGTTTTTGTGTTCATGTAGCCATAGGCACATGGAGCAGAATACTTAAGCC
TGGCCCCCAAATGCCCCTGCACACACACGTGCCACACCTGCGCTGATTCTTGTGTGTGCTGCACCCCC
AAGGTGTGTGGGTGCTGGCTGAGCTTTGGGCCGGGAAGGCAGCCTGGGAATCTGAGGCTGGAGACAG
GGGTTTGAGGTGGGGGCCTCTCTGGAAGCACATTTGGAGGGAAAGACAAGAGAGCCATGAGGAGAG
GGCTGAGGAGGGCAGAAGGGCTAGGCAGGGGGCAAATTGAGCCCCTCCCTTCCCCAGTTTTTCTCTAA
GATATACAGTGCAATAGCTCCCCACCCCTCAGTTGACGCCAGCCCTGTAAAGCTGGCCACAGTGTGCA
GGGAGAATGGGGAGAGGGTCTTCAGTGAGGTGGCTGGGGCGAGAGTCGGCCTGGACTTCCCTGGGGT
GCTCCAGGCCAGAGCTCTTTCATTGGGGCGAGTGTGGTGAGGGGACGTCCTTGGTCTTGCACGCACAC
TACCTGGGGGAGTCAACACTGGGATGGTCTGTGGGGTGGGAGGGCCTACGGATGGGTCCGTAGAGGT
CCCACCTCCCTCATTCCTCCTTGGCCCCTCTCCCTAGCTTCTCCTGTTAGCTCCTTCTGCTCCTGACCCC
ACCTCCTTGCTCTTGGCGCCCCTATTGTCTCTGGCTACCTCCTTGTCCCACCACCTCCAGGCTGCATCCC
ACCTTCCCTCTTGGCTACTGTAATTGTAAATAGCGACCTTTGGAAAACGTTAGCGGTGTAACAGTCCA
GGAAACTGTTTTTTTTTGTTGTTGTTGTATTGATATGAAATGAGATTCTATTTTTGTCAAAGTATATTGT
AATAATAATGACTCAAACGGCCCGTACTGTACAGACGAGATTCTTCTGCTGTTGTTCTTGCTCCCCTCC
CCTCCTCTGAGTCCGCCCCTCCCTGCTGCCTCCTCAGTGGGGCAGTGGGCAAGGGGCCCAGGGGCAGC
CGAAGCACGGGGTCCTGAGACCTCAGGCAGGATTGGAGATCAAACCAGAGGGGGCAGGCCCCCAGC
CTGCTCTCTAGGATCACCCCCCGCCCTAAGGGGCCTGGCCTGGGGTGACGTGGCCAGGCAGACTGTCT
GCCCCACTCCTTCACACAAGCCCAGCTCCTCTGCCCAAGGGGTGCGGCGCCCCCTTGGGGTTTCCTCC
CAGTTGGAGAGTAGAGTTAAGACAAGGCCCAGTTTTGTGTTAGCCGACCGTCTTTGCCCACCTCTATG
ACCCAGCCTCTTGCAGTATTCCCATACTTGATGCAGGGAAGGAACCAGAAGCAGAGGGGCCTCTACG
CAGGTACACACGTGTACCTGAGTGTGTTCATGAGGGCATCTGGTGTTTATGTGTCTGAGTGTAGCTTTG
TATTTATGTGTGTGTGTGTGTGTATGTCTGATTGCACGGGTGTACTTTTGTATTTATGTGTGTGTGTGGT
TGCACGGGTGTGCCTCTGTGTGTCTCTGACCCTGGCTGGGTGTGTGTGCAAATCTGTGTGACTGGAGCT
CTAGGGGCATCTCTGTGTCTGAGTGTGCCTGGTGTGTGTTTACAAAGGGAGAGTTGGCTGCTCCAGCT
CCACAGCCCTGGGACCCCAACTCCTGTCTTCCCTGCTCCTTTCCCTGTGTTCACCCTCAGCTCTGACAC
ATTGAACTGCAGTTGGGGGGATTGGCAGTTAGCCCTCTGTGCTTCTCCCTGCAGCCCTACCTCTGCCAA
GGTCTCTCCCTCCAGGGACCTCTGCTTCCACCCACATATGTCCACTTAGTCACCCACACTTGACACAGT
TCCTGGAGTACCCTCTTCCCCCAACCCCAGACCTGCTTTCAGAGCAAAACTCAAGTCCCTCTTCCTCCG
TGAAGCTTCTCCCTCAGCTGAGCAGTGATCACTTACTCACTCTTAACCCCAATCCGCTGACTGGGTGG
GGACAGCACGTCCAGCCTTCCCACCTCTCCTGCAGGCTTCTAGACGGAGTTTCAAAAACTGATGAGCC
TCGATCCAGGGCTTGAAAGAAGCCAGGGTGTAATCTTGTTCATGCATGCGTCCCCAGAGCCTCGCCCA
GTGCCTGGCACATAGTAGGCACTCAATAAATGCTGAATGGGTGAATAGTTGAATGATAGGTGCTCAAT
AAATGAATGAATGGCCTTCCCTTCTCAGGCTATTCCCACCATTAGTCTGCCCACCTTTCTAGGCTGGGC
TTGGCCACCATTAAACACGGGGTGGGGGTGAGGGCCCCTGCAATTCACGGTGCAATATTCACCAGTTT
TGCCCTCTGCCTCATAAAGGCAAACCTGGCTTTTGATTACCATGTGTGGATGTTTCAGTGTCCTTTCTT
CTCTGTCCCTGGGGATGGGGTGGTCTGTGAATATGTGACATTTCTGCAGTTCAGTATCCGAAGGTTTCT
CTTGGGGGTAGGGGCTCCTGGGCGGCCAGATGAATGGGTCCCTGGGAACCCAGACCTCAGATGAGGA
CTTAATGTCTTCTTCCTCTCAAGCCAAATTCGCCTCCACCCACTCCCTCTGAAGAACTGGGCATTTGCC
AAAGTAACCACTGGAGTCATCTAATGGCCCTCCCCCTCCCCAGGTTTCCCACAGCTTTCAGGGACAGT
GGGCAAGAGGACACCCCCCCCCACCACCTCAGTGGAACACACCATTCTCCCCCCCTCAACAGCACACT
CAGTGCAGCAAGACTGACCCCTGACCCCCTCCCAGCCCTCCCTACCTTGGACAGGAAGGAAGTAATGC
ACCTTCTCTTGCTGATTATTTATTTGTTTGGAGAGACAGAAATGTAAAAGTGTATCTAGAAATATCTAT
ATCTCTATATATTTTTAACTGACTCTTTGGAATCCCCTGGGGTGGGGTGAGGGGTAAGTTTAGGCTTTC
GCGGAGGGGAGGAGACATGGAGCCTGGGAACTCCTTGTTCTCCCCTCTGCTGCCTCTCCCCACCCCTT
AAAGCAGTTGGTAGAAGGAATGGTATTTGTATGGGGGGAGGGAGGCTGGAATGGAGAATCTGGATTC
TCTCCTCTTCCCCATTCTCCAGAGGGAGGGAGGTGGTGAGGAAGAGGAAGGGAGGGGCAGGATGGGC
CATGGAGGTGCCCCACCCCCACACCTGACAATCACCCACACTCCTGGGGCTCTTCCTGGGTCCTGGGG
CAGGGCGAGTCCAAGTGTGAGGCTGTTGATTTGTTTTCAATATTTCTTTTCGTGCTGTATGGTGATGCT
TTCTTAGTATTCTACACAATAAGAAAAGACAAAGTCCTCGAGATTCTTATGAGTTTTGTTTGAAAACTC
TTTCACTATATTTGTTGTAAAGAGGTTTACTATTAAAAGAAAAAGAATACACGTTTCTGATA.

In some embodiments, oligonucleotides may have a region of complementarity to a human MEF2D sequence, for example, as provided below (Gene ID: 4209; NCBI Ref. No: NM_001271629.2):

(SEQ ID NO: 665)
ATACCGTGTGCTCCCCTTGGCAAGTCGGTTCCTGTCCCTTAAGCTGAGACTGCAGGGCCTGCCCCCCAT
TTGAGTTCTTAGGGTGCCTGGGGCCTGAGACCACCCCTGCCCTAGGCCCAGCTTTCCTGGACTGCCTG
CCCCCACAAACCAACAGCCCGCCCCCAGGTCCCCAGTCGAAGGTATCCTGCAGACCATGAACTGAGC
ACTGTTCCCAGACCGTTCATGAGCACAGTGTAAGGTGTGCCGAGACCCACCACCCAGCGAGCCCCTCC
CCTCCGTAGCACTGAGGACCCCCGGAGAAGATGGGGAGGAAAAAGATTCAGATCCAGCGAATCACCG
ACGAGCGGAACCGACAGGTGACTTTCACCAAGCGGAAGTTTGGCCTGATGAAGAAGGCGTATGAGCT
GAGCGTGCTATGTGACTGCGAGATCGCACTCATCATCTTCAACCACTCCAACAAGCTGTTCCAGTACG
CCAGCACCGACATGGACAAGGTGCTGCTCAAGTACACGGAGTACAATGAGCCACACGAGAGCCGCAC
CAACGCCGACATCATCGAGACCCTGAGGAAGAAGGGCTTCAACGGCTGCGACAGCCCCGAGCCCGAC
GGGGAGGACTCGCTGGAACAGAGCCCCCTGCTGGAGGACAAGTACCGACGCGCCAGCGAGGAGCTC
GACGGGCTCTTCCGGCGCTATGGGTCAACTGTCCCGGCCCCCAACTTTGCCATGCCTGTCACGGTGCC
CGTGTCCAATCAGAGCTCACTGCAGTTCAGCAATCCCAGCGGCTCCCTGGTCACCCCTTCCCTGGTGA
CATCATCCCTCACGGACCCGCGGCTCCTGTCCCCCCAGCAGCCAGCACTACAGAGGAACAGTGTGTCT
CCTGGCCTGCCCCAGCGGCCAGCTAGTGCGGGGGCCATGCTGGGGGGTGACCTGAACAGTGCTAACG
GAGCCTGCCCCAGCCCTGTTGGGAATGGCTACGTCAGTGCTCGGGCTTCCCCTGGCCTCCTCCCTGTG
GCCAATGGCAACAGCCTAAACAAGGTCATCCCTGCCAAGTCTCCACCCCCACCTACCCACAGCACCCA
GCTTGGAGCCCCCAGCCGCAAGCCCGACCTGCGAGTCATCACTTCCCAGGCAGGAAAGGGGTTAATG
CATCACTTGAACAATGCCCAGCGCCTTGGGGTCTCCCAGTCTACTCATTCGCTCACCACCCCAGTGGTT
TCTGTGGCAACGCCGAGTTTACTCAGCCAGGGCCTCCCCTTCTCTTCCATGCCCACTGCCTACAACACA
GATTACCAGTTGACCAGTGCAGAGCTCTCCTCCTTACCAGCCTTTAGTTCACCTGGGGGGCTGTCGCTA
GGCAATGTCACTGCCTGGCAACAGCCACAGCAGCCCCAGCAGCCGCAGCAGCCACAGCCTCCACAGC
AGCAGCCACCGCAGCCACAGCAGCCACAGCCACAGCAGCCTCAGCAGCCGCAACAGCCACCTCAGCA
ACAGTCCCACCTGGTCCCTGTATCTCTCAGCAACCTCATCCCGGGCAGCCCCCTGCCCCACGTGGGTG
CTGCCCTCACAGTCACCACCCACCCCCACATCAGCATCAAGTCAGAACCGGTGTCCCCAAGCCGTGAG
CGCAGCCCTGCGCCTCCCCCTCCAGCTGTGTTCCCAGCTGCCCGCCCTGAGCCTGGCGATGGTCTCAG
CAGCCCAGCCGGGGGATCCTATGAGACGGGAGACCGGGATGACGGACGGGGGGACTTCGGGCCCAC
ACTGGGCCTGCTGCGCCCAGCCCCAGAGCCTGAGGCTGAGGGCTCAGCTGTGAAGAGGATGCGGCTT
GATACCTGGACATTAAAGTGACGATTCCCACTCCCCTCCTCTCAGCCTCCCTGATGAAGAGTTGACAA
TCTCACCGCCCGCCCTTCCCTGCCCCGGGCTCCTCCCGCTCGACCCCCACTTCCTTTCTTGTGCTTCGTG
TCCTGTTGACGGTTACATTTGTGTATAATTATTATATTATTATTATTATTATTATATTTTTTTTAATTTGG
ATTCTCGCTTTGGAGAGGGGGATGCTCTCATCCCCTCTTTCTGTACCCCCCACCATTTTCACTGGCTGG
GGGGCTCTCTTTTTCGCGGGAAGGGGGGACACTTTGCACGTTGTACACATATGCTGCAGGAAGGGGGT
GGGGGGCCCAATAAGGCCTTTGGGAAAGGACAGGTGCCGAGCCCTGCATGTGGAGCCCTCCCACCCC
ACCCCCAGATAGAGGGAAATAACCAAAAAACTACCAAACAACAGAAACCCACACTCTAGACTGAAA
CCCCAAAGTGGGCTTGATGGGTGGGTTTGTGTTTCAAGGGGAAAGTGAGGCAGAGGTTCTGAAAAGG
GTCTCTGTTTTTGTGTTCATGTAGCCATAGGCACATGGAGCAGAATACTTAAGCCTGGCCCCCAAATG
CCCCTGCACACACACGTGCCACACCTGCGCTGATTCTTGTGTGTGCTGCACCCCCAAGGTGTGTGGGT
GCTGGCTGAGCTTTGGGCCGGGAAGGCAGCCTGGGAATCTGAGGCTGGAGACAGGGGTTTGAGGTGG
GGGCCTCTCTGGAAGCACATTTGGAGGGAAAGACAAGAGAGCCATGAGGAGAGGGCTGAGGAGGGC
AGAAGGGCTAGGCAGGGGGCAAATTGAGCCCCTCCCTTCCCCAGTTTTTCTCTAAGATATACAGTGCA
ATAGCTCCCCACCCCTCAGTTGACGCCAGCCCTGTAAAGCTGGCCACAGTGTGCAGGGAGAATGGGG
AGAGGGTCTTCAGTGAGGTGGCTGGGGCGAGAGTCGGCCTGGACTTCCCTGGGGTGCTCCAGGCCAG
AGCTCTTTCATTGGGGCGAGTGTGGTGAGGGGACGTCCTTGGTCTTGCACGCACACTACCTGGGGGAG
TCAACACTGGGATGGTCTGTGGGGTGGGAGGGCCTACGGATGGGTCCGTAGAGGTCCCACCTCCCTCA
TTCCTCCTTGGCCCCTCTCCCTAGCTTCTCCTGTTAGCTCCTTCTGCTCCTGACCCCACCTCCTTGCTCTT
GGCGCCCCTATTGTCTCTGGCTACCTCCTTGTCCCACCACCTCCAGGCTGCATCCCACCTTCCCTCTTG
GCTACTGTAATTGTAAATAGCGACCTTTGGAAAACGTTAGCGGTGTAACAGTCCAGGAAACTGTTTTT
TTTTGTTGTTGTTGTATTGATATGAAATGAGATTCTATTTTTGTCAAAGTATATTGTAATAATAATGAC
TCAAACGGCCCGTACTGTACAGACGAGATTCTTCTGCTGTTGTTCTTGCTCCCCTCCCCTCCTCTGAGT
CCGCCCCTCCCTGCTGCCTCCTCAGTGGGGCAGTGGGCAAGGGGCCCAGGGGCAGCCGAAGCACGGG
GTCCTGAGACCTCAGGCAGGATTGGAGATCAAACCAGAGGGGGCAGGCCCCCAGCCTGCTCTCTAGG
ATCACCCCCCGCCCTAAGGGGCCTGGCCTGGGGTGACGTGGCCAGGCAGACTGTCTGCCCCACTCCTT
CACACAAGCCCAGCTCCTCTGCCCAAGGGGTGCGGCGCCCCCTTGGGGTTTCCTCCCAGTTGGAGAGT
AGAGTTAAGACAAGGCCCAGTTTTGTGTTAGCCGACCGTCTTTGCCCACCTCTATGACCCAGCCTCTTG
CAGTATTCCCATACTTGATGCAGGGAAGGAACCAGAAGCAGAGGGGCCTCTACGCAGGTACACACGT
GTACCTGAGTGTGTTCATGAGGGCATCTGGTGTTTATGTGTCTGAGTGTAGCTTTGTATTTATGTGTGT
GTGTGTGTGTATGTCTGATTGCACGGGTGTACTTTTGTATTTATGTGTGTGTGTGGTTGCACGGGTGTG
CCTCTGTGTGTCTCTGACCCTGGCTGGGTGTGTGTGCAAATCTGTGTGACTGGAGCTCTAGGGGCATCT
CTGTGTCTGAGTGTGCCTGGTGTGTGTTTACAAAGGGAGAGTTGGCTGCTCCAGCTCCACAGCCCTGG
GACCCCAACTCCTGTCTTCCCTGCTCCTTTCCCTGTGTTCACCCTCAGCTCTGACACATTGAACTGCAG
TTGGGGGGATTGGCAGTTAGCCCTCTGTGCTTCTCCCTGCAGCCCTACCTCTGCCAAGGTCTCTCCCTC
CAGGGACCTCTGCTTCCACCCACATATGTCCACTTAGTCACCCACACTTGACACAGTTCCTGGAGTAC
CCTCTTCCCCCAACCCCAGACCTGCTTTCAGAGCAAAACTCAAGTCCCTCTTCCTCCGTGAAGCTTCTC
CCTCAGCTGAGCAGTGATCACTTACTCACTCTTAACCCCAATCCGCTGACTGGGTGGGGACAGCACGT
CCAGCCTTCCCACCTCTCCTGCAGGCTTCTAGACGGAGTTTCAAAAACTGATGAGCCTCGATCCAGGG
CTTGAAAGAAGCCAGGGTGTAATCTTGTTCATGCATGCGTCCCCAGAGCCTCGCCCAGTGCCTGGCAC
ATAGTAGGCACTCAATAAATGCTGAATGGGTGAATAGTTGAATGATAGGTGCTCAATAAATGAATGA
ATGGCCTTCCCTTCTCAGGCTATTCCCACCATTAGTCTGCCCACCTTTCTAGGCTGGGCTTGGCCACCA
TTAAACACGGGGTGGGGGTGAGGGCCCCTGCAATTCACGGTGCAATATTCACCAGTTTTGCCCTCTGC
CTCATAAAGGCAAACCTGGCTTTTGATTACCATGTGTGGATGTTTCAGTGTCCTTTCTTCTCTGTCCCT
GGGGATGGGGTGGTCTGTGAATATGTGACATTTCTGCAGTTCAGTATCCGAAGGTTTCTCTTGGGGGT
AGGGGCTCCTGGGCGGCCAGATGAATGGGTCCCTGGGAACCCAGACCTCAGATGAGGACTTAATGTC
TTCTTCCTCTCAAGCCAAATTCGCCTCCACCCACTCCCTCTGAAGAACTGGGCATTTGCCAAAGTAACC
ACTGGAGTCATCTAATGGCCCTCCCCCTCCCCAGGTTTCCCACAGCTTTCAGGGACAGTGGGCAAGAG
GACACCCCCCCCCACCACCTCAGTGGAACACACCATTCTCCCCCCCTCAACAGCACACTCAGTGCAGC
AAGACTGACCCCTGACCCCCTCCCAGCCCTCCCTACCTTGGACAGGAAGGAAGTAATGCACCTTCTCT
TGCTGATTATTTATTTGTTTGGAGAGACAGAAATGTAAAAGTGTATCTAGAAATATCTATATCTCTATA
TATTTTTAACTGACTCTTTGGAATCCCCTGGGGTGGGGTGAGGGGTAAGTTTAGGCTTTCGCGGAGGG
GAGGAGACATGGAGCCTGGGAACTCCTTGTTCTCCCCTCTGCTGCCTCTCCCCACCCCTTAAAGCAGTT
GGTAGAAGGAATGGTATTTGTATGGGGGGAGGGAGGCTGGAATGGAGAATCTGGATTCTCTCCTCTTC
CCCATTCTCCAGAGGGAGGGAGGTGGTGAGGAAGAGGAAGGGAGGGGCAGGATGGGCCATGGAGGT
GCCCCACCCCCACACCTGACAATCACCCACACTCCTGGGGCTCTTCCTGGGTCCTGGGGCAGGGCGAG
TCCAAGTGTGAGGCTGTTGATTTGTTTTCAATATTTCTTTTCGTGCTGTATGGTGATGCTTTCTTAGTAT
TCTACACAATAAGAAAAGACAAAGTCCTCGAGATTCTTATGAGTTTTGTTTGAAAACTCTTTCACTAT
ATTTGTTGTAAAGAGGTTTACTATTAAAAGAAAAAGAATACACGTTTCTGATA

In some embodiments, oligonucleotides may have a region of complementarity to a mouse MEF2D sequence, for example, as provided below (Gene ID: 17261; NCBI Ref. No: NM_001310587.1)

(SEQ ID NO: 666)
TGCGCGGTGCCTACGTCGGCCTCGAGTCTGGGGCTGGAGGAGTTGCCTTTGGAGCCCGAAAGGAGGA
AGGAAGCAAAATATCAACAACCGTCGAGGCGGCTTGGGCGCTCGGCTGCGTGCCTGCCGCCCGCCGC
CGCCGCCCCCGCGCGCACCAGGGGGTCCCGGCCCTGTGGCGCCGCCCAGGCCGCGTGGACGCGGCGC
CTTCGCTGTTTCCCGTCGGAGCTGCGGCTTCGCGTAACCGAGGATTCGGCGGACCGGGCCGAGGCTCC
GGGCGCCGTGACACCCCGTCCCCCCCACGGGCTGGAGGCTGTGCATAGGTGTTCTGCAGACCATGAAC
TGATCACTAGTCCCCAGACATTCATGAGCACAGTGTGAGGCTCCTGATCACCACCCAGCAGCCCCTTC
CTCTCTGGCACTAAGGACCCCCGGAGAAGATGGGGAGGAAAAAGATTCAGATCCAGCGAATCACTGA
TGAACGGAACCGCCAGGTGACCTTCACCAAGCGGAAGTTTGGACTGATGAAGAAGGCCTACGAGCTG
AGTGTGCTGTGCGACTGCGAGATCGCGCTCATCATCTTCAACCACTCCAACAAGCTGTTCCAGTATGC
CAGCACCGACATGGACAAGGTGCTGCTCAAGTACACCGAGTACAACGAGCCACACGAGAGCCGCACC
AATGCTGACATCATCGAGACCCTGAGGAAGAAGGGTTTCAACGGCTGTGACAGCCCAGAGCCGGATG
GGGAGGACTCACTGGAGCAGAGCCCCCTGCTGGAGGACAAGTACCGGCGGGCCAGTGAGGAGCTGG
ATGGGCTCTTCAGGCGCTATGGGTCATCTGTTCCGGCCCCCAACTTTGCCATGCCTGTCACAGTGCCCG
TGTCCAATCAGAGCTCCATGCAGTTCAGCAATCCAAGTAGCTCTCTGGTCACTCCTTCCCTGGTGACAT
CATCCCTTACGGACCCACGGCTCCTGTCCCCCCAGCAGCCAGCACTACAGAGAAACAGTGTTTCTCCA
GGCTTGCCCCAGCGGCCTGCTAGTGCAGGAGCCATGCTGGGTGGAGACCTCAACAGTGCTAATGGAG
CCTGCCCCAGCCCCGTTGGGAATGGCTATGTCAGTGCCCGAGCTTCCCCTGGCCTCCTCCCTGTGGCCA
ATGGCAACAGCCTAAACAAAGTCATCCCTGCCAAGTCTCCGCCCCCACCCACCCACAACACCCAGCTT
GGAGCCCCCAGCCGCAAGCCTGATCTGCGGGTCATCACTTCCCAGGGAGGCAAAGGGTTAATGCATC
ATTTGACTGAGGACCATTTAGATCTGAACAATGCCCAGCGCCTTGGGGTCTCCCAGTCTACCCACTCG
CTCACCACCCCAGTGGTTTCCGTGGCAACACCAAGTTTACTCAGCCAGGGCCTCCCCTTCTCCTCCATG
CCCACTGCCTACAACACAGATTACCAGCTGCCCAGTGCAGAGCTATCCTCCTTACCAGCCTTCAGTTC
ACCTGCAGGGCTGGCACTAGGCAATGTCACCGCCTGGCAGCAGCCCCAGCCGCCCCAGCAGCCACAA
CCGCCACAACCGCCACAGTCACAGCCACAGCCACCACAGCCACAGCCACAGCAGCCACCTCAGCAAC
AGCCCCACTTGGTCCCCGTTTCTCTCAGCAACCTCATCCCTGGCAGCCCCTTGCCTCACGTGGGTGCTG
CTCTCACAGTCACTACCCACCCCCACATCAGCATCAAGTCAGAACCAGTGTCCCCAAGTCGTGAACGC
AGCCCTGCACCTCCTCCACCAGCTGTGTTCCCAGCTGCCCGCCCTGAGCCTGGCGAAGGTCTCAGCAG
CCCAGCTGGAGGATCCTATGAGACCGGGGACCGGGATGATGGACGGGGGGACTTTGGGCCCACACTA
GGCCTGCTGCGCCCAGCCCCAGAGCCTGAGGCTGAGGGCTCAGCTGTGAAGAGGATGCGGCTGGATA
CTTGGACATTAAAGTGATGGTTCCCACTCCCTCCTTTCAGCCTCCCTGATGAAGAGTTGACAATCTCAC
CGCCCACCCCCTCCTTATCCTGGGCTCCTCCCGCTCGACCCCACTTCCTTTCTTCTGCTCCATGTCCTGT
TGATGGTTACATTTGTGTATAATTATATTATTATTATTGTTATTATTATTATTATATTTTTAAATTCGAA
TTCTCGCTTTAGAGAGAGGGATGCTCTTGCCCCTTTCTTGCCCCCACCATTTTCACTGATGGGGACTCT
CTTTATTGCGGGAAAGGGGGGACACTTTGCACGTTGTACACATATGCTGCAGGAAGGAGGGAGGCGG
TAGGCCTTGGAGAAGGACAGGTGCCGAGCCCTGCATGTGGAGCCCTCCCATCCCATTCCCAGATAGAC
GTAAAGAACCAAAAACTACCCAACAACAAAACCCAGATAGTGGACTGAAACCCAAAGCTGGCTTGAC
GATGGGCTTGTGTCTCAGGGGATAGGAGGAGGCAGTTTCTGCTTCTGGGCTGTTCACGGCTAATACCC
ATCCTGGTCCCTCCAGCTTCCTGCATACGTATCACACTCTCTGATCCTGTGTGTTACACCCCAAGATCT
GGGTGATAGAAAGGCTGCCTAGGAATCTGAGGCCAGGGAAGGGGCTTGAGGTTGGAATCTCTAGAGA
TAACATGGAGAGAGACAGCTGTAGAAAGAGGGCTGATGAGGACAGAAGGACTGGGTGTGGTGAATT
GGGACCCTACCCCCAGCTTTTCCTTTGAGATGTACAGTGCAATAGCTCCCCGTCCTTTAGCCTACCCCA
ATCCTAAAGAAAGCTGGCCATGTACTGGGAGATTTGGGAGAGGACCTTCATTGAAGTGGCTCCAGGA
TGGGCCAGAGTCGTCCTTTGGTTCCCTGGGGCACTGAGAGCAGGAGGCTTGTCAGGGGCAGGGTGAG
GAGGGCACCTCTTGGCTTTGTGCACCTGTGTCCAGAAGAGTACTTACTGGGCACATGGAGGCTCCTGT
AGATGAGGCCCGGGCCCTTCCCCAGGCCTCTGAGCTCCTGCCCCTGACCCACCTCCCAGCTCTCTGCG
TGCTCCTTGTCTCGCTACCTCCTGTCCCTCCACCTCCAGGCCACATCCCAAACCTGCCCTCTTGCTACT
GTAATTGTAAATAGAGACCTTTCAAATGTTAGTGGTGTAACCGTCCAGGAAACTGTTGTGTTTGTTGTT
GTTGTATTGATATGAAATGAGATTCTATTTTTGTCAAAGTATATTGTAATAATAATGACTCAAATGGCC
CGTACTGTACAGATGAGATTCTTCTGCTGTGTTCTTGCCTTCCTCTCCAGACTAATGCTTTGTTGCCTCC
TCAGTGGGGCTGCAGCAAGCACCAGGGGCAGTGGAAGAAGCATTGGGTCCTTGGACCTCTTGCTGGA
TCTGAGGTCAGACTGGAGGGAATAGTTTGCCCCCTCCCCAGCCTGCTGTCACCACCCCTGCCCTTCCTC
TGCTTCCCCACCTGGGGAAAGGGGATGGGAGACAGCACACACTCCCTTGGAGTTCCTCCCAGCTGGA
GGGTAGAGTTAGACAAGGCTCAGTGTTGTAGTACGATGGTCTTTGCCCACCTGTGACCCAGCCTTTTG
TAGTATTTTGACACTTGATGCAGGGAAGGAGCCAGAAGCAGAGGGGTGTGAGCATGCTCGTGGAGGC
GTATCTGGGTTCACCCTGTATTTATGTGTTTGTGTGCAGCTGTGCGGATGTACCTTTGAGTGACTCAAG
CTCCAAGGGCCTCTGTGTGAGCTGGGTGTGTTTACAAGGGATCAGCGCCTTCCAACTCTGTGGCTTCA
CCCCCTCCCCGGGCTGCTCTCAGTCCCAAAACATTTTACTAAAGTCATGGGGGACGGGTGCTAGTTGG
CCTTCCTGTTTCTCCCCACAGCCTACCTCCACCAGGGCTTCACATTTGGAGCTCTGCCTCCATATGGCT
GTCCACTAGCCCCACCCAGGCATGGTTCATCCCTGGAGTTCTGACTTGTTTATGGAGCCAACTCAAGTT
CTTCTTGCCCGGAGCTTTGCCTTTTGTAAGGCAGGGACCCTCCCTTAACCAGAGTTTACCCCAGTAGCT
GACCAAGCATGGGGTGCTACCCACATGCCTCACCTTCCCACTCCTCCTCACAAGTCCTGGAGGGAGTT
TTAAGACTTGCTTATGGCCTTGAAAGAAGCCAGGGTTGTAGCCCTGTGCTCCCCTTGAAGTAGCTGGC
ACATTGTAGGCCCTCAGTAAATGCTGAATGGGTGGATGACAGGTGCTCAATAAATGTGGATTCACGGC
CTCATCTCAGGCCCATCCTTCAAGGCTGAGCTTGGCCACCATTACAGTTGTGGGTGAAGCCCCTATAG
TTCACAGTGTGCTACTCAACAGTTTGCCATCTGCTTCCCGAAAGCAAACCTGGCTTGTGATTGCCATGT
ACGGATGCGTGCATGCACTGTGTGTGTGTGTGCGTGCGTGTGCGTGTCTGTGTCTGCACACACACGTA
TGAGTTCTTGTGTGTCTCTGTCCCTGACTGAAGGGGCAGTCTGTGAATGAGTATGTGACATTTCTGCAG
TTTAGTATCCCCAGGTTTCTCCTGGGGCTATGCAGGGGCTTCTGGCTGGCCAGGTAAATGGATCCCTG
GGAACCTAGACCTCAAATGGGGACTTATTTTCTTCCTCTCACAAGCCAAATTTGCCTACTCTCCACTCC
CTATACAGAACTGGGTATTTGCCAAAGTAGCCTCTGGCGTCATCCAGTGCCCTTCCCACCCAGGTTCC
CCTCTGCTTTCAGGGATAGTGGGACCGAGGGCCACCCCCACATGCCTCAGTTTAGCACATTTCCTCCA
TCCCCAGCAGTGCATCCACCCACCCTCCCCAACTCCAACAGGAAGGAAGAAAGAAATGCACCTTCTCT
TGCTCGTTATTTATTTGTTTGGAGAGACAGAAATGATGGAAAAGTATCTAAATCTATCTCTCTCTCTAT
TTTTACCTAACTCTCTGGAACCCCTGTGGGGGAATGTAGGCTCTCCTAAGAGGCTGGGAGCTCCTTGTT
CTACCCTGTCTGCCTCTACATAAGCAGAAGGGGTGGGATGTAATGGGGCAGGAGGGCTGAGATTCCC
AAGGAGAATCTGAATTCTCTACCCCCATTCAAGTCCCAGAAAGAGGGAAGAAGGGAGGGATGGGCCA
GCCAAGAGCTGACCCGTCCCCAAACTTGACAATCACCCACACTCCTGGCTCCTGGGTCTGGGTGAGTC
CAAGTGTGTGGCTGTTGGTTTGTTTTCATTATTTCTTTCCGTGCTGTACGGTGATGCTTTCTAGTATTCT
GCACATTAAGAAAAGTCCTCGAGATTCTTACGAGTTTGGTTTGAAAACTCTTTCACTATATTTGTTGTA
AAGAGGTTTACTATTAAAAGAAAAAAAAAAAAACATGTTTCTGATACCTCAAAAAAAAAAAAAAAA
AA.

In some embodiments, oligonucleotides may have a region of complementarity to a mouse MEF2D sequence, for example, as provided below (Gene ID: 17261; NCBI Ref. No: NM_133665.4)

(SEQ ID NO: 667)
TGCGCGGTGCCTACGTCGGCCTCGAGTCTGGGGCTGGAGGAGTTGCCTTTGGAGCCCGAAAGGAGGA
AGGAAGCAAAATATCAACAACCGTCGAGGCGGCTTGGGCGCTCGGCTGCGTGCCTGCCGCCCGCCGC
CGCCGCCCCCGCGCGCACCAGGGGGTCCCGGCCCTGTGGCGCCGCCCAGGCCGCGTGGACGCGGCGC
CTTCGCTGTTTCCCGTCGGAGCTGCGGCTTCGCGTAACCGAGGATTCGGCGGACCGGGCCGAGGCTCC
GGGCGCCGTGACACCCCGTCCCCCCCACGGGCTGGAGGCTGTGCATAGGTGTTCTGCAGACCATGAAC
TGATCACTAGTCCCCAGACATTCATGAGCACAGTGTGAGGCTCCTGATCACCACCCAGCAGCCCCTTC
CTCTCTGGCACTAAGGACCCCCGGAGAAGATGGGGAGGAAAAAGATTCAGATCCAGCGAATCACTGA
TGAACGGAACCGCCAGGTGACCTTCACCAAGCGGAAGTTTGGACTGATGAAGAAGGCCTACGAGCTG
AGTGTGCTGTGCGACTGCGAGATCGCGCTCATCATCTTCAACCACTCCAACAAGCTGTTCCAGTATGC
CAGCACCGACATGGACAAGGTGCTGCTCAAGTACACCGAGTACAACGAGCCACACGAGAGCCGCACC
AATGCTGACATCATCGAGACCCTGAGGAAGAAGGGTTTCAACGGCTGTGACAGCCCAGAGCCGGATG
GGGAGGACTCACTGGAGCAGAGCCCCCTGCTGGAGGACAAGTACCGGCGGGCCAGTGAGGAGCTGG
ATGGGCTCTTCAGGCGCTATGGGTCATCTGTTCCGGCCCCCAACTTTGCCATGCCTGTCACAGTGCCCG
TGTCCAATCAGAGCTCCATGCAGTTCAGCAATCCAAGTAGCTCTCTGGTCACTCCTTCCCTGGTGACAT
CATCCCTTACGGACCCACGGCTCCTGTCCCCCCAGCAGCCAGCACTACAGAGAAACAGTGTTTCTCCA
GGCTTGCCCCAGCGGCCTGCTAGTGCAGGAGCCATGCTGGGTGGAGACCTCAACAGTGCTAATGGAG
CCTGCCCCAGCCCCGTTGGGAATGGCTATGTCAGTGCCCGAGCTTCCCCTGGCCTCCTCCCTGTGGCCA
ATGGCAACAGCCTAAACAAAGTCATCCCTGCCAAGTCTCCGCCCCCACCCACCCACAACACCCAGCTT
GGAGCCCCCAGCCGCAAGCCTGATCTGCGGGTCATCACTTCCCAGGGAGGCAAAGGGTTAATGCATC
ATTTGAACAATGCCCAGCGCCTTGGGGTCTCCCAGTCTACCCACTCGCTCACCACCCCAGTGGTTTCCG
TGGCAACACCAAGTTTACTCAGCCAGGGCCTCCCCTTCTCCTCCATGCCCACTGCCTACAACACAGAT
TACCAGCTGCCCAGTGCAGAGCTATCCTCCTTACCAGCCTTCAGTTCACCTGCAGGGCTGGCACTAGG
CAATGTCACCGCCTGGCAGCAGCCCCAGCCGCCCCAGCAGCCACAACCGCCACAACCGCCACAGTCA
CAGCCACAGCCACCACAGCCACAGCCACAGCAGCCACCTCAGCAACAGCCCCACTTGGTCCCCGTTTC
TCTCAGCAACCTCATCCCTGGCAGCCCCTTGCCTCACGTGGGTGCTGCTCTCACAGTCACTACCCACCC
CCACATCAGCATCAAGTCAGAACCAGTGTCCCCAAGTCGTGAACGCAGCCCTGCACCTCCTCCACCAG
CTGTGTTCCCAGCTGCCCGCCCTGAGCCTGGCGAAGGTCTCAGCAGCCCAGCTGGAGGATCCTATGAG
ACCGGGGACCGGGATGATGGACGGGGGGACTTTGGGCCCACACTAGGCCTGCTGCGCCCAGCCCCAG
AGCCTGAGGCTGAGGGCTCAGCTGTGAAGAGGATGCGGCTGGATACTTGGACATTAAAGTGATGGTT
CCCACTCCCTCCTTTCAGCCTCCCTGATGAAGAGTTGACAATCTCACCGCCCACCCCCTCCTTATCCTG
GGCTCCTCCCGCTCGACCCCACTTCCTTTCTTCTGCTCCATGTCCTGTTGATGGTTACATTTGTGTATAA
TTATATTATTATTATTGTTATTATTATTATTATATTTTTAAATTCGAATTCTCGCTTTAGAGAGAGGGAT
GCTCTTGCCCCTTTCTTGCCCCCACCATTTTCACTGATGGGGACTCTCTTTATTGCGGGAAAGGGGGGA
CACTTTGCACGTTGTACACATATGCTGCAGGAAGGAGGGAGGCGGTAGGCCTTGGAGAAGGACAGGT
GCCGAGCCCTGCATGTGGAGCCCTCCCATCCCATTCCCAGATAGACGTAAAGAACCAAAAACTACCC
AACAACAAAACCCAGATAGTGGACTGAAACCCAAAGCTGGCTTGACGATGGGCTTGTGTCTCAGGGG
ATAGGAGGAGGCAGTTTCTGCTTCTGGGCTGTTCACGGCTAATACCCATCCTGGTCCCTCCAGCTTCCT
GCATACGTATCACACTCTCTGATCCTGTGTGTTACACCCCAAGATCTGGGTGATAGAAAGGCTGCCTA
GGAATCTGAGGCCAGGGAAGGGGCTTGAGGTTGGAATCTCTAGAGATAACATGGAGAGAGACAGCTG
TAGAAAGAGGGCTGATGAGGACAGAAGGACTGGGTGTGGTGAATTGGGACCCTACCCCCAGCTTTTC
CTTTGAGATGTACAGTGCAATAGCTCCCCGTCCTTTAGCCTACCCCAATCCTAAAGAAAGCTGGCCAT
GTACTGGGAGATTTGGGAGAGGACCTTCATTGAAGTGGCTCCAGGATGGGCCAGAGTCGTCCTTTGGT
TCCCTGGGGCACTGAGAGCAGGAGGCTTGTCAGGGGCAGGGTGAGGAGGGCACCTCTTGGCTTTGTG
CACCTGTGTCCAGAAGAGTACTTACTGGGCACATGGAGGCTCCTGTAGATGAGGCCCGGGCCCTTCCC
CAGGCCTCTGAGCTCCTGCCCCTGACCCACCTCCCAGCTCTCTGCGTGCTCCTTGTCTCGCTACCTCCT
GTCCCTCCACCTCCAGGCCACATCCCAAACCTGCCCTCTTGCTACTGTAATTGTAAATAGAGACCTTTC
AAATGTTAGTGGTGTAACCGTCCAGGAAACTGTTGTGTTTGTTGTTGTTGTATTGATATGAAATGAGAT
TCTATTTTTGTCAAAGTATATTGTAATAATAATGACTCAAATGGCCCGTACTGTACAGATGAGATTCTT
CTGCTGTGTTCTTGCCTTCCTCTCCAGACTAATGCTTTGTTGCCTCCTCAGTGGGGCTGCAGCAAGCAC
CAGGGGCAGTGGAAGAAGCATTGGGTCCTTGGACCTCTTGCTGGATCTGAGGTCAGACTGGAGGGAA
TAGTTTGCCCCCTCCCCAGCCTGCTGTCACCACCCCTGCCCTTCCTCTGCTTCCCCACCTGGGGAAAGG
GGATGGGAGACAGCACACACTCCCTTGGAGTTCCTCCCAGCTGGAGGGTAGAGTTAGACAAGGCTCA
GTGTTGTAGTACGATGGTCTTTGCCCACCTGTGACCCAGCCTTTTGTAGTATTTTGACACTTGATGCAG
GGAAGGAGCCAGAAGCAGAGGGGTGTGAGCATGCTCGTGGAGGCGTATCTGGGTTCACCCTGTATTT
ATGTGTTTGTGTGCAGCTGTGCGGATGTACCTTTGAGTGACTCAAGCTCCAAGGGCCTCTGTGTGAGC
TGGGTGTGTTTACAAGGGATCAGCGCCTTCCAACTCTGTGGCTTCACCCCCTCCCCGGGCTGCTCTCAG
TCCCAAAACATTTTACTAAAGTCATGGGGGACGGGTGCTAGTTGGCCTTCCTGTTTCTCCCCACAGCCT
ACCTCCACCAGGGCTTCACATTTGGAGCTCTGCCTCCATATGGCTGTCCACTAGCCCCACCCAGGCAT
GGTTCATCCCTGGAGTTCTGACTTGTTTATGGAGCCAACTCAAGTTCTTCTTGCCCGGAGCTTTGCCTT
TTGTAAGGCAGGGACCCTCCCTTAACCAGAGTTTACCCCAGTAGCTGACCAAGCATGGGGTGCTACCC
ACATGCCTCACCTTCCCACTCCTCCTCACAAGTCCTGGAGGGAGTTTTAAGACTTGCTTATGGCCTTGA
AAGAAGCCAGGGTTGTAGCCCTGTGCTCCCCTTGAAGTAGCTGGCACATTGTAGGCCCTCAGTAAATG
CTGAATGGGTGGATGACAGGTGCTCAATAAATGTGGATTCACGGCCTCATCTCAGGCCCATCCTTCAA
GGCTGAGCTTGGCCACCATTACAGTTGTGGGTGAAGCCCCTATAGTTCACAGTGTGCTACTCAACAGT
TTGCCATCTGCTTCCCGAAAGCAAACCTGGCTTGTGATTGCCATGTACGGATGCGTGCATGCACTGTG
TGTGTGTGTGCGTGCGTGTGCGTGTCTGTGTCTGCACACACACGTATGAGTTCTTGTGTGTCTCTGTCC
CTGACTGAAGGGGCAGTCTGTGAATGAGTATGTGACATTTCTGCAGTTTAGTATCCCCAGGTTTCTCCT
GGGGCTATGCAGGGGCTTCTGGCTGGCCAGGTAAATGGATCCCTGGGAACCTAGACCTCAAATGGGG
ACTTATTTTCTTCCTCTCACAAGCCAAATTTGCCTACTCTCCACTCCCTATACAGAACTGGGTATTTGC
CAAAGTAGCCTCTGGCGTCATCCAGTGCCCTTCCCACCCAGGTTCCCCTCTGCTTTCAGGGATAGTGG
GACCGAGGGCCACCCCCACATGCCTCAGTTTAGCACATTTCCTCCATCCCCAGCAGTGCATCCACCCA
CCCTCCCCAACTCCAACAGGAAGGAAGAAAGAAATGCACCTTCTCTTGCTCGTTATTTATTTGTTTGG
AGAGACAGAAATGATGGAAAAGTATCTAAATCTATCTCTCTCTCTATTTTTACCTAACTCTCTGGAACC
CCTGTGGGGGAATGTAGGCTCTCCTAAGAGGCTGGGAGCTCCTTGTTCTACCCTGTCTGCCTCTACATA
AGCAGAAGGGGTGGGATGTAATGGGGCAGGAGGGCTGAGATTCCCAAGGAGAATCTGAATTCTCTAC
CCCCATTCAAGTCCCAGAAAGAGGGAAGAAGGGAGGGATGGGCCAGCCAAGAGCTGACCCGTCCCC
AAACTTGACAATCACCCACACTCCTGGCTCCTGGGTCTGGGTGAGTCCAAGTGTGTGGCTGTTGGTTT
GTTTTCATTATTTCTTTCCGTGCTGTACGGTGATGCTTTCTAGTATTCTGCACATTAAGAAAAGTCCTCG
AGATTCTTACGAGTTTGGTTTGAAAACTCTTTCACTATATTTGTTGTAAAGAGGTTTACTATTAAAAGA
AAAAAAAAAAAACATGTTTCTGATACCTCAAAAAAAAAAAAAAAAAA

In some embodiments, the oligonucleotide may have region of complementarity to an isoform of MEF2D, for example as reported in Martin et al., Mol Cell Biol., Mar. 1994, p. 1647-1656, the contents of which are incorporated herein by reference in its entirety.

In some embodiments, an oligonucleotide comprises a region of complementarity to a MEF2D sequence as set forth in any one of SEQ ID NOs: 664-667. In some embodiments, the oligonucleotide comprises a region of complementarity that is at least 80%, 85%, 90%, 95%, 96%, 97%, 98%, 99%, or 100% complementary to a MEF2D sequence as set forth in any one of SEQ ID NOs: 664-667. In some embodiments, the oligonucleotide comprises a sequence that has at least 10, 11, 12, 13, 14, 15, 16, 17, 18, or 19 consecutive nucleotides that are perfectly complementary to a MEF2D sequence as set forth in any one of SEQ ID NOs: 664-667. In some embodiments, an oligonucleotide may comprise a sequence that targets (e.g., is complementary to) an RNA version (i.e., wherein the T's are replaced with U's) of a MEF2D sequence as set forth in any one of SEQ ID NOs: 664-667. In some embodiments, the oligonucleotide comprises a sequence that is complementary (e.g., at least 80%, 85%, 90%, 95%, 96%, 97%, 98%, 99%, or 100% complementary) to an RNA version of a MEF2D sequence as set forth in any one of SEQ ID NOs: 664-667. In some embodiments, the oligonucleotide comprises a sequence that has at least 10, 11, 12, 13, 14, 15, 16, 17, 18, or 19 consecutive nucleotides that are perfectly complementary to an RNA version of a MEF2D sequence as set forth in any one of SEQ ID NOs: 664-667.

In some embodiments, a MEF2D-targeting oligonucleotide comprises an antisense strand that comprises at least 10, 11, 12, 13, 14, 15, 16, 17, 18, or 19 consecutive nucleotides of a sequence comprising any one of SEQ ID NOs: 716-223. In some embodiments, a MEF2D-targeting oligonucleotide comprises an antisense strand that comprises any one of SEQ ID NOs: 716-223. In some embodiments, a MEF2D-targeting oligonucleotide comprises an antisense strand that comprises shares at least 70%, 75%, 80%, 85%, 90%, 95%, or 97% sequence identity with at least 12 or at least 15 consecutive nucleotides of any one SEQ ID NOs: 716-223.

In some embodiments, a MEF2D-targeting oligonucleotide comprises an antisense strand that targets a MEF2D sequence comprising any one of SEQ ID NOs: 668-715. In some embodiments, an oligonucleotide comprises an antisense strand comprising at least 10, 11, 12, 13, 14, 15, 16, 17, 18, or 19 nucleotides (e.g., consecutive nucleotides) that are complementary to a MEF2D sequence comprising any one of SEQ ID NOs: 668-715. In some embodiments, a MEF2D-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NOs: 668-715.

In some embodiments, a MEF2D-targeting oligonucleotide comprises an antisense strand comprises a region of complementarity to a target sequence as set forth in any one of SEQ ID NOs: 668-715. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a MEF2D-targeting oligonucleotide further comprises a sense strand that hybridizes to the antisense strand to form a double stranded siRNA. In some embodiments, the MEF2D-targeting oligonucleotide comprises an antisense strand that comprises the nucleotide sequence of any one of SEQ ID NOs: 716-223. In some embodiments, the MEF2D-targeting oligonucleotide further comprises a sense strand that comprises the nucleotide sequence of any one of SEQ ID NOs: 692-715.

In some embodiments, the MEF2D-targeing oligonucleotide is a double stranded oligonucleotide (e.g., an siRNA) comprising an antisense strand that comprises the nucleotide sequence of any one of SEQ ID NOs: 716-223 and a sense strand that hybridizes to the antisense strand and comprises the nucleotide sequence of any one of SEQ ID NOs: 692-715, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the MEF2D-targeing oligonucleotide is a double stranded oligonucleotide (e.g., an siRNA) comprising an antisense strand that comprises the nucleotide sequence of any one of SEQ ID NOs: 716-223 and a sense strand that hybridizes to the antisense strand and comprises the nucleotide sequence of any one of SEQ ID NOs: 692-715, wherein the each nucleoside in the antisense strand and/or (e.g., and) each nucleoside in the sense strand is a 2′-modified nucleoside selected from 2′-O-Me and 2′-F modified nucleosides.

In some embodiments, the MEF2D-targeing oligonucleotide is a double stranded oligonucleotide (e.g., an siRNA) comprising an antisense strand that comprises the nucleotide sequence of any one of SEQ ID NOs: 716-223 and a sense strand that hybridizes to the antisense strand and comprises the nucleotide sequence of any one of SEQ ID NOs: 692-715, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the MEF2D-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the MEF2D-targeing oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the MEF2D-targeing oligonucleotide is selected from the modified version of SEQ ID NOs: 716-223 listed in Table 28. In some embodiments, the sense strand of the MEF2D-targeing oligonucleotide is selected from the modified version of SEQ ID NOs: 692-715 listed in Table 28. In some embodiments, the MEF2D-targeing oligonucleotide is a siRNA selected from the siRNAs listed in Table 28.

TABLE 26
MEF2D Target Sequences

| | Corresponding | | |
|---|---|---|---|
| | nucleotides of the | MEF2D Target Sequence | SEQ ID |
| Reference sequence | Reference Sequence | (5′ to 3′) | NO: |
| NM_005920.4 | 445-463 | TTCAGATCCAGCGAATCAC | 668 |
| (SEQ ID NO: 664) | | | |
| NM_005920.4 | 616-634 | ACAAGGTGCTGCTCAAGTA | 669 |
| (SEQ ID NO: 664) | | | |
| NM_005920.4 | 1423-1441 | CCTACAACACAGATTACCA | 670 |
| (SEQ ID NO: 664) | | | |
| NM_005920.4 | 2027-2045 | ATGAAGAGTTGACAATCTC | 671 |
| (SEQ ID NO: 664) | | | |
| NM_005920.4 | 2280-2298 | ACACTTTGCACGTTGTACA | 672 |
| (SEQ ID NO: 664) | | | |
| NM_005920.4 | 2289-2307 | ACGTTGTACACATATGCTG | 673 |
| (SEQ ID NO: 664) | | | |
| NM_005920.4 | 2355-2373 | GCCGAGCCCTGCATGTGGA | 674 |
| (SEQ ID NO: 664) | | | |
| NM_005920.4 | 3385-3403 | ATATTGTAATAATAATGAC | 675 |
| (SEQ ID NO: 664) | | | |
| NM_005920.4 | 3387-3406 | ATTGTAATAATAATGACTC | 676 |
| (SEQ ID NO: 664) | | | |
| NM_005920.4 | 3349-3367 | TATTGATATGAAATGAGAT | 677 |
| (SEQ ID NO: 664) | | | |
| NM_005920.4 | 3376-3394 | TGTCAAAGTATATTGTAAT | 678 |
| (SEQ ID NO: 664) | | | |
| NM_005920.4 | 3375-3393 | TTGTCAAAGTATATTGTAA | 679 |
| (SEQ ID NO: 664) | | | |
| NM_005920.4 | 3341-3359 | TGTTGTTGTATTGATATGA | 680 |
| (SEQ ID NO: 664) | | | |
| NM_005920.4 | 1147-1165 | GCAACAGCCTAAACAAGGT | 681 |
| (SEQ ID NO: 664) | | | |
| NM_005920.4 | 1428-1446 | AACACAGATTACCAGTTGA | 682 |
| (SEQ ID NO: 664) | | | |
| NM_005920.4 | 2461-2479 | TGGGCTTGATGGGTGGGTT | 683 |
| (SEQ ID NO: 664) | | | |
| NM_005920.4 | 2840-2858 | CTAAGATATACAGTGCAAT | 684 |
| (SEQ ID NO: 664) | | | |
| NM_005920.4 | 2844-2862 | GATATACAGTGCAATAGCT | 685 |
| (SEQ ID NO: 664) | | | |
| NM_005920.4 | 2924-2942 | AGAGGGTCTTCAGTGAGGT | 686 |
| (SEQ ID NO: 664) | | | |
| NM_005920.4 | 613-625 | CCGACATGGACAAGGTGCT | 687 |
| (SEQ ID NO: 664) | | | |
| NM_133665.4 | 894-912 | ATGCAGTTCAGCAATCCAA | 688 |
| (SEQ ID NO: 667) | | | |
| NM_133665.4 | 902-920 | CAGCAATCCAAGTAGCTCT | 689 |
| (SEQ ID NO: 667) | | | |
| NM_001310587.1 | 1377-1395 | GCAACACCAAGTTTACTCA | 690 |
| (SEQ ID NO: 666) | | | |
| NM_001310587.1 | 1955-1973 | GCTGGATACTTGGACATTA | 691 |
| (SEQ ID NO: 666) | | | |

* The target sequences contain Ts, but binding to RNA and/or DNA is contemplated.

In some embodiments, an oligonucleotide may comprise or consist of any sequence as provided in Table 27.

TABLE 27
Oligonucleotide sequences for targeting MEF2D

| Passenger Strand/ | | Guide Strand/ | |
|---|---|---|---|
| Sense Strand | SEQ | Antisense Strand | SEQ |
| (RNA) | ID | (RNA) | ID |
| (5′ to 3′) | NO: | (5′ to 3′) | NO: |
| GAUUCAGAUCCAGCGAAUCAC | 692 | GUGAUUCGCUGGAUCUGAAUCUU | 716 |
| GGACAAGGUGCUGCUCAAGUA | 693 | UACUUGAGCAGCACCUUGUCCAU | 717 |
| UGCCUACAACACAGAUUACCA | 694 | UGGUAAUCUGUGUUGUAGGCAGU | 718 |
| UGAUGAAGAGUUGACAAUCUC | 695 | GAGAUUGUCAACUCUUCAUCAGG | 719 |
| GGACACUUUGCACGUUGUACA | 696 | UGUACAACGUGCAAAGUGUCCCC | 720 |
| GCACGUUGUACACAUAUGCUG | 697 | CAGCAUAUGUGUACAACGUGCAA | 721 |
| GUGCCGAGCCCUGCAUGUGGA | 698 | UCCACAUGCAGGGCUCGGCACCU | 722 |
| GUAUAUUGUAAUAAUAAUGAC | 699 | GUCAUUAUUAUUACAAUAUACUU | 723 |
| AUAUUGUAAUAAUAAUGACUC | 700 | GAGUCAUUAUUAUUACAAUAUAC | 724 |
| UGUAUUGAUAUGAAAUGAGAU | 701 | AUCUCAUUUCAUAUCAAUACAAC | 725 |
| UUUGUCAAAGUAUAUUGUAAU | 702 | AUUACAAUAUACUUUGACAAAAA | 726 |
| UUUUGUCAAAGUAUAUUGUAA | 703 | UUACAAUAUACUUUGACAAAAAU | 727 |
| GUUGUUGUUGUAUUGAUAUGA | 704 | UCAUAUCAAUACAACAACAACAA | 728 |
| UGGCAACAGCCUAAACAAGGU | 705 | ACCUUGUUUAGGCUGUUGCCAUU | 729 |
| ACAACACAGAUUACCAGUUGA | 706 | UCAACUGGUAAUCUGUGUUGUAG | 730 |
| AGUGGGCUUGAUGGGUGGGUU | 707 | AACCCACCCAUCAAGCCCACUUU | 731 |
| CUCUAAGAUAUACAGUGCAAU | 708 | AUUGCACUGUAUAUCUUAGAGAA | 732 |
| AAGAUAUACAGUGCAAUAGCU | 709 | AGCUAUUGCACUGUAUAUCUUAG | 733 |
| GGAGAGGGUCUUCAGUGAGGU | 710 | ACCUCACUGAAGACCCUCUCCCC | 734 |
| CACCGACAUGGACAAGGUGCU | 711 | AGCACCUUGUCCAUGUCGGUGCU | 735 |
| CCAUGCAGUUCAGCAAUCCAA | 712 | UUGGAUUGCUGAACUGCAUGGAG | 736 |
| UUCAGCAAUCCAAGUAGCUCU | 713 | AGAGCUACUUGGAUUGCUGAACU | 737 |
| UGGCAACACCAAGUUUACUCA | 714 | UGAGUAAACUUGGUGUUGCCACG | 738 |
| CGGCUGGAUACUUGGACAUUA | 715 | UAAUGUCCAAGUAUCCAGCCGCA | 739 |

In some embodiments, an oligonucleotide is a modified oligonucleotide as provided in Table 28, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 28
Modified Oligonucleotides for targeting MEF2D

| | Modified Passenger | | Modified Guide | |
|---|---|---|---|---|
| | Strand/Sense | SEQ | Strand/Antisense Strand | SEQ |
| | Strand (RNA) | ID | (RNA) | ID |
| siRNA # | (5′ to 3′) | NO: | (5′ to 3′) | NO: |
| hsMEF2D-1 | mGmAfUmUfCmAfGmAfU | 692 | fGfUmGfAmUfUmCfGm | 716 |
| | mCfCmAfGmCfGmAfAmUf | | CfUmGfGmAfUmCfUm | |
| | CmAfC | | GfAmAfUmC*fU*mU | |
| hsMEF2D-2 | mGmGfAmCfAmAfGmGfU | 693 | fUfAmCfUmUfGmAfGm | 717 |
| | mGfCmUfGmCfUmCfAmAf | | CfAmGfCmAfCmCfUm | |
| | GmUfA | | UfGmUfCmC*fA*mU | |
| hsMEF2D-3 | mUmGfCmCfUmAfCmAfAm | 694 | fUfGmGfUmAfAmUfCm | 718 |
| | CfAmCfAmGfAmUfUmAfC | | UfGmUfGmUfUmGfUm | |
| | mCfA | | AfGmGfCmA*fG*mU | |
| hsMEF2D-4 | mUmGfAmUfGmAfAmGfA | 695 | fGfAmGfAmUfUmGfUm | 719 |
| | mGfUmUfGmAfCmAfAmUf | | CfAmAfCmUfCmUfUm | |
| | CmUfC | | CfAmUfCmA*fG*mG | |
| hsMEF2D-5 | mGmGfAmCfAmCfUmUfUm | 696 | fUfGmUfAmCfAmAfCm | 720 |
| | GfCmAfCmGfUmUfGmUfA | | GfUmGfCmAfAmAfGm | |
| | mCfA | | UfGmUfCmC*fC*mC | |
| hsMEF2D-6 | mGmCfAmCfGmUfUmGfUm | 697 | fCfAmGfCmAfUmAfUm | 721 |
| | AfCmAfCmAfUmAfUmGfC | | GfUmGfUmAfCmAfAm | |
| | mUfG | | CfGmUfGmC*fA*mA | |
| hsMEF2D-7 | mGmUfGmCfCmGfAmGfCm | 698 | fUfCmCfAmCfAmUfGm | 722 |
| | CfCmUfGmCfAmUfGmUfG | | CfAmGfGmGfCmUfCm | |
| | mGfA | | GfGmCfAmC*fC*mU | |
| hsMEF2D-8 | mGmUfAmUfAmUfUmGfU | 699 | fGfUmCfAmUfUmAfUm | 723 |
| | mAfAmUfAmAfUmAfAmUf | | UfAmUfUmAfCmAfAm | |
| | GmAfC | | UfAmUfAmC*fU*mU | |
| hsMEF2D-9 | mAmUfAmUfUmGfUmAfA | 700 | fGfAmGfUmCfAmUfUm | 724 |
| | mUfAmAfUmAfAmUfGmAf | | AfUmUfAmUfUmAfCm | |
| | CmUfC | | AfAmUfAmU*fA*mC | |
| hsMEF2D-10 | mUmGfUmAfUmUfGmAfU | 701 | fAfUmCfUmCfAmUfUm | 725 |
| | mAfUmGfAmAfAmUfGmAf | | UfCmAfUmAfUmCfAm | |
| | GmAfU | | AfUmAfCmA*fA*mC | |
| hsMEF2D-11 | mUmUfUmGfUmCfAmAfA | 702 | fAfUmUfAmCfAmAfUm | 726 |
| | mGfUmAfUmAfUmUfGmUf | | AfUmAfCmUfUmUfGm | |
| | AmAfU | | AfCmAfAmA*fA*mA | |
| hsMEF2D-12 | mUmUfUmUfGmUfCmAfA | 703 | fUfUmAfCmAfAmUfAm | 727 |
| | mAfGmUfAmUfAmUfUmGf | | UfAmCfUmUfUmGfAm | |
| | UmAfA | | CfAmAfAmA*fA*mU | |
| hsMEF2D-13 | mGmUfUmGfUmUfGmUfU | 704 | fUfCmAfUmAfUmCfAm | 728 |
| | mGfUmAfUmUfGmAfUmAf | | AfUmAfCmAfAmCfAm | |
| | UmGfA | | AfCmAfAmC*fA*mA | |
| hsMEF2D-14 | mUmGfGmCfAmAfCmAfGm | 705 | fAfCmCfUmUfGmUfUm | 729 |
| | CfCmUfAmAfAmCfAmAfG | | UfAmGfGmCfUmGfUm | |
| | mGfU | | UfGmCfCmA*fU*mU | |
| hsMEF2D-15 | mAmCfAmAfCmAfCmAfGm | 706 | fUfCmAfAmCfUmGfGm | 730 |
| | AfUmUfAmCfCmAfGmUfU | | UfAmAfUmCfUmGfUm | |
| | mGfA | | GfUmUfGmU*fA*mG | |
| hsMEF2D-16 | mAmGfUmGfGmGfCmUfU | 707 | fAfAmCfCmCfAmCfCm | 731 |
| | mGfAmUfGmGfGmUfGmGf | | CfAmUfCmAfAmGfCm | |
| | GmUfU | | CfCmAfCmU*fU*mU | |
| hsMEF2D-17 | mCmUfCmUfAmAfGmAfUm | 708 | fAfUmUfGmCfAmCfUm | 732 |
| | AfUmAfCmAfGmUfGmCfA | | GfUmAfUmAfUmCfUm | |
| | mAfU | | UfAmGfAmG*fA*mA | |
| hsMEF2D-18 | mAmAfGmAfUmAfUmAfC | 709 | fAfGmCfUmAfUmUfGm | 733 |
| | mAfGmUfGmCfAmAfUmAf | | CfAmCfUmGfUmAfUm | |
| | GmCfU | | AfUmCfUmU*fA*mG | |
| hsMEF2D-19 | mGmGfAmGfAmGfGmGfU | 710 | fAfCmCfUmCfAmCfUm | 734 |
| | mCfUmUfCmAfGmUfGmAf | | GfAmAfGmAfCmCfCm | |
| | GmGfU | | UfCmUfCmC*fC*mC | |
| mmMEF2D-1 | mCmAfCmCfGmAfCmAfUm | 711 | fAfGmCfAmCfCmUfUm | 735 |
| | GfGmAfCmAfAmGfGmUfG | | GfUmCfCmAfUmGfUm | |
| | mCfU | | CfGmGfUmG*fC*mU | |
| mmMEF2D-2 | mCmCfAmUfGmCfAmGfUm | 712 | fUfUmGfGmAfUmUfGm | 736 |
| | UfCmAfGmCfAmAfUmCfC | | CfUmGfAmAfCmUfGm | |
| | mAfA | | CfAmUfGmG*fA*mG | |
| mmMEF2D-3 | mUmUfCmAfGmCfAmAfUm | 713 | fAfGmAfGmCfUmAfCm | 737 |
| | CfCmAfAmGfUmAfGmCfU | | UfUmGfGmAfUmUfGm | |
| | mCfU | | CfUmGfAmA*fC*mU | |
| mmMEF2D-4 | mUmGfGmCfAmAfCmAfCm | 714 | fUfGmAfGmUfAmAfAm | 738 |
| | CfAmAfGmUfUmUfAmCfU | | CfUmUfGmGfUmGfUm | |
| | mCfA | | UfGmCfCmA*fC*mG | |
| mmMEF2D-5 | mCmGfGmCfUmGfGmAfUm | 715 | fUfAmAfUmGfUmCfCm | 739 |
| | AfCmUfUmGfGmAfCmAfU | | AfAmGfUmAfUmCfCm | |
| | mUfA | | AfGmCfCmG*fC*mA | |

i. KLF15 Oligonucleotides

Examples of oligonucleotides useful for targeting KLF15 are provided in Schoger, E. et al. “CRJSPR-Mediated Activation of Endogenous Gene Expression in the Postnatal Heart.” Circ Res. 2019 Nov 15.; Jiang J. et al. “miR-190a-5p participates in the regulation of hypoxia-induced pulmonary hypertension by targeting KLF15 and can serve as a biomarker of diagnosis and prognosis in chronic obstructive pulmonary disease complicated with pulmonary hypertension.” IntJ Chron Obstruct Pulmon Dis. 2018 Nov. 20; 13:3′7′77-3′790.; Mamet, J. et al., “Intrathecal administration of AYX2 DNA-decoy produces a long-term pain treatment in rat models of chronic pain by inhibiting the KLF6, KLF9 and KLF15 transcription factors.” Mol Pain. 2017 Jan-Dec; 13:1744806917727917.; Tang, Q. et al. “Absence of miR-223-3p ameliorates hypoxia-induced injury through repressing cardiomyocyte apoptosis and oxidative stress by targeting KLF15.” Eur J Pharmacol. 2018 Dec. 15; 841:67-74.; and Honie, T. et al., “MicroRNA-133 regulates the expression of GLUT4 by targeting KLF15 and is involved in metabolic control in cardiac myocytes.” Biochem Biophys Res Commun. 2009 Nov. 13; 389(2):315-20.; the contents of each of which are incorporated herein in their entireties.

In some embodiments, oligonucleotides may have a region of complementarity to a human KLF15 sequence, for example, as provided below (Gene ID: 28999; NCBI Ref. No: NM 014079.4):

(SEQ ID NO: 740)
AGTGCTCCGCCGCAGCGACCCGCGGGCCGGCGGGCGATCGAGCCA
GCGCAGGACCCGCGGCTCGGCCCCCGGCCGCCGCCGGACCGAGAG
TCTAGCCGCCGCCCCCAGCCCAGCCCGCCCGGCCGCAGGACCGCC
GGGGCCTGGCCGCCGGTCCGGCGTGCGCCAAGTTCAGCCGCCACC
GGCACGGCCAGGCCAGCATGGTGGACCACTTACTTCCAGTGGACG
AGAACTTCTCGTCGCCAAAATGCCCAGTTGGGTATCTGGGTGATA
GGCTGGTTGGCCGGCGGGCATATCACATGCTGCCCTCACCCGTCT
CTGAAGATGACAGCGATGCCTCCAGCCCCTGCTCCTGTTCCAGTC
CCGACTCTCAAGCCCTCTGCTCCTGCTATGGTGGAGGCCTGGGCA
CCGAGAGCCAGGACAGCATCTTGGACTTCCTATTGTCCCAGGCCA
CGCTGGGCAGTGGCGGGGGCAGCGGCAGTAGCATTGGGGCCAGCA
GTGGCCCCGTGGCCTGGGGGCCCTGGCGAAGGGCAGCGGCCCCTG
TGAAGGGGGAGCATTTCTGCTTGCCCGAGTTTCCTTTGGGTGATC
CTGATGACGTCCCACGGCCCTTCCAGCCTACCCTGGAGGAGATTG
AAGAGTTTCTGGAGGAGAACATGGAGCCTGGAGTCAAGGAGGTCC
CTGAGGGCAACAGCAAGGACTTGGATGCCTGCAGCCAGCTCTCAG
CTGGGCCACACAAGAGCCACCTCCATCCTGGGTCCAGCGGGAGAG
AGCGCTGTTCCCCTCCACCAGGTGGTGCCAGTGCAGGAGGTGCCC
AGGGCCCAGGTGGGGGCCCCACGCCTGATGGCCCCATCCCAGTGT
TGCTGCAGATCCAGCCCGTGCCTGTGAAGCAGGAATCGGGCACAG
GGCCTGCCTCCCCTGGGCAAGCCCCAGAGAATGTCAAGGTTGCCC
AGCTCCTGGTCAACATCCAGGGGCAGACCTTCGCACTCGTGCCCC
AGGTGGTACCCTCCTCCAACTTGAACCTGCCCTCCAAGTTTGTGC
GCATTGCCCCTGTGCCCATTGCCGCCAAGCCTGTTGGATCGGGAC
CCCTGGGGCCTGGCCCTGCCGGTCTCCTCATGGGCCAGAAGTTCC
CCAAGAACCCAGCCGCAGAACTCATCAAAATGCACAAATGTACTT
TCCCTGGCTGCAGCAAGATGTACACCAAAAGCAGCCACCTCAAGG
CCCACCTGCGCCGGCACACGGGTGAGAAGCCCTTCGCCTGCACCT
GGCCAGGCTGCGGCTGGAGGTTCTCGCGCTCTGACGAGCTGTCGC
GGCACAGGCGCTCGCACTCAGGTGTGAAGCCGTACCAGTGTCCTG
TGTGCGAGAAGAAGTTCGCGCGGAGCGACCACCTCTCCAAGCACA
TCAAGGTGCACCGCTTCCCGCGGAGCAGCCGCTCCGTGCGCTCCG
TGAACTGAAAGCGCCCTGAACCCCAGCCTGTCCGTCACCCCGGAT
CCCCACCCCATCCCCATTTTTTTAAGCAATAATTTATTTGCCTCC
TCCAGAGGGACATGGCAATGTTACCAGCCCACCTTCTGAAGCCTG
GGAGGTGTGAACCCAGGGCCCGCCAACCGCTGCCTTTCTCGGGAG
TACTTAGAGCCTCGAACCCGCGTCCCTGGGGGCTGGGCCCCAGGC
GCACGGGGCTGGAGGCAGGCCTTCGTGCCTTCGTGCCTTCGTGCC
TTCCCGCGGTGGCCAGGCCTCTGCTGCAGCCGCTGGTTGCAGGCA
GAGTTTTGGGGACCTGGCCCTTCTCCCACTGGGCTCCCCCATCCT
GGGCCAAGGCCAGAACTTTAGTGCTAGGGGAAGATGAAATGTGCA
GTTTTGAAATGTTGGGTTTCCAGAGAGAGTCATGCTGGAGGAGAA
GGAAGTAGGCCAGAAGTCCAGGGCTGCACTGTGGTGTGAGGGTGG
CTTTGTCTAAGATGCCTGCTCAGCATGATCACCAGAGGGTGTGGG
CAGGTCCCTGGAGCCGGGGGGGGGGGGGGGGGGGGGGGGGGGCAG
GACCGGGCCGCTGGGCCCTCATGTGGGAGAGAGGTGAAAAGCGTC
CCCCACTAGGGGGCTGGCAGTGCATGTGCTTGAGTTAAATGTGCA
GGGCAGACAGAGCCAGAAGGGCCTGTACCCAGGGGCTCGTCCCCT
CCTCCGGTTTCCCAGACAAATCCAGACACCAGCCTTTAGGGTGGC
CTTGGGAGGAGAGGGCCAGGCTGTCCTGGGTGTGAGAGAACTAGA
TAGAGCCTCCCAACCCTGATTTAGAAATGCATTCCTTATTTTGTC
TAGAAATTAATAAATGAACTAGCTTGTTTTGACAGGTTTATTTCA
CATCCTATGAATGTATGTAAATAAACTGTACATAGGTCCATCCAC
ATAAAATATCTTTTAATAACATATCAACATTTGTGTAAATTTGAA
ATTTAAAAAAATCTATGAAGCTGGTGTACATATGTTACAATTACG
TATATTTTCTTTGGTCCTTCATAAAAATATATTTACTTTGCCAAT
AAAAAGAAAAAGAACTCACA

In some embodiments, oligonucleotides may have a region of complementarity to a mouse KLF15 sequence, for example, as provided below (Gene ID: 66277; NCBI Ref. No: NM_023184.4)

(SEQ ID NO: 741)
GTGAGCGGCCGGAGAGCGGGGAGAGCGGCGAGCGGCGAGCGCCCA
GCTCGGCCGCAGGGAGTGCGGGCTGCGGCCAGGAGCGCTGGACGC
GCGGCCGCATACGGAGCCGGGCCGCACGCGCGAGGCCGCGCCAGG
CGACGCGGAGTCCAGTCACCACAGGCTCGGCCAGGCCAGCATGGT
GGACCACCTGCTTCCAGTGGACGAGACCTTCTCGTCACCGAAATG
CTCAGTGGGTTACCTAGGGGACAGGCTGGCCAGCCGGCAGCCATA
CCACATGTTGCCCTCGCCCATCTCGGAGGATGACAGCGATGTCTC
CAGCCCCTGCTCTTGTGCCAGCCCTGACTCGCAAGCCTTCTGTTC
CTGCTACAGTGCGGGTCCAGGCCCTGAGGCCCAGGGCAGCATCTT
GGATTTCCTCCTGTCCCGGGCCACACTGGGCAGTGGTGGTGGCAG
TGGAGGTATTGGAGATAGCAGTGGCCCTGTGACCTGGGGATCATG
GAGGAGAGCCTCTGTGCCTGTGAAGGAGGAACATTTCTGCTTCCC
TGAATTTCTGTCAGGGGACACTGATGACGTCTCCAGGCCCTTCCA
GCCTACCCTGGAGGAGATTGAAGAATTCCTGGAAGAGAACATGGA
GGCTGAGGTCAAGGAGGCCCCAGAGAACGGTAGCAGGGACCTGGA
GACCTGTAGCCAGCTCTCAGCTGGGTCACACCGGAGCCACCTTCA
TCCAGAGTCTGCTGGGAGAGAGCGCTGTACCCCACCACCAGGTGG
CACGAGTGGGGGTGGTGCCCAAAGTGCAGGTGAGGGGCCAGCACA
TGATGGCCCCGTGCCGGTGCTACTGCAGATCCAGCCTGTTGCTGT
GAAGCAGGAGGCAGGTACAGGGCCAGCCTCCCCAGGGCAGGCCCC
AGAGAGCGTCAAGGTCGCCCAGCTTCTAGTCAACATCCAGGGGCA
GACCTTTGCACTCCTGCCTCAAGTGGTACCATCCTCCAACTTGAA
CCTGCCCTCAAAGTTTGTGCGAATTGCGCCTGTGCCCATTGCCGC
CAAACCTATTGGCTCAGGATCCCTAGGGCCCGGCCCTGCTGGCCT
CCTTGTGGGCCAGAAGTTTCCCAAGAACCCAGCAGCAGAACTTCT
CAAAATGCACAAATGCACTTTCCCAGGCTGCAGCAAGATGTACAC
CAAGAGCAGCCACCTCAAGGCCCACCTGCGTCGGCACACAGGCGA
GAAGCCCTTTGCCTGCACCTGGCCAGGCTGCGGCTGGAGGTTTTC
CCGCTCAGATGAGTTGTCAAGGCACCGGCGATCTCACTCGGGTGT
GAAGCCGTACCAGTGTCCCGTGTGCGAGAAGAAATTCGCGCGGAG
TGACCACCTCTCCAAACACATCAAAGTGCATCGCTTCCCACGAAG
CAGCCGCGCAGTACGCGCCATCAACTGAGCGCAGTGGCCGCCCTT
CCCTCCCCCAGCTCCACGTTTTGTTTTTAAATGCAATAACTTATT
GCCTCTTTTCAGAAGGATGTGACAATATTACCAGCCCCCTCCCCC
TTCTGAATCTTAGGAGGTATGACCCAGAGCCACCATGGCTGCCTT
TCTGGGGAAGACCTAGAGTCCCTATGGTCCCTGGGGGCTGGTTCC
CCGGTGGCCCAGGTGGCCCAGGCAGGCCTGTGCCCTTGTGCCTTT
GTGCCTTCCTGCCAGCTGGAAGCAGTGTTTGGGGGGCCCTTGCCC
TCTTCCCACTGGGCTCCCTACCTGGGCCAAAGTCAACATCATTGC
TGGGGAAGAAGTGTTTTGGCTGTGTCAAAATAGTAGCTCCCAGAG
GAAGCAAGCCATGCTGGGAAAAAGGAAGTGGGTCAAAAAAGCGTA
GGGCTGCACTGTGATGTGAGGACCGCCATATGCAAGAGGCCTTTG
AGGGTCCAGGAGGATGGCCACCCTCGCTCTAGGCCGTAATGCATG
TGCTTAAATGCAAGACAAATGGAGCCATAGCCAGCCGTACCCAGC
CTGGCTCACCCTCCTCCAGACACCAGACACCAGCCTCCTGGTTGT
GGTGAGAGGAGAAGGGAACAGGTCTGGTGGATGCCAAGCAACTAG
TTAGTGCCTCCCAGTCTGACCTAGATTTGCATTCCTCATCAGGAC
TAGAAATTAGTACCAATTGAACTAGCTTGTTTTGACAGGTCTATT
TCACATCCTATGAATGTATGTAAATAAACTGTACATAGGTACGCA
TCTACATAAAATATCTTTTAATAACACGTTGACATTTGTGTAAAT
TTGAAATTTAAAAAAATTCTATAAAGTTGGTGTACATATGTTACA
ATTATGTATATTTTCTTTGGTCCTTCATAAAAAATATATTTACTT
TGCCAATAAAAAGAGGAAAAAAAAAAAGAACTC

In some embodiments, an oligonucleotide comprises a region of complementarity to a KLF15 sequence as set forth in SEQ ID NO: 740 or SEQ ID NO: 741. In some embodiments, the oligonucleotide comprises a region of complementarity that is at least 80%, 85%, 90%, 95%, 96%, 97%, 98%, 99%, or 100% complementary to a KLF15 sequence as set forth in SEQ ID NO: 740 or SEQ ID NO: 741. In some embodiments, the oligonucleotide comprises a sequence that has at least 10, 11, 12, 13, 14, 15, 16, 17, 18, or 19 consecutive nucleotides that are perfectly complementary to a KLF15 sequence as set forth in SEQ ID NO: 740 or SEQ ID NO: 741. In some embodiments, an oligonucleotide may comprise a sequence that targets (e.g., is complementary to) an RNA version (i.e., wherein the T's are replaced with U's) of a KLF15 sequence as set forth in SEQ ID NO: 740 or SEQ ID NO: 741. In some embodiments, the oligonucleotide comprises a sequence that is complementary (e.g., at least 80%, 85%, 90%, 95%, 96%, 97%, 98%, 99%, or 100% complementary) to an RNA version of a KLF15 sequence as set forth in SEQ ID NO: 740 or SEQ ID NO: 741. In some embodiments, the oligonucleotide comprises a sequence that has at least 10, 11, 12, 13, 14, 15, 16, 17, 18, or 19 consecutive nucleotides that are perfectly complementary to an RNA version of a KLF15 sequence as set forth in SEQ ID NO: 740 or SEQ ID NO: 741.

In some embodiments, a KLF15-targeting oligonucleotide comprises an antisense strand that comprises at least 10, 11, 12, 13, 14, 15, 16, 17, 18, or 19 consecutive nucleotides of a sequence comprising any one of SEQ ID NOs: 790-813. In some embodiments, a KLF15-targeting oligonucleotide comprises an antisense strand that comprises any one of SEQ ID NOs: 790-813. In some embodiments, a KLF15-targeting oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 790-813.

In some embodiments, a KLF15-targeting oligonucleotide comprises an antisense strand that targets a KLF15 sequence comprising any one of SEQ ID NOs: 742-789. In some embodiments, an oligonucleotide comprises an antisense strand comprising at least 10, 11, 12, 13, 14, 15, 16, 17, 18, or 19 nucleotides (e.g., consecutive nucleotides) that are complementary to a KLF15 sequence comprising any one of SEQ ID NOs: 742-789. In some embodiments, a KLF15-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NOs: 742-789.

In some embodiments, a KLF15-targeting oligonucleotide comprises an antisense strand comprises a region of complementarity to a target sequence as set forth in any one of SEQ ID NOs: 742-789. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a KLF15-targeting oligonucleotide further comprises a sense strand that hybridizes to the antisense strand to form a double stranded siRNA. In some embodiments, the KLF15-targeting oligonucleotide comprises an antisense strand that comprises the nucleotide sequence of any one of SEQ ID NOs: 790-813. In some embodiments, the KLF15-targeting oligonucleotide further comprises a sense strand that comprises the nucleotide sequence of any one of SEQ ID NOs: 766-789.

In some embodiments, the KLF15-targeing oligonucleotide is a double stranded oligonucleotide (e.g., an siRNA) comprising an antisense strand that comprises the nucleotide sequence of any one of SEQ ID NOs: 790-813 and a sense strand that hybridizes to the antisense strand and comprises the nucleotide sequence of any one of SEQ ID NOs: 766-789, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the KLF15-targeing oligonucleotide is a double stranded oligonucleotide (e.g., an siRNA) comprising an antisense strand that comprises the nucleotide sequence of any one of SEQ ID NOs: 790-813 and a sense strand that hybridizes to the antisense strand and comprises the nucleotide sequence of any one of SEQ ID NOs: 766-789, wherein the each nucleoside in the antisense strand and/or (e.g., and) each nucleoside in the sense strand is a 2′-modified nucleoside selected from 2′-O-Me and 2′-F modified nucleosides.

In some embodiments, the KLF15-targeing oligonucleotide is a double stranded oligonucleotide (e.g., an siRNA) comprising an antisense strand that comprises the nucleotide sequence of any one of SEQ ID NOs: 790-813 and a sense strand that hybridizes to the antisense strand and comprises the nucleotide sequence of any one of SEQ ID NOs: 766-789, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the KLF15-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the KLF15-targeing oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the KLF15-targeing oligonucleotide is selected from the modified version of SEQ ID NOs: 790-813 listed in Table 31. In some embodiments, the sense strand of the KLF15-targeing oligonucleotide is selected from the modified version of SEQ ID NOs: 766-789 listed in Table 31. In some embodiments, the KLF15-targeing oligonucleotide is a siRNA selected from the siRNAs listed in Table 31.

TABLE 29
KLF15 Target Sequences

| | Corresponding | | |
|---|---|---|---|
| | nucleotides of the | KLF15 Target Sequence | SEQ ID |
| Reference sequence | Reference Sequence | (5′ to 3′) | NO: |
| NM_014079.4 (SEQ | 1180-1199 | GCAGCAAGATGTACACCAA | 742 |
| ID NO: 740) | | | |
| NM_014079.4 (SEQ | 2350-2369 | AATGTATGTAAATAAACTG | 743 |
| ID NO: 740) | | | |
| NM_014079.4 (SEQ | 2352-2371 | TGTATGTAAATAAACTGTA | 744 |
| ID NO: 740) | | | |
| NM_014079.4 (SEQ | 2354-2372 | TATGTAAATAAACTGTACA | 745 |
| ID NO: 740) | | | |
| NM_014079.4 (SEQ | 2358-2377 | TAAATAAACTGTACATAGG | 746 |
| ID NO: 740) | | | |
| NM_014079.4 (SEQ | 2476-2495 | TATATTTTCTTTGGTCCTT | 747 |
| ID NO: 740) | | | |
| NM_014079.4 (SEQ | 2503-2522 | TATATTTACTTTGCCAATA | 748 |
| ID NO: 740) | | | |
| NM_014079.4 (SEQ | 240-259 | CCAAAATGCCCAGTTGGGT | 749 |
| ID NO: 740) | | | |
| NM_014079.4 (SEQ | 619-638 | TGGAGGAGATTGAAGAGTT | 750 |
| ID NO: 740) | | | |
| NM_014079.4 (SEQ | 1511-1530 | GCAATAATTTATTTGCCTC | 751 |
| ID NO: 740) | | | |
| NM_014079.4 (SEQ | 2379-2398 | CATCCACATAAAATATCTT | 752 |
| ID NO: 740) | | | |
| NM_014079.4 (SEQ | 688-707 | GCAAGGACTTGGATGCCTG | 753 |
| ID NO: 740) | | | |
| NM_014079.4 (SEQ | 1340-1359 | CCAGTGTCCTGTGTGCGAG | 754 |
| ID NO: 740) | | | |
| NM_014079.4 (SEQ | 1615-1634 | CGGGAGTACTTAGAGCCTC | 755 |
| ID NO: 740) | | | |
| NM_014079.4 (SEQ | 2330-2349 | GGTTTATTTCACATCCTAT | 756 |
| ID NO: 740) | | | |
| NM_023184.4 (SEQ | 1532-1551 | TCTGAATCTTAGGAGGTAT | 757 |
| ID NO: 741) | | | |
| NM_023184.4 (SEQ | 2204-2223 | CATCTACATAAAATATCTT | 758 |
| ID NO: 741) | | | |
| NM_023184.4 (SEQ | 1160-1179 | AAGATGTACACCAAGAGCA | 759 |
| ID NO: 741) | | | |
| NM_023184.4 (SEQ | 997-1016 | CTCAAAGTTTGTGCGAATT | 760 |
| ID NO: 741) | | | |
| NM_023184.4 (SEQ | 2118-2137 | GAAATTAGTACCAATTGAA | 761 |
| ID NO: 741) | | | |
| NM_023184.4 (SEQ | 393-412 | AGGGCAGCATCTTGGATTT | 762 |
| ID NO: 741) | | | |
| NM_023184.4 (SEQ | 2231-2250 | ACGTTGACATTTGTGTAAA | 763 |
| ID NO: 741) | | | |
| NM_023184.4 (SEQ | 2120-2139 | AATTAGTACCAATTGAACT | 764 |
| ID NO: 741) | | | |
| NM_023184.4 (SEQ | 263-282 | CAGCCATACCACATGTTGC | 765 |
| ID NO: 741) | | | |

* The target sequences contain Ts, but binding to RNA and/or DNA is contemplated.

In some embodiments, an oligonucleotide may comprise or consist of any sequence as provided in Table 30.

TABLE 30
Oligonucleotide sequences for targeting KLF15

| Passenger Strand/ | | Guide Strand/ | |
|---|---|---|---|
| Sense Strand | SEQ | Antisense Strand | SEQ |
| (RNA) | ID | (RNA) | ID |
| (5′ to 3′) | NO: | (5′ to 3′) | NO: |
| CUGCAGCAAGAUGUACACCAA | 766 | UUGGUGUACAUCUUGCUGCAGCC | 790 |
| UGAAUGUAUGUAAAUAAACUG | 767 | CAGUUUAUUUACAUACAUUCAUA | 791 |
| AAUGUAUGUAAAUAAACUGUA | 768 | UACAGUUUAUUUACAUACAUUCA | 792 |
| UGUAUGUAAAUAAACUGUACA | 769 | UGUACAGUUUAUUUACAUACAUU | 793 |
| UGUAAAUAAACUGUACAUAGG | 770 | CCUAUGUACAGUUUAUUUACAUA | 794 |
| CGUAUAUUUUCUUUGGUCCUU | 771 | AAGGACCAAAGAAAAUAUACGUA | 795 |
| AAUAUAUUUACUUUGCCAAUA | 772 | UAUUGGCAAAGUAAAUAUAUUUU | 796 |
| CGCCAAAAUGCCCAGUUGGGU | 773 | ACCCAACUGGGCAUUUUGGCGAC | 797 |
| CCUGGAGGAGAUUGAAGAGUU | 774 | AACUCUUCAAUCUCCUCCAGGGU | 798 |
| AAGCAAUAAUUUAUUUGCCUC | 775 | GAGGCAAAUAAAUUAUUGCUUAA | 799 |
| UCCAUCCACAUAAAAUAUCUU | 776 | AAGAUAUUUUAUGUGGAUGGACC | 800 |
| CAGCAAGGACUUGGAUGCCUG | 777 | CAGGCAUCCAAGUCCUUGCUGUU | 801 |
| UACCAGUGUCCUGUGUGCGAG | 778 | CUCGCACACAGGACACUGGUACG | 802 |
| CUCGGGAGUACUUAGAGCCUC | 779 | GAGGCUCUAAGUACUCCCGAGAA | 803 |
| CAGGUUUAUUUCACAUCCUAU | 780 | AUAGGAUGUGAAAUAAACCUGUC | 804 |
| CUUCUGAAUCUUAGGAGGUAU | 781 | AUACCUCCUAAGAUUCAGAAGGG | 805 |
| CGCAUCUACAUAAAAUAUCUU | 782 | AAGAUAUUUUAUGUAGAUGCGUA | 806 |
| GCAAGAUGUACACCAAGAGCA | 783 | UGCUCUUGGUGUACAUCUUGCUG | 807 |
| CCCUCAAAGUUUGUGCGAAUU | 784 | AAUUCGCACAAACUUUGAGGGCA | 808 |
| UAGAAAUUAGUACCAAUUGAA | 785 | UUCAAUUGGUACUAAUUUCUAGU | 809 |
| CCAGGGCAGCAUCUUGGAUUU | 786 | AAAUCCAAGAUGCUGCCCUGGGC | 810 |
| ACACGUUGACAUUUGUGUAAA | 787 | UUUACACAAAUGUCAACGUGUUA | 811 |
| GAAAUUAGUACCAAUUGAACU | 788 | AGUUCAAUUGGUACUAAUUUCUA | 812 |
| GGCAGCCAUACCACAUGUUGC | 789 | GCAACAUGUGGUAUGGCUGCCGG | 813 |

In some embodiments, an oligonucleotide is a modified oligonucleotide as provided in Table 31, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 31
Modified Oligonucleotides for targeting KLF15

| | Modified Passenger | SEQ | Modified Guide | SEQ |
|---|---|---|---|---|
| | Strand/Sense Strand (RNA) | ID | Strand/Antisense Strand (RNA) | ID |
| siRNA # | (5′ to 3′) | NO: | (5′ to 3′) | NO: |
| hsKLF15-1 | mCmUfGmCfAmGfCmAfAm | 766 | fUfUmGfGmUfGmUfAmCfAm | 790 |
| | GfAmUfGmUfAmCfAmCfC | | UfCmUfUmGfCmUfGmCfAmG | |
| | mAfA | | *fC*mC | |
| hsKLF15-2 | mUmGfAmAfUmGfUmAfU | 767 | fCfAmGfUmUfUmAfUmUfUm | 791 |
| | mGfUmAfAmAfUmAfAmAf | | AfCmAfUmAfCmAfUmUfCmA | |
| | CmUfG | | *fU*mA | |
| hsKLF15-3 | mAmAfUmGfUmAfUmGfU | 768 | fUfAmCfAmGfUmUfUmAfUm | 792 |
| | mAfAmAfUmAfAmAfCmUf | | UfUmAfCmAfUmAfCmAfUmU | |
| | GmUfA | | *fC*mA | |
| hsKLF15-4 | mUmGfUmAfUmGfUmAfA | 769 | fUfGmUfAmCfAmGfUmUfUm | 793 |
| | mAfUmAfAmAfCmUfGmUf | | AfUmUfUmAfCmAfUmAfCmA | |
| | AmCfA | | *fU*mU | |
| hsKLF15-5 | mUmGfUmAfAmAfUmAfA | 770 | fCfCmUfAmUfGmUfAmCfAmG | 794 |
| | mAfCmUfGmUfAmCfAmUf | | fUmUfUmAfUmUfUmAfCmA*f | |
| | AmGfG | | U*mA | |
| hsKLF15-6 | mCmGfUmAfUmAfUmUfU | 771 | fAfAmGfGmAfCmCfAmAfAmG | 795 |
| | mUfCmUfUmUfGmGfUmCf | | fAmAfAmAfUmAfUmAfCmG*f | |
| | CmUfU | | U*mA | |
| hsKLF15-7 | mAmAfUmAfUmAfUmUfU | 772 | fUfAmUfUmGfGmCfAmAfAm | 796 |
| | mAfCmUfUmUfGmCfCmAf | | GfUmAfAmAfUmAfUmAfUmU | |
| | AmUfA | | *fU*mU | |
| hsKLF15-8 | mCmGfCmCfAmAfAmAfUm | 773 | fAfCmCfCmAfAmCfUmGfGmG | 797 |
| | GfCmCfCmAfGmUfUmGfG | | fCmAfUmUfUmUfGmGfCmG*f | |
| | mGfU | | A*mC | |
| hsKLF15-9 | mCmCfUmGfGmAfGmGfAm | 774 | fAfAmCfUmCfUmUfCmAfAmU | 798 |
| | GfAmUfUmGfAmAfGmAfG | | fCmUfCmCfUmCfCmAfGmG*f | |
| | mUfU | | G*mU | |
| hsKLF15-10 | mAmAfGmCfAmAfUmAfA | 775 | fGfAmGfGmCfAmAfAmUfAm | 799 |
| | mUfUmUfAmUfUmUfGmCf | | AfAmUfUmAfUmUfGmCfUmU | |
| | CmUfC | | *fA*mA | |
| hsKLF15-11 | mUmCfCmAfUmCfCmAfCm | 776 | fAfAmGfAmUfAmUfUmUfUm | 800 |
| | AfUmAfAmAfAmUfAmUfC | | AfUmGfUmGfGmAfUmGfGmA | |
| | mUfU | | *fC*mC | |
| hsKLF15-12 | mCmAfGmCfAmAfGmGfAm | 777 | fCfAmGfGmCfAmUfCmCfAmA | 801 |
| | CfUmUfGmGfAmUfGmCfC | | fGmUfCmCfUmUfGmCfUmG*f | |
| | mUfG | | U*mU | |
| hsKLF15-13 | mUmAfCmCfAmGfUmGfUm | 778 | fCfUmCfGmCfAmCfAmCfAmG | 802 |
| | CfCmUfGmUfGmUfGmCfG | | fGmAfCmAfCmUfGmGfUmA*f | |
| | mAfG | | C*mG | |
| hsKLF15-14 | mCmUfCmGfGmGfAmGfUm | 779 | fGfAmGfGmCfUmCfUmAfAmG | 803 |
| | AfCmUfUmAfGmAfGmCfC | | fUmAfCmUfCmCfCmGfAmG*f | |
| | mUfC | | A*mA | |
| hsKLF15-15 | mCmAfGmGfUmUfUmAfU | 780 | fAfUmAfGmGfAmUfGmUfGm | 804 |
| | mUfUmCfAmCfAmUfCmCf | | AfAmAfUmAfAmAfCmCfUmG | |
| | UmAfU | | *fU*mC | |
| mmKLF15-1 | mCmUfUmCfUmGfAmAfUm | 781 | fAfUmAfCmCfUmCfCmUfAmA | 805 |
| | CfUmUfAmGfGmAfGmGfU | | fGmAfUmUfCmAfGmAfAmG*f | |
| | mAfU | | G*mG | |
| mmKLF15-2 | mCmGfCmAfUmCfUmAfCm | 782 | fAfAmGfAmUfAmUfUmUfUm | 806 |
| | AfUmAfAmAfAmUfAmUfC | | AfUmGfUmAfGmAfUmGfCmG | |
| | mUfU | | *fU*mA | |
| mmKLF15-3 | mGmCfAmAfGmAfUmGfU | 783 | fUfGmCfUmCfUmUfGmGfUmG | 807 |
| | mAfCmAfCmCfAmAfGmAf | | fUmAfCmAfUmCfUmUfGmC*f | |
| | GmCfA | | U*mG | |
| mmKLF15-3 | mCmCfCmUfCmAfAmAfGm | 784 | fAfAmUfUmCfGmCfAmCfAmA | 808 |
| | UfUmUfGmUfGmCfGmAfA | | fAmCfUmUfUmGfAmGfGmG*f | |
| | mUfU | | C*mA | |
| mmKLF15-5 | mUmAfGmAfAmAfUmUfA | 785 | fUfUmCfAmAfUmUfGmGfUm | 809 |
| | mGfUmAfCmCfAmAfUmUf | | AfCmUfAmAfUmUfUmCfUmA | |
| | GmAfA | | *fG*mU | |
| mmKLF15-6 | mCmCfAmGfGmGfCmAfGm | 786 | fAfAmAfUmCfCmAfAmGfAmU | 810 |
| | CfAmUfCmUfUmGfGmAfU | | fGmCfUmGfCmCfCmUfGmG*f | |
| | mUfU | | G*mC | |
| mmKLF15-7 | mAmCfAmCfGmUfUmGfAm | 787 | fUfUmUfAmCfAmCfAmAfAmU | 811 |
| | CfAmUfUmUfGmUfGmUfA | | fGmUfCmAfAmCfGmUfGmU*f | |
| | mAfA | | U*mA | |
| mmKLF15-8 | mGmAfAmAfUmUfAmGfU | 788 | fAfGmUfUmCfAmAfUmUfGm | 812 |
| | mAfCmCfAmAfUmUfGmAf | | GfUmAfCmUfAmAfUmUfUmC | |
| | AmCfU | | *fU*mA | |
| mmKLF15-9 | mGmGfCmAfGmCfCmAfUm | 789 | fGfCmAfAmCfAmUfGmUfGmG | 813 |
| | AfCmCfAmCfAmUfGmUfU | | fUmAfUmGfGmCfUmGfCmC*f | |
| | mGfC | | G*mG | |

j. MED1 Oligonucleotides

Examples of oligonucleotides useful for targeting MED1 are provided in Cai, Q. et. al. “MicroRNA-1291 mediates cell proliferation and tumorigenesis by downregulating MED1 in prostate cancer” Oncol Lett. 2019 March; 17(3):3253-3260.; Zhang, L. et. al. “Silencing MED1 Sensitizes Breast Cancer Cells to Pure Anti-Estrogen Fulvestrant In Vitro and In Vivo” PLoS One. 2013, 8(7): e70641.; Mouillet J. F. et. al. “MiR-205 silences MED1 in hypoxic primary human trophoblasts” FASEB J. 2010 June; 24(6):2030-9.; and Ndong, Jde. L. et al. “Down-regulation of the expression of RB18A/MED1, a cofactor of transcription, triggers strong tumorigenic phenotype of human melanoma cells” Int J Cancer. 2009, 124 (11):2597-606.; the contents of each of which are incorporated herein in their entireties.

In some embodiments, oligonucleotides may have a region of complementarity to a human MED1 sequence, for example, as provided below (Gene ID: 5469; NCBI Ref. No: NM_004774.4):

(SEQ ID NO: 814)
GAAGTTCCGTTGGGGAAGATGGCGGCGGCCTCGAGCACCCTTCTC
TTCTTGCCGCCGGGGACTTCAGATTGATCCTTCCCGGGAAGAGTA
GGGACTGCTGGTGCCCTGCGTCCCGGGATCCCGAGCCAACTTGTT
TCCTCCGTTAGTGGTGGGGAAGGGCTTATCCTTTTGTGGCGGATC
TAGCTTCTCCTCGCCTTCAGGATGAAAGCTCAGGGGGAAACCGAG
GAGTCAGAAAAGCTGAGTAAGATGAGTTCTCTCCTGGAACGGCTC
CATGCAAAATTTAACCAAAATAGACCCTGGAGTGAAACCATTAAG
CTTGTGCGTCAAGTCATGGAGAAGAGGGTTGTGATGAGTTCTGGA
GGGCATCAACATTTGGTCAGCTGTTTGGAGACATTGCAGAAGGCT
CTCAAAGTAACATCTTTACCAGCAATGACTGATCGTTTGGAGTCC
ATAGCAAGACAGAATGGACTGGGCTCTCATCTCAGTGCCAGTGGC
ACTGAATGTTACATCACGTCAGATATGTTCTATGTGGAAGTGCAG
TTAGATCCTGCAGGACAGCTTTGTGATGTAAAAGTGGCTCACCAT
GGGGAGAATCCTGTGAGCTGTCCGGAGCTTGTACAGCAGCTAAGG
GAAAAAAATTTTGATGAATTTTCTAAGCACCTTAAGGGCCTTGTT
AATCTGTATAACCTTCCAGGGGACAACAAACTGAAGACTAAAATG
TACTTGGCTCTCCAATCCTTAGAACAAGATCTTTCTAAAATGGCA
ATTATGTACTGGAAAGCAACTAATGCTGGTCCCTTGGATAAGATT
CTTCATGGAAGTGTTGGCTATCTCACACCAAGGAGTGGGGGTCAT
TTAATGAACCTGAAGTACTATGTCTCTCCTTCTGACCTACTGGAT
GACAAGACTGCATCTCCCATCATTTTGCATGAGAATAATGTTTCT
CGATCTTTGGGCATGAATGCATCAGTGACAATTGAAGGAACATCT
GCTGTGTACAAACTCCCAATTGCACCATTAATTATGGGGTCACAT
CCAGTTGACAATAAATGGACCCCTTCCTTCTCCTCAATCACCAGT
GCCAACAGTGTTGATCTTCCTGCCTGTTTCTTCTTGAAATTTCCC
CAGCCAATCCCAGTATCTAGAGCATTTGTTCAGAAACTGCAGAAC
TGCACAGGAATTCCATTGTTTGAAACTCAACCAACTTATGCACCC
CTGTATGAACTGATCACTCAGTTTGAGCTATCAAAGGACCCTGAC
CCCATACCTTTGAATCACAACATGAGATTTTATGCTGCTCTTCCT
GGTCAGCAGCACTGCTATTTCCTCAACAAGGATGCTCCTCTTCCA
GATGGCCGAAGTCTACAGGGAACCCTTGTTAGCAAAATCACCTTT
CAGCACCCTGGCCGAGTTCCTCTTATCCTAAATCTGATCAGACAC
CAAGTGGCCTATAACACCCTCATTGGAAGCTGTGTCAAAAGAACT
ATTCTGAAAGAAGATTCTCCTGGGCTTCTCCAATTTGAAGTGTGT
CCTCTCTCAGAGTCTCGTTTCAGCGTATCTTTTCAGCACCCTGTG
AATGACTCCCTGGTGTGTGTGGTAATGGATGTGCAGGACTCAACA
CATGTGAGCTGTAAACTCTACAAAGGGCTGTCGGATGCACTGATC
TGCACAGATGACTTCATTGCCAAAGTTGTTCAAAGATGTATGTCC
ATCCCTGTGACGATGAGGGCTATTCGGAGGAAAGCTGAAACCATT
CAAGCCGACACCCCAGCACTGTCCCTCATTGCAGAGACAGTTGAA
GACATGGTGAAAAAGAACCTGCCCCCGGCTAGCAGCCCAGGGTAT
GGCATGACCACAGGCAACAACCCAATGAGTGGTACCACTACACCA
ACCAACACCTTTCCGGGGGGTCCCATTACCACCTTGTTTAATATG
AGCATGAGCATCAAAGATCGGCATGAGTCGGTGGGCCATGGGGAG
GACTTCAGCAAGGTGTCTCAGAACCCAATTCTTACCAGTTTGTTG
CAAATCACAGGGAACGGGGGGTCTACCATTGGCTCGAGTCCGACC
CCTCCTCATCACACGCCGCCACCTGTCTCTTCGATGGCCGGCAAC
ACCAAGAACCACCCGATGCTCATGAACCTTCTTAAAGATAATCCT
GCCCAGGATTTCTCAACCCTTTATGGAAGCAGCCCTTTAGAAAGG
CAGAACTCCTCTTCCGGCTCACCCCGCATGGAAATATGCTCGGGG
AGCAACAAGACCAAGAAAAAGAAGTCATCAAGATTACCACCTGAG
AAACCAAAGCACCAGACTGAAGATGACTTTCAGAGGGAGCTATTT
TCAATGGATGTTGACTCACAGAACCCTATCTTTGATGTCAACATG
ACAGCTGACACGCTGGATACGCCACACATCACTCCAGCTCCAAGC
CAGTGTAGCACTCCCCCAACAACTTACCCACAACCAGTACCTCAC
CCCCAACCCAGTATTCAAAGGATGGTCCGACTATCCAGTTCAGAC
AGCATTGGCCCAGATGTAACTGACATCCTTTCAGACATTGCAGAA
GAAGCTTCTAAACTTCCCAGCACTAGTGATGATTGCCCAGCCATT
GGCACCCCTCTTCGAGATTCTTCAAGCTCTGGGCATTCTCAGAGT
ACCCTGTTTGACTCTGATGTCTTTCAAACTAACAATAATGAAAAT
CCATACACTGATCCAGCTGATCTTATTGCAGATGCTGCTGGAAGC
CCCAGTAGTGACTCTCCTACCAATCATTTTTTTCATGATGGAGTA
GATTTCAATCCTGATTTATTGAACAGCCAGAGCCAAAGTGGTTTT
GGAGAAGAATATTTTGATGAAAGCAGCCAAAGTGGGGATAATGAT
GATTTCAAAGGATTTGCATCTCAGGCACTAAATACTTTGGGGGTG
CCAATGCTTGGAGGTGATAATGGGGAGACCAAGTTTAAGGGCAAT
AACCAAGCCGACACAGTTGATTTCAGTATTATTTCAGTAGCCGGC
AAAGCTTTAGCTCCTGCAGATCTTATGGAGCATCACAGTGGTAGT
CAGGGTCCTTTACTGACCACTGGGGACTTAGGGAAAGAAAAGACT
CAAAAGAGGGTAAAGGAAGGCAATGGCACCAGTAATAGTACTCTC
TCGGGGCCCGGATTAGACAGCAAACCAGGGAAGCGCAGTCGGACC
CCTTCTAATGATGGGAAAAGCAAAGATAAGCCTCCAAAGCGGAAG
AAGGCAGACACTGAGGGAAAGTCTCCATCTCATAGTTCTTCTAAC
AGACCTTTTACCCCACCTACCAGTACAGGTGGATCTAAATCGCCA
GGCAGTGCAGGAAGATCTCAGACTCCCCCAGGTGTTGCCACACCA
CCCATTCCCAAAATCACTATTCAGATTCCTAAGGGAACAGTGATG
GTGGGCAAGCCTTCCTCTCACAGTCAGTATACCAGCAGTGGTTCT
GTGTCTTCCTCAGGCAGCAAAAGCCACCATAGCCATTCTTCCTCC
TCTTCCTCATCTGCTTCCACCTCAGGGAAGATGAAAAGCAGTAAA
TCAGAAGGTTCATCAAGTTCCAAGTTAAGTAGCAGTATGTATTCT
AGCCAGGGGTCTTCTGGATCTAGCCAGTCCAAAAATTCATCCCAG
TCTGGGGGGAAGCCAGGCTCCTCTCCCATAACCAAGCATGGACTG
AGCAGTGGCTCTAGCAGCACCAAGATGAAACCTCAAGGAAAGCCA
TCATCACTTATGAATCCTTCTTTAAGTAAACCAAACATATCCCCT
TCTCATTCAAGGCCACCTGGAGGCTCTGACAAGCTTGCCTCTCCA
ATGAAGCCTGTTCCTGGAACTCCTCCATCCTCTAAAGCCAAGTCC
CCTATCAGTTCAGGTTCTGGTGGTTCTCATATGTCTGGAACTAGT
TCAAGCTCTGGCATGAAGTCATCTTCAGGGTTAGGATCCTCAGGC
TCGTTGTCCCAGAAAACTCCCCCATCATCTAATTCCTGTACGGCA
TCTTCCTCCTCCTTTTCCTCAAGTGGCTCTTCCATGTCATCCTCT
CAGAACCAGCATGGGAGTTCTAAAGGAAAATCTCCCAGCAGAAAC
AAGAAGCCGTCCTTGACAGCTGTCATAGATAAACTGAAGCATGGG
GTTGTCACCAGTGGCCCTGGGGGTGAAGACCCACTGGACGGCCAG
ATGGGGGTGAGCACAAATTCTTCCAGCCATCCTATGTCCTCCAAA
CATAACATGTCAGGAGGAGAGTTTCAGGGCAAGCGTGAGAAAAGT
GATAAAGACAAATCAAAGGTTTCCACCTCCGGGAGTTCAGTGGAT
TCTTCTAAGAAGACCTCAGAGTCAAAAAATGTGGGGAGCACAGGT
GTGGCAAAAATTATCATCAGTAAGCATGATGGAGGCTCCCCTAGC
ATTAAAGCCAAAGTGACTTTGCAGAAACCTGGGGAAAGTAGTGGA
GAAGGGCTTAGGCCTCAAATGGCTTCTTCTAAAAACTATGGCTCT
CCACTCATCAGTGGTTCCACTCCAAAGCATGAGCGTGGCTCTCCC
AGCCATAGTAAGTCACCAGCATATACCCCCCAGAATCTGGACAGT
GAAAGTGAGTCAGGCTCCTCCATAGCAGAGAAATCTTATCAGAAT
AGTCCCAGCTCAGACGATGGTATCCGACCACTTCCAGAATACAGC
ACAGAGAAACATAAGAAGCACAAAAAGGAAAAGAAGAAAGTAAAA
GACAAAGATAGGGACCGAGACCGGGACAAAGACCGAGACAAGAAA
AAATCTCATAGCATCAAGCCAGAGAGTTGGTCCAAATCACCCATC
TCTTCAGACCAGTCCTTGTCTATGACAAGTAACACAATCTTATCT
GCAGACAGACCCTCAAGGCTCAGCCCAGACTTTATGATTGGGGAG
GAAGATGATGATCTTATGGATGTGGCCCTGATTGGGAATTAGGAA
CCTTATTTCCTAAAAGAAACAGGGCCAGAGGAAAAAAAACTATTG
ATAAGTTTATAGGCAAACCACCATAAGGGGTGAGTCAGACAGGTC
TGATTTGGTTAAGAATCCTAAATGGCATGGCTTTGACATCAAGCT
GGGTGAATTAGAAAGGCATATCCAGACCCTATTAAAGAAACCACA
GGGTTTGATTCTGGTTACCAGGAAGTCTTCTTTGTTCCTGTGCCA
GAAAGAAAGTTAAAATACTTGCTTAAGAAAGGGAGGGGGGTGGGA
GGGGTGTAGGGAGAGGGAAGGGAGGGAAACAGTTTTGTGGGAAAT
ATTCATATATATTTTCTTCTCCCTTTTTCCATTTTTAGGCCATGT
TTTAAACTCATTTTAGTGCATGTATATGAAGGGCTGGGCAGAAAA
TGAAAAAGCAATACATTCCTTGATGCATTTGCATGAAGGTTGTTC
AACTTTGTTTGAGGTAGTTGTCCGTTTGAGTCATGGGCAAATGAA
GGACTTTGGTCATTTTGGACACTTAAGTAATGTTTGGTGTCTGTT
TCTTAGGAGTGACTGGGGGAGGGAAGATTATTTTAGCTATTTATT
TGTAATATTTTAACCCTTTATCTGTTTGTTTTTATACAGTGTTTC
GTTCTAAATCTATGAGGTTTAGGGTTCAAAATGATGGAAGGCCGA
AGAGCAAGGCTTATATGGTGGTAGGGAGCTTATAGCTTGTGCTAA
TACTGTAGCATCAAGCCCAAGCAAATTAGTCAGAGCCCGCCTTTA
GAGTTAAATATAATAGAAAAACCAAAATGATATTTTTATTTTAGG
AGGGTTTAAATAGGGTTCAGAGATCATAGGAATATTAGGAGTTAC
CTCTCTGTGGAGGTATTGACTTGTAATCTCATTTTCCTTTCAAAA
AAAAAAAAAAAAGCTAAGGTGGCTTGTTGGGATGTAAACATGTTT
TCAGATGCAGTAAGGTTTAGTGTAGGACAGCCTTCCTGACCCAGT
GGCATGAAAACCATTACAGGATTAATAGTTCTCCTACTTCCACAA
TGTGCCAAAAGTCTGCATCCCAGCATTTTGTTTGCAGGAGAACTG
ATGCCATTCCTAAGAGCTGGACTCACTGTTTCTCTTCATCACAAG
GAGAAGGAGTCCAAACTTTAATCACTCCACTGTATGCTCCCTGAG
ATAAAACAGTAAAAAATCCGCAGCCATAGTTCACTTAAAACAATT
TCAAGCTCACTTTTGAAGTAATGGGGGCCTGGAATGCTAGGTGAG
CATGAAGATAAACCCTTGCTACTATGTAGCAACCCAATTGGACCT
TTTTGGAGAAATAGGTCTGAGTCTGGATTCTGGGGGACATCAATA
AGAGCCCTTCACATAAAAATATAGAAATCCAGGAGACTGTTTCGA
GTGCAACAGAAGTTCTCAGTATTTGGAGGGTCCTCTCAAAAATTC
TGCGGCCTTACTTTGATATTGACACCTGCACTGTGCCATTCCTGA
TTATTCCATTCAGGATCTGTATCAGCGGGATGGGGCATGGTCCCC
AGCACAACTCTTCTGGGTTAAAAAAAAAAAGCCAGGTGATTCCTT
TGTGTGTTATGTCGTAAGTGGAGTGACTTCATCATATATGGAAGA
AGATTTCTATATTCAGCTTTTTCTGCAGGTTGGAGTCAGCATAGA
GTTGGAAAATCAGCTTTGGCTTTCTTTCCTGTCTCATTTCCTCTA
GTGTTCTCCTTTTTATTGTCATCAGCTCTCAACAACTCTGCCACT
TTTGTGTCCCAAGGTAATAAGATGTAGGAAACAAAACATTGTAAA
GTGGAGCAAGAAAAGTTATCAATTAACCACATCAGAGTCAAATGT
CTTGGGTGACACTAAGGAGGATATGGGCAGGTGATACCAGAGTGC
TTTATCTTGTGATGTTGATACAGTAGCAGCCTCTCAGACATTCAG
CCAGGTTGGATTTCTCATGAGTTTGTCACCTAGTTTTGAATCCTA
TCCCGTTGGTTTCTGCAGGAAAAAAAAAAAAATTTATGTGGTTTT
TAAAATTTGTTCTGAGTGGGGAGAATCTTAGGGGGAATGTACTGA
ATAGTATCATGGGCTCAGCTCCCCCATGCAGGGCCAACAAATACC
AAAATGAGTAAACTGGGAAGCTTTTCTCTCTTTCTGTCTTCATCC
CAGATCAAAGAATCCCGAGTTAGGATCTGGATGAAGGATAAGCCC
CTGAATTGTCGATGGGCTCACCCCCACACTGACCCAGCATCTGAA
CTTGCTTAACAGGGAGCCGGGGCTAAACTGCTTCACCCTGCCTGA
GAACCAGGGAGCACTGCATTTCTCCACAGGGTGGAGGAGAAGAGG
CAGAATAAACCAAGCCTGGGACACCTCCCTCCTGTCTAGGTGTAC
TCATTCTTCTGTTTCAAAAGAAGGCAAGGACATGAAGTCAACTTC
TACCTATCTTCTGCTGCTGGTGTCTTATGTATTCTCAGTTTGACC
TGATTCCTCTTCTGTCTTTTGACTTAACATTAGGGGTTCTTGGTC
ATAACCTGCTCTGATGTACATAAAGATTTCAGGTTCAATATCAAT
GTGTCTTAAAACAGAAGTATTTTAGCGGGTGGGGGGTGGGGTGGT
GGGGACAAACAACGCAGGATATAATTGCCAAAACCAGGCTTGAGG
TTGGTGACTCTTGAAAGATTTTCTTTCTTCAGGCCTAGATCAGAA
AATTAAGTGCAGCAATATCATGAATTCTCAGAAGCCCTTTCAGGG
AGCCAGTGAGTCATACAGTATCCACAGTTGAGTCACTTAAAGATG
TCAGTATACGAAACATTATTCACAATCCTTGGGCAATCTCATTTT
TTTTTCCTTCTCCCCTCCTCCCCTGCCCCCCATACATTTCTATCC
TTGAGTTAGTTTTGGAGGGGCAGGAAGTACTTAACATCTCAGAAG
CTAGATTGGGAAACATGCTCAGCTATAAGAACTGAGCTTTAAATT
TTGAGTTTAAAAATGTACATCAGGAGCAGCTGGGGAGGGTCTTTT
TTTAAAAAAATCTTTCAAATTTGGTTTTCTGTGCATATGGCCGTT
TTGTAAATACTTTGGGGTTTTTCATTTTTTTGAAAGTAGATGAAA
TCTGTTGTGGGATTTTTTTCCCGAAACATTACAAAATAACCTGTT
TATTTACATGCAAATAAACTTCTTTGATAAAAAGTAA

In some embodiments, oligonucleotides may have a region of complementarity to a mouse MED1 sequence, for example, as provided below by Gene ID: 19014; NCBI Ref. No: NM_001080118.1:

(SEQ ID NO: 815)
GATCTTTGGGAAGTTCCGTTGGGGAAGATGGCGGCGGCCGCGAGC
ACCCTTCTCTTCTTGCCGCCGGGGACTTCAGACTGAGCCTTCCCC
GGAAGAGTCGGGGCCGCTGGTGCGCCGCTTCCCCGGAGCCCAGGC
TAACTTGGTTCCCCTCTGAGTGGCGGGGAAAGGCTCCCCGGCTCG
TGGCGGACCCAAGACCTCCCCGCTGTCAGGATGAAGGCTCAGGGG
GAAACCGAGGACTCAGAGAGGCTGAGTAAGATGAGCTCCCTCCTG
GAACGGCTCCATGCAAAATTTAACCAGAACAGACCTTGGAGTGAA
ACCATTAAGCTTGTGCGTCAAGTAATGGAGAAGAGGGTCGTAATG
AGTTCTGGAGGGCATCAGCATTTGGTCAGCTGTTTGGAGACATTG
CAGAAGGCTCTCAAAGTAACATCTTTGCCAGCAATGACTGATCGT
TTGGAATCTATAGCCAGACAGAATGGACTGGGCTCTCACCTCAGT
GCCAGTGGCACTGAGTGTTACATCACGTCAGATATGTTCTATGTG
GAAGTGCAGTTAGATCCTGCAGGACAGCTTTGTGATGTCAAAGTG
GCTCACCATGGGGAGAATCCTGTGAGCTGTCCAGAGCTTGTACAG
CAGTTAAGGGAAAAGAATTTTGAGGAATTTTCCAAGCATCTTAAG
GGTCTTGTTAATCTGTATAATCTCCCAGGGGACAACAAACTGAAG
ACTAAAATGTATCTGGCTCTCCAATCCTTAGAACAGGACCTTTCT
AAAATGGCTATTATGTACTGGAAGGCAACCAACGCCGCTCCCTTG
GATAAGATTCTTCATGGAAGTGTTGGTTATCTCACCCCGCGGAGT
GGGGGTCATTTAATGAATATGAAATACTATGCCTCTCCATCTGAC
CTGCTGGATGATAAGACTGCCTCTCCTATCATTTTGCATGAAAAG
AATGTTCCTCGGTCTTTGGGAATGAATGCCTCAGTGACAATTGAA
GGAACCTCTGCTATGTACAAACTCCCAATTGCCCCATTAATTATG
GGGTCACACCCAGCTGACAACAAATGGACCCCTTCTTTCTCCGCA
GTCACTAGTGCCAACAGTGTTGATCTTCCTGCGTGTTTCTTCTTG
AAATTTCCCCAGCCAATTCCAGTATCTAAAGCATTTGTTCAGAAA
CTGCAAAATTGCACAGGAATCCCGTTGTTTGAGACTCCGCCCACT
TACCTGCCCCTGTATGAACTCATCACTCAGTTTGAGCTGTCAAAG
GATCCTGACCCTTTACCTTTGAATCACAACATGCGATTTTACGCT
GCTCTTCCAGGTCAGCAGCACTGCTATTTTCTCAATAAAGATGCT
CCTCTTCCTGATGGTCAGAGCCTGCAGGGAACACTGGTCAGCAAA
ATCACCTTCCAGCACCCTGGCCGAGTTCCTCTTATCTTGAATATG
ATCAGACACCAAGTGGCCTATAACACTCTAATTGGAAGCTGTGTC
AAAAGAACTATTTTAAAAGAAGATTCTCCTGGGCTCCTCCAGTTT
GAAGTGTGTCCTCTCTCAGAATCTCGCTTCAGTGTATCTTTTCAG
CACCCTGTGAATGACTCCCTTGTGTGTGTGGTGATGGATGTGCAA
GACTCAACACATGTGAGCTGTAAACTCTACAAGGGGCTGTCAGAT
GCACTAATCTGTACAGACGACTTCATTGCCAAAGTTGTTCAAAGA
TGTATGTCCATTCCTGTGACGATGAGGGCTATTCGGAGGAAGGCT
GAAACCATACAGGCTGACACCCCAGCACTGTCTCTCATTGCAGAG
ACAGTTGAAGACATGGTGAAAAAGAACCTGCCCCCGGCTAGCAGC
CCAGGGTATGGCATGACCACAGGCAACAACCCAATGAGTGGTACC
ACTACACCAACCAACACCTTTCCGGGGGGTCCCATTACCACCTTG
TTTAATATGAGCATGAGCATCAAAGATCGGCATGAGTCGGTGGGC
CATGGGGAGGACTTCAGCAAGGTGTCTCAGAACCCAATTCTTACC
AGTTTGTTGCAAATCACAGGGAACGGGGGGTCTACCATTGGCTCG
AGTCCGACCCCTCCTCATCACACGCCGCCACCTGTCTCTTCGATG
GCCGGCAACACCAAGAACCACCCGATGCTCATGAACCTTCTTAAA
GATAACCCTGCCCAGGATTTCTCAACCCTTTATGGAAGCAGCCCT
TTAGAAAGGCAGAACTCCTCTTCCGGATCACCCCGGATGGAAATG
TGCTCGGGGAGCAACAAGGCCAAGAAGAAGAAGTCGTCAAGAGTC
CCACCTGACAAACCCAAGCACCAGACTGAAGACGATTTCCAGAGG
GAGCTCTTTTCCATGGATGTCGACTCACAGAACCCTATGTTTGAC
GTCAGCATGACCGCTGACGCGCTGGATACACCTCATATCACCCCA
GCTCCAAGCCAGTGTAGCACTCCCCCAGCAACGTACCCACAGCCA
GTGTCTCACCCCCAGCCCAGTATTCAGAGGATGGTCCGACTGTCC
AGTTCAGACAGCATTGGCCCAGATGTAACTGATATTCTTTCAGAT
ATTGCCGAAGAAGCTTCAAAGCTTCCCAGCACGAGTGATGACTGC
CCACCAATTGGCACCCCTGTTCGAGATTCCTCAAGTTCTGGGCAT
TCTCAGAGTGCCCTCTTTGATTCTGATGTCTTTCAAACTAATAAT
AATGAAAATCCATACACTGATCCAGCTGACCTTATTGCAGATGCT
GCTGGAAGCCCCAATAGTGATTCTCCTACCAATCATTTTTTCCCT
GATGGAGTAGATTTCAATCCTGATTTGTTGAACAGCCAAAGCCAA
AGTGGTTTTGGAGAGGAGTATTTTGATGAAAGTAGTCAGAGTGGG
GATAATGATGATTTCAAAGGATTTGCATCTCAGGCATTAAATACA
TTGGGGATGCCAATGCTTGGAGGTGACAATGGGGAGCCAAAATTT
AAGGGCAGCAGCCAGGCTGACACGGTGGACTTCAGTATTATATCA
GTAGCCGGTAAGGCTTTGGGTGCTGCAGATCTGATGGAGCACCAC
AGTGGGAGTCAGAGTCCTTTACTGACCACTGGAGAATTAGGGAAA
GAAAAAACTCAAAAGAGGGTGAAGGAAGGCAACGGCACAGGTGCT
AGCAGTGGATCAGGTCCAGGGTCAGACAGCAAGCCAGGCAAGCGC
AGCCGCACTCCCTCCAATGATGGGAAGAGCAAGGATAAGCCTCCA
AAGCGGAAGAAGGCAGACACTGAGGGGAAGTCCCCATCTCACAGT
TCTTCTAATAGACCTTTCACCCCACCTACCAGCACGGGTGGGTCC
AAATCCCCAGGCAGTTCAGGACGATCTCAGACGCCCCCAGGTGTT
GCCACCCCGCCCATTCCCAAGATTACCATTCAGATTCCTAAAGGG
ACAGTGATGGTGGGCAAGCCCTCCTCTCACAGTCAGTACACTAGC
AGTGGTTCTGTGTCTTCCTCTGGCAGCAAAAGCCACCATAGTCAT
TCTTCCTCCTCCTCTTCCTTAGCTTCTGCTTCCACCTCAGGCAAG
GTGAAAAGCAGTAAATCTGAAGGCTCATCAAGTTCCAAGCTCAGT
GGCAGTATGTATGCTAGCCAAGGGTCTTCTGGATCCAGCCAGTCC
AAAAATTCATCTCAGACTGGGGGGAAGCCAGGCTCCTCTCCCATT
ACCAAACATGGACTGAGCAGTGGGTCCAGCAGTACCAAGATGAAA
CCTCAAGGCAAGCCATCCTCCCTTATGAACCCTTCTATAAGTAAG
CCAAACATATCCCCTTCCCATTCAAGGCCTCCCGGAGGCTCAGAT
AAGCTTGCCTCTCCAATGAAGCCTGTTCCTGGAACCCCCCCATCC
TCTAAAGCCAAGTCCCCTATCAGTTCAGGTTCCAGTGGTTCTCAT
GTGTCAGGAACTAGTTCAAGCTCTGGTATGAAGTCATCTTCAGGG
TCAGCATCCTCAGGCTCAGTGTCTCAAAAAACCCCTCCAGCATCT
AATTCTTGTACACCATCTTCCTCTTCGTTTTCCTCAAGTGGTTCT
TCCATGTCATCCTCTCAGAATCAACATGGCAGTTCCAAAGGGAAA
TCTCCCAGTAGGAATAAGAAGCCTTCCTTGACAGCTGTCATAGAT
AAATTGAAGCATGGGGTGGTTACCAGTGGGCCTGGGGGTGAGGAT
CCAATAGACAGTCAGATGGGCGCAAGCACAAATTCTTCTAACCAT
CCCATGTCCTCCAAACATAACACGTCAGGAGGGGAGTTCCAGAGC
AAACGTGAGAAAAGTGATAAAGACAAATCCAAGGTCTCTGCTTCT
GGGGGGTCAGTGGATTCCTCTAAGAAGACTTCAGAGTCAAAAAAT
GTGGGGAGCACGGGGGTGGCAAAAATCATTATCAGCAAGCACGAC
GGAGGCTCCCCGAGCATCAAAGCCAAAGTGACGCTACAGAAACCT
GGAGAAAGTGGTGGAGATGGGCTCAGGCCACAGATAGCCTCATCA
AAGAACTATGGCTCTCCACTTATCAGTGGTTCCACTCCAAAGCAC
GAACGGGGTTCTCCCAGCCACAGTAAGTCGCCAGCATATACACCA
CAGAATGTGGACAGTGAAAGTGAGTCAGGCTCCTCCATAGCAGAG
AGATCCTACCAGAACAGTCCCAGCTCAGAGGATGGTATCCGACCA
CTTCCAGAGTACAGCACTGAGAAGCATAAGAAGCACAAAAAGGAA
AAGAAGAAAGTCAGAGACAAAGACAGAGACAAGAAGAAGTCTCAC
AGCATGAAGCCAGAGAACTGGTCGAAATCCCCCATTTCTTCAGAT
CCGACGGCGTCTGTGACAAATAACCCTATCTTATCTGCAGACAGG
CCTTCTAGGCTCAGCCCTGACTTCATGATTGGGGAGGAAGATGAT
GATCTCATGGATGTGGCCCTGATTGGCAATTAGCCTAACTTTCTA
AACAGACACGTCCAGAGGAGAAACTAATGATATATGTAAACCAAC
CCGAGGGGTGAGTCAGACAGGCCCTGTCAGTGCTTAAGAGCCCTA
AAGGGCATGGCCTCTACACCAAGCTGGGTACATTTATAAACATAC
ATCTAGACCCTGCTGCCGAAACCATGTGGTTGGTTGTTCAGAAAC
AAGTCATATTTCTTTGCCAGAAAGCAATGAATAAATTACTTGCTT
AAGGGGAGAGGGGTGGTGGGAGAGGGTGTAGAGAGGGAGGGGTGG
GAAATTGTTGTGGGAAATATTCATATATTTTTCTCCCTTTTTCCA
TTTTCAGGACATGCTTTAAACTCATTTTAGTGCATGTATTTGAAG
GGCTGGGCAGAAAATGAAAAAGCAATACATTCCTTGATGCATTCG
CATGAAGGTTGCTCACCTGTGGGAGATGCCCATTGGAAGCATGGG
CACGGGAAGGACTTTGACAGTTCTGGACACTGAAAGCAATGTTTG
GTGTCTGTTTCTTAGGAGTGATGGGGGAGGGAAAATTATTTTAGT
TATTTATTTGTAATATTTTAATCCTTGATGTTTGTTTTTATGCAA
TGTTTGTTCCTAAATCTATGAAGGTTATGGTTCAGAACAGTAGGA
GGCAGGTAGCAAGGCTCGCCTGGTGGGGAGCTCGCAGCCTGTGCT
GACGCTGCAGCACCAGTCCCACAGAGCTAATCACAGCCTGTCTTT
GGAGTTGAATAGAAAAACCAAAACTATTATCTTTGGAGGTTTTTC
ACAGGGTTCAGACACCATAGGAATATTAGGAGTGAGTTACCTCCC
TATGCAGATAAAAGATTTATCTTCTTTCATTAAAAAGGAAGGGTG
GCTTTGGGGGGTATACATTTGTTTTCAGAGTCTATACCCTATCGT
ATAAAAATCAGTATAGAATTAATAGTTCTCCTACTTCCACAATGT
GCCAAAAGTCTACATCCCAGCATTTTGTTTGCAGGAGAGCTGATG
CCATTCCTAAGAGCTGGACTCTTTGTTTCTCTTCATCACAAGAAG
AAGGAGTTCAGACTTAAATCACTCCACTGTATGCTCCCCGAGATA
AACAGTTAAAAAAAAAAAAAGCAGCCATAGTTCACTTAAAACAAT
TTCAAGCTCACTTGAAGTGATTAGGGCCTGGAATGCTAAGTGAGC
ATGAAGATAACCCTTGCTACCGTGTAGCACACAGTCGGACCTTTT
TGGAGAAACAGGTCTGAGTCTGGATTCTGAGGGACATCAAAGGAA
GACCTCATAAATAGAGAAATGCAGGAGACCGTTTTGAGTGCAACA
GAAGTTCTCAGTATTTGGAGGGTCCTCTCAAAAGTCCGCGGCCTT
ACTTTGATTTCACCACCTGCACTGTGCCATTCCTGATGATTCCGT
TTGGGATCTGTATCAGCGGGGTGGGGGTGGGGCGTGATCCCCAGC
ACAACTCTTCTGGATTACAAAACAAGCAAAAGCCAGGTGATTCAT
GTTTGTGTGTGTTATAAGTGGAGTGACTTCATCAGATACGGACGA
TTTCTACATCCAACACTGCAGGTTGTTGGAGTTAGCAT

In some embodiments, the oligonucleotide may have region of complementarity to a mutant form of MED1.

In some embodiments, an oligonucleotide comprises a region of complementarity to a MED1 sequence as set forth in SEQ ID NO: 814 or SEQ ID NO: 815. In some embodiments, the oligonucleotide comprises a region of complementarity that is at least 80%, 85%, 90%, 95%, 96%, 97%, 98%, 99%, or 100% complementary to an a MED1 sequence as set forth in SEQ ID NO: 814 or SEQ ID NO: 815. In some embodiments, the oligonucleotide comprises a sequence that has at least 10, 11, 12, 13, 14, 15, 16, 17, 18, or 19 consecutive nucleotides that are perfectly complementary to a MED1 sequence as set forth in SEQ ID NO: 814 or SEQ ID NO: 815. In some embodiments, an oligonucleotide may comprise a sequence that targets (e.g., is complementary to) an RNA version (i.e., wherein the T's are replaced with U's) of a MED1 sequence as set forth in SEQ ID NO: 814 or SEQ ID NO: 815. In some embodiments, the oligonucleotide comprises a sequence that is complementary (e.g., at least 80%, 85%, 90%, 95%, 96%, 97%, 98%, 99%, or 100% complementary) to an RNA version of a MED1 sequence as set forth in SEQ ID NO: 814 or SEQ ID NO: 815. In some embodiments, the oligonucleotide comprises a sequence that has at least 10, 11, 12, 13, 14, 15, 16, 17, 18, or 19 consecutive nucleotides that are perfectly complementary to an RNA version of a MED1 sequence as set forth in SEQ ID NO: 814 or SEQ ID NO: 815.

In some embodiments, a MED1-targeting oligonucleotide comprises an antisense strand that comprises at least 10, 11, 12, 13, 14, 15, 16, 17, 18, or 19 consecutive nucleotides of a sequence comprising any one of SEQ ID NOs: 864-887. In some embodiments, a MED1-targeting oligonucleotide comprises an antisense strand that comprises any one of SEQ ID NOs: 864-887. In some embodiments, a MED1-targeting oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 864-887.

In some embodiments, a MED1-targeting oligonucleotide comprises an antisense strand that targets a MED1 sequence comprising any one of SEQ ID NOs: 816-863. In some embodiments, an oligonucleotide comprises an antisense strand comprising at least 10, 11, 12, 13, 14, 15, 16, 17, 18, or 19 nucleotides (e.g., consecutive nucleotides) that are complementary to a MED1 sequence comprising any one of SEQ ID NOs: 816-863. In some embodiments, a MED1-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NOs: 816-863.

In some embodiments, a MED1-targeting oligonucleotide comprises an antisense strand comprises a region of complementarity to a target sequence as set forth in any one of SEQ ID NOs: 816-863. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a MED1-targeting oligonucleotide further comprises a sense strand that hybridizes to the antisense strand to form a double stranded siRNA. In some embodiments, the MED1-targeting oligonucleotide comprises an antisense strand that comprises the nucleotide sequence of any one of SEQ ID NOs: 864-887. In some embodiments, the MED1-targeting oligonucleotide further comprises a sense strand that comprises the nucleotide sequence of any one of SEQ ID NOs: 840-863.

In some embodiments, the MED1-targeing oligonucleotide is a double stranded oligonucleotide (e.g., an siRNA) comprising an antisense strand that comprises the nucleotide sequence of any one of SEQ ID NOs: 864-887 and a sense strand that hybridizes to the antisense strand and comprises the nucleotide sequence of any one of SEQ ID NOs: 840-863, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the MED1-targeing oligonucleotide is a double stranded oligonucleotide (e.g., an siRNA) comprising an antisense strand that comprises the nucleotide sequence of any one of SEQ ID NOs: 864-887 and a sense strand that hybridizes to the antisense strand and comprises the nucleotide sequence of any one of SEQ ID NOs: 840-863, wherein the each nucleoside in the antisense strand and/or (e.g., and) each nucleoside in the sense strand is a 2′-modified nucleoside selected from 2′-O-Me and 2′-F modified nucleosides.

In some embodiments, the MED1-targeing oligonucleotide is a double stranded oligonucleotide (e.g., an siRNA) comprising an antisense strand that comprises the nucleotide sequence of any one of SEQ ID NOs: 864-887 and a sense strand that hybridizes to the antisense strand and comprises the nucleotide sequence of any one of SEQ ID NOs: 840-863, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the MED1-targeing oligonucleotide comprises a structure of (5′ to 3′):
fNfNmNfNmNfNmNfNmNfNmNfNmNfNmNfNmNfNmNfNmN*fN*mN, wherein “IN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the MED1-targeing oligonucleotide comprises a structure of (5′ to 3′):
mNmNfNmNfNmNfNmNfNmNfNmNfNmNfNmNfNmNfNmNfN, wherein “inN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the MED1-targeing oligonucleotide is selected from the modified version of SEQ ID NOs: 864-887 listed in Table 34. In some embodiments, the sense strand of the MED1-targeing oligonucleotide is selected from the modified version of SEQ ID NOs: 840-863 listed in Table 34. In some embodiments, the MED1-targeing oligonucleotide is a siRNA selected from the siRNAs listed in Table 34.

TABLE 32
MED1 Target Sequences

| Reference | Corresponding | | |
|---|---|---|---|
| sequence | nucleotides of | MED1 Target | |
| NM_004774.4 | the Reference | Sequence | SEQ ID |
| (SEQ ID NO: 814) | Sequence | (5′ to 3′) | NO: |
| NM_004774.4 | 503-521 | GTTACATCACGTCAGATAT | 816 |
| NM_004774.4 | 508-526 | ATCACGTCAGATATGTTCT | 817 |
| NM_004774.4 | 515-533 | CAGATATGTTCTATGTGGA | 818 |
| NM_004774.4 | 1432-1450 | ATCAGACACCAAGTGGCCT | 819 |
| NM_004774.4 | 1676-1692 | ACTTCATTGCCAAAGTTGT | 820 |
| NM_004774.4 | 1688-1706 | AAGTTGTTCAAAGATGTAT | 821 |
| NM_004774.4 | 1693-1711 | GTTCAAAGATGTATGTCCA | 822 |
| NM_004774.4 | 1837-1855 | CCAGGGTATGGCATGACCA | 823 |
| NM_004774.4 | 1923-1941 | CTTGTTTAATATGAGCATG | 824 |
| NM_004774.4 | 2126-2144 | ACCCGATGCTCATGAACCT | 825 |
| NM_004774.4 | 2786-2804 | GAGTAGATTTCAATCCTGA | 826 |
| NM_004774.4 | 3454-3472 | AGCAGTGGTTCTGTGTCTT | 827 |
| NM_004774.4 | 2005-2023 | CCAATTCTTACCAGTTTGT | 828 |
| NM_004774.4 | 1978-1996 | GAGGACTTCAGCAAGGTGT | 829 |
| NM_004774.4 | 4601-4619 | CAGGCTCCTCCATAGCAGA | 830 |
| NM_004774.4 | 5482-5500 | GTGTCTGTTTCTTAGGAGT | 831 |
| NM_004774.4 | 400-418 | AAGGCTCTCAAAGTAACAT | 832 |
| NM_004774.4 | 1940-1958 | TGAGCATCAAAGATCGGCA | 833 |
| NM_004774.4 | 5363-5381 | GCAATACATTCCTTGATGC | 834 |
| NM_004774.4 | 6157-6175 | AAAACAATTTCAAGCTCAC | 835 |
| NM_004774.4 | 1937-1955 | GCATGAGCATCAAAGATCG | 836 |
| NM_004774.4 | 995-1013 | TGTACAAACTCCCAATTGC | 837 |
| NM_004774.4 | 4600-4618 | TCAGGCTCCTCCATAGCAG | 838 |
| NM_004774.4 | 5483-5501 | TGTCTGTTTCTTAGGAGTG | 839 |

* The target sequences contain Ts, but binding to RNA and/or DNA is contemplated.

In some embodiments, an oligonucleotide may comprise or consist of any sequence as provided in Table 33.

TABLE 33
Oligonucleotide sequences for targeting MED1

| Passenger Strand/ | | Guide Strand/ | |
|---|---|---|---|
| Sense Strand | SEQ | Antisense Strand | SEQ |
| (RNA) | ID | (RNA) | ID |
| (5′ to 3′) | NO: | (5′ to 3′) | NO: |
| AUGUUACAUCACGUCAGAUAU | 840 | AUAUCUGACGUGAUGUAACAUUC | 864 |
| ACAUCACGUCAGAUAUGUUCU | 841 | AGAACAUAUCUGACGUGAUGUAA | 865 |
| GUCAGAUAUGUUCUAUGUGGA | 842 | UCCACAUAGAACAUAUCUGACGU | 866 |
| UGAUCAGACACCAAGUGGCCU | 843 | AGGCCACUUGGUGUCUGAUCAGA | 867 |
| UGACUUCAUUGCCAAAGUUGU | 844 | ACAACUUUGGCAAUGAAGUCAUC | 868 |
| CAAAGUUGUUCAAAGAUGUAU | 845 | AUACAUCUUUGAACAACUUUGGC | 869 |
| UUGUUCAAAGAUGUAUGUCCA | 846 | UGGACAUACAUCUUUGAACAACU | 870 |
| GCCCAGGGUAUGGCAUGACCA | 847 | UGGUCAUGCCAUACCCUGGGCUG | 871 |
| ACCUUGUUUAAUAUGAGCAUG | 848 | CAUGCUCAUAUUAAACAAGGUGG | 872 |
| CCACCCGAUGCUCAUGAACCU | 849 | AGGUUCAUGAGCAUCGGGUGGUU | 873 |
| UGGAGUAGAUUUCAAUCCUGA | 850 | UCAGGAUUGAAAUCUACUCCAUC | 874 |
| CCAGCAGUGGUUCUGUGUCUU | 851 | AAGACACAGAACCACUGCUGGUA | 875 |
| ACCCAAUUCUUACCAGUUUGU | 852 | ACAAACUGGUAAGAAUUGGGUUC | 876 |
| GGGAGGACUUCAGCAAGGUGU | 853 | ACACCUUGCUGAAGUCCUCCCCA | 877 |
| GUCAGGCUCCUCCAUAGCAGA | 854 | UCUGCUAUGGAGGAGCCUGACUC | 878 |
| UGGUGUCUGUUUCUUAGGAGU | 855 | ACUCCUAAGAAACAGACACCAAA | 879 |
| AGAAGGCUCUCAAAGUAACAU | 856 | AUGUUACUUUGAGAGCCUUCUGC | 880 |
| CAUGAGCAUCAAAGAUCGGCA | 857 | UGCCGAUCUUUGAUGCUCAUGCU | 881 |
| AAGCAAUACAUUCCUUGAUGC | 858 | GCAUCAAGGAAUGUAUUGCUUUU | 882 |
| UUAAAACAAUUUCAAGCUCAC | 859 | GUGAGCUUGAAAUUGUUUUAAGU | 883 |
| GAGCAUGAGCAUCAAAGAUCG | 860 | CGAUCUUUGAUGCUCAUGCUCAU | 884 |
| UGUGUACAAACUCCCAAUUGC | 861 | GCAAUUGGGAGUUUGUACACAGC | 885 |
| AGUCAGGCUCCUCCAUAGCAG | 862 | CUGCUAUGGAGGAGCCUGACUCA | 886 |
| GGUGUCUGUUUCUUAGGAGUG | 863 | CACUCCUAAGAAACAGACACCAA | 887 |

In some embodiments, an oligonucleotide is a modified oligonucleotide as provided in Table 34, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 34
Modified Oligonucleotides for targeting MED1

| | Modified Passenger | | Modified Guide Strand/ | |
|---|---|---|---|---|
| | Strand/Sense | SEQ | Antisense | SEQ |
| | Strand (RNA) | ID | Strand (RNA) | ID |
| siRNA # | (5′ to 3′) | NO: | (5′ to 3′) | NO: |
| hsMED1-1 | mAmUfGmUfUmAfCmAfU | 840 | fAfUmAfUmCfUmGfAmCfGmUfG | 864 |
| | mCfAmCfGmUfCmAfGmAf | | mAfUmGfUmAfAmCfAmU*fU*mC | |
| | UmAfU | | | |
| hsMED1-2 | mAmCfAmUfCmAfCmGfU | 841 | fAfGmAfAmCfAmUfAmUfCmUfG | 865 |
| | mCfAmGfAmUfAmUfGmU | | mAfCmGfUmGfAmUfGmU*fA*mA | |
| | fUmCfU | | | |
| hsMED1-3 | mGmUfCmAfGmAfUmAfU | 842 | fUfCmCfAmCfAmUfAmGfAmAfC | 866 |
| | mGfUmUfCmUfAmUfGmU | | mAfUmAfUmCfUmGfAmC*fG*mU | |
| | fGmGfA | | | |
| hsMED1-4 | mUmGfAmUfCmAfGmAfC | 843 | fAfGmGfCmCfAmCfUmUfGmGfU | 867 |
| | mAfCmCfAmAfGmUfGmGf | | mGfUmCfUmGfAmUfCmA*fG*mA | |
| | CmCfU | | | |
| hsMED1-5 | mUmGfAmCfUmUfCmAfU | 844 | fAfCmAfAmCfUmUfUmGfGmCfA | 868 |
| | mUfGmCfCmAfAmAfGmUf | | mAfUmGfAmAfGmUfCmA*fU*mC | |
| | UmGfU | | | |
| hsMED1-6 | mCmAfAmAfGmUfUmGfU | 845 | fAfUmAfCmAfUmCfUmUfUmGfA | 869 |
| | mUfCmAfAmAfGmAfUmG | | mAfCmAfAmCfUmUfUmG*fG*mC | |
| | fUmAfU | | | |
| hsMED1-7 | mUmUfGmUfUmCfAmAfA | 846 | fUfGmGfAmCfAmUfAmCfAmUfC | 870 |
| | mGfAmUfGmUfAmUfGmU | | mUfUmUfGmAfAmCfAmA*fC*mU | |
| | fCmCfA | | | |
| hsMED1-8 | mGmCfCmCfAmGfGmGfU | 847 | fUfGmGfUmCfAmUfGmCfCmAfU | 871 |
| | mAfUmGfGmCfAmUfGmA | | mAfCmCfCmUfGmGfGmC*fU*mG | |
| | fCmCfA | | | |
| hsMED1-9 | mAmCfCmUfUmGfUmUfU | 848 | fCfAmUfGmCfUmCfAmUfAmUfU | 872 |
| | mAfAmUfAmUfGmAfGmC | | mAfAmAfCmAfAmGfGmU*fG*mG | |
| | fAmUfG | | | |
| hsMED1-10 | mCmCfAmCfCmCfGmAfU | 849 | fAfGmGfUmUfCmAfUmGfAmGfC | 873 |
| | mGfCmUfCmAfUmGfAmAf | | mAfUmCfGmGfGmUfGmG*fU*mU | |
| | CmCfU | | | |
| hsMED1-11 | mUmGfGmAfGmUfAmGfA | 850 | fUfCmAfGmGfAmUfUmGfAmAfA | 874 |
| | mUfUmUfCmAfAmUfCmCf | | mUfCmUfAmCfUmCfCmA*fU*mC | |
| | UmGfA | | | |
| hsMED1-12 | mCmCfAmGfCmAfGmUfG | 851 | fAfAmGfAmCfAmCfAmGfAmAfC | 875 |
| | mGfUmUfCmUfGmUfGmU | | mCfAmCfUmGfCmUfGmG*fU*mA | |
| | fCmUfU | | | |
| hsMED1-13 | mAmCfCmCfAmAfUmUfC | 852 | fAfCmAfAmAfCmUfGmGfUmAfA | 876 |
| | mUfUmAfCmCfAmGfUmUf | | mGfAmAfUmUfGmGfGmU*fU*mC | |
| | UmGfU | | | |
| hsMED1-14 | mGmGfGmAfGmGfAmCfU | 853 | fAfCmAfCmCfUmUfGmCfUmGfA | 877 |
| | mUfCmAfGmCfAmAfGmGf | | mAfGmUfCmCfUmCfCmC*fC*mA | |
| | UmGfU | | | |
| hsMED1-15 | mGmUfCmAfGmGfCmUfC | 854 | fUfCmUfGmCfUmAfUmGfGmAfG | 878 |
| | mCfUmCfCmAfUmAfGmCf | | mGfAmGfCmCfUmGfAmC*fU*mC | |
| | AmGfA | | | |
| hsMED1-16 | mUmGfGmUfGmUfCmUfG | 855 | fAfCmUfCmCfUmAfAmGfAmAfA | 879 |
| | mUfUmUfCmUfUmAfGmG | | mCfAmGfAmCfAmCfCmA*fA*mA | |
| | fAmGfU | | | |
| hsMED1-17 | mAmGfAmAfGmGfCmUfC | 856 | fAfUmGfUmUfAmCfUmUfUmGfA | 880 |
| | mUfCmAfAmAfGmUfAmA | | mGfAmGfCmCfUmUfCmU*fG*mC | |
| | fCmAfU | | | |
| hsMED1-18 | mCmAfUmGfAmGfCmAfU | 857 | fUfGmCfCmGfAmUfCmUfUmUfG | 881 |
| | mCfAmAfAmGfAmUfCmGf | | mAfUmGfCmUfCmAfUmG*fC*mU | |
| | GmCfA | | | |
| hsMED1-19 | mAmAfGmCfAmAfUmAfC | 858 | fGfCmAfUmCfAmAfGmGfAmAfU | 882 |
| | mAfUmUfCmCfUmUfGmAf | | mGfUmAfUmUfGmCfUmU*fU*mU | |
| | UmGfC | | | |
| hsMED1-20 | mUmUfAmAfAmAfCmAfA | 859 | fGfUmGfAmGfCmUfUmGfAmAfA | 883 |
| | mUfUmUfCmAfAmGfCmUf | | mUfUmGfUmUfUmUfAmA*fG*m | |
| | CmAfC | | U | |
| hsMED1-21 | mGmAfGmCfAmUfGmAfG | 860 | fCfGmAfUmCfUmUfUmGfAmUfG | 884 |
| | mCfAmUfCmAfAmAfGmAf | | mCfUmCfAmUfGmCfUmC*fA*mU | |
| | UmCfG | | | |
| hsMED1-22 | mUmGfUmGfUmAfCmAfA | 861 | fGfCmAfAmUfUmGfGmGfAmGfU | 885 |
| | mAfCmUfCmCfCmAfAmUf | | mUfUmGfUmAfCmAfCmA*fG*mC | |
| | UmGfC | | | |
| hsMED1-23 | mAmGfUmCfAmGfGmCfU | 862 | fCfUmGfCmUfAmUfGmGfAmGfG | 886 |
| | mCfCmUfCmCfAmUfAmGf | | mAfGmCfCmUfGmAfCmU*fC*mA | |
| | CmAfG | | | |
| hsMED1-24 | mGmGfUmGfUmCfUmGfU | 863 | fCfAmCfUmCfCmUfAmAfGmAfA | 887 |
| | mUfUmCfUmUfAmGfGmA | | mAfCmAfGmAfCmAfCmC*fA*mA | |
| | fGmUfG | | | |

k. MED13 Oligonucleotides

Examples of oligonucleotides useful for targeting MED13 are provided in Xu, M. et al. “MicroRNA-499-5p regulates skeletal myofiber specification via NFATc1/MEF2C pathway and Thrap1/MEF2C axis” Life Sci. 2018, 215:236-245.; and Grueter, C. E., et al. “A cardiac microRNA governs systemic energy homeostasis by regulation of MED13” Cell. 2012, 149(3):671-83.; the contents of each of which are incorporated herein in their entireties.

In some embodiments, oligonucleotides may have a region of complementarity to a human MED13 sequence, for example, as provided below by Gene ID: 9969; NCBI Ref. No: NM_005121.3:

(SEQ ID NO: 888)
CTCTCTCTGGTCGGAGGCGGCGGTAATGGCGGATGGTGGGTTGTGG
CGCCGGCGGCGGCTGCTGTGAGGGACGATGAGTGCCTCCTTCGTG
CCGAACGGGGCCAGCCTGGAAGATTGTCACTGTAACCTCTTCTGC
CTGGCTGACTTGACAGGAATTAAGTGGAAAAAATATGTATGGCAA
GGCCCAACTTCTGCCCCTATTCTGTTTCCTGTGACAGAAGAAGAC
CCCATTTTGAGCAGTTTTAGTCGCTGCCTTAAGGCAGATGTACTT
GGTGTTTGGCGGCGAGATCAAAGACCTGGAAGAAGAGAATTGTGG
ATATTTTGGTGGGGTGAAGACCCCAGTTTTGCTGACCTTATTCAC
CATGACTTATCAGAAGAAGAAGATGGAGTGTGGGAGAATGGACTT
TCCTATGAATGCCGTACTCTGCTTTTCAAAGCAGTTCACAATCTA
TTGGAACGGTGTTTAATGAACAGGAATTTTGTACGTATTGGCAAG
TGGTTTGTAAAGCCTTATGAAAAAGATGAAAAACCTATAAATAAA
AGTGAACACTTGTCCTGCTCCTTCACCTTTTTCTTGCATGGAGAC
AGCAATGTTTGTACCAGTGTGGAAATTAACCAACATCAACCTGTA
TACCTTCTCAGTGAAGAGCATATCACCCTTGCTCAACAGTCTAAT
AGCCCATTTCAAGTTATCTTATGCCCATTTGGACTAAATGGCACT
CTCACAGGACAGGCATTCAAGATGTCTGATTCAGCTACAAAAAAA
TTAATTGGTGAATGGAAACAGTTCTATCCTATCTCATGTTGCTTG
AAGGAGATGTCTGAAGAAAAACAGGAAGATATGGATTGGGAAGAT
GATTCTTTAGCTGCAGTAGAAGTTCTTGTTGCTGGTGTCCGAATG
ATCTACCCAGCATGCTTTGTTCTAGTCCCTCAGTCAGACATTCCT
ACTCCTAGCCCTGTGGGATCCACTCACTGTTCATCTTCTTGCTTG
GGTGTCCACCAAGTGCCTGCTTCCACAAGAGATCCTGCTATGTCT
TCGGTTACGCTTACACCACCTACGTCTCCTGAGGAAGTCCAAACA
GTTGATCCTCAGTCTGTCCAGAAGTGGGTCAAATTTTCTTCAGTA
TCTGATGGCTTCAACTCCGATAGTACTAGCCACCATGGTGGGAAA
ATACCCAGAAAATTAGCAAATCATGTGGTGGATAGAGTTTGGCAA
GAATGCAATATGAACAGAGCACAGAACAAGAGGAAGTATTCTGCT
TCATCAGGTGGTCTATGCGAAGAAGCGACAGCTGCTAAAGTGGCA
TCCTGGGATTTTGTTGAAGCCACACAAAGAACAAATTGCAGTTGT
TTGAGGCACAAAAATCTCAAGTCAAGAAATGCTGGACAACAAGGA
CAGGCACCATCTTTAGGTCAGCAACAACAAATACTTCCTAAGCAC
AAGACCAATGAGAAGCAAGAAAAGAGTGAAAAGCCACAGAAACGC
CCCTTGACTCCTTTTCACCATCGTGTGTCTGTTAGTGATGATGTT
GGCATGGACGCAGATTCAGCCAGCCAAAGACTTGTGATCTCTGCT
CCAGACAGTCAAGTGAGATTTTCAAATATCCGAACTAATGATGTA
GCAAAGACTCCTCAGATGCATGGCACCGAAATGGCAAATTCACCT
CAACCACCCCCACTTAGTCCTCACCCTTGTGATGTGGTTGATGAA
GGAGTGACTAAAACACCTTCAACTCCTCAGAGTCAACATTTTTAT
CAAATGCCAACACCAGATCCCTTGGTTCCTTCTAAACCAATGGAA
GATAGGATAGACAGTTTGTCCCAGTCTTTCCCACCTCAATATCAG
GAAGCTGTAGAACCTACAGTATATGTTGGTACAGCAGTAAACTTG
GAAGAAGATGAAGCCAATATAGCCTGGAAGTATTACAAGTTCCCA
AAGAAAAAAGATGTAGAGTTTTTACCACCTCAACTTCCAAGTGAT
AAATTCAAGGATGATCCAGTTGGACCTTTTGGACAGGAAAGTGTA
ACATCAGTTACAGAGTTAATGGTGCAATGTAAGAAACCTTTAAAA
GTTTCTGATGAATTAGTGCAGCAATATCAAATTAAAAACCAGTGT
CTTTCAGCAATAGCATCTGATGCAGAACAAGAACCTAAAATTGAT
CCATATGCATTTGTTGAAGGAGATGAGGAATTCCTTTTTCCTGAT
AAAAAAGATAGACAAAATAGTGAGAGAGAAGCTGGAAAAAAACAC
AAGGTAGAAGATGGGACATCTAGTGTAACAGTGTTATCACATGAA
GAAGATGCTATGTCATTATTTAGTCCCTCTATCAAGCAAGATGCT
CCACGCCCTACTAGTCATGCCCGTCCTCCATCAACAAGTTTGATT
TATGACTCAGACCTGGCTGTCTCTTATACTGACCTTGATAATCTC
TTCAATTCTGATGAAGATGAACTAACACCTGGATCTAAAAAATCA
GCAAATGGATCAGATGATAAAGCCAGCTGCAAGGAATCAAAGACA
GGAAATCTGGACCCGTTATCTTGCATAAGCACTGCAGATCTTCAT
AAAATGTATCCTACACCACCATCATTGGAACAACATATTATGGGA
TTTTCCCCAATGAATATGAATAATAAAGAATATGGTAGTATGGAT
ACAACACCTGGAGGAACTGTTCTAGAAGGAAATAGTTCTAGTATA
GGAGCGCAGTTCAAAATTGAGGTTGATGAGGGATTCTGTAGCCCC
AAACCTTCTGAAATTAAAGATTTTTCTTATGTCTATAAGCCTGAA
AATTGTCAAATTCTAGTGGGATGTTCCATGTTTGCACCTCTAAAA
ACTCTACCAAGCCAATATCTGCCCCCTATCAAATTGCCAGAAGAG
TGTATTTACCGTCAGAGTTGGACTGTTGGAAAATTGGAATTGCTT
TCTTCAGGGCCTTCAATGCCATTCATCAAAGAGGGTGATGGAAGT
AATATGGATCAAGAATATGGCACTGCTTATACACCTCAAACTCAT
ACTTCTTTTGGGATGCCTCCTAGCAGTGCACCTCCTAGTAACAGC
GGAGCAGGAATTCTTCCTTCTCCATCCACCCCTCGGTTTCCAACT
CCAAGGACTCCAAGGACTCCTCGGACTCCTCGTGGAGCTGGTGGA
CCTGCTAGTGCTCAAGGTTCAGTCAAATATGAAAATTCAGACTTG
TATTCACCAGCTTCTACCCCATCTACATGCAGACCCCTTAATTCT
GTTGAACCTGCAACTGTCCCTTCCATCCCTGAAGCACACAGTCTT
TATGTAAACCTCATCCTTTCAGAATCAGTTATGAATTTGTTTAAA
GACTGTAACTTTGATAGTTGTTGCATCTGTGTTTGCAACATGAAC
ATCAAGGGTGCCGATGTTGGAGTTTACATTCCAGATCCAACGCAG
GAAGCACAATATAGGTGTACCTGTGGCTTCAGTGCTGTCATGAAC
AGAAAATTTGGAAACAATTCAGGATTATTTCTTGAAGATGAACTA
GATATCATAGGACGCAATACAGACTGTGGCAAAGAAGCAGAAAAA
CGTTTTGAAGCTCTCAGGGCTACCTCTGCTGAACATGTTAATGGA
GGACTAAAGGAATCTGAAAAATTATCTGATGATTTGATATTATTG
CTACAAGATCAGTGCACTAATTTATTTTCACCCTTTGGAGCAGCA
GACCAAGATCCTTTTCCTAAAAGTGGTGTAATTAGCAATTGGGTA
CGTGTTGAAGAGCGTGACTGTTGCAATGACTGCTACCTTGCATTA
GAACATGGGCGTCAGTTCATGGATAACATGTCAGGAGGAAAAGTT
GATGAAGCACTTGTGAAAAGTTCATGCTTACACCCCTGGTCCAAA
AGAAACGATGTGAGTATGCAGTGCTCACAGGATATACTTCGAATG
CTCCTCTCTCTTCAGCCAGTTCTTCAGGATGCCATTCAGAAAAAA
AGAACAGTAAGACCTTGGGGTGTTCAGGGTCCTCTCACTTGGCAA
CAATTTCATAAAATGGCTGGCCGAGGCTCTTATGGAACTGATGAA
TCCCCAGAACCACTGCCAATCCCCACATTTTTGTTGGGTTATGAT
TATGATTATCTGGTGCTTTCTCCATTTGCTCTTCCTTATTGGGAG
AGACTTATGCTGGAACCCTATGGATCTCAAAGAGATATAGCCTAT
GTTGTACTGTGTCCAGAAAATGAAGCCTTGTTAAATGGAGCAAAA
AGCTTTTTTAGAGATCTTACTGCAATATATGAGTCCTGTCGATTA
GGTCAACATAGACCTGTTTCTCGACTGTTAACAGATGGGATCATG
AGAGTTGGATCTACTGCATCAAAGAAACTATCAGAAAAGTTGGTA
GCAGAATGGTTTTCTCAGGCAGCTGACGGTAACAATGAAGCATTT
TCTAAACTCAAGCTTTATGCACAAGTCTGCAGATATGACCTAGGT
CCTTATCTTGCTTCCCTGCCATTGGACAGCTCTCTACTTTCCCAG
CCAAATTTAGTTGCCCCTACAAGTCAGTCTTTGATTACTCCACCT
CAGATGACAAATACTGGAAATGCTAATACTCCATCTGCCACCTTA
GCATCTGCAGCGAGCAGCACTATGACAGTGACTTCAGGTGTTGCC
ATATCTACTTCAGTTGCCACAGCTAATTCAACTTTGACCACAGCT
TCAACTTCATCTTCATCATCCTCCAACTTGAATAGTGGAGTATCA
TCAAATAAACTACCTTCGTTTCCACCCTTTGGCAGTATGAACAGT
AATGCTGCAGGATCCATGTCTACACAAGCAAATACAGTTCAGAGT
GGTCAGCTAGGAGGGCAACAGACATCAGCTCTACAGACAGCTGGG
ATTTCTGGAGAATCATCTTCACTTCCCACTCAGCCGCATCCTGAT
GTGTCTGAAAGCACGATGGATCGGGATAAAGTGGGAATCCCCACA
GATGGTGATTCACATGCAGTCACGTATCCACCTGCAATTGTTGTT
TATATAATTGATCCTTTTACATACGAAAATACAGACGAGAGCACT
AACTCTTCTAGTGTGTGGACATTGGGGCTACTTCGATGCTTTCTA
GAAATGGTCCAGACTCTTCCTCCTCATATCAAGAGTACTGTTTCT
GTACAGATTATTCCTTGTCAGTACCTGTTGCAACCTGTGAAGCAT
GAAGATAGAGAAATCTATCCCCAGCATTTAAAATCCCTGGCTTTT
TCGGCCTTTACCCAGTGTCGGAGGCCACTTCCAACATCAACCAAT
GTGAAAACATTGACTGGCTTTGGTCCAGGTTTAGCCATGGAAACT
GCCCTTAGAAGTCCTGATAGACCAGAGTGTATTCGACTTTATGCA
CCTCCTTTTATTCTGGCTCCAGTGAAGGACAAACAGACAGAGCTA
GGAGAAACATTTGGAGAAGCTGGACAGAAATATAATGTTCTTTTT
GTGGGATACTGTTTATCACATGATCAAAGGTGGATTCTTGCATCT
TGCACAGATCTATATGGAGAACTTTTAGAAACTTGTATCATTAAC
ATCGATGTTCCAAATAGGGCTCGTCGGAAAAAAAGTTCTGCTAGA
AAATTTGGTCTACAGAAACTTTGGGAGTGGTGCTTAGGACTTGTA
CAAATGAGTTCATTGCCATGGAGAGTTGTAATTGGTCGTCTAGGA
AGGATTGGTCATGGAGAATTGAAAGATTGGAGCTGTTTGCTGAGT
CGTCGAAACTTGCAGTCTCTAAGTAAAAGGCTCAAAGACATGTGT
AGAATGTGTGGTATATCTGCTGCAGACTCCCCTAGCATTCTCAGT
GCTTGCTTGGTGGCAATGGAGCCGCAAGGCTCTTTTGTTATTATG
CCAGATTCTGTGTCAACTGGTTCTGTATTTGGAAGAAGCACGACT
CTAAATATGCAGACATCTCAGCTAAATACCCCACAGGATACATCA
TGTACTCATATACTTGTGTTTCCTACTTCTGCTTCTGTGCAAGTA
GCTTCAGCTACTTATACCACTGAAAATTTGGATTTAGCTTTCAAT
CCCAACAATGATGGAGCAGATGGAATGGGTATCTTTGATTTGTTA
GACACAGGAGATGATCTTGACCCTGATATCATTAATATCCTTCCT
GCTTCTCCAACTGGTTCTCCTGTACATTCTCCAGGATCTCATTAC
CCCCATGGAGGTGATGCGGGCAAGGGTCAGAGTACTGATCGGCTA
CTATCAACAGAACCTCATGAGGAAGTACCTAATATTCTTCAGCAA
CCATTGGCCCTTGGTTACTTTGTATCAACTGCCAAAGCAGGTCCA
TTACCTGACTGGTTCTGGTCAGCATGTCCTCAAGCACAATATCAG
TGTCCCCTTTTTCTTAAGGCCTCTTTGCACCTCCACGTGCCTTCA
GTGCAATCTGACGAGCTGCTTCACAGTAAACACTCCCACCCACTT
GACTCAAATCAGACTTCAGATGTCCTCAGGTTTGTTTTGGAACAG
TACAATGCACTCTCCTGGCTAACCTGTGACCCTGCAACCCAGGAC
AGACGCTCATGTCTCCCAATTCATTTTGTGGTGCTGAATCAGTTA
TATAACTTTATTATGAATATGCTGTGATCTTCATTTGATGGAACT
GTGCAAGAAAAGAACAAGGAAAAATGGATGTTTCGCTGCAGGATT
AAGTTACAATTATCTTCTCAGTGAAGGTCATTTGTGATGGGGTCT
AATTCTTATTACTTCAACAAATATTGTTTTGACTTGGGGGGAGGG
GCTATAACCCTGCTATTTTTCATTGACTCTATTGAACTCTTTAGG
ATGATGACTGATCATACAAAACGTATTATAACATTTTCGTAGCAA
AATTAACCTTTTTTTTTTCCAGTCACAGTATTTGTGAAAAGTAAT
GAGCCATAGTACCCAGTCATGTTAAATGAATATTAAAAGCATGGA
GAGGAAACATGAGGAACAATGAATTTCAACATATGGCTTCAGAAC
ATGAAGATGTTCTTGTATGGATTATAGTATCTAGTATTCAAAAAT
GCCTGCATCTCTTCTCTTATTTATTGTAAGTTTTTAAATGTATAA
ATTGTCTTATATTTCTTAACCTCTTTTATAAAAATTTTCCTAGAA
GGTTTATACTGCCTTCTTGCTTTAAAGCAATTGGTCTAAAATATA
TGTAATCGTCTTAATTAAAAAGTTGCAGTAGGGTTGCTTTTAGAG
TATTATTTTTTTGTAAGGGGGTGGGTGGGACAGTAAATTTGTATT
GTCTCGATGTACAGTTTAACGGGGATAGAGGGGGAATAATGTCCA
TACCATTGTGTGTGGAGGATTTACAGCTAAGCTGTAGTTGCAGAG
TACATGTACAGTAATGAAGTTCACTGTGTTTATAAATTGAAAAGG
TACCAGGTCTTACAGCATTTTATATATCACATCTTTACAGAATAA
CATGATGGCAATATACAAGTGGTATTGTTAGGTGGTTTAACTTAG
AATAAAATGAGAATTCTTCAGTTATATTTTGTACTATGGTTTAGG
GCTATGACTAATATTTCAGGCCATTTCCGGTGAAAGAAACTTAGT
TTTACAAGAAAAACCATTTGCTACTGAATGCTTAAACTAATTTTA
GTGTTTAATGTTACATGCTTAAATTTTTTTCAGTTTTAACAGTGG
CATATTTAGGCATGGAAATATTATTATGAAATTTATTTTCAGGAT
CTGCTATAAGGTTGAAATTTAGCCCAGCTCTAGGCATTTTACAAA
TTATTTTTCAAGCAGTCATTCTTGATTGTTTGACTTTTTTTTTTA
AATTAAAGATTGGGAATGTATGTGAGAGTATGCATATGTATGGGT
GTGTGTGTGTGCGCGCAATCAAACTGTGGTGTAAATAGATTCTCA
GTGAATTCTGGTATTCAGACTCTATTCCACTAGTGAAAGAACCAT
TTTTTAAACTTCCCTTGCCTTTTTTATTTATTTAATTTTCTTGGT
TTGGAGATGTCAGTCCCAAACACCAGAGTCTGTACTTTTCTATAA
CACAGCTCAGATTAAGGTAGGGCATATGCCAAGGAGGTTCTCACC
TCCCTAAAGAAGGGACTTGAATTTTAGGGACTTTAATTCACCCCT
CCTTCAATACAACTTTCCCCCTTCTTGTTTGCACATGCCAAGATA
ACTGCTTTTATGCAGGCTGTACCCCCTTGAAAAATCCTTTCTACA
GTGCTGCTCACAAAAGAGCCCAAGTTCGCCTCCTACCTGCATTGC
TGACTTGAATTCACAGTCGCCGAGTCTACCTAGCTTTCTTGGAAG
CAGTCTAGCAAAATTTCTATTTGTACGTTCACTAATTATCTACAA
GGACAAAATCAGTTGTATTTACAAAACTCTACTTCAGTGTTTGTT
TTAGTTTTTTTTTTTACTGAAACTTGTTTTTGTGAATACTCTGTG
CTTAGAATTAAATATCACTTTCTTATGAACAACATAACTTCTTCA
GATTGTGTATATGAAAACATTAGCAAGTCTTGTTTTTTCTATGAA
GCAAACACAATTGGTGACAAAGGTTGTCAATCATTTCTTCAAAAT
TATAATGCAGTTCTAATGGTCAGCATATTTTGATATTAAATTTAA
AGATCACCTCTCTGCATTTGTTTTTAAATTATGCTAATACACCAC
ACATTATGTTGGTATGTTTTGTTCTGTACTTTCTTTAAAAAAAAA
AAAAAAACTTGTCTGAGATTTGAAGGAAAATGTGCTTATTTGGAA
TTTCCATAAAAAGAGTATCCTTTTTATACACTTAATAGTGACTTT
ACAAAATAAAAGTTATATTCTCAGTTGTTTAAAATCACTAACCTA
TGATAACCACACCTCAATTTGAAAGTAGATTTAAAATTATTCCCT
GACAGGTTATTTAATATGGAGCCATAAGGAGGGAACCCAGTACAC
AATTATTTTTTATTTGGGAATCAGGGAATAGTTCCCAAATATACA
GGATTTATTGATAAGATTTTTTTTCTTCCCTTCATATATCCATTC
AAACTCAATGGAAAGTTATTAAATAACCATTAGAAAAGCTCAGTA
GACTTATTTGAGAAATTAAGCCTTGTGCAGGATGATGGATTTGAC
TTACTAATGTACTGTCACAGACAAATATGGGTAGTTTTGTTTAAA
TAGGTAAGCAAAATATTATACTTTATAGCAGTGGATTACCAACAC
CTTGACTTCTTTGTTACAGTGCTAACATCTTTTTTTTTGTGCAGG
TATCCATGATTATTAAGCAGGGTGGAAGTTCAGTATTTTGTCATT
TAAAAAGATTAGTTATATAATGTCTGCTTCCAGCCAGTGAGAAAC
ATCTAGCCATACCTTTCTTATGCAAGCCATTGAGTTATCAGGACT
GTGAATTAACACTGTATGAATAAATTTCTGTACACCTTATTGTTT
GGCCAGAAGGCCACCAAGTGTACTTATATGTAATCCTTAAATTTT
AAAGTAGCTGTAATTTTTAAATATTTCTAAACTTTTCTTAAACCA
CTAAAATTAAGCTCTTACTACTTAGTCAACTATCCTCAGCTGTAT
TCGTACTCAATTGTCAGTATGGCACAGATTACTGTATTAAAATAT
TCTCCTTTCGTCTTCATATTTACCTTCTGAGGTAATTTTTTAACT
TAATGTGTTACTACAAAGATTTGCAGATCTTTAATCAAGCACTAT
GTTAATACTGTAATATCAGAATACTATGTTGCATTATTTAAAATG
TTCAAATTGAATAGATTAAAAAGTTTTTAAATGCTATTGCATCAT
ATAATTTGCTATTCATCCACTATGGCATTGCATATCAATCAGTTA
ATACACTTAATGTTGCATGAGTGATATTTTGGTCTGGGTTTCCTC
TTAAGATTTTAGTTTGTCTGAATTAAGGAAAAATGTTTTTAATAT
ACATTCTTATTTTGTCCCACCCCTCCAGAAATAAGCTGGAAATCT
TAACTTTTTGGGGGGTCTTTTTTGGTGTTTTAATGGGCCCAGAAC
TGTGGTTTAAATTTTTATGTATGTATTTTCTTTTTTGTGGAGTAT
AAATTTAAAAACTGGATTTGGGACCTAAAATACTCCTCAGGTTGA
TGTATTCATGAAGTTTTAAAACATCTTTAGTTTTCAAAGTAAACT
GGATATGTGGACCTTAAAGTTATTGAGTTTAAGCTACAAATTGTA
ACGTCATTACTGGACATGTCAGCATCAACCCTCTCAAAATAGCTT
GGTCACTTTATGAAGGGGCGTTTTAAAGTTGTTGTTTAGCAGTGA
CATTTAATATGGTCCAATTGCTTTTCTTTTTAACGTGACAAAAAG
AGAATAAGGAACAAACACTATTGCTGCCGAATGCCATAACACTGA
GTTGTACAAATTGTGATTGAGGAAATGAAAAGGTTTATACTTTTT
AAAAAAAAAAAAACAAAAACAAAAAACAAAACTTCAAATGGAATA
AATTATTCATGAAGCCTTCA

In some embodiments, oligonucleotides may have a region of complementarity to a mouse MED13 sequence, for example, as provided below by Gene ID:327987; NCBI Ref. No: NM_001080931.2:

(SEQ ID NO: 889)
CTCTCTGGTCGGAGGCGGCGGTAATGGCGGATGGTGGGTTGTGGCGCCG
GCGGCGGCTGCTGTGAGGGACGATGAGTTCCTCCTTCGTGTCGAACGGG
GCCAGCCTGGAAGATTGTCACTGTAACCTCTTCTGCCTGGCCGACTTGA
CAGGAATTAAGTGGAAAAGATATGTATGGCAAGGCCCAACTTCCGCCCC
TATTCTGTTTCCGGTGACAGAAGAAGACCCCATTTTGAGCAGTTTTAGT
CGATGCCTTAAGGCAGATGTACTTGGTGTTTGGCGGCGAGATCAAAGAC
CTGGAAGAAGAGAATTGTGGATATTTTGGTGGGGTAAAGATCCCAATTT
TGCTGACCTTATTCACCATGACTTATCAGAAGAGGAAGATGGAGTATGG
GAGAATGGCCTTTCCTATGAATGCCGTACTTTGCTTTTCAAAGCAGTTC
ACAATCTGTTGGAGCGATGTTTGATGAACCGAAACTTTGTACGAATTGG
CAAGTGGTTTGTGAAGCCATATGAGAAAGATGAAAAACCTATAAATAAA
AGTGAACACTTGTCCTGCTCTTTCACCTTTTTCTTGCATGGAGACAGCA
ATGTTTGTACCAGTGTGGAAATTAACCAACATCAACCTGTATACCTTCT
CAGTGAAGAGCATGTCACCCTTGCTCAACAGTCTAATAGCCCATTTCAA
GTTATCTTAAGTCCATTTGGACTAAATGGCACTCTCACGGGACAGGCTT
TCAAGATGTCTGATTCAGCTACGAAAAAATTAATTGGTGAATGGAAACA
GTTCTATCCTATTTCATGTGGTTTGAAGGAGATGTCTGAGGAGAAACAG
GACGATATGGATTGGGAAGACGATTCTTTGGCTGCGGTGGAAGTTCTTG
TTGCGGGTGTCCGAATGATCTACCCAGCATGCTTTGTGCTAGTCCCTCA
GTCAGACATTCCTGCTCCTAGCTCTGTGGGAGCTTCTCACTGTTCAGCT
TCTTGCTTGGGTATCCATCAAGTGCCTGCTTCCACAAGAGATCCTGCTA
TGTCTTCAGTTACTCTCACTCCACCTACATCTCCTGAGGAAGTCCAAAC
AGTTGATCCTCAGTCCGCACAGAAGTGGGTCAAATTTTCTTCAGTATCC
GATGGCTTCAGCACTGACAGTACTAGCCATCATGGTGGGAAAATACCCA
GAAAATTAGCAAATCATGTGGTGGATAGAGTCTGGCAAGAATGTAATAT
GAACAGATTACAGAACAAGAGGAAGTATTCTGCTACATCCAGCGGCCTG
TGTGAGGAAGAGACTGCCGATAAAATAGGATGCTGGGACTTTGTTGAAG
CCACACAAAGAACAAGCTGCAGTTGTTTGAGGCATAAAAGTCTCAAGAC
AAGAAATACTGGGCAACAAGGACAGGCACCATCTTTAGGGCAGCAACAG
CAAGTACTTCCTAAGCACAAGACCAATGAGAAGCAAGACAAGAGTGAGA
AGCCACAGAAGCGCCCCTTGACTCCCTTTCACCATCGTGTATCAGTTAG
TGATGAGATTGGCATGGACACAGATTCAGCCAGCCAAAGACTTGTGATC
TCTGCTGCAGATAGTCAAGTGAGGTTTTCAAACATCCGAACTAATGATG
TAGCAAAGACTCCTCAGATGCATGGCACTGAACTGGCAAATTCACCTCA
GCCTCCCCCACTTAGTCCTCATCCTTGTGATGTGGTTGATGAAGGAGTG
ACTAAAACACCTTCAACTCCGCAGAGTCAACATTTTTATCAAATGCCAA
CACCGGATCCCTTGGTTCCTACCAAACCAATGGAAGATAGGATAGACAG
TTTGTCCCAGTCTTTCCCACCTCCATTTCAGGAAGCTGTAGAACCTACC
GTGTATGTTGGTACAGCAGTAAGCTTGGAAGAAGATGAAGCTAATGTAG
CCTGGAAGTATTACAAAGTCCCAAAGAAAAAAGATGTAGAATTTTTACC
ACCTCAACTTCCAAATGATAAATTCAAGGATGATCCAGTTGGACCTTTT
GGACAGGAAAGTGTAACATCAGTTACAGAGTTAATGGTGCAGTGTAAGA
AGCCTTTAAAAGTTTCTGATGAAATAGTCCAGCAATATCAAATTAAAAA
TCAGTATCTTTCAGCAATAGCATCTGATACAGAACAAGAACCTAAAATT
GATCCATATGCATTTGTTGAAGGAGATGAGGAATTCATTTTTACTGATA
AAAAAGATAGACAAAATAGCGAGAGAGAAGCTGGGAAGAAACACAAGGT
GGAAGATGGGACATCTGCTGTCACAGTGTTATCACACGAAGAAGATGCT
ATGTCATTATTTAGTCCCTCTAAACAAGATGCTCCACGCCCTACAAATC
ATGCCCGTCCTCCATCAACCAGTTTGATTTATGACTCAGATTTGGCTGT
GTCTTACACTGACCTTGATAACCTCTTCAATTCTGATGAAGATGAATTA
ACACCTGGATCTAAAAAGTCAGCAAGTGGATCAGATGATAAAGCCAGCT
CCAAAGAATCAAAGACAGGAAATCTGGATCCGTTGTCCTGCATAAGCAC
TGCAGACCTCCACAAAATGTACCCCACGCCACCCTCACTGGAACAGCAC
ATTATGGGCTTCTCTCCAATGAATATGAATAACAAAGAATACGGTAGTG
TGGACACAGCACCCGGAGGGACTGTCCTGGAAGGAAATAGCTCTAGTGT
AGGAACGCAGTTCAGAATCGAGGTCGAGGAAGGATTCTGTAGCCCTAAA
CCGTCTGAAATTAAAGATTTTTCTTATGTCTATAAGCCCGAAAATTGTC
AAGTTCTAGTAGGATGTTCCATGTTTGCACCTTTAAAAACTCTACCAAG
CCATTGTCTGCCTCCTATCAAATTGCCAGAAGAGTGTGTTTACCGGCAG
AGCTGGACTGTTGGAAAATTGGACTTGCTTCCTTCAGGCCCTTCAATGC
CATTCATCAAGGAGGGTGATGGCAGTAATTTGGATCAGGACTATGGCCC
CGCCTACACACCGCAAACCCATGCTTCTTTTGGGATGCCTCCGAGCAGC
GCACCTCCTAGTAATGGTGGTGCAGGAATTCTTCCTTCCCCATCCACCC
CTAGGTTTCCAACTCCAAGGACTCCAAGGACTCCAAGGACCCCTCGTGG
GGCTGGTGGGCCTGCTAGTGCTCAAGGTTCAGTCAAGTATGAGAATTCA
GACTTGTATTCACCAGCTTCCACCCCATCTACATGCCGACCCCTCAATT
CTGTTGAACCTGCAACTGTTCCTTCCATCCCTGAAGCGCACAGTCTTTA
TGTAAACCTCATCCTTTCAGAATCAGTAATGAATTTGTTTAAAGACTGT
AATTTTGATAGTTGCTGTATTTGTGTCTGCAATATGAATATCAAGGGTG
CCGATGTTGGAGTTTACATTCCAGACCCAACCCAAGAAGCACAGTATCG
GTGCACCTGTGGCTTCAGTGCTGTCATGAACCGAAAATTTGGAAATAAT
TCAGGATTATTTCTTGAAGATGAACTTGACATCATAGGACGTAACACAG
ACTGTGGGAAAGAAGCAGAAAAACGTTTTGAAGCTCTCAGGGCTTCCTC
TGTGGAGAATGTTAATGGAGGCCTGAAGGAATCTGAAAAGGTGCCTGAT
GAATTAATATTACTGCTACAAGATCAATGCACTAATTTATTTTCACCCT
TTGGAGCAGCAGACCAAGATCCTTTTCCCAAAGTGGGTATAAGTAGCAA
TTGGGTGCGTGTTGAGGAGCGGGACTGTTGTAGTGACTGCTGCCTCGCC
TTAGAACATGGGCGTCAGTTCATGGATAATATGTCAGGAGGGAAAGTTG
ATGAAGCACTTGTCAGAAGCTCATGCTTACATCCGTGGGCCAAGCAAAA
TGATGCGAGCGTGCAGTGTTCACAGGATATACTTCGGATGCTCCTTTCT
CTTCAGCCAGTTCTTCAAGATGCTATTCAGAAAAAAAGAACAGTGAGGC
CTTGGGGTGTTCAAGGTCCTCTCACATGGCAACAATTTCATAAAATGGC
TGGCCGAGGCTCTTATGGAACTGATGAATCTCCGGAACCACTGCCGATT
CCCACGTTTTTGTTGGGTTATGATTACGATTTTCTGGTGCTTTCTCCAT
TTGCTCTCCCTTACTGGGAGAAACTTATGCTGGAACCCTATGGATCTCA
AAGAGATATAGCCTATGTTGTACTCTGTCCAGAGAATGAAGCTTTGTTA
AATGGAGCCAGAAGCTTTTTTAGAGATCTCACTGCAATATATGAGTCCT
GTCGATTAGGCCAACACAGACCTATTTCTCGATTGTTGACGGATGGGAT
CATGAAAGTGGGAGCTACTGCATCAAAGAAACTTTCAGAAAAGTTTGTA
ACAGAATGGTTTTCTCAGGCAGCTGATGGTAACAATGAAGCATTTTCCA
AATTGAAGCTGTATGCACAAGTCTGCAGATATGATTTAGGTCCATATCT
TGCTTCTCAGCCGTTGGACAGCTCTCTGCTCTCCCAGCCAAATTTAGTT
GCCCCTCCAAATCAGTCTTTGGTTACTGCACCTCAGATGACAAATACTG
GAAACGCTAATGCTCCATCTGCCACCTTAGCATCTGCGGCAAGTAGCAC
TATGACAATGACTTCAGGTGTTCCCATATCTACTTCAGTTGCCACAGCT
AATTCAACTTTGACCACAACTTCATCTTCATCATCTTCCAGTCTGAGTA
GTGGAGTATCATCAAATAAACTGCCTTCGTTTCCACCCTTTGGCAGTAT
GAACACTAGTGGTACAGGATCCATGTCTGCACAAGCAAGTACGGTTCAG
AGTGGCCAGCTAGGAGGACAGCAGTCGTCATCTCTGCAGGCAGCAGGAA
TATCAGGGGAGTCAGCTTCTCTTCCCACTCAGCCACATCCTGATGTGTC
TGAAAGCACAATGGATCGTGATAAAGTGGGGATTCCCACAGATGGTGAT
TCACATGCAATTACCTACCCTCCTGCAATTGTTGTTTATATAATCGATC
CTTTTACATATGAAAATAAAGACGAGAGCACTAACTCTTCTAATGTATG
GACTTTGGGGCTCCTTCGATGCTTCTTGGAAATGGTCCAAACTCTTCCT
CCTCATATCAAGAGCACTGTTTCTGTGCAGATTGTTCCTTGTCAGTACC
TGTTACAACCTGTGAAACACGACGACAGACAGATCTACTCCCAGCATTT
AAAATCTTTGGCTTTTTCGGTGTTTACCCAGTGTCGGAGACCACTTCCA
ACATCAACCAATGTAAAAACACTGACTGGGTTTGGTCCAGGCTTAGCTA
TGGAAACTGCTCTTAAAAGTCCTGATAGACCAGAGTGTATTCGACTTTA
TACACCACCTTTTATTCTGGCTCCGGTGAAGGACAAACAAACAGAGCTA
GGAGAGACCTTTGGAGAAGCTGGACAGAAATACAATGTTCTTTTTGTGG
GCTACTGTTTATCACATGATCAAAGGTGGATTCTTGCATCTTGCACAGA
TCTATATGGAGAACTCCTAGAAACATGTATCATTAACATCGATGTTCCA
AATAGGGCCCGTCGGAAAAAGGGTTCTGCTAGAAGATTTGGTCTACAGA
AACTGTGGGAGTGGTGCTTGGGACTTGTTCAGATGAGCTCTTTACCATG
GAGAGTTGTGATTGGTCGCCTGGGAAGGATTGGACATGGAGAGTTGAAA
GATTGGAGTTGTTTGCTGAGTCGTCGAAACTTGCAATCTCTAAGCAAAA
GGCTCAAAGACATGTGTAGGATGTGTGGCATATCTGCTGCAGACTCTCC
AAGTATCCTTAGTGCTTGTTTGGTTGCAATGGAACCCCAAGGTTCTTTT
GTTATCATGCCAGATTCTGTGTCAACTGGTTCTGTTTTTGGAAGAAGTA
CCACTCTAAATATGCAGACGCCTCAGCTAAATACCCCACAAGATACATC
CTGTACTCATATACTTGTGTTCCCTACTTCTGCTTCTGTGCAAGTAGCT
TCAGCTACTTACACCACTGAAAATTTGGATTTAGCTTTCAATCCCAACA
ATGATGGAGCAGACGGAATGGGTATCTTTGATTTATTAGACACAGGAGA
TGATCTTGATCCTGATATCATTAATATCCTTCCTGCATCTCCAACTGCA
TCTCCTGTACATTCTCCAGGCTCTCATTACCCCCATGGAGGTGATGCTG
GCAAGGGTCAGGGTACTGATCGGCTACTTTCAACAGAATCGCATGATGA
AGTAACTAATATTCTTCAACAACCATTGGCCCTTGGATACTTTGTATCA
ACTGCCAAAGCGGGTCCATTACCTGACTGGTTCTGGTCAGCATGCCCTC
AAGCACAATATCAATGCCCCCTTTTTCTTAAGGCCTCTTTGCACCTCCA
CGTGCCTTCAGTGCAATCTGACGAGCTGCTTCACAGCAAACACTCCCAC
CCACTGGACTCAAATCAGACTTCAGATGTCCTCAGGTTTGTTTTGGAAC
AGTACAATGCACTCTCATGGCTAACCTGTGACCCTGCAGTCCAGGACAG
ACGCTCATGTCTCCCAGTTCATTTTGTGGTGCTGAATCAGTTATATAAC
TTCATCATGAATATGCTGTGATCCTCGTTTGATGGAATTACGCAAGGAA
AGAACGGGAAGAAAGGATATTTTGCTGCAAGATTAAGTTACAGTTCTCA
GTGGAAGTCATTTGTGATGGGGTCTAATTCTTATTACTTCAACAAATAT
TGTTTTGACTTGGGGGGCGGGGCTATAACCCTATTTTTCATTGACTTTA
CTGAGCTCTTTAGGATGATGACTGATTATTCAAAACGTATTATAACACT
TTCGTAGCAAAAATTAACCTTTTTTTTCCAGTCACAGTATTTGTGAAAA
GTAATGAGCCATAGTACCCATTAATGTTAAAGAATATTAAAGCATGGAG
AGGAACATGAGGAACAATGAATTCCAACATATGGCTTTAGAACATCAGG
ATGTTCTTGTATTGGATATAGTATCTAGTATTCAAAAATGCCTGCATCT
CTTATTTATTGTAAGTTTTTAAATGTATAAATTGTCTTATATTTCTTAA
CCTCTTTTATAAAAATTTTCCTAGAAGGTTTATACTGCCTTCTTGCTTT
AAAGCAATTGGTCTAAAATATATGTAATCGTCTTAATTAAAACGTTGCA
GTAGGGTTGCTTTTAGAGTATTATTTTTTTGTAAGGGGGTGGGGTGGAC
AGTAAATTTGTATTGTCTCAATGTACAGTTTAACAGGGATAGAGGGGAA
ATAATGTCCATGCCATTGTGTGTGGAAGATTTACAGCTAAGCTGTAGTT
GCAGAGTACATGTACAGTAATGAAGTTCACTGTGTTTATAAACTGAAAA
GGTACCGGGTCTTACAGCATTTTATATATCACACCTTTACAGAATAACA
TGATGGCAATATACATACGTGATATTGTTAGGTGGAATAACTTAGAATA
AAATGAGAATTCTTGAGTTATATTTTGTACTATGGTTTAGGGCTATGAC
TAATATTTCAGGCCATTTCCAGTGAAAGAAACTTAGTTTTACAAGAAAA
CATTTGCTACTGAATGCTTACACTAATCTTGGTGTTTAATGTTACATGC
TTAAATTCTTTTCAGTTTTCACAGTGGCACATTTAGGCATGGGGATAGT
ACTAATTATGAAATTTCCTGCAGGATCTGTTCTAAGGTTGAAATTTAGC
CCAGCTCTAGGCATTTTACAAATTATTTTAAGAACAGTCACTCTTGATT
GTTTTACTTTTTATTTTTAAATTAAAGATTGGGAATGCGTGTGAGAGTA
TGCATAAGCATGGCAGGAAGAGAGGGGGGAGAGAGAAAGAGAGAGAGAG
TGAGAAAGAAAGAGAGAGTGAGAGTGTGTGTGTATATGTGTGTGTGTGT
GCAATCAAACTGTGGTGTAAATAGATCCTCAGTGAATTCTGGTATTCAG
ACTCTGTTCCACTAGTGAATGAACCATTTTCTAAACGTTTCCCTTTGAC
TTTTTTATTTATTGAATTTTCTTGGTTTGGAGATGGCAGTCCCAAACAC
CAGAGTCTGTACTTTTCTATGACACAGCTCAAATTAAAGTAGGGCACAT
GCCAAGGAGGTTCTCACCTCCCTAAAGAAGGGACTTGAATTTTAGGGAC
TTTAATTCACCCTTTCCTTCAATCCAGCTTTCCCCCTTCTTGTTTGCAC
ATGCCAAGATAACTGCTTTTATGCAGGCTTTACCCCTGAAAAACTCCTT
TCTACAGTGCTGCTCACGAGAGCCCAGCCAGTCTCATGCCTGCATTGCT
GACTTGAACTCACAGTCGCCAAGTCTACCTATCTTTCTTGGAAGCAGTC
TTTCAAAATTCCTACTTGTATATTCATCCATTGTTGACAAGGACAAAAT
CAGTTGTATTTACAAAACTCTTAACTCGGTATTTTTTTTTTAAACTGAA
TCCTGTTTTGTAAATAATATACATTTAATGTTGCTTAGCATAAAAAATA
CTTTCTTACAGTAATCTTCTTTAAATTGGGTATATGAAAACATTACCAA
CTCTTGTTTTTCTATAAAGAAATACAGTTAATAACAAGGTTTCAAATTA
TTCCTTTAACATTAATAGAACAATCCAAATGTTGAGCATATTTTGATAT
TAAACTAAAGATCACGTCTCTGTTTTTAAATTATGCTAACACCACCACA
GATTATGTTGACGTATTTTCTTCTGTATTTTCTTGGTGGGACTTGAGAT
TTGAGGAAAAAAGATGCTTATTTGGAATTTCTATTAAGAACTATCCTTG
TTATTTTCTTAGCTCAATAATTACTCAAGAAAAATAAAAGATTATAATC
TCAGTTTAAAATTACTAATGTGTGACAACCATACCTCAGTTTGAAATAG
ATTTAACAAGATTATTCCCTGACATTTTCTTCCATGTAGCCATAAGCAG
GGACCCAGCATACAATTCTTCTTACCTAGGAATCAGGAACTAGTCCCAA
ATATACAGGATTGAGTCATGGTTTGCTTTTTTTTTTTTTCCTTTCCTTT
TTTTCTCCCCTTTACACATCAGATTTTGCTCAAATAGGAGGTAACTAAA
TAGCCATTAGAAGAAGAAGTGAAGCCTTTCATGCAGGATGATAGATCTA
ACTTATCAATGTACTGTCACAGTACATTAATCGTTTTATTTGAGTAGGT
AAACAAATTTGCACACTTTTCCCCAGTGAATTCCCAACACCTTGACTTC
CTCATTTCAGTGCTAATAACCTGTTTTGTGTGCAGTTAATCCATTCTGA
AGCAGGTGAAAGGTCAGTATTTTAGCATTTTAAAAGATGAGTTATATAA
TGTCTGCTTCCAGCCAGTCTGCAACATCTAGCCATACCTTTCTTATGCA
AGCCATTCAGTTATCAGGACTGTGAATTAACACTGTATGAATACATTTC
TGTACACCTTATTGTTTGGCCAGAGGGCCACCAAGTGTACTTATATGTA
ATCCTTAAATTTTAAACTAGCTTGTGATTTTTAAATATTTCTAAACTTT
TCTTAAACCACTAAAATTAAGCTCTTACTACTTAGTCAACTATCCTCAG
CTGTATTCGTCTTCAATTGTCAGTATGGCACAGATCACTGTATTAAAAT
GCTCTTTCGTCTTCATATTTACCTTCTGAGGTCATTTTTTAACTTAATG
TGTTACTACAAAGATTTGCAGATCTTTAATCAAGCACTATGTTAATACT
GTAATATCAAAATACTATGTCGAATTATTTCAGATGTTCAGATTGAATA
GGGTAAAAGAAAGTCTTTAAATGCTATCGCATCCTAGACCCTCTTCCCA
CCTACTATGGCATTGCATATTAGTCTGTTGAGACATTCATTGTTGCTTG
ACTGACATTTTGGTCTGGGTTTTCTTTTAATATTTTATTTTAATTAAAA
TTAGGAAAAAATGTGTTCAACAATTTTTTTTTTGTCCCATCTTCTCCAA
ATCAGTGGGAAAAGCTTACCTTTTGGTATATCTTAGTGTTTTCATGGGC
CCAGAACTGTGGTTTAAATTTTTATATGTATTCTCATTCTCATATTCAC
TCATACTCACTCTTTTTCTCTCTCTCCCTCTCTCTCCCTCTCTCCCCCC
CCCTTCTCTTTTGTGGAGTATAAATTAAAAAAACTGGATTTGGGACCTA
CAATACTCCTCAGGTTGAGATATTCTTGAAGTTTTAAAATAGCTTTAGT
TTTTAAAGTAAACTGGATTTGTGGACCTTAAAGTTACTGAGTTTAAACT
ACAAATTGTAACATCATTACTGAACATGTCCGCATCAACCCTCTGAGAA
TAACTTGGTCACTTTATGAAGGGGCGTTTTTAAGTTGTTTAGCAGTGAC
ATTTAATATGGTCCAATTGCTTTTCTTTTTAACATGACAAACAATTTTA
AAAGAGACTAAGGAACAAACACTATTGCGCCGAATGCCATAACACTGAG
TTGTACAAATTGTGATTGAGGAGATGAAAAAGTTTATACTTTTTAAAAA
AAAAAAAAACAAAAAACAAAACTTCAAATGGAATAAATTATTCATGAAG
CCTTC

In some embodiments, the oligonucleotide may have region of complementarity to a disease allele of MED13, for example as reported in Snijders Blok L., et. al, “De novo mutations in MED13, a component of the Mediator complex, are associated with a novel neurodevelopmental disorder” Hum. Genet. 2018, 137:375-388.

In some embodiments, an oligonucleotide comprises a region of complementarity to a MED13 sequence as set forth in SEQ ID NO: 888 or SEQ ID NO: 889. In some embodiments, the oligonucleotide comprises a region of complementarity that is at least 80%, 85%, 90%, 95%, 96%, 97%, 98%, 99%, or 100% complementary to a MED13 sequence as set forth in SEQ ID NO: 888 or SEQ ID NO: 889. In some embodiments, the oligonucleotide comprises a sequence that has at least 10, 11, 12, 13, 14, 15, 16, 17, 18, or 19 consecutive nucleotides that are perfectly complementary to a MED13 sequence as set forth in SEQ ID NO: 888 or SEQ ID NO: 889. In some embodiments, an oligonucleotide may comprise a sequence that targets (e.g., is complementary to) an RNA version (i.e., wherein the T's are replaced with U's) of a MED13 sequence as set forth in SEQ ID NO: 888 or SEQ ID NO: 889. In some embodiments, the oligonucleotide comprises a sequence that is complementary (e.g., at least 80%, 85%, 90%, 95%, 96%, 97%, 98%, 99%, or 100% complementary) to an RNA version of a MED13 sequence as set forth in SEQ ID NO: 888 or SEQ ID NO: 889. In some embodiments, the oligonucleotide comprises a sequence that has at least 10, 11, 12, 13, 14, 15, 16, 17, 18, or 19 consecutive nucleotides that are perfectly complementary to an RNA version of a MED13 sequence as set forth in SEQ ID NO: 888 or SEQ ID NO: 889.

In some embodiments, a MED13-targeting oligonucleotide comprises an antisense strand that comprises at least 10, 11, 12, 13, 14, 15, 16, 17, 18, or 19 consecutive nucleotides of a sequence comprising any one of SEQ ID NOs: 938-961. In some embodiments, a MED13-targeting oligonucleotide comprises an antisense strand that comprises any one of SEQ ID NOs: 938-961. In some embodiments, a MED13-targeting oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 938-961.

In some embodiments, a MED13-targeting oligonucleotide comprises an antisense strand that targets a MED13 sequence comprising any one of SEQ ID NOs: 890-937. In some embodiments, an oligonucleotide comprises an antisense strand comprising at least 10, 11, 12, 13, 14, 15, 16, 17, 18, or 19 nucleotides (e.g., consecutive nucleotides) that are complementary to a MED13 sequence comprising any one of SEQ ID NOs: 890-937. In some embodiments, a MED13-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NOs: 890-937.

In some embodiments, a MED13-targeting oligonucleotide comprises an antisense strand comprises a region of complementarity to a target sequence as set forth in any one of SEQ ID NOs: 890-937. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a MED13-targeting oligonucleotide further comprises a sense strand that hybridizes to the antisense strand to form a double stranded siRNA. In some embodiments, the MED13-targeting oligonucleotide comprises an antisense strand that comprises the nucleotide sequence of any one of SEQ ID NOs: 938-961. In some embodiments, the MED13-targeting oligonucleotide further comprises a sense strand that comprises the nucleotide sequence of any one of SEQ ID NOs: 914-937.

In some embodiments, the MED13-targeing oligonucleotide is a double stranded oligonucleotide (e.g., an siRNA) comprising an antisense strand that comprises the nucleotide sequence of any one of SEQ ID NOs: 938-961 and a sense strand that hybridizes to the antisense strand and comprises the nucleotide sequence of any one of SEQ ID NOs: 914-937, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the MED13-targeing oligonucleotide is a double stranded oligonucleotide (e.g., an siRNA) comprising an antisense strand that comprises the nucleotide sequence of any one of SEQ ID NOs: 938-961 and a sense strand that hybridizes to the antisense strand and comprises the nucleotide sequence of any one of SEQ ID NOs: 914-937, wherein the each nucleoside in the antisense strand and/or (e.g., and) each nucleoside in the sense strand is a 2′-modified nucleoside selected from 2′-O-Me and 2′-F modified nucleosides.

In some embodiments, the MED13-targeing oligonucleotide is a double stranded oligonucleotide (e.g., an siRNA) comprising an antisense strand that comprises the nucleotide sequence of any one of SEQ ID NOs: 938-961 and a sense strand that hybridizes to the antisense strand and comprises the nucleotide sequence of any one of SEQ ID NOs: 914-937, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the MED13-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the MED13-targeing oligonucleotide comprises a structure of (5′ to 3′):
mNmNfNmNfNmNfNmNfNmNfNmNfNmNfNmNfNmNfNmNfN, wherein “mN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the MED13-targeing oligonucleotide is selected from the modified version of SEQ ID NOs: 938-961 listed in Table 37. In some embodiments, the sense strand of the MED13-targeing oligonucleotide is selected from the modified version of SEQ ID NOs: 914-937 listed in Table 37. In some embodiments, the MED13-targeing oligonucleotide is a siRNA selected from the siRNAs listed in Table 37.

TABLE 35
MED13 Target Sequences

| | Corre- | | |
|---|---|---|---|
| Reference | sponding | | |
| sequence | first | | |
| NM_ | nucleotide | | |
| 005121.3 | of | MED13 Target | SEQ |
| (SEQ ID | Reference | Sequence | ID |
| NO: 888) | Sequence | (5′ to 3′) | NO: |
| NM_005121.3 | 351 | ACCTTATTCACCATGACTT | 890 |
| NM_005121.3 | 1018 | AAGAGATCCTGCTATGTCT | 891 |
| NM_005121.3 | 1109 | GTCAAATTTTCTTCAGTAT | 892 |
| NM_005121.3 | 2143 | ACAAGAACCTAAAATTGAT | 893 |
| NM_005121.3 | 2296 | AGAAGATGCTATGTCATTA | 894 |
| NM_005121.3 | 3154 | TGCTAGTGCTCAAGGTTCA | 895 |
| NM_005121.3 | 4006 | ACAATTTCATAAAATGGCT | 896 |
| NM_005121.3 | 5851 | GCCAGATTCTGTGTCAACT | 897 |
| NM_005121.3 | 6380 | CACGTGCCTTCAGTGCAAT | 898 |
| NM_005121.3 | 6444 | ATCAGACTTCAGATGTCCT | 899 |
| NM_005121.3 | 6722 | AACAAATATTGTTTTGACT | 900 |
| NM_005121.3 | 7056 | TAAATGTATAAATTGTCTT | 901 |
| NM_005121.3 | 7070 | GTCTTATATTTCTTAACCT | 902 |
| NM_005121.3 | 7098 | AAATTTTCCTAGAAGGTTT | 903 |
| NM_005121.3 | 7147 | CTAAAATATATGTAATCGT | 904 |
| NM_005121.3 | 1987 | CAAGGATGATCCAGTTGGA | 905 |
| NM_005121.3 | 6552 | TTGTGGTGCTGAATCAGTT | 906 |
| NM_005121.3 | 303 | GAAGAGAATTGTGGATATT | 907 |
| NM_005121.3 | 280 | GCGGCGAGATCAAAGACCT | 908 |
| NM_005121.3 | 1555 | CCAAAGACTTGTGATCTCT | 909 |
| NM_005121.3 | 4173 | GAGATATAGCCTATGTTGT | 910 |
| NM_005121.3 | 1186 | AGCAAATCATGTGGTGGAT | 911 |
| NM_005121.3 | 4027 | CCGAGGCTCTTATGGAACT | 912 |
| NM_005121.3 | 5454 | ACTGTTTATCACATGATCA | 913 |

* The target sequences contain Ts, but binding to RNA and/or DNA is contemplated.

In some embodiments, an oligonucleotide may comprise or consist of any sequence as provided in Table 36.

TABLE 36
Oligonucleotide sequences for targeting MED13

| Passenger Strand/ | | Guide Strand/ | |
|---|---|---|---|
| Sense Strand | SEQ | Antisense | SEQ |
| (RNA) | ID | Strand (RNA) | ID |
| (5′ to 3′) | NO: | (5′ to 3′) | NO: |
| UGACCUUAUUCACCAUGACUU | 914 | AAGUCAUGGUGAAUAAGGUCAGC | 938 |
| ACAAGAGAUCCUGCUAUGUCU | 915 | AGACAUAGCAGGAUCUCUUGUGG | 939 |
| GGGUCAAAUUUUCUUCAGUAU | 916 | AUACUGAAGAAAAUUUGACCCAC | 940 |
| GAACAAGAACCUAAAAUUGAU | 917 | AUCAAUUUUAGGUUCUUGUUCUG | 941 |
| GAAGAAGAUGCUAUGUCAUUA | 918 | UAAUGACAUAGCAUCUUCUUCAU | 942 |
| CCUGCUAGUGCUCAAGGUUCA | 919 | UGAACCUUGAGCACUAGCAGGUC | 943 |
| CAACAAUUUCAUAAAAUGGCU | 920 | AGCCAUUUUAUGAAAUUGUUGCC | 944 |
| AUGCCAGAUUCUGUGUCAACU | 921 | AGUUGACACAGAAUCUGGCAUAA | 945 |
| UCCACGUGCCUUCAGUGCAAU | 922 | AUUGCACUGAAGGCACGUGGAGG | 946 |
| AAAUCAGACUUCAGAUGUCCU | 923 | AGGACAUCUGAAGUCUGAUUUGA | 947 |
| UCAACAAAUAUUGUUUUGACU | 924 | AGUCAAAACAAUAUUUGUUGAAG | 948 |
| UUUAAAUGUAUAAAUUGUCUU | 925 | AAGACAAUUUAUACAUUUAAAAA | 949 |
| UUGUCUUAUAUUUCUUAACCU | 926 | AGGUUAAGAAAUAUAAGACAAUU | 950 |
| AAAAAUUUUCCUAGAAGGUUU | 927 | AAACCUUCUAGGAAAAUUUUUAU | 951 |
| GUCUAAAAUAUAUGUAAUCGU | 928 | ACGAUUACAUAUAUUUUAGACCA | 952 |
| UUCAAGGAUGAUCCAGUUGGA | 929 | UCCAACUGGAUCAUCCUUGAAUU | 953 |
| UUUUGUGGUGCUGAAUCAGUU | 930 | AACUGAUUCAGCACCACAAAAUG | 954 |
| AAGAAGAGAAUUGUGGAUAUU | 931 | AAUAUCCACAAUUCUCUUCUUCC | 955 |
| UGGCGGCGAGAUCAAAGACCU | 932 | AGGUCUUUGAUCUCGCCGCCAAA | 956 |
| AGCCAAAGACUUGUGAUCUCU | 933 | AGAGAUCACAAGUCUUUGGCUGG | 957 |
| AAGAGAUAUAGCCUAUGUUGU | 934 | ACAACAUAGGCUAUAUCUCUUUG | 958 |
| UUAGCAAAUCAUGUGGUGGAU | 935 | AUCCACCACAUGAUUUGCUAAUU | 959 |
| GGCCGAGGCUCUUAUGGAACU | 936 | AGUUCCAUAAGAGCCUCGGCCAG | 960 |
| AUACUGUUUAUCACAUGAUCA | 937 | UGAUCAUGUGAUAAACAGUAUCC | 961 |

In some embodiments, an oligonucleotide is a modified oligonucleotide as provided in Table 37, wherein ‘inN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 37
Modified Oligonucleotides for targeting MED13

| | Modified Passenger | | Modified Guide | |
|---|---|---|---|---|
| | Strand/Sense | SEQ | Strand/Antisense | SEQ |
| | Strand (RNA) | ID | Strand (RNA) | ID |
| siRNA # | (5′ to 3′) | NO: | (5′ to 3′) | NO: |
| hsMED13-1 | mUmGfAmCfCmUfUmAfU | 914 | fAfAmGfUmCfAmUfGmGfUm | 938 |
| | mUfCmAfCmCfAmUfGmAf | | GfAmAfUmAfAmGfGmUfCmA | |
| | CmUfU | | *fG*mC | |
| hsMED13-2 | mAmCfAmAfGmAfGmAfU | 915 | fAfGmAfCmAfUmAfGmCfAmG | 939 |
| | mCfCmUfGmCfUmAfUmGf | | fGmAfUmCfUmCfUmUfGmU*f | |
| | UmCfU | | G*mG | |
| hsMED13-3 | mGmGfGmUfCmAfAmAfU | 916 | fAfUmAfCmUfGmAfAmGfAm | 940 |
| | mUfUmUfCmUfUmCfAmGf | | AfAmAfUmUfUmGfAmCfCmC | |
| | UmAfU | | *fA*mC | |
| hsMED13-4 | mGmAfAmCfAmAfGmAfA | 917 | fAfUmCfAmAfUmUfUmUfAm | 941 |
| | mCfCmUfAmAfAmAfUmUf | | GfGmUfUmCfUmUfGmUfUmC | |
| | GmAfU | | *fU*mG | |
| hsMED13-5 | mGmAfAmGfAmAfGmAfU | 918 | fUfAmAfUmGfAmCfAmUfAm | 942 |
| | mGfCmUfAmUfGmUfCmAf | | GfCmAfUmCfUmUfCmUfUmC | |
| | UmUfA | | *fA*mU | |
| hsMED13-6 | mCmCfUmGfCmUfAmGfU | 919 | fUfGmAfAmCfCmUfUmGfAmG | 943 |
| | mGfCmUfCmAfAmGfGmUf | | fCmAfCmUfAmGfCmAfGmG*f | |
| | UmCfA | | U*mC | |
| hsMED13-7 | mCmAfAmCfAmAfUmUfU | 920 | fAfGmCfCmAfUmUfUmUfAmU | 944 |
| | mCfAmUfAmAfAmAfUmG | | fGmAfAmAfUmUfGmUfUmG*f | |
| | fGmCfU | | C*mC | |
| hsMED13-8 | mAmUfGmCfCmAfGmAfU | 921 | fAfGmUfUmGfAmCfAmCfAmG | 945 |
| | mUfCmUfGmUfGmUfCmAf | | fAmAfUmCfUmGfGmCfAmU*f | |
| | AmCfU | | A*mA | |
| hsMED13-9 | mUmCfCmAfCmGfUmGfC | 922 | fAfUmUfGmCfAmCfUmGfAmA | 946 |
| | mCfUmUfCmAfGmUfGmCf | | fGmGfCmAfCmGfUmGfGmA*f | |
| | AmAfU | | G*mG | |
| hsMED13-10 | mAmAfAmUfCmAfGmAfC | 923 | fAfGmGfAmCfAmUfCmUfGmA | 947 |
| | mUfUmCfAmGfAmUfGmU | | fAmGfUmCfUmGfAmUfUmU*f | |
| | fCmCfU | | G*mA | |
| hsMED13-11 | mUmCfAmAfCmAfAmAfU | 924 | fAfGmUfCmAfAmAfAmCfAmA | 948 |
| | mAfUmUfGmUfUmUfUmG | | fUmAfUmUfUmGfUmUfGmA*f | |
| | fAmCfU | | A*mG | |
| hsMED13-12 | mUmUfUmAfAmAfUmGfU | 925 | fAfAmGfAmCfAmAfUmUfUm | 949 |
| | mAfUmAfAmAfUmUfGmU | | AfUmAfCmAfUmUfUmAfAmA | |
| | fCmUfU | | *fA*mA | |
| hsMED13-13 | mUmUfGmUfCmUfUmAfU | 926 | fAfGmGfUmUfAmAfGmAfAm | 950 |
| | mAfUmUfUmCfUmUfAmA | | AfUmAfUmAfAmGfAmCfAmA | |
| | fCmCfU | | *fU*mU | |
| hsMED13-14 | mAmAfAmAfAmUfUmUfU | 927 | fAfAmAfCmCfUmUfCmUfAmG | 951 |
| | mCfCmUfAmGfAmAfGmGf | | fGmAfAmAfAmUfUmUfUmU*f | |
| | UmUfU | | A*mU | |
| hsMED13-15 | mGmUfCmUfAmAfAmAfU | 928 | fAfCmGfAmUfUmAfCmAfUmA | 952 |
| | mAfUmAfUmGfUmAfAmU | | fUmAfUmUfUmUfAmGfAmC*f | |
| | fCmGfU | | C*mA | |
| hsMED13-16 | mUmUfCmAfAmGfGmAfU | 929 | fUfCmCfAmAfCmUfGmGfAmU | 953 |
| | mGfAmUfCmCfAmGfUmUf | | fCmAfUmCfCmUfUmGfAmA*f | |
| | GmGfA | | U*mU | |
| hsMED13-17 | mUmUfUmUfGmUfGmGfU | 930 | fAfAmCfUmGfAmUfUmCfAmG | 954 |
| | mGfCmUfGmAfAmUfCmAf | | fCmAfCmCfAmCfAmAfAmA*f | |
| | GmUfU | | U*mG | |
| hsMED13-18 | mAmAfGmAfAmGfAmGfA | 931 | fAfAmUfAmUfCmCfAmCfAmA | 955 |
| | mAfUmUfGmUfGmGfAmU | | fUmUfCmUfCmUfUmCfUmU*f | |
| | fAmUfU | | C*mC | |
| hsMED13-19 | mUmGfGmCfGmGfCmGfA | 932 | fAfGmGfUmCfUmUfUmGfAm | 956 |
| | mGfAmUfCmAfAmAfGmA | | UfCmUfCmGfCmCfGmCfCmA* | |
| | fCmCfU | | fA*mA | |
| hsMED13-20 | mAmGfCmCfAmAfAmGfA | 933 | fAfGmAfGmAfUmCfAmCfAmA | 957 |
| | mCfUmUfGmUfGmAfUmCf | | fGmUfCmUfUmUfGmGfCmU*f | |
| | UmCfU | | G*mG | |
| hsMED13-21 | mAmAfGmAfGmAfUmAfU | 934 | fAfCmAfAmCfAmUfAmGfGmC | 958 |
| | mAfGmCfCmUfAmUfGmUf | | fUmAfUmAfUmCfUmCfUmU*f | |
| | UmGfU | | U*mG | |
| hsMED13-22 | mUmUfAmGfCmAfAmAfU | 935 | fAfUmCfCmAfCmCfAmCfAmU | 959 |
| | mCfAmUfGmUfGmGfUmG | | fGmAfUmUfUmGfCmUfAmA*f | |
| | fGmAfU | | U*mU | |
| hsMED13-23 | mGmGfCmCfGmAfGmGfC | 936 | fAfGmUfUmCfCmAfUmAfAmG | 960 |
| | mUfCmUfUmAfUmGfGmA | | fAmGfCmCfUmCfGmGfCmC*f | |
| | fAmCfU | | A*mG | |
| hsMED13-24 | mAmUfAmCfUmGfUmUfU | 937 | fUfGmAfUmCfAmUfGmUfGm | 961 |
| | mAfUmCfAmCfAmUfGmAf | | AfUmAfAmAfCmAfGmUfAmU | |
| | UmCfA | | *fC*mC | |

l. PPP1R3A Oligonucleotides

Examples of oligonucleotides for targeting PPP1R3A are described in Cordero P, et al., Pathologic gene network rewiring implicates PPP1R3A as a central regulator in pressure overload heart failure. Nat Commun. 2019 Jun. 24; 10(1):2760. doi: 10.1038/s41467-019-10591-5; the contents of which are incorporated herein in their entirety.

In some embodiments, oligonucleotides may have a region of complementarity to a human PPP1R3A sequence, for example, as provided below by Gene ID: 5506; NCBI Ref. No: NM_002711.4:

(SEQ ID NO: 962)
ATCAGAGAGCCCAATGGAGCCTTCTGAAGTACCTAGTCAGATTAG
CAAAGATAATTTTTTAGAAGTTCCTAATTTATCTGACTCTCTTTG
TGAAGATGAAGAAGTTACTTTCCAACCTGGTTTCTCCCCTCAACC
AAGTAGACGAGGTTCTGATTCTTCTGAAGACATATACCTGGATAC
CCCATCTTCAGGTACTAGAAGAGTTTCATTTGCTGATTCCTTTGG
ATTCAATCTTGTGTCTGTTAAAGAATTTGATTGCTGGGAATTACC
GAGTGCTTCAACCACTTTTGACTTAGGGACGGACATTTTCCACAC
AGAAGAATATGTTTTAGCCCCACTGTTTGACTTGCCTTCTTCAAA
AGAAGATCTTATGCAACAACTCCAAATACAGAAAGCAATACTGGA
GTCAACTGAGTCTCTTCTTGGGTCTACAAGTATCAAGGGTATTAT
TCGAGTTTTGAATGTTTCTTTTGAGAAGTTAGTATATGTAAGAAT
GTCTTTAGATGACTGGCAGACACATTATGACATTTTAGCAGAATA
TGTTCCTAATTCATGTGATGGTGAAACTGACCAGTTCTCCTTTAA
GATTGTATTGGTTCCTCCTTATCAAAAAGATGGCAGTAAAGTTGA
GTTTTGTATACGTTATGAAACTTCTGTTGGTACATTTTGGTCAAA
TAATAATGGCACAAATTATACATTCATTTGTCAAAAGAAAGAACA
AGAGCCGGAGCCTGTAAAACCATGGAAAGAAGTTCCTAACAGACA
AATAAAAGGCTGCTTAAAGGTAAAATCAAGTAAAGAAGAATCATC
AGTAACATCAGAAGAAAATAACTTTGAGAATCCAAAGAATACAGA
TACCTATATCCCAACAATCATTTGTTCTCATGAGGACAAGGAAGA
TTTGGAAGCCAGTAATCGAAATGTAAAAGATGTAAACAGGGAACA
TGATGAACATAATGAAAAAGAATTAGAGTTGATGATAAATCAACA
CTTAATAAGAACCAGAAGTACTGCTTCCAGAGATGAAAGGAATAC
ATTTTCAACAGATCCAGTCAATTTTCCAAATAAAGCAGAGGGGTT
AGAGAAGAAGCAAATCCATGGTGAAATATGTACTGACTTGTTCCA
AAGGTCTCTGTCTCCAAGTTCATCAGCAGAAAGCTCCGTAAAGGG
AGATTTTTACTGCAATGAAAAATATTCCTCAGGAGATGACTGTAC
ACATCAACCTTCAGAGGAAACTACTTCAAATATGGGAGAAATCAA
GCCATCATTGGGAGATACTAGTAGTGATGAACTAGTGCAATTACA
TACTGGCAGCAAAGAAGTCCTGGATGATAATGCTAATCCAGCCCA
TGGCAATGGCACAGTGCAAATACCTTGCCCCTCTTCAGATCAACT
AATGGCAGGAAACCTTAATAAAAAACATGAAGGAGGAGCTAAAAA
TATTGAAGTAAAAGATTTGGGATGTTTACGAAGAGATTTCCATTC
AGATACGTCGGCATGTCTCAAAGAATCAACAGAAGAAGGATCTTC
TAAGGAAGATTATTATGGCAATGGTAAGGATGATGAAGAACAAAG
AATATATTTAGGTGTTAATGAAAAACAAAGAAAAAATTTCCAAAC
AATCTTACATGACCAAGAAAGGAAGATGGGTAACCCTAAAATAAG
TGTGGCAGGGATTGGAGCTAGTAACAGAGACCTGGCTACTCTGCT
GAGCGAACATACCGCAATCCCCACCCGGGCAATCACAGCAGATGT
GTCTCATTCACCAAGGACAAATTTAAGTTGGGAAGAAGCTGTGTT
AACCCCAGAGCATCATCATTTGACTAGTGAAGGCAGCGCTTTAGG
AGGGATAACTGGTCAAGTTTGTTCATCAAGAACTGGAAATGTTTT
GAGGAATGATTATCTTTTCCAAGTTGAAGAAAAATCAGGTGGGAT
TAATTCTGAAGATCAGGATAATAGCCCACAGCATAAACAAAGTTG
GAATGTTCTGGAAAGTCAGGGAAAATCAAGAGAGAATAAGACAAA
CATAACAGAGCATATCAAAGGACAAACAGATTGTGAAGACGTGTG
GGGAAAAAGAGATAATACGAGGAGTTTGAAAGCTACTACAGAAGA
ATTGTTTACCTGCCAAGAAACAGTGTGCTGTGAACTGTCTTCTCT
AGCTGATCATGGCATTACTGAGAAAGCAGAAGCTGGTACAGCCTA
TATAATTAAGACAACATCAGAAAGTACTCCAGAAAGCATGTCTGC
TAGAGAAAAAGCAATAATTGCTAAGCTACCTCAAGAGACAGCACG
AAGTGACAGGCCCATCGAGGTAAAGGAAACAGCGTTTGATCCACA
TGAAGGGAGAAATGATGATTCACATTATACCCTTTGTCAACGAGA
TACAGTAGGTGTAATCTATGACAATGATTTTGAAAAGGAATCACG
TTTAGGTATTTGTAATGTACGTGTAGATGAAATGGAGAAGGAAGA
AACCATGTCTATGTACAATCCTAGGAAGACACATGACAGGGAGAA
ATGTGGCACTGGAAATATAACATCTGTGGAAGAATCCTCATGGGT
CATTACAGAATATCAAAAAGCAACTTCAAAACTGGATTTACAGTT
GGGAATGTTACCAACAGACAAAACTGTATTTTCAGAAAACAGAGA
TCTTAGGCAGGTTCAAGAATTATCAAAGAAAACAGACTCGGATGC
CATTGTGCATTCTGCTTTTAACTCAGACACTAATAGAGCTCCTCA
GAATAGCTCTCCTTTTTCCAAACATCATACTGAAATTTCAGTGTC
AACTAATGAGCAGGCAATTGCTGTAGAGAATGCAGTTACTACCAT
GGCTAGCCAACCTATTTCTACGAAATCAGAAAATATTTGTAATTC
AACAAGAGAAATCCAGGGTATTGAGAAGCACCCTTATCCTGAGTC
TAAACCTGAAGAAGTTTCCAGAAGTTCAGGAATAGTGACATCAGG
TAGTAGAAAAGAAAGATGCATAGGCCAGATTTTCCAAACAGAAGA
GTATAGTGTGGAAAAATCTCTAGGGCCAATGATTTTAATCAACAA
ACCTCTTGAGAATATGGAAGAAGCAAGGCATGAAAATGAAGGATT
AGTAAGCTCTGGGCAATCACTATACACTTCAGGTGAAAAGGAATC
TGACAGCTCTGCTTCTACTAGTCTTCCTGTTGAGGAAAGTCAAGC
TCAAGGCAACGAATCTTTGTTTTCAAAATATACCAACTCTAAAAT
ACCTTATTTCCTTTTGTTTCTGATATTTCTTATAACTGTCTACCA
TTATGACTTAATGATTGGCTTGACATTCTACGTTTTGTCATTGTC
CTGGCTATCCTGGGAAGAGGGTAGACAAAAAGAGTCTGTCAAAAA
GAAGTAACCTCAGCACTACTATTCTCTCTTAAAAGATAAGCTATT
TAACCCCAAACATTTGGATTGGTGAATGGGACTATTCATTGTTCA
AAGATCCAGTGCAGTTTTTCTCTTGAAGGATCATTTAAAAAGGGA
CGCGAATAAGTTTGCTCCTTCATATAAGTAATTATTCTATATAGG
ACCATTATGTTTGGATCATTAAATACCTATATGAATATGAGATCT
GAAGCACGTCAAGTTGAAATTAGGTACAGCTGTTGCTCCTTAGCA
GGCTATGAAGTTGCAATGCTTCACATCTCTTCACTACTTAAAGTG
CTATTTCTTGCATTCATTTCTCTTGCAATAAAGCTTCATTTTTTT
TTCCCCTTGAGAGTATATATTTTCCCCTACGGTTTTAAAAAACAG
ATAAAACATGGACAATGGCAGAGGACTTTTTTGGTCTTTTAGTAT
TGAACATGAAATTTGTTCAATTTATCATTTATCAGGATTCATTGA
TCAAATATTTCAACCTTTCATATTTTTAAGAAAACACCCATATAT
ATTGAAATGCAAACTTAAACATATTCTGTTCACTTGAGTGTAATA
CTTGATGCATGCACACACACACACACATACCTTCTTTATAGCTAC
AAAGTCAGGGCGTTCATAGGCAAAATCTGACAAGACTGAAACAAT
TGGGAGATTATCTTTCAATTCTGAAAAATTCTGTGGGTGGCCTGT
CTTTAAGCAATGCTTTTCTCTTGTGAGGGAAGACTGTAACACAGC
TGAACTATTTCAGTGTCAACTATTCAGAATTGAAAATGTAAATTA
AAATTTCCACAAGCATTGCTTCAGAATGAATTTGTACCTATAAAG
CATAAGGCATTAAATGACAATAAAAATTCCAAATGGACTATTTGT
TCCACATTGTATTTTTGTCTTCAAATATTTTCTAAGAGAATGAAT
TATCCCCC

In some embodiments, oligonucleotides may have a region of complementarity to a mouse PPP1R3A sequence, for example, as provided below by Gene ID: 140491; NCBI Ref. No: NM_080464.2:

(SEQ ID NO: 963)
ACTCTTATTTGATAAATCAGAGAGACCAATGGAGCCCGCTGAAGA
ACCTGGTCAGATTAGCAAAGATAACTTTTTAGAAGTTCCTAATCT
GTCTGATTCTGTGTGTGAGGATGAAGAAGTTAAGGCGACCTTCAA
GCCTGGCTTCTCCCCTCAGCCAAGCAGACGCGGGTCAGGATCTTC
GGAAGACATGTATCTGGACACTCCAACCTCAGCTTCCCGGAGAGT
TTCCTTTGCTGACAGCCTGGGATTCAGCCTTGTGTCTGTTAAAGA
ATTTGATTGCTGGGAACTACCCAGTGTTTCAACTGATTTTGACTT
AAGTGGGGATGTTTTCCACACAGATGAATATGTTTTATCTCCACT
GTTTGACTTGCCTTCTTCAAAAGAAAAGCTTATGGAACAACTCCA
AGTCCAGAAAGCCGTGCTGGAGTCCGCTGAACACCTTCCCGGGTC
AAGCATGAAGGGCATTATTCGAGTCCTGAACATTTCTTTTGAGAA
GTTAGTGTATGTGCGCATGTCTTTGGATGACTGGCAGACACATTA
TGACATTTTAGCAGAATATGTTCCTAACTCGTGCGACGGTGAGAC
TGACCAATTCTCTTTTAAGATTTCATTGGTTCCACCTTATCAAAA
AGAGGGCGGTAAAGTGGAGTTTTGTATACGCTATGAAACTTCTGC
TGGCACATTTTGGTCAAATAATAATGGCACAAATTATATATTGGT
TTGTCAAAAGAAGAGGAAGGAGCCAGAGCCTGTAAAGCCATTGGA
AGAAGCACCTAGCAGACAAATAAAAGGCTGCTTAAAGGTAAAATC
ACGCAGTAAAGAAGAACCATTGCTAGCACCAGAAGAAAATAAATT
TGAGACTTTAAAGTTCACAGAGTCCTACATCCCAACAATCATTTG
TTCTCACGAGGACAAGGATGACTTGGGAGCCAATCATCCAAATGT
AGACGATATCAACAAGAAGCATGACGAACACAATGGAAAGGAGCT
AGACTTGATGATAAACCAACGGTTAATAACTTCCCAGGATGAAAA
GAATACATTTGCAACAGATACAGTCAATTTTACAAATAAAGCTGA
AGGGTCAGAGAAGAAGCAAGCTTACCATGAAATCAACACTGACTT
GTTCATGGGGCCTTTGTCTCCAAGTTTATCAGCAGAAAGTTCCTT
AAAGCGAGATTTCTATCATAGCAGAAGTTCTTCCCCAGGAAATGA
ATATGGTCATCCACATTCAGAAGAAATTATTTCAGATATGGGAGA
AAAAGGTCCATCACTGGGAGACACTAGTTCTGATGAGTTAATGCA
ATTGGAACTCTGCAGCAAGGAAGACCTGGATGATAATGCCAACCC
AGCCAATGGGAGTGGCAGAGTGTGCTCTTCCTTCGATCAAAGAAT
GGCATGTGGTCTCAAGAATAATGAAGCAGGAATCAAGAAAACTGG
AATACAAGATTACAAATATTCACATGGAGATTCCACAAAATTGGA
AGAGTCAAATGCATCTTCCAGAGATGATTATGCCAAAGTCGATAA
TAAAAAGGAGAAGCAAACATGCTTAGGTGTTAATGAAAACCCAAG
CAAGAATTTTCAATCAGTATTCCAGACCCAAGAGGGACACATGGG
GTACCCAAAAATAAGCACTGAAGGGGATAAAGCTAATAACCAAGA
CTTGACAAGTCTACTGAGCAAAGATATTACAGCTAACACCTGGGC
GGTGACAGTGGATCCCTGTCCTTCCACAAATGCAAAAAGGAGTTG
GAGAGAAGTTGGAAGTGGTAGCAATTTAGAACCAGGAACTAGTGA
TCTTAGTTCTCCAAGGAATTTCAGCCCTCTGACTGATGATCATCT
TTTTCAAGCTGACAGGGAAAACTCAGACTCTAGCAATCCTGAAAA
TCAGAATATGAACACACGACATAGAAAAAAATGGAACGTTCTGGA
AACCCAGTCAGAAACAAGTGAGACTGAATCAGATATAGCCAAGCA
TACCAAAGAACAAGCAGAGTATAAAGACATGTGGGAAAAAACAGA
TAACAGTAGGAATCTGAAAGCTACTCCTACAGAACACTTGTTTAC
CTGCCGAGAGACAGAATGCTACGGACTGTCTTCTCTTGCTGATCA
TGGTATTACTGAGAAAGCACAAGCAGTTACAGCTTATATAATTAA
GACAACATTAGAAAGTACTCCAGAAAGCGCGTCTGCTAGAGGAAA
AGCAATAATTGCTAAGCTACCTCAAGAGACAGCAGGAAATGACAG
GCCCATCGAGGTAAAAGAAACAGCGTTTGATCCACATGAAGGGAG
GAAAGATGATTCACATTATTCCCTTTGTCATGGAGATACAGCAGG
TGTAATCCATGACAATGATTTTGAAAGGGAGTCACATTTAGATAT
TTGTAATTTACGTGTGGATGAAATGAAGAAGGAGAAAACCACATC
TACGTGCTTTCCTCAGAAGACATATGACAAGGAGAAACATGGAAT
TGGAAGTGTAACATCTATAGACGAACCTTCACAGGTCATTACAGG
CAATCAAAAAGCTACGTCAAAACTGGATTTACATTTGGGAGTGTT
ACCAACAGACAGAGCAATATTCCAAGCAAATGCAGATCTTGAGCT
GCTTCAAGAATTATCAAGGAGAACAGACTTCAACGCCGTTCCCTC
TGCATTTAACTCAGACACTGCCAGCGCTTCTCGGGACAGCTCTCA
AGTTTACAGACACTGCTCTAAAAAATCAGTACCTTCTTACGGTGA
AGAGAAAGCAGTTACTAACACAACCCTCCAGTCTATTCCTACCAA
ATCAGAATATAATTGGCACCCAGAAAGTGAGGTCTTGGGCCATGC
CATGTCTAAACCTGAAGATGTTTTCAAAAGTTCAGAAATAATGAA
ATCAGGTAGTGGAGGAGAACGAGGCGGGGGTCCGATTCTCCAACA
AAAAGAAGGTAGCTTGGAAAACTCCCAGGGCCCGATGTTTTTTAC
CAATGAGCCTCTTGAGAATCTGGATGAAGCAAGTTCTGAAAATGA
AGGGTTAATGCATTCTGGACAGTCACAGTGTTATCTGGGTGACAA
AGGATTAGTGAGCTCTGCTTCTGCTACTGTCTCTACGCAGGAACT
CGAAGCTCAAGGCAGGGAGTCTCTGCTTTCAATATCCACCAACTC
TAAAATACCATATTTCCTTTTGTTTCTGATATTTCTTGCAACTGT
CTACTACTATGACTTAATGATAGGCTTGGCGTTCTACCTTTTTTC
ATTATACTGGTTGTACTGGGAAGGGGGGAGGCAAAGAGAGTCTGT
TAAGAAAAAGTGAGCTTGGCACAGTTGCTATTCTCTCTTGAAAGA
TAAGCTATTTAGTCCCAAACATTTGGAATGGCAGGAGAGACTATT
CATTGTTCAAAGAATCAGTGCAGTTTTTCTCTTGAAGGATCATTT
AAAAAGGAATGCGTATGAAGTTTGCTCCTTCATATAAGTAATTAT
TCTATATAGGACCATTATGCTTGGATCATTAAATACCTATATGAA
TATGAGATGTGAAGCACGTCAAGTTGGAATTAGGTACAGCTGTTG
CTCCTTAGCAGGCTGTGAAGTTGAAATGCTTCATGTCTCTTCACT
ATTTAAAGTGCGATTTCCTGCAATCATTTCTTTCACAGTAAAGCT
TTGGATATTTTTTTTCCCTGAGAGCACTTTCCTCCTAGGTTTTTA
AAAAAATAGATAAAACATGGAGAGATATTTTTTCTTTTAGTCCCT
AGAATTTGCCATGAAAATTTCATTTAGCACTGTGTACTCTATCAG
CACATATTGATAGATCAAATATTAAAATTTCTGTTTATGCCTTTA
AGAAAATACCCATGTACTTTGCAATATGAAAACGTAAATTGGAAA
TATGTTTTATGCACACTCACATTATACTTTATGTGCATACCATTG
TATCTCTTTGTACCCATGATGTCAGGGTTTTCATACACCAATTTT
AACTTTGTTTGTTTATTTTTATTTAGTTGAATGTGTAGACTGTAA
CACAAATGGACAATTTTAATGTTAAGTGTTGTCCAGACATGAAAA
TTCTAACATTCTATCATCATTGTTTCAAGGTGATTTTGCACCTAC
CTTGTAGTTCTTAACCTGTGGGTTACAACCCCTTTGGAGGCTGCA
TATCAGATATTTATGTTATCATTCACAACAGTGAGCAGAATTACA
GTTATGAAGTATAAACAAAACAAACTTATGGCTGGGGTCACCACA
ACATGTGGCACTCTATTAAAGGGTCACAACATTAAAAAGGTTGAG
AACCACTGCTATAGAACATAAGGCCTTAAGTAATAATATAATAGA
TGCTAAAATGGTTTGTTCTTCCTGGTAGTTCCTTTGTTTTCGATT
AGTTGTTAATATTTTCTTATAGTAGGGAGATGAATTTTATCCTAA
GTAAATTTACAGAACAAACTCCTTGTCTGGTAATATCCATTTTGT
CATCTTTGGGTTATGAGACAATCACTCAAAACTTTAAGCAGGCAA
AATATGTATAGTTGAAAGCATACTGAAAACGAAGTGAACATAGAC
TATAGCAGTGATTTGGCTACTGTAATTATAGGTAAAGGTATAAAA
TAATGGAGCATTTTTTAGTTAAATGGAAATACTGTCAAGATTTAT
ACAAAATTTATTTCAGCAATTTTATTGAATAATTGTAATATACAA
AGCCCTGTGCTATTCTATGGGGGTCCTTAGATAAACTGAGTATAT
CGATCCCTGTATATAGCAAAATAATGTATTTTTAACTGGGTTGGA
GTATGAAAAATGTCAGAGAAGGATTCTATTGAATGTTTATCTTTC
ATGGAAAGTTCTTTCAGGGAAAGTTAAACAGAAAATAGTATGATA
TTTTTATTTACTTATAACTATAAAATTACATGCCATCTCATATAT
CTCACAACTATAAAATTATAAGAAAATAAAAATCTTTAAAATGAC
TTATCATGTGATACTTAGTGGTTTGCTAAAGTTTCTTTACGTGCA
CAACTATTTCTCAAACTGGTAATTGCATGCTTTTCTCAAGGCTCC
TTAAAAATGAGTCATTGGCTTTAGCTTTTTAGTTGACCAAATGCA
AACATCCTGAAGTAGAATGCAGGTCAAATATTTGCTTTTTGAGAA
GCATACTTCTATCAAATGCAAGTGGAATTCCTTTAAGCAGTCCTT
AGAAATTATCTCTAAAATTGTTGCCCTCATATTTTTTTGTCAGGA
CTAAGTTAAGTTGGAGTTAAAGTTAGCATTCCACCAGCTCTTTAT
AGCTTAGAGGTTTTTCTTAATTCTATGTTTTAAGAGTTTTAGTCA
ACATAGCTTATCTCTGTAATGTAAAGGACACTATTTTATGAGCAC
CGGTGCCCCAAACAGAATTCTTCCCTTAAATGATCAGCGTAACTC
CCTTTCCCTCCTCTATTTGATTTCTGGTAACATTAACTCACCAAA
GGTTCTCATGCATTTGTAATAAAACAGGAACAAGCTTCAAAGGAT
TTTAAACACTATTTAGTTGTGTGTTTCTTTGATGCAAAGAAGAAA
TACTGGATTGAACTGACTTTTCAAACATTACTTATAGAACTTTAA
ATAAAGGTACAAGGACACTTGAAAGGACTGCTCTATGAGGAGGTT
GAGTTTGTCAGTCTCTTTTGGTCACATCTCTATGATGAATAAAAA
TGAGTGTTAGGATGAACTTGTAGCCTCTGCCGACAAGGACTTGGC
TATGGACCATCTTGGTAAAGGCCATTTTTTTTTTTTTTATCCATA
GATGCCAGTTTCACATCTCACCACACTTTGACTATGGGTATAATG
AGGGCATAGCAGAGTAATTACCAATGAGGATCTTTACATATGCCA
TTCTTATTTTAAATAAGAAATATGATTTAATCATTGCTATCCTTA
TCTTTCTGGGGTTCTCAGTCTTAGACACGGACCTCTAAAAACTAA
CTGAGAAGTTTAGCAATGCTATGTAATACAAAACAACAGTCTTTT
TAAGAACTCTGACATCTAGAAGCTATCAACATCACAATAAAATTC
TCAACCAGAATAAAATGACTGAGTGGTTTATATCCACTCTTCAAC
ATTTAGGAATGTTGGGTCAGTAGAACTATATTTTCTTGAGGAGCT
CTAATACGTGGATAATAAATTTTTCAAGGCCTATCTATAGTCAAA
TATTTGTCACTATTTTGTCAAAACAGGACGTTCTTTTACCTCATC
TTACACCAGTACTCTGGTTAAGTGAAAACATTGTAAATGGCCAGA
CCAAATATGGGGAAATAAGTAAGATATTTTTTCTCTATCCAGTGG
TAGTTATAGAGCTTTGAAAATATTTTTGTTTTACATTGATGGATG
TTAATCCCTTTTATATATTAATAAAGTAAAAAGCTAAACAAACAG
AGCTAGGGAAAATATCGGAGTTGACTCTACTTATCTCCTTTAAGA
TAGCAGGCTTTTCTTTTACATGTCCATTGACTCTGGAAACTGGAC
TTCACAGTAAGGACCACCTTTTTGGTGTTAACCCTGTCTTAAGTA
GTTTCTTGCCAGGCACCGCCTCAGTTGTGAATCTCTGGAGAGATT
AGTTAAAAGGGTTACTGTTATTCAATTGATTAACCATGGTTGCTT
TTGTATTTTGGGGGGGGGGTTATAATAAAAATCTTGTCAGCACAA
CTTAATTTCCATCATTTGAAACATGTGTGATAAATATTCTACTGT
TTATACCACAGGACTCAAACTTTAAAAATTACAAATTGGATTTTA
AAGAAGCTACCAGCCTAGCTTGGAATGTGCACGTATTTTGAAATA
AATTGTTCTATTGTTGTTGCTAAAACCTTATTAAACAAATATTTA
AAATATTAATCAATTTTAAAAGTCCAATATTTTTATTTTATATTA
TTTTAATATGTGGAAAATTATACACAGAACATTGACTTTTCAAAA
ATTAAACAGTACATTGTGCAAAACCTTAATAAAAATTTCCCTATG
TCGTTTTTACTACATCCAATGTTTGTAAATTGCCAGAAAATAAAG
TAAGATGTCAATTACAGTAAAGGATTCTGGAATCATTTTGACTTA
ACTAAAGACCTCACAGTAGCCTGTGATATATAAATAAATCATTTC
TCTGGTTTGA

In some embodiments, an oligonucleotide comprises a region of complementarity to a PPP1R3A sequence as set forth in SEQ ID NO: 962 or SEQ ID NO: 963. In some embodiments, the oligonucleotide comprises a region of complementarity that is at least 80%, 85%, 90%, 95%, 96%, 97%, 98%, 99%, or 100% complementary to a PPP1R3A sequence as set forth in SEQ ID NO: 962 or SEQ ID NO: 963. In some embodiments, the oligonucleotide comprises a sequence that has at least 10, 11, 12, 13, 14, 15, 16, 17, 18, or 19 consecutive nucleotides that are perfectly complementary to a PPP1R3A sequence as set forth in SEQ ID NO: 962 or SEQ ID NO: 963. In some embodiments, an oligonucleotide may comprise a sequence that targets (e.g., is complementary to) an RNA version (i.e., wherein the T's are replaced with U's) of a PPP1R3A sequence as set forth in SEQ ID NO: 962 or SEQ ID NO: 963. In some embodiments, the oligonucleotide comprises a sequence that is complementary (e.g., at least 80%, 85%, 90%, 95%, 96%, 97%, 98%, 99%, or 100% complementary) to an RNA version of a PPP1R3A sequence as set forth in SEQ ID NO: 962 or SEQ ID NO: 963. In some embodiments, the oligonucleotide comprises a sequence that has at least 10, 11, 12, 13, 14, 15, 16, 17, 18, or 19 consecutive nucleotides that are perfectly complementary to an RNA version of a PPP1R3A sequence as set forth in SEQ ID NO: 962 or SEQ ID NO: 963.

In some embodiments, a PPP1R3A-targeting oligonucleotide comprises an antisense strand that comprises at least 10, 11, 12, 13, 14, 15, 16, 17, 18, or 19 consecutive nucleotides of a sequence comprising any one of SEQ ID NOs: 1012-1035. In some embodiments, a PPP1R3A-targeting oligonucleotide comprises an antisense strand that comprises any one of SEQ ID NOs: 1012-1035. In some embodiments, a PPP1R3A-targeting oligonucleotide comprises an antisense strand that comprises shares at least 70%, 75%, 80%, 85%, 90%, 95%, or 97% sequence identity with at least 12 or at least 15 consecutive nucleotides of any one of SEQ ID NOs: 1012-1035.

In some embodiments, a PPP1R3A-targeting oligonucleotide comprises an antisense strand that targets a PPP1R3A sequence comprising any one of SEQ ID NOs: 964-1011. In some embodiments, an oligonucleotide comprises an antisense strand comprising at least 10, 11, 12, 13, 14, 15, 16, 17, 18, or 19 nucleotides (e.g., consecutive nucleotides) that are complementary to a PPP1R3A sequence comprising any one of SEQ ID NOs: 964-1011. In some embodiments, a PPP1R3A-targeting oligonucleotide comprises an antisense strand comprising a sequence that is at least 70%, 75%, 80%, 85%, 90%, 95%, or 97% complementary with at least 12 or at least 15 consecutive nucleotides of any one of SEQ ID NOs: 964-1011.

In some embodiments, a PPP1R3A-targeting oligonucleotide comprises an antisense strand comprises a region of complementarity to a target sequence as set forth in any one of SEQ ID NOs: 964-1011. In some embodiments, the region of complementarity is at least 8, at least 9, at least 10, at least 11, at least 12, at least 13, at least 14, at least 15, at least 16, at least 17, or at least 19 nucleotides in length. In some embodiments, the region of complementarity is 8, 9, 10, 11, 12, 13, 14, 15, 16, 17, 18, or 19 nucleotides in length. In some embodiments, the region of complementarity is in the range of 8 to 20, 10 to 20 or 15 to 20 nucleotides in length. In some embodiments, the region of complementarity is fully complementary with all or a portion of its target sequence. In some embodiments, the region of complementarity includes 1, 2, 3 or more mismatches.

In some embodiments, a PPP1R3A-targeting oligonucleotide further comprises a sense strand that hybridizes to the antisense strand to form a double stranded siRNA. In some embodiments, the PPP1R3A-targeting oligonucleotide comprises an antisense strand that comprises the nucleotide sequence of any one of SEQ ID NOs: 1012-1035. In some embodiments, the PPP1R3A-targeting oligonucleotide further comprises a sense strand that comprises the nucleotide sequence of any one of SEQ ID NOs: 988-1011.

In some embodiments, the PPP1R3A-targeing oligonucleotide is a double stranded oligonucleotide (e.g., an siRNA) comprising an antisense strand that comprises the nucleotide sequence of any one of SEQ ID NOs: 1012-1035 and a sense strand that hybridizes to the antisense strand and comprises the nucleotide sequence of any one of SEQ ID NOs: 988-1011, wherein the antisense strand and/or (e.g., and) comprises one or more modified nucleosides (e.g., 2′-modified nucleosides). In some embodiment, the one or more modified nucleosides are selected from 2′-O-Me and 2′-F modified nucleosides.

In some embodiments, the PPP1R3A-targeing oligonucleotide is a double stranded oligonucleotide (e.g., an siRNA) comprising an antisense strand that comprises the nucleotide sequence of any one of SEQ ID NOs: 1012-1035 and a sense strand that hybridizes to the antisense strand and comprises the nucleotide sequence of any one of SEQ ID NOs: 988-1011, wherein the each nucleoside in the antisense strand and/or (e.g., and) each nucleoside in the sense strand is a 2′-modified nucleoside selected from 2′-O-Me and 2′-F modified nucleosides.

In some embodiments, the PPP1R3A-targeing oligonucleotide is a double stranded oligonucleotide (e.g., an siRNA) comprising an antisense strand that comprises the nucleotide sequence of any one of SEQ ID NOs: 1012-1035 and a sense strand that hybridizes to the antisense strand and comprises the nucleotide sequence of any one of SEQ ID NOs: 988-1011, wherein the each nucleoside in the antisense strand and each nucleoside in the sense strand is a 2′-modified nucleoside selected from 2′-O-Me and 2′-F modified nucleosides, and wherein the antisense strand and/or (e.g., and) the sense strand each comprises one or more phosphorothioate internucleoside linkages. In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages,

In some embodiments, the antisense strand of the PPP1R3A-targeing oligonucleotide comprises a structure of (5′ to 3′):
fNfNmNfNmNfNmNfNmNfNmNfNmNfNmNfNmNfNmNfNmN*fN*mN, wherein “mN” indicates 2′-O-methyl (2′-O-Me) modified nucleosides; “fN” indicates 2′-fluoro (2′-F) modified nucleosides; “*” indicates phosphrothioate internucleoside linkage; and the absence of “*” between two nucleosides indicate phosphodiester internucleoside linkage.

In some embodiments, the sense strand of the PPPR3A-targeing oligonucleotide comprises a structure of (5′ to 3′):
mNmNfNmNfNmNfNmNfNmNfNmNfNmNfNmNfNmNfNmNfN, wherein “inN” indicates 2′-O-methyl (2′-O-Me) modified nucleosides; “fN” indicates 2′-fluoro (2′-F) modified nucleosides; and the absence of “*” between two nucleosides indicate phosphodiester internucleoside linkage.

In some embodiments, the antisense strand of the PPP1R3A-targeing oligonucleotide is selected from the modified version of SEQ ID NOs: 1012-1035 listed in Table 40. In some embodiments, the sense strand of the PPP1R3A-targeing oligonucleotide is selected from the modified version of SEQ ID NOs: 988-1011 listed in Table 40. In some embodiments, the PPP1R3A-targeing oligonucleotide is a siRNA selected from the siRNAs listed in Table 40.

TABLE 38
PPP1R3A Target Sequences

| | Corre- | | |
|---|---|---|---|
| | sponding | | |
| | first | | |
| | nucleo- | | |
| | tide | | |
| | of Refer- | PPP1R3A Target | SEQ |
| Reference | ence | Sequence | ID |
| sequence | Sequence | (5′ to 3′) | NO: |
| NM_002711.4 (SEQ | 510 | GGCAGACACATTATGACAT | 964 |
| ID NO: 962) | | | |
| NM_002711.4 (SEQ | 3521 | TTTGCTCCTTCATATAAGT | 965 |
| ID NO: 962) | | | |
| NM_002711.4 (SEQ | 3535 | TAAGTAATTATTCTATATA | 966 |
| ID NO: 962) | | | |
| NM_002711.4 (SEQ | 3534 | ATAAGTAATTATTCTATAT | 967 |
| ID NO: 962) | | | |
| NM_002711.4 (SEQ | 3579 | TACCTATATGAATATGAGA | 968 |
| ID NO: 962) | | | |
| NM_002711.4 (SEQ | 3571 | TCATTAAATACCTATATGA | 969 |
| ID NO: 962) | | | |
| NM_002711.4 (SEQ | 2301 | ACAGGCCCATCGAGGTAAA | 970 |
| ID NO: 962) | | | |
| NM_002711.4 (SEQ | 517 | ACATTATGACATTTTAGCA | 971 |
| ID NO: 962) | | | |
| NM_002711.4 (SEQ | 508 | CTGGCAGACACATTATGAC | 972 |
| ID NO: 962) | | | |
| NM_002711.4 (SEQ | 626 | GTTGAGTTTTGTATACGTT | 973 |
| ID NO: 962) | | | |
| NM_002711.4 (SEQ | 3557 | CCATTATGTTTGGATCATT | 974 |
| ID NO: 962) | | | |
| NM_002711.4 (SEQ | 800 | GAAGAATCATCAGTAACAT | 975 |
| ID NO: 962) | | | |
| NM_002711.4 (SEQ | 745 | GAAAGAAGTTCCTAACAGA | 976 |
| ID NO: 962) | | | |
| NM_002711.4 (SEQ | 4155 | GTCAACTATTCAGAATTGA | 977 |
| ID NO: 962) | | | |
| NM_002711.4 (SEQ | 439 | CAAGGGTATTATTCGAGTT | 978 |
| ID NO: 962) | | | |
| NM_002711.4 (SEQ | 213 | CTGATTCCTTTGGATTCAA | 979 |
| ID NO: 962) | | | |
| NM_002711.4 (SEQ | 2382 | GAGATACAGTAGGTGTAAT | 980 |
| ID NO: 962) | | | |
| NM_080464.2 (SEQ | 3485 | CTTGGATCATTAAATACCT | 981 |
| ID NO: 963) | | | |
| NM_080464.2 (SEQ | 2264 | GACAGGCCCATCGAGGTAA | 982 |
| ID NO: 963) | | | |
| NM_080464.2 (SEQ | 3477 | CCATTATGCTTGGATCATT | 983 |
| ID NO: 963) | | | |
| NM_080464.2 (SEQ | 2461 | GGAGAAACATGGAATTGGA | 984 |
| ID NO: 963) | | | |
| NM_080464.2 (SEQ | 2367 | GGGAGTCACATTTAGATAT | 985 |
| ID NO: 963) | | | |
| NM_080464.2 (SEQ | 219 | GGAGAGTTTCCTTTGCTGA | 986 |
| ID NO: 963) | | | |
| NM_080464.2 (SEQ | 5173 | CTTAGAAATTATCTCTAAA | 987 |
| ID NO: 963) | | | |

* The target sequences contain Ts, but binding to RNA and/or DNA is contemplated.

In some embodiments, an oligonucleotide may comprise or consist of any sequence as provided in Table 39.

TABLE 39
Oligonucleotide sequences for targeting PPP1R3A

| Passenger Strand/ | | Guide Strand/ | |
|---|---|---|---|
| Sense Strand | SEQ | Antisense Strand | SEQ |
| (RNA) | ID | (RNA) | ID |
| (5′ to 3′) | NO: | (5′ to 3′) | NO: |
| CUGGCAGACACAUUAUGACAU | 988 | AUGUCAUAAUGUGUCUGCCAGUC | 1012 |
| AGUUUGCUCCUUCAUAUAAGU | 989 | ACUUAUAUGAAGGAGCAAACUUA | 1013 |
| UAUAAGUAAUUAUUCUAUAUA | 990 | UAUAUAGAAUAAUUACUUAUAUG | 1014 |
| AUAUAAGUAAUUAUUCUAUAU | 991 | AUAUAGAAUAAUUACUUAUAUGA | 1015 |
| AAUACCUAUAUGAAUAUGAGA | 992 | UCUCAUAUUCAUAUAGGUAUUUA | 1016 |
| GAUCAUUAAAUACCUAUAUGA | 993 | UCAUAUAGGUAUUUAAUGAUCCA | 1017 |
| UGACAGGCCCAUCGAGGUAAA | 994 | UUUACCUCGAUGGGCCUGUCACU | 1018 |
| ACACAUUAUGACAUUUUAGCA | 995 | UGCUAAAAUGUCAUAAUGUGUCU | 1019 |
| GACUGGCAGACACAUUAUGAC | 996 | GUCAUAAUGUGUCUGCCAGUCAU | 1020 |
| AAGUUGAGUUUUGUAUACGUU | 997 | AACGUAUACAAAACUCAACUUUA | 1021 |
| GACCAUUAUGUUUGGAUCAUU | 998 | AAUGAUCCAAACAUAAUGGUCCU | 1022 |
| AAGAAGAAUCAUCAGUAACAU | 999 | AUGUUACUGAUGAUUCUUCUUUA | 1023 |
| UGGAAAGAAGUUCCUAACAGA | 1000 | UCUGUUAGGAACUUCUUUCCAUG | 1024 |
| GUGUCAACUAUUCAGAAUUGA | 1001 | UCAAUUCUGAAUAGUUGACACUG | 1025 |
| AUCAAGGGUAUUAUUCGAGUU | 1002 | AACUCGAAUAAUACCCUUGAUAC | 1026 |
| UGCUGAUUCCUUUGGAUUCAA | 1003 | UUGAAUCCAAAGGAAUCAGCAAA | 1027 |
| ACGAGAUACAGUAGGUGUAAU | 1004 | AUUACACCUACUGUAUCUCGUUG | 1028 |
| UGCUUGGAUCAUUAAAUACCU | 1005 | AGGUAUUUAAUGAUCCAAGCAUA | 1029 |
| AUGACAGGCCCAUCGAGGUAA | 1006 | UUACCUCGAUGGGCCUGUCAUUU | 1030 |
| GACCAUUAUGCUUGGAUCAUU | 1007 | AAUGAUCCAAGCAUAAUGGUCCU | 1031 |
| AAGGAGAAACAUGGAAUUGGA | 1008 | UCCAAUUCCAUGUUUCUCCUUGU | 1032 |
| AAGGGAGUCACAUUUAGAUAU | 1009 | AUAUCUAAAUGUGACUCCCUUUC | 1033 |
| CCGGAGAGUUUCCUUUGCUGA | 1010 | UCAGCAAAGGAAACUCUCCGGGA | 1034 |
| UCCUUAGAAAUUAUCUCUAAA | 1011 | UUUAGAGAUAAUUUCUAAGGACU | 1035 |

In some embodiments, an oligonucleotide is a modified oligonucleotide as provided in Table 40, wherein ‘mN’ represents a 2′-O-methyl modified nucleoside (e.g., mU is 2′-O-methyl modified uridine), ‘fi’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 40
Modified Oligonucleotides for targeting PPP1R3A

| | Modified Passenger | | Modified Guide | |
|---|---|---|---|---|
| | Strand/Sense | SEQ | Strand/Antisense | SEQ |
| | Strand (RNA) | ID | Strand (RNA) | ID |
| siRNA # | (5′ to 3′) | NO: | (5′ to 3′) | NO: |
| hsPPP1R3A-1 | mCmUfGmGfCmAfGmAfC | 988 | fAfUmGfUmCfAmUfAmAfUm | 1012 |
| | mAfCmAfUmUfAmUfGmA | | GfUmGfUmCfUmGfCmCfAmG | |
| | fCmAfU | | *fU*mC | |
| hsPPP1R3A-2 | mAmGfUmUfUmGfCmUfC | 989 | fAfCmUfUmAfUmAfUmGfAm | 1013 |
| | mCfUmUfCmAfUmAfUmAf | | AfGmGfAmGfCmAfAmAfCmU | |
| | AmGfU | | *fU*mA | |
| hsPPP1R3A-3 | mUmAfUmAfAmGfUmAfA | 990 | fUfAmUfAmUfAmGfAmAfUm | 1014 |
| | mUfUmAfUmUfCmUfAmU | | AfAmUfUmAfCmUfUmAfUmA | |
| | fAmUfA | | *fU*mG | |
| hsPPP1R3A-4 | mAmUfAmUfAmAfGmUfA | 991 | fAfUmAfUmAfGmAfAmUfAm | 1015 |
| | mAfUmUfAmUfUmCfUmA | | AfUmUfAmCfUmUfAmUfAmU | |
| | fUmAfU | | *fG*mA | |
| hsPPP1R3A-5 | mAmAfUmAfCmCfUmAfU | 992 | fUfCmUfCmAfUmAfUmUfCmA | 1016 |
| | mAfUmGfAmAfUmAfUmG | | fUmAfUmAfGmGfUmAfUmU*f | |
| | fAmGfA | | U*mA | |
| hsPPP1R3A-6 | mGmAfUmCfAmUfUmAfA | 993 | fUfCmAfUmAfUmAfGmGfUm | 1017 |
| | mAfUmAfCmCfUmAfUmAf | | AfUmUfUmAfAmUfGmAfUmC | |
| | UmGfA | | *fC*mA | |
| hsPPP1R3A-7 | mUmGfAmCfAmGfGmCfC | 994 | fUfUmUfAmCfCmUfCmGfAmU | 1018 |
| | mCfAmUfCmGfAmGfGmUf | | fGmGfGmCfCmUfGmUfCmA*f | |
| | AmAfA | | C*mU | |
| hsPPP1R3A-8 | mAmCfAmCfAmUfUmAfU | 995 | fUfGmCfUmAfAmAfAmUfGm | 1019 |
| | mGfAmCfAmUfUmUfUmA | | UfCmAfUmAfAmUfGmUfGmU | |
| | fGmCfA | | *fC*mU | |
| hsPPP1R3A-9 | mGmAfCmUfGmGfCmAfG | 996 | fGfUmCfAmUfAmAfUmGfUm | 1020 |
| | mAfCmAfCmAfUmUfAmUf | | GfUmCfUmGfCmCfAmGfUmC | |
| | GmAfC | | *fA*mU | |
| hsPPP1R3A- | mAmAfGmUfUmGfAmGfU | 997 | fAfAmCfGmUfAmUfAmCfAmA | 1021 |
| 10 | mUfUmUfGmUfAmUfAmC | | fAmAfCmUfCmAfAmCfUmU*f | |
| | fGmUfU | | U*mA | |
| hsPPP1R3A- | mGmAfCmCfAmUfUmAfU | 998 | fAfAmUfGmAfUmCfCmAfAmA | 1022 |
| 11 | mGfUmUfUmGfGmAfUmC | | fCmAfUmAfAmUfGmGfUmC*f | |
| | fAmUfU | | C*mU | |
| hsPPP1R3A- | mAmAfGmAfAmGfAmAfU | 999 | fAfUmGfUmUfAmCfUmGfAm | 1023 |
| 12 | mCfAmUfCmAfGmUfAmAf | | UfGmAfUmUfCmUfUmCfUmU | |
| | CmAfU | | *fU*mA | |
| hsPPP1R3A- | mUmGfGmAfAmAfGmAfA | 1000 | fUfCmUfGmUfUmAfGmGfAm | 1024 |
| 13 | mGfUmUfCmCfUmAfAmCf | | AfCmUfUmCfUmUfUmCfCmA | |
| | AmGfA | | *fU*mG | |
| hsPPP1R3A- | mGmUfGmUfCmAfAmCfU | 1001 | fUfCmAfAmUfUmCfUmGfAmA | 1025 |
| 14 | mAfUmUfCmAfGmAfAmU | | fUmAfGmUfUmGfAmCfAmC*f | |
| | fUmGfA | | U*mG | |
| hsPPP1R3A- | mAmUfCmAfAmGfGmGfU | 1002 | fAfAmCfUmCfGmAfAmUfAmA | 1026 |
| 15 | mAfUmUfAmUfUmCfGmA | | fUmAfCmCfCmUfUmGfAmU*f | |
| | fGmUfU | | A*mC | |
| hsPPP1R3A- | mUmGfCmUfGmAfUmUfC | 1003 | fUfUmGfAmAfUmCfCmAfAmA | 1027 |
| 16 | mCfUmUfUmGfGmAfUmU | | fGmGfAmAfUmCfAmGfCmA*f | |
| | fCmAfA | | A*mA | |
| hsPPP1R3A- | mAmCfGmAfGmAfUmAfC | 1004 | fAfUmUfAmCfAmCfCmUfAmC | 1028 |
| 17 | mAfGmUfAmGfGmUfGmU | | fUmGfUmAfUmCfUmCfGmU*f | |
| | fAmAfU | | U*mG | |
| mmPPP1R3A- | mUmGfCmUfUmGfGmAfU | 1005 | fAfGmGfUmAfUmUfUmAfAm | 1029 |
| 1 | mCfAmUfUmAfAmAfUmA | | UfGmAfUmCfCmAfAmGfCmA | |
| | fCmCfU | | *fU*mA | |
| mmPPP1R3A- | mAmUfGmAfCmAfGmGfC | 1006 | fUfUmAfCmCfUmCfGmAfUmG | 1030 |
| 2 | mCfCmAfUmCfGmAfGmGf | | fGmGfCmCfUmGfUmCfAmU*f | |
| | UmAfA | | U*mU | |
| mmPPP1R3A- | mGmAfCmCfAmUfUmAfU | 1007 | fAfAmUfGmAfUmCfCmAfAmG | 1031 |
| 3 | mGfCmUfUmGfGmAfUmCf | | fCmAfUmAfAmUfGmGfUmC*f | |
| | AmUfU | | C*mU | |
| mmPPP1R3A- | mAmAfGmGfAmGfAmAfA | 1008 | fUfCmCfAmAfUmUfCmCfAmU | 1032 |
| 4 | mCfAmUfGmGfAmAfUmU | | fGmUfUmUfCmUfCmCfUmU*f | |
| | fGmGfA | | G*mU | |
| mmPPP1R3A- | mAmAfGmGfGmAfGmUfC | 1009 | fAfUmAfUmCfUmAfAmAfUm | 1033 |
| 5 | mAfCmAfUmUfUmAfGmA | | GfUmGfAmCfUmCfCmCfUmU | |
| | fUmAfU | | *fU*mC | |
| mmPPP1R3A- | mCmCfGmGfAmGfAmGfU | 1010 | fUfCmAfGmCfAmAfAmGfGmA | 1034 |
| 6 | mUfUmCfCmUfUmUfGmCf | | fAmAfCmUfCmUfCmCfGmG*f | |
| | UmGfA | | G*mA | |
| mmPPP1R3A- | mUmCfCmUfUmAfGmAfA | 1011 | fUfUmUfAmGfAmGfAmUfAm | 1035 |
| 7 | mAfUmUfAmUfCmUfCmUf | | AfUmUfUmCfUmAfAmGfGmA | |
| | AmAfA | | *fC*mU | |

In some embodiments, any one of the MSTN targeting oligonucleotides, INHBA targeting oligonucleotides, ACVR1B targeting oligonucleotides, MLCK1 targeting oligonucleotides, ACVR1 targeting oligonucleotides, FBXO32 oligonucleotides, TRIM63 oligonucleotides, MEF21D targeting oligonucleotides, KLF15 targeting oligonucleotides, MED1 targeting oligonucleotides, MED13 targeting oligonucleotides, or PPP1R3A targeting oligonucleotides can be in salt form, e.g., as sodium, potassium, or magnesium salts.

In some embodiments, the 5′ or 3′ nucleoside (e.g., terminal nucleoside) of any one of the oligonucleotides described herein (e.g., the oligonucleotides listed in Tables 9, 10, 12, 13, 15, 16, 18, 19, 21, 22, 24, 25, 27, 28, 30, 21, 33, 34, 36, 37, 39, and 40) is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein (e.g., the oligonucleotides listed in Tables 9, 10, 12, 13, 15, 16, 18, 19, 21, 22, 24, 25, 27, 28, 30, 31, 33, 34, 36, 37, 39, and 40) is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein (e.g., the oligonucleotides listed in Tables 9, 10, 12, 13, 15, 16, 18, 19, 21, 22, 24, 25, 27, 28, 30, 31, 33, 34, 36, 37, 39, and 40) is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via the amine group.

m. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., an mRNA or pre-mRNA molecule) results in modulation of activity or expression of the target (e.g., decreased mRNA translation, altered pre-mRNA splicing, exon skipping, target mRNA degradation, etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide comprises an antisense strand that is complementary (e.g., at least 85% at least 90%, at least 95%, or 100%) to a target sequence of any one of the antisense strands provided herein (e.g., the antisense strands listed in Tables 9, 10, 12, 13, 15, and 16). In some embodiments, the oligonucleotide comprises an antisense strand that is complementary (e.g., at least 85% at least 90%, at least 95%, or 100%) to a target sequence of any one of the antisense strands provided herein (e.g., the antisense strands listed in Tables 18, 19, 21, 22, 24, 25, 27, 28, 30, 31, 33, 34, 36, 37, 39, and 40). In some embodiments, such target sequence is 100% complementary to the oligonucleotide listed in Tables 9, 10, 12, 13, 15, and 16. In some embodiments, such target sequence is 100% complementary to the oligonucleotide listed in Tables 18, 19, 21, 22, 24, 25, 27, 28, 30, 31, 33, 34, 36, 37, 39, and 40. In some embodiments, the oligonucleotide is an siRNA molecule comprising an antisense strand comprising a nucleotide sequence that is complementary (e.g., at least 85%, at least 90%, at least 95%, or 100%) to the target RNA sequence of the oligonucleotides provided herein (e.g., in Tables 8, 11, and 14). In some embodiments, the oligonucleotide is an siRNA molecule comprising an antisense strand comprising a nucleotide sequence that is complementary (e.g., at least 85%, at least 90%, at least 95%, or 100%) to the target RNA sequence of the oligonucleotides provided herein (e.g., in Tables 17, 20, 23, 26, 29, 32, 35, and 38).

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in Tables 9, 10, 12, 13, 15, and 16) may optionally be uracil bases (U's), and/or any one or more of the U's may optionally be T's. In some embodiments, any one or more of the thymine bases (T's) in any one of the oligonucleotides provided herein (e.g., the oligonucleotides listed in Tables 18, 19, 21, 22, 24, 25, 27, 28, 30, 31, 33, 34, 36, 37, 39, and 40) may optionally be uracil bases (U's), and/or any one or more of the U's may optionally be T's.

n. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

o. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

p. Internucleotide Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

q. Stereospecific Oligonucleotides

In some embodiments, internucleotidic phosphorus atoms of oligonucleotides are chiral, and the properties of the oligonucleotides are adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides are provided that comprise nucleoside units that are joined together by either substantially all Sp or substantially all Rp phosphorothioate intersugar linkages. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

r. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

s. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publications that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

t. Gapmers

In some embodiments, an oligonucleotide described herein is a gapmer. A gapmer oligonucleotide generally has the formula 5′-X—Y—Z-3′, with X and Z as flanking regions around a gap region Y. In some embodiments, flanking region X of formula 5′-X—Y—Z-3′ is also referred to as X region, flanking sequence X, 5′ wing region X, or 5′ wing segment. In some embodiments, flanking region Z of formula 5′-X—Y—Z-3′ is also referred to as Z region, flanking sequence Z, 3′ wing region Z, or 3′ wing segment. In some embodiments, gap region Y of formula 5′-X—Y—Z-3′ is also referred to as Y region, Y segment, or gap-segment Y. In some embodiments, each nucleoside in the gap region Y is a 2′-deoxyribonucleoside, and neither the 5′ wing region X or the 3′ wing region Z contains any 2′-deoxyribonucleosides.

In some embodiments, the Y region is a contiguous stretch of nucleotides, e.g., a region of 6 or more DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleosides, e.g., one to six high-affinity modified nucleosides. Examples of high affinity modified nucleosides include, but are not limited to, 2′-modified nucleosides (e.g., 2′-MOE, 2′O-Me, 2′-F) or 2′-4′ bicyclic nucleosides (e.g., LNA, cEt, ENA). In some embodiments, the flanking sequences X and Z may be of 1-20 nucleotides, 1-8 nucleotides, or 1-5 nucleotides in length. The flanking sequences X and Z may be of similar length or of dissimilar lengths. In some embodiments, the gap-segment Y may be a nucleotide sequence of 5-20 nucleotides, 5-15 twelve nucleotides, or 6-10 nucleotides in length.

In some embodiments, the gap region of the gapmer oligonucleotides may contain modified nucleotides or nucleosides known to be acceptable for efficient RNase H action in addition to DNA nucleosides, such as C4′-substituted nucleosides, acyclic nucleosides, and arabino-configured nucleosides. In some embodiments, the gap region comprises one or more unmodified internucleoside linkages. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,015,315; 7,101,993; 7,399,845; 7,432,250; 7,569,686; 7,683,036; 7,750,131; 8,580,756; 9,045,754; 9,428,534; 9,695,418; 10,017,764; 10,260,069; 9,428,534; 8,580,756; U.S. patent publication Nos. US20050074801, US20090221685; US20090286969, US20100197762, and US20110112170; PCT publication Nos. WO2004069991; WO2005023825; WO2008049085 and WO2009090182; and EP Patent No. EP2,149,605, each of which is herein incorporated by reference in its entirety.

In some embodiments, a gapmer is 10-40 nucleosides in length. For example, a gapmer may be 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length.

In some embodiments, the gap region Y in a gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosines.

In some embodiments, the 5′wing region of a gapmer (X in the 5′-X—Y—Z-3′ formula) and the 3′wing region of a gapmer (Z in the 5′-X—Y—Z-3′ formula) are independently 1-20 nucleosides long. For example, the 5′wing region of a gapmer (X in the 5′-X—Y—Z-3′ formula) and the 3′wing region of the gapmer (Z in the 5′-X—Y—Z-3′ formula) may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′wing region of the gapmer (X in the 5′-X—Y—Z-3′ formula) and the 3′wing region of the gapmer (Z in the 5′-X—Y—Z-3′ formula) are independently 1, 2, 3, 4, 5, 6, 7, 8, 9, 10, 11, 12, 13, 14, 15, 16, 17, 18, 19, or 20 nucleosides long. In some embodiments, the 5′wing region of the gapmer (X in the 5′-X—Y—Z-3′ formula) and the 3′wing region of the gapmer (Z in the 5′-X—Y—Z-3′ formula) are of the same length. In some embodiments, the 5′wing region of the gapmer (X in the 5′-X—Y—Z-3′ formula) and the 3′wing region of the gapmer (Z in the 5′-X—Y—Z-3′ formula) are of different lengths. In some embodiments, the 5′wing region of the gapmer (X in the 5′-X—Y—Z-3′ formula) is longer than the 3′wing region of the gapmer (Z in the 5′-X—Y—Z-3′ formula). In some embodiments, the 5′wing region of the gapmer (X in the 5′-X—Y—Z-3′ formula) is shorter than the 3′wing region of the gapmer (Z in the 5′-X—Y—Z-3′ formula).

In some embodiments, a gapmer comprises a 5′-X—Y—Z-3′ of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1,2-14-2, 1-13-4, 4-13-1, 2-13-3,3-13-2, 1-12-5, 5-12-1, 2-12-4, 4-12-2,3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3,3-14-2, 1-13-5, 5-13-1, 2-13-4, 4-13-2,3-13-3, 1-12-6, 6-12-1, 2-12-5, 5-12-2,3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3,3-15-2, 1-14-5, 5-14-1, 2-14-4, 4-14-2,3-14-3, 1-13-6, 6-13-1, 2-13-5, 5-13-2,3-13-4, 4-13-3, 1-12-7,7-12-1, 2-12-6, 6-12-2,3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3,3-15-2, 1-14-5, 2-14-4, 4-14-2,3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3,3-16-2, 1-15-5, 2-15-4, 4-15-2,3-15-3, 1-14-6, 6-14-1, 2-14-5, 5-14-2,3-14-4, 4-14-3, 1-13-7,7-13-1, 2-13-6, 6-13-2,3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12-3, 4-12-7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions in the 5′-X—Y—Z-3′ gapmer.

In some embodiments, one or more nucleosides in the 5′wing region of a gapmer (X in the 5′-X—Y—Z-3′ formula) or the 3′wing region of a gapmer (Z in the 5′-X—Y—Z-3′ formula) are modified nucleosides (e.g., high-affinity modified nucleosides). In some embodiments, the modified nucleoside (e.g., high-affinity modified nucleosides) is a 2′-modifeid nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-0-dimethylaminoethyloxyethyl (2′-O-DMAEOE), or 2′-O—N-methylacetamido (2′-O-NMA)).

In some embodiments, one or more nucleosides in the 5′wing region of a gapmer (X in the 5′-X—Y—Z-3′ formula) are high-affinity modified nucleosides. In some embodiments, each nucleoside in the 5′wing region of the gapmer (X in the 5′-X—Y—Z-3′ formula) is a high-affinity modified nucleoside. In some embodiments, one or more nucleosides in the 3′wing region of a gapmer (Z in the 5′-X—Y—Z-3′ formula) are high-affinity modified nucleosides. In some embodiments, each nucleoside in the 3′wing region of the gapmer (Z in the 5′-X—Y—Z-3′ formula) is a high-affinity modified nucleoside. In some embodiments, one or more nucleosides in the 5′wing region of the gapmer (X in the 5′-X—Y—Z-3′ formula) are high-affinity modified nucleosides and one or more nucleosides in the 3′wing region of the gapmer (Z in the 5′-X—Y—Z-3′ formula) are high-affinity modified nucleosides. In some embodiments, each nucleoside in the 5′wing region of the gapmer (X in the 5′-X—Y—Z-3′ formula) is a high-affinity modified nucleoside and each nucleoside in the 3′wing region of the gapmer (Z in the 5′-X—Y—Z-3′ formula) is high-affinity modified nucleoside.

In some embodiments, the 5′wing region of a gapmer (X in the 5′-X—Y—Z-3′ formula) comprises the same high affinity nucleosides as the 3′wing region of the gapmer (Z in the 5′-X—Y—Z-3′ formula). For example, the 5′wing region of the gapmer (X in the 5′-X—Y—Z-3′ formula) and the 3′wing region of the gapmer (Z in the 5′-X—Y—Z-3′ formula) may comprise one or more non-bicyclic 2′-modified nucleosides (e.g., 2′-MOE or 2′-O-Me). In another example, the 5′wing region of the gapmer (X in the 5′-X—Y—Z-3′ formula) and the 3′wing region of the gapmer (Z in the 5′-X—Y—Z-3′ formula) may comprise one or more 2′-4′ bicyclic nucleosides (e.g., LNA or cEt). In some embodiments, each nucleoside in the 5′wing region of the gapmer (X in the 5′-X—Y—Z-3′ formula) and the 3′wing region of the gapmer (Z in the 5′-X—Y—Z-3′ formula) is a non-bicyclic 2′-modified nucleosides (e.g., 2′-MOE or 2′-O-Me). In some embodiments, each nucleoside in the 5′wing region of the gapmer (X in the 5′-X—Y—Z-3′ formula) and the 3′wing region of the gapmer (Z in the 5′-X—Y—Z-3′ formula) is a 2′-4′ bicyclic nucleosides (e.g., LNA or cEt).

In some embodiments, a gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a non-bicyclic 2′-modified nucleosides (e.g., 2′-MOE or 2′-O-Me) and each nucleoside in Y is a 2′-deoxyribonucleoside. In some embodiments, the gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a 2′-4′ bicyclic nucleosides (e.g., LNA or cEt) and each nucleoside in Y is a 2′-deoxyribonucleoside. In some embodiments, the 5′wing region of the gapmer (X in the 5′-X—Y—Z-3′ formula) comprises different high affinity nucleosides as the 3′wing region of the gapmer (Z in the 5′-X—Y—Z-3′ formula). For example, the 5′wing region of the gapmer (X in the 5′-X—Y—Z-3′ formula) may comprise one or more non-bicyclic 2′-modified nucleosides (e.g., 2′-MOE or 2′-O-Me) and the 3′wing region of the gapmer (Z in the 5′-X—Y—Z-3′ formula) may comprise one or more 2′-4′ bicyclic nucleosides (e.g., LNA or cEt). In another example, the 3′wing region of the gapmer (Z in the 5′-X—Y—Z-3′ formula) may comprise one or more non-bicyclic 2′-modified nucleosides (e.g., 2′-MOE or 2′-O-Me) and the 5′wing region of the gapmer (X in the 5′-X—Y—Z-3′ formula) may comprise one or more 2′-4′ bicyclic nucleosides (e.g., LNA or cEt).

In some embodiments, a gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a non-bicyclic 2′-modified nucleosides (e.g., 2′-MOE or 2′-O-Me), each nucleoside in Z is a 2′-4′ bicyclic nucleosides (e.g., LNA or cEt), and each nucleoside in Y is a 2′-deoxyribonucleoside. In some embodiments, the gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a 2′-4′ bicyclic nucleosides (e.g., LNA or cEt), each nucleoside in Z is a non-bicyclic 2′-modified nucleosides (e.g., 2′-MOE or 2′-O-Me) and each nucleoside in Y is a 2′-deoxyribonucleoside.

In some embodiments, the 5′wing region of a gapmer (X in the 5′-X—Y—Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, the 3′wing region of the gapmer (Z in the 5′-X—Y—Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, both the 5′wing region of the gapmer (X in the 5′-X—Y—Z-3′ formula) and the 3′wing region of the gapmer (Z in the 5′-X—Y—Z-3′ formula) comprise one or more non-bicyclic 2′-modified nucleosides (e.g., 2′-MOE or 2′-O-Me) and one or more 2′-4′ bicyclic nucleosides (e.g., LNA or cEt).

In some embodiments, a gapmer comprises a 5′-X—Y—Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X—Y—Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′—X—Y—Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

Non-limiting examples of gapmers configurations with a mix of non-bicyclic 2′-modified nucleoside (e.g., 2′-MOE or 2′-O-Me) and 2′-4′ bicyclic nucleosides (e.g., LNA or cEt) in the 5′wing region of the gapmer (X in the 5′-X—Y—Z-3′ formula) and/or the 3′wing region of the gapmer (Z in the 5′-X—Y—Z-3′ formula) include: BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BABA-(D)n-ABAB; KAKA-(D)n-AKAK; LALA-(D)n-ALAL; BEBE-(D)n-EBEB; KEKE-(D)n-EKEK; LELE-(D)n-ELEL; BABA-(D)n-ABAB; KAKA-(D)n-AKAK; LALA-(D)n-ALAL; BEBE-(D)n-EBEB; KEKE-(D)n-EKEK; LELE-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; AABB-(D)n-BBAA; BBAA-(D)n-AABB; AAKK-(D)n-KKAA; AALL-(D)n-LLAA; EEBB-(D)n-BBEE; EEKK-(D)n-KKEE; EELL-(D)n-LLEE; AABB-(D)n-BBAA; AAKK-(D)n-KKAA; AALL-(D)n-LLAA; EEBB-(D)n-BBEE; EEKK-(D)n-KKEE; EELL-(D)n-LLEE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; ABBAABB-(D)n-BB; AKKAAKK-(D)n-KK; ALLAALLL-(D)n-LL; EBBEEBB-(D)n-BB; EKKEEKK-(D)n-KK; ELLEELL-(D)n-LL; ABBAABB-(D)n-BB; AKKAAKK-(D)n-KK; ALLAALL-(D)n-LL; EBBEEBB-(D)n-BB; EKKEEKK-(D)n-KK; ELLEELL-(D)n-LL; ABBABB-(D)n-BBB; AKKAKK-(D)n-KKK; ALLALLL-(D)n-LLL; EBBEBB-(D)n-BBB; EKKEKK-(D)n-KKK; ELLELL-(D)n-LLL; ABBABB-(D)n-BBB; AKKAKK-(D)n-KKK; ALLALL-(D)n-LLL; EBBEBB-(D)n-BBB; EKKEKK-(D)n-KKK; ELLELL-(D)n-LLL; EEEK-(D)n-EEEEEEEE; EEK-(D)n-EEEEEEEEE; EK-(D)n-EEEEEEEEEE; EK-(D)n-EEEKK; K-(D)n-EEEKEKE; K-(D)n-EEEKEKEE; K-(D)n-EEKEK; EK-(D)n-EEEEKEKE; EK-(D)n-EEEKEK; EEK-(D)n-KEEKE; EK-(D)n-EEKEK; EK-(D)n-KEEK; EEK-(D)n-EEEKEK; EK-(D)n-KEEEKEE; EK-(D)n-EEKEKE; EK-(D)n-EEEKEKE; and EK-(D)n-EEEEKEK;. “A” nucleosides comprise a 2′-modified nucleoside; “B” represents a 2′-4′ bicyclic nucleoside; “K” represents a constrained ethyl nucleoside (cEt); “L” represents an LNA nucleoside; and “E” represents a 2′-MOE modified ribonucleoside; “D” represents a 2′-deoxyribonucleoside; “n” represents the length of the gap segment (Y in the 5′-X—Y—Z-3′ configuration) and is an integer between 1-20.

In some embodiments, any one of the gapmers described herein comprises one or more modified nucleoside linkages (e.g., a phosphorothioate linkage) in each of the X, Y, and Z regions. In some embodiments, each internucleoside linkage in the any one of the gapmers described herein is a phosphorothioate linkage. In some embodiments, each of the X, Y, and Z regions independently comprises a mix of phosphorothioate linkages and phosphodiester linkages. In some embodiments, each internucleoside linkage in the gap region Y is a phosphorothioate linkage, the 5′wing region X comprises a mix of phosphorothioate linkages and phosphodiester linkages, and the 3′wing region Z comprises a mix of phosphorothioate linkages and phosphodiester linkages.

u. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g., at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleoside, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

v. RNA Interference (RNAi)

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective. In some embodiments, the siRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the siRNA molecules are 8 to 30 base pairs in length, 10 to 15 base pairs in length, 10 to 20 base pairs in length, 15 to 25 base pairs in length, 19 to 21 base pairs in length, 21 to 23 base pairs in length.

Following selection of an appropriate target RNA sequence, siRNA molecules that comprise a nucleotide sequence complementary to all or a portion of the target sequence, i.e., an antisense sequence, can be designed and prepared using appropriate methods (see, e.g., PCT Publication Number WO 2004/016735; and U.S. Patent Publication Nos. 2004/0077574 and 2008/0081791). The siRNA molecule can be double stranded (i.e., a dsRNA molecule comprising an antisense strand and a complementary sense strand that hybridizes to form the dsRNA) or single-stranded (i.e., a ssRNA molecule comprising just an antisense strand). The siRNA molecules can comprise a duplex, asymmetric duplex, hairpin or asymmetric hairpin secondary structure, having self-complementary sense and antisense strands.

In some embodiments, the antisense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths.

In some embodiments, the sense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths.

In some embodiments, siRNA molecules comprise an antisense strand comprising a region of complementarity to a target region in a target mRNA. In some embodiments, the region of complementarity is at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region is a region of consecutive nucleotides in the target mRNA. In some embodiments, a complementary nucleotide sequence need not be 100% complementary to that of its target to be specifically hybridizable or specific for a target RNA sequence.

In some embodiments, siRNA molecules comprise an antisense strand that comprises a region of complementarity to a target RNA sequence and the region of complementarity is in the range of 8 to 15, 8 to 30, 8 to 40, or 10 to 50, or 5 to 50, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of a target RNA sequence. In some embodiments, siRNA molecules comprise a nucleotide sequence that contains no more than 1, 2, 3, 4, or 5 base mismatches compared to the portion of the consecutive nucleotides of target RNA sequence. In some embodiments, siRNA molecules comprise a nucleotide sequence that has up to 3 mismatches over 15 bases, or up to 2 mismatches over 10 bases.

In some embodiments, siRNA molecules comprise an antisense strand comprising a nucleotide sequence that is complementary (e.g., at least 85%, at least 90%, at least 95%, or 100%) to the target RNA sequence of the oligonucleotides provided herein (e.g., in Tables 8, 11, and 14). In some embodiments, siRNA molecules comprise an antisense strand comprising a nucleotide sequence that is complementary (e.g., at least 85%, at least 90%, at least 95%, or 100%) to the target RNA sequence of the oligonucleotides provided herein (e.g., in Tables 17, 20, 23, 26, 29, 32, 35, and 38). In some embodiments, siRNA molecules comprise an antisense strand comprising a nucleotide sequence that is at least 85%, at least 90%, at least 95%, or 100% identical to the oligonucleotides provided herein (e.g., in Tables 9, 10, 12, 13, 15, and 16). In some embodiments, siRNA molecules comprise an antisense strand comprising a nucleotide sequence that is at least 85%, at least 90%, at least 95%, or 100% identical to the oligonucleotides provided herein (e.g., in Tables 18, 19, 21, 22, 24, 25, 27, 28, 30, 31, 33, 34, 36, 37, 39, and 40). In some embodiments, siRNA molecules comprise an antisense strand comprising at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of the oligonucleotides provided herein (e.g., in Tables 9, 10, 12, 13, 15, and 16). In some embodiments, siRNA molecules comprise an antisense strand comprising at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of the oligonucleotides provided herein (e.g., in Tables 18, 19, 21, 22, 24, 25, 27, 28, 30, 31, 33, 34, 36, 37, 39, and 40). In some embodiments, siRNA molecules comprise an antisense strand comprising a nucleotide sequence that is complementary (e.g., at least 85%, at least 90%, at least 95%, or 100%) to the MLCK1 mRNA sequence as set forth in SEQ ID NO: 411.

Double-stranded siRNA may comprise sense and anti-sense RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or (e.g., and) the 5′ end of either or both strands). A spacer sequence may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule. In some embodiments, the siRNA molecule comprises 3′ overhangs of about 1 to about 3 nucleotides on the sense strand. In some embodiments, the siRNA molecule comprises 3′ overhangs of about 1 to about 3 nucleotides on the antisense strand. In some embodiments, the siRNA molecule comprises 3′ overhangs of about 1 to about 3 nucleotides on both the sense strand and the antisense strand.

In some embodiments, the siRNA molecule comprises one or more modified nucleotides or nucleosides (e.g., 1, 2, 3, 4, 5, 6, 7, 8, 9, 10 or more). In some embodiments, the siRNA molecule comprises one or more modified nucleotides/nucleosides and/or (e.g., and) one or more modified internucleotide linkages. In some embodiments, the modified nucleoside comprises a modified sugar moiety (e.g., a 2′ modified nucleoside). In some embodiments, the siRNA molecule comprises one or more 2′ modified nucleotides or nucleosides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, each nucleoside of the siRNA molecule is a modified nucleoside (e.g., a 2′-modified nucleoside). In some embodiments, the siRNA molecule comprises one or more phosphorodiamidate morpholinos. In some embodiments, each nucleotide of the siRNA molecule is a phosphorodiamidate morpholino.

In some embodiments, the siRNA molecule contains a phosphorothioate or other modified internucleoside linkage. In some embodiments, the siRNA molecule comprises phosphorothioate internucleoside linkages. In some embodiments, the siRNA molecule comprises phosphorothioate internucleoside linkages between at least two nucleosides. In some embodiments, the siRNA molecule comprises phosphorothioate internucleoside linkages between all nucleosides. For example, in some embodiments, the siRNA molecule comprises modified internucleoside linkages at the first, second, and/or (e.g., and) third internucleoside linkage at the 5′ or 3′ end of the siRNA molecule.

In some embodiments, the modified internucleoside linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of siRNA molecules described herein can be combined with each other. For example, one, two, three, four, five, or more different types of modifications can be included within the same siRNA molecule.

In some embodiments, the antisense strand comprises one or more modified nucleotides or nucleosides (e.g., 1, 2, 3, 4, 5, 6, 7, 8, 9, 10 or more). In some embodiments, the antisense strand comprises one or more modified nucleotides/nucleosides and/or (e.g., and) one or more modified internucleoside linkages. In some embodiments, the modified nucleotide/nucleoside comprises a modified sugar moiety (e.g., a 2′ modified nucleoside). In some embodiments, the antisense strand comprises one or more 2′ modified nucleosides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, each nucleotide/nucleoside of the antisense strand is a modified nucleotide/nucleoside (e.g., a 2′-modified nucleoside). In some embodiments, the antisense strand comprises one or more phosphorodiamidate morpholinos. In some embodiments, the antisense strand is a phosphorodiamidate morpholino oligomer (PMO).

In some embodiments, antisense strand contains a phosphorothioate or other modified internucleoside linkage. In some embodiments, the antisense strand comprises phosphorothioate internucleoside linkages. In some embodiments, the antisense strand comprises phosphorothioate internucleoside linkages between at least two nucleosides. In some embodiments, the antisense strand comprises phosphorothioate internucleoside linkages between all nucleosides. For example, in some embodiments, the antisense strand comprises modified internucleoside linkages at the first, second, and/or (e.g., and) third internucleoside linkage at the 5′ or 3′ end of the siRNA molecule. In some embodiments, the modified internucleoside linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the antisense strand described herein can be combined with each other. For example, one, two, three, four, five, or more different types of modifications can be included within the same antisense strand.

In some embodiments, the sense strand comprises one or more modified nucleotides or nucleosides (e.g., 1, 2, 3, 4, 5, 6, 7, 8, 9, 10 or more). In some embodiments, the sense strand comprises one or more modified nucleotides/nucleosides and/or (e.g., and) one or more modified internucleoside linkages. In some embodiments, the modified nucleotide/nucleoside comprises a modified sugar moiety (e.g., a 2′ modified nucleoside). In some embodiments, the sense strand comprises one or more 2′ modified nucleotides/nucleosides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, each nucleotide or nucleotide of the sense strand is a modified nucleotide/nucleoside (e.g., a 2′-modified nucleoside). In some embodiments, the sense strand comprises one or more phosphorodiamidate morpholinos. In some embodiments, the antisense strand is a phosphorodiamidate morpholino oligomer (PMO). In some embodiments, the sense strand contains a phosphorothioate or other modified internucleoside linkage. In some embodiments, the sense strand comprises phosphorothioate internucleoside linkages. In some embodiments, the sense strand comprises phosphorothioate internucleoside linkages between at least two nucleosides. In some embodiments, the sense strand comprises phosphorothioate internucleoside linkages between all nucleosides. For example, in some embodiments, the sense strand comprises modified internucleoside linkages at the first, second, and/or (e.g., and) third internucleoside linkage at the 5′ or 3′ end of the sense strand.

In some embodiments, the modified internucleoside linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the sense strand described herein can be combined with each other. For example, one, two, three, four, five, or more different types of modifications can be included within the same sense strand.

In some embodiments, the antisense or sense strand of the siRNA molecule comprises modifications that enhance or reduce RNA-induced silencing complex (RISC) loading. In some embodiments, the antisense strand of the siRNA molecule comprises modifications that enhance RISC loading. In some embodiments, the sense strand of the siRNA molecule comprises modifications that reduce RISC loading and reduce off-target effects. In some embodiments, the antisense strand of the siRNA molecule comprises a 2′-O-methoxyethyl (2′-MOE) modification. The addition of the 2′-O-methoxyethyl (2′-MOE) group at the cleavage site improves both the specificity and silencing activity of siRNAs by facilitating the oriented RNA-induced silencing complex (RISC) loading of the modified strand, as described in Song et al., (2017) Mol Ther Nucleic Acids 9:242-250, incorporated herein by reference in its entirety. In some embodiments, the antisense strand of the siRNA molecule comprises a 2′-OMe-phosphorodithioate modification, which increases RISC loading as described in Wu et al., (2014) Nat Commun 5:3459, incorporated herein by reference in its entirety.

In some embodiments, the sense strand of the siRNA molecule comprises a 5′-morpholino, which reduces RISC loading of the sense strand and improves antisense strand selection and RNAi activity, as described in Kumar et al., (2019) Chem Commun (Camb) 55(35):5139-5142, incorporated herein by reference in its entirety. In some embodiments, the sense strand of the siRNA molecule is modified with a synthetic RNA-like high affinity nucleoside analogue, Locked Nucleic Acid (LNA), which reduces RISC loading of the sense strand and further enhances antisense strand incorporation into RISC, as described in Elman et al., (2005) Nucleic Acids Res. 33(1): 439-447, incorporated herein by reference in its entirety. In some embodiments, the sense strand of the siRNA molecule comprises a 5′ unlocked nucleic acid (UNA) modification, which reduce RISC loading of the sense strand and improve silencing potency of the antisense strand, as described in Snead et al., (2013) Mol Ther Nucleic Acids 2(7):e103, incorporated herein by reference in its entirety. In some embodiments, the sense strand of the siRNA molecule comprises a 5-nitroindole modification, which decreased the RNAi potency of the sense strand and reduces off-target effects as described in Zhang et al., (2012) Chembiochem 13(13):1940-1945, incorporated herein by reference in its entirety. In some embodiments, the sense strand comprises a 2′-O′methyl (2′-O-Me) modification, which reduces RISC loading and the off-target effects of the sense strand, as described in Zheng et al., FASEB (2013) 27(10): 4017-4026, incorporated herein by reference in its entirety. In some embodiments, the sense strand of the siRNA molecule is fully substituted with morpholino, 2′-MOE or 2′-O-Me residues, and are not recognized by RISC as described in Kole et al., (2012) Nature reviews. Drug Discovery 11(2):125-140, incorporated herein by reference in its entirety. In some embodiments the antisense strand of the siRNA molecule comprises a 2′-MOE modification and the sense strand comprises an 2′-O-Me modification (see e.g., Song et al., (2017) Mol Ther Nucleic Acids 9:242-250). In some embodiments at least one (e.g., at least 2, at least 3, at least 4, at least 5, at least 10) siRNA molecule is linked (e.g., covalently) to a muscle-targeting agent. In some embodiments, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). In some embodiments, the muscle-targeting agent is an antibody. In some embodiments, the muscle-targeting agent is an anti-transferrin receptor 1 antibody (e.g., any one of the anti-TfR1 antibodies provided herein). In some embodiments, the muscle-targeting agent may be linked to the 5′ end of the sense strand of the siRNA molecule. In some embodiments, the muscle-targeting agent may be linked to the 3′ end of the sense strand of the siRNA molecule. In some embodiments, the muscle-targeting agent may be linked internally to the sense strand of the siRNA molecule. In some embodiments, the muscle-targeting agent may be linked to the 5′ end of the antisense strand of the siRNA molecule. In some embodiments, the muscle-targeting agent may be linked to the 3′ end of the antisense strand of the siRNA molecule. In some embodiments, the muscle-targeting agent may be linked internally to the antisense strand of the siRNA molecule.

w. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

x. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or (e.g., and) loop structures. The nucleic acid that forms the nucleic acid aptamer may comprise naturally occurring nucleotides/nucleosides, modified nucleotides/nucleosides, naturally occurring nucleotides/nucleosides with hydrocarbon linkers (e.g., an alkylene) or a polyether linker (e.g., a PEG linker) inserted between one or more nucleotides/nucleosides, modified nucleotides/nucleosides with hydrocarbon or PEG linkers inserted between one or more nucleotides/nucleosides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

y. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′, 5′-phosphate diester to a 2′, 3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

z. Guide Nucleic Acids

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid-programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csnl) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

aa. Multimers

In some embodiments, molecular payloads may comprise multimers (e.g., concatemers) of 2 or more oligonucleotides connected by a linker. In this way, in some embodiments, the oligonucleotide loading of a complex/conjugate can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

ii. Small Molecules

Any suitable small molecule may be used as a molecular payload, as described herein. In some embodiments, the small molecule promotes exon skipping of MSTN (e.g., exon 2 of MSTN) sequences. In some embodiments, the small molecule is as described in International Patent Application Publication WO2013137832A1, published Sep. 19, 2013, entitled “Myostatin inhibitors”; the contents of which is incorporated herein in its entirety. In some embodiments, the small molecule inhibits formation of an INHBA oligomer or dimer. In some embodiments, the small molecule inhibits formation of activin A and/or inhibin A. In some embodiments, the small molecule inhibits the function of Inhibin, beta A (INHBA).

In some embodiments, the small molecule is an ACVR1B inhibitor. In some embodiments, the small molecule is SB-431542 or a derivative of SB-431542. In some embodiments, the small molecule is AZ12601011 or a derivative of AZ12601011. In some embodiments, the small molecule is SB-505124 or a derivative of SB-505124. In some embodiments, the small molecule is as described in Sun, Z. et al., “The TGF-β Pathway Mediates Doxorubicin Effects on Cardiac Endothelial Cells.” J Mol Cell Cardiol. 2016 January; 90: 129-138.; Spender L. C., et al. “Preclinical Evaluation of AZ12601011 and AZ12799734, Inhibitors of Transforming Growth Factor β Superfamily Type 1 Receptors.” Mol Pharmacol. 2019 Feb; 95(2):222-234.; DaCosta Byfield S. et al., “SB-505124 is a selective inhibitor of transforming growth factor-beta type I receptors ALK4, ALK5, and ALK7.” Mol Pharmacol. 2004 Mar; 65(3):744-52.; Inman, G. J. et al., “SB-431542 is a potent and specific inhibitor of transforming growth factor-beta superfamily type I activin receptor-like kinase (ALK) receptors ALK4, ALK5, and ALK7.” Mol Pharmacol. 2002 July; 62(1):65-74.; the contents of each of which are incorporated herein in their entirety.

### MLCK1

In some embodiments, the small molecule inhibits the expression of MLCK1. In some embodiments, the small molecule inhibits the function of MLCK1. In some embodiments, the small molecule binds to the catalytic kinase domain of MLCK1. In some embodiments, the small molecule inhibits the catalytic kinase activity of MLCK1. In some embodiments, the small molecule binds to a non-catalytic domain of MLCK1. In some embodiments, the small molecule inhibits MLCK1 recruitment to the perijunctional actomyosin ring (PAMR). In some embodiments, the small molecule inhibits MLCK1 recruitment to the perijunctional actomyosin ring (PAMR) and does not inhibit the catalytic kinase activity of MLCK1.

In some embodiments, the small molecule is divertin, or a derivative thereof. In some embodiments, the small molecule is rucaparib, ML-9, an isoquinoline, an isoquinoline alkaloid, berberine, or a derivative thereof

In some embodiments, the small molecule is as described in Vallen Graham, W. et. al., “Intracellular MLCK1 diversion reverses barrier loss to restore mucosal homeostasis.” Nature Medicine volume 25, 690-700, 2019.; McCrudden, C. M. et al., “Vasoactivity of rucaparib, a PARP-1 inhibitor, is a complex process that involves myosin light chain kinase, P2 receptors, and PARP itself.” PLoS One. 2015 Feb. 17; 10(2):e0118187.; Nakanishi, S. et al., “MS-347a, a new inhibitor of myosin light chain kinase from Aspergillus sp. KY52178.” J Antibiot (Tokyo). 1993 December; 46(12):1775-81.; Xu, Z. et al. “Berberine Depresses Contraction of Smooth Muscle via Inhibiting Myosin Light-chain Kinase.” Pharmacogn Mag. 2017 Jul-Sep; 13(51):454-458.; Saitoh M. et al., “Selective inhibition of catalytic activity of smooth muscle myosin light chain kinase.” J Biol Chem. 1987 Jun. 5; 262(16):7796-801.; Nakanishi S. et al., “K-252a, a novel microbial product, inhibits smooth muscle myosin light chain kinase.” J Biol Chem. 1988 May 5; 263(13):6215-9.; Kerendi F. et al., “Inhibition of myosin light chain kinase provides prolonged attenuation of radial artery vasospasm.” Eur J Cardiothorac Surg. 2004 Dec; 26(6):1149-55.; and Nakanishi S. et al., “Wortmannin, a microbial product inhibitor of myosin light chain kinase.” J Biol Chem. 1992 Feb. 5; 267(4):2157-63.; the contents of each of which are incorporated herein in their entirety.

### ACVR1

In some embodiments, the small molecule is as described in Mohedas, A. H. et al. “Development of an ALK2-biased BMP type I receptor kinase inhibitor”, ACS Chem Biol. 2013; 8(6):1291-302.; Sanvitale, C. E. et al., “A new class of small molecule inhibitor ofBMP signaling”, PLoS One. 2013 Apr. 30; 8(4):e62721.; Kausar T. and S. M. Nayeem, “Identification of small molecule inhibitors of ALK2: a virtual screening, density functional theory, and molecular dynamics simulations study”, J Mol Model. 2018 Aug. 29; 24(9):262.; and International Patent Application Publication WO 2015/152183, published Nov. 8, 2015, “Prophylactic agent and therapeutic agent for fibrodysplasia ossificans progressive”; the entire contents of each of which are incorporated herein by reference. In some embodiments, the small molecule is as described in US Patent Application 2016/0075772, published Mar. 17, 2016, “Treatment of Fibrodysplasia Ossificans Progressiva”. In some embodiments, the small molecule is as described in WO 2016/121680, published Aug. 4, 2016, “Therapeutic agentfor fibrodysplasia ossificans progressiva”. In some embodiments, the small molecule is rapamycin, temsirolimus, everolimus, ridaforolimus, TAFA93, umirolimus, olcorolimus, zotarolimus, or a related compound. In some embodiments, the small molecule is as described in US Patent Application 2018/031579, published Feb. 1, 2018, “Momelotinibfor treating of acvr1-mediated diseases”. In some embodiments, the small molecule inhibits the expression of activin. In some embodiments, the small molecule inhibits the interaction between activin and activin A receptor type I. In some embodiments, the small molecule inhibits the expression or enzymatic activity of ACVR1. In some embodiments, the small molecule inhibits the signaling downstream of ACVR1. In some embodiments, the small molecule is an activin receptor type 2B (ACVR2B) antagonist used in combination with an activin receptor type 2A protein or peptide. In some embodiments, the small molecule is as described in Katagiri, T. et al. “Heterotopic bone induction via BMP signaling: Potential therapeutic targets for fibrodysplasia ossificans progressiva” Bone, 2017. In some embodiments, the small molecule is a RARγ agonist, e.g., Palovarotene. Additional examples of small molecules are included in Cappato, S. et al. “The Horizon of a Therapy for Rare Genetic Diseases: A “Druggable” Future for Fibrodysplasia Ossificans Progressiva” Int. J. Mol. Sci. 2018, 19(4), 989; Cappato, S et al, “High-throughput screening for modulators of ACVR1 transcription: discovery of potential therapeutics for fibrodysplasia ossificans progressiva.” Dis Model Mech. 2016 Jun. 1; 9(6):685-96.; and Kaplan, F. S. et al “Early clinical observations on the use of imatinib mesylate in FOP: A report of seven cases.” Bone. 2018 April; 109:276-280. The contents of each of the foregoing are incorporated herein by reference in their entireties. In some embodiments, the small molecule inhibitor of ACVR1 used in the complexes described herein is commercially available, e.g., DMH1 from Sigma-Aldrich (catalog #D8946).

### FBXO32

In some embodiments, the small molecule inhibits FBXO32 expression. In some embodiments, the small molecule inhibits the interaction between FBXO32 and one or more substrates. In some embodiments, the small molecule inhibits the interaction between FBXO32 and one or more proteins to be ubiquitinated and/or between FBXO32 and one or more components of the SCF complex. In some embodiments, the small molecule is gnetin C. In some embodiments, the small molecule is as described in JP201514031, which was granted on Nov. 1, 2017.

### TRIM63

In some embodiments, the small molecule disrupts the interaction between TRIM63 and one or more protein substrates. In some embodiments, the protein substrate is titin. In some embodiments, the small molecule inhibits TRIM63-dependent substrate ubiquitination. Non-limiting examples of small molecules targeting TRIM63 and/or TRIM63 activity are described in Bowen et al., J Cachexia Sarcopenia Muscle. 2017 December; 8(6):939-953; Adams et al., J Cachexia Sarcopenia Muscle. 2019 October; 10(5):1102-1115; and Eddins et al., Cell Biochem Biophys. 2011 June; 60(1-2):113-8. The contents of each of these publications listed above are incorporated herein in their entirety.

In some embodiments, the small molecule is lithium (e.g., as described in Linseman et al., J Neurochem. 2003 June; 85(6):1488-99, incorporated herein by reference). In some embodiments, the small molecule is Bis(3)-cognitin (e.g., as described in Yao et al., The Journal of Biological Chemistry, 287, 34246-34255, incorporated herein by reference). In some embodiments, the small molecule is retinoic acid (e.g., as described in Ma et al., Cancer Res; 74(5) Mar. 1, 2014, incorporated herein by reference).

### KLF15

In some embodiments, the small molecule increases the expression or function of KLF15. In some embodiments, the small molecule inhibits the expression or function of a protein or gene that represses KLF15 expression or function. In other embodiments, the small molecule decreases the expression or function of KLF15.

### MED1, MED13

In some embodiments, a small molecule for targeting MED1 or MED13 is a small molecule that specifically modulates protein-protein interactions between MED1 or MED13 and its interaction partners (e.g., other subunits of the Mediator complex, bromodomain-containing proteins such as BRD4, or CBP/p300). A small molecule that specifically modulates protein-protein interactions between MED1 or MED13 and its interaction partners may be a small molecule that binds to MED1 or MED13 and/or a small molecule that binds to the interaction partner. In some embodiments, a small molecule for targeting MED1 or MED13 is a small molecule that targets an epigentic modulator of MED1 or MED13 and/or epigenetic readers (e.g., BRD4). In some embodiments, a small molecule for targeting MED1 is small molecule that binds to BRD4 (e.g., JQ1 or a derivative of JQ1). In some embodiments, the small molecule is as described in Hermann, H. et al. “Small-molecule inhibition of BRD4 as a new potent approach to eliminate leukemic stem- and progenitor cells in acute myeloid leukemia AML” Oncotarget. 2012 December; 3(12):1588-99.; and International Patent Application WO2017/027571, entitled “Mechanism of resistance to bet bromodomain inhibitors”, which was published on Feb. 16, 2017; the contents of each of these publications listed above are incorporated herein in their entirety.

iii. Peptides/Proteins

Any suitable peptide or protein may be used as a molecular payload, as described herein. In some embodiments, a protein is an enzyme. These peptides or proteins may be produced, synthesized, and/or derivatized using several methodologies, e.g., phage displayed peptide libraries, one-bead one-compound peptide libraries, or positional scanning synthetic peptide combinatorial libraries. In some embodiments, the peptide or protein may comprise about 2-25 amino acids, about 2-20 amino acids, about 2-15 amino acids, about 2-10 amino acids, or about 2-5 amino acids. The peptide or protein may comprise naturally occurring amino acids, e.g., cysteine, alanine, or non-naturally occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, the peptide may be linear; in other embodiments, the peptide may be cyclic, e.g., bicyclic.

### MSTN

In some embodiments, the protein or peptide is as described in International Patent Application Publication WO2014119753A1, published on Aug. 7, 2014, entitled “Myostatin-inhibiting peptide”; International Patent Application Publication WO2004058988A2, published on Jul. 15, 2004, entitled “Binding agents which inhibit myostatin”; International Patent Application Publication WO2012024242A1, published on Feb. 23, 2012, entitled “Antibodies that bind myostatin, compositions and methods”; Takayama, K. et. al. “Chain-Shortened Myostatin Inhibitory Peptides Improve Grip Strength in Mice” ACS Med Chem Lett. 2019 May 28; 10(6):985-990.; Jin, Q. et. al. “A GDF11/myostatin inhibitor, GDF11 propeptide-Fc, increases skeletal muscle mass and improves muscle strength in dystrophic mdx mice” Skelet Muscle. 2019 May 27; 9(1):16.; Long, K. K. et. al., “Specific inhibition of myostatin activation is beneficial in mouse models of SMA therapy” Hum Mol Genet. 2019 Apr. 1; 28(7):1076-1089.; Campbell, C. et. al. “Myostatin inhibitor ACE-031 treatment of ambulatory boys with Duchenne muscular dystrophy: Results of a randomized, placebo-controlled clinical trial” Muscle Nerve. 2017 April; 55(4):458-464.; and Takayama, K. et. al., “Effect of N-Terminal Acylation on the Activity of Myostatin Inhibitory Peptides” ChemMedChem. 2016 Apr. 19; 11(8):845-9.; the contents of each of these publications listed above are incorporated herein in their entirety.

In some embodiments, a peptide or protein that targets MSTN selectively inhibits the activity of myostatin proteins. In some embodiments, a peptide or protein that targets MSTN selectively inhibits the activity of myostatin proteins comprises 10-50, 20-50, 20-40, 20-30, 10-100, 25-100, 50-100, or more than 100 amino acids. In some embodiments, a peptide or protein that targets MSTN is a Growth differentiation factor 11 (GDF11) polypeptide (e.g., a GDF11 propeptide-Fc fusion). In some embodiments, a peptide or protein that targets MSTN is a fusion protein of activin receptor type IIB and IgG1-Fc. In some embodiments, a peptide or protein that targets MSTN is a follistatin polypeptide (e.g., a recombinant mutant follistatin) that inhibits activity of myostatin protein. A follistatin polypeptide may comprise a follistatin N-terminal domain, a follistatin-1 domain, a follistatin-2 domain, a follistatin-3 domain and/or a follistatin C-terminal domain. In some embodiments, a peptide or protein that targets MSTN is an anti-MSTN antibody.

### INHBA

In some embodiments, a peptide or protein is as described in Chen, J. L. et al. “Development of Novel Activin-Targeted Therapeutics” Mol Ther. 2015 March; 23(3): 434-444.; Hu, J. et al. “Activin A inhibition attenuates sympathetic neural remodeling following myocardial infarction in rats” Mol Med Rep. 2018 April; 17(4): 5074-5080.; Yaden, B C et al. “Inhibition of activin A ameliorates skeletal muscle injury and rescues contractile properties by inducing efficient remodeling in female mice” Am J Pathol. 2014 April; 184(4):1152-66.; U.S. Patent Application Publication US20180273599, published on Sep. 27, 2018, and entitled “Inhibin Analogs”; the entire contents of which is incorporated herein in its entirety.

In some embodiments, a peptide or protein that targets INHBA selectively inhibits the formation of oligomers or dimers comprising INHBA. In some embodiments, the peptide or protein inhibits formation of activin A and/or inhibin A. In some embodiments, a peptide or protein that targets INHBA selectively inhibits the function of Inhibin, beta A (INHBA). In some embodiments, a peptide or protein that targets INHBA is a modified activin A and/or activin B prodomain. In some embodiments, a peptide or protein that targets INHBA is follistatin or a derivative thereof. A peptide or protein that targets INHBA comprises 10-50, 20-50, 20-40, 20-30, 10-100, 25-100, 50-100, or more than 100 amino acids. In some embodiments, a peptide or protein that targets INHBA is an Inhibin analog. In some embodiments, a peptide or protein that targets INHBA is an anti-INHBA antibody.

### ACVR1

In some embodiments, a protein is a truncated ACVR1B protein. In some embodiments, a truncated ACVR1B protein competes with endogenous, full-length ACVR1B for binding to activin receptor type-2 proteins. In some embodiments, a truncated ACVR1B protein cannot be phosphorylated. A truncated ACVR1B protein that cannot be phosphorylated cannot transduce activin signaling. In some embodiments, a truncated ACVR1B protein is truncated at its C-terminal end. A truncated ACVR1B protein may lack most of subdomain XI of full-length ACVR1, may lack subdomains X and XI of full-length ACVR1, or may lack kinase subdomains IX-XI and part of subdomain VIII of full-length ACVR1.

In some embodiments, the protein or peptide is as described in Zhou, Y. et al. “Truncated Activin Type I Receptor Alk4 Isoforms Are Dominant Negative Receptors Inhibiting Activin Signaling.” Molecular Endocrinology, 2000, 14:12, 2066-2075.; International Patent Application Publication WO 2016/161477, entitled “A method of treating neoplasias”, filed on Mar. 23, 2016; the contents of each of these publications listed above are incorporated herein in their entirety.

### MLCK1

In some embodiments, the peptide or protein inhibits the expression of MLCK1. In some embodiments, the peptide or protein inhibits the function of MLCK1. In some embodiments, the peptide or protein binds to the catalytic kinase domain of MLCK1. In some embodiments, the peptide or protein inhibits the catalytic kinase activity of MLCK1. In some embodiments, the peptide or protein binds to a non-catalytic domain of MLCK1. In some embodiments, the peptide or protein inhibits MLCK1 recruitment to the perijunctional actomyosin ring (PAMR). In some embodiments, the peptide or protein inhibits MLCK1 recruitment to the perijunctional actomyosin ring (PAMR) and does not inhibit the catalytic kinase activity of MLCK1.

In some embodiments, the protein or peptide is as described in Ikebe M. et al., “Primary structure required for the inhibition of smooth muscle myosin light chain kinase.” FEBS Lett. 1992 Nov. 9; 312(2-3):245-8.; and Hunt, J. T. et al., “Minimum requirements for inhibition of smooth-muscle myosin light-chain kinase by synthetic peptides.” Biochem J. 1989 Jan. 1; 257(1):73-8.; the contents of each of which are incorporated herein in their entirety.

### ACVR1

In some embodiments, the peptide or protein for modulating ACVR1 activity is a BMP inhibitor such as regulatory SMAD 6 and 7 or fragment thereof. Additional examples of peptides or proteins are included in Cappato, S. et al. “The Horizon of a Therapy for Rare Genetic Diseases: A “Druggable” Future for Fibrodysplasia Ossificans Progressiva” Int. J. Mol. Sci. 2018, 19(4), 989. The contents of each of the foregoing are incorporated herein by reference in their entireties.

### FBXO32

As a non-limiting example, the protein or peptide is an anti-FBXO32 antibody, PGC-1α, or PGC-I β. In some embodiments, the protein or peptide is as described in Bdolah et al., Am J Physiol Regul Integr Comp Physiol. 2007 February; 292(2):R971-6; Sandri et al., Cell 117:399-412, 2006; and WO2008156561, entitled, “METHODS AND COMPOSITIONS FOR THE TREATMENT AND DIAGNOSIS OF STATIN-INDUCED MYOPATHY,” which was published on Dec. 24, 2008. The contents of each of these publications listed above are incorporated herein in their entirety.

### TRIM63

In some embodiments, the protein or peptide is a TRIM63 antibody. See, e.g., de Palma et al., Joint Bone Spine. 2008 January; 75(1):53-7; and Clarke et al., Cell Metab. 2007 Nov; 6(5):376-85. The contents of each of these publications listed above are incorporated herein in their entirety.

### MEF2D

In some embodiments, a protein or peptide is a MEF2D antibody (e.g., as described in Li et al., Journal of Neuroscience 1 Sep. 2001, 21 (17) 6544-6552, incorporated herein by reference). MEF2D antibodies are also commercially available, e.g., from Abcam (catalog #AB32845) and Thermo Fisher Scientific (catalog #MA5-268909).

### KLF15

In some embodiments, the peptide or protein increases the expression or function of KLF15. In some embodiments, the peptide or protein inhibits the expression or function of a protein or gene that represses KLF15 expression or function. In other embodiments, the peptide or protein decreases the expression or function of KLF15.

In some embodiments, a peptide or protein is a KLF15 peptide or protein. In some embodiments, the KLF15 peptide or protein functions in the same manner as endogenous KLF15 (e.g., to inhibit cardiac transcription factors, such as MEF2, GATA4, and myocardin). In some embodiments, the KLF15 peptide or protein has at least 50% of the functional activity of endogenous KLF15 (e.g., at least 60%, 70%, 80%, 90%, or more). In some embodiments, the KLF15 peptide or protein is a full-length KLF15 protein.

### MED1, MED13

In some embodiments, a peptide or protein for targeting MED1 or MED13 is a peptide or protein that specifically modulates protein-protein interactions between MED1 or MED13 and its interaction partners (e.g., other subunits of the Mediator complex, bromodomain-containing proteins such as BRD4, or CBP/p300). A peptide or protein that specifically modulates protein-protein interactions between MED1 or MED13 and its interaction partners may be a peptide or protein that binds to MED1 or MED13 and/or a small molecule that binds to the interaction partner. In some embodiments, a peptide or protein for targeting MED1 or MED13 is an antibody (e.g., an anti-MED1 antibody or an anti-MED13 antibody).

iv. Nucleic Acid Constructs

Any suitable gene expression construct may be used as a molecular payload, as described herein. In some embodiments, a gene expression construct may be a vector or a cDNA fragment. In some embodiments, a gene expression construct may be messenger RNA (mRNA). In some embodiments, a mRNA used herein may be a modified mRNA, e.g., as described in U.S. Pat. No. 8,710,200, issued on Apr. 24, 2014, entitled “Engineered nucleic acids encoding a modified erythropoietin and their expression”. In some embodiments, a mRNA may comprise a 5′ methyl cap. In some embodiments, a mRNA may comprise a polyA tail, optionally of up to 160 nucleotides in length.

A gene expression construct may encode a sequence of a protein that reduces the expression or activity of myostatin. A gene expression construct may encode a sequence of a protein that is a peptide or protein analog of INHBA that inhibits or disrupts the formation of INHBA dimers or oligomers. In some embodiments, the gene expression construct inhibits or disrupts the formation of activin A and/or inhibin A. A gene expression construct may encode a sequence of a protein that is a truncated ACVR1B protein. In some embodiments, a truncated ACVR1B protein competes with endogenous, full-length ACVR1B for binding to activin receptor type-2 proteins. In some embodiments, a truncated ACVR1B protein cannot be phosphorylated. A truncated ACVR1B protein that cannot be phosphorylated cannot transduce activin signaling. In some embodiments, a truncated ACVR1B protein is truncated at its C-terminal end. A truncated ACVR1B protein may lack most of subdomain XI of full-length ACVR1, may lack subdomains X and XI of full-length ACVR1, or may lack kinase subdomains IX-XI and part of subdomain VIII of full-length ACVR1. In some embodiments, the gene expression construct may be expressed, e.g., overexpressed, within the nucleus of a muscle cell. In some embodiments, the gene expression construct encodes a Growth differentiation factor 11 (GDF11) polypeptide (e.g., a GDF11 propeptide-Fc fusion), a fusion protein of activin receptor type IIB and IgG1-Fc, a follistatin polypeptide (e.g., a recombinant mutant follistatin, e.g., comprising a follistatin N-terminal domain, a follistatin-1 domain, a follistatin-2 domain, a follistatin-3 domain and/or a follistatin C-terminal domain), or an anti-MSTN antibody. In some embodiments, the gene expression constructs encode a protein that comprises at least one zinc finger. In some embodiments, the gene expression construct encodes a protein that leads to a reduction in the expression of a MSTN gene. In some embodiments, the gene expression construct encodes a protein that leads to a reduction in the expression of an INHBA gene. In some embodiments, the gene expression construct encodes a protein that binds to an ACVR1B gene. In some embodiments, the gene expression construct encodes a protein that leads to a reduction in the expression of an ACVR1B gene.

### MLCK1

A gene expression construct may encode a sequence of a protein that inhibits the expression of MLCK1. In some embodiments, the gene expression construct may encode a protein that inhibits the function of MLCK1. In some embodiments, the gene expression construct may encode a protein that binds to the catalytic kinase domain of MLCK1. In some embodiments, the gene expression construct may encode a protein that inhibits the catalytic kinase activity of MLCK1. In some embodiments, the gene expression construct may encode a protein that binds to a non-catalytic domain of MLCK1. In some embodiments, the gene expression construct may encode a protein that inhibits MLCK1 recruitment to the perijunctional actomyosin ring (PAMR). In some embodiments, the gene expression construct may encode a protein inhibits MLCK1 recruitment to the perijunctional actomyosin ring (PAMR) and does not inhibit the catalytic kinase activity of MLCK1. In some embodiments, the gene expression construct may be expressed, e.g., overexpressed, within the nucleus of a muscle cell. In some embodiments, the gene expression constructs encode a protein that comprises at least one zinc finger. In some embodiments, the gene expression construct encodes a protein that leads to a reduction in the expression of a MLCK1.

### ACVR1

A gene expression construct may encode a sequence of a protein that leads to decreased expression of ACVR1 gene or decreased activity of ACVR1 protein. In some embodiments, the gene expression construct may be expressed, e.g., overexpressed, within the nucleus of a muscle cell. In some embodiments, the gene expression construct encodes a protein that inhibit the function of epigenetic regulators that regulate the expression of ACVR1. In some embodiments, the gene expression construct encodes a protein that leads to a reduction in the expression of an epigenetic regulators that negatively regulate the expression of ACVR1, e.g., histone deactylases. In some embodiments, the gene expression construct encodes a gene editing enzyme. In some embodiments, the gene expression construct encodes activin. In some embodiments, the gene expression construct encodes a protein capable of inhibiting the function of ACVR1 protein. In some embodiments, the gene expression construct encodes an oligonucleotide (e.g., shRNA) that inhibits expression of ACVR1. In some embodiments, the gene expression construct encodes a protein that leads to a reduction in the expression of a ACVR1 gene.

### FBXO32

In some embodiments, a gene expression construct may encode FBXO32. In some embodiments, a gene expression construct may encode a sequence of a protein that inhibits the expression of FBXO32 or inhibits FBOX32 activity. As a non-limiting example, PGC-1α expression has been shown to inhibit FBXO32 expression. See, e.g., Sandri et al., Cell 117:399-412, 2006 and WO2008156561, entitled, “METHODS AND COMPOSITIONS FOR THE TREATMENT AND DIAGNOSIS OF STATIN-INDUCED MYOPATHY,” which was published on Dec. 24, 2008. In some instances, a molecular payload is a nucleic acid encoding PGC-I β. See, e.g., WO2008156561, entitled, “METHODS AND COMPOSITIONS FOR THE TREATMENT AND DIAGNOSIS OF STATIN-INDUCED MYOPATHY,” which was published on Dec. 24, 2008. In some instances, a molecular payload is a nucleic acid encoding a FBXO32 antibody. In some embodiments, the gene expression construct may be expressed, e.g., overexpressed, within the nucleus of a muscle cell. In some embodiments, the gene expression constructs encode a protein that comprises at least one zinc finger. In some embodiments, the gene expression construct encodes a protein that leads to a reduction in the expression of a FBXO32.

### TRIM63

A gene expression construct may encode a sequence of a protein that is a TRIM63 antibody. In some embodiments, the gene expression construct may be expressed, e.g., overexpressed, within the nucleus of a muscle cell. In some embodiments, the gene expression construct encodes a TRIM63 antibody. In some embodiments, the gene expression constructs encode a protein that comprises at least one zinc finger. In some embodiments, the gene expression construct encodes a protein that binds to a TRIM63 and/or prevents the interaction between TRIM63 and one or more TRIM63 substrates (e.g., titin). In some embodiments, the gene expression construct encodes a protein that leads to a reduction in the expression of a TRIM63.

### MEF2D, KLF15, MED1, MED13, or PPP1R3A

A gene expression construct may encode MEF2D, KLF15, MED1, MED13, or PPP1R3A. A gene expression construct may encode a peptide or protein targeting MEF2D, KLF15, MED1, MED13, or PPP1R3A.

In some embodiments, the gene expression construct encodes a specific isoform of MEF2D. In some embodiments, the gene expression construct encodes a protein that binds to MEF2D or its binding partner (e.g., Hsc70, as described in Lee et al., J. Biol. Chem. 285: 33779-33787, 2010, incorporated herein by reference) to disrupt the interaction between MEF2D and its binding partner. In some embodiments, the gene expression construct encodes a protein that leads to a reduction in the expression of a MEF2D. In some embodiments, the gene expression construct encodes an MEF2D antibody.

In some embodiments, the gene expression construct encodes a specific isoform of KLF15. In some embodiments, the KLF15 peptide or protein functions in the same manner as endogenous KLF15 (e.g., to inhibit cardiac transcription factors, such as MEF2, GATA4, and myocardin). In some embodiments, the KLF15 peptide or protein has at least 50% of the functional activity of endogenous KLF15 (e.g., at least 60%, 70%, 80%, 90%, or more). In some embodiments, the KLF15 peptide or protein is a full-length KLF15 protein. In some embodiments, the gene expression construct encodes a protein that binds to a cardiac transcription factor, such as MEF2, GATA4, or myocardin, for inhibition of these cardiac transcription factors and their function. In some embodiments, the gene expression construct encodes a protein that leads to an increase in the expression of a KLF15.

In some embodiments, the gene expression construct encodes a peptide or protein for targeting MED1 (e.g., an anti-MED1 antibody). In some embodiments, the gene expression construct encodes a protein that binds to MED1 or an interaction partner of MED1 (e.g., other subunits of the Mediator complex, bromodomain-containing proteins such as BRD4, or CBP/p300). In some embodiments, the gene expression construct encodes MED1 or a portion of MED1. In some embodiments, the gene expression construct encodes a protein that changes the expression of a MED1 gene (e.g., increases expression of a MED1 gene).

A gene expression construct may encode a sequence of a protein that encodes a peptide or protein for targeting MED13 (e.g., an anti-MED13 antibody). In some embodiments, the gene expression construct may be expressed, e.g., overexpressed, within the nucleus of a muscle cell. In some embodiments, the gene expression constructs encode a protein that comprises at least one zinc finger. In some embodiments, the gene expression construct encodes a protein that binds to MED13 or an interaction partner of MED13 (e.g., cyclin C, CDK8, MED12). In some embodiments, the gene expression construct encodes a protein that leads to a reduction in the expression of a mutant MED13 gene. In some embodiments, the gene expression construct encodes a protein that leads to an increase in the expression of a wild-type MED13 gene. In some embodiments, the gene expression construct encodes MED13, or a portion of MED13.

In some embodiments, the gene expression construct may be expressed, e.g., overexpressed, within the nucleus of a muscle cell. In some embodiments, the gene expression construct encodes a protein that comprises at least one zinc finger. In some embodiments, the gene expression construct encodes a gene editing enzyme. Additional examples of nucleic acid constructs that may be used as molecular payloads are provided in International Patent Application Publication WO2017152149A1, published on Sep. 19, 2017, entitled, “CLOSED-ENDED LINEAR DUPLEX DNA FOR NON-VIRAL GENE TRANSFER”; U.S. Pat. No. 8,853,377B2, issued on Oct. 7, 2014, entitled, “MRNA FOR USE IN TREATMENT OF HUMAN GENETIC DISEASES”; and US Patent U.S. Pat. No. 8,822,663B2, issued on Sep. 2, 2014, ENGINEERED NUCLEIC ACIDS AND METHODS OF USE THEREOF,” the contents of each of which are incorporated herein by reference in their entireties.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g., Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally occurring amino acids, e.g., cysteine, alanine, or non-naturally occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g., cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

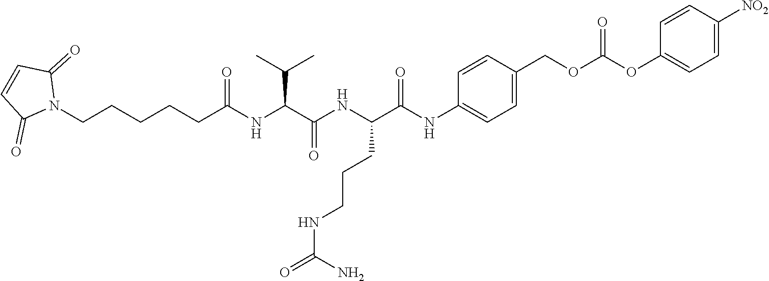 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

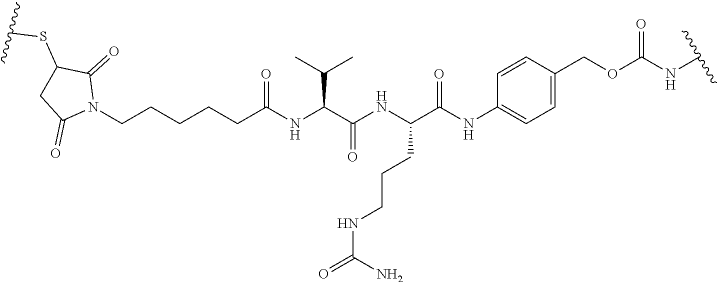 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1]

In some embodiments, before conjugation, a linker comprises a structure of:

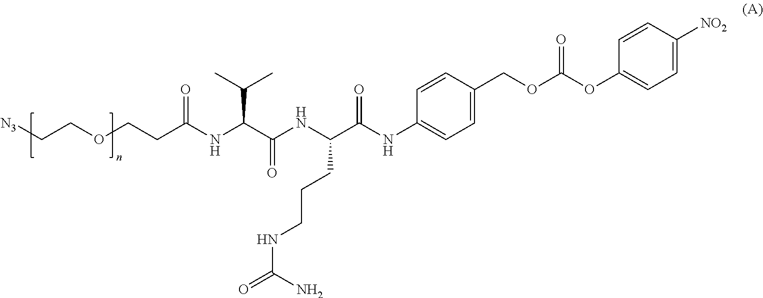

wherein n is any number from 0-10. In some embodiments, n is 3.

In some embodiments, a linker comprises a structure of:

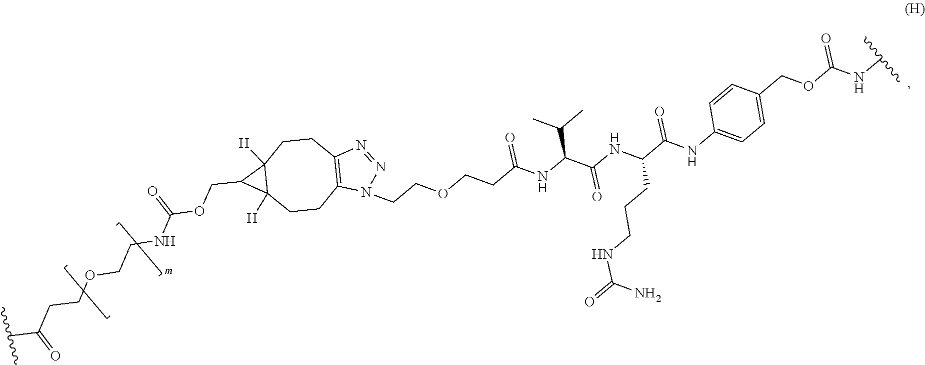

wherein n is any number from 0-10, wherein m is any number from 0-10. In some embodiments, n is 3 and/or (e.g., and) m is 4.

In some embodiments, a linker comprises a structure of:

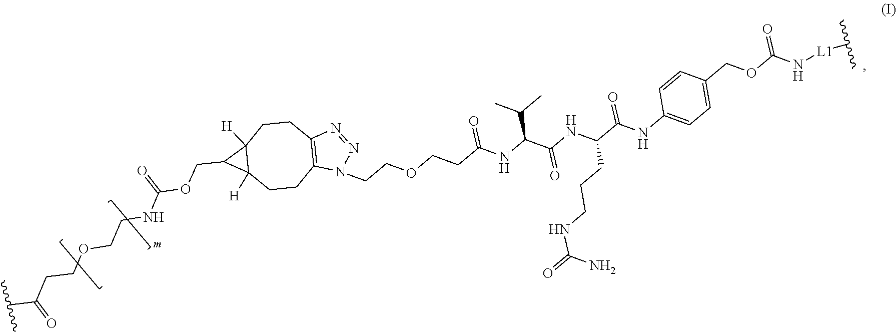

wherein n is any number from 0-10, wherein m is any number from 0-10. In some embodiments, n is 3 and/or (e.g., and) m is 4.
ii. Non-cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)ₙ sequence (see, e.g., Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S,; an optionally substituted heterocyclylene further comprising at least one heteroatom selected from N, O, and S, an imino, an optionally substituted nitrogen species, an optionally substituted oxygen species O, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker Conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g., a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)—N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)—N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reaction between a nucleophile, e.g., an amine or a hydroxyl group, and an electrophile, e.g., a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

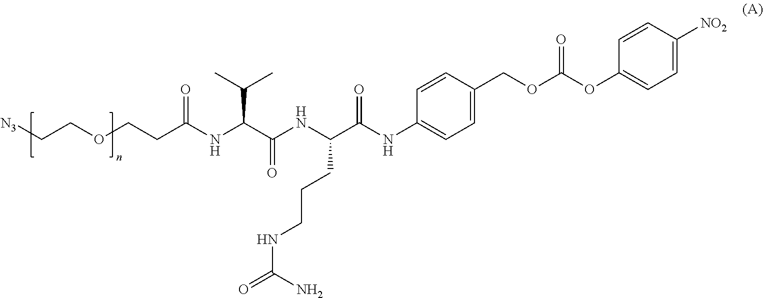

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of:

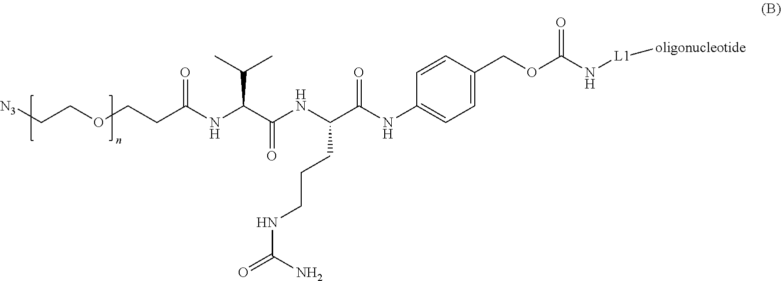

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

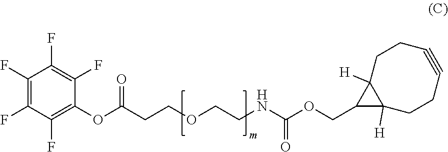

wherein m is any number from 0-10. In some embodiments, m is 4.

In some embodiments, the azide of the compound of structure (B) forms a triazole via a click reaction with the alkyne of the compound of structure (C), forming a compound comprising a structure of:

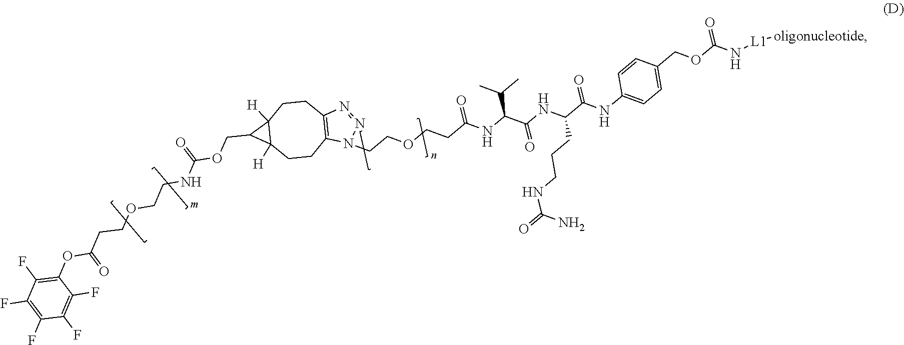

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

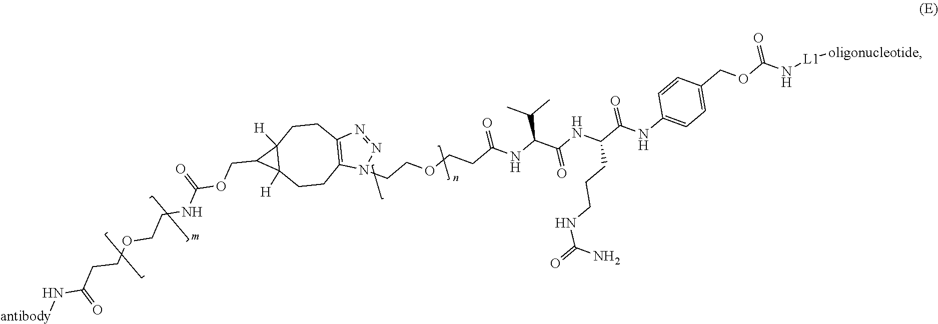

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

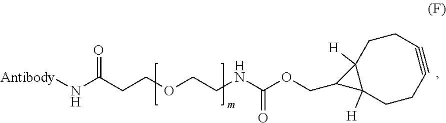

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

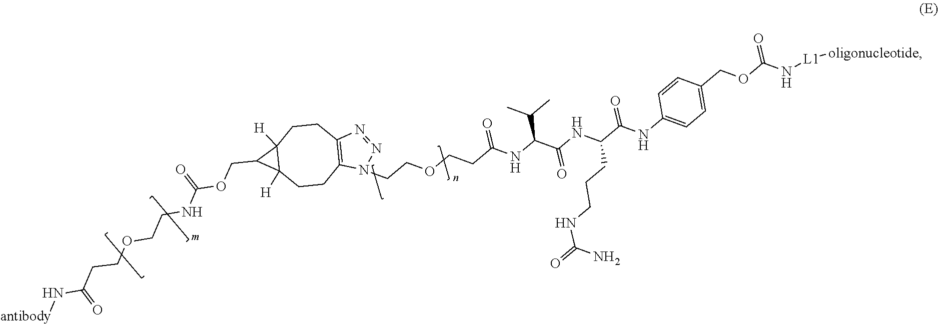

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising: a structure of:

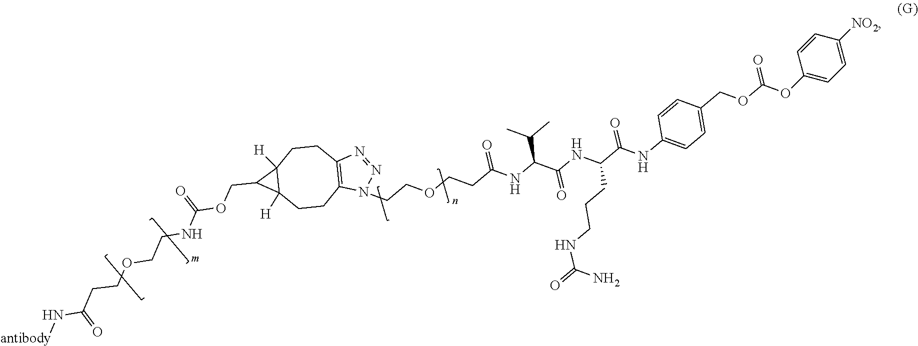

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

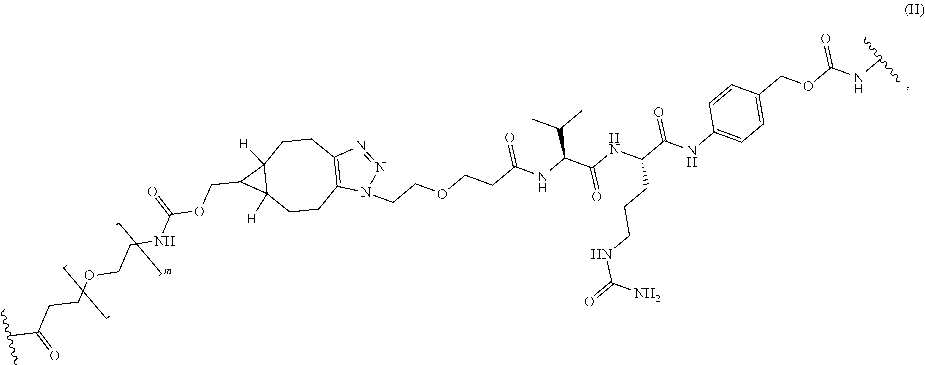

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

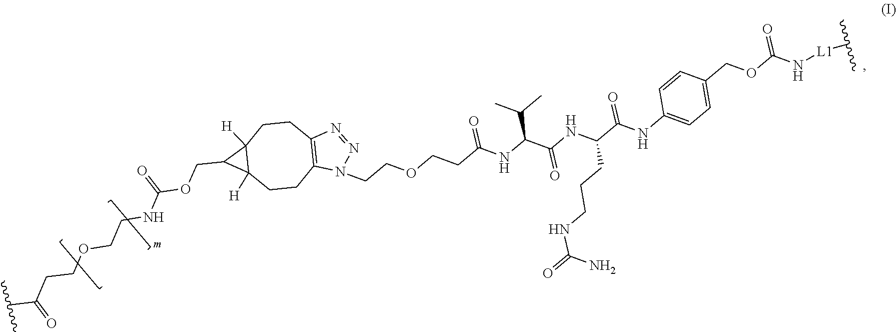

wherein n is any number from 0-10, wherein m is any number from 0-10. In some embodiments, n is 3 and/or (e.g., and) m is 4.

In some embodiments, in formulae (B), (D), (E), and (I), L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}-. —NR^{A}S(O)₂—, or a combination thereof, wherein each R^{A} is independently hydrogen or substituted or unsubstituted alkyl. In some embodiments, L1 is

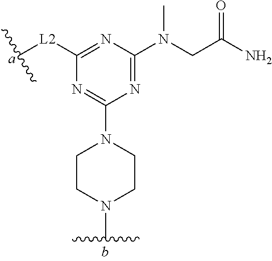

wherein L2 is

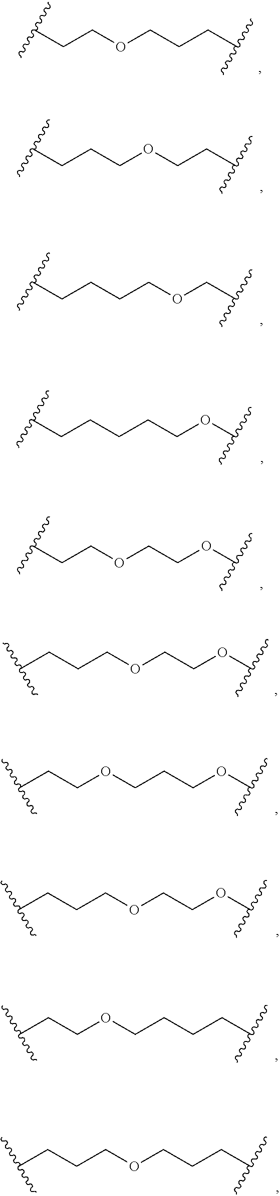

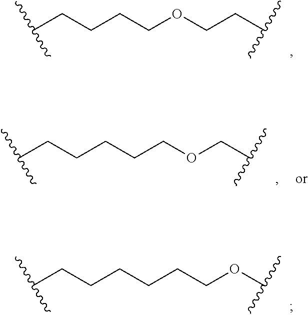 [SMILES: *CCCCOCC* ; *CCCCCOC* ; *CCCCCCO*]

wherein a labels the site directly linked to the carbamate moiety of formulae (B), (D), (E), and (I); and b labels the site covalently linked (directly or via additional chemical moieties) to the oligonucleotide.

In some embodiments, L1 is:

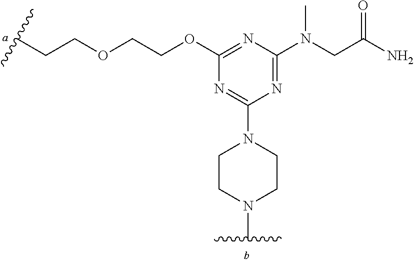 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

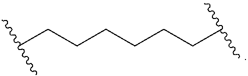 [SMILES: *CCCCCC*]

In some embodiments, L1 is linked to a 5′ phosphate of the oligonucleotide. In some embodiments, L1 is linked to a 5′ phosphorothioate of the oligonucleotide. In some embodiments, L1 is linked to a 5′ phosphonoamidate of the oligonucleotide.

In some embodiments, L1 is linked to a 5′ phosphate of the oligonucleotide. In some embodiments, the linkage of L1 to a 5′ phosphate of the oligonucleotide forms a phosphodiester bond between L1 and the oligonucleotide.

In some embodiments, L1 is linked to a 3′ phosphate of the oligonucleotide. In some embodiments, the linkage of L1 to a 3′ phosphate of the oligonucleotide forms a phosphodiester bond between L1 and the oligonucleotide.

In some embodiments, L1 is optional (e.g., need not be present).

In some embodiments, any one of the complexes described herein has a structure of:

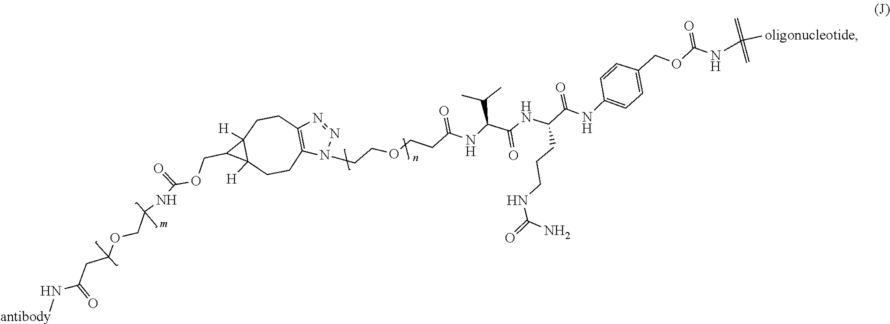

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

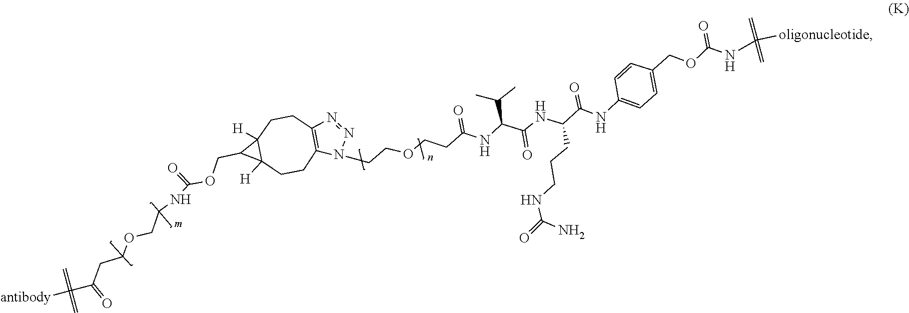

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Other aspects of the present disclosure provide complexes comprising any one the muscle targeting agent (e.g., an anti-TfR1 antibodies) described herein covalently linked to any of the molecular payloads (e.g., an oligonucleotide) described herein. In some embodiments, the muscle targeting agent (e.g., an anti-TfR1 antibody) is covalently linked to a molecular payload (e.g., an oligonucleotide) via a linker. Any of the linkers described herein may be used. In some embodiments, the linker is linked to the 5′ end, the 3′ end, or internally of the oligonucleotide. In some embodiments, the linker is linked to the antibody via a thiol-reactive linkage (e.g., via a cysteine in the antibody). In some embodiments, the linker is linked to the antibody (e.g., an anti-TfR1 antibody described herein) via an amine group (e.g., via a lysine in the antibody).

An example of a structure of a complex comprising an anti-TfR1 antibody covalently linked to an oligonucleotide via a linker is provided below:

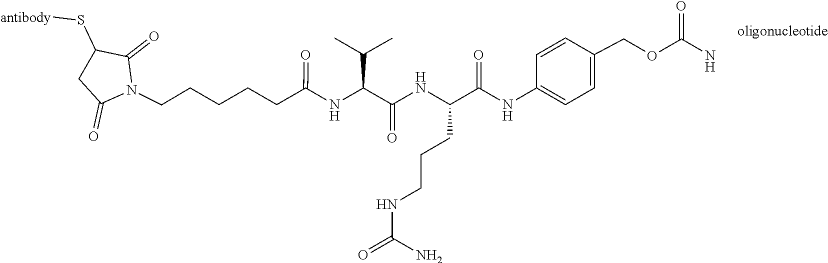

wherein the linker is linked to the 5′ end, the 3′ end, or internally of the oligonucleotide, and wherein the linker is linked to the antibody via a thiol-reactive linkage (e.g., via a cysteine in the antibody).

Another example of a structure of a complex comprising an anti-TfR1 antibody covalently linked to a molecular payload via a linker is provided below:

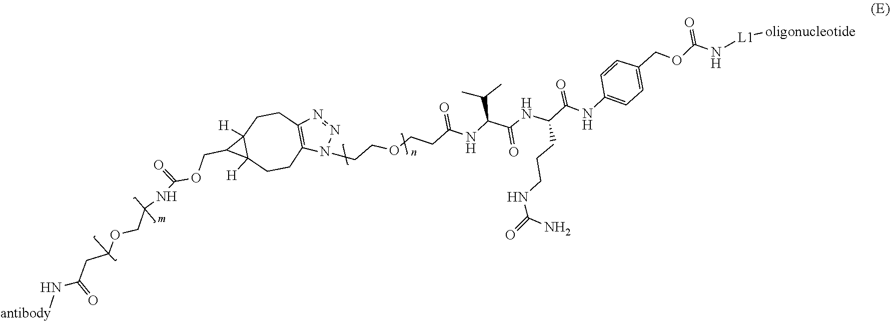

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine. In some embodiments, the oligonucleotide comprises a sense strand and an antisense strand, and the linker is linked to the sense strand or the antisense strand at the 5′ end or the 3′ end. In some embodiments, n is 3, and m is 4. In some embodiments, L1 is any one of the spacers described herein.

It should be appreciated that antibodies can be linked to oligonucleotides with different stoichiometries, a property that may be referred to as a drug to antibody ratios (DAR) with the “drug” being the oligonucleotide. In some embodiments, one oligonucleotide is linked to an antibody (DAR=1). In some embodiments, two oligonucleotides are linked to an antibody (DAR=2). In some embodiments, three oligonucleotides are linked to an antibody (DAR=3). In some embodiments, four oligonucleotides are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating oligonucleotides to different sites on an antibody and/or (e.g., and) by conjugating multimers to one or more sites on antibody. For example, a DAR of 2 may be achieved by conjugating a single oligonucleotide to two different sites on an antibody or by conjugating a dimer oligonucleotide to a single site of an antibody.

In some embodiments, the complex described herein comprises an anti-TfR1 antibody (e.g., an antibody or any variant thereof as described herein) covalently linked to an oligonucleotide. In some embodiments, the complex described herein comprises an anti-TfR1 antibody (e.g., an antibody or any variant thereof as described herein) covalently linked to an oligonucleotide via a linker. In some embodiments, the linker is linked to the 5′ end, the 3′ end, or internally of the oligonucleotide. In some embodiments, the oligonucleotide is a siRNA and the linker is linked to the 5′ end, the 3′ end, or internally of the sense strand of the siRNA. In some embodiments, the oligonucleotide is a siRNA and the linker is linked to the 5′ end, the 3′ end, or internally of the antisense strand of the siRNA. In some embodiments, the linker is linked to the antibody (e.g., an antibody or any variant thereof as described herein) via a thiol-reactive linkage (e.g., via a cysteine in the antibody). In some embodiments, the linker is linked to the antibody (e.g., an anti-TfR1 antibody described herein) via an amine group (e.g., via a lysine in the antibody).

In some embodiments, in any one of the examples of complexes described herein, the molecular payload is an oligonucleotide comprising a region of complementarity of at least 15 nucleotides (e.g., at least 15, at least 16, at least 17, at least 18, at least 19 or more) nucleotides to any one of the gene target sequences described herein, optionally wherein the target sequence is a sequence listed in Tables 8, 11, and 14. In some embodiments, in any one of the examples of complexes described herein, the molecular payload is an oligonucleotide comprising a region of complementarity of at least 15 nucleotides (e.g., at least 15, at least 16, at least 17, at least 18, at least 19 or more) nucleotides to any one of the gene target sequences described herein, optionally wherein the target sequence is a sequence listed in Tables 17, 20, 23, 26, 29, 32, 35, and 38.

In some embodiments, the complex described herein comprises an anti-TfR1 antibody covalently linked to a molecular payload (e.g., an oligonucleotide), wherein the anti-TfR1 antibody comprises a CDR-H1, a CDR-H2, and a CDR-H3 that are the same as the CDR-H1, CDR-H2, and CDR-H3 shown in Table 2, Table 6, and Table 7; and a CDR-L1, a CDR-L2, and a CDR-L3 that are the same as the CDR-L1, CDR-L2, and CDR-L3 shown in Table 2, Table 6, and Table 7.

In some embodiments, the complex described herein comprises an anti-TfR1 antibody covalently linked to a molecular payload (e.g., an oligonucleotide), wherein the anti-TfR1 antibody comprises:
(i) a CDR-H1 of SEQ ID NO: 1, a CDR-H2 of SEQ ID NO: 2, SEQ ID NO: 19, or SEQ ID NO: 23, a CDR-H3 of SEQ ID NO: 3, a CDR-L1 of SEQ ID NO: 4, a CDR-L2 of SEQ ID NO: 5, and a CDR-L3 of SEQ ID NO: 6;
(ii) a CDR-H1 of SEQ ID NO: 7, a CDR-H2 of SEQ ID NO: 8, SEQ ID NO: 20, or SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6; or
(iii) a CDR-H1 of SEQ ID NO: 12, a CDR-H2 of SEQ ID NO: 13, SEQ ID NO: 21, or SEQ ID NO: 25, a CDR-H3 of SEQ ID NO: 14, a CDR-L1 of SEQ ID NO: 15, a CDR-L2 of SEQ ID NO: 5, and a CDR-L3 of SEQ ID NO: 16. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 411). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises:
(i) a CDR-H1 of SEQ ID NO: 27, a CDR-H2 of SEQ ID NO: 28, a CDR-H3 of SEQ ID NO: 29, a CDR-L1 of SEQ ID NO: 30, a CDR-L2 of SEQ ID NO: 31, and a CDR-L3 of SEQ ID NO: 32;
(ii) a CDR-H1 of SEQ ID NO: 33, a CDR-H2 of SEQ ID NO: 34, a CDR-H3 of SEQ ID NO: 35, a CDR-L1 of SEQ ID NO: 36, a CDR-L2 of SEQ ID NO: 37, and a CDR-L3 of SEQ ID NO: 32; or
(iii) a CDR-H1 of SEQ ID NO: 38, a CDR-H2 of SEQ ID NO: 39, a CDR-H3 of SEQ ID NO: 40, a CDR-L1 of SEQ ID NO: 41, a CDR-L2 of SEQ ID NO: 31, and a CDR-L3 of SEQ ID NO: 42. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 411). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises:
(i) a CDR-H1 of SEQ ID NO: 45, SEQ ID NO: 63, or SEQ ID NO: 66, a CDR-H2 of SEQ ID NO: 46, a CDR-H3 of SEQ ID NO: 47, a CDR-L1 of SEQ ID NO: 48, a CDR-L2 of SEQ ID NO: 49, and a CDR-L3 of SEQ ID NO: 50;
(ii) a CDR-H1 of SEQ ID NO: 51, SEQ ID NO: 64, or SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(iii) a CDR-H1 of SEQ ID NO: 56, a CDR-H2 of SEQ ID NO: 57, a CDR-H3 of SEQ ID NO: 58, a CDR-L1 of SEQ ID NO: 59, a CDR-L2 of SEQ ID NO: 49, and a CDR-L3 of SEQ ID NO: 60. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 411). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises:
(i) a CDR-H1 of SEQ ID NO: 110, a CDR-H2 of SEQ ID NO: 111, a CDR-H3 of SEQ ID NO: 112, a CDR-L1 of SEQ ID NO: 113, a CDR-L2 of SEQ ID NO: 114, and a CDR-L3 of SEQ ID NO: 115;
(ii) a CDR-H1 of SEQ ID NO: 116, a CDR-H2 of SEQ ID NO: 117, a CDR-H3 of SEQ ID NO: 112, a CDR-L1 of SEQ ID NO: 113, a CDR-L2 of SEQ ID NO: 114, and a CDR-L3 of SEQ ID NO: 115; or
(iii) a CDR-H1 of SEQ ID NO: 118, a CDR-H2 of SEQ ID NO: 119, a CDR-H3 of SEQ ID NO: 120, a CDR-L1 of SEQ ID NO: 121, a CDR-L2 of SEQ ID NO: 122, and a CDR-L3 of SEQ ID NO: 123. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 152). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises VH as shown in Table 2 or Table 3; and a VL as shown in Table 2 or Table 3. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 152). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises a heavy chain and light chain of any one of the antibodies listed in Tables 4 and 5. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 152). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises a VH having the amino acid sequence of SEQ ID NO: 124 and a VL having the amino acid sequence of SEQ ID NO: 125. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 152). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked to a molecular payload (e.g., an oligonucleotide), wherein the anti-TfR1 antibody comprises a heavy chain having the amino acid sequence of any one of SEQ ID NOs: 132, 133, 136, and 137, and a light chain having the amino acid sequence of SEQ ID No: 133 or SEQ ID NO: 135. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 152). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 antibody covalently linked via a lysine to the 5′ end of an oligonucleotide, wherein the antibody is a Fab fragment of an IgG1 kappa that comprises human framework regions with the CDRs of a murine antibody listed in Table 2 (e.g., 3A4, 3M12, or 5H12), wherein the complex has the structure of:

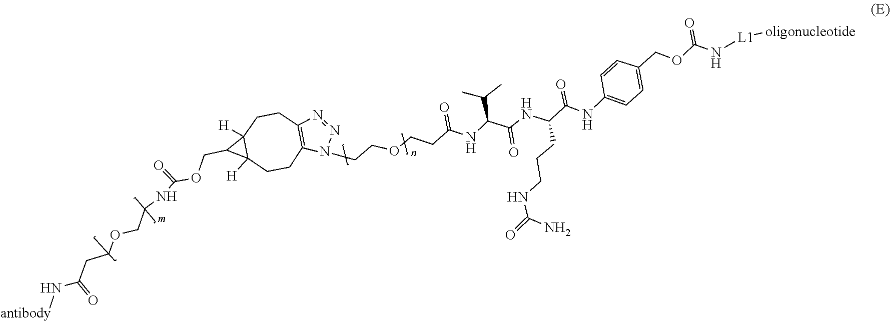

wherein n is 3 and m is 4. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the molecular payload is an oligonucleotide that is complementary to an MLCK1 mRNA molecule (e.g., the MLCK1 mRNA as set forth in SEQ ID NO: 152). In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40.

In some embodiments, the complex described herein comprises an anti-TfR1 Fab covalently linked via a lysine to an oligonucleotide (e.g., an oligonucleotide targeting MSTN, INHBA, ACVR1B, MLCK1, ACVR1, FBXO32, TRIM63, MEF2D, KLF15, MED1, MED13, or PPP1R3A), wherein the anti-TfR1 Fab comprises a CDR-H1, a CDR-H2, a CDR-H3, a CDR-L1, a CDR-L2, and a CDR-L3 of any one of the antibodies listed in Table 2, Table 6, and Table 7; wherein the complex has the structure of:

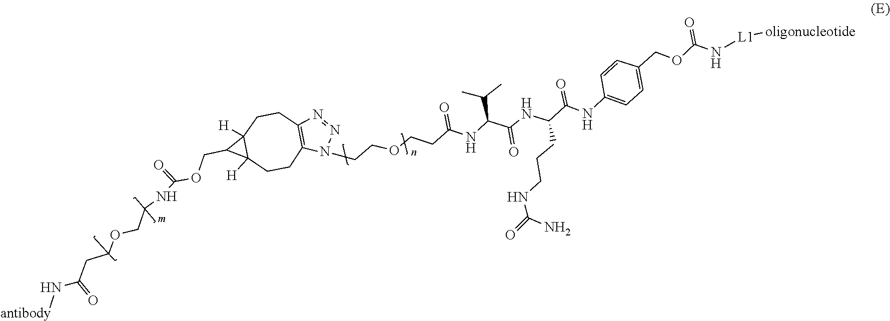

wherein n is 3 and m is 4. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the oligonucleotide in the complex is an siRNA with complementarity to an MLCK1 mRNA molecule which is linked at the 5′ end or 3′ end of the sense strand or the antisense strand. In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40. In some embodiments, the oligonucleotide in the complex is an siRNA listed in Table 10, Table 13, Table 16, Table 19, Table 22, Table 25, Table 28, Table 31, Table 34, Table 37, or Table 40 which is linked at the 5′ end or 3′ end of the sense strand or the antisense strand.

In some embodiments, the complex described herein comprises an anti-TfR1 Fab covalently linked via a lysine to an oligonucleotide (e.g., an oligonucleotide targeting MSTN, INHBA, ACVR1B, MLCK1, ACVR1, FBXO32, TRIM63, MEF2D, KLF15, MED1, MED13, or PPP1R3A), wherein the anti-TfR1 Fab comprises a VH and VL of any one of the antibodies listed in Table 2 or Table 3; wherein the complex has the structure of:

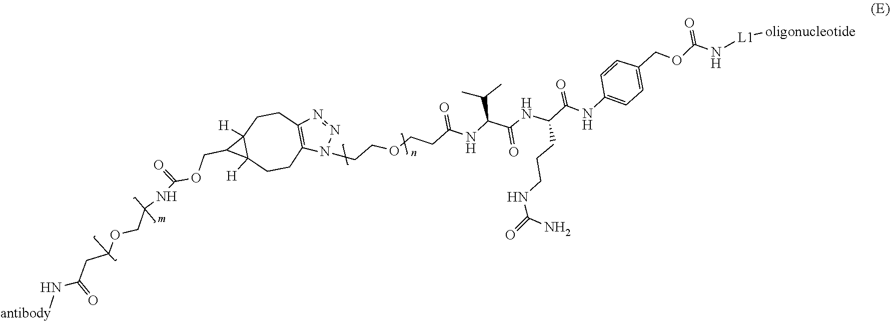

wherein n is 3 and m is 4. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the oligonucleotide in the complex is an siRNA with complementarity to an MLCK1 mRNA molecule which is linked at the 5′ end or 3′ end of the sense strand or the antisense strand. In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40. In some embodiments, the oligonucleotide in the complex is an siRNA listed in Table 10, Table 13, Table 16, Table 19, Table 22, Table 25, Table 28, Table 31, Table 34, Table 37, or Table 40 which is linked at the 5′ end or 3′ end of the sense strand or the antisense strand.

In some embodiments, the complex described herein comprises an anti-TfR1 Fab covalently linked via a lysine to an oligonucleotide (e.g., an oligonucleotide targeting MSTN, INHBA, ACVR1B, MLCK1, ACVR1, FBXO32, TRIM63, MEF2D, KLF15, MED1, MED13, PPP1R3A), wherein the anti-TfR1 Fab comprises a VH comprising the amino acid sequence of SEQ ID NO: 76 and a VL comprising the amino acid sequence of SEQ ID NO: 75; wherein the complex has the structure of:

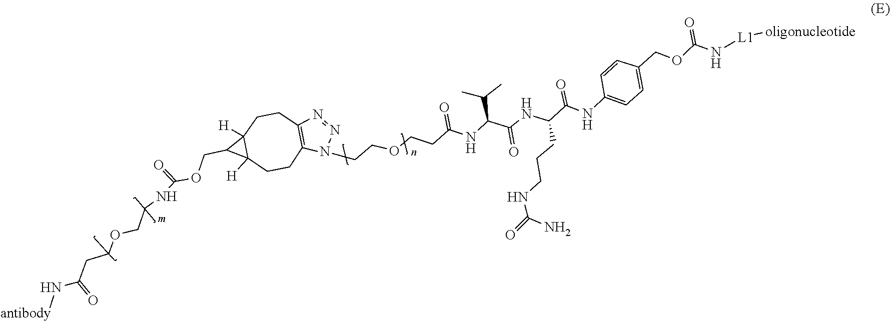

wherein n is 3 and m is 4. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the oligonucleotide in the complex is an siRNA with complementarity to an MLCK1 mRNA molecule which is linked at the 5′ end or 3′ end of the sense strand or the antisense strand. In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40. In some embodiments, the oligonucleotide in the complex is an siRNA listed in Table 10, Table 13, Table 16, Table 19, Table 22, Table 25, Table 28, Table 31, Table 34, Table 37, or Table 40 which is linked at the 5′ end or 3′ end of the sense strand or the antisense strand.

In some embodiments, the complex described herein comprises an anti-TfR1 Fab covalently linked via a lysine to an oligonucleotide (e.g., an oligonucleotide targeting MSTN, INHBA, ACVR1B, MLCK1, ACVR1, FBXO32, TRIM63, MEF2D, KLF15, MED1, MED13, or PPP1R3A), wherein the anti-TfR1 Fab comprises a heavy chain and light chain of any one of the antibodies listed in Table 5; wherein the complex has the structure of:

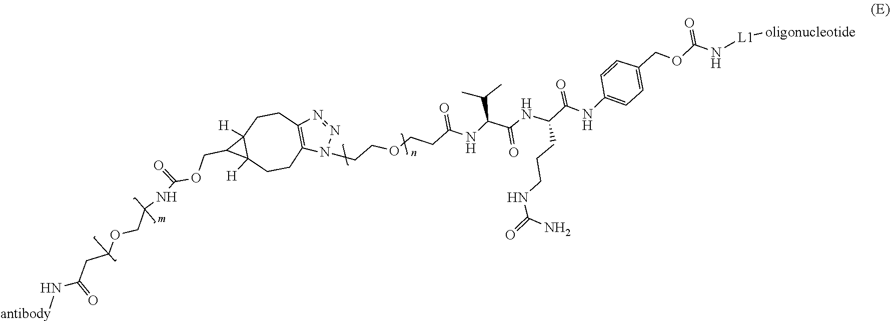

wherein n is 3 and m is 4. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the oligonucleotide in the complex is an siRNA with complementarity to an MLCK1 mRNA molecule which is linked at the 5′ end or 3′ end of the sense strand or the antisense strand. In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40. In some embodiments, the oligonucleotide in the complex is an siRNA listed in Table 10, Table 13, Table 16, Table 19, Table 22, Table 25, Table 28, Table 31, Table 34, Table 37, or Table 40 which is linked at the 5′ end or 3′ end of the sense strand or the antisense strand.

In some embodiments, the complex described herein comprises an anti-TfR1 Fab covalently linked via a lysine to an oligonucleotide (e.g., an oligonucleotide targeting MSTN, INHBA, ACVR1B, MLCK1, ACVR1, FBXO32, TRIM63, MEF2D, KLF15, MED1, MED13, or PPP1R3A), wherein the anti-TfR1 Fab comprises a heavy chain comprising the amino acid sequence of SEQ ID NO: 101 and a light chain comprising the amino acid sequence of SEQ ID NO: 90; wherein the complex has the structure of:

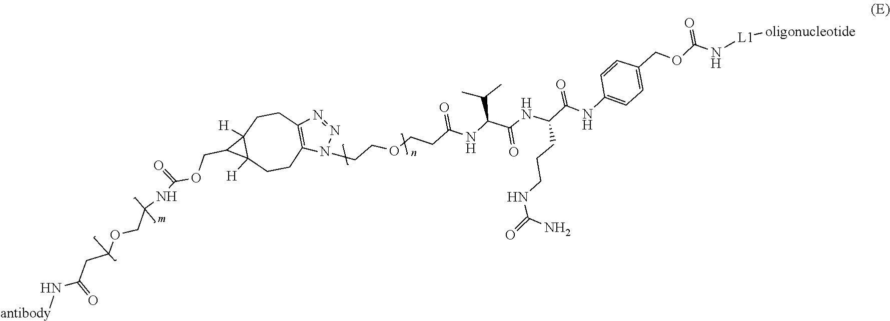

wherein n is 3 and m is 4. In some embodiments, the molecular payload is an MSTN targeting oligonucleotide comprising at least 16 nucleotides of a sequence listed in Table 9, optionally wherein the molecular payload is an MSTN targeting siRNA listed in Table 10. In some embodiments, the molecular payload is an INHBA targeting oligonucleotide comprising at least 16 nucleotides of a sequence listed in Table 12, optionally wherein the molecular payload is an INHBA targeting siRNA listed in Table 13. In some embodiments, the molecular payload is an ACVR1B targeting oligonucleotide comprising at least 16 nucleotides of a sequence listed in Table 15, optionally wherein the molecular payload is an ACVR1B targeting siRNA listed in Table 16. In some embodiments, the oligonucleotide in the complex is an siRNA with complementarity to an MLCK1 mRNA molecule which is linked at the 5′ end or 3′ end of the sense strand or the antisense strand. In some embodiments, the molecular payload is an ACVR1 targeting oligonucleotide comprising at least 16 nucleotides of a sequence listed in Table 18, optionally wherein the molecular payload is an ACVR1 targeting siRNA listed in Table 19. In some embodiments, the molecular payload is a FBXO32 targeting oligonucleotide comprising at least 16 nucleotides of a sequence listed in Table 21, optionally wherein the molecular payload is a FBXO32 targeting siRNA listed in Table 22. In some embodiments, the molecular payload is a TRIM63 targeting oligonucleotide comprising at least 16 nucleotides of a sequence listed in Table 24, optionally wherein the molecular payload is a TRIM63 targeting siRNA listed in Table 25. In some embodiments, the molecular payload is a MEF2D targeting oligonucleotide comprising at least 16 nucleotides of a sequence listed in Table 27, optionally wherein the molecular payload is a MEF2D targeting siRNA listed in Table 28. In some embodiments, the molecular payload is a KLF15 targeting oligonucleotide comprising at least 16 nucleotides of a sequence listed in Table 30, optionally wherein the molecular payload is a KLF15 targeting siRNA listed in Table 31. In some embodiments, the molecular payload is a MED1 targeting oligonucleotide comprising at least 16 nucleotides of a sequence listed in Table 33, optionally wherein the molecular payload is a MED1 targeting siRNA listed in Table 34. In some embodiments, the molecular payload is a MED13 targeting oligonucleotide comprising at least 16 nucleotides of a sequence listed in Table 36, optionally wherein the molecular payload is a MED13 targeting siRNA listed in Table 37. In some embodiments, the molecular payload is a PPP1R3A targeting oligonucleotide comprising at least 16 nucleotides of a sequence listed in Table 39, optionally wherein the molecular payload is a PPP1R3A targeting siRNA listed in Table 40. In some embodiments, the oligonucleotide in the complex is an siRNA listed in Table 10, Table 13, Table 16, Table 19, Table 22, Table 25, Table 28, Table 31, Table 34, Table 37, or Table 40 which is linked at the 5′ end or 3′ end of the sense strand or the antisense strand.

In some embodiments, in any one of the examples of complexes described herein, L1 is:

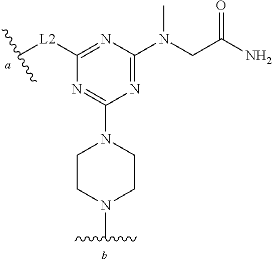

wherein L2 is

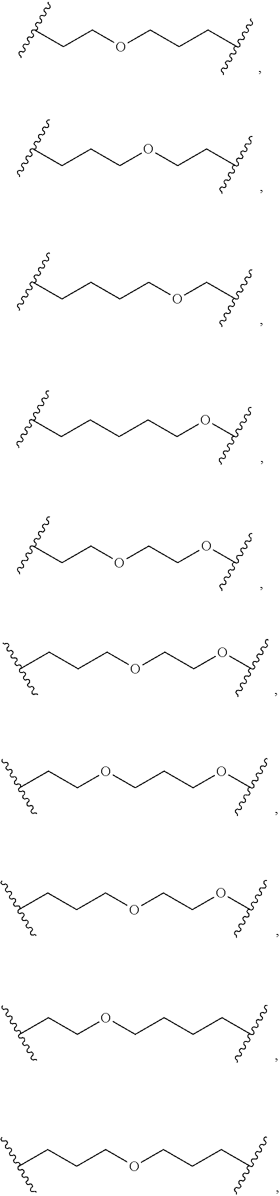

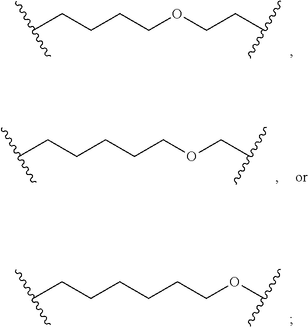 [SMILES: *CCCCOCC* ; *CCCCCOC* ; *CCCCCCO*]

wherein a labels the site directly linked to the carbamate moiety of formulae (B), (D), (E), and (I); and b labels the site covalently linked (directly or via additional chemical moieties) to the oligonucleotide.

In some embodiments, L1 is:

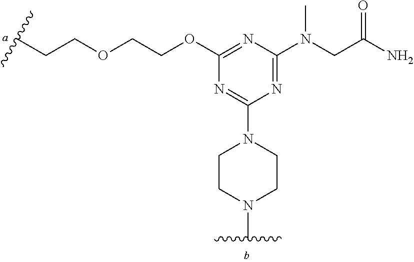 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

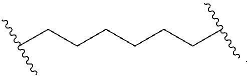 [SMILES: *CCCCCC*]

In some embodiments, L1 is linked to a 5′ phosphate of the oligonucleotide. In some embodiments, L1 is linked to a 5′ phosphorothioate of the oligonucleotide. In some embodiments, L1 is linked to a 5′ phosphonoamidate of the oligonucleotide.

In some embodiments, L1 is linked to a 5′ phosphate of the oligonucleotide. In some embodiments, the linkage of L1 to a 5′ phosphate of the oligonucleotide forms a phosphodiester bond between L1 and the oligonucleotide.

In some embodiments, L1 is linked to a 3′ phosphate of the oligonucleotide. In some embodiments, the linkage of L1 to a 3′ phosphate of the oligonucleotide forms a phosphodiester bond between L1 and the oligonucleotide.

In some embodiments, L1 is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

### MSTN, ACVR1B, INHBA

In some aspects, complexes comprising a muscle-targeting agent covalently linked to a molecular payload as described herein are effective in treating heart failure, muscle atrophy (e.g., skeletal and/or cardiac muscle atrophy), muscular dystrophies, cachexia (e.g., cardiac cachexia), muscle hypertrophy (e.g., cardiac hypertrophy), cardiac muscle wasting, cardiac fibrosis, and/or cardiomyopathy. In some embodiments, complexes as described herein are effective in treating myocardial complications (e.g., heart failure) in subjects having type 2 diabetes. In some embodiments, complexes are effective in treating any disease or condition that involves a thickening of the heart and/or an increase in extracellular matrix in the heart. In some embodiments, cardiac fibrosis or cardiac hypertrophy is associated with an increased level or angiotensin-II.

In some embodiments, complexes are effective in specifically targeting expression of MSTN and/or INHBA in cardiac cells. In some embodiments, complexes are effective in treating and/or preventing heart failure (e.g., involving cardiac muscle wasting, cardio myopathy, or cachexia). Heart failure is typically characterized by diverse metabolic disturbances, many of which adversely affect muscle and fat metabolism, thereby leading to cachexia. In particular, skeletal muscle atrophy is prevalent in chronic heart failure patients. In some embodiments, heart failure is associated with cardiac muscle and/or skeletal muscle wasting. In some embodiments, the heart failure is associated with cardiomyopathy, which refers to a group of diseases of the heart muscle that makes it more difficult for the heart to pump blood to the rest of the body. In some embodiments, the cardiomyopathy is dilated cardiomyopathy, in which the pumping ability of the left ventricle becomes enlarged (dilated) and decreases the effectiveness of pumping out blood. In some embodiments, the cardiomyopathy is hypertrophic cardiomyopathy, which involves abnormal thickening of the heart muscle. In some embodiments, the cardiomyopathy is restrictive cardiomyopathy, in which the heart muscle becomes rigid and less elastic. In some embodiments, the cardiomyopathy is arrhythmogenic right ventricular dysplasia in which the muscle of the right ventricle is replaced by scar tissue.

In some embodiments, the heart failure is associated with atrophy of the heart. Atrophy of the heart refers to the acquired reduction in the size and mass of the heart. In some embodiments, the atrophy is concentric atrophy in which the cavity is diminished in size, but the wall remains the same. In some embodiments, the atrophy is aneurysmal atrophy in which the walls are thinned and the heart chambers dilated. In some embodiments, the atrophy is simple type atrophy in which the muscular walls are thinned with little change in the volume of the heart. In some embodiments, the heart failure is associated with a decrease in cardiac muscle mass. In some embodiments, the heart failure is associated with a 5%, 10%, 20%, 30%, 40%, 50%, 60%, 70%, 75%, 80%, or more decrease in cardiac muscle mass.

In some embodiments, heart failure is associated with a decrease in heart function (e.g., ejection fraction). Ejection fraction is a measurement, expressed as a percentage, of how much blood the left ventricle pumps out with each contraction. In some embodiments, a typical ejection fraction is from 50% to 70%. In some embodiments, the heart failure is associated with a 5%, 10%, 20%, 30%, 40%, 50%, 60%, 70%, 75%, 80%, 85%, 90%, 95%, or greater decrease in heart function (e.g., ejection fraction or volume of blood per pump). In some embodiments, the heart failure is associated with an ejection fraction of 50%, 45%, 40%, 35%, 30%, 25%, 20%, 15%, 10%, 5%, or lower. It should be appreciated that ejection fraction is not the only measure of heart function and this disclosure is not meant to be limiting in that respect. For example, in some embodiments, the measure of heart function may be based on a measurement of the volume of blood (e.g., per pump) that the heart pumps out.

In some embodiments, the heart failure is associated with cardiac cachexia. The compositions and methods provided herein may be used to treat or prevent a subject having or at risk of developing cardiac cachexia. Cardiac cachexia is characterized, inter alia, by severe weight loss related to heart disease. Cardiac cachexia may be characterized on the basis of the presence of unintentional and non-edematous weight loss (e.g., greater than 5%, 6%, 7%, 8%, 9%, 10%, 15% or greater) of a premorbid normal weight of an individual.

One way to treat or prevent heart failure is to inhibit the negative muscle regulator, myostatin, in cardiac and/or skeletal muscle cells.

In some embodiments, a subject may be a human subject, a non-human primate subject, a rodent subject, or any suitable mammalian subject. In some embodiments, a subject may have myotonic dystrophy.

In some embodiments, a subject having muscle hypertrophy has at least one mutation in MSTN as in Schuelke, M. et al., “Myostatin Mutation Associated with Gross Muscle Hypertrophy in a Child” N Engl J Med 2004; 350:2682-2688.

In some embodiments, complexes are effective in targeting activity of MSTN and/or INHBA in any muscle tissue (e.g., cardiac muscle, skeletal muscle). In some embodiments, complexes that target activity of MSTN or INHBA in skeletal muscle tissues are effective at treating a subject having skeletal muscle atrophy (e.g., resulting from overexpressed and hyperactive MSTN or INHBA).

In some embodiments, a subject is administered complexes targeting MSTN and complexes targeting ACVR1B. In some embodiments, such administration leads to increased muscle size and function.

In some embodiments, a subject has a thickening of the heart and/or an increase in extracellular matrix in the heart. In some embodiments, a subject has and/or is suffering from cardiac fibrosis or cardiac hypertrophy. In some embodiments, a subject has and/or is suffering from angiotensin-II induced cardiac hypertrophy. In some embodiments, a subject has recently experienced a cardiac infarction (i.e., heart attack).

Cardiomyopathy is a disease of the heart muscle that makes it harder for your heart to pump blood to the rest of your body. Cardiomyopathy can lead to heart failure. The main types of cardiomyopathy include dilated, hypertrophic and restrictive cardiomyopathy.

Cardiac hypertrophy is generally characterized by atypical increase in size or thickening of the heart, resulting from atypical increase in the size of cardiomyocytes and other atypical developments in the heart, such as increased thickening of the extracellular matrix. In some embodiments, complexes are effective in reducing the size (e.g., muscle mass) or thickening of the heart of a subject having cardiac hypertrophy (e.g., by at least 5%, 10%, 20%, 30%, 40%, or 50%, relative to a control subject or baseline measurement). In some embodiments, complexes are effective in slowing the increase in size or thickening of the heart of a subject having cardiac hypertrophy (e.g., slow the rate of increase by at least 5%, 10%, 20%, 30%, 40%, or 50%, relative to a control subject or baseline rate).

In some embodiments, a cardiac hypertrophy is angiotensin II-induced cardiac hypertrophy. Angiotensin II, a common medication used to treat hypotension, has been shown to induce cardiac hypertrophy in selected patient subjects. Angiotensin II can induce cardiac hypertrophy indirectly (e.g., resulting from it vasoconstrictive effects) and/or directly (e.g., resulting from its cardiac trophic effects). In some embodiments, a subject having angiotensin II-induced cardiac hypertrophy has not previously experienced cardiac hypertrophy.

In some embodiments, the subject that has or is suspected of having impaired muscle and cardiac development has an increased level of ACVR1B expression and/or activity (e.g., increased by at least 20%, at least 30%, at least 40%, at least 50%, at least 60%, at least 70%, at least 80%, at least 90%, or more), compared to the ACVR1B expression and/or activity level in a healthy subject. In some embodiments, a complex comprising a muscle-targeting agent covalently linked to a molecular payload as described herein is effective in decreasing the ACVR1B expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in decreasing the ACVR1B expression and/or activity to the level of a healthy subject.

In some embodiments, the subject that has or is suspected of having a heart disease (e.g., cardiac hypertrophy, cardiomyopathy) has an increased level of ACVR1B expression and/or activity (e.g., increased by at least 20%, at least 30%, at least 40%, at least 50%, at least 60%, at least 70%, at least 80%, at least 90%, or more), compared to the ACVR1B expression and/or activity level in a healthy subject. In some embodiments, a complex comprising a muscle-targeting agent covalently linked to a molecular payload as described herein is effective in reducing the ACVR1B expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in reducing the ACVR1B expression and/or activity to the level of a healthy subject. In some embodiments, complexes are effective in specifically targeting expression of ACVR1B in cardiac cells.

In some embodiments, a pharmaceutical composition may comprise more than one complex comprising a muscle-targeting agent covalently linked to a molecular payload. As a non-limiting example, a pharmaceutical composition may comprise two or more complexes each comprising a muscle-targeting agent linked to one of MSTN, INHBA and ACVR1B. In some embodiments, a pharmaceutical composition may comprise one complex comprising a muscle targeting agent linked to a molecular payload targeting MSTN, and a second complex comprising a muscle-targeting agent linked to a molecular payload targeting ACVR1B. In some embodiments, a pharmaceutical composition may comprise one complex comprising a muscle targeting agent linked to a molecular payload targeting MSTN, and a second complex comprising a muscle-targeting agent linked to a molecular payload targeting INHBA. In some embodiments, a pharmaceutical composition may comprise one complex comprising a muscle targeting agent linked to a molecular payload targeting INHBA, and a second complex comprising a muscle-targeting agent linked to a molecular payload targeting ACVR1B. In some embodiments, treatment of a subject with complexes targeted to two or more of MSTN, INHBA and ACVR1B simultaneously leads to improved outcomes, such as increased muscle size and function, relative to treatment with a single complex targeting only one of MSTN, INHBA and ACVR1B. In some embodiments, a pharmaceutical composition may further comprise any other suitable therapeutic agent for treatment of a subject, e.g., a human subject having heart failure, muscle atrophy (including but not limited to cardiac muscle atrophy), muscular dystrophies, cachexia, cardiac fibrosis, and/or muscle hypertrophy (including but not limited to cardiac hypertrophy). In some embodiments, the other therapeutic agents may enhance or supplement the effectiveness of the complexes described herein. In some embodiments, the other therapeutic agents may function to treat a different symptom or disease than the complexes described herein.

### MLCK1

In some aspects, complexes comprising a muscle-targeting agent covalently linked to a molecular payload as described herein are effective in treating irritable bowel syndrome. In some embodiments, complexes are effective in treating patients having inflammatory bowel disease. In some embodiments, a patient having inflammatory bowel disease also suffers from irritable bowel syndrome.

In some embodiments, complexes are effective in treating patients having familial thoracic aortic aneurysms and dissections (FTAAD). In some embodiments, FTAAD is associated with a genetic mutation of MLCK1 as is described in Hannuksela M. et al. “A novel variant in MYLK causes thoracic aortic dissections: genotypic and phenotypic description.” BMC Med Genet. 2016 Sep. 1; 17(1):61; and Shalata, A. et al. “Fatal thoracic aortic aneurysm and dissection in a large family with a novel MYLK gene mutation: delineation of the clinical phenotype.” Orphanet J Rare Dis. 2018 Mar. 15; 13(1):41.

In some embodiments, complexes are effective in treating patients having Berdon syndrome (recessive megacystis microcolon intestinal hypoperistalsis syndrome). In some embodiments, Berdon syndrome is associated with a genetic mutation of MLCK1 as is described in Halim D. et al. “Loss-of-Function Variants in MYLK Cause Recessive Megacystis Microcolon Intestinal Hypoperistalsis Syndrome.” Am J Hum Genet. 2017 Jul. 6; 101(1):123-129.

In some embodiments, a subject may be a human subject, a non-human primate subject, a rodent subject, or any suitable mammalian subject.

Irritable bowel syndrome (IBS) is characterized by painful defecation, irregular stool frequency, poor stool consistency, and/or epithelial barrier dysfunction. IBS is known to occur in patients having inflammatory bowel disease who are in remission from symptoms. Symptoms of IBS can include recurrent abdominal pain or discomfort (e.g., recurrent pain or discomfort for at least 3 days per month; or at least 1 day per week in the previous 3 months), at least 25% of defecations comprise loose stools and/or at least 25% of defecations comprise irregularly hard stools.

Inflammatory bowel disease (IBD) refers to chronic conditions that cause inflammation in some part of the intestines. The intestinal walls become swollen, inflamed, and develop ulcers, which can cause discomfort and serious digestive problems. Crohn's disease is a form of IBD that can happen anywhere along the digestive tract. It affects the deeper layers of the digestive lining and can show up as “skip lesions” between healthy areas. IBD includes Crohn's disease and Ulcerative colitis. Crohn's disease often involves the small intestine, the colon, or both. Internal tissues may develop shallow, crater-like areas or deeper sores and a cobblestone pattern. Ulcerative colitis involves only the colon and rectum. Inflammation and ulcers usually affect only the lining in these areas, compared with the deeper lesions seen in Crohn's disease.

In some embodiments, complexes are effective in reducing inflammation associated with IBD. In some embodiments, complexes are effective in reducing inflammation by at least 5%, 10%, 20%, 30%, 40%, or 50%, relative to a control subject or baseline measurement.

In some embodiments, complexes are effective in reducing the frequency of abdominal pain or discomfort. In some embodiments, complexes are effective in reducing the frequency of abdominal pain or discomfort by at least 5%, 10%, 20%, 30%, 40%, or 50%, relative to a control subject or baseline measurement. In some embodiments, complexes are effective in reducing the frequency of abdominal pain or discomfort to fewer than 3, fewer than 2, or fewer than 1 day of discomfort per month.

In some embodiments, complexes are effective in reducing the frequency of defecations that comprise irregularly hard stools. In some embodiments, complexes are effective in reducing the frequency of defecations that comprise irregularly hard stools by at least 5%, 10%, 20%, 30%, 40%, or 50%, relative to a control subject or baseline measurement. In some embodiments, complexes are effective in reducing the frequency of defecations that comprise irregularly hard stools to fewer than 25%, fewer than 20%, fewer than 15%, fewer than 10%, or fewer than 5% of all defecations.

In some embodiments, complexes are effective in reducing the frequency of defecations that comprise loose stools. In some embodiments, complexes are effective in reducing the frequency of defecations that comprise loose stools by at least 5%, 10%, 20%, 30%, 40%, or 50%, relative to a control subject or baseline measurement. In some embodiments, complexes are effective in reducing the frequency of defecations that comprise loose stools to fewer than 25%, fewer than 20%, fewer than 15%, fewer than 10%, or fewer than 5% of all defecations.

### ACVR1

In some aspects, complexes comprising a muscle-targeting agent covalently linked to a molecular payload as described herein are effective in treating cardiac hypertrophy. In some embodiments, complexes are effective in treating angiotensin II-induced cardiac hypertrophy. In some embodiments, cardiac hypertrophy can lead to heart failure.

In some embodiments, complexes are effective in specifically targeting expression of ACVR1 in cardiac cells. In some embodiments, complexes are effective in treating and/or preventing cardiac hypertrophy (e.g., angiotensin IT-induced cardiac hypertrophy). Cardiac hypertrophy is generally characterized by atypical increase in size or thickening of the heart, resulting from atypical increase in the size of cardiomyocytes and other atypical developments in the heart, such as increased thickening of the extracellular matrix. In some embodiments, complexes are effective in reducing the size (e.g., muscle mass) or thickening of the heart of a subject having cardiac hypertrophy (e.g., by at least 5%, 10%, 20%, 30%, 40%, or 50%, relative to a control subject or baseline measurement). In some embodiments, complexes are effective in slowing the increase in size or thickening of the heart of a subject having cardiac hypertrophy (e.g., slow the rate of increase by at least 5%, 10%, 20%, 30%, 40%, or 50%, relative to a control subject or baseline rate).

In some embodiments, a cardiac hypertrophy is angiotensin II-induced cardiac hypertrophy. Angiotensin II, a common medication used to treat hypotension, has been shown to induce cardiac hypertrophy in selected patient subjects. Angiotensin II can induce cardiac hypertrophy indirectly (e.g., resulting from its vasoconstrictive effects) and/or directly (e.g., resulting from its cardiac trophic effects). In some embodiments, a subject having angiotensin II-induced cardiac hypertrophy has not previously experienced cardiac hypertrophy.

Heart failure is typically characterized by diverse metabolic disturbances, many of which adversely affect muscle and fat metabolism, thereby leading to cachexia. In particular, skeletal muscle atrophy is prevalent in chronic heart failure patients. In some embodiments, heart failure is associated with cardiac muscle and/or skeletal muscle wasting. In some embodiments, the heart failure is associated with cardiomyopathy, which refers to a group of diseases of the heart muscle that makes it more difficult for the heart to pump blood to the rest of the body. In some embodiments, the cardiomyopathy is dilated cardiomyopathy, in which the pumping ability of the left ventricle becomes enlarged (dilated) and decreases the effectiveness of pumping out blood. In some embodiments, the cardiomyopathy is arrhythmogenic right ventricular dysplasia in which the muscle of the right ventricle is replaced by scar tissue.

In some embodiments, heart failure is associated with a decrease in heart function (e.g., ejection fraction). Ejection fraction is a measurement, expressed as a percentage, of how much blood the left ventricle pumps out with each contraction. In some embodiments, a typical ejection fraction is from 50% to 70%. In some embodiments, the heart failure is associated with a 5%, 10%, 20%, 30%, 40%, 50%, 60%, 70%, 75%, 80%, 85%, 90%, 95%, or greater decrease in heart function (e.g., ejection fraction or volume of blood per pump). In some embodiments, the heart failure is associated with an ejection fraction of 50%, 45%, 40%, 35%, 30%, 25%, 20%, 15%, 10%, 5%, or lower. It should be appreciated that ejection fraction is not the only measure of heart function and this disclosure is not meant to be limiting in that respect. For example, in some embodiments, the measure of heart function may be based on a measurement of the volume of blood (e.g., per pump) that the heart pumps out.

In some embodiments, the heart failure is associated with muscle atrophy (e.g., sarcopenia or cachexia). In some embodiments, the subject has cardiac cachexia. The compositions and methods provided herein may be used to treat or prevent a subject having or at risk of developing cardiac cachexia. Cardiac cachexia is characterized, inter alia, by severe weight loss related to heart disease. Cardiac cachexia may be characterized on the basis of the presence of nonintentional and nonedematous weight loss (e.g., greater than 5%, 6%, 7%, 8%, 9%, 10%, 15% or greater) of a premorbid normal weight of an individual.

Complexes comprising a muscle-targeting agent covalently linked to a molecular payload as described herein are effective in treating FOP. In some embodiments, complexes are effective in treating typical FOP or atypical FOP. In some embodiments, FOP is associated with mutations in the ACVR1 gene that lead to mutations in ACVR1 protein, e.g., L196P, R202I, R206H, Q207E, G328R, G328W, G328E, G356D, R375P, AP197-F198.

In some embodiments, a subject may be a human subject, a non-human primate subject, a rodent subject, or any suitable mammalian subject. In some embodiments, a subject may have FOP. In some embodiments, a subject having FOP has at least one mutation in the ACRV1 gene that leads to a mutation in ACVR1 protein, e.g., L196P, R202I, R206H, Q207E, G328R, G328W, G328E, G356D, R375P, AP197-F198. In some embodiments, a subject having FOP has progressive ossification of muscle tissues, i.e., gradual replacement of muscle tissue with bone. In some embodiments, a subject having FOP has restricted movement, loss of mobility, and/or difficulties with breathing and eating.

In some embodiments, complexes are effective in targeting activity of ACVR1 in any muscle tissue (e.g., cardiac muscle, skeletal muscle). In some embodiments, complexes are effective in treating muscle atrophy.

In some embodiments, a pharmaceutical composition may comprise more than one complex comprising a muscle-targeting agent covalently linked to a molecular payload. In some embodiments, a pharmaceutical composition may further comprise any other suitable therapeutic agent for treatment of a subject, e.g., a human subject having cardiac hypertrophy and/or FOP. In some embodiments, the other therapeutic agents may enhance or supplement the effectiveness of the complexes described herein. In some embodiments, the other therapeutic agents may function to treat a different symptom or disease than the complexes described herein.

### FBXO32, TRIM63

In some aspects, complexes comprising a muscle-targeting agent covalently linked to a molecular payload as described herein are effective in treating muscle atrophy (e.g., sarcopenia or cachexia). Muscle atrophy may be caused by any condition. In some embodiments, complexes are effective in treating muscle atrophy caused by cancer cachexia, cardiac cachexia, denervation, glucocorticoid use, inactivity (muscle disuse), abnormal levels of myostatin, and/or fasting. In some embodiments, muscle atrophy is caused by cancer cachexia, fasting, renal failure, diabetes, denervation, or glucocorticoid-induced muscle atrophy.

In some embodiments, complexes are effective in specifically targeting expression of FBXO32 or TRIM63 in cardiac cells. In some embodiments, complexes are effective in treating and/or preventing heart failure (e.g., involving cardiac muscle wasting, cardio myopathy, or cachexia). Heart failure is typically characterized by diverse metabolic disturbances, many of which adversely affect muscle and fat metabolism, thereby leading to cachexia. In particular, skeletal muscle atrophy is prevalent in chronic heart failure patients. In some embodiments, heart failure is associated with cardiac muscle and/or skeletal muscle wasting. In some embodiments, the heart failure is associated with cardiomyopathy, which refers to a group of diseases of the heart muscle that makes it more difficult for the heart to pump blood to the rest of the body. In some embodiments, the cardiomyopathy is dilated cardiomyopathy, in which the pumping ability of the left ventricle becomes enlarged (dilated) and decreases the effectiveness of pumping out blood. In some embodiments, the cardiomyopathy is hypertrophic cardiomyopathy, which involves abnormal thickening of the heart muscle. In some embodiments, the cardiomyopathy is restrictive cardiomyopathy, in which the heart muscle becomes rigid and less elastic. In some embodiments, the cardiomyopathy is arrhythmogenic right ventricular dysplasia in which the muscle of the right ventricle is replaced by scar tissue.

In some embodiments, the heart failure is associated with atrophy of the heart. Atrophy of the heart refers to the acquired reduction in the size and mass of the heart. In some embodiments, the atrophy is concentric atrophy in which the cavity is diminished in size, but the wall remains the same. In some embodiments, the atrophy is aneurysmal atrophy in which the walls are thinned and the heart chambers dilated. In some embodiments, the atrophy is simple type atrophy in which the muscular walls are thinned with little change in the volume of the heart. In some embodiments, the heart failure is associated with a decrease in cardiac muscle mass. In some embodiments, the heart failure is associated with a 5%, 10%, 20%, 30%, 40%, 50%, 60%, 70%, 75%, 80%, or more decrease in cardiac muscle mass.

In some embodiments, heart failure is associated with a decrease in heart function (e.g., ejection fraction). Ejection fraction is a measurement, expressed as a percentage, of how much blood the left ventricle pumps out with each contraction. In some embodiments, a typical ejection fraction is from 50% to 70%. In some embodiments, the heart failure is associated with a 5%, 10%, 20%, 30%, 40%, 50%, 60%, 70%, 75%, 80%, 85%, 90%, 95%, or greater decrease in heart function (e.g., ejection fraction or volume of blood per pump). In some embodiments, the heart failure is associated with an ejection fraction of 50%, 45%, 40%, 35%, 30%, 25%, 20%, 15%, 10%, 5%, or lower. It should be appreciated that ejection fraction is not the only measure of heart function and this disclosure is not meant to be limiting in that respect. For example, in some embodiments, the measure of heart function may be based on a measurement of the volume of blood (e.g., per pump) that the heart pumps out.

In some embodiments, the heart failure is associated with cardiac cachexia. The compositions and methods provided herein may be used to treat or prevent a subject having or at risk of developing cardiac cachexia. Cardiac cachexia is characterized, inter alia, by severe weight loss related to heart disease. Cardiac cachexia may be characterized on the basis of the presence of nonintentional and nonedematous weight loss (e.g., greater than 5%, 6%, 7%, 8%, 9%, 10%, 15% or greater) of a premorbid normal weight of an individual.

In some embodiments, a subject may be a human subject, a non-human primate subject, a rodent subject, or any suitable mammalian subject. In some embodiments, a subject has increased levels of FBXO32 or TRIM63 as compared to a control.

In some embodiments, the subject has cancer cachexia, cardiac cachexia, denervation, glucocorticoid use, diabetes, renal failure, inactivity (e.g., muscle disuse), elevated levels of myostatin, and/or is fasting. In some embodiments, the subject has reduced muscle mass, reduced muscle size and/or reduced number of muscle cells as compared to a control. In some embodiments, a control is a healthy subject. As a non-limiting example, ultrasounds and/or magnetic resonance imaging (MRI) may be used to diagnose a subject with muscle atrophy. In some embodiments, muscle atrophy affects slow type 1 or fast type 2 muscle fibers in a subject. See, e.g., Talbot et al., Wiley Interdiscip Rev Dev Biol. 2016 July; 5(4):518-34, which is herein incorporated by reference in its entirety. In some embodiments, the subject does not have peripheral artery disease. In some embodiments, the peripheral arterial system of the subject is intact. In some instances, the muscle atrophy may not be localized to the subject's lower or hind limb extremities.

In some embodiments, complexes comprising a muscle-targeting agent covalently linked to a molecular payload (e.g., a mRNA encoding FBXO32 or a FBXO32 polypeptide) are effective in treating cardiac hypertrophy. In some embodiments, a subject that has cardiac hypertrophy has a reduced FBXO32 expression level in heart muscle cells (e.g., reduced by at least 10%, at least 20%, at least 30%, at least 40%, at least 50%, at least 60%, at least 70%, at least 80%, at least 90%, at least 95%, at least 99%, or more), compared to a healthy subject. In some embodiments, the complexes comprising a muscle-targeting agent covalently linked to a molecular payload (e.g., a mRNA encoding FBXO32 or a FBXO32 polypeptide as described herein increases FBXO32 expression level in heart muscle cells by at least 20%, at least 30%, at least 40%, at least 50%, at least 60%, at least 70%, at least 80%, at least 90%, at least 95%, at least 2-fold, at least 5-fold, at least 10-fold, at least 50-fold, at least 100-fold, or more), compared to without the complexes.

### MEF2D, KLF15, MED1, MED13, or PPP1R3A

In some aspects, complexes comprising a muscle-targeting agent covalently linked to a molecular payload as described herein are effective in treating subjects having impaired muscle and cardiac development (e.g. congenital heart defect), a muscular disease (e.g., muscle atrophy, myotonic dystrophy, cachexia), a heart disease (e.g., cardiac atrophy, cardiac hypertrophy, cardiomyopathy, cardiovascular disease, congenital heart disease, heart arrhythmias), heart failure, or a neuromuscular disease (e.g., Parkinson's disease, amyotrophic lateral sclerosis (ALS)). In some embodiments, the subject has coronary artery disease or high blood pressure. In some embodiments, the subject has a heart valve abnormality. In some embodiments, the subject has a heart murmur that is expected to lead to heart failure. In some embodiments, the subject has heart failure at Stage A, B, C, or D. In some embodiments, the subject has or is at risk of having congestive heart failure.

In some embodiments, a subject may be a human subject, a non-human primate subject, a rodent subject, or any suitable mammalian subject. In some embodiments, a subject may have myotonic dystrophy. In some embodiments, a subject has experienced a myocardial infarction (e.g., at least one myocardial infarction). In some embodiments, a subject has experienced 2, 3, 4, 5, 6, or more myocardial infarctions. In some embodiments, a subject is at an elevated risk of myocardial infarction.

Cardiomyopathy is a disease of the heart muscle that makes it harder for your heart to pump blood to the rest of your body. Cardiomyopathy can lead to heart failure. The main types of cardiomyopathy include dilated, hypertrophic and restrictive cardiomyopathy.

Cardiac hypertrophy is generally characterized by atypical increase in size or thickening of the heart, resulting from atypical increase in the size of cardiomyocytes and other atypical developments in the heart, such as increased thickening of the extracellular matrix. In some embodiments, complexes are effective in reducing the size (e.g., muscle mass) or thickening of the heart of a subject having cardiac hypertrophy (e.g., by at least 5%, 10%, 20%, 30%, 40%, or 50%, relative to a control subject or baseline measurement). In some embodiments, complexes are effective in slowing the increase in size or thickening of the heart of a subject having cardiac hypertrophy (e.g., slow the rate of increase by at least 5%, 10%, 20%, 30%, 40%, or 50%, relative to a control subject or baseline rate).

In some embodiments, a cardiac hypertrophy is angiotensin II-induced cardiac hypertrophy. Angiotensin II, a common medication used to treat hypotension, has been shown to induce cardiac hypertrophy in selected patient subjects. Angiotension II can induce cardiac hypertrophy indirectly (e.g., resulting from its vasoconstrictive effects) and/or directly (e.g., resulting from its cardiac trophic effects). In some embodiments, a subject having angiotensin II-induced cardiac hypertrophy has not previously experienced cardiac hypertrophy.

Heart failure is typically characterized by diverse metabolic disturbances, many of which adversely affect muscle and fat metabolism, thereby leading to cachexia. In particular, skeletal muscle atrophy is prevalent in chronic heart failure patients. In some embodiments, heart failure is associated with cardiac muscle and/or skeletal muscle wasting. In some embodiments, the heart failure is associated with cardiomyopathy, which refers to a group of diseases of the heart muscle that makes it more difficult for the heart to pump blood to the rest of the body. In some embodiments, the cardiomyopathy is dilated cardiomyopathy, in which the pumping ability of the left ventricle becomes enlarged (dilated) and decreases the effectiveness of pumping out blood. In some embodiments, the cardiomyopathy is arrhythmogenic right ventricular dysplasia in which the muscle of the right ventricle is replaced by scar tissue.

In some embodiments, heart failure is associated with a decrease in heart function (e.g., ejection fraction). Ejection fraction is a measurement, expressed as a percentage, of how much blood the left ventricle pumps out with each contraction. In some embodiments, a typical ejection fraction is from 50% to 70%. In some embodiments, the heart failure is associated with a 5%, 10%, 20%, 30%, 40%, 50%, 60%, 70%, 75%, 80%, 85%, 90%, 95%, or greater decrease in heart function (e.g., ejection fraction or volume of blood per pump). In some embodiments, the heart failure is associated with an ejection fraction of 50%, 45%, 40%, 35%, 30%, 25%, 20%, 15%, 10%, 5%, or lower. It should be appreciated that ejection fraction is not the only measure of heart function and this disclosure is not meant to be limiting in that respect. For example, in some embodiments, the measure of heart function may be based on a measurement of the volume of blood (e.g., per pump) that the heart pumps out.

In some embodiments, the heart failure is associated with cardiac cachexia. The compositions and methods provided herein may be used to treat or prevent a subject having or at risk of developing cardiac cachexia. Cardiac cachexia is characterized, inter alia, by severe weight loss related to heart disease. Cardiac cachexia may be characterized on the basis of the presence of nonintentional and nonedematous weight loss (e.g., greater than 5%, 6%, 7%, 8%, 9%, 10%, 15% or greater) of a premorbid normal weight of an individual.

In some embodiments, the heart failure is associated with atrophy of the heart. Atrophy of the heart refers to the acquired reduction in the size and mass of the heart. In some embodiments, the atrophy is concentric atrophy in which the cavity is diminished in size, but the wall remains the same. In some embodiments, the atrophy is aneurysmal atrophy in which the walls are thinned and the heart chambers dilated. In some embodiments, the atrophy is simple type atrophy in which the muscular walls are thinned with little change in the volume of the heart. In some embodiments, the heart failure is associated with a decrease in cardiac muscle mass. In some embodiments, the heart failure is associated with a 5%, 10%, 20%, 30%, 40%, 50%, 60%, 70%, 75%, 80%, or more decrease in cardiac muscle mass.

Muscle atrophy may be caused by any condition. In some embodiments, complexes are effective in treating muscle atrophy caused by cancer cachexia, cardiac cachexia, acute lung injury, denervation, glucocorticoid use, inactivity (muscle disuse), abnormal levels of myostatin, and/or fasting. In some embodiments, muscle atrophy is caused by cancer cachexia, fasting, renal failure, diabetes, denervation, or glucocorticoid-induced muscle atrophy. As a non-limiting example, ultrasounds and/or magnetic resonance imaging (MRI) may be used to diagnose a subject with muscle atrophy. In some embodiments, muscle atrophy affects slow type 1 or fast type 2 muscle fibers in a subject. See, e.g., Talbot et al., Wiley Interdiscip Rev Dev Biol. 2016 July; 5(4):518-34, which is herein incorporated by reference in its entirety. In some embodiments, the subject does not have peripheral artery disease. In some embodiments, the peripheral arterial system of the subject is intact. In some instances, the muscle atrophy may not be localized to the subject's lower or hind limb extremities.

Myotonic dystrophy is a long-term genetic disorder that affects muscle function. Symptoms include gradually worsening muscle loss and weakness. There are two main types of myotonic dystrophy: type 1 (DM1), due to mutations in the DMPK gene, and type 2 (DM2), due to mutations in the CNBP gene.

Parkinson's disease (PD) is a long-term degenerative disorder of the central nervous system that mainly affects the motor system. As the disease worsens, non-motor symptoms become more common. Early in the disease, the most obvious symptoms are shaking, rigidity, slowness of movement, and difficulty with walking. Thinking and behavioral problems may also occur. Dementia becomes common in the advanced stages of the disease. Depression and anxiety are also common, occurring in more than a third of people with PD.

Amyotrophic lateral sclerosis (ALS) causes the death of neurons controlling voluntary muscles. ALS is characterized by stiff muscles, muscle twitching, and gradually worsening weakness due to muscles decreasing in size. It may begin with weakness in the arms or legs, or with difficulty speaking or swallowing. About half of the people affected develop at least mild difficulties with thinking and behavior and most people experience pain. Most eventually lose the ability to walk, use their hands, speak, swallow, and breathe.

In some embodiments, complexes are effective in specifically targeting expression of MEF2D, KLF15, MED1, MED13, or PPP1R3A in cardiac cells. In some embodiments, complexes are effective in treating and/or preventing impaired muscle and cardiac development (e.g. congenital heart defect), a muscular disease (e.g., muscle atrophy, myotonic dystrophy, cachexia), a heart disease (e.g., cardiac atrophy, cardiac hypertrophy, cardiomyopathy, cardiovascular disease, congenital heart disease, heart arrhythmias), heart failure, or a neuromuscular disease (e.g., Parkinson's disease, amyotrophic lateral sclerosis (ALS)).

In some embodiments, the subject that has or is suspected of having impaired muscle and cardiac development has a decreased level of MEF2D, expression and/or activity (e.g., decreased by at least 20%, at least 30%, at least 40%, at least 50%, at least 60%, at least 70%, at least 80%, at least 90%, or more), compared to the MEF2D expression and/or activity level in a healthy subject. In some embodiments, a complex comprising a muscle-targeting agent covalently linked to a molecular payload as described herein is effective in increasing expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in increasing the MEF2D expression and/or activity to the level of a healthy subject.

In some embodiments, the subject that has or is suspected of having a heart disease (e.g., cardiac hypertrophy, cardiomyopathy) has an increased level of MEF2D expression and/or activity (e.g., increased by at least 20%, at least 30%, at least 40%, at least 50%, at least 60%, at least 70%, at least 80%, at least 90%, or more), compared to the MEF2D expression and/or activity level in a healthy subject. In some embodiments, a complex comprising a muscle-targeting agent covalently linked to a molecular payload as described herein is effective in reducing the MEF2D expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in reducing the MEF2D expression and/or activity to the level of a healthy subject. In some embodiments, complexes are effective in specifically targeting expression of MEF2D in cardiac cells.

In some embodiments, the subject that has or is suspected of having Parkinson's disease has a decreased level of MEF2D expression and/or activity (e.g., decreased by at least 20%, at least 30%, at least 40%, at least 50%, at least 60%, at least 70%, at least 80%, at least 90%, or more), compared to the MEF2D expression and/or activity level in a healthy subject. In some embodiments, a complex comprising a muscle-targeting agent covalently linked to a molecular payload as described herein is effective in increasing the MEF2D expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in increasing the MEF2D expression and/or activity to the level of a healthy subject.

In some embodiments, the subject that has or is suspected of having ALS has an increased level of MEF2D expression and/or activity (e.g., increased by at least 20%, at least 30%, at least 40%, at least 50%, at least 60%, at least 70%, at least 80%, at least 90%, or more), compared to the MEF2D expression and/or activity level in a healthy subject. In some embodiments, a complex comprising a muscle-targeting agent covalently linked to a molecular payload as described herein is effective in reducing the MEF2D expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in reducing the MEF2D expression and/or activity to the level of a healthy subject.

In some embodiments, the subject that has or is suspected of having a muscular disease (e.g., muscle atrophy, myotonic dystrophy) expresses an isoform of MEF2D lacking the β-exon (e.g., as described in Lee et al., The Journal of Biological Chemistry, 285, 33779-33787, 2010, incorporated herein by reference). In some embodiments, a complex comprising a muscle-targeting agent covalently linked to a molecular payload as described herein is effective in reducing the MEF2D isoform expression and/or activity by at least 20%, at least 30%, at least 40%, at least 50%, at least 60%, at least 70%, at least 80%, at least 90%, or more.

In some embodiments, the subject that has or is suspected of having impaired muscle and cardiac development has a decreased level of KLF15, expression and/or activity (e.g., decreased by at least 20%, at least 30%, at least 40%, at least 50%, at least 60%, at least 70%, at least 80%, at least 90%, or more), compared to the KLF15 expression and/or activity level in a healthy subject. In some embodiments, a complex comprising a muscle-targeting agent covalently linked to a molecular payload as described herein is effective in increasing expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in increasing the KLF15 expression and/or activity to the level of a healthy subject.

In some embodiments, the subject that has or is suspected of having a heart disease (e.g., cardiac hypertrophy, cardiomyopathy) has a decreased level of KLF15 expression and/or activity (e.g., decreased by at least 20%, at least 30%, at least 40%, at least 50%, at least 60%, at least 70%, at least 80%, at least 90%, or more), compared to the KLF15 expression and/or activity level in a healthy subject. In some embodiments, a complex comprising a muscle-targeting agent covalently linked to a molecular payload as described herein is effective in increasing the KLF15 expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in increasing the KLF15 expression and/or activity to the level of a healthy subject. In some embodiments, complexes are effective in specifically targeting expression of KLF15 in cardiac cells.

In some embodiments, the subject that has heart arrhythmia has an increased level of KLF15 expression and/or activity (e.g., increased by at least 20%, at least 30%, at least 40%, at least 50%, at least 60%, at least 70%, at least 80%, at least 90%, or more), compared to the KLF15 expression and/or activity level in a healthy subject. In some embodiments, a complex comprising a muscle-targeting agent covalently linked to a molecular payload as described herein is effective in reducing the KLF15 expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in reducing the KLF15 expression and/or activity to the level of a healthy subject.

In some embodiments, the subject that has heart arrhythmia has a decreased level of KLF15 expression and/or activity (e.g., decreased by at least 20%, at least 30%, at least 40%, at least 50%, at least 60%, at least 70%, at least 80%, at least 90%, or more), compared to the KLF15 expression and/or activity level in a healthy subject. In some embodiments, a complex comprising a muscle-targeting agent covalently linked to a molecular payload as described herein is effective in increasing the KLF15 expression and/or activity by at least 20%, at least 30%, at least 40%, at least 50%, at least 60%, at least 70%, at least 80%, at least 90%, or more. In some embodiments, a complex comprising a muscle-targeting agent covalently linked to a molecular payload as described herein is effective in increasing the KLF15 expression and/or activity to the level of a healthy subject.

In some embodiments, complexes are effective in treating cardiovascular disease (e.g., congenital heart disease or congenital heart defect) in subjects having a wild-type MED13. In some embodiments, complexes to treat cardiovascular disease promote or enhance the expression or activity of wild-type MED13.

In some embodiments, complexes are effective in treating cardiovascular disease (e.g., congenital heart disease or congenital heart defect) in subjects having a mutated MED13 gene. In some embodiments, complexes to treat cardiovascular disease having mutated MED13 inhibit the expression or activity of mutant MED13. In some embodiments, subjects have a mutated allele of MED13 comprising a missense mutation. In some embodiments, a mutated allele of MED13 encodes a T326I, P327S and/or P327Q mutation. In some embodiments, a mutated allele of MED13 comprises an in-frame deletion (e.g., of nucleotides encoding T326).

In some embodiments, a pharmaceutical composition may comprise more than one complex comprising a muscle-targeting agent covalently linked to a molecular payload. In some embodiments, a pharmaceutical composition may further comprise any other suitable therapeutic agent for treatment of a subject, e.g. a human subject having impaired muscle and cardiac development (e.g. congenital heart defect), a muscular disease (e.g., muscle atrophy, myotonic dystrophy, cachexia), a heart disease (e.g., cardiac atrophy, cardiac hypertrophy, cardiomyopathy, cardiovascular disease, congenital heart disease, heart arrhythmias), heart failure, or a neuromuscular disease (e.g., Parkinson's disease, amyotrophic lateral sclerosis (ALS)). In some embodiments, the other therapeutic agents may enhance or supplement the effectiveness of the complexes described herein. In some embodiments, the other therapeutic agents may function to treat a different symptom or disease than the complexes described herein.

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g., safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment

The efficacy of treatment may be assessed using any suitable methods. In some embodiments, the efficacy of treatment may be assessed by evaluation of observation of symptoms associated with a disease treated with complexes described herein, e.g., irritable bowel syndrome, inflammatory bowel disease, heart failure, muscle atrophy, muscular dystrophies, cachexia, cardiac fibrosis, and/or muscle hypertrophy (including but not limited to cardiac hypertrophy). For example, a subject with muscle atrophy may have difficulty balancing, have been inactive for an extended period, have malnutrition, have a limb that is noticeably smaller in size than another limb, and/or is experiencing weakness. Suitable methods of assessing muscle atrophy include ultrasounds, magnetic resonance imaging, and muscle biopsies. For example, the extent of muscle atrophy may be determined by determining the shape of muscle fibers. Expression and/or activity of a target gene described herein (e.g., MSTN, INHBA, ACVR1B, MLCK1, ACVR1, FBXO32, TRIM63, MEF2D, KLF15, MED1, MED13, or PPP1R3A) may also be assessed using any suitable method. For example, levels of a target mRNA and/or protein may be assayed to determine whether a treatment reduces target gene expression.

In some embodiments, a pharmaceutical composition that comprises a complex comprising a muscle-targeting agent covalently linked to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a single dose or administration of a pharmaceutical composition that comprises a complex comprising a muscle-targeting agent covalently linked to a molecular payload described herein to a subject is sufficient to inhibit activity or expression of a target gene for at least 1-5, 1-10, 5-15, 10-20, 15-30, 20-40, 25-50, or more days. In some embodiments, a single dose or administration of a pharmaceutical composition that comprises a complex comprising a muscle-targeting agent covalently linked to a molecular payload described herein to a subject is sufficient to inhibit activity or expression of a target gene for at least 1, 2, 3, 4, 5, 6, 7, 8, 9, 10, 11, or 12 weeks. In some embodiments, a single dose or administration of a pharmaceutical composition that comprises a complex comprising a muscle-targeting agent covalently linked to a molecular payload described herein to a subject is sufficient to inhibit activity or expression of a target gene for at least 1, 2, 3, 4, 5, or 6 months.

In some embodiments, a pharmaceutical composition may further comprise any other suitable therapeutic agent for treatment of a subject, e.g., a human subject having muscle atrophy or cardiac hypertrophy. In some embodiments, the other therapeutic agents may enhance or supplement the effectiveness of the complexes described herein. In some embodiments, the other therapeutic agents may function to treat a different symptom or disease than the complexes described herein.

### EXAMPLES

### Example 1: Targeting HPRT with Transfected Antisense Oligonucleotides

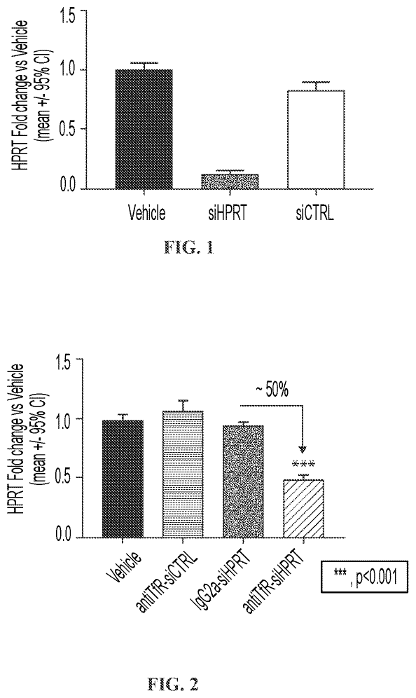

A siRNA that targets hypoxanthine phosphoribosyltransferase (HPRT) was tested in vitro for its ability to reduce expression levels of HPRT in an immortalized cell line. Briefly, Hepa 1-6 cells were transfected with either a control siRNA (siCTRL; 100 nM) or the siRNA that targets HPRT (siHPRT; 100 nM), formulated with Lipofectamine 2000. HPRT expression levels were evaluated 48 hours following transfection. A control experiment was also performed in which vehicle (phosphate-buffered saline) was delivered to Hepa 1-6 cells in culture and the cells were maintained for 48 hours. As shown in FIG. 1, it was found that the HPRT siRNA reduced HPRT expression levels by about 90% compared with controls. Sequences of the siRNAs used are provided in Table 41.

TABLE 41
Sequences of siHPRT and siCTRL

| | | SEQ ID |
|---|---|---|
| | Sequence | NO |
| siHPRT sense | 5′-UcCuAuGaCuGuAg | 371 |
| strand | AuUuUaU-(CH₂)₆NH₂-3′ | |
| siHPRT antisense | 5′-aUaAaAuCuAcAgUc | 372 |
| strand | AuAgGasAsu-3′ | |
| siCTRL sense | 5′-UgUaAuAaCcAuAuC | 373 |
| strand | uAcCuU-(CH₂)₆NH₂-3′ | |
| siCTRL antisense | 5′-aAgGuAgAuAuGgUu | 374 |
| strand | AuUaCasAsa-3′ | |

*Lower case-2′-O-Me ribonucleoside; Capital letter-2′-F ribonucleoside; s-phosphorothioate linkage

### Example 2: Targeting HPRT with a Muscle-Targeting Complex

A muscle-targeting complex was generated comprising the HPRT siRNA used in Example 1 (siHPRT) covalently linked, via a non-cleavable N-gamma-maleimidobutyryl-oxysuccinimide ester (GMBS) linker, to RI7 217 anti-TfR1 Fab (DTX-A-002), an anti-transferrin receptor antibody.

Briefly, the GMBS linker was dissolved in dry DMSO and coupled to the 3′ end of the sense strand of siHPRT through amide bond formation under aqueous conditions. Completion of the reaction was verified by Kaiser test. Excess linker and organic solvents were removed by gel permeation chromatography. The purified, maleimide functionalized sense strand of siHPRT was then coupled to DTX-A-002 antibody using a Michael addition reaction.

The product of the antibody coupling reaction was then subjected to size exclusion chromatography (SEC) purification. antiTfR-siHPRT complexes comprising one or two siHPRT molecules covalently linked to DTX-A-002 antibody were purified. Densitometry confirmed that the purified sample of complexes had an average siHPRT to antibody ratio of 1.46. SDS-PAGE analysis demonstrated that >90% of the purified sample of complexes comprised DTX-A-002 linked to either one or two siHPRT molecules.

Using the same methods as described above, a control IgG2a-siHPRT complex was generated comprising the HPRT siRNA used in Example 1 (siHPRT) covalently linked via the GMBS linker to an IgG2a (Fab) antibody (DTX-A-003). Densitometry confirmed that DTX-C-001 (the control IgG2a-siHPRT complex) had an average siHPRT to antibody ratio of 1.46 and SDS-PAGE demonstrated that >90% of the purified sample of control complexes comprised DTX-A-003 linked to either one or two siHPRT molecules.

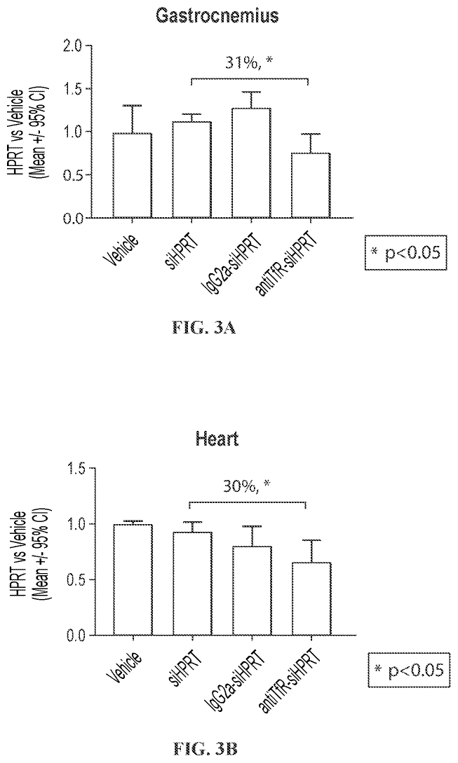

The antiTfR-siHPRT complex was then tested for cellular internalization and inhibition of HPRT in cellulo. Hepa 1-6 cells, which have relatively high expression levels of transferrin receptor, were incubated in the presence of vehicle (phosphate-buffered saline), IgG2a-siHPRT (100 nM), antiTfR-siCTRL (100 nM), or antiTfR-siHPRT (100 nM), for 72 hours. After the 72 hour incubation, the cells were isolated and assayed for expression levels of HPRT (FIG. 2). Cells treated with the antiTfR-siHPRT demonstrated a reduction in HPRT expression by about 50% relative to the cells treated with the vehicle control and to those treated with the IgG2a-siHPRT complex. Meanwhile, cells treated with either of the IgG2a-siHPRT or antiTfR-siCTRL had HPRT expression levels comparable to the vehicle control (no reduction in HPRT expression). These data indicate that the anti-transferrin receptor 1 antibody of the antiTfR-siHPRT enabled cellular internalization of the complex, thereby allowing the siHPRT to inhibit expression of HPRT.

### Example 3: Targeting HPRT in Mouse Muscle Tissues with a Muscle-Targeting Complex

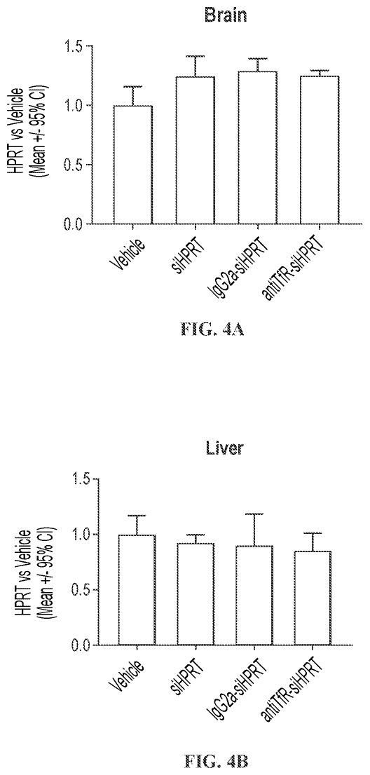

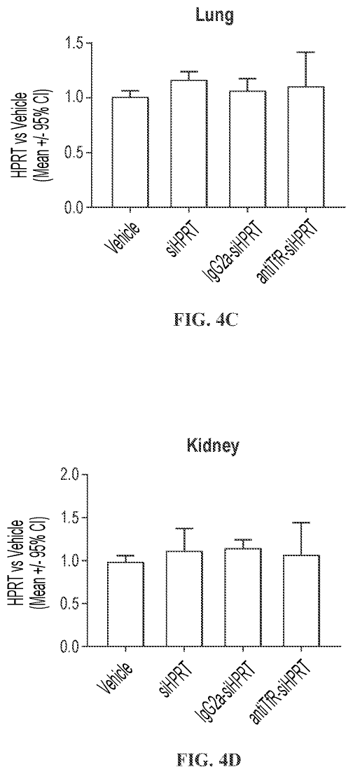

The muscle-targeting complex described in Example 2, antiTfR-siHPRT, was tested for inhibition of HPRT in mouse tissues. C57BL/6 wild-type mice were intravenously injected with a single dose of a vehicle control (phosphate-buffered saline); siHPRT (2 mg/kg of RNA); IgG2a-siHPRT (2 mg/kg of RNA, corresponding to 9 mg/kg antibody complex); or antiTfR-siHPRT (2 mg/kg of RNA, corresponding to 9 mg/kg antibody complex). Each experimental condition was replicated in four individual C57BL/6 wild-type mice. Following a three-day period after injection, the mice were euthanized and segmented into isolated tissue types. Individual tissue samples were subsequently assayed for expression levels of HPRT (FIGS. 3A-3B and 4A-4E).

Mice treated with the antiTfR-siHPRT complex demonstrated a reduction in HPRT expression in heart (30% reduction; p<0.05) and gastrocnemius (31% reduction; p<0.05), relative to the mice treated with the siHPRT control (FIGS. 3A-3B). Meanwhile, mice treated with the IgG2a-siHPRT complex had HPRT expression levels comparable to the siHPRT control (little or no reduction in HPRT expression) for all assayed muscle tissue types.

Mice treated with the antiTfR-siHPRT complex demonstrated no change in HPRT expression in non-muscle tissues such as brain, liver, lung, kidney, and spleen tissues (FIGS. 4A-4E).

These data indicate that the anti-transferrin receptor 1 antibody of the antiTfR-siHPRT complex enabled cellular internalization of the complex into muscle-specific tissues in an in vivo mouse model, thereby allowing the siHPRT to inhibit expression of HPRT. These data further demonstrate that the antiTfR-oligonucleotide complexes of the current disclosure are capable of specifically targeting muscle tissues.

### Example 4: Targeting MSTN with a Muscle-Targeting Complex

A muscle-targeting complex is generated comprising an antisense oligonucleotide that targets an allele of MSTN (MSTN ASO) covalently linked, via a cathepsin cleavable linker, to DTX-A-002 (RI7 217 Fab), an anti-transferrin receptor 1 antibody.

Briefly, a maleimidocaproyl-L-valine-L-citrulline-p-aminobenzyl alcohol p-nitrophenyl carbonate (MC-Val-Cit-PABC-PNP) linker molecule is coupled to NH₂—C₆-DNM2 ASO using an amide coupling reaction. Excess linker and organic solvents are removed by gel permeation chromatography. The purified Val-Cit-linker-DNM2 ASO is then coupled to a thiol on the anti-transferrin receptor 1 antibody (DTX-A-002).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising MSTN ASO covalently linked via a Val-Cit linker to an IgG2a (Fab) antibody. The purified muscle-targeting complex comprising DTX-A-002 covalently linked to MSTN ASO is then tested for cellular internalization and inhibition of MSTN. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of MSTN.

### Example 5: Identification of Candidate Oligonucleotides for Inhibiting MSTN

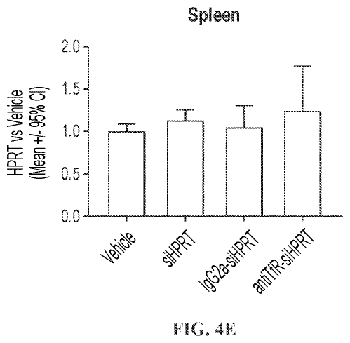

To identify candidate oligonucleotides for inhibiting MSTN, siRNAs were screened for suppression of MSTN expression. Cells were treated with 0.1 nM or 10 nM of each siRNA and gene expression was measured. The siRNAs Each siRNA was designed to have cross-species activity. FIG. 5 shows MSTN gene expression data for each of the 24 siRNAs tested at both doses. Four siRNAs from this screen (hsMSTN-1, hsMSTN-2, hsMSTN-3, and hsMSTN-4) were tested further in a dose response analysis described in Example 6.

### Example 6: Inhibiting MSTN with Candidate Oligonucleotides

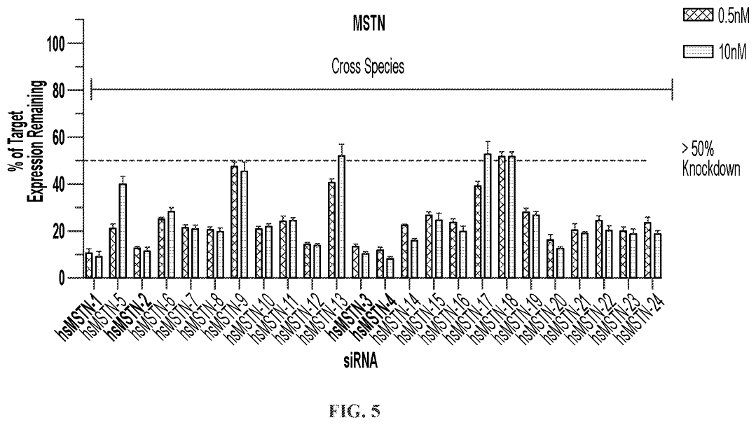

To evaluate candidate oligonucleotides for inhibiting human MSTN, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC50 and IC80 values for each oligonucleotide. FIG. 6 shows dose response curves for inhibition of MSTN and Table 42 shows the corresponding IC50 and IC80 values.

TABLE 42
Oligonucleotide inhibition of human MSTN

| | | | SEQ | Antisense | SEQ | | |
|---|---|---|---|---|---|---|---|
| | | Sense Sequence | ID | Sequence | ID | IC50 | IC80 |
| Target | siRNA # | (5′ to 3′) | NO: | (5′ to 3′) | NO: | (pM) | (pM) |
| Human | hsMSTN-1* | mUmCfUmUfUmGfGm | 173 | fAfUmAfAmUfCmGfUm | 197 | 84 | 597 |
| MSTN | | AfAmGfAmUfGmAfCm | | CfAmUfCmUfUmCfCm | | | |
| | | GfAmUfUmAfU | | AfAmAfGmA*fG*mC | | | |
| | hsMSTN-2* | mGmAfAmGfAmUfGm | 175 | fAfGmCfGmUfGmAfUm | 199 | 120 | 902 |
| | | AfCmGfAmUfUmAfUm | | AfAmUfCmGfUmCfAm | | | |
| | | CfAmCfGmCfU | | UfCmUfUmC*fC*mA | | | |
| | hsMSTN-3* | mGmAfCmAfGmUfGm | 184 | fAfGmCfCmAfAmUfUm | 208 | 101 | 979 |
| | | UfUmGfCmAfAmAfAm | | UfUmGfCmAfAmCfAm | | | |
| | | UfUmGfGmCfU | | CfUmGfUmC*fU*mU | | | |
| | hsMSTN-4* | mGmUfGmUfUmGfCm | 185 | fUfUmUfGmAfGmCfCm | 209 | 122 | 948 |
| | | AfAmAfAmUfUmGfGm | | AfAmUfUmUfUmGfCm | | | |
| | | CfUmCfAmAfA | | AfAmCfAmC*fU*mG | | | |

* Oligonucleotide designed to have cross-species activity
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate
internucleoside linkage, and lack of “*” between nucleosides indicate phosphodiester internucleoside linkage.

### Example 7: Screening INHBA Candidate siRNAs

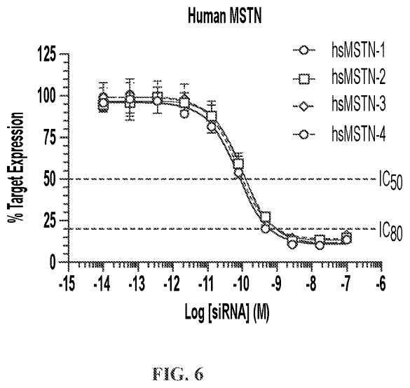

To identify candidate oligonucleotides for inhibiting INHBA, siRNAs were screened for suppression of INHBA expression. Cells were treated with 0.1 nM or 10 nM of each siRNA and gene expression was measured. Each siRNA was designed to have cross-species activity. FIG. 7 shows INHBA gene expression data for each of the 24 siRNAs tested at both doses. Three siRNAs (hsINHBA-1, hsINHBA-2, and hsINHBA-3) from this screen were tested further in a dose response analysis described in Example 8.

### Example 8: Inhibiting INHBA with Candidate Oligonucleotides

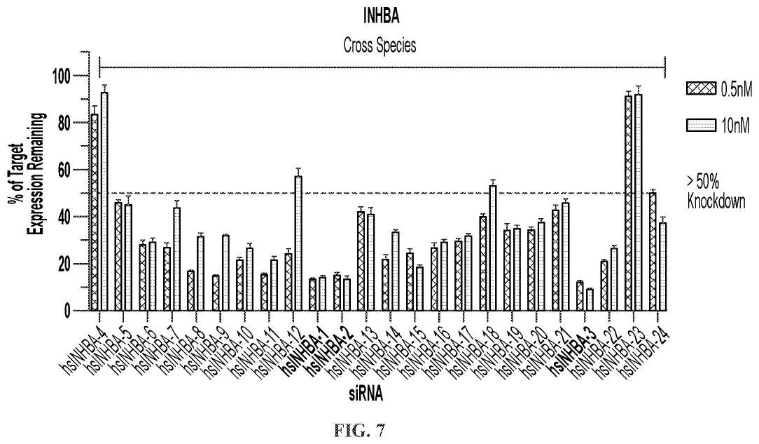

To evaluate candidate oligonucleotides for inhibiting human INHBA, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC50 and IC80 values for each oligonucleotide. FIG. 8 shows dose response curves for inhibition of INHBA and Table 43 shows the corresponding IC50 and IC80 values.

TABLE 43
Oligonucleotide inhibition of human INHBA

| | | | SEQ | | SEQ | | |
|---|---|---|---|---|---|---|---|
| | | Sense Sequence | ID | Antisense Sequence | ID | IC50 | IC80 |
| Target | siRNA # | (5′ to 3′) | NO: | (5′ to 3′) | NO: | (pM) | (pM) |
| Human | hsINHBA-1* | mGmGfCmAfAmGfUm | 304 | fAfCmUfAmUfAmAfUmCfC | 328 | 315 | 5674 |
| INHBA | | UfGmCfUmGfGmAfU | | mAfGmCfAmAfCmUfUmGf | | | |
| | | mUfAmUfAmGfU | | CmC*fA*mA | | | |
| | hsINHBA-2* | mAmAfGmUfUmGfCm | 305 | fCfUmCfAmCfUmAfUmAfA | 329 | 117 | 4801 |
| | | UfGmGfAmUfUmAfU | | mUfCmCfAmGfCmAfAmCf | | | |
| | | mAfGmUfGmAfG | | UmU*fG*mC | | | |
| | hsINHBA-3* | mCmUfGmCfUmGfUm | 315 | fAfAmGfAmAfCmUfGmUfU | 339 | 238 | 12679 |
| | | AfAmGfAmAfAmCfA | | mUfCmUfUmAfCmAfGmCf | | | |
| | | mGfUmUfCmUfU | | AmG*fA*mU | | | |

* Oligonucleotide designed to have cross-species activity
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

### Example 9: Screening ACVR1B Candidate siRNAs

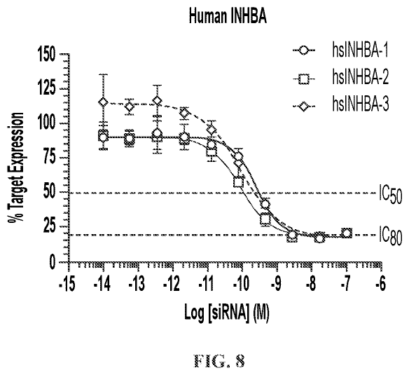

To identify candidate oligonucleotides for inhibiting ACVR1B, siRNAs were screened for suppression of ACVR1B expression. Cells were treated with 0.1 nM or 10 nM of each siRNA and gene expression was measured. Each siRNA was designed to have cross-species activity, activity against human and cynomolgus sequences, or activity against rat and mouse sequences. FIG. 9 shows ACVR1B gene expression data for each of the 24 siRNAs tested at both doses. Five siRNAs from this screen (hsACVR1B-1 and hsACVR1B-2; and mmACVR1B-1, mmACVR1B-2 and mmACVR1B-3) were tested further in a dose response analysis described in Example 10.

### Example 10: Inhibiting ACVR1B with Candidate Oligonucleotides

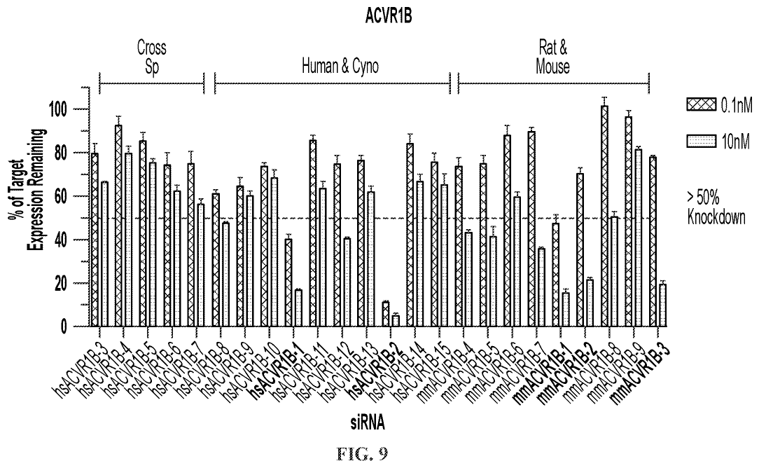

To evaluate candidate oligonucleotides for inhibiting human and murine ACVR1B, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC50 and IC80 values for each oligonucleotide. FIGS. 10 (human) and 11 (murine) show dose response curves for inhibition of ACVR1B and Tables 44 (human) and 45 (murine) show the corresponding IC50 and IC80 values.

TABLE 44
Oligonucleotide inhibition of human ACVR1B

| | | | SEQ | Antisense | SEQ | | |
|---|---|---|---|---|---|---|---|
| | | Sense Sequence | ID | Sequence | ID | IC50 | IC80 |
| Target | siRNA # | (5′ to 3′) | NO: | (5′ to 3′) | NO: | (pM) | (pM) |
| Human | hsACVR1B-1 | mCmUfCmCfAmGfG | 253 | fAfUmCfGmUfAmGfAm | 351 | 272 | # |
| ACVR1B | | mAfUmCfUmUfGmU | | CfAmAfGmAfUmCfCm | | | |
| | | fCmUfAmCfGmAfU | | UfGmGfAmG*fC*mG | | | |
| | hsACVR1B-2 | mCmAfUmCfAmUfU | 257 | fAfUmGfAmCfAmAfGm | 355 | 53 | 273 |
| | | mGfUmUfUmUfCmC | | GfAmAfAmAfCmAfAm | | | |
| | | fUmUfGmUfCmAfU | | UfGmAfUmG*fA*mU | | | |

# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 45
Oligonucleotide inhibition of murine ACVR1B

| | | | SEQ | Antisense | SEQ | | |
|---|---|---|---|---|---|---|---|
| | | Sense Sequence | ID | Sequence | ID | IC₅₀ | IC₈₀ |
| Target | siRNA # | (5′ to 3′) | NO: | (5′ to 3′) | NO: | (pM) | (pM) |
| Murine | mmACVR1B-1 | mCmAfCmAfCmUfG | 264 | fAfGmUfCmAfAmUfAm | 362 | 247 | # |
| ACVR1B | | mCfUmGfCmUfAmUf | | UfAmGfCmAfGmCfAmG | | | |
| | | AmUfUmGfAmCfU | | fUmGfUmG*fU*mG | | | |
| | mmACVR1B-2 | mGmGfUmCfUmCfC | 265 | fUfCmCfAmGfGmUfUm | 363 | 754 | # |
| | | mAfUmCfUmUfUmAf | | AfAmAfGmAfUmGfGm | | | |
| | | AmCfCmUfGmGfA | | AfGmAfCmC*fA*mU | | | |
| | mmACVR1B-3 | mAmCfAmAfGmAfG | 268 | fCfCmCfUmUfGmCfCm | 366 | 1140 | # |
| | | mAfUmUfAmUfCmGf | | GfAmUfAmAfUmCfUmC | | | |
| | | GmCfAmAfGmGfG | | fUmUfGmU*fA*mA | | | |

# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

### Example 11: Targeting MLCK1 with a Muscle-Targeting Complex

A muscle-targeting complex is generated comprising an antisense oligonucleotide that targets a mutant allele of MLCK1 covalently linked, via a cathepsin cleavable linker, to DTX-A-002 (RI7 217 Fab), an anti-transferrin receptor 1 antibody.

Briefly, a maleimidocaproyl-L-valine-L-citrulline-p-aminobenzyl alcohol p-nitrophenyl carbonate (MC-Val-Cit-PABC-PNP) linker molecule is coupled to NH₂—C₆-MLCK1 ASO using an amide coupling reaction. Excess linker and organic solvents are removed by gel permeation chromatography. The purified Val-Cit-linker-MLCK1 ASO is then coupled to a thiol on the anti-transferrin receptor 1 antibody (DTX-A-002).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising MLCK1 ASO covalently linked via a Val-Cit linker to an IgG2a (Fab) antibody.

The purified muscle-targeting complex comprising DTX-A-002 covalently linked to MLCK1 ASO is then tested for cellular internalization and inhibition of MLCK1. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of MLCK1.

### Example 12: Targeting ACVR1 with a Muscle-Targeting Complex

A muscle-targeting complex is generated comprising an antisense oligonucleotide that targets a mutant allele of ACVR1 (ACVR1 ASO) covalently linked, via a cathepsin cleavable linker, to DTX-A-002 (RI7 217 Fab), an anti-transferrin receptor 1 antibody.

Briefly, a maleimidocaproyl-L-valine-L-citrulline-p-aminobenzyl alcohol p-nitrophenyl carbonate (MC-Val-Cit-PABC-PNP) linker molecule is coupled to NH₂—C₆-ACVR1 ASO using an amide coupling reaction. Excess linker and organic solvents are removed by gel permeation chromatography. The purified Val-Cit-linker-ACVR1 ASO is then coupled to a thiol on the anti-transferrin receptor 1 antibody (DTX-A-002).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising ACVR1 ASO covalently linked via a Val-Cit linker to an IgG2a (Fab) antibody.

The purified muscle-targeting complex comprising DTX-A-002 covalently linked to ACVR1 ASO is then tested for cellular internalization and inhibition of ACVR1. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression analysis of ACVR1.

### Example 13: Knockdown of ACVR1 Expression with Candidate Oligonucleotides

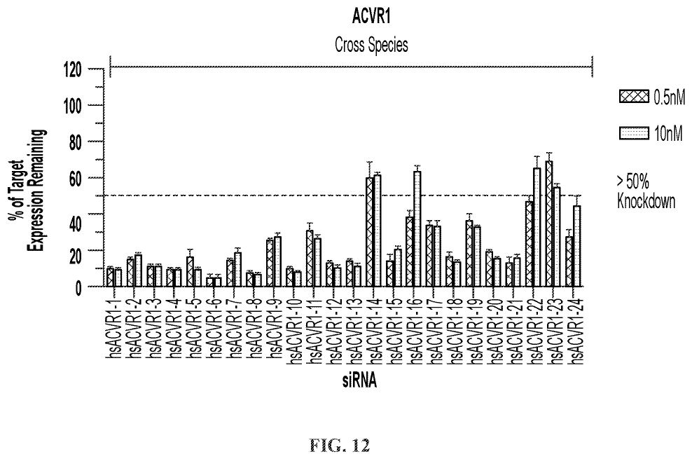

To identify lead oligonucleotide candidates, a panel of oligonucleotide complexes generated to target ACVR1 were screened for knockdown of ACVR1 expression in cell culture. Expression knockdown was assayed at 10 nM and 0.5 nM. All assayed ACVR siRNA oligonucleotides were generated with cross-species specificity (i.e., expected to target both human/cynomolgus and mouse/rat ACVR1 based on sequence similarity) and tested against human ACVR1. Candidate oligonucleotides exhibiting the greatest reduction in target expression were subsequently selected for further dose response analysis. FIG. 12 shows ACVR1 expression knockdown for screened oligonucleotides.

### Example 14: Inhibiting ACVR1 with Candidate Oligonucleotides

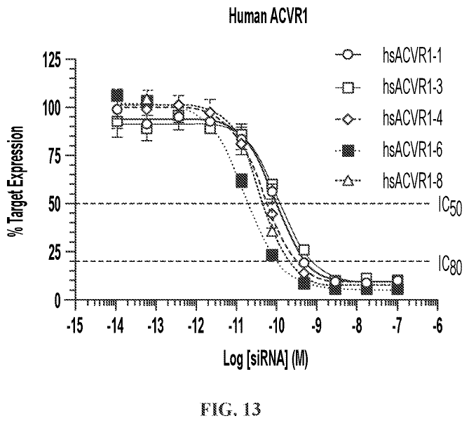

To evaluate candidate oligonucleotides for inhibiting human ACVR1, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC₅₀ and IC₈₀ values for each oligonucleotide. FIG. 13 shows dose response curves for inhibition of ACVR1 and Table 46 shows the corresponding IC₅₀ and IC₈₀ values.

TABLE 46
Oligonucleotide inhibition of human ACVR1

| | | | SEQ | Antisense | SEQ | | |
|---|---|---|---|---|---|---|---|
| | | | ID | Sequence | ID | IC₅₀ | IC₈₀ |
| Target | siRNA # | Sense Sequence | NO: | | NO: | (pM) | (pM) |
| Human | hsACVR1-1* | mAmGfCmAfAmAf | 473 | fAfAmUfUmCfUmAf | 497 | 95 | 484 |
| ACVR1 | | GmAfUmUfUmCfA | | CmUfGmAfAmAfU | | | |
| | | mGfUmAfGmAfAm | | mCfUmUfUmGfCm | | | |
| | | UfU | | U*fU*mC | | | |
| | hsACVR1-3* | mAmAfGmCfAmAf | 472 | fAfUmUfCmUfAmCf | 496 | 123 | 694 |
| | | AmGfAmUfUmUfC | | UmGfAmAfAmUfC | | | |
| | | mAfGmUfAmGfAm | | mUfUmUfGmCfUm | | | |
| | | AfU | | U*fC*mU | | | |
| | hsACVR1-4* | mGmAfAmGfCmAf | 471 | fUfUmCfUmAfCmUf | 495 | 57 | 295 |
| | | AmAfGmAfUmUfU | | GmAfAmAfUmCfU | | | |
| | | mCfAmGfUmAfGm | | mUfUmGfCmUfUm | | | |
| | | AfA | | C*fU*mA | | | |
| | hsACVR1-6* | mCmUfUmUfGmCf | 465 | fAfGmUfAmAfUmA | 489 | 19 | 99 |
| | | AmAfAmUfUmAfU | | fAmAfUmAfAmUfU | | | |
| | | mUfUmAfUmUfAm | | mUfGmCfAmAfAm | | | |
| | | CfU | | G*fU*mU | | | |
| | hsACVR1-8* | mCmAfUmUfUmUf | 466 | fAfAmCfUmGfAmUf | 490 | 46 | 178 |
| | | CmUfUmUfUmAfU | | AmAfUmAfAmAfA | | | |
| | | mUfAmUfCmAfGm | | mGfAmAfAmAfUm | | | |
| | | UfU | | G*fU*mC | | | |

# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

### Example 15: Targeting FBXO32 with a Muscle-Targeting Complex

A muscle-targeting complex is generated comprising an antisense oligonucleotide that targets an allele of FBXO32 (FBXO32 ASO) covalently linked, via a cathepsin cleavable linker, to DTX-A-002 (RI7 217 (Fab)), an anti-transferrin receptor 1 antibody.

Briefly, a maleimidocaproyl-L-valine-L-citrulline-p-aminobenzyl alcohol p-nitrophenyl carbonate (MC-Val-Cit-PABC-PNP) linker molecule is coupled to NH₂—C₆—FBXO32 ASO using an amide coupling reaction. Excess linker and organic solvents are removed by gel permeation chromatography. The purified Val-Cit-linker-FBXO32 ASO is then coupled to a thiol on the anti-transferrin receptor 1 antibody (DTX-A-002).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising FBXO32 ASO covalently linked via a Val-Cit linker to an IgG2a (Fab) antibody. The purified muscle-targeting complex comprising DTX-A-002 covalently linked to FBXO32 ASO is then tested for cellular internalization and inhibition of FBXO32. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of FBXO32.

### Example 16: Targeting TRIM63 with a Muscle-Targeting Complex

A muscle-targeting complex is generated comprising an antisense oligonucleotide that targets an allele of TRIM63 (TRIM63 ASO) covalently linked, via a cathepsin cleavable linker, to DTX-A-002 (RI7 217 (Fab)), an anti-transferrin receptor 1 antibody.

Briefly, a maleimidocaproyl-L-valine-L-citrulline-p-aminobenzyl alcohol p-nitrophenyl carbonate (MC-Val-Cit-PABC-PNP) linker molecule is coupled to NH₂—C₆-TRIM63 ASO using an amide coupling reaction. Excess linker and organic solvents are removed by gel permeation chromatography. The purified Val-Cit-linker-TRIM63 ASO is then coupled to a thiol on the anti-transferrin receptor 1 antibody (DTX-A-002).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising TRIM63 ASO covalently linked via a Val-Cit linker to an IgG2a (Fab) antibody.

The purified muscle-targeting complex comprising DTX-A-002 covalently linked to TRIM63 ASO is then tested for cellular internalization and inhibition of TRIM63. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of TRIM63.

### Example 17. Identification of Candidate Oligonucleotides for Inhibiting Human and Murine FBXO32

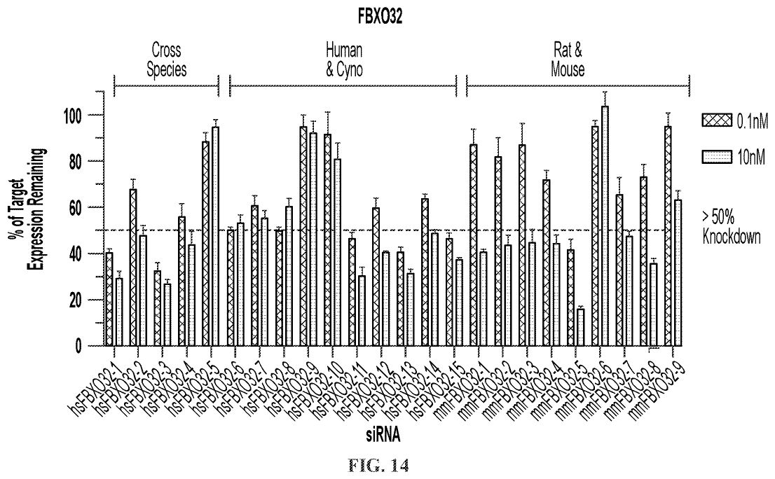

To identify candidate oligonucleotides for inhibiting human, cynomolgus primate, and murine FBXO32, siRNA molecules were screened in a dual luciferase reporter assay. Candidate siRNA molecules were evaluated at a concentration of 0.1 nM and a concentration of 10 nM. Gene inhibition results were used to identify candidate siRNA molecules for further evaluation. FIG. 14 shows percent of target gene expression remaining following treatment with candidate siRNA molecules at a concentration of either 0.1 nM or 10 nM.

### Example 18. Identification of Candidate Oligonucleotides for Inhibiting Human and Murine TRIM63

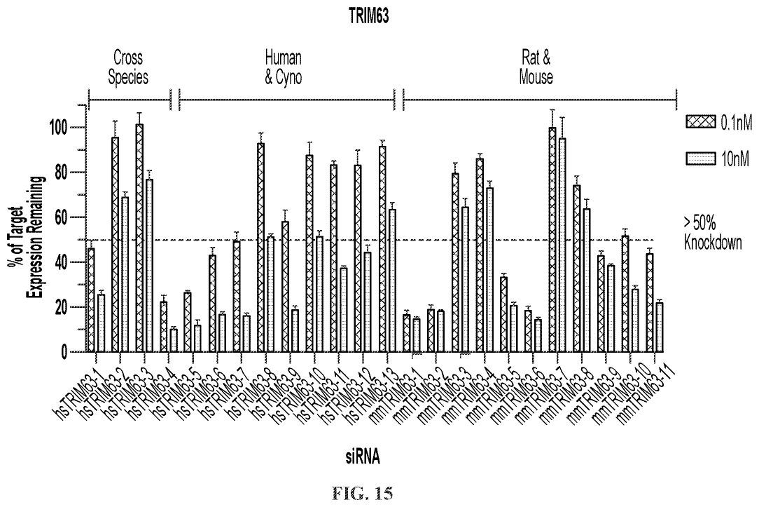

To identify candidate oligonucleotides for inhibiting human, cynomolgus primate, and murine TRIM63, siRNA molecules were screened in a dual luciferase reporter assay. Candidate siRNA molecules were evaluated at a concentration of 0.1 nM and a concentration of 10 nM. Gene inhibition results were used to identify candidate siRNA molecules for further evaluation. FIG. 15 shows percent of target gene expression remaining following treatment with candidate siRNA molecules at a concentration of either 0.1 nM or 10 nM.

### Example 19. Oligonucleotide Inhibition of FBXO32

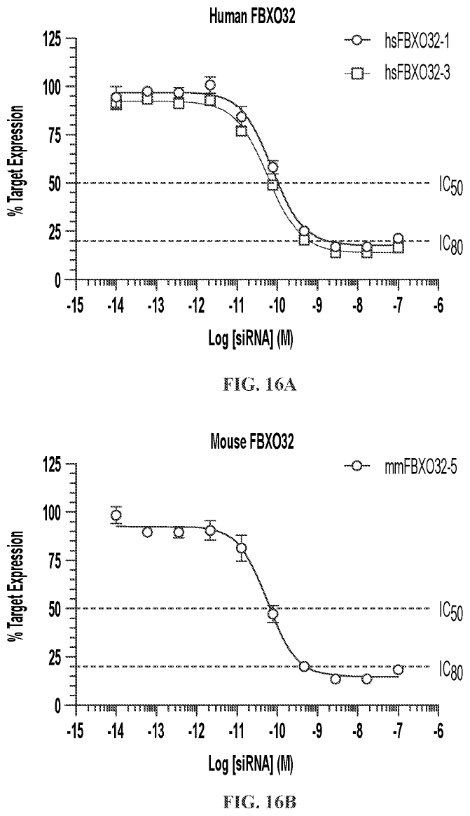

To evaluate candidate oligonucleotides for inhibiting human and murine FBXO32, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC₅₀ and IC₈₀ values for each oligonucleotide. FIGS. 16A (human) and 16B (murine) show dose response curves for inhibition of FBXO32 and Tables 47 (human) and 48 (murine) show the corresponding IC₅₀ and IC₈₀ values.

TABLE 47
Oligonucleotide inhibition of human FBXO32

| | | Sense Sequence | Antisense Sequence | IC₅₀ | IC₈₀ |
|---|---|---|---|---|---|
| Target | siRNA # | (5′to 3′) | (5′to 3′) | (pM) | (pM) |
| Human | hsFBXO32- | mAmGfCmUfGmGfAmUf | fAfCmAfUmCfUmUfCm | 105 | 1792 |
| FBXO32 | 1 | UmGfGmAfAmGfAmAfG | UfUmCfCmAfAmUfCmC | | |
| | | mAfUmGfU (SEQ ID NO: | fAmGfCmU*fG*mC | | |
| | | 531) | (SEQ ID NO: 555) | | |
| | hsFBXO32- | mGmCfUmGfGmAfUmUf | fUfAmCfAmUfCmUfUm | 68 | 622 |
| | 3 | GmGfAmAfGmAfAmGfA | CfUmUfCmCfAmAfUmC | | |
| | | mUfGmUfA (SEQ ID NO: | fCmAfGmC*fU*mG | | |
| | | 533) | (SEQ ID NO: 557) | | |

+ Oligonucleotide designed to have cross-species activity, ‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 48
Oligonucleotide inhibition of murine FBXO32

| | | Sense Sequence | Antisense Sequence | IC₅₀ | IC₈₀ |
|---|---|---|---|---|---|
| Target | siRNA # | (5′to 3′) | (5′to 3′) | (pM) | (pM) |
| Murine | mmFBXO32- | mUmUfCmCfAmGf | fAfCmGfUmAfGmUfU | 69 | 538 |
| FBXO32 | 5 | AmAfGmAfUmUfC | mGfAmAfUmCfUmUfC | | |
| | | mAfAmCfUmAfCm | mUfGmGfAmA*fU*mC | | |
| | | GfU (SEQ ID NO: | (SEQ ID NO: 574) | | |
| | | 550) | | | |

+ Oligonucleotide designed to have cross-species activity, wherein ‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

### Example 20. Oligonucleotide Inhibition of TRIM63

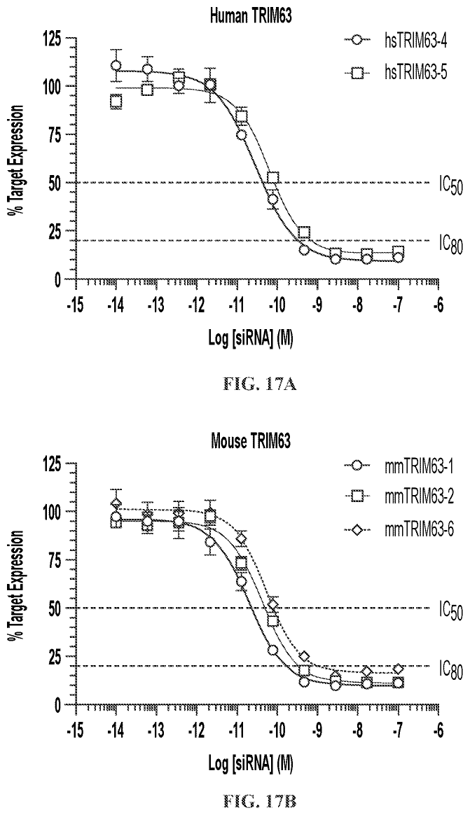

To evaluate candidate oligonucleotides for inhibiting human and murine TRIM63, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC₅₀ and IC₈₀ values for each oligonucleotide. FIGS. 17A (human) and 17B (murine) show dose response curves for inhibition of TRIM63 and Tables 49 (human) and 50 (murine) show the corresponding IC₅₀ and IC₈₀ values.

TABLE 49
Oligonucleotide inhibition of human TRIM63

| | | Sense Sequence | Antisense Sequence | IC₅₀ | IC₈₀ |
|---|---|---|---|---|---|
| Target | siRNA # | (5′to 3′) | (5′to 3′) | (pM) | (pM) |
| Human | hsTRIM63- | mUmGfAmGfUmUfUm | fAfCmAfAmCfAmUfAm | 45 | 332 |
| TRIM63 | 4 | GfUmGfUmUfAmUfAm | UfAmAfCmAfCmAfAm | | |
| | | UfGmUfUmGfU (SEQ | AfCmUfCmA*fG*mU | | |
| | | ID NO: 608) | (SEQ ID NO: 632) | | |
| | hsTRIM63- | mGmUfAmAfAmGfGm | fAfUmUfAmUfAmCfAm | 88 | 686 |
| | 5 | AfAmCfUmUfUmUfGm | AfAmAfGmUfUmCfCm | | |
| | | UfAmUfAmAfU (SEQ | UfUmUfAmC*fA*mA | | |
| | | ID NO: 609) | (SEQ ID NO: 633) | | |

+ Oligonucleotide designed to have cross-species activity, wherein ‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

TABLE 50
Oligonucleotide inhibition of murine TRIM63

| | | Sense Sequence | Antisense Sequence | IC₅₀ | IC₈₀ |
|---|---|---|---|---|---|
| Target | siRNA # | (5′to 3′) | (5′to 3′) | (pM) | (pM) |
| Murine | mmTRIM63- | mUmGfAmAfUmUfU | fAfCmAfAmCfAmUfAm | 24 | 169 |
| TRIM63 | 1 | mGfUmGfUmUfAmUf | UfAmAfCmAfCmAfAm | | |
| | | AmUfGmUfUmGfU | AfUmUfCmA*fG*mU | | |
| | | (SEQ ID NO: 618) | (SEQ ID NO: 642) | | |
| | mmTRIM63- | mAmGfGmAfCmUfG | fAfUmAfUmAfAmCfAm | 51 | 367 |
| | 2 | mAfAmUfUmUfGmUf | CfAmAfAmUfUmCfAm | | |
| | | GmUfUmAfUmAfU | GfUmCfCmU*fA*mU | | |
| | | (SEQ ID NO: 619) | (SEQ ID NO: 643) | | |
| | mmTRIM63- | mAmUfAmGfGmAfC | fAfUmAfAmCfAmCfAm | 84 | 1093 |
| | 6 | mUfGmAfAmUfUmUf | AfAmUfUmCfAmGfUm | | |
| | | GmUfGmUfUmAfU | CfCmUfAmU*fC*mC | | |
| | | (SEQ ID NO: 623) | (SEQ ID NO: 647) | | |

+ Oligonucleotide designed to have cross-species activity, wherein ‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage.

### Example 21: Targeting Genes Associated with Heart Disease with a Muscle-Targeting Complex

Muscle-targeting complexes are generated that each comprise an antisense oligonucleotide (ASO) that targets MEF2D, KLF15, MED1, MED13, or PPP1R3A that is covalently linked, via a cathepsin cleavable linker, to DTX-A-002 (RI7 217 Fab), an anti-transferrin receptor 1 antibody. A muscle-targeting complex that targets KLF15 is generated comprising a guide RNA (gRNA) oligonucleotide. A muscle-targeting complex that targets MED13 targets a mutant allele of MED13. A muscle-targeting complex that targets PPP1R3A is generated comprising an siRNA oligonucleotide.

Briefly, a maleimidocaproyl-L-valine-L-citrulline-p-aminobenzyl alcohol p-nitrophenyl carbonate (MC-Val-Cit-PABC-PNP) linker molecule is coupled to NH₂—C₆-MEF2D ASO, NH₂—C₆-KLF15 ASO, NH₂—C₆-MED1 ASO, NH₂—C₆-MED13 ASO, or NH₂—C₆-PPP1R3A ASO using an amide coupling reaction. Excess linker and organic solvents are removed by gel permeation chromatography. The purified Val-Cit-linker-MEF2D ASO, Val-Cit-linker-KLF15 ASO, Val-Cit-linker-MED1 ASO, Val-Cit-linker-MED13 ASO, or Val-Cit-linker-PPP1R3A ASO is then coupled to a thiol on the anti-transferrin receptor 1 antibody (DTX-A-002).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising the oligonucleotide covalently linked via a Val-Cit linker to an IgG2a (Fab) antibody. The purified muscle-targeting complex comprising DTX-A-002 covalently linked to MEF2D ASO is then tested for cellular internalization and either inhibition of MEF2D, enhancement of KLF15 expression and/or function, enhanced expression of MED1, inhibition of MED13, or inhibition of PPP1R3A expression. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of MEF2D, KLF15, MED1, MED13, or PPP1R3A.

### Example 22: Knockdown of MEF2D Expression with Candidate Oligonucleotides

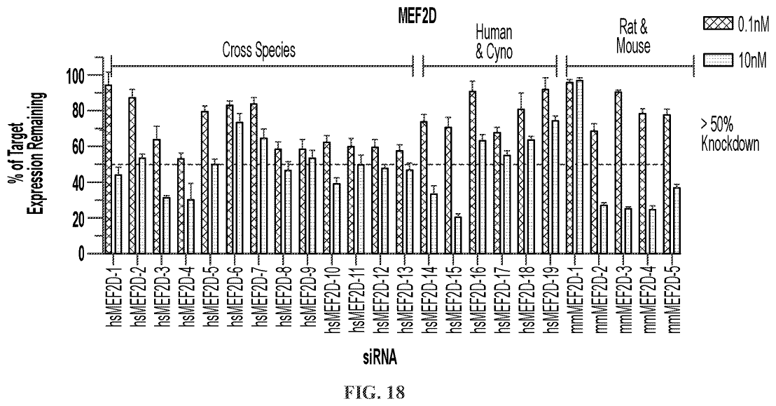

To identify lead oligonucleotide candidates, a panel of oligonucleotide complexes generated to target MEF2D were screened for knockdown of MEF2D expression in cell culture. Expression knockdown was assayed at 10 nM and 0.1 nM. Assayed MEF2D siRNA oligonucleotides were generated with cross-species specificity (i.e., expected to target both human/cynomolgus and mouse/rat MEF2D based on sequence similarity) or specificity for either human/cynomolgus or mouse/rat MEF2D and tested against human or murine MEF2D as appropriate. Candidate oligonucleotides exhibiting the greatest reduction in target expression were subsequently selected for further dose response analysis. FIG. 18 shows MEF2D expression knockdown for screened oligonucleotides.

### Example 23: Knockdown of KLF15 Expression with Candidate Oligonucleotides

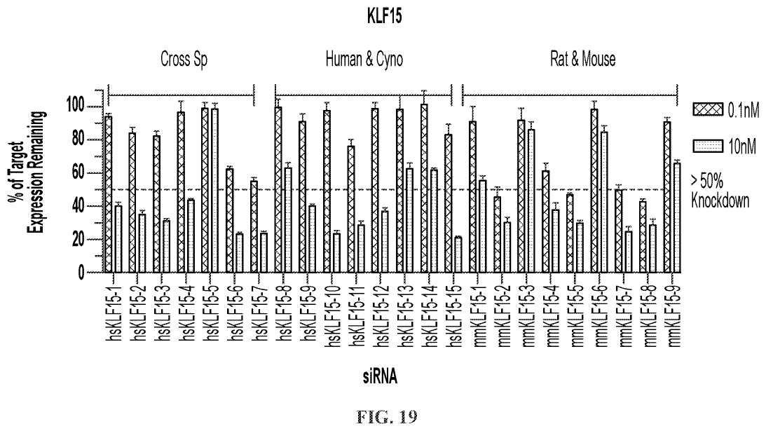

To identify lead oligonucleotide candidates, a panel of oligonucleotide complexes generated to target KLF15 were screened for knockdown of KLF15 expression in cell culture. Expression knockdown was assayed at 10 nM and 0.1 nM. Assayed KLF15 siRNA oligonucleotides were generated with cross-species specificity (i.e., expected to target both human/cynomolgus and mouse/rat KLF15 based on sequence similarity) or specificity for either human/cynomolgus or mouse/rat KLF15 and tested against human or murine KLF15 as appropriate. Candidate oligonucleotides exhibiting the greatest reduction in target expression were subsequently selected for further dose response analysis. FIG. 19 shows KLF15 expression knockdown for screened oligonucleotides.

### Example 24: Knockdown of MED1 Expression with Candidate Oligonucleotides

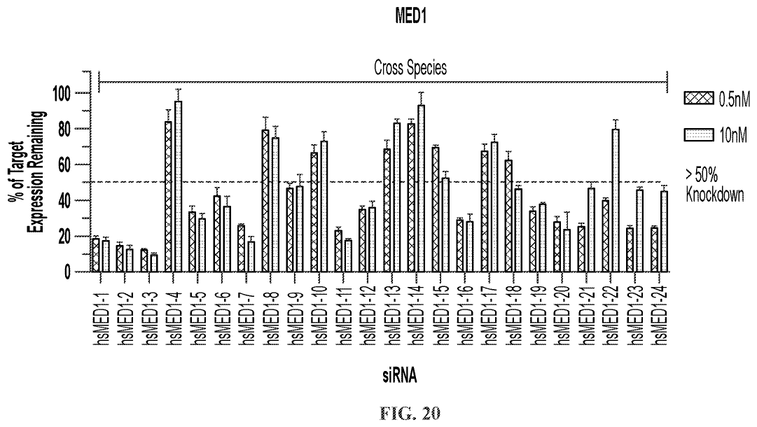

To identify lead oligonucleotide candidates, a panel of oligonucleotide complexes generated to target MED1 were screened for knockdown of MED1 expression in cell culture. Expression knockdown was assayed at 10 nM and 0.5 nM. All assayed MED1 siRNA oligonucleotides were generated with cross-species specificity (i.e., expected to target both human/cynomolgus and mouse/rat MED1 based on sequence similarity) and tested against human MED1. Candidate oligonucleotides exhibiting the greatest reduction in target expression were subsequently selected for further dose response analysis. FIG. 20 shows MED1 expression knockdown for screened oligonucleotides.

### Example 25: Knockdown of MED13 Expression with Candidate Oligonucleotides

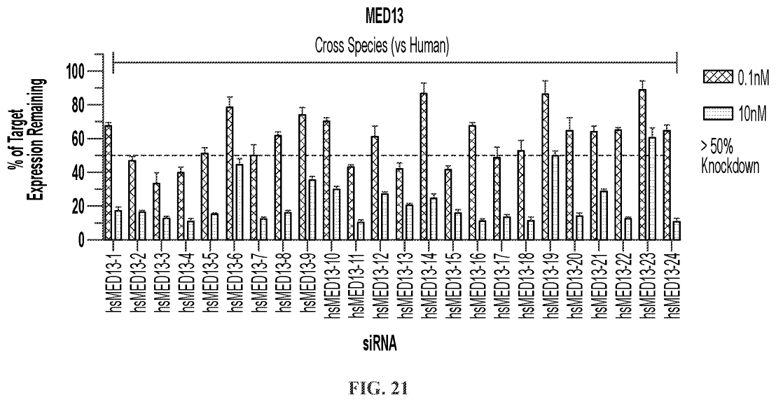

To identify lead oligonucleotide candidates, a panel of oligonucleotide complexes generated to target MED13 were screened for knockdown of MED13 expression in cell culture. Expression knockdown was assayed at 10 nM and 0.1 nM. All assayed MED13 siRNA oligonucleotides were generated with cross-species specificity (i.e., expected to target both human/cynomolgus and mouse/rat MED13 based on sequence similarity) and tested against human MED13. Candidate oligonucleotides exhibiting the greatest reduction in target expression were subsequently selected for further dose response analysis. FIG. 21 shows MED13 expression knockdown for screened oligonucleotides.

### Example 26: Knockdown of PPP1R3A Expression with Candidate Oligonucleotides

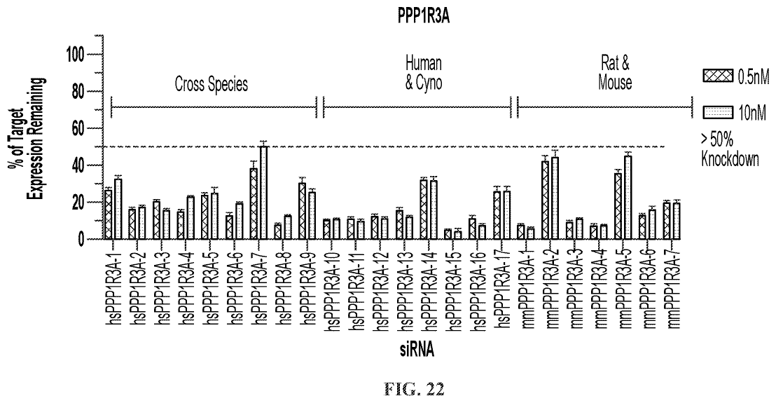

To identify lead oligonucleotide candidates, a panel of oligonucleotide complexes generated to target PPP1R3A were screened for knockdown of PPP1R3A expression in cell culture. Expression knockdown was assayed at 10 nM and 0.5 nM. Assayed PPP1R3A siRNA oligonucleotides were generated with cross-species specificity (i.e., expected to target both human/cynomolgus and mouse/rat PPP1R3A based on sequence similarity) or specificity for either human/cynomolgus or mouse/rat PPP1R3A and tested against human or murine PPP1R3A as appropriate. Candidate oligonucleotides exhibiting the greatest reduction in target expression were subsequently selected for further dose response analysis. FIG. 22 shows PPP1R3A expression knockdown for screened oligonucleotides.

### Example 27: Inhibiting MEF2D with Candidate Oligonucleotides

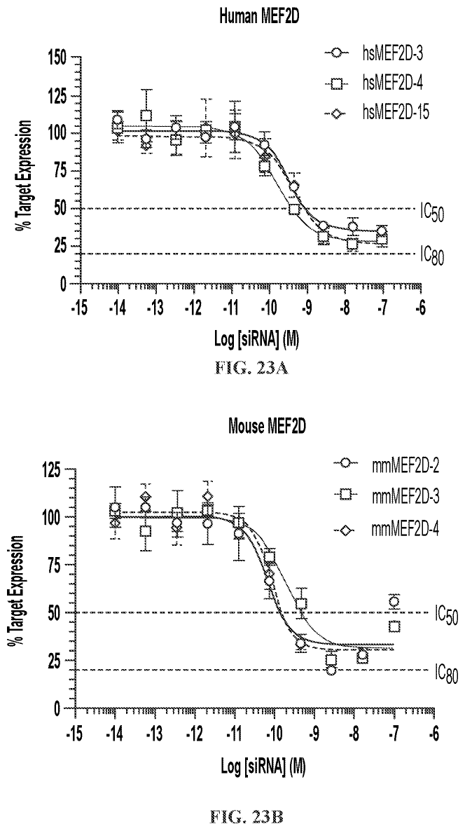

To evaluate candidate oligonucleotides for inhibiting human and murine MEF2D), siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC₅₀ and IC₈₀ values for each oligonucleotide. FIGS. 23A (human) and 23B (murine) show dose response curves for inhibition of MEF2D and Tables 51 (human) and 52 (murine) show the corresponding IC₅₀ values.

TABLE 51
Oligonucleotide inhibition of human MEF2D

| | | | SEQ | | SEQ | | |
|---|---|---|---|---|---|---|---|
| | | Sense | ID | Antisense | ID | IC₅₀ | IC₈₀ |
| Target | siRNA # | Sequence | NO: | Sequence | NO: | (pM) | (pM) |
| Human | hsMEF2D- | mUmGfCmCfUmAfC | 694 | fUfGmGfUmAfAmUf | 718 | 1016 | # |
| MEF2D | 3* | mAfAmCfAmCfAmGf | | CmUfGmUfGmUfUm | | | |
| | | AmUfUmAfCmCfA | | GfUmAfGmGfCmA*f | | | |
| | | | | G*mU | | | |
| | hsMEF2D- | mUmGfAmUfGmAfA | 695 | fGfAmGfAmUfUmGf | 719 | 418 | # |
| | 4* | mGfAmGfUmUfGmAf | | UmCfAmAfCmUfCm | | | |
| | | CmAfAmUfCmUfC | | UfUmCfAmUfCmA*f | | | |
| | | | | G*mG | | | |
| | hsMEF2D- | mAmCfAmAfCmAfC | 706 | fUfCmAfAmCfUmGfG | 730 | 895 | # |
| | 15 | mAfGmAfUmUfAmCf | | mUfAmAfUmCfUmGf | | | |
| | | CmAfGmUfUmGfA | | UmGfUmUfGmU*fA* | | | |
| | | | | mG | | | |

*Oligonucleotide designed to have cross-species activity;
# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

TABLE 52
Oligonucleotide inhibition of murine MEF2D

| | | | SEQ | | SEQ | | |
|---|---|---|---|---|---|---|---|
| | | Sense | ID | Antisense | ID | IC₅₀ | IC₈₀ |
| Target | siRNA # | Sequence | NO: | Sequence | NO: | (pM) | (pM) |
| Murine | mmMEF2D- | mCmCfAmUfGmCfA | 712 | fUfUmGfGmAfUmUf | 736 | 149 | # |
| MEF2D | 2 | mGfUmUfCmAfGmCf | | GmCfUmGfAmAfCm | | | |
| | | AmAfUmCfCmAfA | | UfGmCfAmUfGmG*f | | | |
| | | | | A*mG | | | |
| | mmMEF2D- | mUmUfCmAfGmCfA | 713 | fAfGmAfGmCfUmAfC | 737 | 472 | # |
| | 3 | mAfUmCfCmAfAmGf | | mUfUmGfGmAfUmUf | | | |
| | | UmAfGmCfUmCfU | | GmCfUmGfAmA*fC* | | | |
| | | | | mU | | | |
| | mmMEF2D- | mUmGfGmCfAmAfC | 714 | fUfGmAfGmUfAmAf | 738 | 157 | # |
| | 4 | mAfCmCfAmAfGmUf | | AmCfUmUfGmGfUm | | | |
| | | UmUfAmCfUmCfA | | GfUmUfGmCfCmA*f | | | |
| | | | | C*mG | | | |

# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

### Example 28: Inhibiting KLF15 with Candidate Oligonucleotides

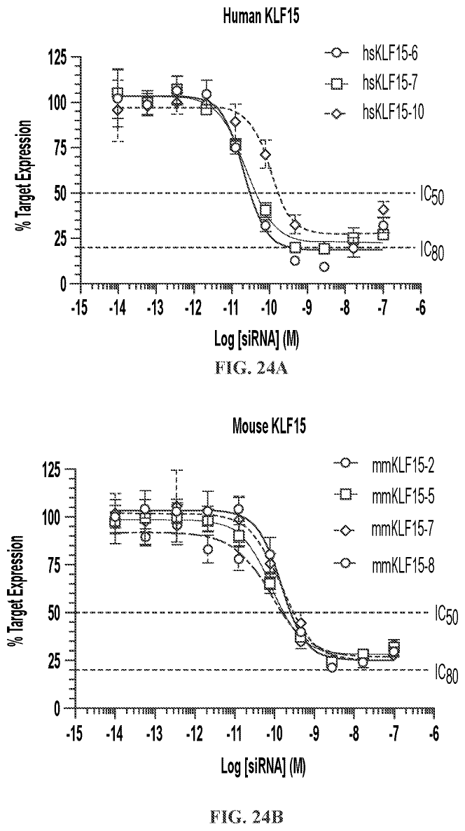

To evaluate candidate oligonucleotides for inhibiting human and murine KLF15, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC₅₀ and IC₈₀ values for each oligonucleotide. FIGS. 24A (human) and 24B (murine) show dose response curves for inhibition of KLF15 and Tables 53 (human) and 54 (murine) show the corresponding IC₅₀ and IC₈₀ values.

TABLE 53
Oligonucleotide inhibition of human KLF15

| | | | SEQ | | SEQ | | |
|---|---|---|---|---|---|---|---|
| | | | ID | Antisense | ID | IC₅₀ | IC₈₀ |
| Target | siRNA # | Sense Sequence | NO: | Sequence | NO: | (pM) | (pM) |
| Human | hsKLF15- | mCmGfUmAfUmAfU | 771 | fAfAmGfGmAfCmCfA | 795 | 30 | 274 |
| KLF15 | 6* | mUfUmUfCmUfUmUf | | mAfAmGfAmAfAmAf | | | |
| | | GmGfUmCfCmUfU | | UmAfUmAfCmG*fU* | | | |
| | | | | mA | | | |
| | hsKLF15- | mAmAfUmAfUmAfU | 772 | fUfAmUfUmGfGmCf | 796 | 42 | # |
| | 7* | mUfUmAfCmUfUmUf | | AmAfAmGfUmAfAm | | | |
| | | GmCfCmAfAmUfA | | AfUmAfUmAfUmU*f | | | |
| | | | | U*mU | | | |
| | hsKLF15- | mAmAfGmCfAmAfU | 775 | fGfAmGfGmCfAmAf | 799 | 162 | # |
| | 10 | mAfAmUfUmUfAmUf | | AmUfAmAfAmUfUm | | | |
| | | UmUfGmCfCmUfC | | AfUmUfGmCfUmU*f | | | |
| | | | | A*mA | | | |

*Oligonucleotide designed to have cross-species activity;
# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

TABLE 54
Oligonucleotide inhibition of murine LKF15

| | | | SEQ | | SEQ | | |
|---|---|---|---|---|---|---|---|
| | | | ID | Antisense | ID | IC₅₀ | IC₈₀ |
| Target | siRNA # | Sense Sequence | NO: | Sequence | NO: | (pM) | (pM) |
| Murine | mmKLF15- | mCmGfCmAfUmCfU | 782 | fAfAmGfAmUfAmUf | 806 | 254 | # |
| KLF15 | 2 | mAfCmAfUmAfAmAf | | UmUfUmAfUmGfUm | | | |
| | | AmUfAmUfCmUfU | | AfGmAfUmGfCmG*f | | | |
| | | | | U*mA | | | |
| | mmKLF15- | mUmAfGmAfAmAfU | 785 | fUfUmCfAmAfUmUf | 809 | 165 | # |
| | 5 | mUfAmGfUmAfCmCf | | GmGfUmAfCmUfAm | | | |
| | | AmAfUmUfGmAfA | | AfUmUfUmCfUmA*f | | | |
| | | | | G*mU | | | |
| | mmKLF15- | mAmCfAmCfGmUfU | 787 | fUfUmUfAmCfAmCfA | 811 | 293 | # |
| | 7 | mGfAmCfAmUfUmUf | | mAfAmUfGmUfCmAf | | | |
| | | GmUfGmUfAmAfA | | AmCfGmUfGmU*fU* | | | |
| | | | | mA | | | |
| | mmKLF15- | mGmAfAmAfUmUfA | 788 | fAfGmUfUmCfAmAf | 812 | 157 | # |
| | 8 | mGfUmAfCmCfAmAf | | UmUfGmGfUmAfCm | | | |
| | | UmUfGmAfAmCfU | | UfAmAfUmUfUmC*f | | | |
| | | | | U*mA | | | |

# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

### Example 29: Inhibiting MED1 with Candidate Oligonucleotides

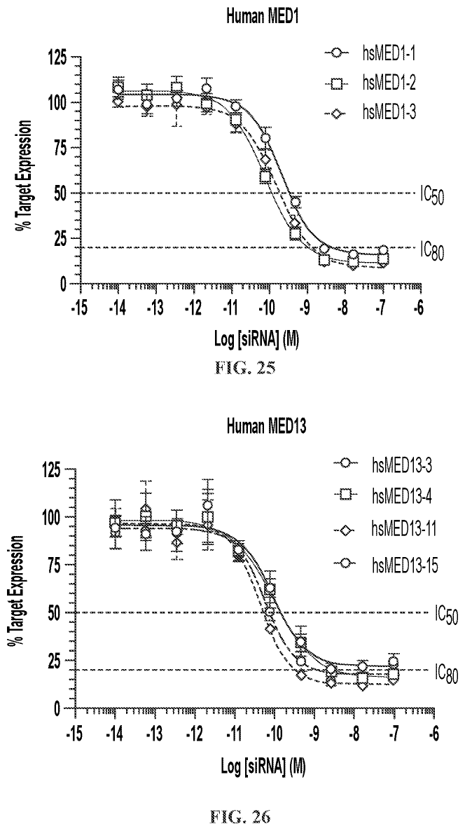

To evaluate candidate oligonucleotides for inhibiting human MED1, siRNAs were screened in a dual luciferase reporter assay. A dose response analysis was conducted over 10 concentrations of each siRNA, from 100 nM to 10 NM, with a fold change of 6 between each dose. Gene inhibition results were used to calculate IC50 and IC80 values for each oligonucleotide. FIG. 25 shows dose response curves for inhibition of MED1 and Table 55 shows the corresponding IC₅₀ and IC₈₀ values.

TABLE 55
Oligonucleotide inhibition of human MED1

| | | | SEQ | | SEQ | | |
|---|---|---|---|---|---|---|---|
| | | | ID | Antisense | ID | IC₅₀ | IC₈₀ |
| Target | siRNA # | Sense Sequence | NO: | Sequence | NO: | (pM) | (pM) |
| Human | hsMED1- | mAmUfGmUfUmAfC | 840 | fAfUmAfUmCfUmGf | 864 | 338 | 3965 |
| MED1 | 1* | mAfUmCfAmCfGmUf | | AmCfGmUfGmAfUm | | | |
| | | CmAfGmAfUmAfU | | GfUmAfAmCfAmU*f | | | |
| | | | | U*mC | | | |
| | hsMED1- | mAmCfAmUfCmAfC | 841 | fAfGmAfAmCfAmUf | 865 | 117 | 1001 |
| | 2* | mGfUmCfAmGfAmUf | | AmUfCmUfGmAfCm | | | |
| | | AmUfGmUfUmCfU | | GfUmGfAmUfGmU*f | | | |
| | | | | A*mA | | | |
| | hsMED1- | mGmUfCmAfGmAfU | 842 | fUfCmCfAmCfAmUfA | 866 | 187 | 1291 |
| | 3* | mAfUmGfUmUfCmUf | | mGfAmAfCmAfUmAf | | | |
| | | AmUfGmUfGmGfA | | UmCfUmGfAmC*fG* | | | |
| | | | | mU | | | |

*Oligonucleotide designed to have cross-species activity;
# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

### Example 30: Inhibiting MED13 with Candidate Oligonucleotides

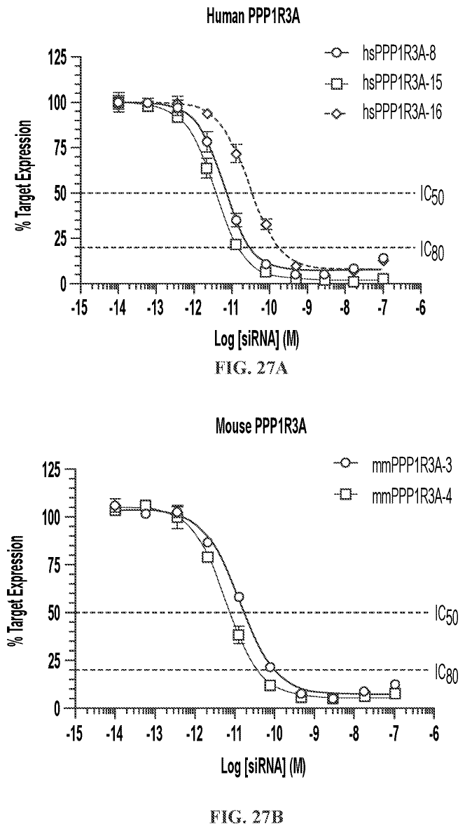

To evaluate candidate oligonucleotides for inhibiting human MED13, siRNAs were screened in a dual luciferase reporter assay. A dose response analysis was conducted over 10 concentrations of each siRNA, from 100 nM to 10 NM, with a fold change of 6 between each dose. Gene inhibition results were used to calculate IC₅₀ and IC₈₀ values for each oligonucleotide. FIG. 26 shows dose response curves for inhibition of MED13 and Table 56 shows the corresponding IC50 and IC₈₀ values.

TABLE 56
Oligonucleotide inhibition of human MED13

| | | | SEQ | | SEQ | | |
|---|---|---|---|---|---|---|---|
| | | | ID | Antisense | ID | IC₅₀ | IC80 |
| Target | siRNA # | Sense Sequence | NO: | Sequence | NO: | (pM) | (pM) |
| Human | hsMED13- | mGmGfGmUfCmAfA | 916 | fAfUmAfCmUfGmAf | 940 | 148 | # |
| MED13 | 3* | mAfUmUfUmUfCmUf | | AmGfAmAfAmAfUm | | | |
| | | UmCfAmGfUmAfU | | UfUmGfAmCfCmC*f | | | |
| | | | | A*mC | | | |
| | hsMED13- | mGmAfAmCfAmAfG | 917 | fAfUmCfAmAfUmUf | 941 | 149 | 3245 |
| | 4* | mAfAmCfCmUfAmAf | | UmUfAmGfGmUfUm | | | |
| | | AmAfUmUfGmAfU | | CfUmUfGmUfUmC*f | | | |
| | | | | U*mG | | | |
| | hsMED13- | mUmCfAmAfCmAfA | 924 | fAfGmUfCmAfAmAf | 948 | 53 | 318 |
| | 11* | mAfUmAfUmUfGmUf | | AmCfAmAfUmAfUm | | | |
| | | UmUfUmGfAmCfU | | UfUmGfUmUfGmA*f | | | |
| | | | | A*mG | | | |
| | hsMED13- | mGmUfCmUfAmAfA | 928 | fAfCmGfAmUfUmAfC | 952 | 77 | 1110 |
| | 15* | mAfUmAfUmAfUmGf | | mAfUmAfUmAfUmUf | | | |
| | | UmAfAmUfCmGfU | | UmUfAmGfAmC*fC* | | | |
| | | | | mA | | | |

*Oligonucleotide designed to have cross-species activity;
# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

### Example 31: Inhibiting PPP1R3A with Candidate Oligonucleotides

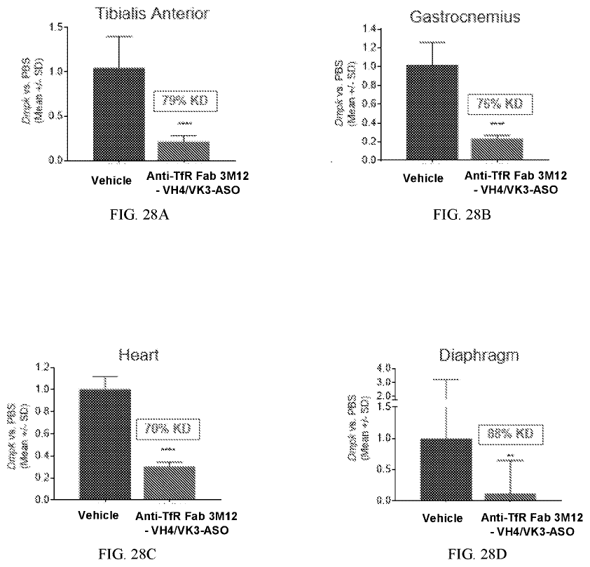

To evaluate candidate oligonucleotides for inhibiting human and murine PPP1R3A, siRNAs were screened in a dual luciferase reporter assay. A dose response analysis was conducted over 10 concentrations of each siRNA, from 100 nM to 10 fM, with a fold change of 6 between each dose. Gene inhibition results were used to calculate IC₅₀ and IC₈₀ values for each oligonucleotide. FIGS. 27A (human) and 27B (murine) show dose response curves for inhibition of PPP1R3A and Tables 57 (human) and 58 (murine) show the corresponding IC₅₀ and IC₈₀.

TABLE 57
Oligonucleotide inhibition of human PPP1R3A

| | | | SEQ | | SEQ | | |
|---|---|---|---|---|---|---|---|
| | | | ID | Antisense | ID | IC₅₀ | IC₈₀ |
| Target | siRNA # | Sense Sequence | NO: | Sequence | NO: | (pM) | (pM) |
| Human | hsPPP1R3A- | mAmCfAmCfAmUfU | 995 | fUfGmCfUmAfAmAf | 1019 | 7 | 29 |
| PPP1R3A | 8* | mAfUmGfAmCfAmUf | | AmUfGmUfCmAfUm | | | |
| | | UmUfUmAfGmCfA | | AfAmUfGmUfGmU*f | | | |
| | | | | C*mU | | | |
| | hsPPP1R3A- | mAmUfCmAfAmGfG | 1002 | fAfAmCfUmCfGmAfA | 1026 | 4 | 14 |
| | 15 | mGfUmAfUmUfAmUf | | mUfAmAfUmAfCmCf | | | |
| | | UmCfGmAfGmUfU | | CmUfUmGfAmU*fA* | | | |
| | | | | mC | | | |
| | hsPPP1R3A- | mUmGfCmUfGmAfU | 1003 | fUfUmGfAmAfUmCfC | 1027 | 34 | 170 |
| | 16 | mUfCmCfUmUfUmGf | | mAfAmAfGmGfAmAf | | | |
| | | GmAfUmUfCmAfA | | UmCfAmGfCmA*fA* | | | |
| | | | | mA | | | |

*Oligonucleotide designed to have cross-species activity;
# Inhibition did not reach 80% or greater at highest concentration tested (100 nM)
‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

TABLE 58
Oligonucleotide inhibition of murine PPP1R3A

| | | | SEQ | | SEQ | | |
|---|---|---|---|---|---|---|---|
| | | | ID | Antisense | ID | IC₅₀ | IC₈₀ |
| Target | siRNA # | Sense Sequence | NO: | Sequence | NO: | (pM) | (pM) |
| Murine | mmPPP1R3A- | mGmAfCmCfAmUfU | 1007 | fAfAmUfGmAfUmCfC | 1031 | 17 | 97 |
| PPP1R3A | 3 | mAfUmGfCmUfUmGf | | mAfAmGfCmAfUmAf | | | |
| | | GmAfUmCfAmUfU | | AmUfGmGfUmC*fC* | | | |
| | | | | mU | | | |
| | mmPPP1R3A- | mAmAfGmGfAmGfA | 1008 | fUfCmCfAmAfUmUfC | 1032 | 8 | 36 |
| | 4 | mAfAmCfAmUfGmGf | | mCfAmUfGmUfUmUf | | | |
| | | AmAfUmUfGmGfA | | CmUfCmCfUmU*fG* | | | |
| | | | | mU | | | |

‘mN’ represents a 2′-O-methyl modified nucleoside (e.g., mU is 2′-O-methyl modified uridine), ‘fN’ represents a 2′-fluoro modified nucleoside (e.g., fU is 2′-fluoro modified uridine), ‘*’ represents a phosphorothioate internucleoside linkage, and lack of “*” between nucleosides indicate phosphodiester internucleoside linkage

### Example 32. In Vivo Activity of Conjugates Containing Anti-TfR1 Fab Conjugated to DMPK-Targeting Oligonucleotide in Mice Expressing the Human TfR1

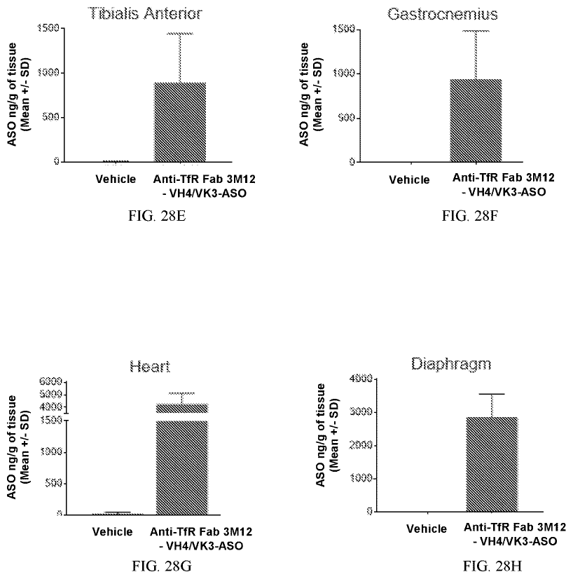

Conjugates containing anti-TfR1 Fab 3M12-VH4/VK3 conjugated to a DMPK-targeting oligonucleotide (ASO300) were tested in a mouse model that expresses human TfR1. The anti-TfR1 Fab 3M12-VH4/VK3 was conjugated to a DMPK-targeting oligonucleotide via a cleavable linker having the structure of Formula (I). The conjugate was administered to the mice at a dose equivalent to 10 mg/kg oligonucleotide on day 0 and day 7. Mice were sacrificed on day 14 and different muscle tissues were collected and analyzed for dmpk mRNA level and oligonucleotide concentration in the tissue. The conjugate reduced mouse wild-type dmpk in Tibialis Anterior by 79% (FIG. 28A), in gastrocnemius by 76% (FIG. 28B), in the heart by 70% (FIG. 28C), and in diaphragm by 88% (FIG. 28D). Oligonucleotide distributions in Tibialis Anterior, gastrocnemius, heart, and diaphragm are shown in FIGS. 28E-28H.

These data indicate that anti-TfR1 Fab 3M12-VH4/VK3 enabled cellular internalization of the conjugate into muscle-specific tissues in an in vivo mouse model, thereby allowing the DMPK-targeting oligonucleotide to reduce expression of DMPK. Similarly, an anti-TfR1 antibody (e.g., anti-TfR1 Fab 3M12-VH4/VK3) can enable cellular internalization of a conjugate containing the anti-TfR1 antibody conjugated to an oligonucleotide targeting MSTN, ACVR1B, INHBA, MLCK1, ACVR1, FBXO32, TRIM63, MEF2D, KLF15, MED1, MED13, or PPP1R3A for reducing expression of MSTN, ACVR1B, INHBA, MLCK1, ACVR1, FBXO32, TRIM63, MEF2D, KLF15, MED1, MED13, or PPP1R3A respectively.

### ADDITIONAL EMBODIMENTS

A1. A complex comprising an anti-transferrin receptor 1 antibody covalently linked to a molecular payload that modulates the expression or activity of myostatin (MSTN), inhibin beta A (INHBA) and/or activin receptor type-1B (ACVR1B), wherein the antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a heavy chain variable region (VH) comprising an amino acid sequence at least 85% identical to SEQ ID NO: 71; and/or a light chain variable region (VL) comprising an amino acid sequence at least 85% identical to SEQ ID NO: 70;
(iii) a heavy chain variable region (VH) comprising an amino acid sequence at least 85% identical to SEQ ID NO: 72; and/or a light chain variable region (VL) comprising an amino acid sequence at least 85% identical to SEQ ID NO: 70;
(iv) a heavy chain variable region (VH) comprising an amino acid sequence at least 85% identical to SEQ ID NO: 73; and/or a light chain variable region (VL) comprising an amino acid sequence at least 85% identical to SEQ ID NO: 74;
(v) a heavy chain variable region (VH) comprising an amino acid sequence at least 85% identical to SEQ ID NO: 73; and/or a light chain variable region (VL) comprising an amino acid sequence at least 85% identical to SEQ ID NO: 75;
(vi) a heavy chain variable region (VH) comprising an amino acid sequence at least 85% identical to SEQ ID NO: 76; and/or a light chain variable region (VL) comprising an amino acid sequence at least 85% identical to SEQ ID NO: 74;
(vii) a heavy chain variable region (VH) comprising an amino acid sequence at least 85% identical to SEQ ID NO: 69; and/or a light chain variable region (VL) comprising an amino acid sequence at least 85% identical to SEQ ID NO: 70;
(viii) a heavy chain variable region (VH) comprising an amino acid sequence at least 85% identical to SEQ ID NO: 77; and/or a light chain variable region (VL) comprising an amino acid sequence at least 85% identical to SEQ ID NO: 78;
(ix) a heavy chain variable region (VH) comprising an amino acid sequence at least 85% identical to SEQ ID NO: 79; and/or a light chain variable region (VL) comprising an amino acid sequence at least 85% identical to SEQ ID NO: 80; or
(x) a heavy chain variable region (VH) comprising an amino acid sequence at least 85% identical to SEQ ID NO: 77; and/or a light chain variable region (VL) comprising an amino acid sequence at least 85% identical to SEQ ID NO: 80.
A2. The complex of embodiment A1, wherein the antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 7land a VL comprising the amino acid sequence of SEQ ID NO: 70;
(iii) a VH comprising the amino acid sequence of SEQ ID NO: 72 and a VL comprising the amino acid sequence of SEQ ID NO: 70;
(iv) a VH comprising the amino acid sequence of SEQ ID NO: 73 and a VL comprising the amino acid sequence of SEQ ID NO: 74;
(v) a VH comprising the amino acid sequence of SEQ ID NO: 73 and a VL comprising the amino acid sequence of SEQ ID NO: 75;
(vi) a VH comprising the amino acid sequence of SEQ ID NO: 76 and a VL comprising the amino acid sequence of SEQ ID NO: 74;
(vii) a VH comprising the amino acid sequence of SEQ ID NO: 69 and a VL comprising the amino acid sequence of SEQ ID NO: 70;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
A3. The complex of embodiment A1 or embodiment A2, wherein the antibody is selected from the group consisting of a full-length IgG, a Fab fragment, a Fab′ fragment, a F(ab′)2 fragment, a scFv, and a Fv.
A4. The complex of embodiment A3, wherein the antibody is a full-length IgG, optionally wherein the full-length IgG comprises a heavy chain constant region of the isotype IgG1, IgG2, IgG3, or IgG4.
A5. The complex of embodiment A4, wherein the antibody comprises:
(i) a heavy chain comprising an amino acid sequence at least 85% identical to SEQ ID NO: 91; and/or a light chain comprising an amino acid sequence at least 85% identical to SEQ ID NO: 90;
(ii) a heavy chain comprising an amino acid sequence at least 85% identical to SEQ ID NO: 86; and/or a light chain comprising an amino acid sequence at least 85% identical to SEQ ID NO: 85;
(iii) a heavy chain comprising an amino acid sequence at least 85% identical to SEQ ID NO: 87; and/or a light chain comprising an amino acid sequence at least 85% identical to SEQ ID NO: 85;
(iv) a heavy chain comprising an amino acid sequence at least 85% identical to SEQ ID NO: 88; and/or a light chain comprising an amino acid sequence at least 85% identical to SEQ ID NO: 89;
(v) a heavy chain comprising an amino acid sequence at least 85% identical to SEQ ID NO: 88; and/or a light chain comprising an amino acid sequence at least 85% identical to SEQ ID NO: 90;
(vi) a heavy chain comprising an amino acid sequence at least 85% identical to SEQ ID NO: 91; and/or a light chain comprising an amino acid sequence at least 85% identical to SEQ ID NO: 89;
(vii) a heavy chain comprising an amino acid sequence at least 85% identical to SEQ ID NO: 84; and/or a light chain comprising an amino acid sequence at least 85% identical to SEQ ID NO: 85;
(viii) a heavy chain comprising an amino acid sequence at least 85% identical to SEQ ID NO: 92; and/or a light chain comprising an amino acid sequence at least 85% identical to SEQ ID NO: 93;
(ix) a heavy chain comprising an amino acid sequence at least 85% identical to SEQ ID NO: 94; and/or a light chain comprising an amino acid sequence at least 85% identical to SEQ ID NO: 95; or
(x) a heavy chain comprising an amino acid sequence at least 85% identical to SEQ ID NO: 92; and/or a light chain comprising an amino acid sequence at least 85% identical to SEQ ID NO: 95.
A6. The complex of embodiment A3, wherein the antibody is a Fab fragment.
A7. The complex of embodiment A6, wherein the antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 98; and/or a light chain comprising an amino acid sequence at least 85% identical to SEQ ID NO: 85;
(iii) a heavy chain comprising an amino acid sequence at least 85% identical to SEQ ID NO: 99; and/or a light chain comprising an amino acid sequence at least 85% identical to SEQ ID NO: 85;
(iv) a heavy chain comprising an amino acid sequence at least 85% identical to SEQ ID NO: 100; and/or a light chain comprising an amino acid sequence at least 85% identical to SEQ ID NO: 89;
(v) a heavy chain comprising an amino acid sequence at least 85% identical to SEQ ID NO: 100; and/or a light chain comprising an amino acid sequence at least 85% identical to SEQ ID NO: 90;
(vi) a heavy chain comprising an amino acid sequence at least 85% identical to SEQ ID NO: 101; and/or a light chain comprising an amino acid sequence at least 85% identical to SEQ ID NO: 89;
(vii) a heavy chain comprising an amino acid sequence at least 85% identical to SEQ ID NO: 97; and/or a light chain comprising an amino acid sequence at least 85% identical to SEQ ID NO: 85;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
A8. The complex of embodiment A6 or embodiment A7, wherein the antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 98; and a light chain comprising the amino acid sequence of SEQ ID NO: 85;
(iii) a heavy chain comprising the amino acid sequence of SEQ ID NO: 99; and a light chain comprising the amino acid sequence of SEQ ID NO: 85;
(iv) a heavy chain comprising the amino acid sequence of SEQ ID NO: 100; and a light chain comprising the amino acid sequence of SEQ ID NO: 89;
(v) a heavy chain comprising the amino acid sequence of SEQ ID NO: 100; and a light chain comprising the amino acid sequence of SEQ ID NO: 90;
(vi) a heavy chain comprising the amino acid sequence of SEQ ID NO: 101; and a light chain comprising the amino acid sequence of SEQ ID NO: 89;
(vii) a heavy chain comprising the amino acid sequence of SEQ ID NO: 97; and a light chain comprising the amino acid sequence of SEQ ID NO: 85;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
A9. The complex of any one of embodiments A1 to A8, wherein the equilibrium dissociation constant (K_{D}) of binding of the antibody to the transferrin receptor is in a range from 10⁻¹¹ M to 10⁻⁶ M.
A10. The complex of any one of embodiments A1 to A9, wherein the antibody does not specifically bind to the transferrin binding site of the transferrin receptor and/or wherein the antibody does not inhibit binding of transferrin to the transferrin receptor.
A11. The complex of any one of embodiments A1 to A10, wherein the antibody is cross-reactive with extracellular epitopes of two or more of a human, non-human primate and rodent transferrin receptor.
A12. The complex of any one of embodiments A1 to A11, wherein the complex is configured to promote transferrin receptor mediated internalization of the molecular payload into a muscle cell.
A13. The complex of any one of embodiments A1 to A12, wherein the molecular payload is an oligonucleotide comprising an antisense strand comprising a region of complementarity to an MSTN target sequence, optionally wherein the MSTN target sequence is an MSTN mRNA sequence as set forth in SEQ ID NOs: 146-148, or an MSTN target sequence as set forth in any one of SEQ ID NOs: 149-196, further optionally wherein the antisense strand is 18-25 nucleotides in length and/or the region of complementarity is at least 16 nucleosides in length.
A14. The complex of embodiments A13, wherein the antisense strand comprises at least 16 consecutive nucleotides of a nucleotide sequence set forth in any one of SEQ ID NOs: 197-220, wherein each of the Us are optionally and independently Ts, optionally wherein the antisense strand comprises the nucleotide sequence of any one of SEQ ID NOs: 197-220, wherein each of the Us are optionally and independently Ts.
A15. The complex of any one of embodiments A1 to A12, wherein the molecular payload is an oligonucleotide comprising an antisense strand comprising a region of complementarity to an INHBA target sequence, optionally wherein the INHBA target sequence is an INHBA mRNA sequence as set forth in SEQ ID NO: 269 or SEQ ID NO: 270, or an INHBA target sequence as set forth in any one of SEQ ID NOs: 271-318, further optionally wherein the antisense strand is 18-25 nucleotides in length and/or the region of complementarity is at least 16 nucleosides in length.
A16. The complex of embodiment A15, wherein the antisense strand comprises at least 16 consecutive nucleotides of a nucleotide sequence set forth in any one of SEQ ID NOs: 319-342, wherein each of the Us are optionally and independently Ts, optionally wherein the antisense strand comprises the nucleotide sequence of any one of SEQ ID NOs: 319-342, wherein each of the Us are optionally and independently Ts.
A17. The complex of any one of embodiments A1 to A12, wherein the molecular payload is an oligonucleotide comprising an antisense strand comprising a region of complementarity to an ACVR1B target sequence, optionally wherein the ACVR1B target sequence is an ACVR1B mRNA sequence as set forth in any one of SEQ ID NOs: 367-370, or an ACVR1B target sequence as set forth in any one of SEQ ID NOs: 221-268, further optionally wherein the antisense strand is 18-25 nucleotides in length and/or the region of complementarity is at least 16 nucleosides in length.
A18. The complex of embodiment A17, wherein the antisense strand comprises at least 16 consecutive nucleotides of a nucleotide sequence set forth in any one of SEQ ID NOs: 343-366, wherein each of the Us are optionally and independently Ts, optionally wherein the antisense strand comprises the nucleotide sequence of any one of SEQ ID NOs: 343-366, wherein each of the Us are optionally and independently Ts.
A19. The complex of any one of embodiments A13 to A18, wherein the oligonucleotide further comprises a sense strand that hybridizes to the antisense strand to form a double stranded siRNA.
A20. The complex of any one of embodiments A13 to A19, wherein the oligonucleotide comprises one or more modified nucleosides, optionally wherein each nucleoside in the oligonucleotide is a modified nucleoside.
A21. The complex of embodiment A20, wherein the one or more modified nucleosides are 2′ modified nucleotides, optionally wherein the one or more 2′ modified nucleosides are selected from: 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethyl-bridged nucleic acid (cEt), optionally wherein the 2′ modified nucleotide is 2′-O-methyl or 2′-fluoro (2′-F).
A22. The complex of any one of embodiments A13 to A21, wherein the oligonucleotide comprises one or more phosphorothioate internucleoside linkages, optionally wherein the one or more phosphorothioate internucleoside linkage are present on the antisense strand of the RNAi oligonucleotide, further optionally wherein the two internucleoside linkages at the 3′ end of the sense strands are phosphorothioate internucleoside linkages.
A23. The complex of any one of embodiments A13, A14, and A19 to A22, wherein the oligonucleotide is an siRNA listed in Table 10.
A24. The complex of any one of embodiments A15, A16, and A19 to A22, wherein the oligonucleotide is an siRNA listed in Table 13.
A25. The complex of any one of embodiments A17 to A22, wherein the oligonucleotide is an siRNA listed in Table 16.
A26. The complex of any one of embodiments A1 to A25, wherein the antibody is covalently linked to the molecular payload via
(i) a cleavable linker, optionally wherein the cleavable linker comprises a valine-citrulline sequence; or
(ii) a non-cleavable linker, optionally wherein the non-cleavable linker is an alkane linker.
A27. A method of reducing MSTN, INHBA, and/or ACVR1B expression in a muscle cell, the method comprising contacting the muscle cell with an effective amount of the complex of any one of embodiments A1 to A26 for promoting internalization of the molecular payload to the muscle cell.
A28. A method of treating muscle atrophy the method comprising administering to a subject in need thereof an effective amount of the complex of any one of embodiments A1 to A26, wherein the subject has elevated expression or activity of MSTN, INHBA, and/or ACVR1B.
A29. The method of embodiment A27 or embodiment A28, wherein the complex reduces RNA level of MSTN, INHBA, and/or ACVR1B.
A30. The method of any one of embodiments A27 to A29, wherein the complex reduces protein level of MSTN, INHBA, and/or ACVR1B.
B1. A complex comprising an anti-transferrin receptor 1 antibody covalently linked to a molecular payload that modulates the expression or activity of myosin light chain kinase 1 (MLCK1), wherein the antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a heavy chain variable region (VH) comprising an amino acid sequence at least 85% identical to SEQ ID NO: 71; and/or a light chain variable region (VL) comprising an amino acid sequence at least 85% identical to SEQ ID NO: 70;
(iii) a heavy chain variable region (VH) comprising an amino acid sequence at least 85% identical to SEQ ID NO: 72; and/or a light chain variable region (VL) comprising an amino acid sequence at least 85% identical to SEQ ID NO: 70;
(iv) a heavy chain variable region (VH) comprising an amino acid sequence at least 85% identical to SEQ ID NO: 73; and/or a light chain variable region (VL) comprising an amino acid sequence at least 85% identical to SEQ ID NO: 74;
(v) a heavy chain variable region (VH) comprising an amino acid sequence at least 85% identical to SEQ ID NO: 73; and/or a light chain variable region (VL) comprising an amino acid sequence at least 85% identical to SEQ ID NO: 75;
(vi) a heavy chain variable region (VH) comprising an amino acid sequence at least 85% identical to SEQ ID NO: 76; and/or a light chain variable region (VL) comprising an amino acid sequence at least 85% identical to SEQ ID NO: 74;
(vii) a heavy chain variable region (VH) comprising an amino acid sequence at least 85% identical to SEQ ID NO: 69; and/or a light chain variable region (VL) comprising an amino acid sequence at least 85% identical to SEQ ID NO: 70;
(viii) a heavy chain variable region (VH) comprising an amino acid sequence at least 85% identical to SEQ ID NO: 77; and/or a light chain variable region (VL) comprising an amino acid sequence at least 85% identical to SEQ ID NO: 78;
(ix) a heavy chain variable region (VH) comprising an amino acid sequence at least 85% identical to SEQ ID NO: 79; and/or a light chain variable region (VL) comprising an amino acid sequence at least 85% identical to SEQ ID NO: 80; or
(x) a heavy chain variable region (VH) comprising an amino acid sequence at least 85% identical to SEQ ID NO: 77; and/or a light chain variable region (VL) comprising an amino acid sequence at least 85% identical to SEQ ID NO: 80.
B2. The complex of embodiment B1, wherein the antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 7land a VL comprising the amino acid sequence of SEQ ID NO: 70;
(iii) a VH comprising the amino acid sequence of SEQ ID NO: 72 and a VL comprising the amino acid sequence of SEQ ID NO: 70;
(iv) a VH comprising the amino acid sequence of SEQ ID NO: 73 and a VL comprising the amino acid sequence of SEQ ID NO: 74;
(v) a VH comprising the amino acid sequence of SEQ ID NO: 73 and a VL comprising the amino acid sequence of SEQ ID NO: 75;
(vi) a VH comprising the amino acid sequence of SEQ ID NO: 76 and a VL comprising the amino acid sequence of SEQ ID NO: 74;
(vii) a VH comprising the amino acid sequence of SEQ ID NO: 69 and a VL comprising the amino acid sequence of SEQ ID NO: 70;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
B3. The complex of embodiment B1 or embodiment B2, wherein the antibody is selected from the group consisting of a full-length IgG, a Fab fragment, a Fab′ fragment, a F(ab′)2 fragment, a scFv, and a Fv.
B4. The complex of embodiment B3, wherein the antibody is a full-length IgG, optionally wherein the full-length IgG comprises a heavy chain constant region of the isotype IgG1, IgG2, IgG3, or IgG4.
B5. The complex of embodiment B4, wherein the antibody comprises:
(i) a heavy chain comprising an amino acid sequence at least 85% identical to SEQ ID NO: 91; and/or a light chain comprising an amino acid sequence at least 85% identical to SEQ ID NO: 90;
(ii) a heavy chain comprising an amino acid sequence at least 85% identical to SEQ ID NO: 86; and/or a light chain comprising an amino acid sequence at least 85% identical to SEQ ID NO: 85;
(iii) a heavy chain comprising an amino acid sequence at least 85% identical to SEQ ID NO: 87; and/or a light chain comprising an amino acid sequence at least 85% identical to SEQ ID NO: 85;
(iv) a heavy chain comprising an amino acid sequence at least 85% identical to SEQ ID NO: 88; and/or a light chain comprising an amino acid sequence at least 85% identical to SEQ ID NO: 89;
(v) a heavy chain comprising an amino acid sequence at least 85% identical to SEQ ID NO: 88; and/or a light chain comprising an amino acid sequence at least 85% identical to SEQ ID NO: 90;
(vi) a heavy chain comprising an amino acid sequence at least 85% identical to SEQ ID NO: 91; and/or a light chain comprising an amino acid sequence at least 85% identical to SEQ ID NO: 89;
(vii) a heavy chain comprising an amino acid sequence at least 85% identical to SEQ ID NO: 84; and/or a light chain comprising an amino acid sequence at least 85% identical to SEQ ID NO: 85;
(viii) a heavy chain comprising an amino acid sequence at least 85% identical to SEQ ID NO: 92; and/or a light chain comprising an amino acid sequence at least 85% identical to SEQ ID NO: 93;
(ix) a heavy chain comprising an amino acid sequence at least 85% identical to SEQ ID NO: 94; and/or a light chain comprising an amino acid sequence at least 85% identical to SEQ ID NO: 95; or
(x) a heavy chain comprising an amino acid sequence at least 85% identical to SEQ ID NO: 92; and/or a light chain comprising an amino acid sequence at least 85% identical to SEQ ID NO: 95.
B6. The complex of embodiment B3, wherein the antibody is a Fab fragment.
B7. The complex of embodiment B6, wherein the antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 98; and/or a light chain comprising an amino acid sequence at least 85% identical to SEQ ID NO: 85;
(iii) a heavy chain comprising an amino acid sequence at least 85% identical to SEQ ID NO: 99; and/or a light chain comprising an amino acid sequence at least 85% identical to SEQ ID NO: 85;
(iv) a heavy chain comprising an amino acid sequence at least 85% identical to SEQ ID NO: 100; and/or a light chain comprising an amino acid sequence at least 85% identical to SEQ ID NO: 89;
(v) a heavy chain comprising an amino acid sequence at least 85% identical to SEQ ID NO: 100; and/or a light chain comprising an amino acid sequence at least 85% identical to SEQ ID NO: 90;
(vi) a heavy chain comprising an amino acid sequence at least 85% identical to SEQ ID NO: 101; and/or a light chain comprising an amino acid sequence at least 85% identical to SEQ ID NO: 89;
(vii) a heavy chain comprising an amino acid sequence at least 85% identical to SEQ ID NO: 97; and/or a light chain comprising an amino acid sequence at least 85% identical to SEQ ID NO: 85;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
B8. The complex of embodiment B6 or embodiment B7, wherein the antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 98; and a light chain comprising the amino acid sequence of SEQ ID NO: 85;
(iii) a heavy chain comprising the amino acid sequence of SEQ ID NO: 99; and a light chain comprising the amino acid sequence of SEQ ID NO: 85;
(iv) a heavy chain comprising the amino acid sequence of SEQ ID NO: 100; and a light chain comprising the amino acid sequence of SEQ ID NO: 89;
(v) a heavy chain comprising the amino acid sequence of SEQ ID NO: 100; and a light chain comprising the amino acid sequence of SEQ ID NO: 90;
(vi) a heavy chain comprising the amino acid sequence of SEQ ID NO: 101; and a light chain comprising the amino acid sequence of SEQ ID NO: 89;
(vii) a heavy chain comprising the amino acid sequence of SEQ ID NO: 97; and a light chain comprising the amino acid sequence of SEQ ID NO: 85;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
B9. The complex of any one of embodiments B1 to B8, wherein the equilibrium dissociation constant (K_{D}) of binding of the antibody to the transferrin receptor is in a range from 10⁻¹¹ M to 10⁻⁶ M.
B10. The complex of any one of embodiments B1 to B9, wherein the antibody does not specifically bind to the transferrin binding site of the transferrin receptor and/or wherein the antibody does not inhibit binding of transferrin to the transferrin receptor.
B11. The complex of any one of embodiments B1 to B10, wherein the antibody is cross-reactive with extracellular epitopes of two or more of a human, non-human primate and rodent transferrin receptor.
B12. The complex of any one of embodiments B1 to B11, wherein the complex is configured to promote transferrin receptor mediated internalization of the molecular payload into a muscle cell.
B13. The complex of any one of embodiments B1 to B12, wherein the molecular payload is an oligonucleotide.
B14. The complex of any one of embodiments B1 to B13, wherein the molecular payload is an oligonucleotide comprising an antisense strand comprising a region of complementarity to MLCK1 mRNA, optionally wherein the MLCK1 mRNA is set forth in SEQ ID NO: 152.
B15. The complex of embodiment B14, wherein the antisense strand is 18-25 nucleotides in length and/or the region of complementarity is at least 16 nucleosides in length.
B16. The complex of embodiment B14 or embodiment B15, wherein the oligonucleotide further comprises a sense strand that hybridizes to the antisense strand to form a double stranded siRNA.
B17. The complex of any one of embodiments B14 to B16, wherein the oligonucleotide comprises one or more modified nucleosides, optionally wherein each nucleoside in the oligonucleotide is a modified nucleoside.
B18. The complex of embodiment B17, wherein the one or more modified nucleosides are 2′ modified nucleotides, optionally wherein the one or more 2′ modified nucleosides are selected from: 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethyl-bridged nucleic acid (cEt).
B19. The complex of embodiment B18, wherein the 2′ modified nucleotide is 2′-O-methyl or 2′-fluoro (2′-F).
B20. The complex of any one of embodiments B14 to B19, wherein the oligonucleotide comprises one or more phosphorothioate internucleoside linkages.
B21. The complex of any one of embodiments B1 to B20, wherein the antibody is covalently linked to the molecular payload via
(i) a cleavable linker, optionally wherein the cleavable linker comprises a valine-citrulline sequence; or
(ii) a non-cleavable linker, optionally wherein the non-cleavable linker is an alkane linker.
B22. A method of reducing MLCK1 expression in a muscle cell, the method comprising contacting the muscle cell with an effective amount of the complex of any one of embodiments B1 to B21 for promoting internalization of the oligonucleotide to the muscle cell.
B23. A method of treating irritable bowel syndrome (IBS) or irritable bowel disease (IBD) the method comprising administering to a subject in need thereof an effective amount of the complex of any one of embodiment B1 to B21, wherein the subject has elevated levels of MLCK1 protein, optionally wherein the subject is human, and optionally wherein the administration is intravenous.
B24. The method of embodiment B22 or embodiment B23, wherein the complex reduces MLCK1 RNA level.
B25. The method of any one of embodiments B22 to B24, wherein the complex reduces MLCK1 protein level.
C1. A complex comprising an anti-transferrin receptor 1 antibody covalently linked to a molecular payload that modulates the expression or activity of Activin A receptor, type 1 (ACVR1), wherein the antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a heavy chain variable region (VH) comprising an amino acid sequence at least 85% identical to SEQ ID NO: 71; and/or a light chain variable region (VL) comprising an amino acid sequence at least 85% identical to SEQ ID NO: 70;
(iii) a heavy chain variable region (VH) comprising an amino acid sequence at least 85% identical to SEQ ID NO: 72; and/or a light chain variable region (VL) comprising an amino acid sequence at least 85% identical to SEQ ID NO: 70;
(iv) a heavy chain variable region (VH) comprising an amino acid sequence at least 85% identical to SEQ ID NO: 73; and/or a light chain variable region (VL) comprising an amino acid sequence at least 85% identical to SEQ ID NO: 74;
(v) a heavy chain variable region (VH) comprising an amino acid sequence at least 85% identical to SEQ ID NO: 73; and/or a light chain variable region (VL) comprising an amino acid sequence at least 85% identical to SEQ ID NO: 75;
(vi) a heavy chain variable region (VH) comprising an amino acid sequence at least 85% identical to SEQ ID NO: 76; and/or a light chain variable region (VL) comprising an amino acid sequence at least 85% identical to SEQ ID NO: 74;
(vii) a heavy chain variable region (VH) comprising an amino acid sequence at least 85% identical to SEQ ID NO: 69; and/or a light chain variable region (VL) comprising an amino acid sequence at least 85% identical to SEQ ID NO: 70;
(viii) a heavy chain variable region (VH) comprising an amino acid sequence at least 85% identical to SEQ ID NO: 77; and/or a light chain variable region (VL) comprising an amino acid sequence at least 85% identical to SEQ ID NO: 78;
(ix) a heavy chain variable region (VH) comprising an amino acid sequence at least 85% identical to SEQ ID NO: 79; and/or a light chain variable region (VL) comprising an amino acid sequence at least 85% identical to SEQ ID NO: 80; or
(x) a heavy chain variable region (VH) comprising an amino acid sequence at least 85% identical to SEQ ID NO: 77; and/or a light chain variable region (VL) comprising an amino acid sequence at least 85% identical to SEQ ID NO: 80.
C2. The complex of embodiment C1, wherein the antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 7land a VL comprising the amino acid sequence of SEQ ID NO: 70;
(iii) a VH comprising the amino acid sequence of SEQ ID NO: 72 and a VL comprising the amino acid sequence of SEQ ID NO: 70;
(iv) a VH comprising the amino acid sequence of SEQ ID NO: 73 and a VL comprising the amino acid sequence of SEQ ID NO: 74;
(v) a VH comprising the amino acid sequence of SEQ ID NO: 73 and a VL comprising the amino acid sequence of SEQ ID NO: 75;
(vi) a VH comprising the amino acid sequence of SEQ ID NO: 76 and a VL comprising the amino acid sequence of SEQ ID NO: 74;
(vii) a VH comprising the amino acid sequence of SEQ ID NO: 69 and a VL comprising the amino acid sequence of SEQ ID NO: 70;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
C3. The complex of embodiment C1 or embodiment C2, wherein the antibody is selected from the group consisting of a full-length IgG, a Fab fragment, a Fab′ fragment, a F(ab′)2 fragment, a scFv, and a Fv.
C4. The complex of embodiment C3, wherein the antibody is a full-length IgG, optionally wherein the full-length IgG comprises a heavy chain constant region of the isotype IgG1, IgG2, IgG3, or IgG4.
C5. The complex of embodiment C4, wherein the antibody comprises:
(i) a heavy chain comprising an amino acid sequence at least 85% identical to SEQ ID NO: 91; and/or a light chain comprising an amino acid sequence at least 85% identical to SEQ ID NO: 90;
(ii) a heavy chain comprising an amino acid sequence at least 85% identical to SEQ ID NO: 86; and/or a light chain comprising an amino acid sequence at least 85% identical to SEQ ID NO: 85;
(iii) a heavy chain comprising an amino acid sequence at least 85% identical to SEQ ID NO: 87; and/or a light chain comprising an amino acid sequence at least 85% identical to SEQ ID NO: 85;
(iv) a heavy chain comprising an amino acid sequence at least 85% identical to SEQ ID NO: 88; and/or a light chain comprising an amino acid sequence at least 85% identical to SEQ ID NO: 89;
(v) a heavy chain comprising an amino acid sequence at least 85% identical to SEQ ID NO: 88; and/or a light chain comprising an amino acid sequence at least 85% identical to SEQ ID NO: 90;
(vi) a heavy chain comprising an amino acid sequence at least 85% identical to SEQ ID NO: 91; and/or a light chain comprising an amino acid sequence at least 85% identical to SEQ ID NO: 89;
(vii) a heavy chain comprising an amino acid sequence at least 85% identical to SEQ ID NO: 84; and/or a light chain comprising an amino acid sequence at least 85% identical to SEQ ID NO: 85;
(viii) a heavy chain comprising an amino acid sequence at least 85% identical to SEQ ID NO: 92; and/or a light chain comprising an amino acid sequence at least 85% identical to SEQ ID NO: 93;
(ix) a heavy chain comprising an amino acid sequence at least 85% identical to SEQ ID NO: 94; and/or a light chain comprising an amino acid sequence at least 85% identical to SEQ ID NO: 95; or
(x) a heavy chain comprising an amino acid sequence at least 85% identical to SEQ ID NO: 92; and/or a light chain comprising an amino acid sequence at least 85% identical to SEQ ID NO: 95.
C6. The complex of embodiment C3, wherein the antibody is a Fab fragment.
C7. The complex of embodiment C6, wherein the antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 98; and/or a light chain comprising an amino acid sequence at least 85% identical to SEQ ID NO: 85;
(iii) a heavy chain comprising an amino acid sequence at least 85% identical to SEQ ID NO: 99; and/or a light chain comprising an amino acid sequence at least 85% identical to SEQ ID NO: 85;
(iv) a heavy chain comprising an amino acid sequence at least 85% identical to SEQ ID NO: 100; and/or a light chain comprising an amino acid sequence at least 85% identical to SEQ ID NO: 89;
(v) a heavy chain comprising an amino acid sequence at least 85% identical to SEQ ID NO: 100; and/or a light chain comprising an amino acid sequence at least 85% identical to SEQ ID NO: 90;
(vi) a heavy chain comprising an amino acid sequence at least 85% identical to SEQ ID NO: 101; and/or a light chain comprising an amino acid sequence at least 85% identical to SEQ ID NO: 89;
(vii) a heavy chain comprising an amino acid sequence at least 85% identical to SEQ ID NO: 97; and/or a light chain comprising an amino acid sequence at least 85% identical to SEQ ID NO: 85;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
C8. The complex of embodiment C6 or embodiment C7, wherein the antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 98; and a light chain comprising the amino acid sequence of SEQ ID NO: 85;
(iii) a heavy chain comprising the amino acid sequence of SEQ ID NO: 99; and a light chain comprising the amino acid sequence of SEQ ID NO: 85;
(iv) a heavy chain comprising the amino acid sequence of SEQ ID NO: 100; and a light chain comprising the amino acid sequence of SEQ ID NO: 89;
(v) a heavy chain comprising the amino acid sequence of SEQ ID NO: 100; and a light chain comprising the amino acid sequence of SEQ ID NO: 90;
(vi) a heavy chain comprising the amino acid sequence of SEQ ID NO: 97; and a light chain comprising the amino acid sequence of SEQ ID NO: 85;
(vii) a heavy chain comprising the amino acid sequence of SEQ ID NO: 101; and a light chain comprising the amino acid sequence of SEQ ID NO: 90;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
C9. The complex of any one of embodiments C1 to C8, wherein the equilibrium dissociation constant (K_{D}) of binding of the antibody to the transferrin receptor is in a range from 10⁻¹¹ M to 10⁻⁶ M.
C10. The complex of any one of embodiments C1 to C9, wherein the antibody does not specifically bind to the transferrin binding site of the transferrin receptor and/or wherein the antibody does not inhibit binding of transferrin to the transferrin receptor.
C11. The complex of any one of embodiments C1 to C10, wherein the antibody is cross-reactive with extracellular epitopes of two or more of a human, non-human primate and rodent transferrin receptor.
C12. The complex of any one of embodiments C1 to C11, wherein the complex is configured to promote transferrin receptor mediated internalization of the molecular payload into a muscle cell.
C13. The complex of any one of embodiments C1 to C12, wherein the molecular payload is an oligonucleotide comprising an antisense strand comprising a region of complementarity to a ACVR1 target sequence, optionally wherein the ACVR1 target sequence is an ACVR1 mRNA sequence as set forth in SEQ ID NO: 429 or SEQ ID NO: 430, or an ACVR1 target sequence as set forth in any one of SEQ ID NOs: 431-478.
C14. The complex of embodiment C13, wherein the antisense strand is 18-25 nucleotides in length and/or the region of complementarity is at least 16 nucleosides in length.
C15. The complex of embodiment C13 or embodiment C14, wherein the antisense strand comprises at least 16 consecutive nucleotides of a nucleotide sequence set forth in any one of SEQ ID NOs: 479-502, wherein each of the Us are optionally and independently Ts, optionally wherein the antisense strand comprises the nucleotide sequence of any one of SEQ ID NOs: 479-502, wherein each of the Us are optionally and independently Ts.
C16. The complex of any one of embodiments C13-C15, wherein the oligonucleotide further comprises a sense strand that hybridizes to the antisense strand to form a double stranded siRNA.
C17. The complex of any one of embodiments C13 to C16, wherein the oligonucleotide comprises one or more modified nucleosides, optionally wherein each nucleoside in the oligonucleotide is a modified nucleoside.
C18. The complex of embodiment 17, wherein the one or more modified nucleosides are 2′ modified nucleotides, optionally wherein the one or more 2′ modified nucleosides are selected from: 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethyl-bridged nucleic acid (cEt), optionally wherein the 2′ modified nucleotide is 2′-O-methyl or 2′-fluoro (2′-F).
C19. The complex of any one of embodiments C13 to C18, wherein the oligonucleotide comprises one or more phosphorothioate internucleoside linkages.
C20. The complex of embodiment C19, wherein the one or more phosphorothioate internucleoside linkage are present on the antisense strand of the RNAi oligonucleotide, further optionally wherein the two internucleoside linkages at the 3′ end of the sense strands are phosphorothioate internucleoside linkages.
C21. The complex of any one of embodiments C13 to C20, wherein the oligonucleotide is an siRNA listed in Table 19.
C22. The complex of any one of embodiments C1 to C21, wherein the antibody is covalently linked to the molecular payload via
(i) a cleavable linker, optionally wherein the cleavable linker comprises a valine-citrulline sequence; or
(ii) a non-cleavable linker, optionally wherein the non-cleavable linker is an alkane linker.
C23. A method of reducing ACVR1 expression in a muscle cell, the method comprising contacting the muscle cell with an effective amount of the complex of any one of embodiments C1 to C21 for promoting internalization of the molecular payload to the muscle cell.
C24. A method of treating a subject having a disease associated with elevated level of ACVR1, the method comprising administering to the subject an effective amount of the complex of any one of embodiments C1 to C21, optionally wherein the disease is muscle atrophy, further optionally wherein the muscle atrophy is sarcopenia or cachexia.
C25. The method of embodiment C23 or embodiment C24, wherein the complex reduces ACVR1 RNA level.
C26. The method of any one of embodiments C23 to C25, wherein the complex reduces ACVR1 protein level.
D1. A complex comprising an anti-transferrin receptor 1 antibody covalently linked to a molecular payload that modulates the expression or activity of atrogin-1 (FBXO32) or tripartite motif containing 63 (TRIM63), wherein the antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a heavy chain variable region (VH) comprising an amino acid sequence at least 85% identical to SEQ ID NO: 71; and/or a light chain variable region (VL) comprising an amino acid sequence at least 85% identical to SEQ ID NO: 70;
(iii) a heavy chain variable region (VH) comprising an amino acid sequence at least 85% identical to SEQ ID NO: 72; and/or a light chain variable region (VL) comprising an amino acid sequence at least 85% identical to SEQ ID NO: 70;
(iv) a heavy chain variable region (VH) comprising an amino acid sequence at least 85% identical to SEQ ID NO: 73; and/or a light chain variable region (VL) comprising an amino acid sequence at least 85% identical to SEQ ID NO: 74;
(v) a heavy chain variable region (VH) comprising an amino acid sequence at least 85% identical to SEQ ID NO: 73; and/or a light chain variable region (VL) comprising an amino acid sequence at least 85% identical to SEQ ID NO: 75;
(vi) a heavy chain variable region (VH) comprising an amino acid sequence at least 85% identical to SEQ ID NO: 76; and/or a light chain variable region (VL) comprising an amino acid sequence at least 85% identical to SEQ ID NO: 74;
(vii) a heavy chain variable region (VH) comprising an amino acid sequence at least 85% identical to SEQ ID NO: 69; and/or a light chain variable region (VL) comprising an amino acid sequence at least 85% identical to SEQ ID NO: 70;
(viii) a heavy chain variable region (VH) comprising an amino acid sequence at least 85% identical to SEQ ID NO: 77; and/or a light chain variable region (VL) comprising an amino acid sequence at least 85% identical to SEQ ID NO: 78;
(ix) a heavy chain variable region (VH) comprising an amino acid sequence at least 85% identical to SEQ ID NO: 79; and/or a light chain variable region (VL) comprising an amino acid sequence at least 85% identical to SEQ ID NO: 80; or
(x) a heavy chain variable region (VH) comprising an amino acid sequence at least 85% identical to SEQ ID NO: 77; and/or a light chain variable region (VL) comprising an amino acid sequence at least 85% identical to SEQ ID NO: 80.
D2. The complex of embodiment D1, wherein the antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 7land a VL comprising the amino acid sequence of SEQ ID NO: 70;
(iii) a VH comprising the amino acid sequence of SEQ ID NO: 72 and a VL comprising the amino acid sequence of SEQ ID NO: 70;
(iv) a VH comprising the amino acid sequence of SEQ ID NO: 73 and a VL comprising the amino acid sequence of SEQ ID NO: 74;
(v) a VH comprising the amino acid sequence of SEQ ID NO: 73 and a VL comprising the amino acid sequence of SEQ ID NO: 75;
(vi) a VH comprising the amino acid sequence of SEQ ID NO: 76 and a VL comprising the amino acid sequence of SEQ ID NO: 74;
(vii) a VH comprising the amino acid sequence of SEQ ID NO: 69 and a VL comprising the amino acid sequence of SEQ ID NO: 70;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
D3. The complex of embodiment D1 or embodiment D2, wherein the antibody is selected from the group consisting of a full-length IgG, a Fab fragment, a Fab′ fragment, a F(ab′)2 fragment, a scFv, and a Fv.
D4. The complex of embodiment D3, wherein the antibody is a full-length IgG, optionally wherein the full-length IgG comprises a heavy chain constant region of the isotype IgG1, IgG2, IgG3, or IgG4.
D5. The complex of embodiment D4, wherein the antibody comprises:
(i) a heavy chain comprising an amino acid sequence at least 85% identical to SEQ ID NO: 91; and/or a light chain comprising an amino acid sequence at least 85% identical to SEQ ID NO: 90;
(ii) a heavy chain comprising an amino acid sequence at least 85% identical to SEQ ID NO: 86; and/or a light chain comprising an amino acid sequence at least 85% identical to SEQ ID NO: 85;
(iii) a heavy chain comprising an amino acid sequence at least 85% identical to SEQ ID NO: 87; and/or a light chain comprising an amino acid sequence at least 85% identical to SEQ ID NO: 85;
(iv) a heavy chain comprising an amino acid sequence at least 85% identical to SEQ ID NO: 88; and/or a light chain comprising an amino acid sequence at least 85% identical to SEQ ID NO: 89;
(v) a heavy chain comprising an amino acid sequence at least 85% identical to SEQ ID NO: 88; and/or a light chain comprising an amino acid sequence at least 85% identical to SEQ ID NO: 90;
(vi) a heavy chain comprising an amino acid sequence at least 85% identical to SEQ ID NO: 91; and/or a light chain comprising an amino acid sequence at least 85% identical to SEQ ID NO: 89;
(vii) a heavy chain comprising an amino acid sequence at least 85% identical to SEQ ID NO: 84; and/or a light chain comprising an amino acid sequence at least 85% identical to SEQ ID NO: 85;
(viii) a heavy chain comprising an amino acid sequence at least 85% identical to SEQ ID NO: 92; and/or a light chain comprising an amino acid sequence at least 85% identical to SEQ ID NO: 93;
(ix) a heavy chain comprising an amino acid sequence at least 85% identical to SEQ ID NO: 94; and/or a light chain comprising an amino acid sequence at least 85% identical to SEQ ID NO: 95; or
(x) a heavy chain comprising an amino acid sequence at least 85% identical to SEQ ID NO: 92; and/or a light chain comprising an amino acid sequence at least 85% identical to SEQ ID NO: 95.
D6. The complex of embodiment D3, wherein the antibody is a Fab fragment.
D7. The complex of embodiment D6, wherein the antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 98; and/or a light chain comprising an amino acid sequence at least 85% identical to SEQ ID NO: 85;
(iii) a heavy chain comprising an amino acid sequence at least 85% identical to SEQ ID NO: 99; and/or a light chain comprising an amino acid sequence at least 85% identical to SEQ ID NO: 85;
(iv) a heavy chain comprising an amino acid sequence at least 85% identical to SEQ ID NO: 100; and/or a light chain comprising an amino acid sequence at least 85% identical to SEQ ID NO: 89;
(v) a heavy chain comprising an amino acid sequence at least 85% identical to SEQ ID NO: 100; and/or a light chain comprising an amino acid sequence at least 85% identical to SEQ ID NO: 90;
(vi) a heavy chain comprising an amino acid sequence at least 85% identical to SEQ ID NO: 101; and/or a light chain comprising an amino acid sequence at least 85% identical to SEQ ID NO: 89;
(vii) a heavy chain comprising an amino acid sequence at least 85% identical to SEQ ID NO: 97; and/or a light chain comprising an amino acid sequence at least 85% identical to SEQ ID NO: 85;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
D8. The complex of embodiment D6 or embodiment D7, wherein the antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 98; and a light chain comprising the amino acid sequence of SEQ ID NO: 85;
(iii) a heavy chain comprising the amino acid sequence of SEQ ID NO: 99; and a light chain comprising the amino acid sequence of SEQ ID NO: 85;
(iv) a heavy chain comprising the amino acid sequence of SEQ ID NO: 100; and a light chain comprising the amino acid sequence of SEQ ID NO: 89;
(v) a heavy chain comprising the amino acid sequence of SEQ ID NO: 100; and a light chain comprising the amino acid sequence of SEQ ID NO: 90;
(vi) a heavy chain comprising the amino acid sequence of SEQ ID NO: 101; and a light chain comprising the amino acid sequence of SEQ ID NO: 89;
(vii) a heavy chain comprising the amino acid sequence of SEQ ID NO: 97; and a light chain comprising the amino acid sequence of SEQ ID NO: 85;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
D9. The complex of any one of embodiments D1 to D8, wherein the equilibrium dissociation constant (K_{D}) of binding of the antibody to the transferrin receptor is in a range from 10⁻¹¹ M to 10⁻⁶ M.
D10. The complex of any one of embodiments D1 to D9, wherein the antibody does not specifically bind to the transferrin binding site of the transferrin receptor and/or wherein the antibody does not inhibit binding of transferrin to the transferrin receptor.
D11. The complex of any one of embodiments D1 to D10, wherein the antibody is cross-reactive with extracellular epitopes of two or more of a human, non-human primate and rodent transferrin receptor.
D12. The complex of any one of embodiments D1 to D11, wherein the complex is configured to promote transferrin receptor mediated internalization of the molecular payload into a muscle cell.
D13. The complex of any one of embodiments D1 to D12, wherein the molecular payload is an oligonucleotide comprising an antisense strand comprising a region of complementarity to a FBXO32 target sequence, optionally wherein the FBXO32 target sequence is an FBXO32 mRNA sequence as set forth in SEQ ID NO: 505 or SEQ ID NO: 506, or a FBXO32 target sequence as set forth in any one of SEQ ID NOs: 507-554, further optionally wherein the antisense strand is 18-25 nucleotides in length and/or the region of complementarity is at least 16 nucleosides in length.
D14. The complex of embodiment D13, wherein the antisense strand comprises at least 16 consecutive nucleotides of a nucleotide sequence set forth in any one of SEQ ID NOs: 555-578, wherein each of the Us are optionally and independently Ts, optionally wherein the antisense strand comprises the nucleotide sequence of any one of SEQ ID NOs: 555-578, wherein each of the Us are optionally and independently Ts.
D15. The complex of any one of embodiments D1 to D12, wherein the molecular payload is an oligonucleotide comprising an antisense strand comprising a region of complementarity to TRIM63 target sequence, optionally wherein the TRIM63 target sequence is a TRIM63 mRNA sequence as set forth in SEQ ID NO: 579 or SEQ ID NO: 580, or a TRIM63 target sequence as set forth in any one of SEQ ID NOs: 581-628, further optionally wherein the antisense strand is 18-25 nucleotides in length and/or the region of complementarity is at least 16 nucleosides in length.
D16. The complex of embodiment D15, wherein the antisense strand comprises at least 16 consecutive nucleotides of a nucleotide sequence set forth in any one of SEQ ID NOs: 629-652, wherein each of the Us are optionally and independently Ts, optionally wherein the antisense strand comprises the nucleotide sequence of any one of SEQ ID NOs: 629-652, wherein each of the Us are optionally and independently Ts.
D17. The complex of any one of embodiments D13 to D16, wherein the oligonucleotide further comprises a sense strand that hybridizes to the antisense strand to form a double stranded siRNA.
D18. The complex of any one of embodiments D13 to D17, wherein the oligonucleotide comprises one or more modified nucleosides, optionally wherein each nucleoside in the oligonucleotide is a modified nucleoside.
D19. The complex of embodiment D18, wherein the one or more modified nucleosides are 2′ modified nucleotides, optionally wherein the one or more 2′ modified nucleosides are selected from: 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethyl-bridged nucleic acid (cEt), optionally wherein the 2′ modified nucleotide is 2′-O-methyl or 2′-fluoro (2′-F).
D20. The complex of any one of embodiments D13 to D19, wherein the oligonucleotide comprises one or more phosphorothioate internucleoside linkages, optionally wherein the one or more phosphorothioate internucleoside linkage are present on the antisense strand of the RNAi oligonucleotide, further optionally wherein the two internucleoside linkages at the 3′ end of the sense strands are phosphorothioate internucleoside linkages.
D21. The complex of any one of embodiments D13, D14, and D17 to D20, wherein the oligonucleotide is an siRNA listed in Table 22.
D22. The complex of any one of embodiments D15 to D20, wherein the oligonucleotide is an siRNA listed in Table 25.
D23. The complex of any one of embodiments D1 to D22, wherein the antibody is covalently linked to the molecular payload via
(i) a cleavable linker, optionally wherein the cleavable linker comprises a valine-citrulline dipeptide sequence; or
(ii) a non-cleavable linker, optionally wherein the non-cleavable linker is an alkane linker.
D24. A method of reducing FBXO32 or TRIM63 expression in a muscle cell, the method comprising contacting the muscle cell with an effective amount of the complex of any one of embodiments 1-23 for promoting internalization of the molecular payload to the muscle cell.
D25. A method of treating muscle atrophy the method comprising administering to a subject in need thereof an effective amount of the complex of any one of embodiments 1-23, wherein the subject has elevated expression or activity of FBXO32 or TRIM63.
D26. The method of embodiment D24 or embodiment D25, wherein the complex reduces RNA level of FBXO32 or TRIM63.
D27. The method of any one of embodiments D24 to D26, wherein the complex reduces protein level of FBXO32 or TRIM63.
E1. A complex comprising an anti-transferrin receptor 1 antibody covalently linked to a molecular payload that modulates the expression or activity of MEF2D, KLF15, MED1, MED13, or PPP1R3A, wherein the antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a heavy chain variable region (VH) comprising an amino acid sequence at least 85% identical to SEQ ID NO: 71; and/or a light chain variable region (VL) comprising an amino acid sequence at least 85% identical to SEQ ID NO: 70;
(iii) a heavy chain variable region (VH) comprising an amino acid sequence at least 85% identical to SEQ ID NO: 72; and/or a light chain variable region (VL) comprising an amino acid sequence at least 85% identical to SEQ ID NO: 70;
(iv) a heavy chain variable region (VH) comprising an amino acid sequence at least 85% identical to SEQ ID NO: 73; and/or a light chain variable region (VL) comprising an amino acid sequence at least 85% identical to SEQ ID NO: 74;
(v) a heavy chain variable region (VH) comprising an amino acid sequence at least 85% identical to SEQ ID NO: 73; and/or a light chain variable region (VL) comprising an amino acid sequence at least 85% identical to SEQ ID NO: 75;
(vi) a heavy chain variable region (VH) comprising an amino acid sequence at least 85% identical to SEQ ID NO: 76; and/or a light chain variable region (VL) comprising an amino acid sequence at least 85% identical to SEQ ID NO: 74;
(vii) a heavy chain variable region (VH) comprising an amino acid sequence at least 85% identical to SEQ ID NO: 69; and/or a light chain variable region (VL) comprising an amino acid sequence at least 85% identical to SEQ ID NO: 70;
(viii) a heavy chain variable region (VH) comprising an amino acid sequence at least 85% identical to SEQ ID NO: 77; and/or a light chain variable region (VL) comprising an amino acid sequence at least 85% identical to SEQ ID NO: 78;
(ix) a heavy chain variable region (VH) comprising an amino acid sequence at least 85% identical to SEQ ID NO: 79; and/or a light chain variable region (VL) comprising an amino acid sequence at least 85% identical to SEQ ID NO: 80; or
(x) a heavy chain variable region (VH) comprising an amino acid sequence at least 85% identical to SEQ ID NO: 77; and/or a light chain variable region (VL) comprising an amino acid sequence at least 85% identical to SEQ ID NO: 80.
E2. The complex of embodiment E1, wherein the antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 7land a VL comprising the amino acid sequence of SEQ ID NO: 70;
(iii) a VH comprising the amino acid sequence of SEQ ID NO: 72 and a VL comprising the amino acid sequence of SEQ ID NO: 70;
(iv) a VH comprising the amino acid sequence of SEQ ID NO: 73 and a VL comprising the amino acid sequence of SEQ ID NO: 74;
(v) a VH comprising the amino acid sequence of SEQ ID NO: 73 and a VL comprising the amino acid sequence of SEQ ID NO: 75;
(vi) a VH comprising the amino acid sequence of SEQ ID NO: 76 and a VL comprising the amino acid sequence of SEQ ID NO: 74;
(vii) a VH comprising the amino acid sequence of SEQ ID NO: 69 and a VL comprising the amino acid sequence of SEQ ID NO: 70;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
E3. The complex of embodiment E1 or embodiment E2, wherein the antibody is selected from the group consisting of a full-length IgG, a Fab fragment, a Fab′ fragment, a F(ab′)2 fragment, a scFv, and a Fv.
E4. The complex of embodiment E3, wherein the antibody is a full-length IgG, optionally wherein the full-length IgG comprises a heavy chain constant region of the isotype IgG1, IgG2, IgG3, or IgG4.
E5. The complex of embodiment E4, wherein the antibody comprises:
(i) a heavy chain comprising an amino acid sequence at least 85% identical to SEQ ID NO: 91; and/or a light chain comprising an amino acid sequence at least 85% identical to SEQ ID NO: 90;
(ii) a heavy chain comprising an amino acid sequence at least 85% identical to SEQ ID NO: 86; and/or a light chain comprising an amino acid sequence at least 85% identical to SEQ ID NO: 85;
(iii) a heavy chain comprising an amino acid sequence at least 85% identical to SEQ ID NO: 87; and/or a light chain comprising an amino acid sequence at least 85% identical to SEQ ID NO: 85;
(iv) a heavy chain comprising an amino acid sequence at least 85% identical to SEQ ID NO: 88; and/or a light chain comprising an amino acid sequence at least 85% identical to SEQ ID NO: 89;
(v) a heavy chain comprising an amino acid sequence at least 85% identical to SEQ ID NO: 88; and/or a light chain comprising an amino acid sequence at least 85% identical to SEQ ID NO: 90;
(vi) a heavy chain comprising an amino acid sequence at least 85% identical to SEQ ID NO: 91; and/or a light chain comprising an amino acid sequence at least 85% identical to SEQ ID NO: 89;
(vii) a heavy chain comprising an amino acid sequence at least 85% identical to SEQ ID NO: 84; and/or a light chain comprising an amino acid sequence at least 85% identical to SEQ ID NO: 85;
(viii) a heavy chain comprising an amino acid sequence at least 85% identical to SEQ ID NO: 92; and/or a light chain comprising an amino acid sequence at least 85% identical to SEQ ID NO: 93;
(ix) a heavy chain comprising an amino acid sequence at least 85% identical to SEQ ID NO: 94; and/or a light chain comprising an amino acid sequence at least 85% identical to SEQ ID NO: 95; or
(x) a heavy chain comprising an amino acid sequence at least 85% identical to SEQ ID NO: 92; and/or a light chain comprising an amino acid sequence at least 85% identical to SEQ ID NO: 95.
E6. The complex of embodiment E3, wherein the antibody is a Fab fragment.
E7. The complex of embodiment E6, wherein the antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 98; and/or a light chain comprising an amino acid sequence at least 85% identical to SEQ ID NO: 85;
(iii) a heavy chain comprising an amino acid sequence at least 85% identical to SEQ ID NO: 99; and/or a light chain comprising an amino acid sequence at least 85% identical to SEQ ID NO: 85;
(iv) a heavy chain comprising an amino acid sequence at least 85% identical to SEQ ID NO: 100; and/or a light chain comprising an amino acid sequence at least 85% identical to SEQ ID NO: 89;
(v) a heavy chain comprising an amino acid sequence at least 85% identical to SEQ ID NO: 100; and/or a light chain comprising an amino acid sequence at least 85% identical to SEQ ID NO: 90;
(vi) a heavy chain comprising an amino acid sequence at least 85% identical to SEQ ID NO: 101; and/or a light chain comprising an amino acid sequence at least 85% identical to SEQ ID NO: 89;
(vii) a heavy chain comprising an amino acid sequence at least 85% identical to SEQ ID NO: 97; and/or a light chain comprising an amino acid sequence at least 85% identical to SEQ ID NO: 85;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
E8. The complex of embodiment E6 or embodiment E7, wherein the antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 98; and a light chain comprising the amino acid sequence of SEQ ID NO: 85;
(iii) a heavy chain comprising the amino acid sequence of SEQ ID NO: 99; and a light chain comprising the amino acid sequence of SEQ ID NO: 85;
(iv) a heavy chain comprising the amino acid sequence of SEQ ID NO: 100; and a light chain comprising the amino acid sequence of SEQ ID NO: 89;
(v) a heavy chain comprising the amino acid sequence of SEQ ID NO: 100; and a light chain comprising the amino acid sequence of SEQ ID NO: 90;
(vi) a heavy chain comprising the amino acid sequence of SEQ ID NO: 101; and a light chain comprising the amino acid sequence of SEQ ID NO: 89;
(vii) a heavy chain comprising the amino acid sequence of SEQ ID NO: 97; and a light chain comprising the amino acid sequence of SEQ ID NO: 85;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
E9. The complex of any one of embodiments E1 to E8, wherein the equilibrium dissociation constant (K_{D}) of binding of the antibody to the transferrin receptor is in a range from 10⁻¹¹ M to 10⁻⁶ M.
E10. The complex of any one of embodiments E1 to E9, wherein the antibody does not specifically bind to the transferrin binding site of the transferrin receptor and/or wherein the antibody does not inhibit binding of transferrin to the transferrin receptor.
E11. The complex of any one of embodiments E1 to E10, wherein the antibody is cross-reactive with extracellular epitopes of two or more of a human, non-human primate and rodent transferrin receptor.
E12. The complex of any one of embodiments E1 to E11, wherein the complex is configured to promote transferrin receptor mediated internalization of the molecular payload into a muscle cell.
E13. The complex of any one of embodiments E1 to E12, wherein the molecular payload is an oligonucleotide comprising an antisense strand comprising a region of complementarity to a MEF2D target sequence, optionally wherein the MEF2D target sequence is an MEF2D mRNA sequence as set forth in SEQ ID NO: 664 or SEQ ID NO: 665, or a MEF2D target sequence as set forth in any one of SEQ ID NOs: 668-715, further optionally wherein the antisense strand is 18-25 nucleotides in length and/or the region of complementarity is at least 16 nucleosides in length.
E14. The complex of embodiment E13, wherein the antisense strand comprises at least 16 consecutive nucleotides of a nucleotide sequence set forth in any one of SEQ ID NOs: 716-223,
wherein each of the Us are optionally and independently Ts, optionally wherein the antisense strand comprises the nucleotide sequence of any one of SEQ ID NOs: 716-223, wherein each of the Us are optionally and independently Ts.
E15. The complex of any one of embodiments E1 to E12, wherein the molecular payload is an oligonucleotide comprising an antisense strand comprising a region of complementarity to KLF15 mRNA, optionally wherein the KLF15 target sequence is a KLF15 mRNA sequence as set forth in SEQ ID NO: 740 or SEQ ID NO: 741, or a KLF15 target sequence as set forth in any one of SEQ ID NOs: 742-789, further optionally wherein the antisense strand is 18-25 nucleotides in length and/or the region of complementarity is at least 16 nucleosides in length.
E16. The complex of embodiment E15, wherein the antisense strand comprises at least 16 consecutive nucleotides of a nucleotide sequence set forth in any one of SEQ ID NOs: 790-813,
wherein each of the Us are optionally and independently Ts, optionally wherein the antisense strand comprises the nucleotide sequence of any one of SEQ ID NOs: 790-813, wherein each of the Us are optionally and independently Ts.
E17. The complex of any one of embodiments E1 to E12, wherein the molecular payload is an oligonucleotide comprising an antisense strand comprising a region of complementarity to MED1 mRNA, optionally wherein the MED1 target sequence is a MED1 mRNA sequence as set forth in SEQ ID NO: 814 or SEQ ID NO: 815, or a MED1 target sequence as set forth in any one of SEQ ID NOs: 816-863, further optionally wherein the antisense strand is 18-25 nucleotides in length and/or the region of complementarity is at least 16 nucleosides in length.
E18. The complex of embodiment E17, wherein the antisense strand comprises at least 16 consecutive nucleotides of a nucleotide sequence set forth in any one of SEQ ID NOs: 864-887,
wherein each of the Us are optionally and independently Ts, optionally wherein the antisense strand comprises the nucleotide sequence of any one of SEQ ID NOs: 864-887, wherein each of the Us are optionally and independently Ts.
E19. The complex of any one of embodiments E1 to E12, wherein the molecular payload is an oligonucleotide comprising an antisense strand comprising a region of complementarity to MED13 mRNA, optionally wherein the MED13 target sequence is a MED13 mRNA sequence as set forth in SEQ ID NO: 888 or SEQ ID NO: 889, or a MED13 target sequence as set forth in any one of SEQ ID NOs: 890-937, further optionally wherein the antisense strand is 18-25 nucleotides in length and/or the region of complementarity is at least 16 nucleosides in length.
E20. The complex of embodiment E19, wherein the antisense strand comprises at least 16 consecutive nucleotides of a nucleotide sequence set forth in any one of SEQ ID NOs: 938-961,
wherein each of the Us are optionally and independently Ts, optionally wherein the antisense strand comprises the nucleotide sequence of any one of SEQ ID NOs: 938-961, wherein each of the Us are optionally and independently Ts.
E21. The complex of any one of embodiments E1 to E12, wherein the molecular payload is an oligonucleotide comprising an antisense strand comprising a region of complementarity to PPP1R3A mRNA, optionally wherein the PPP1R3A target sequence is a PPP1R3A mRNA sequence as set forth in SEQ ID NO: 962 or SEQ ID NO: 963, or a PPP1R3A target sequence as set forth in any one of SEQ ID NOs: 964-1011, further optionally wherein the antisense strand is 18-25 nucleotides in length and/or the region of complementarity is at least 16 nucleosides in length.
E22. The complex of embodiment E21, wherein the antisense strand comprises at least 16 consecutive nucleotides of a nucleotide sequence set forth in any one of SEQ ID NOs: 1012-1035,
wherein each of the Us are optionally and independently Ts, optionally wherein the antisense strand comprises the nucleotide sequence of any one of SEQ ID NOs: 1012-1035, wherein each of the Us are optionally and independently Ts.
E23. The complex of any one of embodiments E13 to E22, wherein the oligonucleotide further comprises a sense strand that hybridizes to the antisense strand to form a double stranded siRNA.
E24. The complex of any one of embodiments E13 to E23, wherein the oligonucleotide comprises one or more modified nucleosides, optionally wherein each nucleoside in the oligonucleotide is a modified nucleoside.
E25. The complex of embodiment E24, wherein the one or more modified nucleosides are 2′ modified nucleotides, optionally wherein the one or more 2′ modified nucleosides are selected from: 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), ethylene-bridged nucleic acid (ENA), and (S)-constrained ethyl-bridged nucleic acid (cEt), optionally wherein the 2′ modified nucleotide is 2′-O-methyl or 2′-fluoro (2′-F).
E26. The complex of any one of embodiments E13 to E25, wherein the oligonucleotide comprises one or more phosphorothioate internucleoside linkages, optionally wherein the one or more phosphorothioate internucleoside linkage are present on the antisense strand of the RNAi oligonucleotide, further optionally wherein the two internucleoside linkages at the 3′ end of the sense strands are phosphorothioate internucleoside linkages. E27. The complex of any one of embodiments E13, E14 and E23 to E26, wherein the oligonucleotide is an siRNA listed in Table 28.
E28. The complex of any one of embodiments E15, E16, and E23 to E26, wherein the oligonucleotide is an siRNA listed in Table 31.
E29. The complex of any one of embodiments E17, E18, and E23 to E26, wherein the oligonucleotide is an siRNA listed in Table 34.
E30. The complex of any one of embodiments E19, E20, and E23 to E26, wherein the oligonucleotide is an siRNA listed in Table 37.
E31. The complex of any one of embodiments E21 to E26, wherein the oligonucleotide is an siRNA listed in Table 40.
E32. The complex of any one of embodiments E1 to E31, wherein the antibody is covalently linked to the molecular payload via
(i) a cleavable linker, optionally wherein the cleavable linker comprises a valine-citrulline sequence; or
(ii) a non-cleavable linker, optionally wherein the non-cleavable linker is an alkane linker.
E33. A method of reducing MEF2D, KLF15, MED1, MED13, or PPP1R3A expression in a muscle cell, the method comprising contacting the muscle cell with an effective amount of the complex of any one of embodiments E1 to E32 for promoting internalization of the molecular payload to the muscle cell.
E34. A method of treating a heart disease, the method comprising administering to a subject in need thereof an effective amount of the complex of any one of embodiments E1 to E32, wherein the subject has elevated expression or activity of MEF2D, KLF15, MED1, MED13, or PPP1R3A.
E35. The method of embodiment E33 or embodiment E34, wherein the complex reduces RNA level of MEF2D, KLF15, MED1, MED13, or PPP1R3A.
E36. The method of any one of embodiments E33 to E35, wherein the complex reduces protein level of MEF2D, KLF15, MED1, MED13, or PPP1R3A

### Quivalents and Terminology

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor 1 antibody covalently linked to a molecular payload that modulates the expression or activity of myostatin (MSTN), inhibin beta A (INHBA), activin receptor type-1B (ACVR1B), myosin light chain kinase (MLCK1), activin A receptor type-1 (ACVR1), atrogin-1 (FBXO32), tripartite motif containing 63 (TRIM63), myocyte-specific enhancer factor 2D (MEF2D), Krüppel-like factor 15 (KLF15), Mediator complex subunit 1 (MED1), Mediator complex subunit 13 (MED13), and/or protein phosphatase 1 regulatory subunit 3A (PPP1R3A), wherein the antibody comprises:
a heavy chain variable region (VH) comprising an amino acid sequence at least 95% identical to SEQ ID NO: 72; and/or a light chain variable region (VL) comprising an amino acid sequence at least 95% identical to SEQ ID NO: 70.

2. The complex of claim 1, wherein the antibody comprises:
a VH comprising the amino acid sequence of SEQ ID NO: 72 and a VL comprising the amino acid sequence of SEQ ID NO: 70.

3. The complex of claim 1, wherein the antibody is selected from the group consisting of a full-length IgG, a Fab fragment, a Fab′ fragment, a F(ab′)2 fragment, a scFv, and a Fv.

4. The complex of claim 3, wherein the antibody is a full-length IgG, and wherein the full-length IgG comprises a heavy chain constant region of the isotype IgG1, IgG2, IgG3, or IgG4.

5. The complex of claim 4, wherein the antibody comprises:
a heavy chain comprising an amino acid sequence at least 85% identical to SEQ ID NO: 87; and/or a light chain comprising an amino acid sequence at least 85% identical to SEQ ID NO: 85.

6. The complex of claim 3 wherein the antibody is a Fab.

7. The complex of claim 6, wherein the antibody comprises:
a heavy chain comprising an amino acid sequence at least 85% identical to SEQ ID NO: 99; and/or a light chain comprising an amino acid sequence at least 85% identical to SEQ ID NO: 85.

8. The complex of claim 6, wherein the antibody comprises:
a heavy chain comprising the amino acid sequence of SEQ ID NO: 99; and a light chain comprising the amino acid sequence of SEQ ID NO: 85.

9. The complex of claim 1, wherein the equilibrium dissociation constant (K_{D}) of binding of the antibody to the transferrin receptor is in a range from 10⁻¹¹ M to 10⁻⁶ M.

10.-35. (canceled)

36. The complex of claim 1, wherein the molecular payload comprises an oligonucleotide comprising an antisense strand comprising a region of complementarity to a target sequence, and further comprises a sense strand that hybridizes to the antisense strand to form a double stranded siRNA.

37. The complex of claim 36, wherein the oligonucleotide comprises one or more modified nucleosides, and/or wherein the oligonucleotide comprises one or more phosphorothioate internucleoside linkages.

38.-39. (canceled)

40. The complex of claim 36, wherein the oligonucleotide is an siRNA listed in Table 10, Table 13, Table 16, Table 19, Table 22, Table 25, Table 28, Table 31, Table 34, Table 37, or Table 40.

41. The complex of claim 1, wherein the antibody is covalently linked to the molecular payload via
(i) a cleavable linker, wherein the cleavable linker comprises a valine-citrulline sequence; or
(ii) a non-cleavable linker, wherein the non-cleavable linker is an alkane linker.

42. A method of reducing MSTN, INHBA, ACVR1B, MLCK1, ACVR1, FBXO32, TRIM63, MEF2D, KLF15, MED1, MED13, and/or PPP1R3A expression in a muscle cell, the method comprising contacting the muscle cell with an effective amount of the complex of claim 1 for promoting internalization of the molecular payload to the muscle cell.

43. A method of treating muscle atrophy the method comprising administering to a subject in need thereof an effective amount of the complex of claim 1, wherein the subject has elevated expression or activity of MSTN, INHBA, ACVR1B, FBXO32 and/or TRIM63, and the complex comprises a molecular payload that modulates the expression or activity of MSTN, INHBA, ACVR1B, FBXO32 and/or TRIM63, wherein the subject is human, and wherein the administration is intravenous.

44. A method of treating irritable bowel syndrome (IBS) or irritable bowel disease (IBD) the method comprising administering to a subject in need thereof an effective amount of the complex of claim 1, wherein the subject has elevated levels of MLCK1 protein and the complex comprises a molecular payload that modulates the expression or activity of MLCK1, wherein the subject is human, and wherein the administration is intravenous.

45. A method of treating a subject having a disease associated with elevated level of ACVR1, the method comprising administering to the subject an effective amount of the complex of claim 1, wherein the complex comprises a molecular payload that modulates the expression or activity of ACVR1, wherein the disease is muscle atrophy, further wherein the muscle atrophy is sarcopenia or cachexia, further wherein the subject is human, and wherein the administration is intravenous.

46. (canceled)

47. A method of treating a heart disease, the method comprising administering to a subject in need thereof an effective amount of the complex of claim 1, wherein the subject has elevated expression or activity of MEF2D, KLF15, MED1, MED13, and/or PPP1R3A, and the complex comprises a molecular payload that modulates the expression or activity of MEF2D, KLF15, MED1, MED13, and/or PPP1R3A, wherein the subject is human, and wherein the administration is intravenous.

48. The method of claim 42, wherein the complex reduces RNA level and/or protein level of MSTN, INHBA, ACVR1B, MLCK1, ACVR1, FBXO32, TRIM63, MEF2D, KLF15, MED1, MED13, and/or PPP1R3A.

49. (canceled)

50. An oligonucleotide comprising an siRNA listed in Tables 9, 10, 12, 13, 15, 16, 18, 19, 21, 22, 24, 25, 27, 28, 30, 21, 33, 34, 36, 37, 39, and 40.
